(19) United States
(12) Patent Application Publication
(10) Pub. No.: US 2025/0179085 A1
(43) Pub. Date: Jun. 5, 2025

(54) HETEROCYCLIC COMPOUND, AND ORGANIC LIGHT-EMITTING DEVICE AND ORGANIC MATERIAL LAYER FORMATION COMPOSITION WHICH COMPRISE SAME

(71) Applicant: LT MATERIALS CO., LTD., Yongin-si, Gyeonggi-do (KR)

(72) Inventors: Seong-Jong PARK, Yongin-si (KR); Young-Seok NO, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/841,759

(22) PCT Filed: Jan. 18, 2023
(86) PCT No.: PCT/KR2023/000878

§ 371 Date: Aug. 27, 2024

(30) Foreign Application Priority Data
Feb. 28, 2022 (KR) 10-2022-0025928

(51) Int. Cl. C07D 491/052; C07D 495/00; H10K 50/12; H10K 85/60

(52) U.S. Cl. CPC C07D 491/052; C07D 495/00; H10K 50/12; H10K 85/622; H10K 85/654; H10K 85/6572; H10K 85/6574; H10K 85/6576

(57) ABSTRACT

The present specification relates to a heterocyclic compound of Chemical Formula 1, an organic light emitting device including the same and a composition for forming an organic material layer.

### TECHNICAL FIELD

The present specification relates to a heterocyclic compound, an organic light emitting device including the same and a composition for forming an organic material layer.

This application claims priority to and the benefit of Korean Patent Application No. 10-2022-0025928 filed in the Korean Intellectual Property Office on Feb. 28, 2022, the entire contents of which are incorporated herein by reference.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may play a role such as a hole injection, hole transport, electron blocking, hole blocking, electron transport or electron injection.

In order to improve the performance, service life, or efficiency of the organic light emitting device, there is a continuous need for developing a material for an organic thin film.
[Citation List] (Patent Document 1) U.S. Pat. No. 4,356,429

### DETAILED DESCRIPTION OF THE INVENTION

### Technical Problem

The present specification has been made in an effort to provide a heterocyclic compound, an organic light emitting device including the same and a composition for forming an organic material layer.

### Technical Solution

In an exemplary embodiment of present specification, provided is a heterocyclic compound of the following Chemical Formula 1.

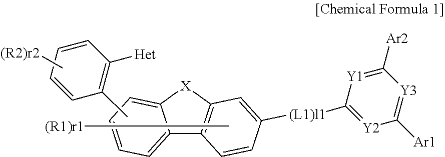

In Chemical Formula 1,
X is O or S,
L1 is a direct bond; a substituted or unsubstituted C6 to C60 arylene group; or a substituted or unsubstituted C2 to C60 heteroarylene group,
Y1 to Y3 are each independently N or CR, and at least one thereof is N,
Het is a substituted or unsubstituted C2 to C60 fused polycyclic heteroaryl group,
R, R1, Ar1, and Ar2 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R2 is hydrogen; or deuterium,
l1 is an integer from 0 to 3,
r1 is an integer from 0 to 6,
r2 is an integer from 0 to 4, and
when l1, r1, and r2 are each 2 or higher, substituents in the parenthesis are the same as or different from each other.

In another exemplary embodiment of the present specification, provided is an organic light emitting device including: a first electrode; a second electrode; and an organic material layer having one or more layers provided between the first electrode and the second electrode, in which one or more layers of the organic material layer include one or more of the heterocyclic compounds.

In still another exemplary embodiment of the present specification, provided is a composition for forming an organic material layer, including the heterocyclic compound.

### Advantageous Effects

When used in an organic light emitting device, the heterocyclic compound described in the present specification can lower the driving voltage of the device, improve the light emitting efficiency, and improve the service life characteristics of the device. Specifically, the heterocyclic compound of the present invention is characterized in that an azine group is substituted at a specific position of dibenzofuran or dibenzothiophene which has a core structure, a phenylene group is included as a linker between the core structure and a substituent Het, and the core structure and the substituent Het are bonded at the ortho position based on the phenylene group. In particular, the substituent Het is bonded to the core structure at the ortho position of the phenylene group, which increases the dihedral angle compared to when the substituent Het is bonded thereto at the meta or para position. As the dihedral angle increases, the extension of conjugation based on the phenylene group is suppressed, so that the substituent Het at the ortho position has a higher T₁ (triplet energy level) than that at the meta or para positions. The high T₁ (triplet energy level) of a phosphorescent host material is suitable for maximizing the light emitting characteristics of a phosphorescent dopant. When a phosphorescent host material having a low T₁ (triplet energy level) is used, a back energy transfer occurs in which the electrons of the dopant in the triplet state are transferred back to the host, degrading the light emitting characteristics of the dopant to degrade the device performance. In the present invention, as shown in Chemical Formula 1, by using a compound having a high T₁ (triplet energy level), in which the core structure and the substituent Het are bonded at the ortho position based on the phenylene group, there is an effect that the light emitting characteristics of the dopant are maximized by preventing back energy transfer, thereby improving the device performance.

In addition, when the azine group in the core structure is bonded to the No. 3 position of dibenzofuran or dibenzothiophene, the compound has a higher glass transition temperature than a compound having the azine group bonded to other positions. When the glass transition temperature is low, the material loses its original characteristics has completely different characteristics, and has a reduced service life. In the present invention, there is an effect that the service life is improved by substituting an azine group at No. 3 position of dibenzofuran or dibenzothiophene, which has a high glass transition temperature.

### BRIEF DESCRIPTION OF DRAWINGS

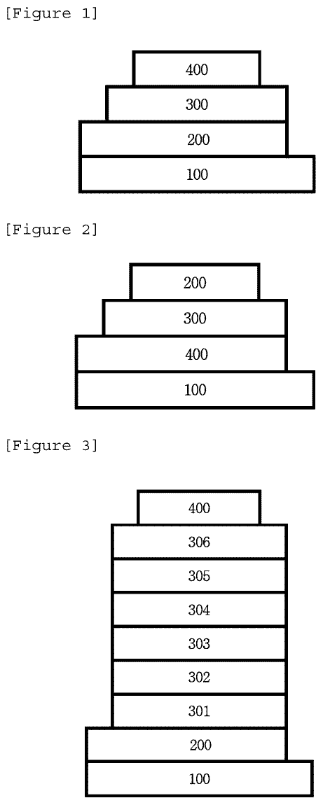

FIGS. 1 to 3 are views each exemplarily illustrating a stacking structure of an organic light emitting device according to an exemplary embodiment of the present specification.
100: Substrate
200: Positive electrode
300: Organic material layer
301: Hole injection layer
302: Hole transport layer
303: Light Emitting Layer
304: Hole blocking layer
305: Electron transport layer
306: Electron injection layer
400: Negative electrode

### BEST MODE

Hereinafter, the present specification will be described in more detail.

When one part “includes” one constituent element in the present specification, unless otherwise specifically described, this does not mean that another constituent element is excluded, but means that another constituent element may be further included.

In the present specification,

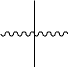

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent to which two or more substituents selected among the exemplified substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

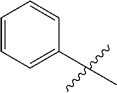 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

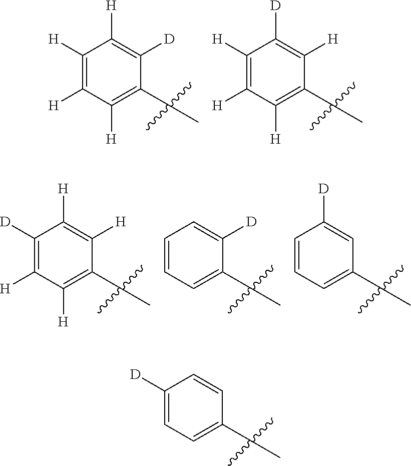

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O)₂(R105), and the above-described examples of the alkyl group may be applied to R105.

In the present specification, an arylsulfoxy group is represented by —S(═O)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

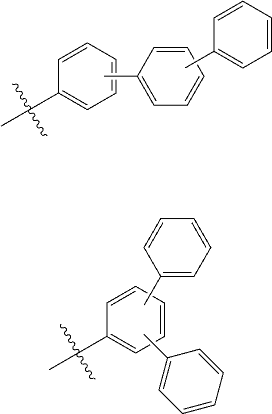

In the present specification, the fluorenyl group may be substituted, and adjacent substituents may be bonded to each other to form a ring.

When the fluorenyl group is substituted, the substituent may be the following structures, but is not limited thereto.

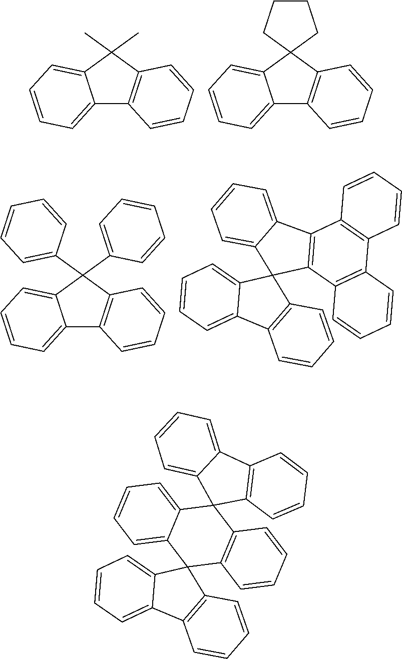

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi (dibenzosilole), a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, a benzocarbazole group may be any one of the following structures.

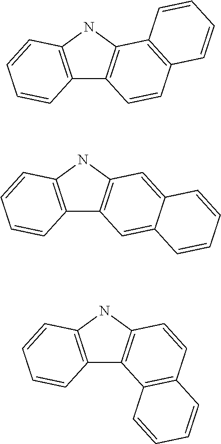 [SMILES: c1ccc2c(c1)ccc1c3ccccc3[nH]c21 ; c1ccc2cc3c(cc2c1)[nH]c1ccccc13 ; c1ccc2c(c1)ccc1[nH]c3ccccc3c12]

In the present specification, a dibenzocarbazole group may be any one of the following structures.

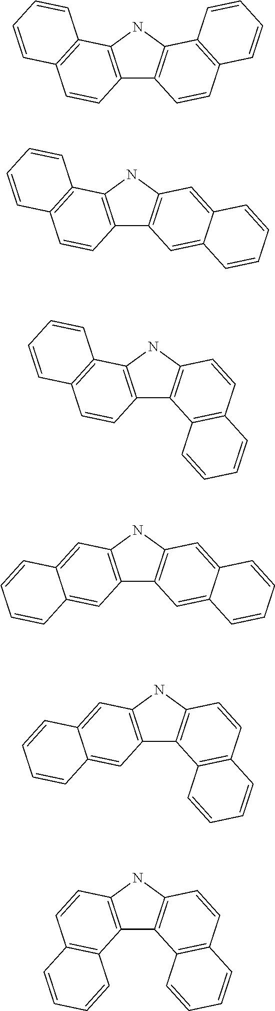 [SMILES: c1ccc2c(c1)ccc1c3ccc4ccccc4c3[nH]c21 ; c1ccc2cc3c(cc2c1)[nH]c1c2ccccc2ccc31 ; c1ccc2c(c1)ccc1c2[nH]c2ccc3ccccc3c21 ; c1ccc2cc3c(cc2c1)[nH]c1cc2ccccc2cc13 ; c1ccc2cc3c(cc2c1)[nH]c1ccc2ccccc2c13 ; c1ccc2c(c1)ccc1[nH]c3ccc4ccccc4c3c12]

In the present specification, when the substituent is a carbazole group, a benzocarbazole group, or a dibenzocarbazole group, it means being bonded to the nitrogen or carbon of the carbazole group, the benzocarbazole group, or the dibenzocarbazole group.

In the present specification, when a carbazole group, a benzocarbazole group, or a dibenzocarbazole group is substituted, an additional substituent may be substituted at the nitrogen or carbon of the carbazole group, the benzocarbazole group, or the dibenzocarbazole group.

In the present specification, a naphthobenzofuran group may be any one of the following structures.

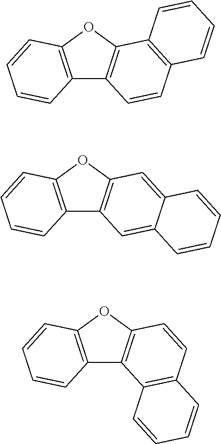 [SMILES: c1ccc2c(c1)ccc1c3ccccc3oc21 ; c1ccc2cc3c(cc2c1)oc1ccccc13 ; c1ccc2c(c1)ccc1oc3ccccc3c12]

In the present specification, a naphthobenzothiophene group may be any one of the following structures.

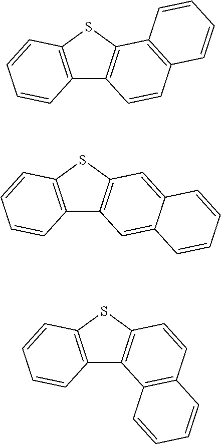 [SMILES: c1ccc2c(c1)ccc1c3ccccc3sc21 ; c1ccc2cc3c(cc2c1)sc1ccccc13 ; c1ccc2c(c1)ccc1sc3ccccc3c12]

In the present specification, a silyl group includes Si and is a substituent to which the Si atom is directly linked as a radical, and is represented by —Si(R107)(R108)(R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

Specific examples of the silyl group include the following structures, but are not limited thereto.

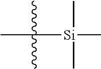 [SMILES: *[Si](C)(C)C]

(A trimethylsilyl group),

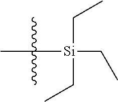 [SMILES: *[Si](CC)(CC)CC]

(a triethylsilyl group),

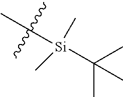 [SMILES: *[Si](C)(C)C(C)(C)C]

(a t-butyldimethylsilyl group),

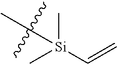 [SMILES: *[Si](C)(C)C=C]

(a vinyldimethylsilyl group),

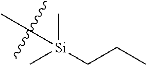 [SMILES: *[Si](C)(C)CCC]

(a propyldimethylsilyl group),

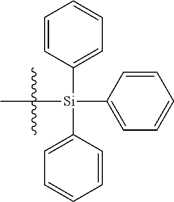 [SMILES: *[Si](c1ccccc1)(c1ccccc1)c1ccccc1]

(a triphehylsilyl group),

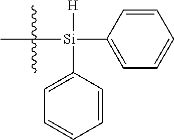 [SMILES: *[SiH](c1ccccc1)c1ccccc1]

(a diphenylsilyl group), and

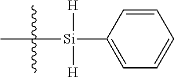 [SMILES: *[SiH2]c1ccccc1]

(a phenylsilyl group)

In the present specification, a phosphine oxide group is represented by —P(═O)(R110)(R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, an amine group is represented by —N(R112)(R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described description of the aryl group may be applied to an arylene group except for a divalent arylene group.

In the present specification, the above-described description of the heteroaryl group may be applied to a heteroarylene group except for a divalent heteroarylene group.

An exemplary embodiment of the present specification provides the heterocyclic compound represented by Chemical Formula 1.

In an exemplary embodiment of the present specification, R1 is hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R1 is hydrogen; deuterium; a halogen group; a cyano group; a substituted or unsubstituted C1 to C30 alkyl group; a substituted or unsubstituted C3 to C30 cycloalkyl group; a substituted or unsubstituted C2 to C30 heterocycloalkyl group; a substituted or unsubstituted C6 to C30 aryl group; or a substituted or unsubstituted C2 to C30 heteroaryl group.

In an exemplary embodiment of the present specification, R1 is hydrogen; deuterium; a substituted or unsubstituted C1 to C30 alkyl group; a substituted or unsubstituted C6 to C30 aryl group; or a substituted or unsubstituted C2 to C30 heteroaryl group.

In an exemplary embodiment of the present specification, R1 may be hydrogen; or deuterium.

In an exemplary embodiment of the present specification, Chemical Formula 1 may be represented by any one of the following Chemical Formulae 1-1 to 1-4.

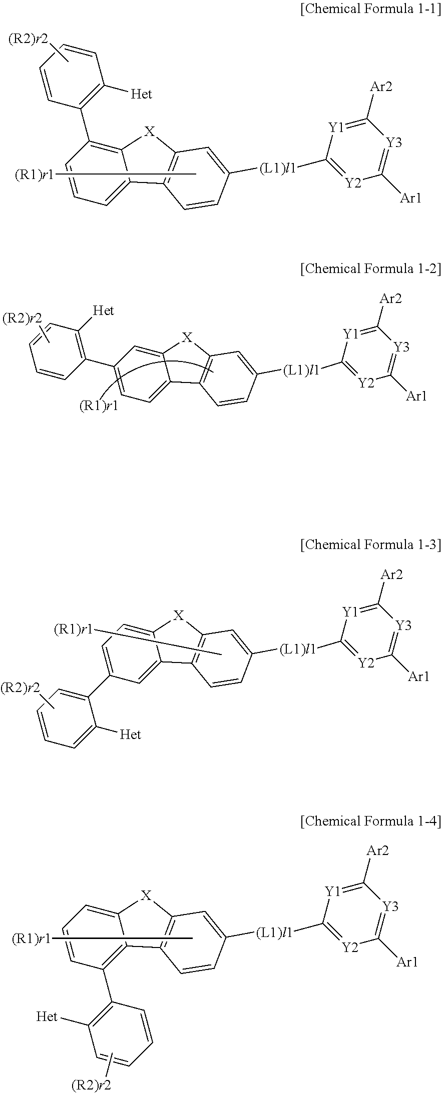

In Chemical Formulae 1-1 to 1-4, the definition of each substituent is the same as that in Chemical Formula 1.

In an exemplary embodiment of the present specification, L1 is a direct bond; a substituted or unsubstituted C6 to C60 arylene group; or a substituted or unsubstituted C2 to C60 heteroarylene group.

In an exemplary embodiment of the present specification, L1 may be a direct bond; a substituted or unsubstituted C6 to C30 arylene group; or a substituted or unsubstituted C2 to C30 heteroarylene group.

In an exemplary embodiment of the present specification, L1 may be a direct bond; or a substituted or unsubstituted C6 to C30 arylene group.

In an exemplary embodiment of the present specification, L1 may be a direct bond; a substituted or unsubstituted phenylene group; or a substituted unsubstituted biphenylene group.

In an exemplary embodiment of the present specification, L1 may be a direct bond; or a C6 to C30 arylene group unsubstituted or substituted with deuterium.

In an exemplary embodiment of the present specification, L1 may be a direct bond; a phenylene group unsubstituted or substituted with deuterium; or a biphenylene group unsubstituted or substituted with deuterium.

In an exemplary embodiment of the present specification, Y1 to Y3 are each independently N or CR, and at least one thereof is N.

In an exemplary embodiment of the present specification, Y1 to Y3 are each independently N or CR, and at least two thereof are N.

In an exemplary embodiment of the present specification, Y1 to Y3 may be N.

In an exemplary embodiment of the present specification, Chemical Formula 1 may be represented by the following Chemical Formula 1-A.

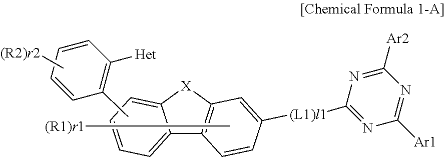

In Chemical Formula 1-A, the definition of each substituent is the same as that in Chemical Formula 1.

In an exemplary embodiment of the present specification, Ar1 and Ar2 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,

In an exemplary embodiment of the present specification, Ar1 and Ar2 are each independently hydrogen; deuterium; a halogen group; a cyano group; a substituted or unsubstituted C1 to C30 alkyl group; a substituted or unsubstituted C3 to C30 cycloalkyl group; a substituted or unsubstituted C2 to C30 heterocycloalkyl group; a substituted or unsubstituted C6 to C30 aryl group; or a substituted or unsubstituted C2 to C30 heteroaryl group.

In an exemplary embodiment of the present specification, Ar1 and Ar2 may be each independently a substituted or unsubstituted C6 to C30 aryl group; or a substituted or unsubstituted C2 to C30 heteroaryl group.

In an exemplary embodiment of the present specification, Ar1 and Ar2 may be each independently a substituted or unsubstituted phenyl group; a substituted or unsubstituted biphenyl group; or a substituted or unsubstituted dibenzofuran group.

In an exemplary embodiment of the present specification, Ar1 and Ar2 may be each independently a C6 to C30 aryl group unsubstituted or substituted with deuterium; or a C2 to C30 heteroaryl group unsubstituted or substituted with deuterium.

In an exemplary embodiment of the present specification, Ar1 and Ar2 may be each independently a phenyl group unsubstituted or substituted with deuterium; a biphenyl group unsubstituted or substituted with deuterium; or a dibenzofuran group unsubstituted or substituted with deuterium.

In an exemplary embodiment of the present specification, Het is a substituted or unsubstituted C2 to C60 fused polycyclic heteroaryl group.

In an exemplary embodiment of the present specification, Het may be a substituted or unsubstituted C2 to C30 fused polycyclic heteroaryl group.

In an exemplary embodiment of the present specification, Het may be a C2 to C30 fused polycyclic heteroaryl group which is substituted or unsubstituted and includes O, S, or N.

In an exemplary embodiment of the present specification, Het may be a substituted or unsubstituted carbazole group; a substituted or unsubstituted dibenzofuran group; or a substituted or unsubstituted dibenzothiophene group.

In an exemplary embodiment of the present specification, Het may be represented by the following Chemical Formula H-1 or H-2.

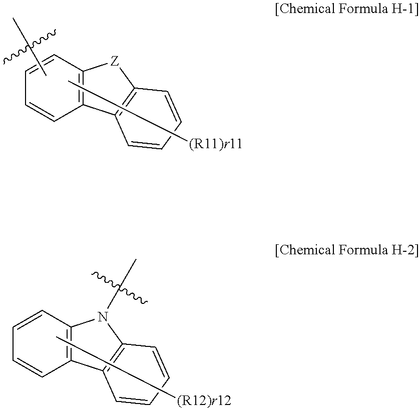

In Chemical Formulae H-1 and H-2,
Z is O; S; or NR′,
R′, R11, and R12 are each independently hydrogen; deuterium; a substituted or unsubstituted C1 to C30 alkyl group; a substituted or unsubstituted C6 to C30 aryl group; or a substituted or unsubstituted C2 to C30 heteroaryl group,
r11 is an integer from 0 to 7,
r12 is an integer from 0 to 8, and
when r11 and r12 are each 2 or higher, substituents in the parenthesis are the same as or different from each other.

In an exemplary embodiment of the present specification, Z may be O.

In an exemplary embodiment of the present specification, Z may be S.

In an exemplary embodiment of the present specification, Z is NR′, and R′ may be a substituted or unsubstituted C6 to C30 aryl group.

In an exemplary embodiment of the present specification, R′ may be a substituted or unsubstituted phenyl group.

In an exemplary embodiment of the present specification, R′ may be a C6 to C30 aryl group unsubstituted or substituted with deuterium.

In an exemplary embodiment of the present specification, R′ may be a phenyl group unsubstituted or substituted with deuterium.

In an exemplary embodiment of the present specification, R11 and R12 may be each independently hydrogen; deuterium; or a substituted or unsubstituted C6 to C30 aryl group.

In an exemplary embodiment of the present specification, R11 and R12 may be each independently hydrogen; deuterium; or a substituted or unsubstituted phenyl group.

In an exemplary embodiment of the present specification, R11 and R12 may be each independently hydrogen; deuterium; or a C6 to C30 aryl group unsubstituted or substituted with deuterium.

In an exemplary embodiment of the present specification, R11 and R12 may be each independently hydrogen; deuterium; or a phenyl group unsubstituted or substituted with deuterium.

In an exemplary embodiment of the present specification, the deuterium content of Chemical Formula 1 may be 0% to 100%.

In an exemplary embodiment of the present specification, the deuterium content of Chemical Formula 1 may be 0%.

In an exemplary embodiment of the present specification, the deuterium content of Chemical Formula 1 may be 20% to 100%.

In an exemplary embodiment of the present specification, the deuterium content of Chemical Formula 1 may be 30% to 100%.

In an exemplary embodiment of the present specification, the deuterium content of Chemical Formula 1 may be 50% to 100%.

In an exemplary embodiment of the present specification, the deuterium content of Chemical Formula 1 may be 70% to 100%.

In an exemplary embodiment of the present specification, the deuterium content of Chemical Formula 1 may be 100%.

In an exemplary embodiment of the present specification, the heterocyclic compound of the Chemical Formula 1 satisfies the deuterium content in the above range, and the photochemical characteristics of a compound which includes deuterium and a compound which does not include deuterium are almost similar, but when deposited on a thin film, the deuterium-containing material tends to be packed with a narrower intermolecular distance.

Accordingly, when an electron only device (EOD) and a hole only device (HOD) are manufactured and the current density thereof according to voltage is confirmed, it can be confirmed that among the heterocyclic compounds of Chemical Formula 1 of the present invention, a compound including deuterium exhibits much more balanced charge transport characteristics than a compound which does not include deuterium.

Further, when the surface of a thin film is observed using an atomic force microscope (AFM), it can be confirmed that the thin film made of a compound including deuterium is deposited with a more uniform surface without any aggregated portion.

Additionally, since the single bond dissociation energy of carbon and deuterium is higher than the single bond dissociation energy of carbon and hydrogen, among the heterocyclic compounds of Chemical Formula 1 of the present invention, a compound in which the deuterium content satisfies the above range has the increased stability of the total molecules, so that there is an effect that the service life of the device is improved.

In an exemplary embodiment of the present specification, the deuterium content of Chemical Formula 1 may be 0%, or 20% to 100%.

In an exemplary embodiment of the present specification, the deuterium content of Chemical Formula 1 may be 0% or 30% to 100%.

In an exemplary embodiment of the present specification, the deuterium content of Chemical Formula 1 may be 0% or 50% to 100%.

In an exemplary embodiment of the present specification, the deuterium content of Chemical Formula 1 may be 0% or 70% to 100%.

In an exemplary embodiment of the present specification, the deuterium content of Chemical Formula 1 may be 0% or 100%.

In an exemplary embodiment of the present specification, Chemical Formula 1 may be represented by any one of the following compounds.

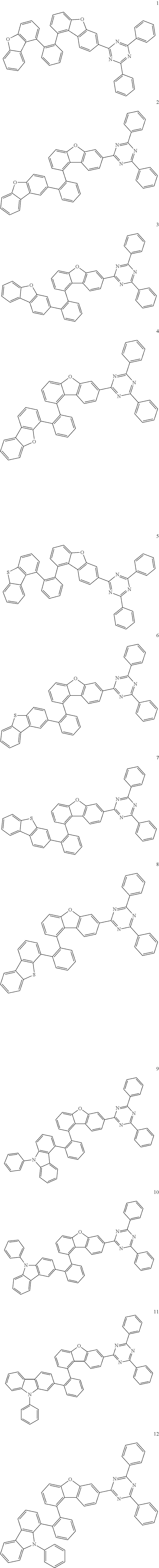

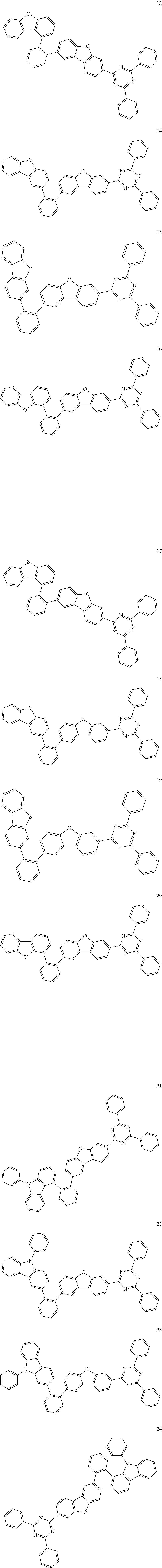

[chemical structure]

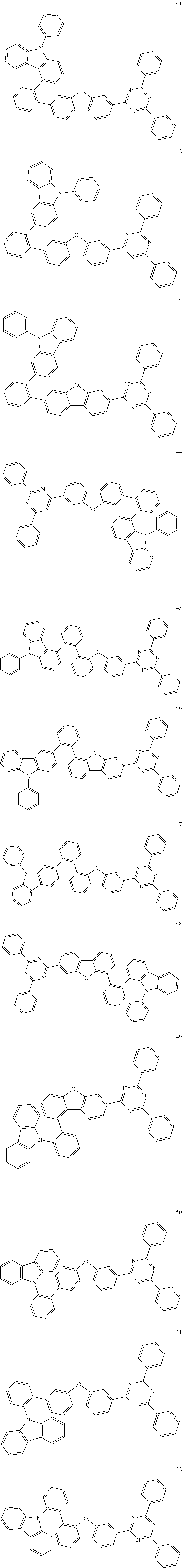

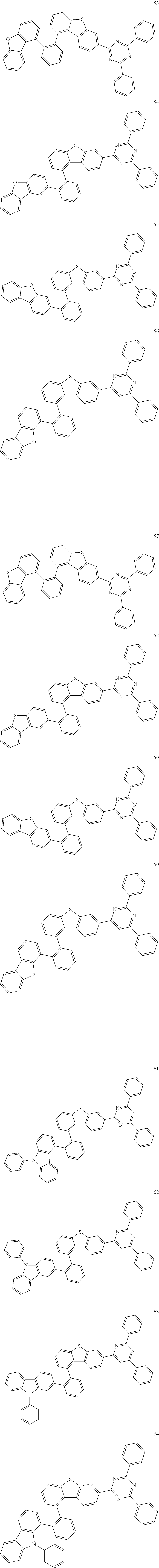

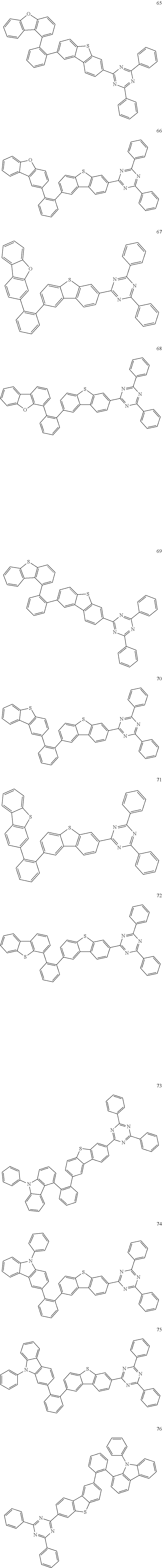

[chemical structure]

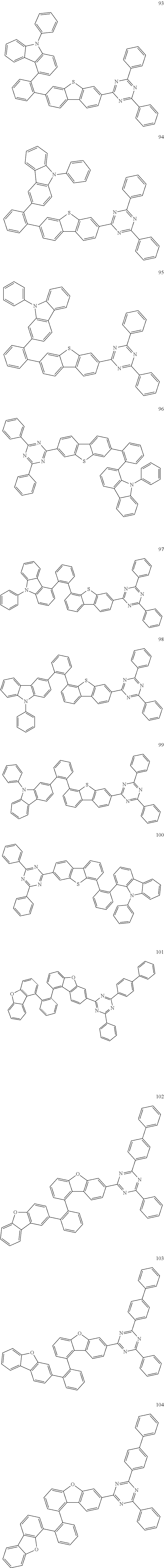

[chemical structure]

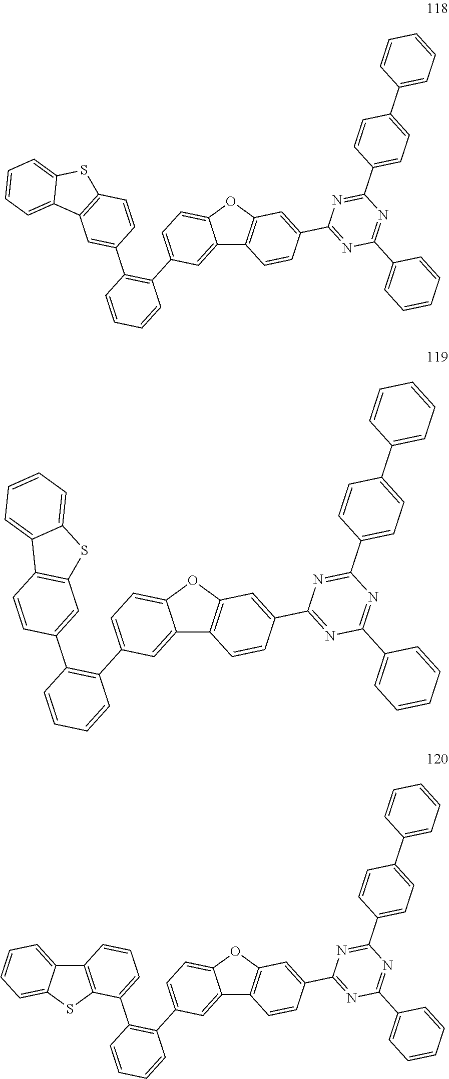 [SMILES: c1ccc(-c2ccc(-c3nc(-c4ccccc4)nc(-c4ccc5c(c4)oc4ccc(-c6ccccc6-c6ccc7sc8ccccc8c7c6)cc45)n3)cc2)cc1 ; c1ccc(-c2ccc(-c3nc(-c4ccccc4)nc(-c4ccc5c(c4)oc4ccc(-c6ccccc6-c6ccc7c(c6)sc6ccccc67)cc45)n3)cc2)cc1 ; c1ccc(-c2ccc(-c3nc(-c4ccccc4)nc(-c4ccc5c(c4)oc4ccc(-c6ccccc6-c6cccc7c6sc6ccccc67)cc45)n3)cc2)cc1]

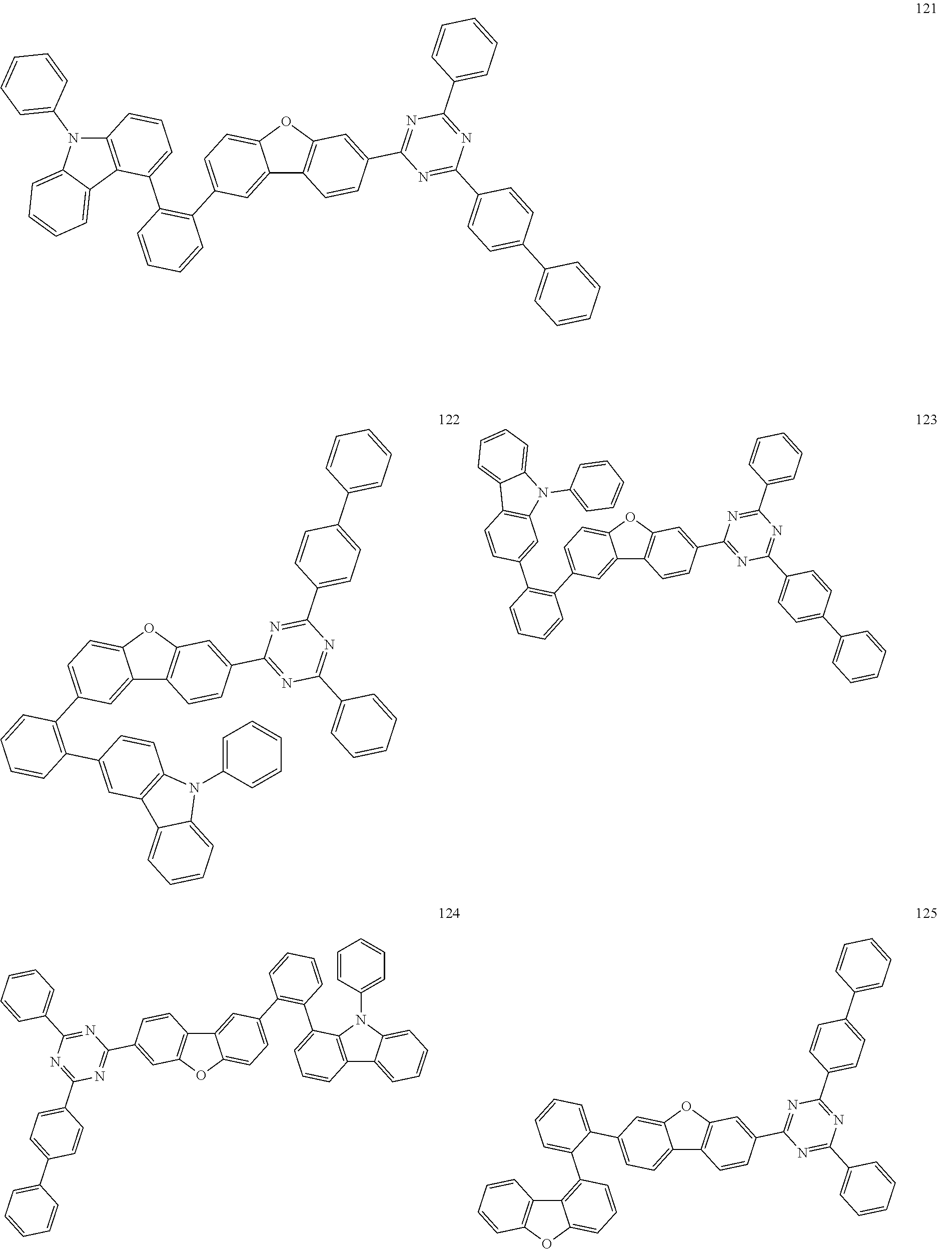

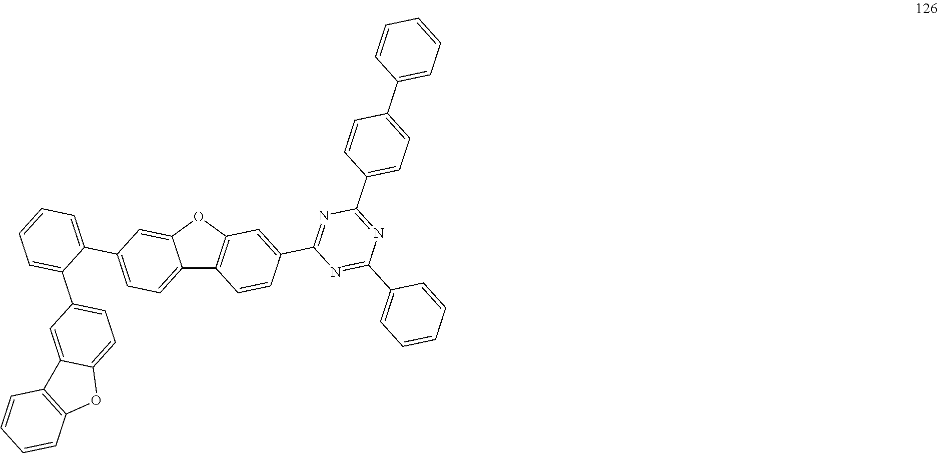 [SMILES: c1ccc(-c2ccc(-c3nc(-c4ccccc4)nc(-c4ccc5c(c4)oc4cc(-c6ccccc6-c6ccc7oc8ccccc8c7c6)ccc45)n3)cc2)cc1]

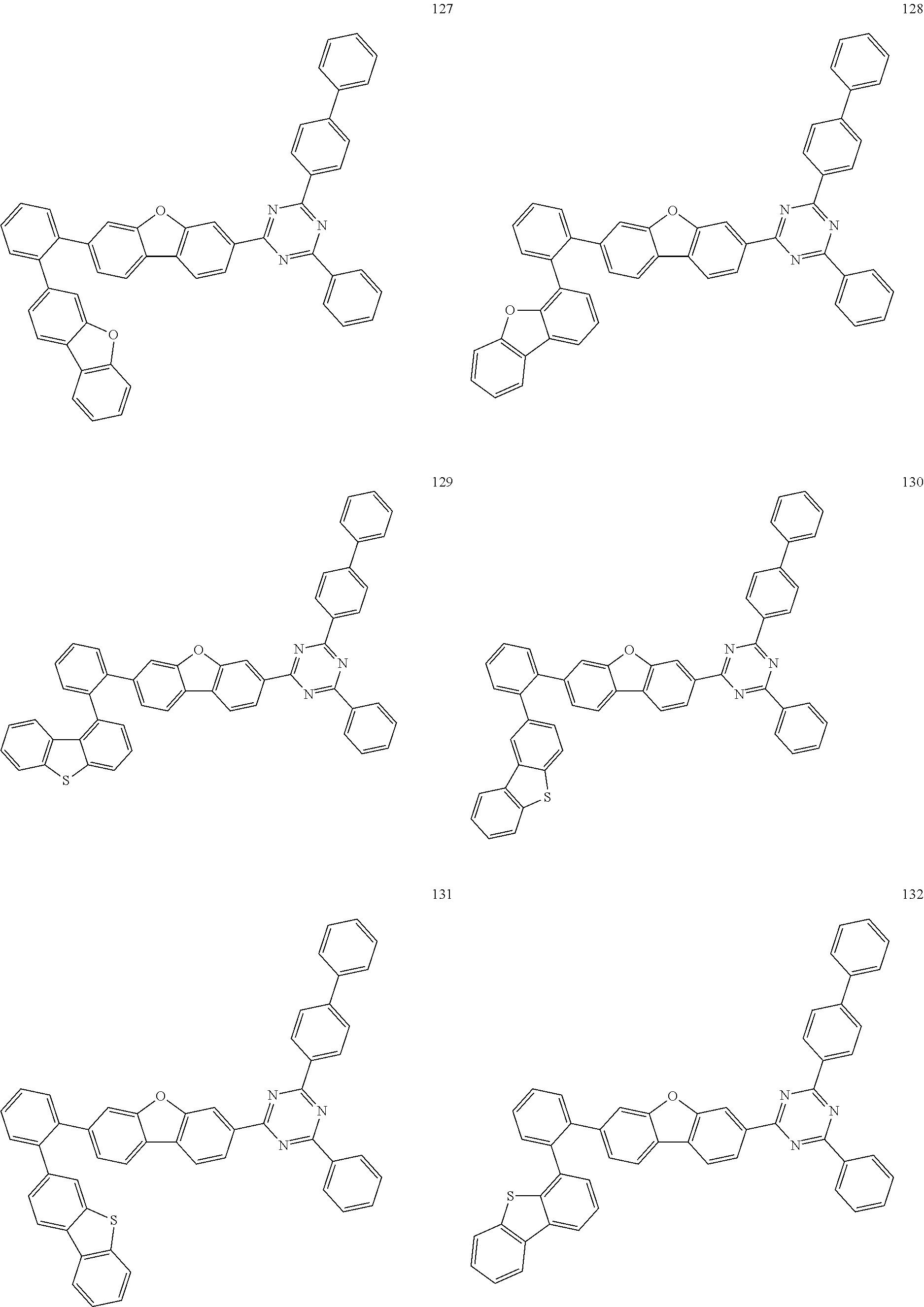

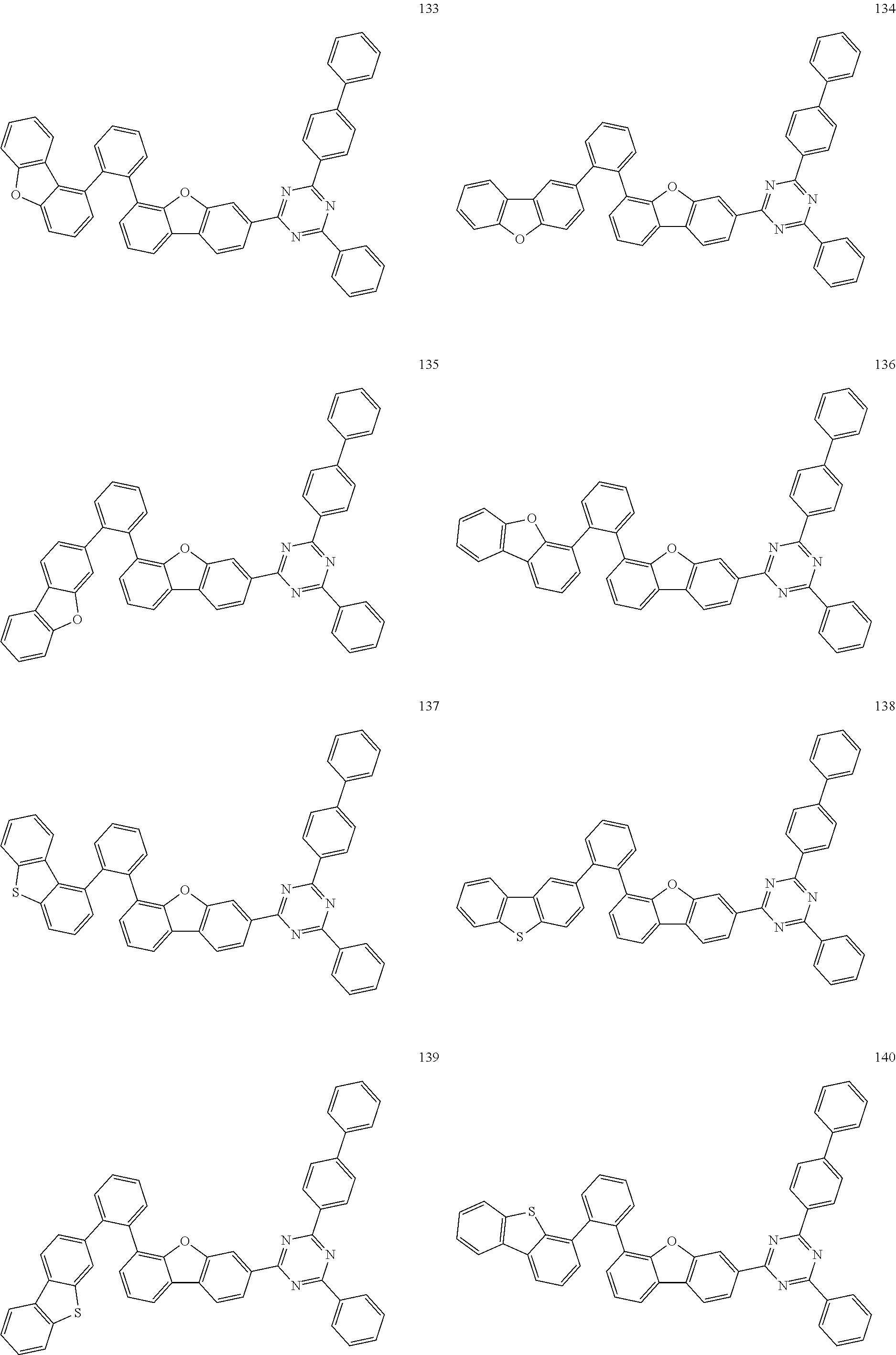

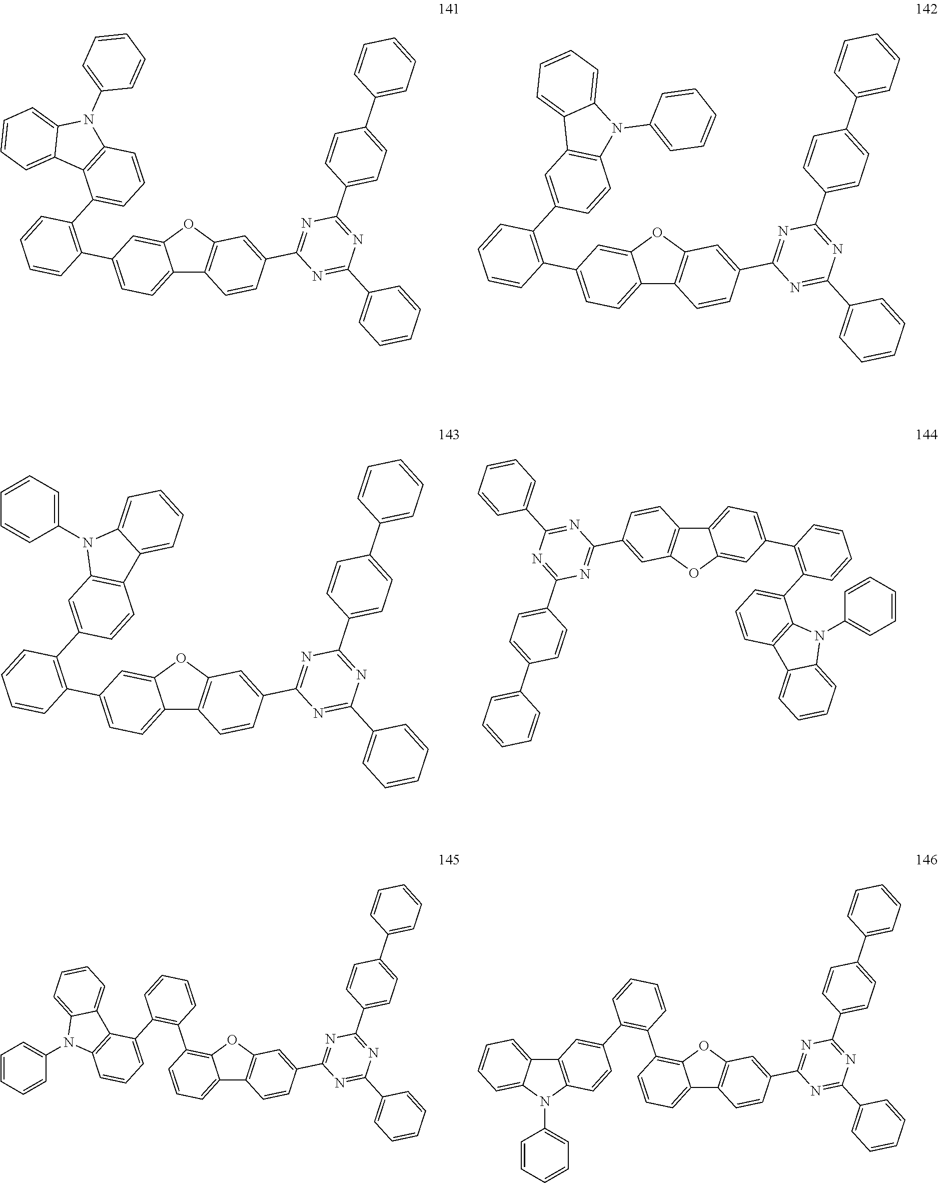

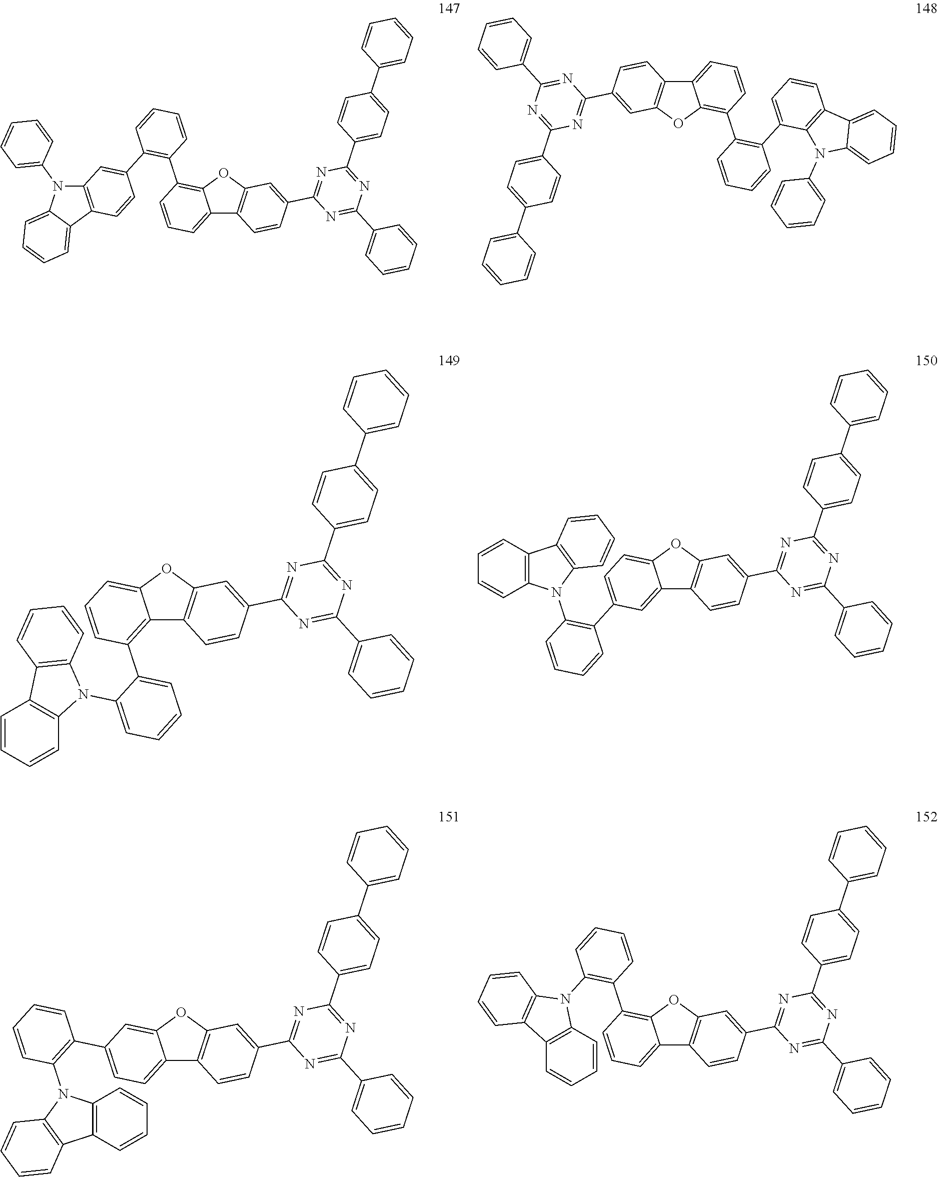

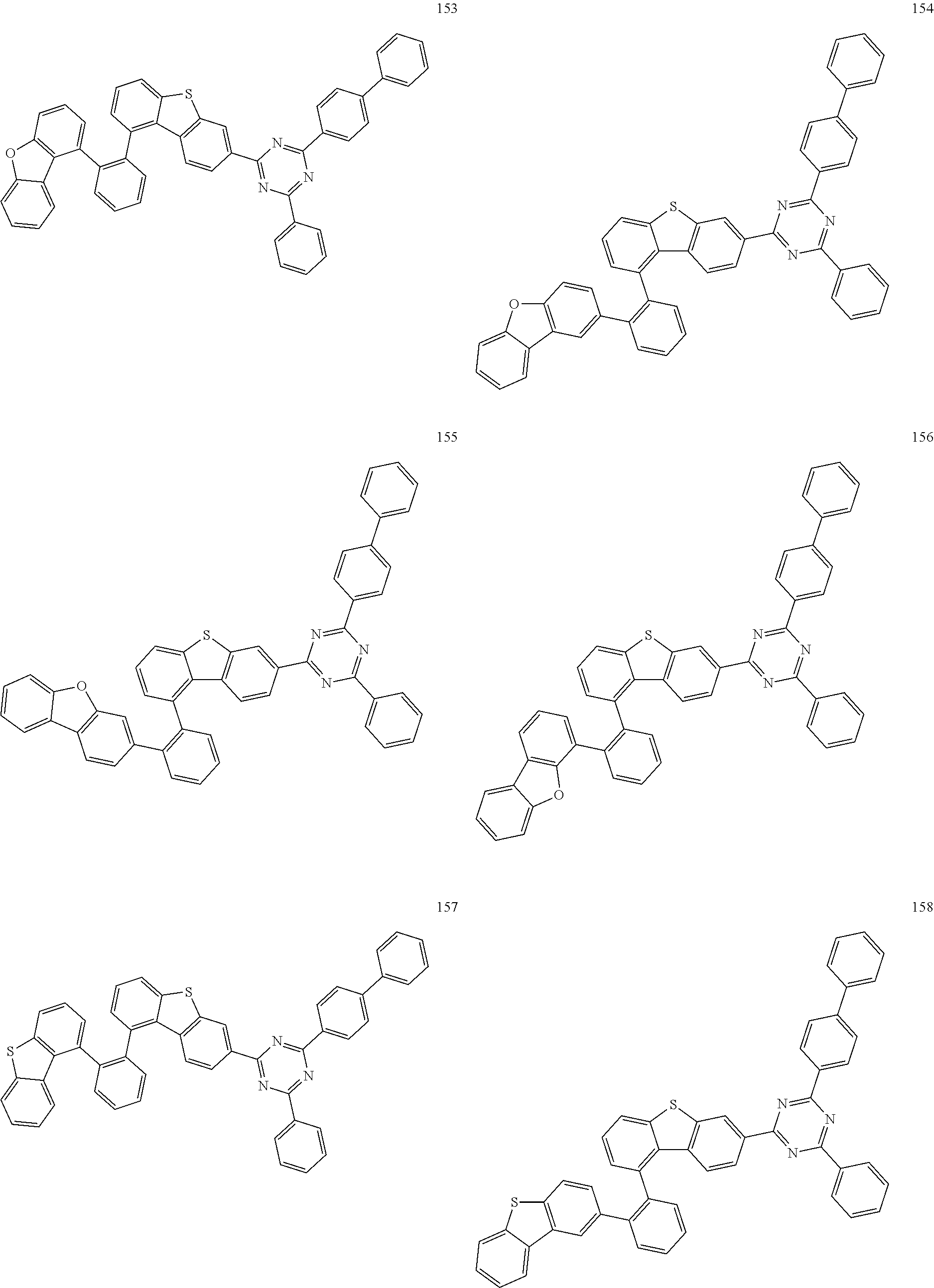

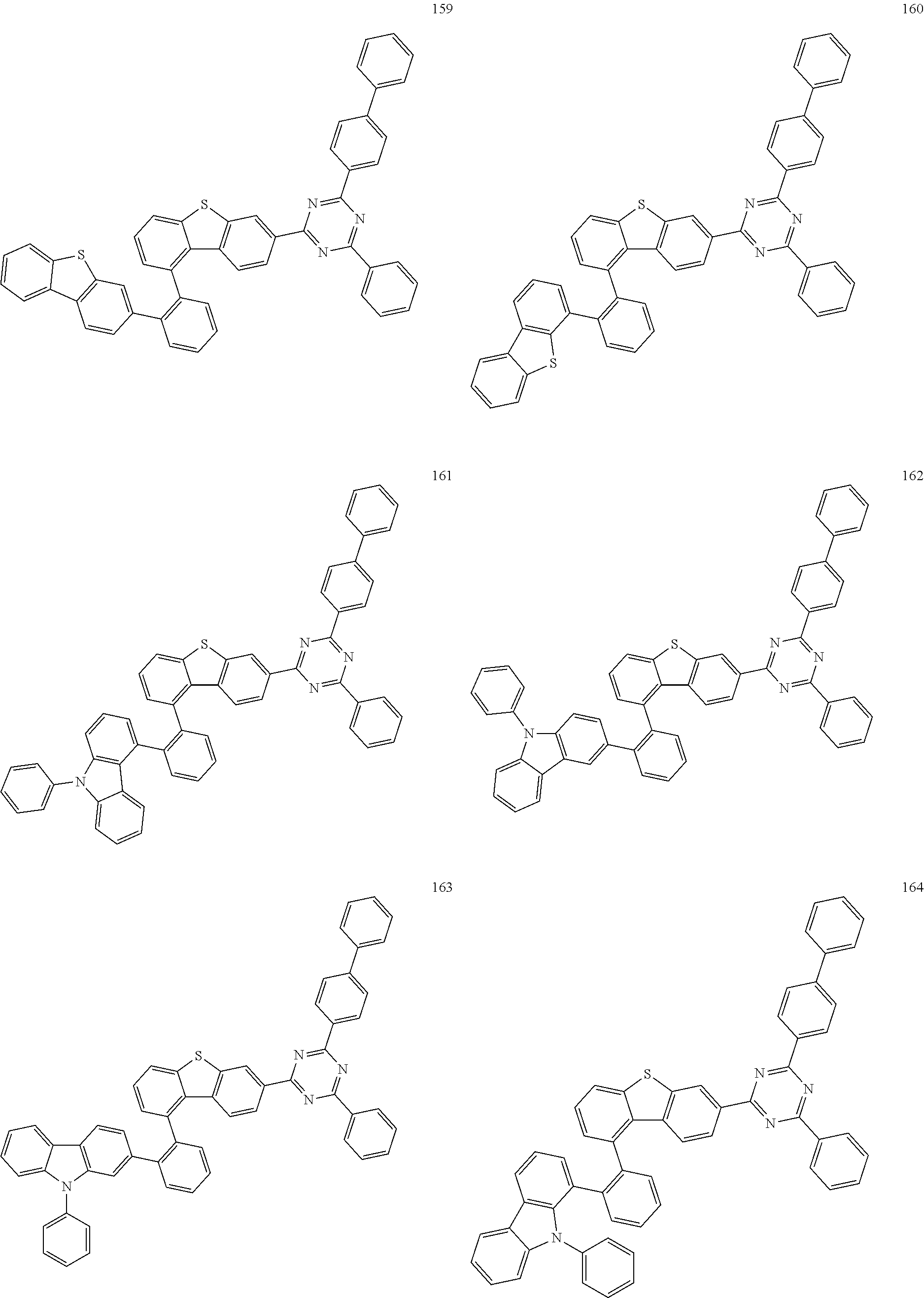

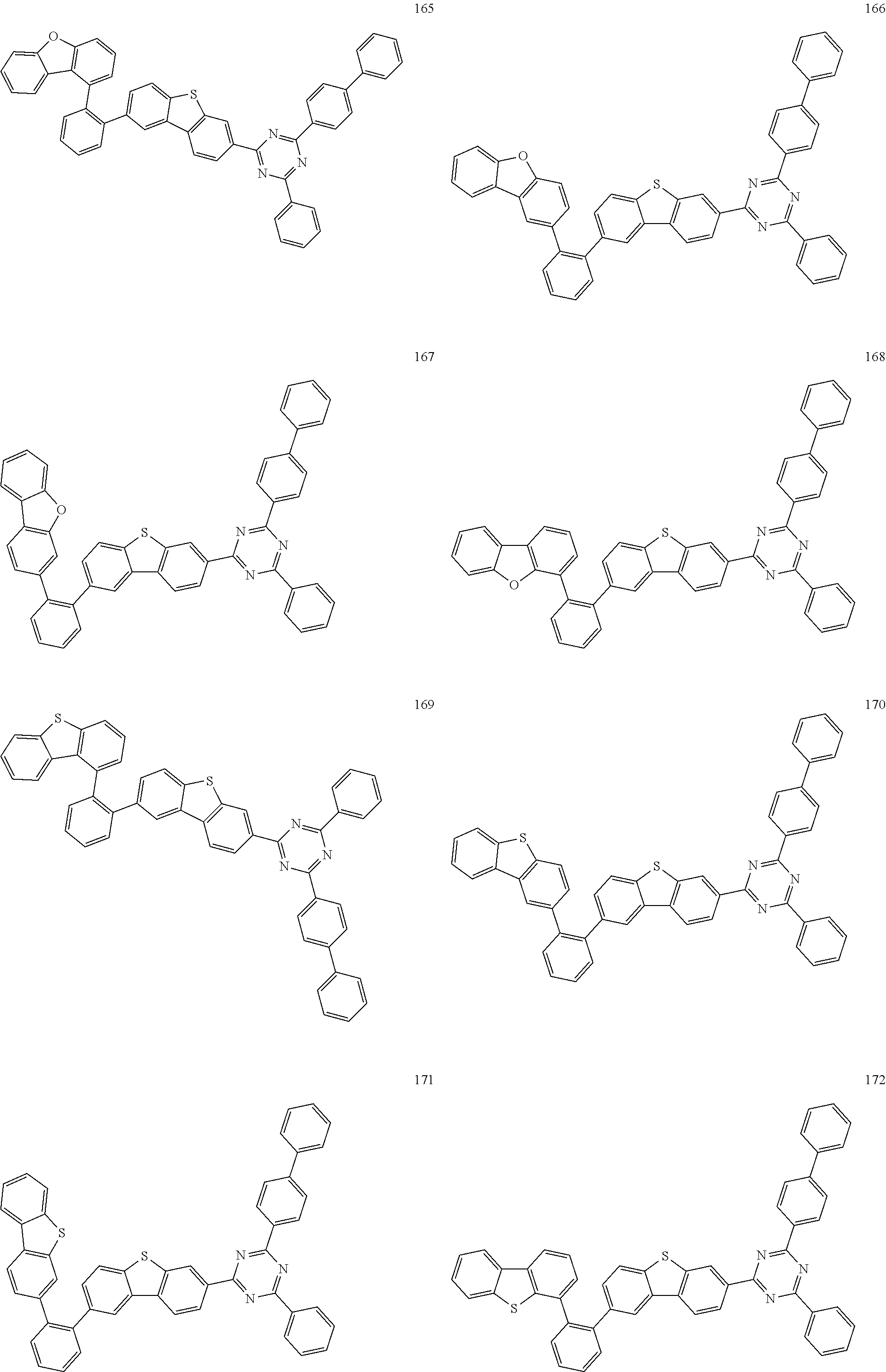

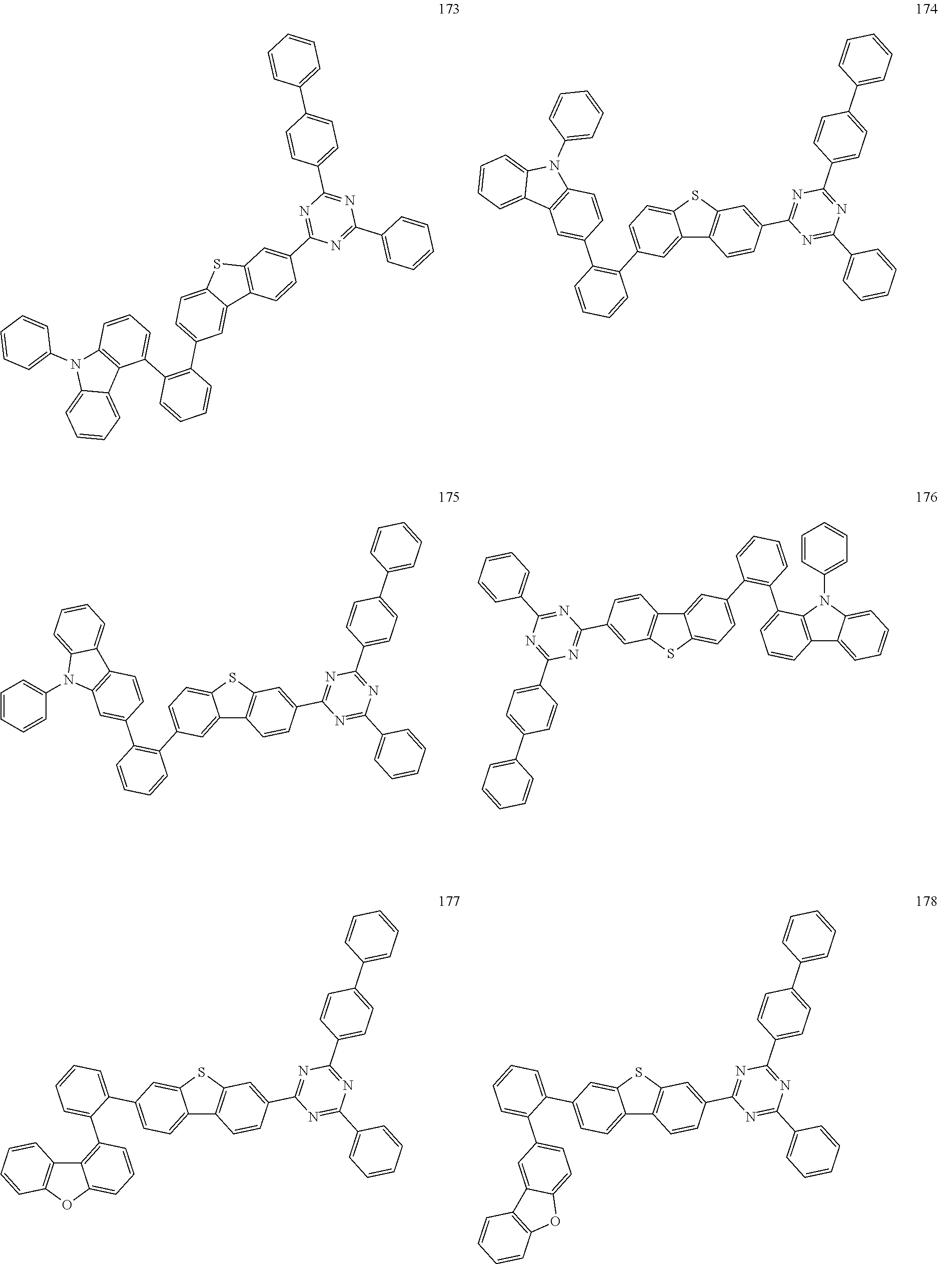

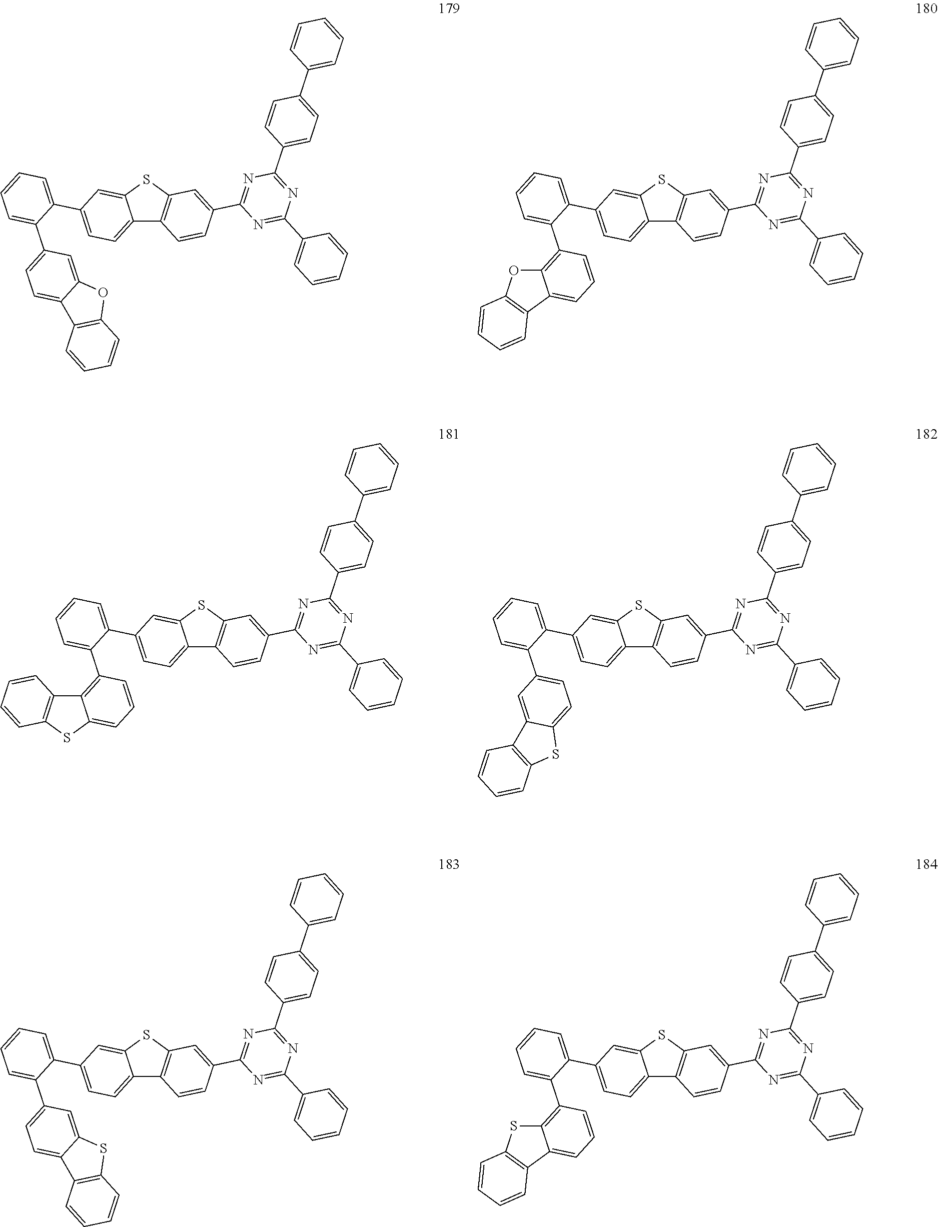

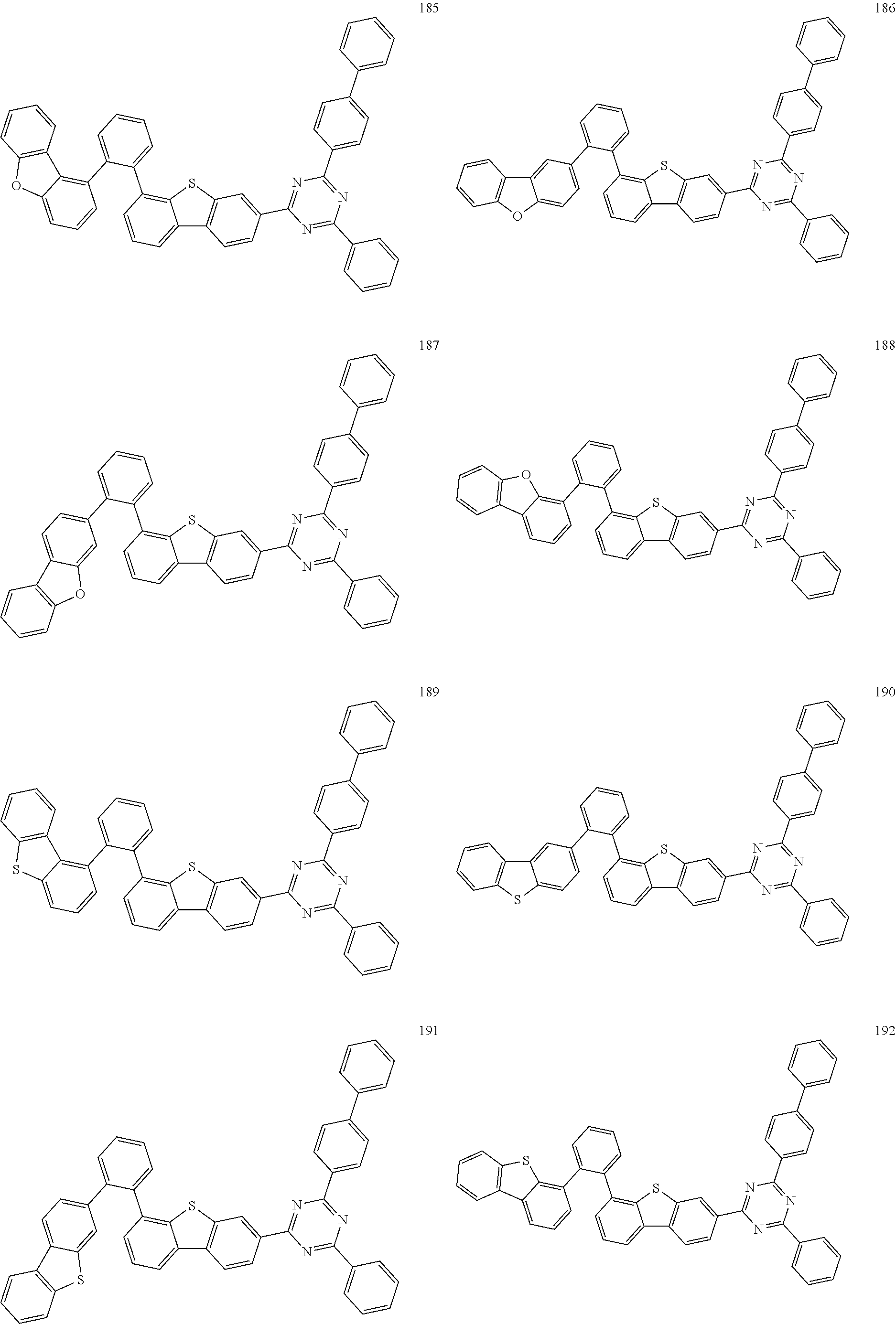

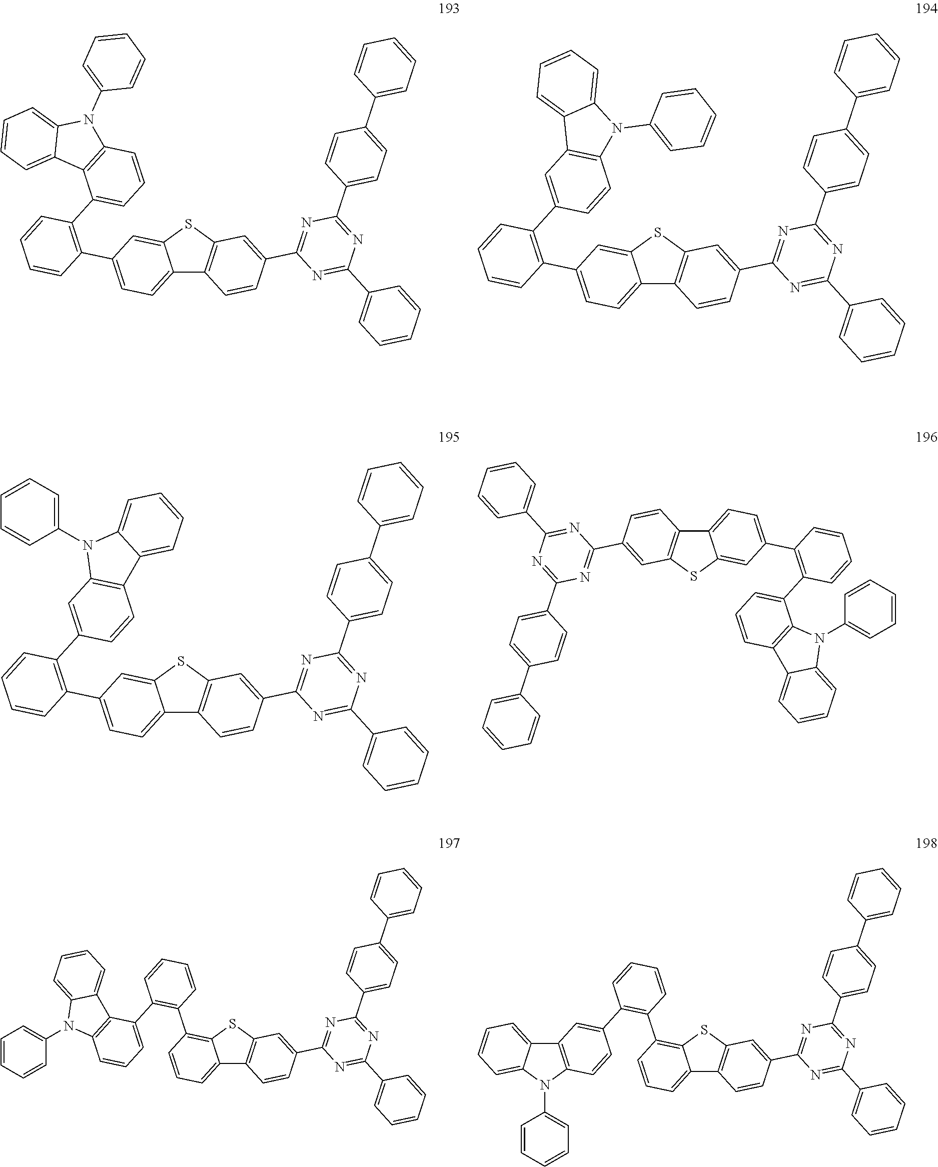

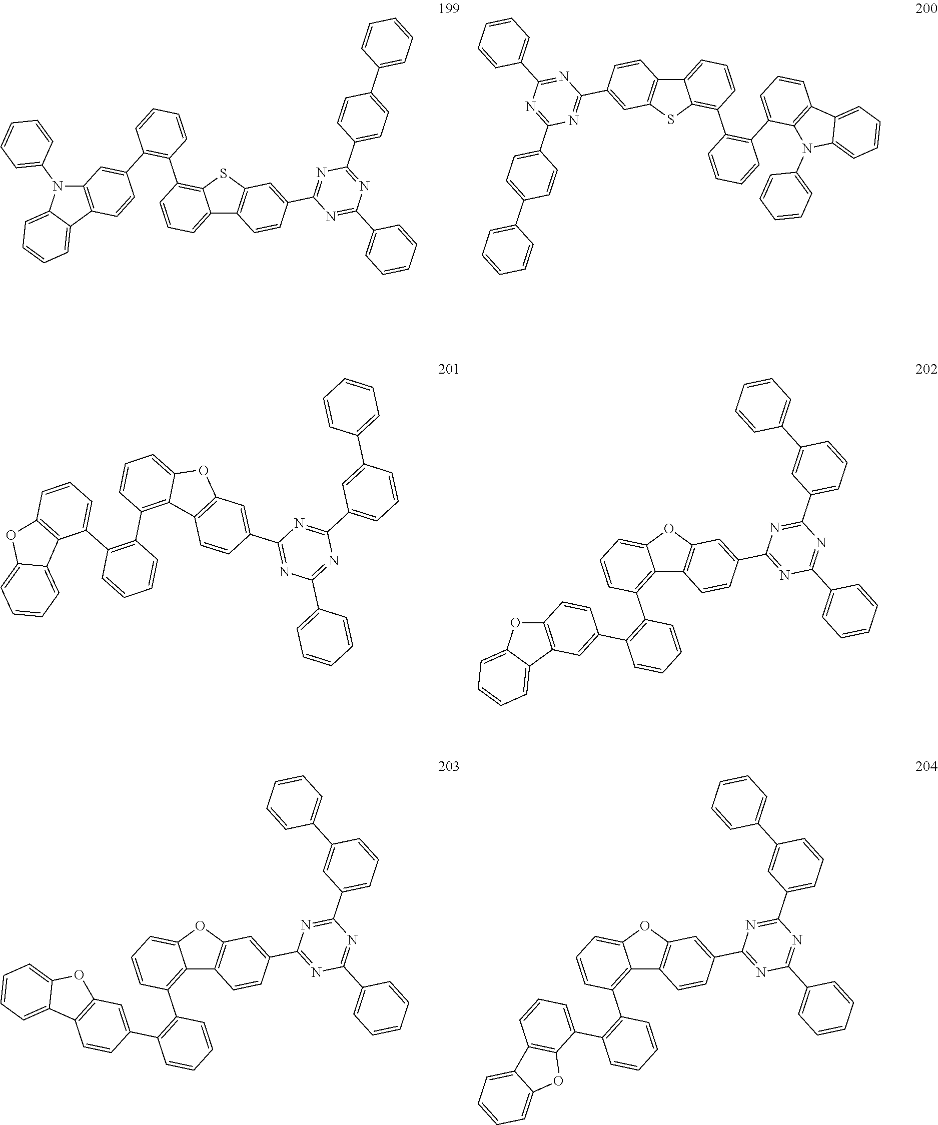

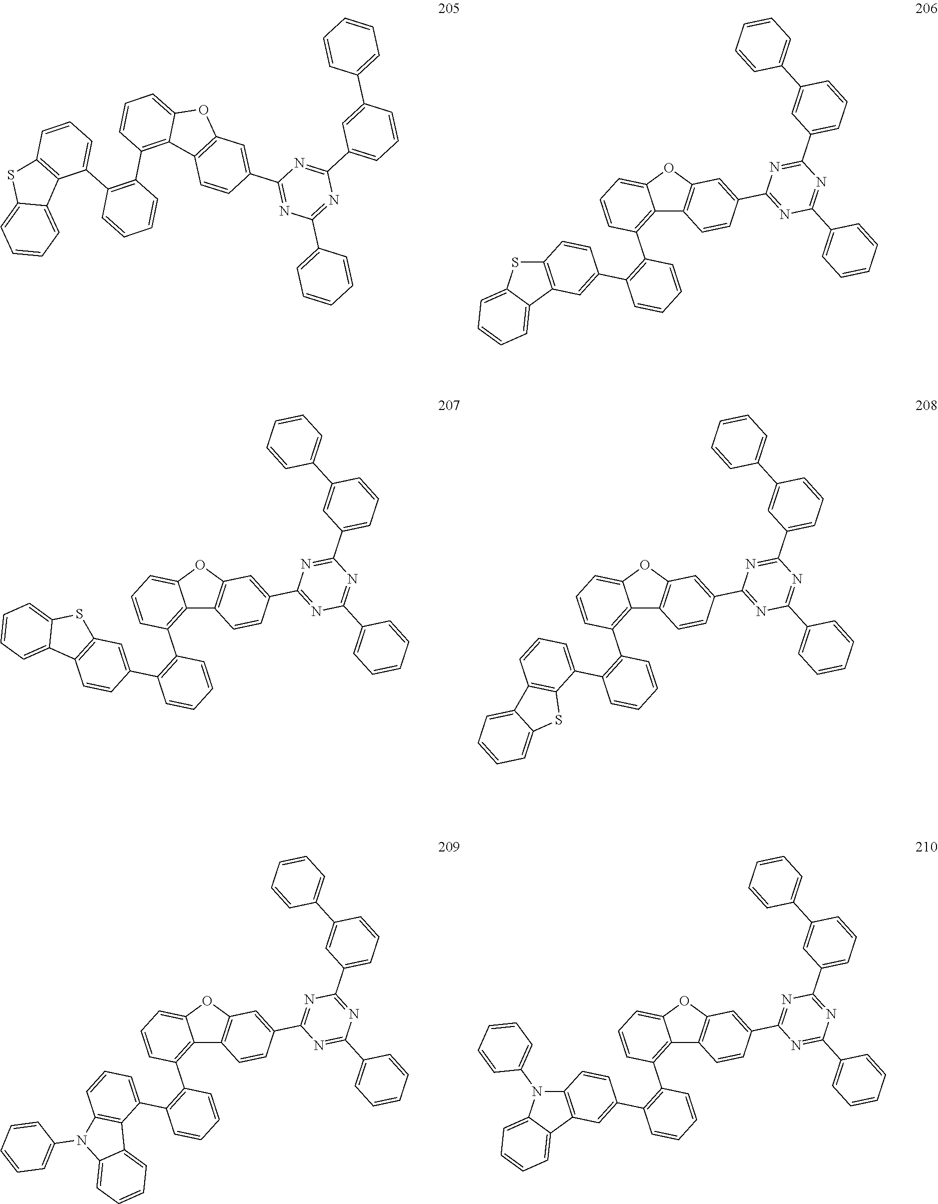

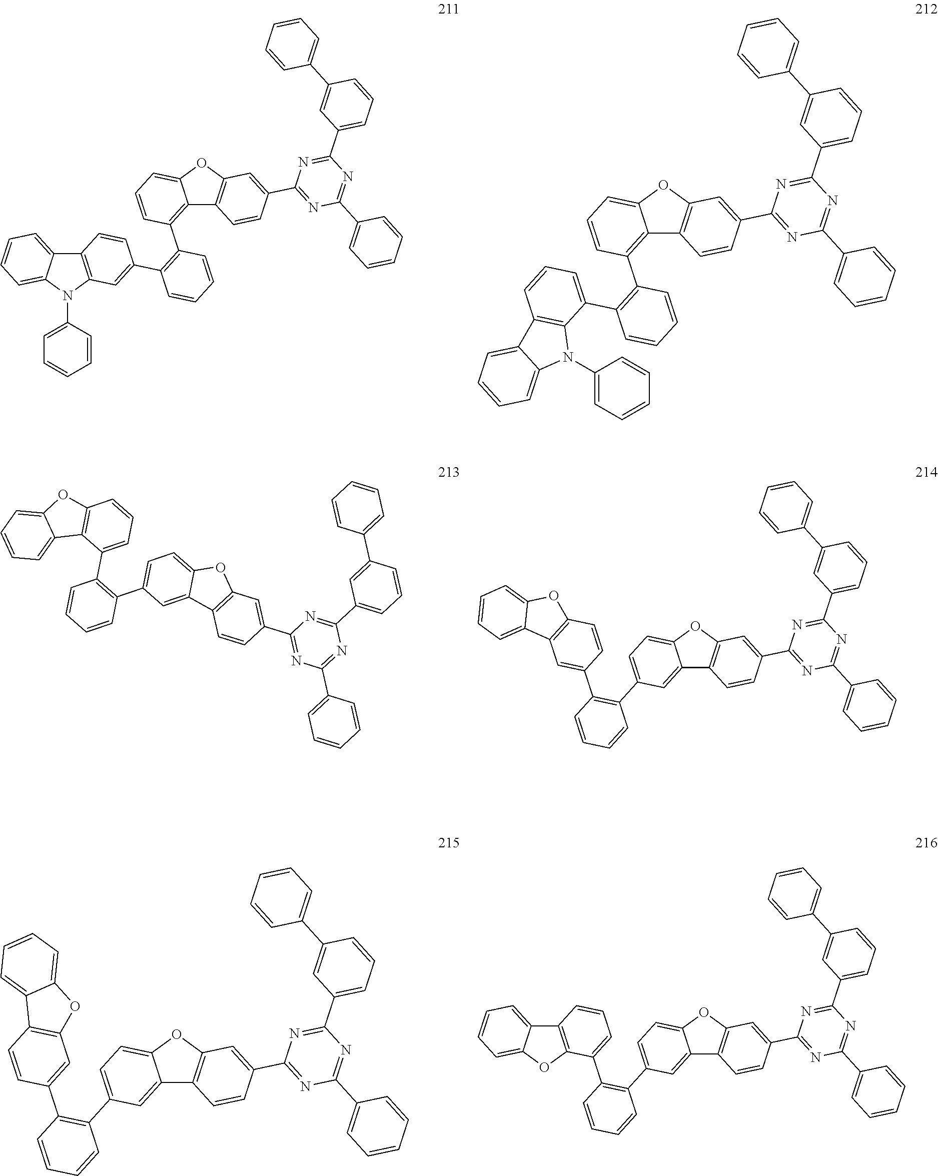

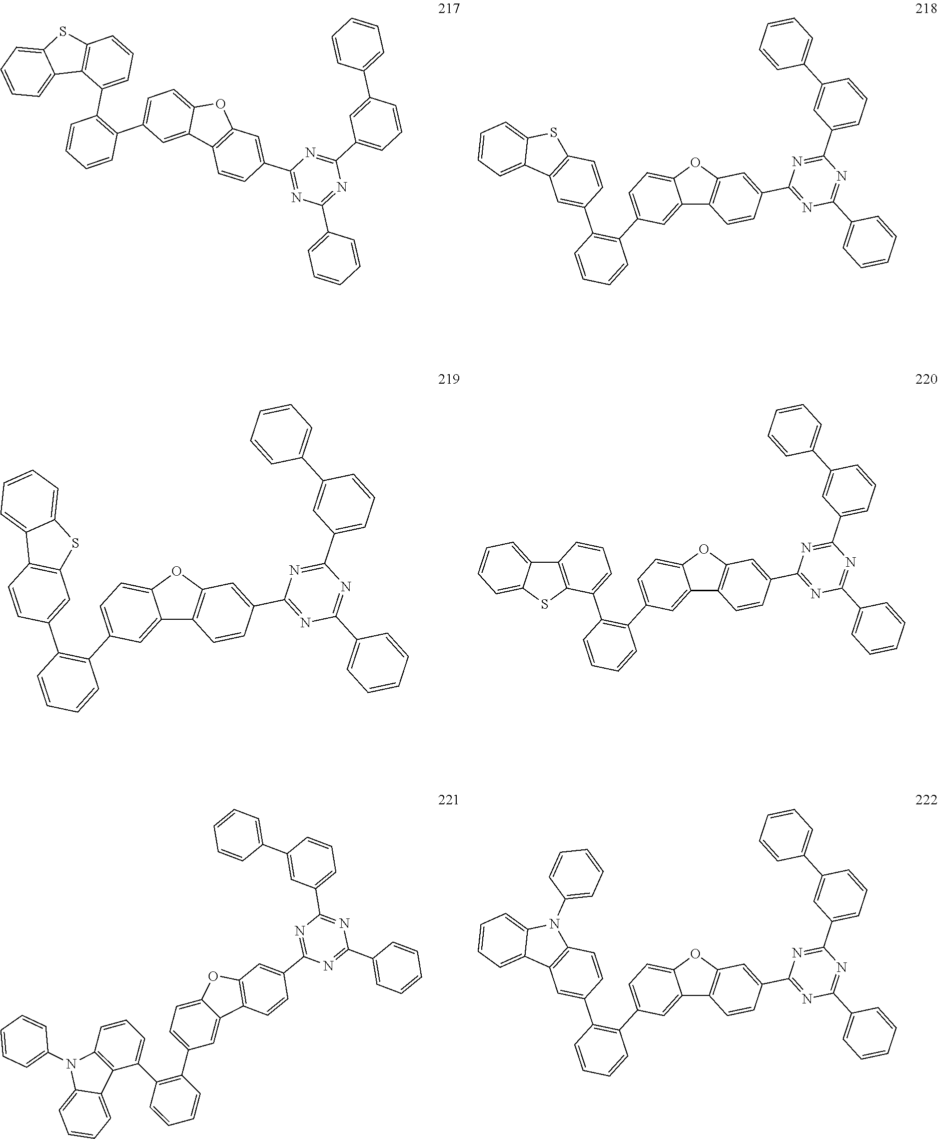

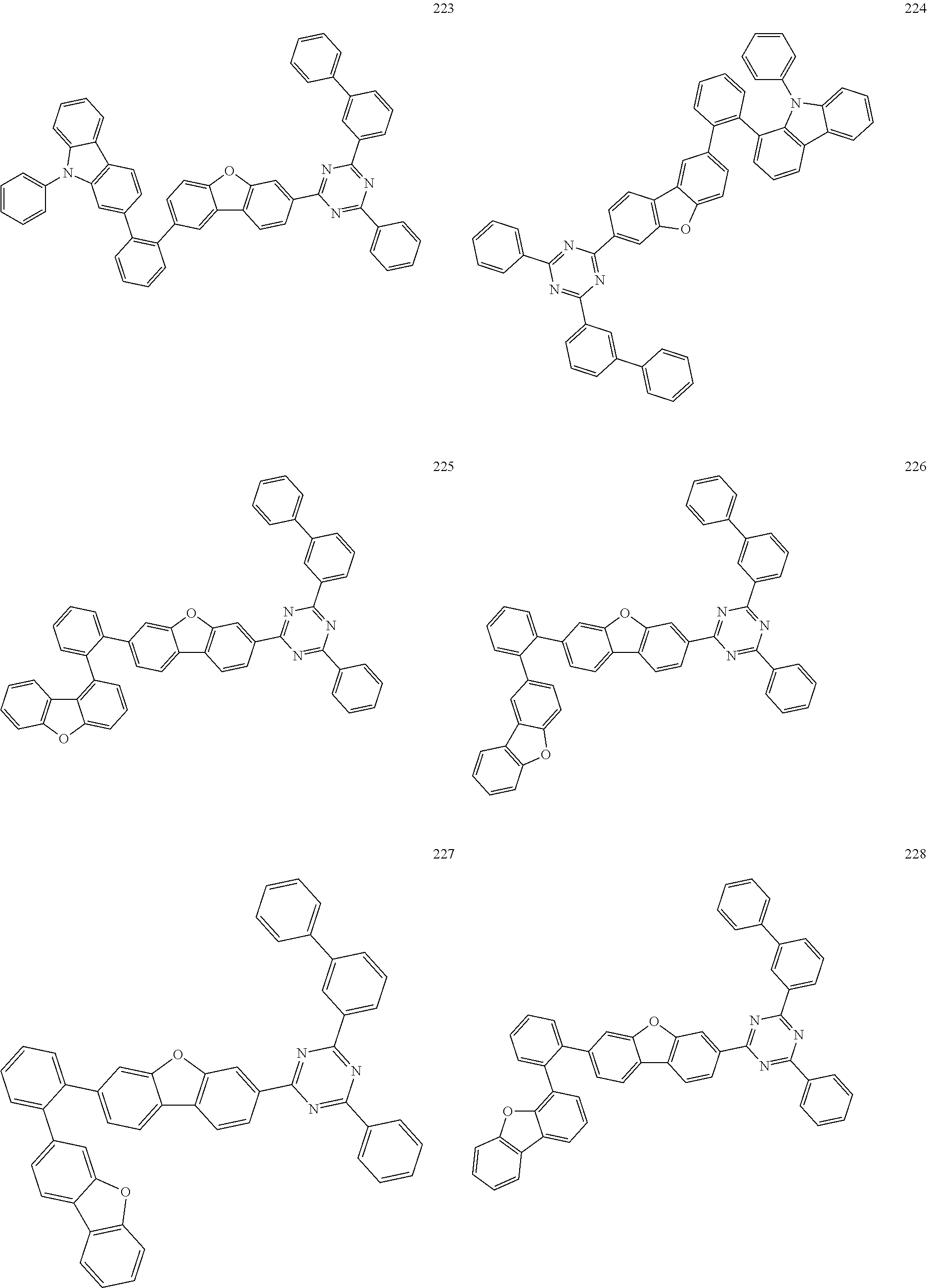

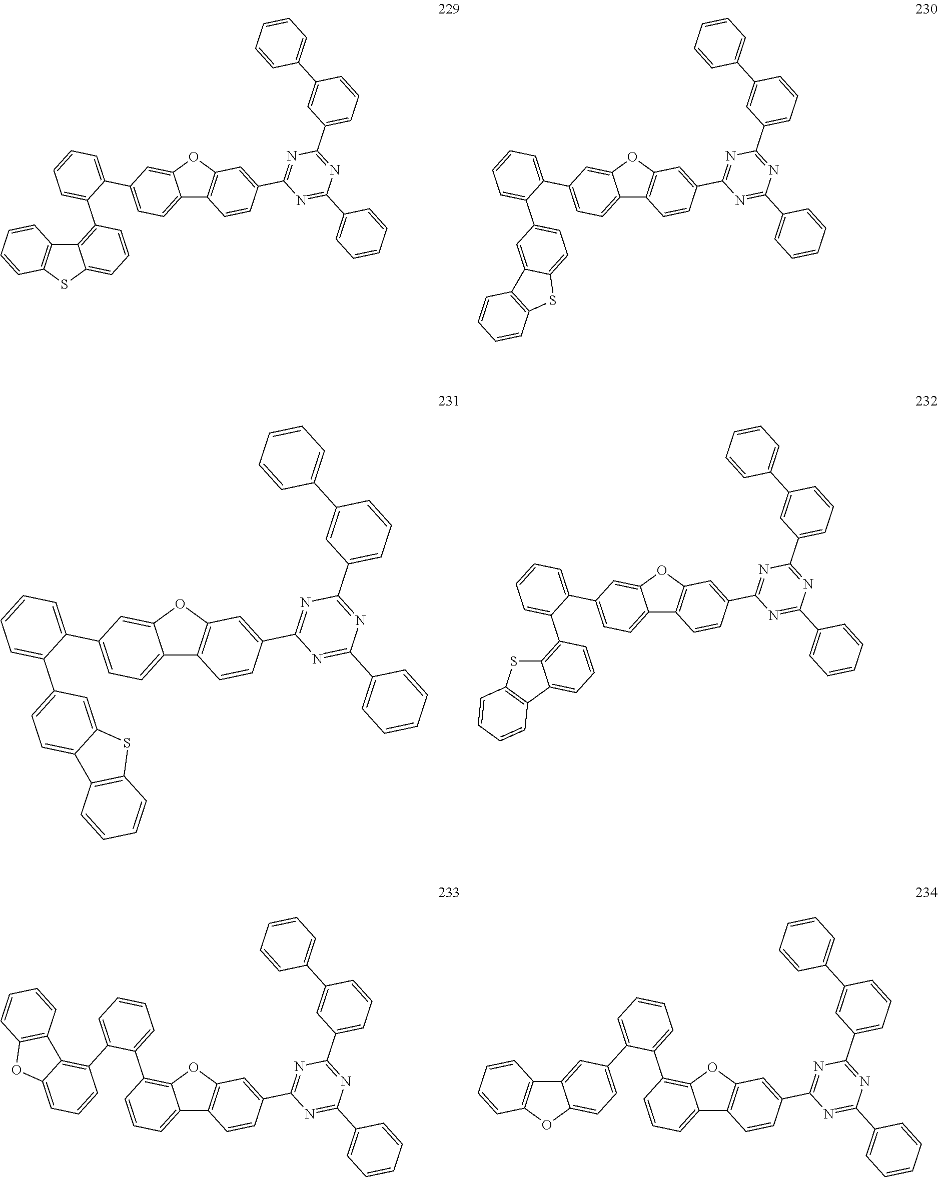

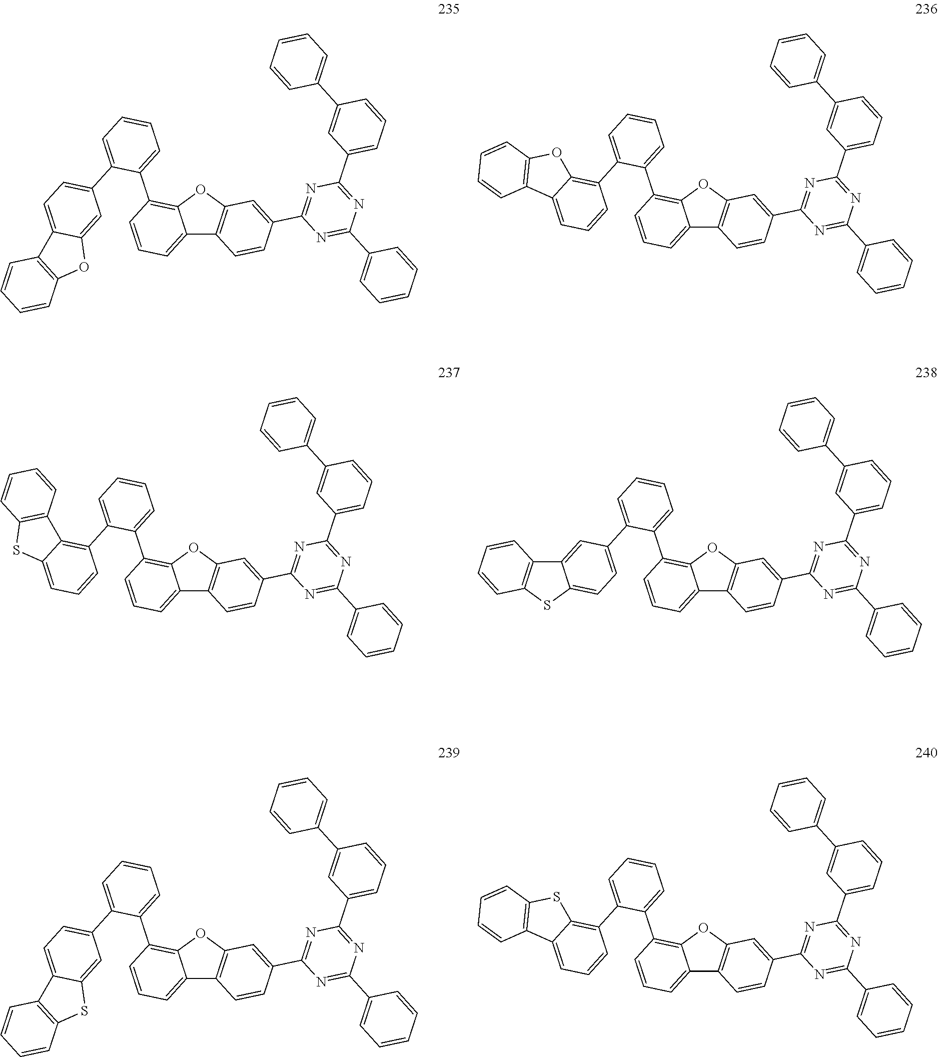

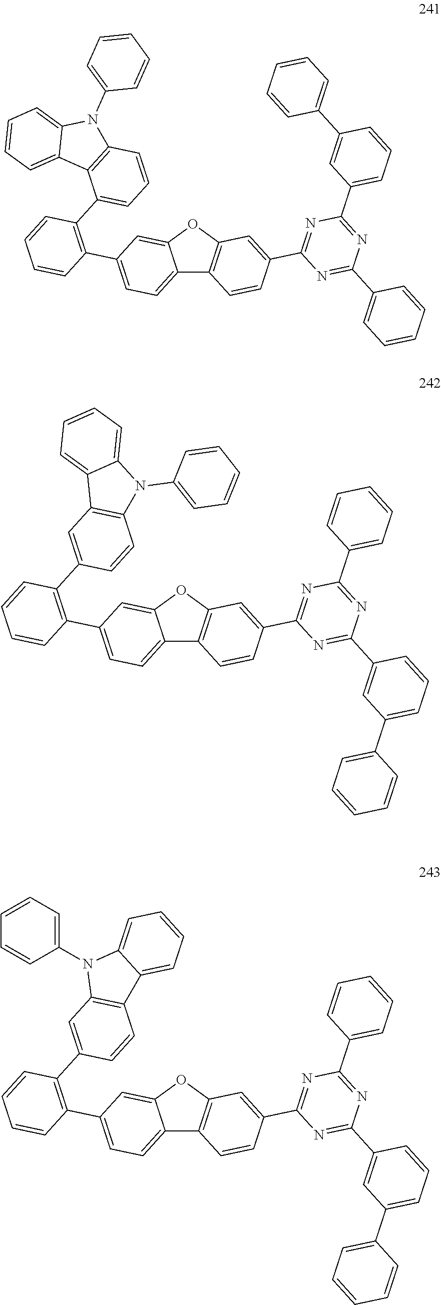 [SMILES: c1ccc(-c2cccc(-c3nc(-c4ccccc4)nc(-c4ccc5c(c4)oc4cc(-c6ccccc6-c6cccc7c6c6ccccc6n7-c6ccccc6)ccc45)n3)c2)cc1 ; c1ccc(-c2cccc(-c3nc(-c4ccccc4)nc(-c4ccc5c(c4)oc4cc(-c6ccccc6-c6ccc7c(c6)c6ccccc6n7-c6ccccc6)ccc45)n3)c2)cc1 ; c1ccc(-c2cccc(-c3nc(-c4ccccc4)nc(-c4ccc5c(c4)oc4cc(-c6ccccc6-c6ccc7c8ccccc8n(-c8ccccc8)c7c6)ccc45)n3)c2)cc1]

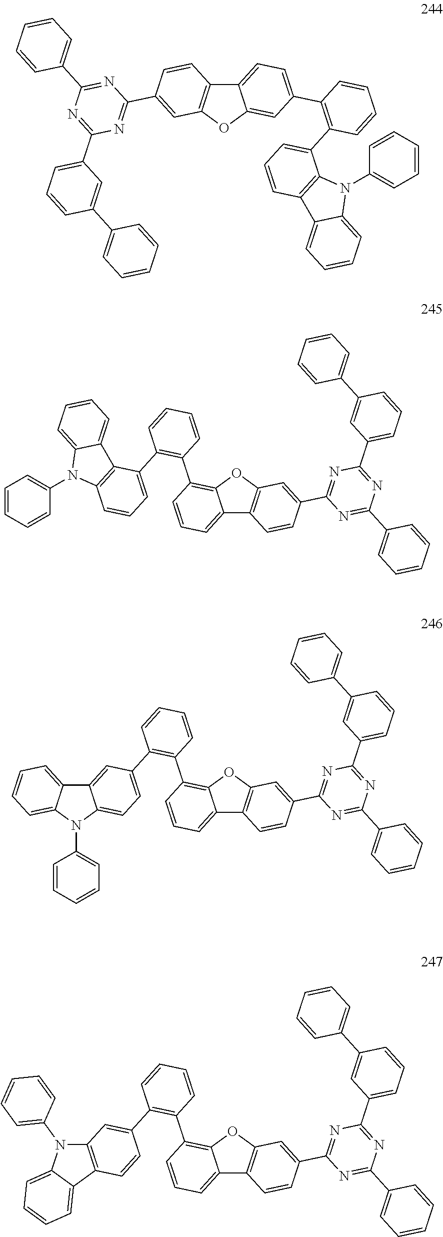 [SMILES: c1ccc(-c2cccc(-c3nc(-c4ccccc4)nc(-c4ccc5c(c4)oc4cc(-c6ccccc6-c6cccc7c8ccccc8n(-c8ccccc8)c67)ccc45)n3)c2)cc1 ; c1ccc(-c2cccc(-c3nc(-c4ccccc4)nc(-c4ccc5c(c4)oc4c(-c6ccccc6-c6cccc7c6c6ccccc6n7-c6ccccc6)cccc45)n3)c2)cc1 ; c1ccc(-c2cccc(-c3nc(-c4ccccc4)nc(-c4ccc5c(c4)oc4c(-c6ccccc6-c6ccc7c(c6)c6ccccc6n7-c6ccccc6)cccc45)n3)c2)cc1 ; c1ccc(-c2cccc(-c3nc(-c4ccccc4)nc(-c4ccc5c(c4)oc4c(-c6ccccc6-c6ccc7c8ccccc8n(-c8ccccc8)c7c6)cccc45)n3)c2)cc1]

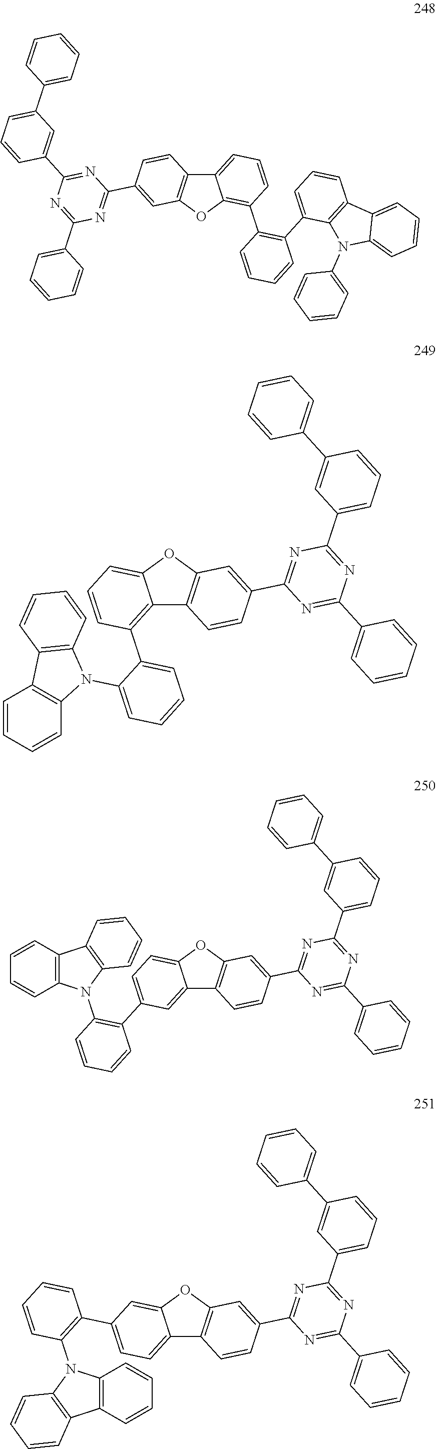 [SMILES: c1ccc(-c2cccc(-c3nc(-c4ccccc4)nc(-c4ccc5c(c4)oc4c(-c6ccccc6-c6cccc7c8ccccc8n(-c8ccccc8)c67)cccc45)n3)c2)cc1 ; c1ccc(-c2cccc(-c3nc(-c4ccccc4)nc(-c4ccc5c(c4)oc4cccc(-c6ccccc6-n6c7ccccc7c7ccccc76)c45)n3)c2)cc1 ; c1ccc(-c2cccc(-c3nc(-c4ccccc4)nc(-c4ccc5c(c4)oc4ccc(-c6ccccc6-n6c7ccccc7c7ccccc76)cc45)n3)c2)cc1 ; c1ccc(-c2cccc(-c3nc(-c4ccccc4)nc(-c4ccc5c(c4)oc4cc(-c6ccccc6-n6c7ccccc7c7ccccc76)ccc45)n3)c2)cc1]

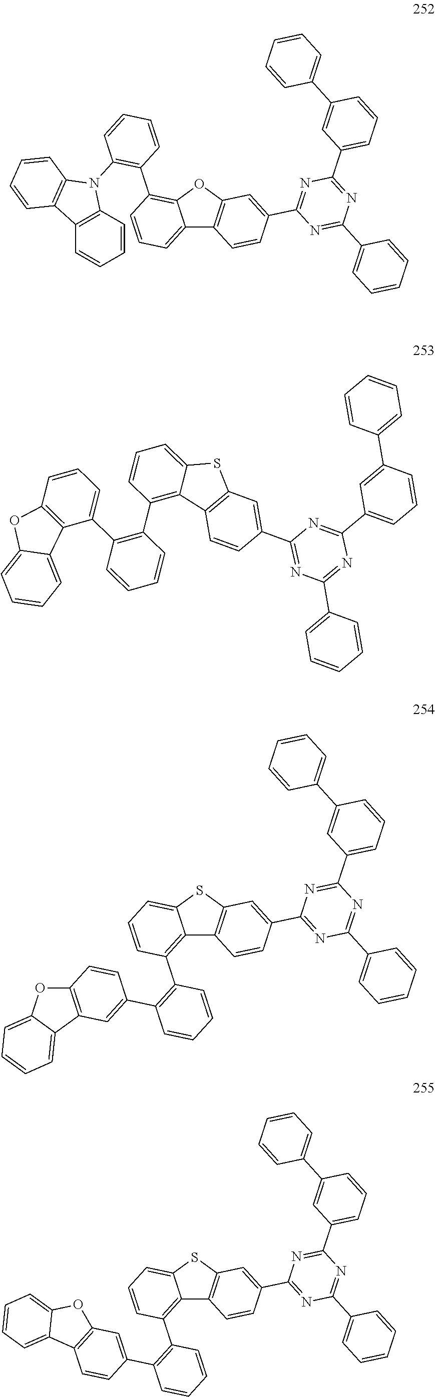 [SMILES: c1ccc(-c2cccc(-c3nc(-c4ccccc4)nc(-c4ccc5c(c4)oc4c(-c6ccccc6-n6c7ccccc7c7ccccc76)cccc45)n3)c2)cc1 ; c1ccc(-c2cccc(-c3nc(-c4ccccc4)nc(-c4ccc5c(c4)sc4cccc(-c6ccccc6-c6cccc7oc8ccccc8c67)c45)n3)c2)cc1 ; c1ccc(-c2cccc(-c3nc(-c4ccccc4)nc(-c4ccc5c(c4)sc4cccc(-c6ccccc6-c6ccc7oc8ccccc8c7c6)c45)n3)c2)cc1 ; c1ccc(-c2cccc(-c3nc(-c4ccccc4)nc(-c4ccc5c(c4)sc4cccc(-c6ccccc6-c6ccc7c(c6)oc6ccccc67)c45)n3)c2)cc1]

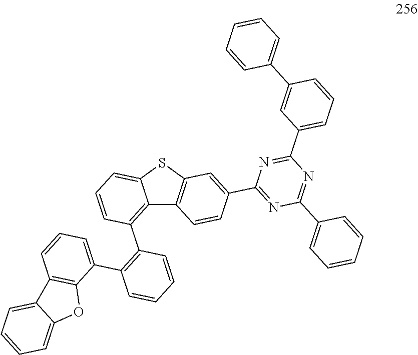 [SMILES: c1ccc(-c2cccc(-c3nc(-c4ccccc4)nc(-c4ccc5c(c4)sc4cccc(-c6ccccc6-c6cccc7c6oc6ccccc67)c45)n3)c2)cc1]

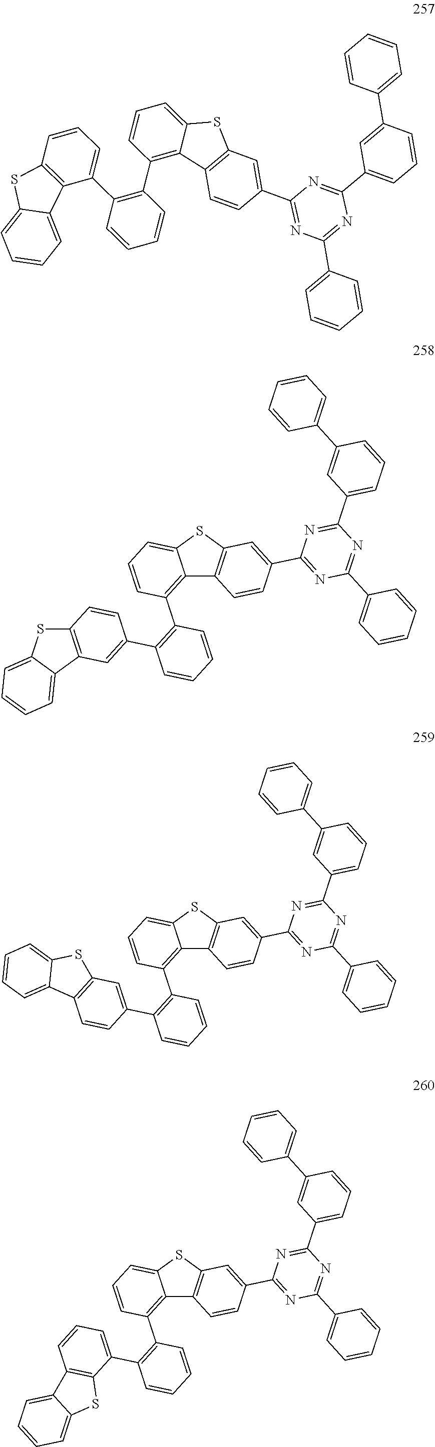 [SMILES: c1ccc(-c2cccc(-c3nc(-c4ccccc4)nc(-c4ccc5c(c4)sc4cccc(-c6ccccc6-c6cccc7sc8ccccc8c67)c45)n3)c2)cc1 ; c1ccc(-c2cccc(-c3nc(-c4ccccc4)nc(-c4ccc5c(c4)sc4cccc(-c6ccccc6-c6ccc7sc8ccccc8c7c6)c45)n3)c2)cc1 ; c1ccc(-c2cccc(-c3nc(-c4ccccc4)nc(-c4ccc5c(c4)sc4cccc(-c6ccccc6-c6ccc7c(c6)sc6ccccc67)c45)n3)c2)cc1 ; c1ccc(-c2cccc(-c3nc(-c4ccccc4)nc(-c4ccc5c(c4)sc4cccc(-c6ccccc6-c6cccc7c6sc6ccccc67)c45)n3)c2)cc1]

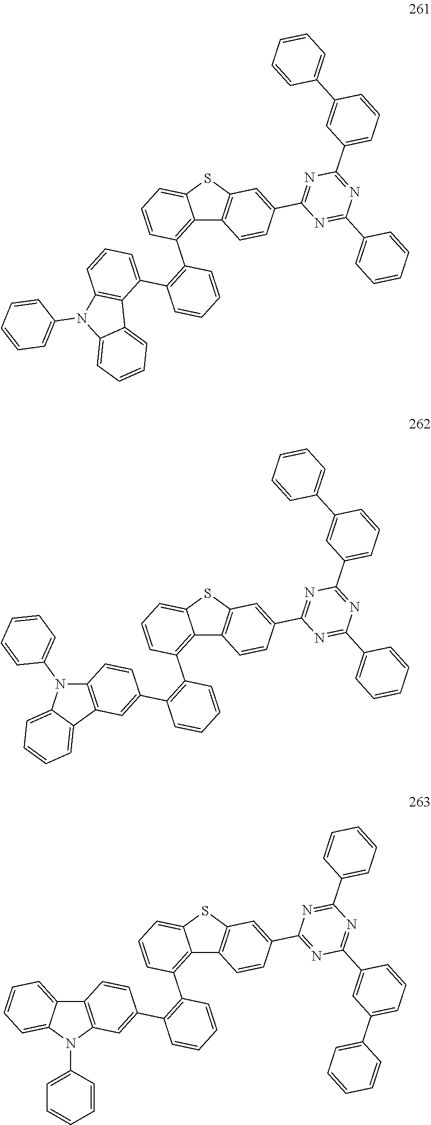 [SMILES: c1ccc(-c2cccc(-c3nc(-c4ccccc4)nc(-c4ccc5c(c4)sc4cccc(-c6ccccc6-c6cccc7c6c6ccccc6n7-c6ccccc6)c45)n3)c2)cc1 ; c1ccc(-c2cccc(-c3nc(-c4ccccc4)nc(-c4ccc5c(c4)sc4cccc(-c6ccccc6-c6ccc7c(c6)c6ccccc6n7-c6ccccc6)c45)n3)c2)cc1 ; c1ccc(-c2cccc(-c3nc(-c4ccccc4)nc(-c4ccc5c(c4)sc4cccc(-c6ccccc6-c6ccc7c8ccccc8n(-c8ccccc8)c7c6)c45)n3)c2)cc1]

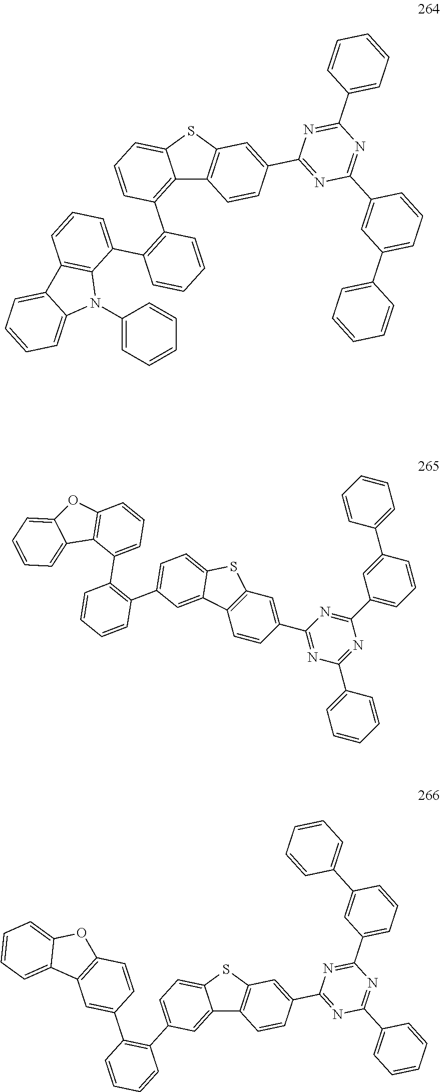 [SMILES: c1ccc(-c2cccc(-c3nc(-c4ccccc4)nc(-c4ccc5c(c4)sc4cccc(-c6ccccc6-c6cccc7c8ccccc8n(-c8ccccc8)c67)c45)n3)c2)cc1 ; c1ccc(-c2cccc(-c3nc(-c4ccccc4)nc(-c4ccc5c(c4)sc4ccc(-c6ccccc6-c6cccc7oc8ccccc8c67)cc45)n3)c2)cc1 ; c1ccc(-c2cccc(-c3nc(-c4ccccc4)nc(-c4ccc5c(c4)sc4ccc(-c6ccccc6-c6ccc7oc8ccccc8c7c6)cc45)n3)c2)cc1]

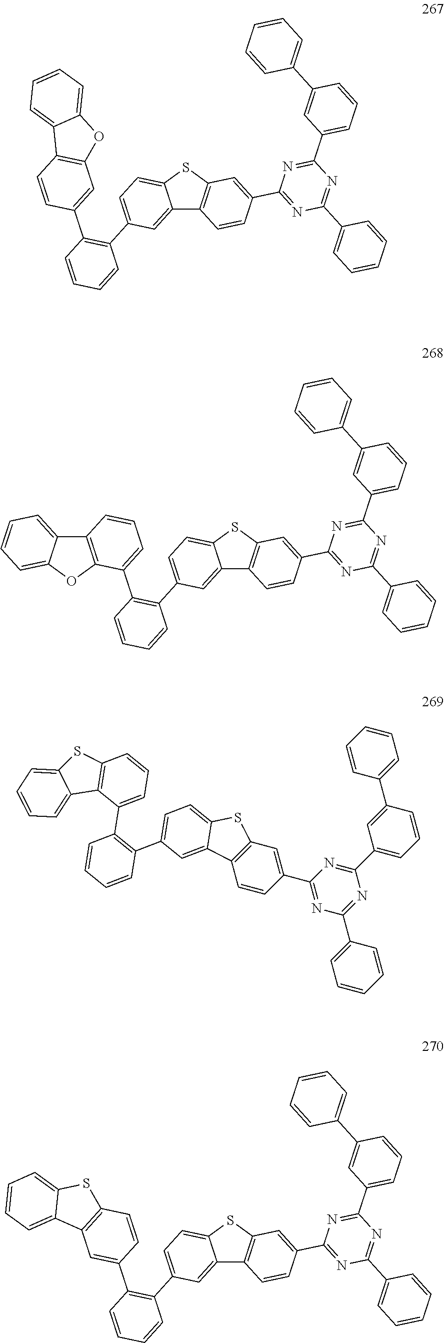 [SMILES: c1ccc(-c2cccc(-c3nc(-c4ccccc4)nc(-c4ccc5c(c4)sc4ccc(-c6ccccc6-c6ccc7c(c6)oc6ccccc67)cc45)n3)c2)cc1 ; c1ccc(-c2cccc(-c3nc(-c4ccccc4)nc(-c4ccc5c(c4)sc4ccc(-c6ccccc6-c6cccc7c6oc6ccccc67)cc45)n3)c2)cc1 ; c1ccc(-c2cccc(-c3nc(-c4ccccc4)nc(-c4ccc5c(c4)sc4ccc(-c6ccccc6-c6cccc7sc8ccccc8c67)cc45)n3)c2)cc1 ; c1ccc(-c2cccc(-c3nc(-c4ccccc4)nc(-c4ccc5c(c4)sc4ccc(-c6ccccc6-c6ccc7sc8ccccc8c7c6)cc45)n3)c2)cc1]

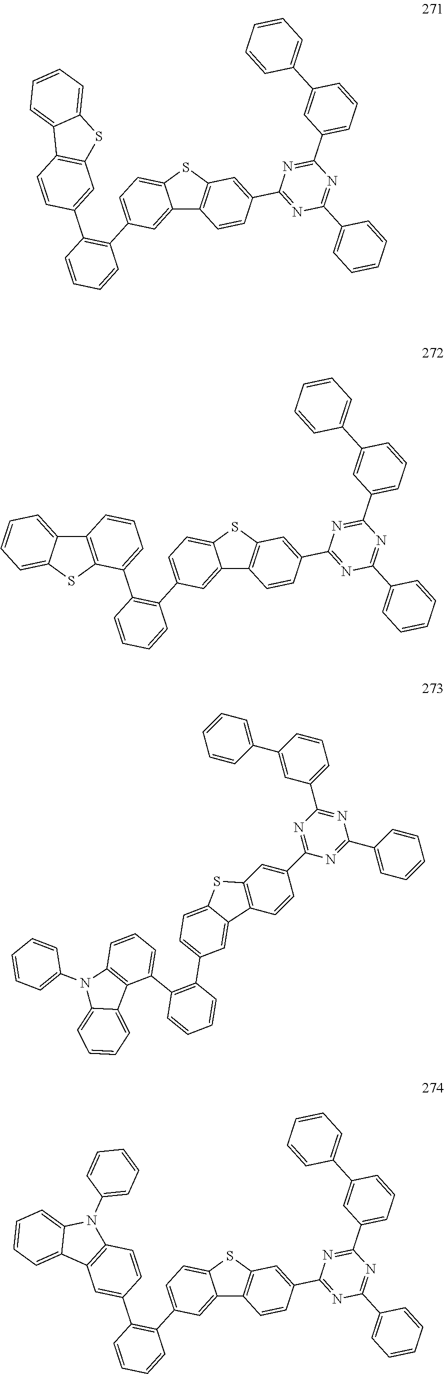 [SMILES: c1ccc(-c2cccc(-c3nc(-c4ccccc4)nc(-c4ccc5c(c4)sc4ccc(-c6ccccc6-c6ccc7c(c6)sc6ccccc67)cc45)n3)c2)cc1 ; c1ccc(-c2cccc(-c3nc(-c4ccccc4)nc(-c4ccc5c(c4)sc4ccc(-c6ccccc6-c6cccc7c6sc6ccccc67)cc45)n3)c2)cc1 ; c1ccc(-c2cccc(-c3nc(-c4ccccc4)nc(-c4ccc5c(c4)sc4ccc(-c6ccccc6-c6cccc7c6c6ccccc6n7-c6ccccc6)cc45)n3)c2)cc1 ; c1ccc(-c2cccc(-c3nc(-c4ccccc4)nc(-c4ccc5c(c4)sc4ccc(-c6ccccc6-c6ccc7c(c6)c6ccccc6n7-c6ccccc6)cc45)n3)c2)cc1]

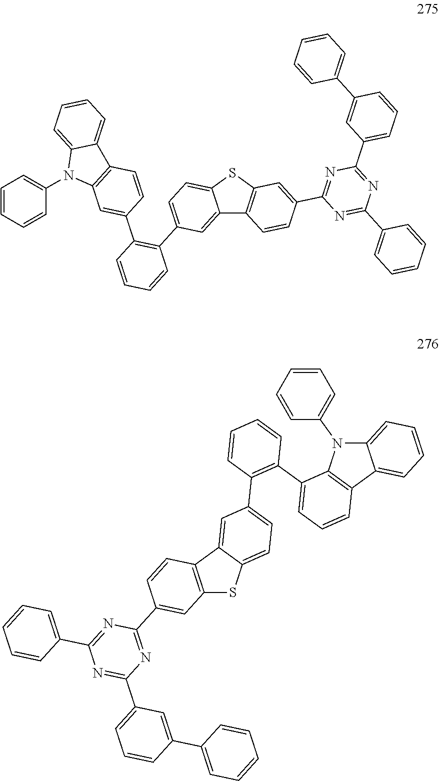 [SMILES: c1ccc(-c2cccc(-c3nc(-c4ccccc4)nc(-c4ccc5c(c4)sc4ccc(-c6ccccc6-c6ccc7c8ccccc8n(-c8ccccc8)c7c6)cc45)n3)c2)cc1 ; c1ccc(-c2cccc(-c3nc(-c4ccccc4)nc(-c4ccc5c(c4)sc4ccc(-c6ccccc6-c6cccc7c8ccccc8n(-c8ccccc8)c67)cc45)n3)c2)cc1]

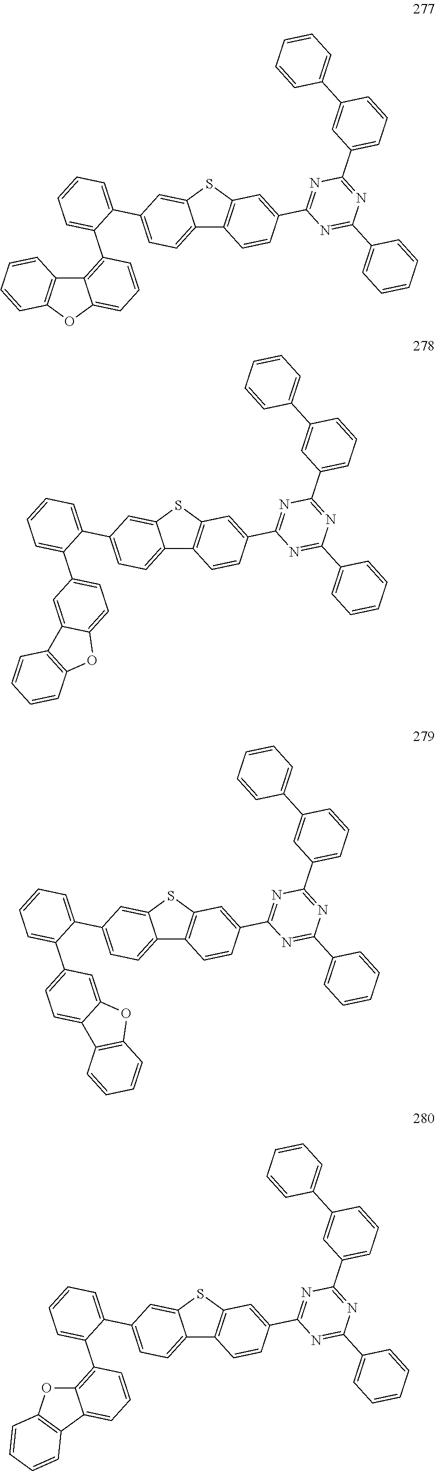 [SMILES: c1ccc(-c2cccc(-c3nc(-c4ccccc4)nc(-c4ccc5c(c4)sc4cc(-c6ccccc6-c6cccc7oc8ccccc8c67)ccc45)n3)c2)cc1 ; c1ccc(-c2cccc(-c3nc(-c4ccccc4)nc(-c4ccc5c(c4)sc4cc(-c6ccccc6-c6ccc7oc8ccccc8c7c6)ccc45)n3)c2)cc1 ; c1ccc(-c2cccc(-c3nc(-c4ccccc4)nc(-c4ccc5c(c4)sc4cc(-c6ccccc6-c6ccc7c(c6)oc6ccccc67)ccc45)n3)c2)cc1 ; c1ccc(-c2cccc(-c3nc(-c4ccccc4)nc(-c4ccc5c(c4)sc4cc(-c6ccccc6-c6cccc7c6oc6ccccc67)ccc45)n3)c2)cc1]

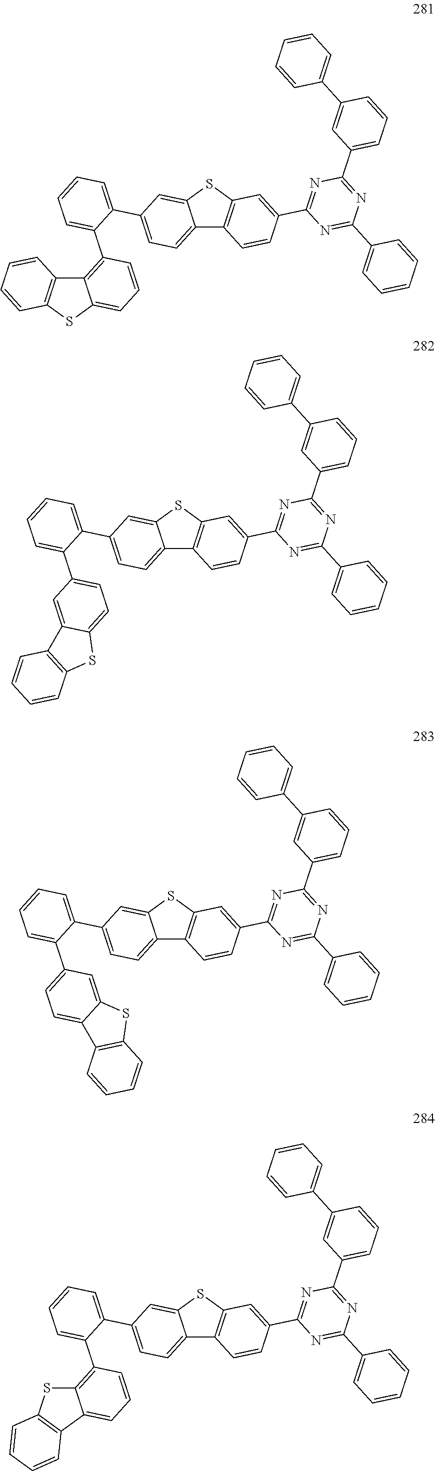 [SMILES: c1ccc(-c2cccc(-c3nc(-c4ccccc4)nc(-c4ccc5c(c4)sc4cc(-c6ccccc6-c6cccc7sc8ccccc8c67)ccc45)n3)c2)cc1 ; c1ccc(-c2cccc(-c3nc(-c4ccccc4)nc(-c4ccc5c(c4)sc4cc(-c6ccccc6-c6ccc7sc8ccccc8c7c6)ccc45)n3)c2)cc1 ; c1ccc(-c2cccc(-c3nc(-c4ccccc4)nc(-c4ccc5c(c4)sc4cc(-c6ccccc6-c6ccc7c(c6)sc6ccccc67)ccc45)n3)c2)cc1 ; c1ccc(-c2cccc(-c3nc(-c4ccccc4)nc(-c4ccc5c(c4)sc4cc(-c6ccccc6-c6cccc7c6sc6ccccc67)ccc45)n3)c2)cc1]

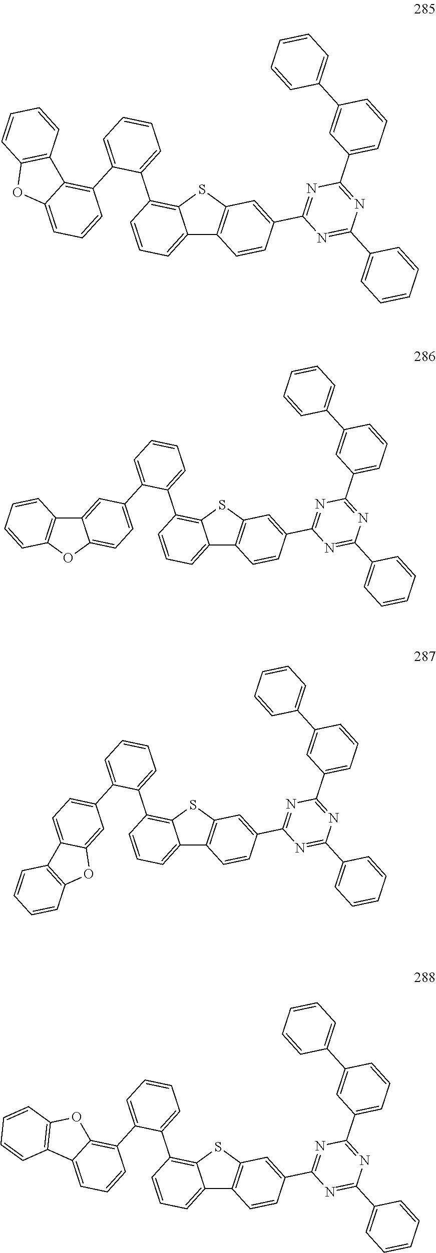 [SMILES: c1ccc(-c2cccc(-c3nc(-c4ccccc4)nc(-c4ccc5c(c4)sc4c(-c6ccccc6-c6cccc7oc8ccccc8c67)cccc45)n3)c2)cc1 ; c1ccc(-c2cccc(-c3nc(-c4ccccc4)nc(-c4ccc5c(c4)sc4c(-c6ccccc6-c6ccc7oc8ccccc8c7c6)cccc45)n3)c2)cc1 ; c1ccc(-c2cccc(-c3nc(-c4ccccc4)nc(-c4ccc5c(c4)sc4c(-c6ccccc6-c6ccc7c(c6)oc6ccccc67)cccc45)n3)c2)cc1 ; c1ccc(-c2cccc(-c3nc(-c4ccccc4)nc(-c4ccc5c(c4)sc4c(-c6ccccc6-c6cccc7c6oc6ccccc67)cccc45)n3)c2)cc1]

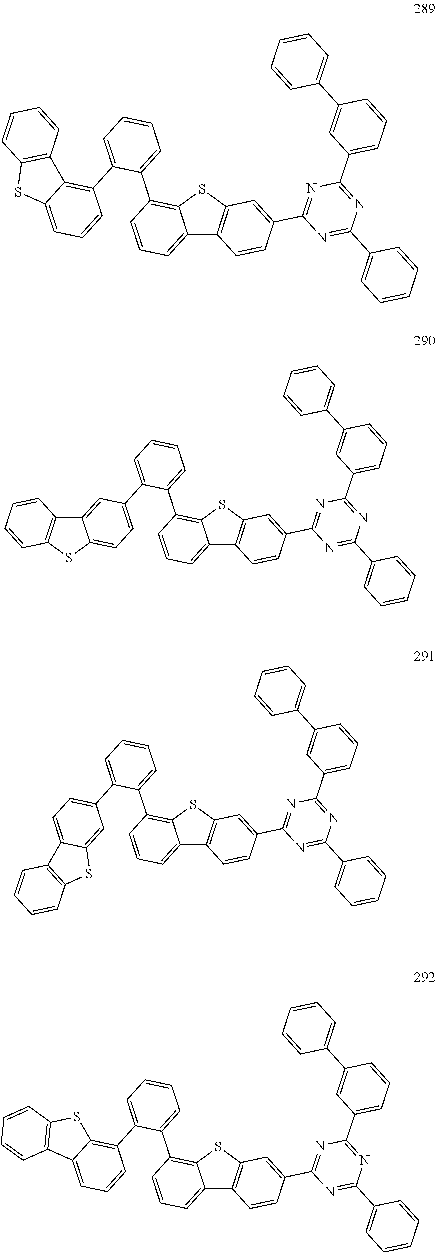 [SMILES: c1ccc(-c2cccc(-c3nc(-c4ccccc4)nc(-c4ccc5c(c4)sc4c(-c6ccccc6-c6cccc7sc8ccccc8c67)cccc45)n3)c2)cc1 ; c1ccc(-c2cccc(-c3nc(-c4ccccc4)nc(-c4ccc5c(c4)sc4c(-c6ccccc6-c6ccc7sc8ccccc8c7c6)cccc45)n3)c2)cc1 ; c1ccc(-c2cccc(-c3nc(-c4ccccc4)nc(-c4ccc5c(c4)sc4c(-c6ccccc6-c6ccc7c(c6)sc6ccccc67)cccc45)n3)c2)cc1 ; c1ccc(-c2cccc(-c3nc(-c4ccccc4)nc(-c4ccc5c(c4)sc4c(-c6ccccc6-c6cccc7c6sc6ccccc67)cccc45)n3)c2)cc1]

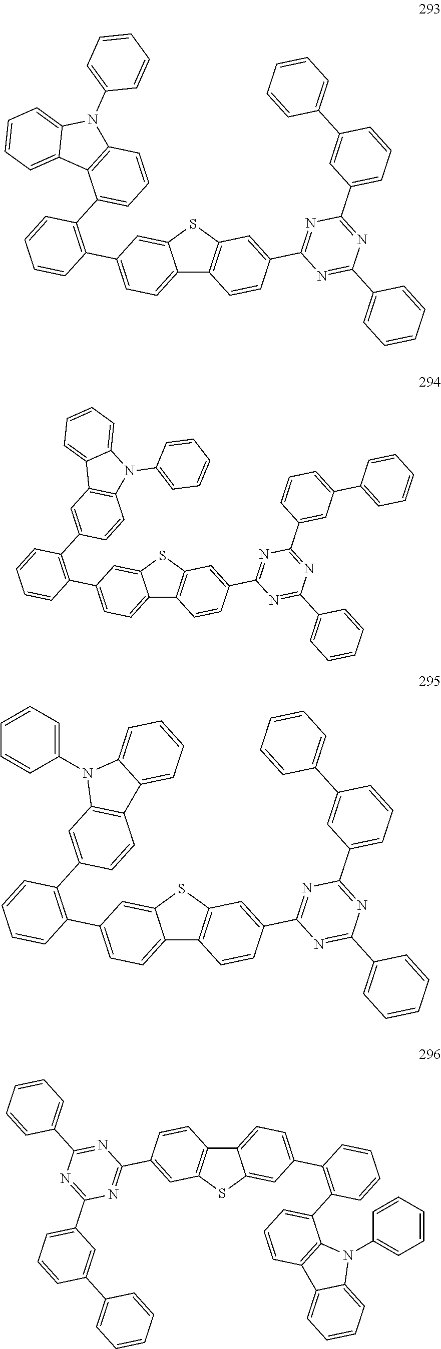 [SMILES: c1ccc(-c2cccc(-c3nc(-c4ccccc4)nc(-c4ccc5c(c4)sc4cc(-c6ccccc6-c6cccc7c6c6ccccc6n7-c6ccccc6)ccc45)n3)c2)cc1 ; c1ccc(-c2cccc(-c3nc(-c4ccccc4)nc(-c4ccc5c(c4)sc4cc(-c6ccccc6-c6ccc7c(c6)c6ccccc6n7-c6ccccc6)ccc45)n3)c2)cc1 ; c1ccc(-c2cccc(-c3nc(-c4ccccc4)nc(-c4ccc5c(c4)sc4cc(-c6ccccc6-c6ccc7c8ccccc8n(-c8ccccc8)c7c6)ccc45)n3)c2)cc1 ; c1ccc(-c2cccc(-c3nc(-c4ccccc4)nc(-c4ccc5c(c4)sc4cc(-c6ccccc6-c6cccc7c8ccccc8n(-c8ccccc8)c67)ccc45)n3)c2)cc1]

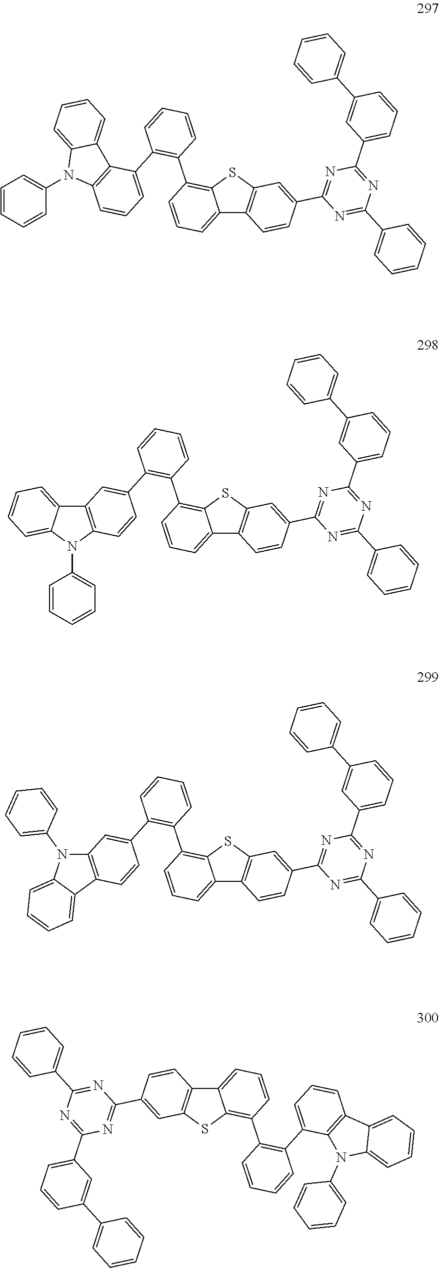 [SMILES: c1ccc(-c2cccc(-c3nc(-c4ccccc4)nc(-c4ccc5c(c4)sc4c(-c6ccccc6-c6cccc7c6c6ccccc6n7-c6ccccc6)cccc45)n3)c2)cc1 ; c1ccc(-c2cccc(-c3nc(-c4ccccc4)nc(-c4ccc5c(c4)sc4c(-c6ccccc6-c6ccc7c(c6)c6ccccc6n7-c6ccccc6)cccc45)n3)c2)cc1 ; c1ccc(-c2cccc(-c3nc(-c4ccccc4)nc(-c4ccc5c(c4)sc4c(-c6ccccc6-c6ccc7c8ccccc8n(-c8ccccc8)c7c6)cccc45)n3)c2)cc1 ; c1ccc(-c2cccc(-c3nc(-c4ccccc4)nc(-c4ccc5c(c4)sc4c(-c6ccccc6-c6cccc7c8ccccc8n(-c8ccccc8)c67)cccc45)n3)c2)cc1]

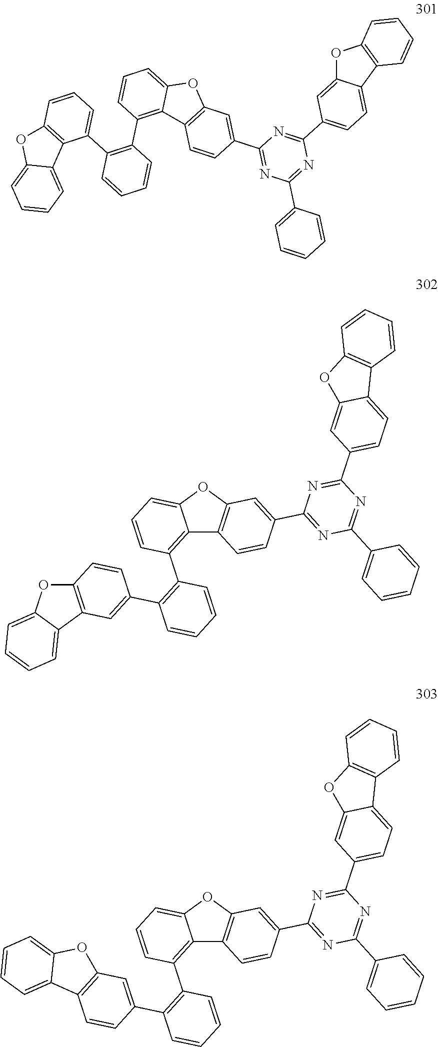 [SMILES: c1ccc(-c2nc(-c3ccc4c(c3)oc3ccccc34)nc(-c3ccc4c(c3)oc3cccc(-c5ccccc5-c5cccc6oc7ccccc7c56)c34)n2)cc1 ; c1ccc(-c2nc(-c3ccc4c(c3)oc3ccccc34)nc(-c3ccc4c(c3)oc3cccc(-c5ccccc5-c5ccc6oc7ccccc7c6c5)c34)n2)cc1 ; c1ccc(-c2nc(-c3ccc4c(c3)oc3ccccc34)nc(-c3ccc4c(c3)oc3cccc(-c5ccccc5-c5ccc6c(c5)oc5ccccc56)c34)n2)cc1]

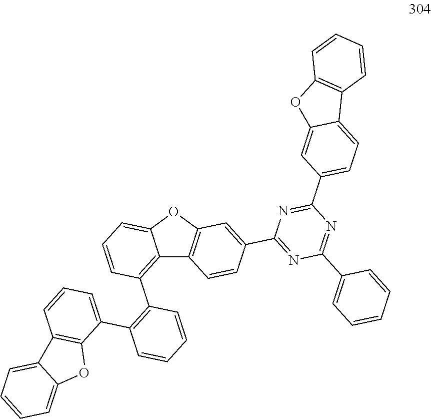 [SMILES: c1ccc(-c2nc(-c3ccc4c(c3)oc3ccccc34)nc(-c3ccc4c(c3)oc3cccc(-c5ccccc5-c5cccc6c5oc5ccccc56)c34)n2)cc1]

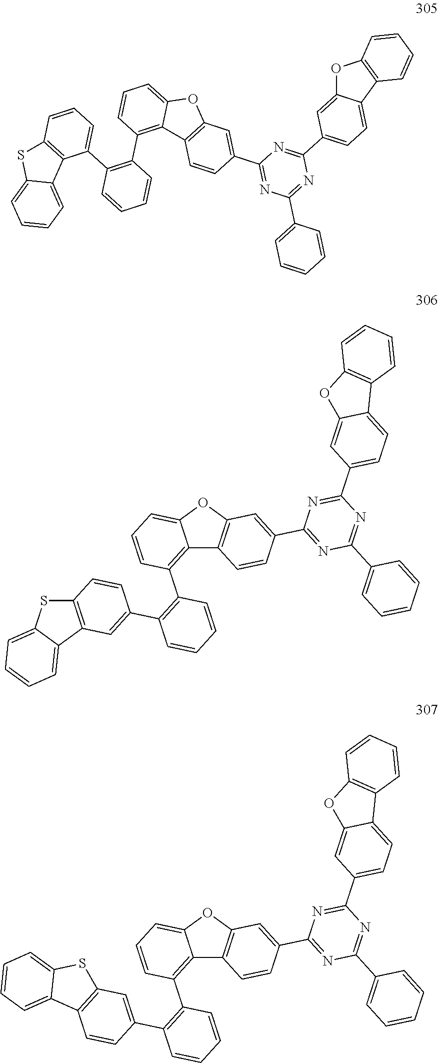 [SMILES: c1ccc(-c2nc(-c3ccc4c(c3)oc3ccccc34)nc(-c3ccc4c(c3)oc3cccc(-c5ccccc5-c5cccc6sc7ccccc7c56)c34)n2)cc1 ; c1ccc(-c2nc(-c3ccc4c(c3)oc3ccccc34)nc(-c3ccc4c(c3)oc3cccc(-c5ccccc5-c5ccc6sc7ccccc7c6c5)c34)n2)cc1 ; c1ccc(-c2nc(-c3ccc4c(c3)oc3ccccc34)nc(-c3ccc4c(c3)oc3cccc(-c5ccccc5-c5ccc6c(c5)sc5ccccc56)c34)n2)cc1]

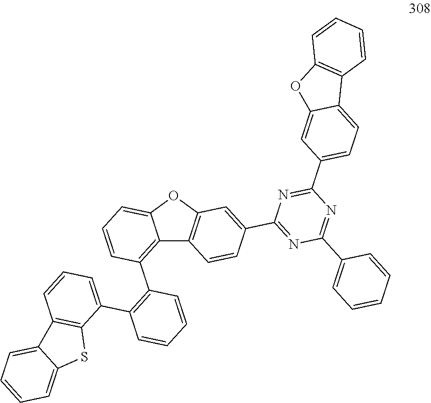 [SMILES: c1ccc(-c2nc(-c3ccc4c(c3)oc3ccccc34)nc(-c3ccc4c(c3)oc3cccc(-c5ccccc5-c5cccc6c5sc5ccccc56)c34)n2)cc1]

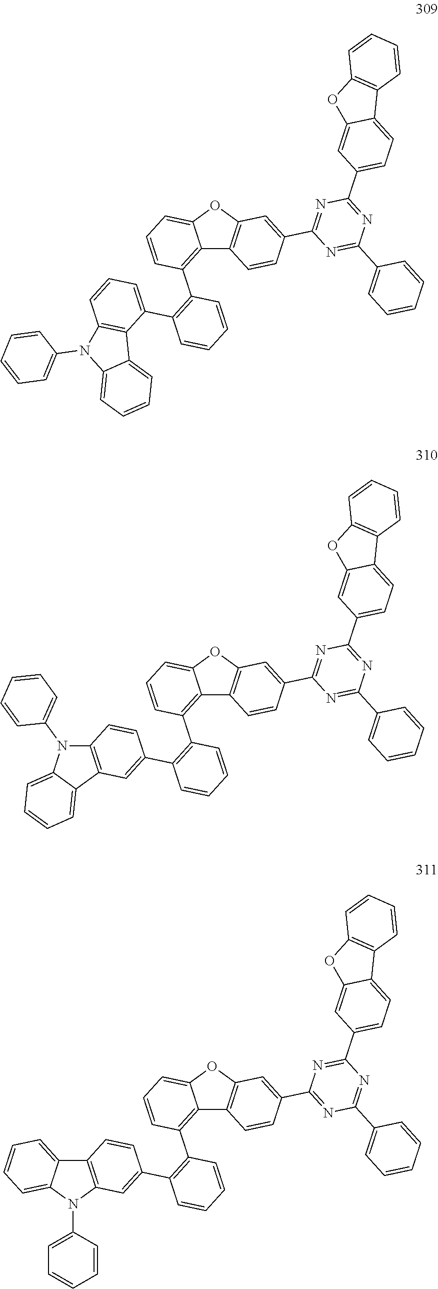 [SMILES: c1ccc(-c2nc(-c3ccc4c(c3)oc3ccccc34)nc(-c3ccc4c(c3)oc3cccc(-c5ccccc5-c5cccc6c5c5ccccc5n6-c5ccccc5)c34)n2)cc1 ; c1ccc(-c2nc(-c3ccc4c(c3)oc3ccccc34)nc(-c3ccc4c(c3)oc3cccc(-c5ccccc5-c5ccc6c(c5)c5ccccc5n6-c5ccccc5)c34)n2)cc1 ; c1ccc(-c2nc(-c3ccc4c(c3)oc3ccccc34)nc(-c3ccc4c(c3)oc3cccc(-c5ccccc5-c5ccc6c7ccccc7n(-c7ccccc7)c6c5)c34)n2)cc1]

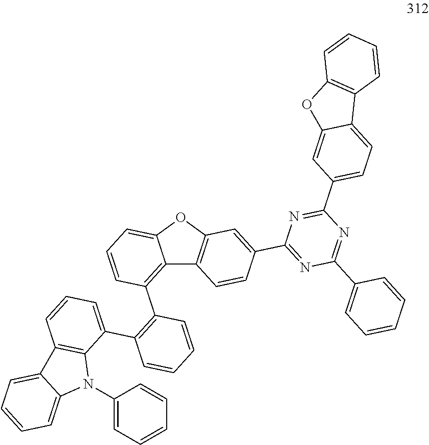 [SMILES: c1ccc(-c2nc(-c3ccc4c(c3)oc3ccccc34)nc(-c3ccc4c(c3)oc3cccc(-c5ccccc5-c5cccc6c7ccccc7n(-c7ccccc7)c56)c34)n2)cc1]

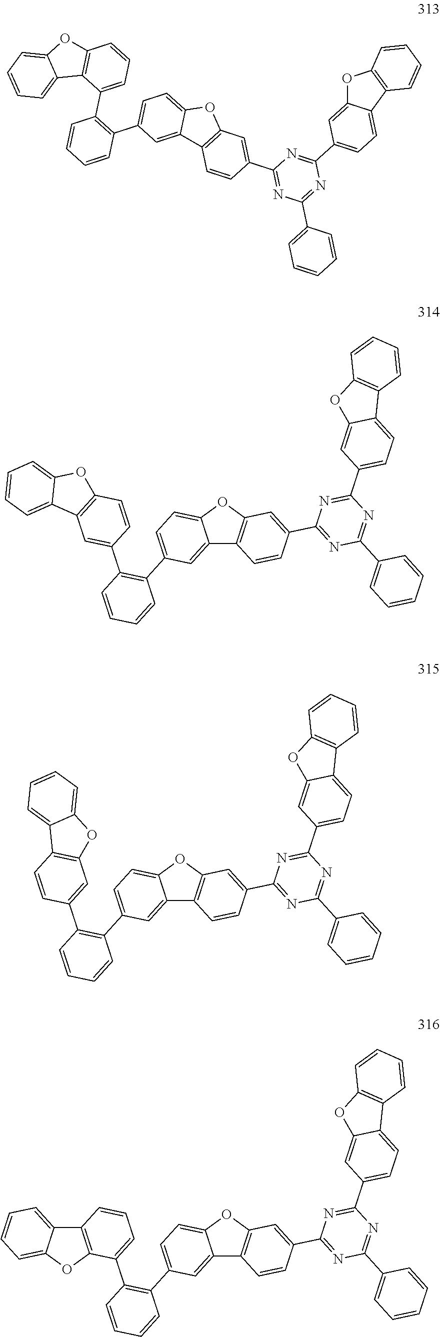 [SMILES: c1ccc(-c2nc(-c3ccc4c(c3)oc3ccccc34)nc(-c3ccc4c(c3)oc3ccc(-c5ccccc5-c5cccc6oc7ccccc7c56)cc34)n2)cc1 ; c1ccc(-c2nc(-c3ccc4c(c3)oc3ccccc34)nc(-c3ccc4c(c3)oc3ccc(-c5ccccc5-c5ccc6oc7ccccc7c6c5)cc34)n2)cc1 ; c1ccc(-c2nc(-c3ccc4c(c3)oc3ccccc34)nc(-c3ccc4c(c3)oc3ccc(-c5ccccc5-c5ccc6c(c5)oc5ccccc56)cc34)n2)cc1 ; c1ccc(-c2nc(-c3ccc4c(c3)oc3ccccc34)nc(-c3ccc4c(c3)oc3ccc(-c5ccccc5-c5cccc6c5oc5ccccc56)cc34)n2)cc1]

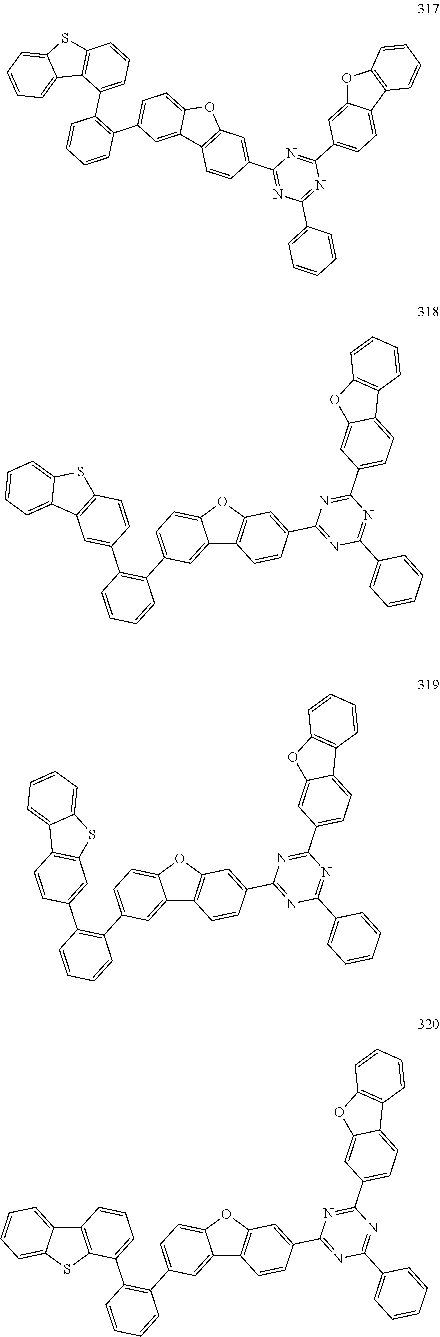 [SMILES: c1ccc(-c2nc(-c3ccc4c(c3)oc3ccccc34)nc(-c3ccc4c(c3)oc3ccc(-c5ccccc5-c5cccc6sc7ccccc7c56)cc34)n2)cc1 ; c1ccc(-c2nc(-c3ccc4c(c3)oc3ccccc34)nc(-c3ccc4c(c3)oc3ccc(-c5ccccc5-c5ccc6sc7ccccc7c6c5)cc34)n2)cc1 ; c1ccc(-c2nc(-c3ccc4c(c3)oc3ccccc34)nc(-c3ccc4c(c3)oc3ccc(-c5ccccc5-c5ccc6c(c5)sc5ccccc56)cc34)n2)cc1 ; c1ccc(-c2nc(-c3ccc4c(c3)oc3ccccc34)nc(-c3ccc4c(c3)oc3ccc(-c5ccccc5-c5cccc6c5sc5ccccc56)cc34)n2)cc1]

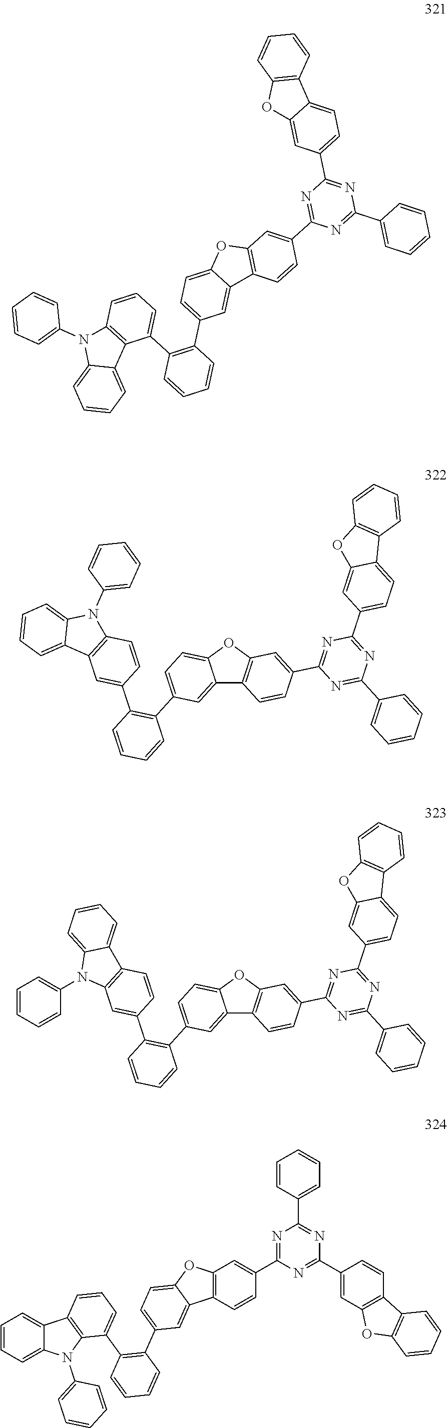 [SMILES: c1ccc(-c2nc(-c3ccc4c(c3)oc3ccccc34)nc(-c3ccc4c(c3)oc3ccc(-c5ccccc5-c5cccc6c5c5ccccc5n6-c5ccccc5)cc34)n2)cc1 ; c1ccc(-c2nc(-c3ccc4c(c3)oc3ccccc34)nc(-c3ccc4c(c3)oc3ccc(-c5ccccc5-c5ccc6c(c5)c5ccccc5n6-c5ccccc5)cc34)n2)cc1 ; c1ccc(-c2nc(-c3ccc4c(c3)oc3ccccc34)nc(-c3ccc4c(c3)oc3ccc(-c5ccccc5-c5ccc6c7ccccc7n(-c7ccccc7)c6c5)cc34)n2)cc1 ; c1ccc(-c2nc(-c3ccc4c(c3)oc3ccccc34)nc(-c3ccc4c(c3)oc3ccc(-c5ccccc5-c5cccc6c7ccccc7n(-c7ccccc7)c56)cc34)n2)cc1]

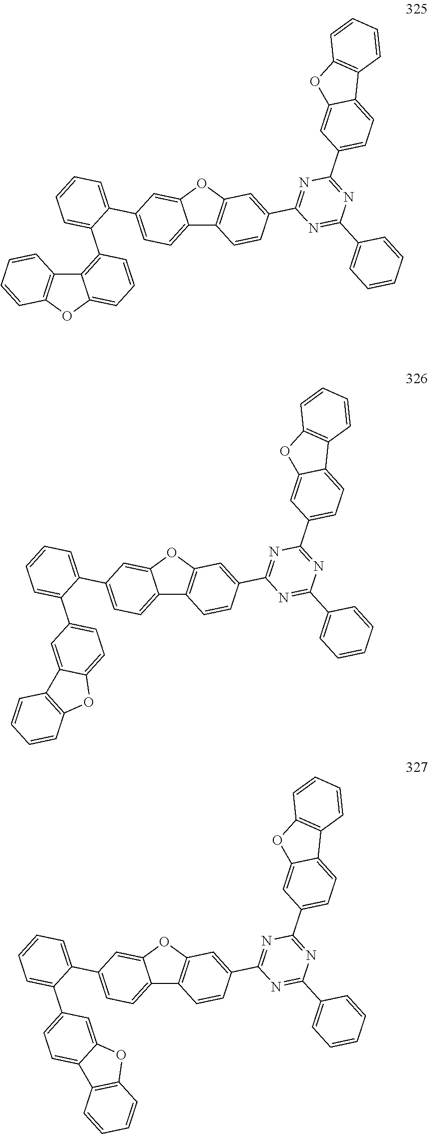 [SMILES: c1ccc(-c2nc(-c3ccc4c(c3)oc3ccccc34)nc(-c3ccc4c(c3)oc3cc(-c5ccccc5-c5cccc6oc7ccccc7c56)ccc34)n2)cc1 ; c1ccc(-c2nc(-c3ccc4c(c3)oc3ccccc34)nc(-c3ccc4c(c3)oc3cc(-c5ccccc5-c5ccc6oc7ccccc7c6c5)ccc34)n2)cc1 ; c1ccc(-c2nc(-c3ccc4c(c3)oc3ccccc34)nc(-c3ccc4c(c3)oc3cc(-c5ccccc5-c5ccc6c(c5)oc5ccccc56)ccc34)n2)cc1]

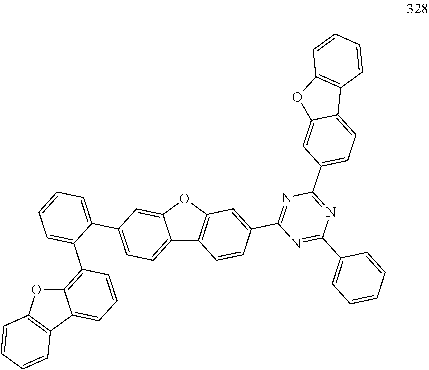 [SMILES: c1ccc(-c2nc(-c3ccc4c(c3)oc3ccccc34)nc(-c3ccc4c(c3)oc3cc(-c5ccccc5-c5cccc6c5oc5ccccc56)ccc34)n2)cc1]

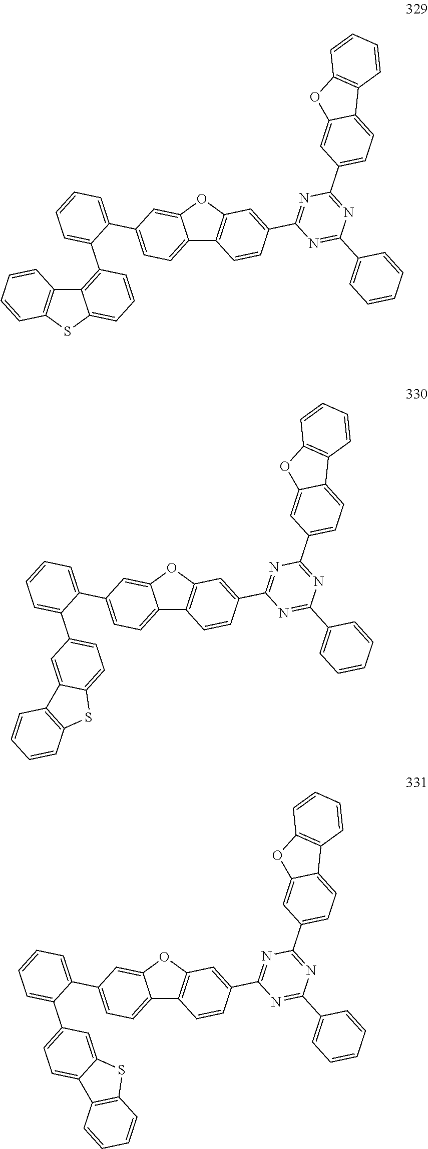 [SMILES: c1ccc(-c2nc(-c3ccc4c(c3)oc3ccccc34)nc(-c3ccc4c(c3)oc3cc(-c5ccccc5-c5cccc6sc7ccccc7c56)ccc34)n2)cc1 ; c1ccc(-c2nc(-c3ccc4c(c3)oc3ccccc34)nc(-c3ccc4c(c3)oc3cc(-c5ccccc5-c5ccc6sc7ccccc7c6c5)ccc34)n2)cc1 ; c1ccc(-c2nc(-c3ccc4c(c3)oc3ccccc34)nc(-c3ccc4c(c3)oc3cc(-c5ccccc5-c5ccc6c(c5)sc5ccccc56)ccc34)n2)cc1]

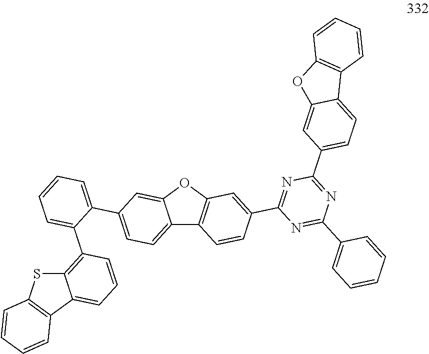 [SMILES: c1ccc(-c2nc(-c3ccc4c(c3)oc3ccccc34)nc(-c3ccc4c(c3)oc3cc(-c5ccccc5-c5cccc6c5sc5ccccc56)ccc34)n2)cc1]

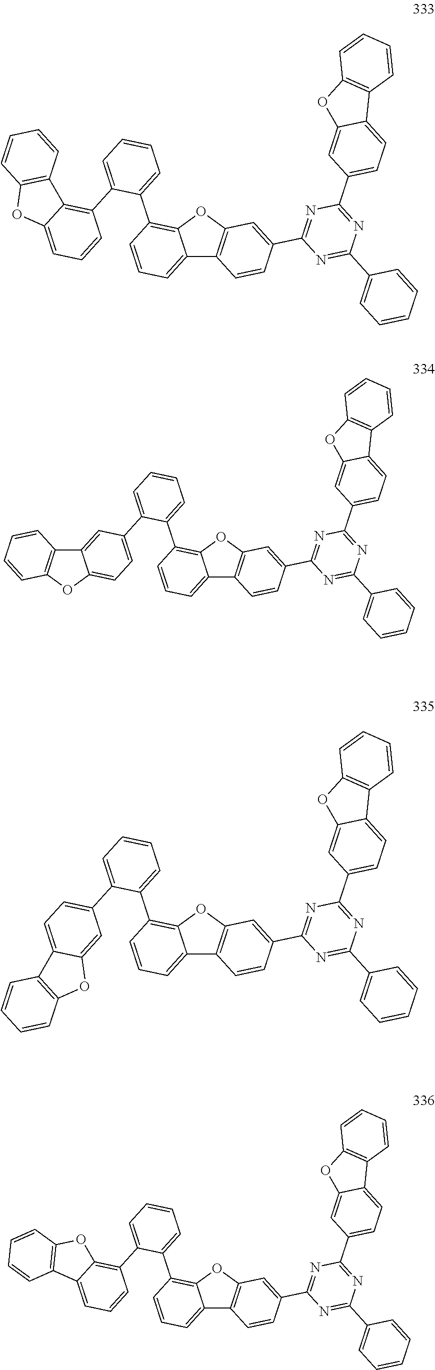 [SMILES: c1ccc(-c2nc(-c3ccc4c(c3)oc3ccccc34)nc(-c3ccc4c(c3)oc3c(-c5ccccc5-c5cccc6oc7ccccc7c56)cccc34)n2)cc1 ; c1ccc(-c2nc(-c3ccc4c(c3)oc3ccccc34)nc(-c3ccc4c(c3)oc3c(-c5ccccc5-c5ccc6oc7ccccc7c6c5)cccc34)n2)cc1 ; c1ccc(-c2nc(-c3ccc4c(c3)oc3ccccc34)nc(-c3ccc4c(c3)oc3c(-c5ccccc5-c5ccc6c(c5)oc5ccccc56)cccc34)n2)cc1 ; c1ccc(-c2nc(-c3ccc4c(c3)oc3ccccc34)nc(-c3ccc4c(c3)oc3c(-c5ccccc5-c5cccc6c5oc5ccccc56)cccc34)n2)cc1]

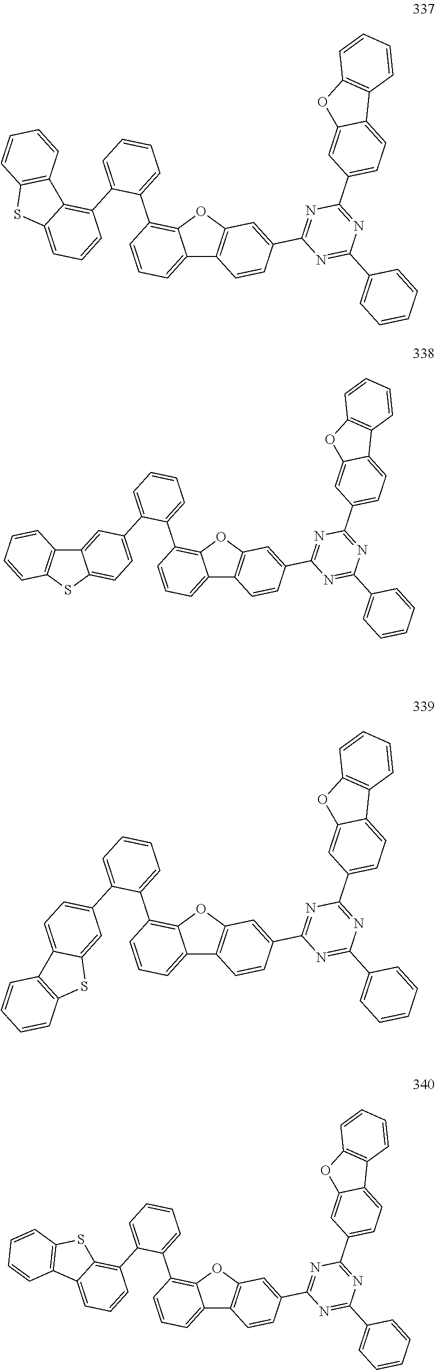 [SMILES: c1ccc(-c2nc(-c3ccc4c(c3)oc3ccccc34)nc(-c3ccc4c(c3)oc3c(-c5ccccc5-c5cccc6sc7ccccc7c56)cccc34)n2)cc1 ; c1ccc(-c2nc(-c3ccc4c(c3)oc3ccccc34)nc(-c3ccc4c(c3)oc3c(-c5ccccc5-c5ccc6sc7ccccc7c6c5)cccc34)n2)cc1 ; c1ccc(-c2nc(-c3ccc4c(c3)oc3ccccc34)nc(-c3ccc4c(c3)oc3c(-c5ccccc5-c5ccc6c(c5)sc5ccccc56)cccc34)n2)cc1 ; c1ccc(-c2nc(-c3ccc4c(c3)oc3ccccc34)nc(-c3ccc4c(c3)oc3c(-c5ccccc5-c5cccc6c5sc5ccccc56)cccc34)n2)cc1]

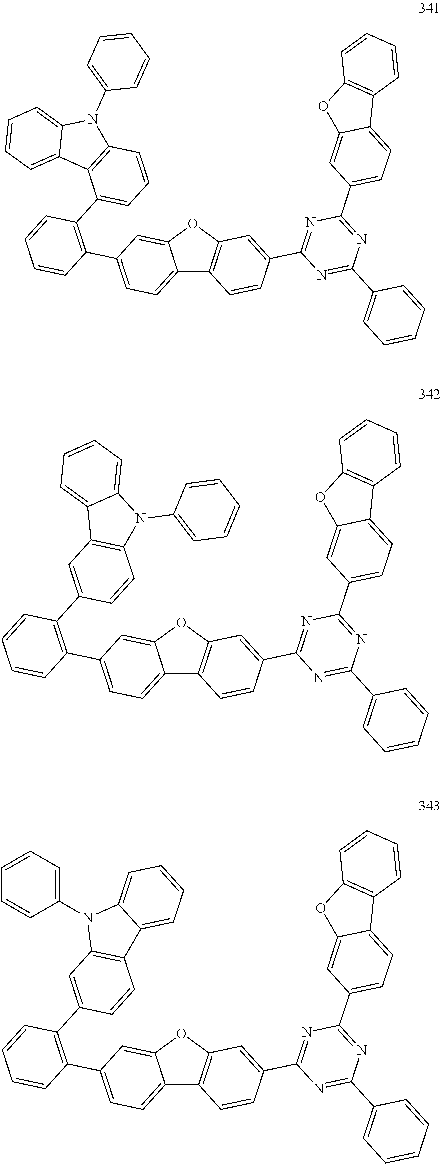 [SMILES: c1ccc(-c2nc(-c3ccc4c(c3)oc3ccccc34)nc(-c3ccc4c(c3)oc3cc(-c5ccccc5-c5cccc6c5c5ccccc5n6-c5ccccc5)ccc34)n2)cc1 ; c1ccc(-c2nc(-c3ccc4c(c3)oc3ccccc34)nc(-c3ccc4c(c3)oc3cc(-c5ccccc5-c5ccc6c(c5)c5ccccc5n6-c5ccccc5)ccc34)n2)cc1 ; c1ccc(-c2nc(-c3ccc4c(c3)oc3ccccc34)nc(-c3ccc4c(c3)oc3cc(-c5ccccc5-c5ccc6c7ccccc7n(-c7ccccc7)c6c5)ccc34)n2)cc1]

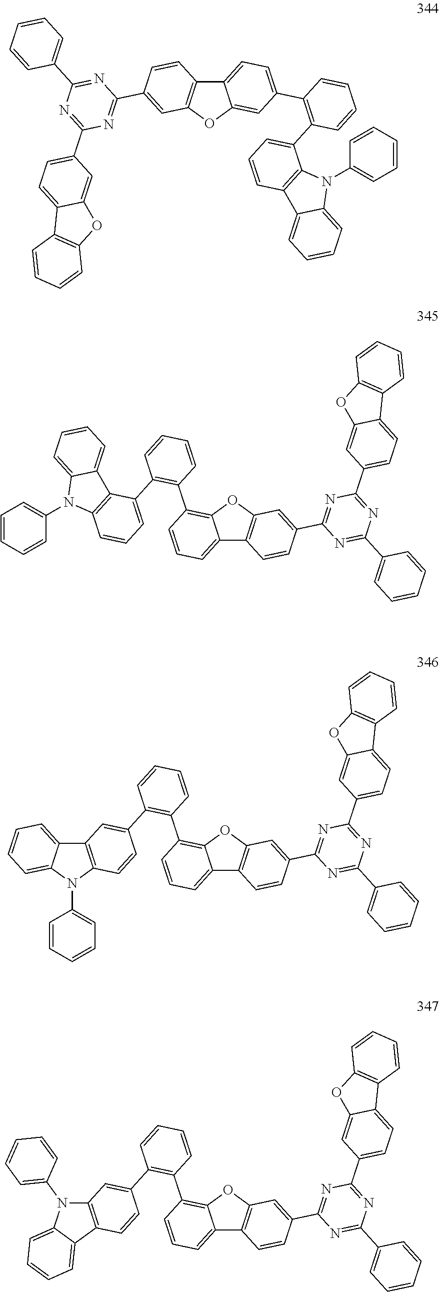 [SMILES: c1ccc(-c2nc(-c3ccc4c(c3)oc3ccccc34)nc(-c3ccc4c(c3)oc3cc(-c5ccccc5-c5cccc6c7ccccc7n(-c7ccccc7)c56)ccc34)n2)cc1 ; c1ccc(-c2nc(-c3ccc4c(c3)oc3ccccc34)nc(-c3ccc4c(c3)oc3c(-c5ccccc5-c5cccc6c5c5ccccc5n6-c5ccccc5)cccc34)n2)cc1 ; c1ccc(-c2nc(-c3ccc4c(c3)oc3ccccc34)nc(-c3ccc4c(c3)oc3c(-c5ccccc5-c5ccc6c(c5)c5ccccc5n6-c5ccccc5)cccc34)n2)cc1 ; c1ccc(-c2nc(-c3ccc4c(c3)oc3ccccc34)nc(-c3ccc4c(c3)oc3c(-c5ccccc5-c5ccc6c7ccccc7n(-c7ccccc7)c6c5)cccc34)n2)cc1]

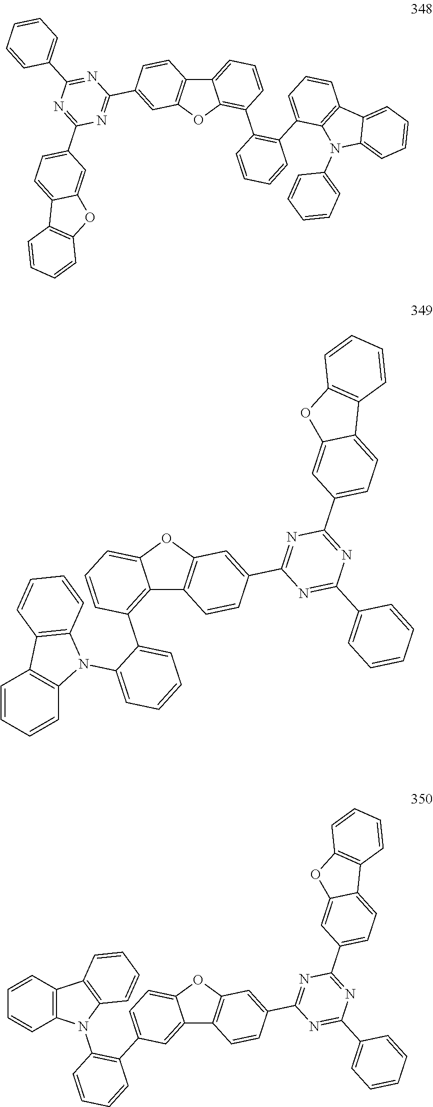 [SMILES: c1ccc(-c2nc(-c3ccc4c(c3)oc3ccccc34)nc(-c3ccc4c(c3)oc3c(-c5ccccc5-c5cccc6c7ccccc7n(-c7ccccc7)c56)cccc34)n2)cc1 ; c1ccc(-c2nc(-c3ccc4c(c3)oc3ccccc34)nc(-c3ccc4c(c3)oc3cccc(-c5ccccc5-n5c6ccccc6c6ccccc65)c34)n2)cc1 ; c1ccc(-c2nc(-c3ccc4c(c3)oc3ccccc34)nc(-c3ccc4c(c3)oc3ccc(-c5ccccc5-n5c6ccccc6c6ccccc65)cc34)n2)cc1]

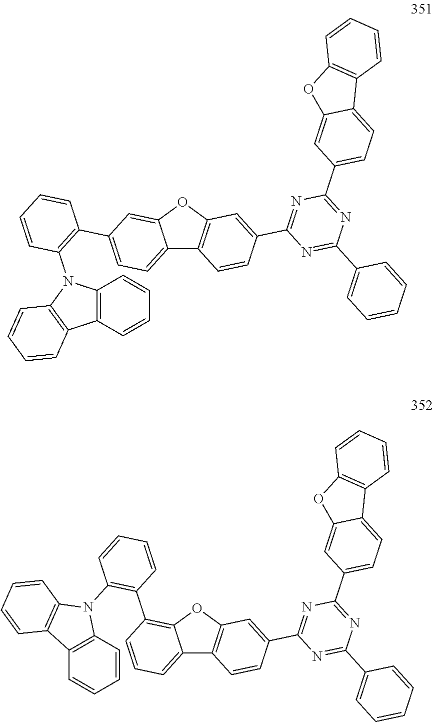 [SMILES: c1ccc(-c2nc(-c3ccc4c(c3)oc3ccccc34)nc(-c3ccc4c(c3)oc3cc(-c5ccccc5-n5c6ccccc6c6ccccc65)ccc34)n2)cc1 ; c1ccc(-c2nc(-c3ccc4c(c3)oc3ccccc34)nc(-c3ccc4c(c3)oc3c(-c5ccccc5-n5c6ccccc6c6ccccc65)cccc34)n2)cc1]

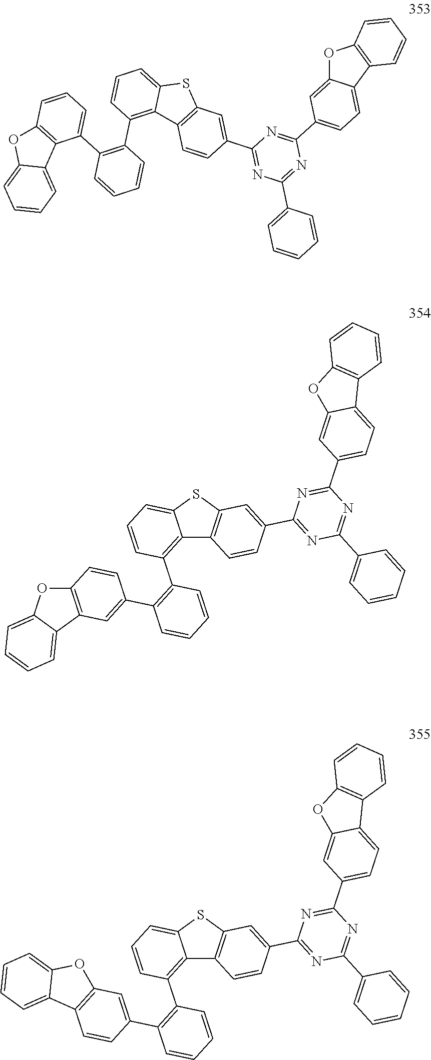 [SMILES: c1ccc(-c2nc(-c3ccc4c(c3)oc3ccccc34)nc(-c3ccc4c(c3)sc3cccc(-c5ccccc5-c5cccc6oc7ccccc7c56)c34)n2)cc1 ; c1ccc(-c2nc(-c3ccc4c(c3)oc3ccccc34)nc(-c3ccc4c(c3)sc3cccc(-c5ccccc5-c5ccc6oc7ccccc7c6c5)c34)n2)cc1 ; c1ccc(-c2nc(-c3ccc4c(c3)oc3ccccc34)nc(-c3ccc4c(c3)sc3cccc(-c5ccccc5-c5ccc6c(c5)oc5ccccc56)c34)n2)cc1]

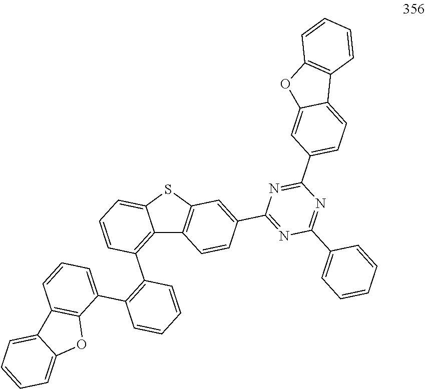 [SMILES: c1ccc(-c2nc(-c3ccc4c(c3)oc3ccccc34)nc(-c3ccc4c(c3)sc3cccc(-c5ccccc5-c5cccc6c5oc5ccccc56)c34)n2)cc1]

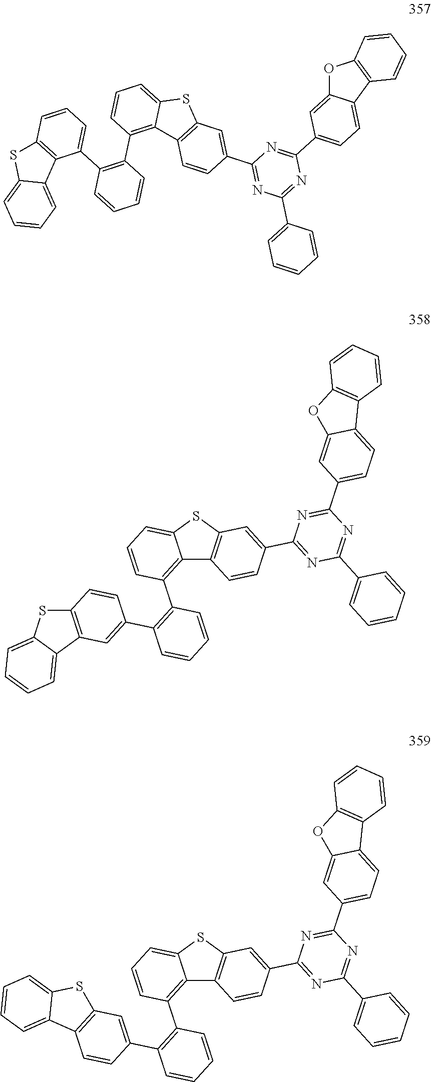 [SMILES: c1ccc(-c2nc(-c3ccc4c(c3)oc3ccccc34)nc(-c3ccc4c(c3)sc3cccc(-c5ccccc5-c5cccc6sc7ccccc7c56)c34)n2)cc1 ; c1ccc(-c2nc(-c3ccc4c(c3)oc3ccccc34)nc(-c3ccc4c(c3)sc3cccc(-c5ccccc5-c5ccc6sc7ccccc7c6c5)c34)n2)cc1 ; c1ccc(-c2nc(-c3ccc4c(c3)oc3ccccc34)nc(-c3ccc4c(c3)sc3cccc(-c5ccccc5-c5ccc6c(c5)sc5ccccc56)c34)n2)cc1]

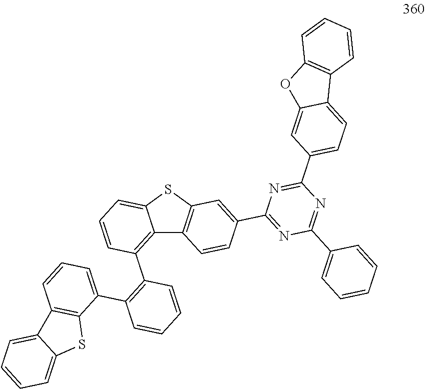 [SMILES: c1ccc(-c2nc(-c3ccc4c(c3)oc3ccccc34)nc(-c3ccc4c(c3)sc3cccc(-c5ccccc5-c5cccc6c5sc5ccccc56)c34)n2)cc1]

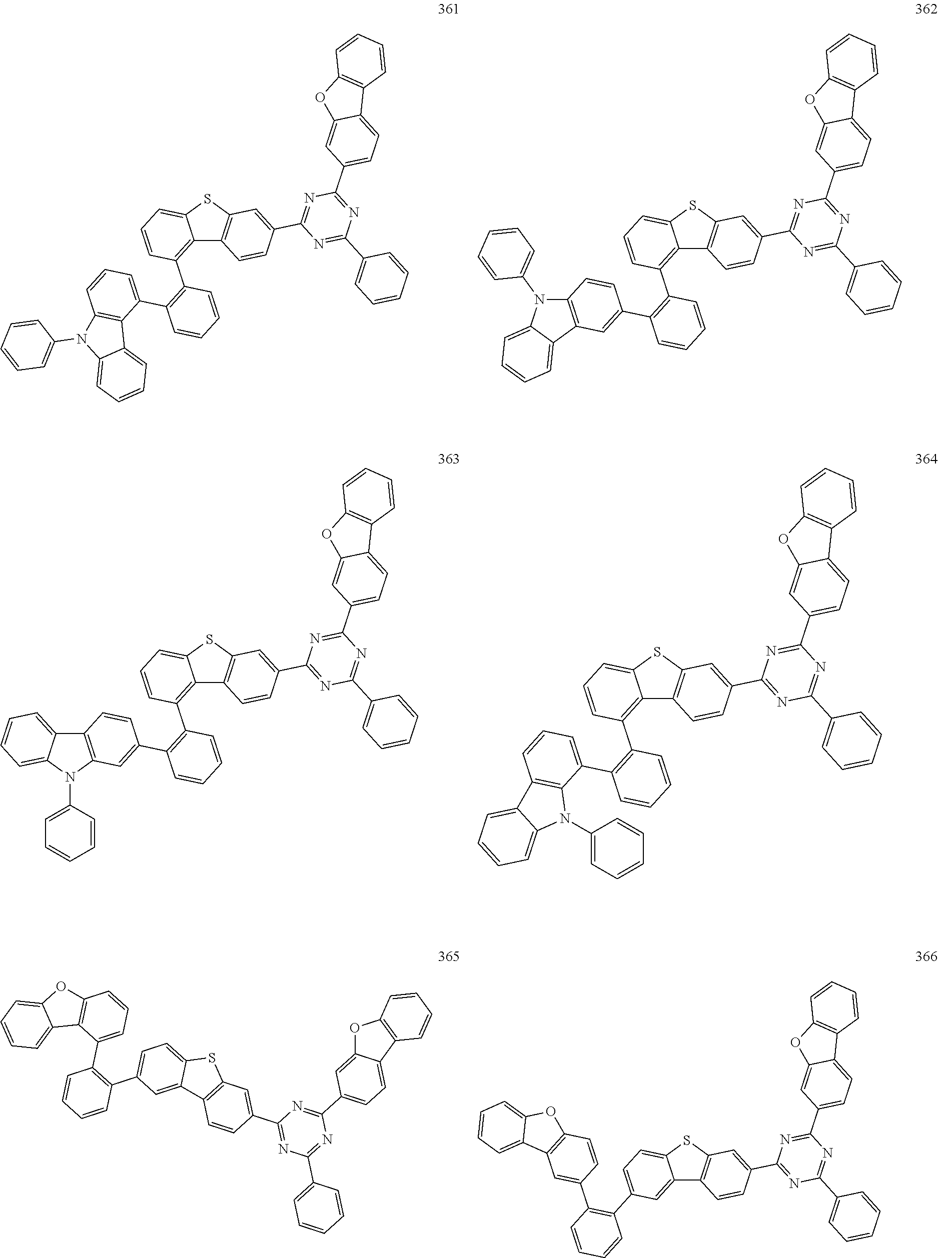

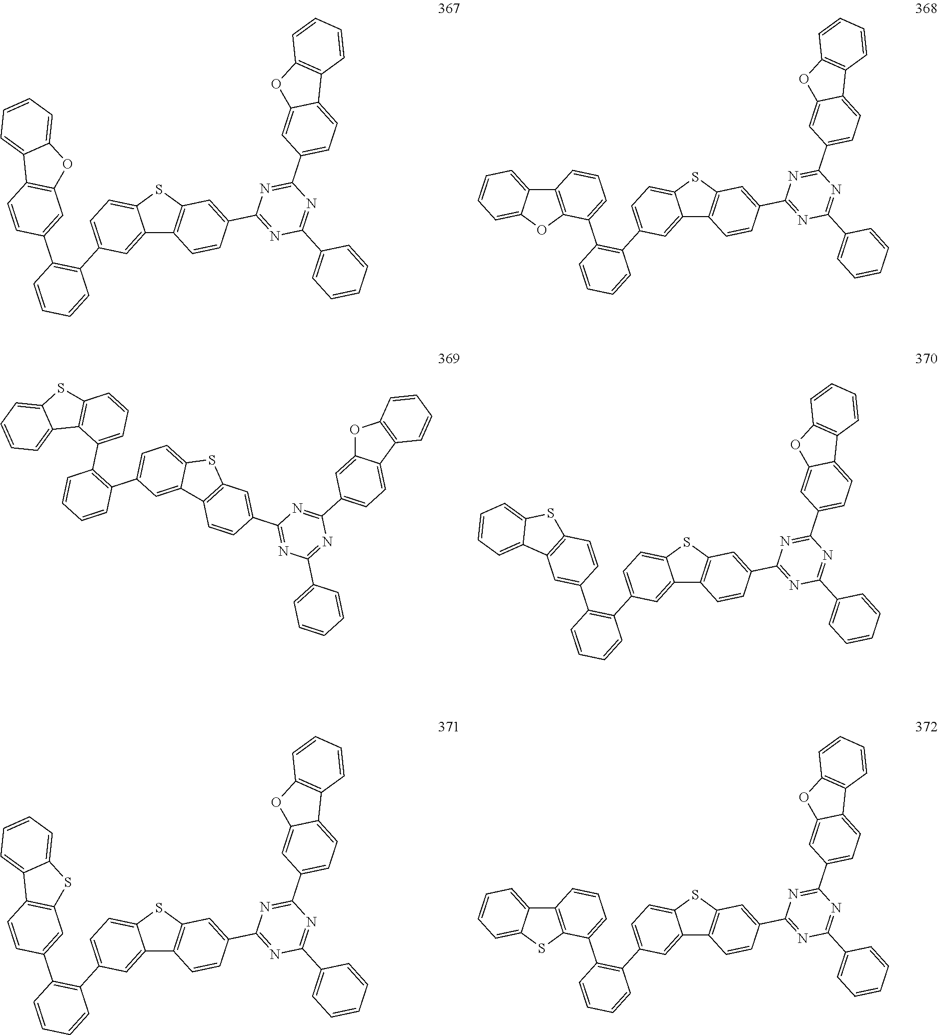

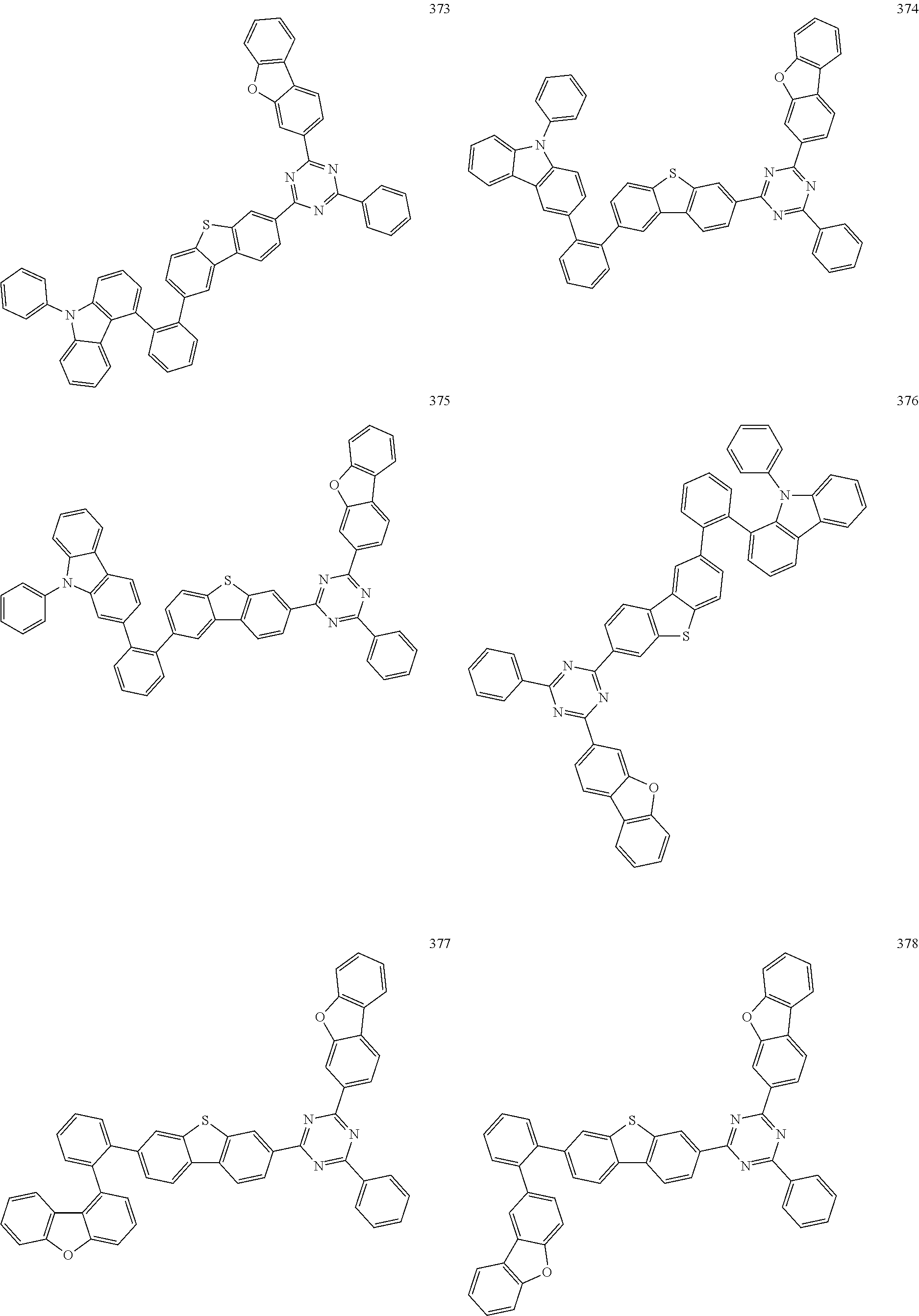

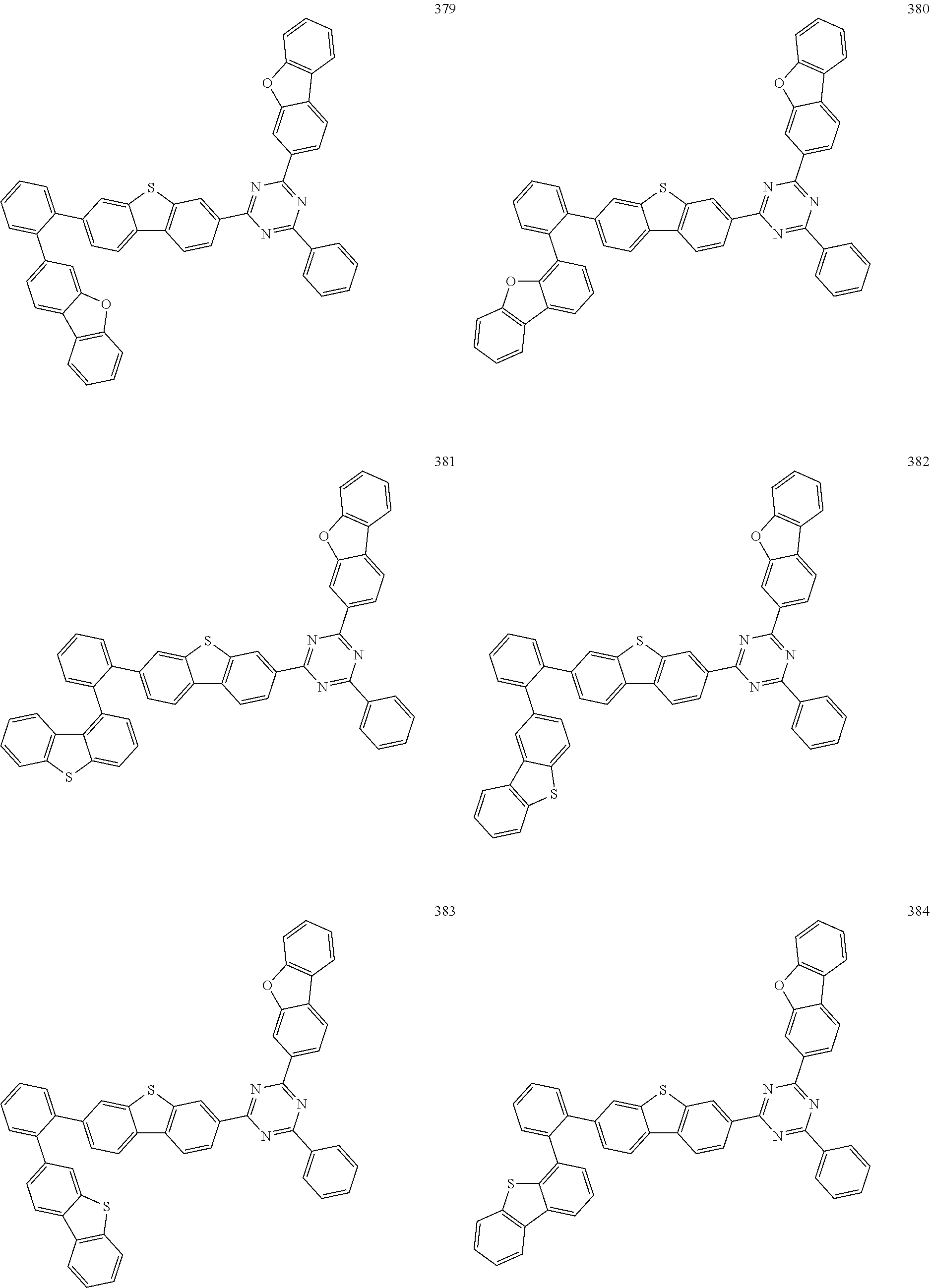

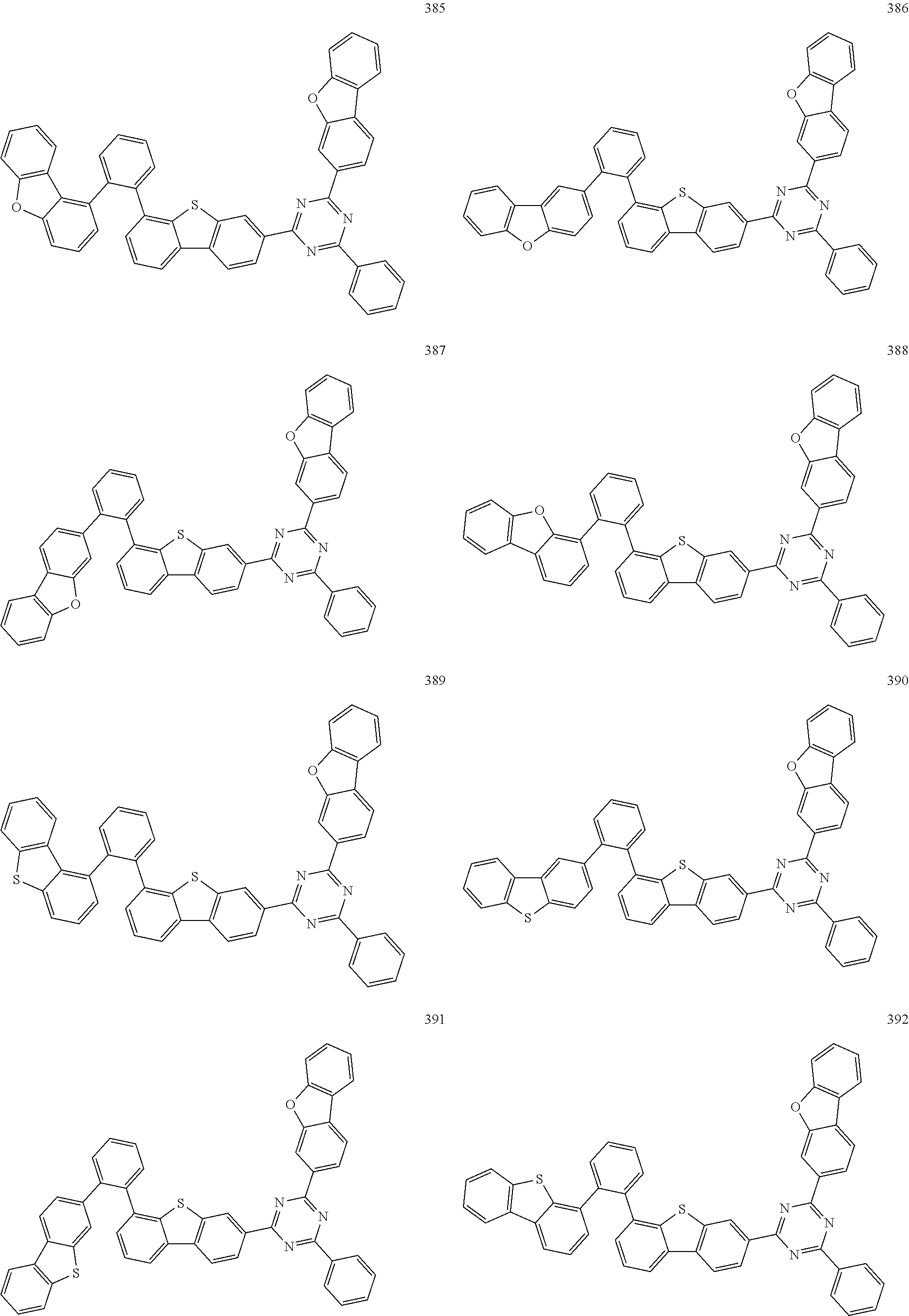

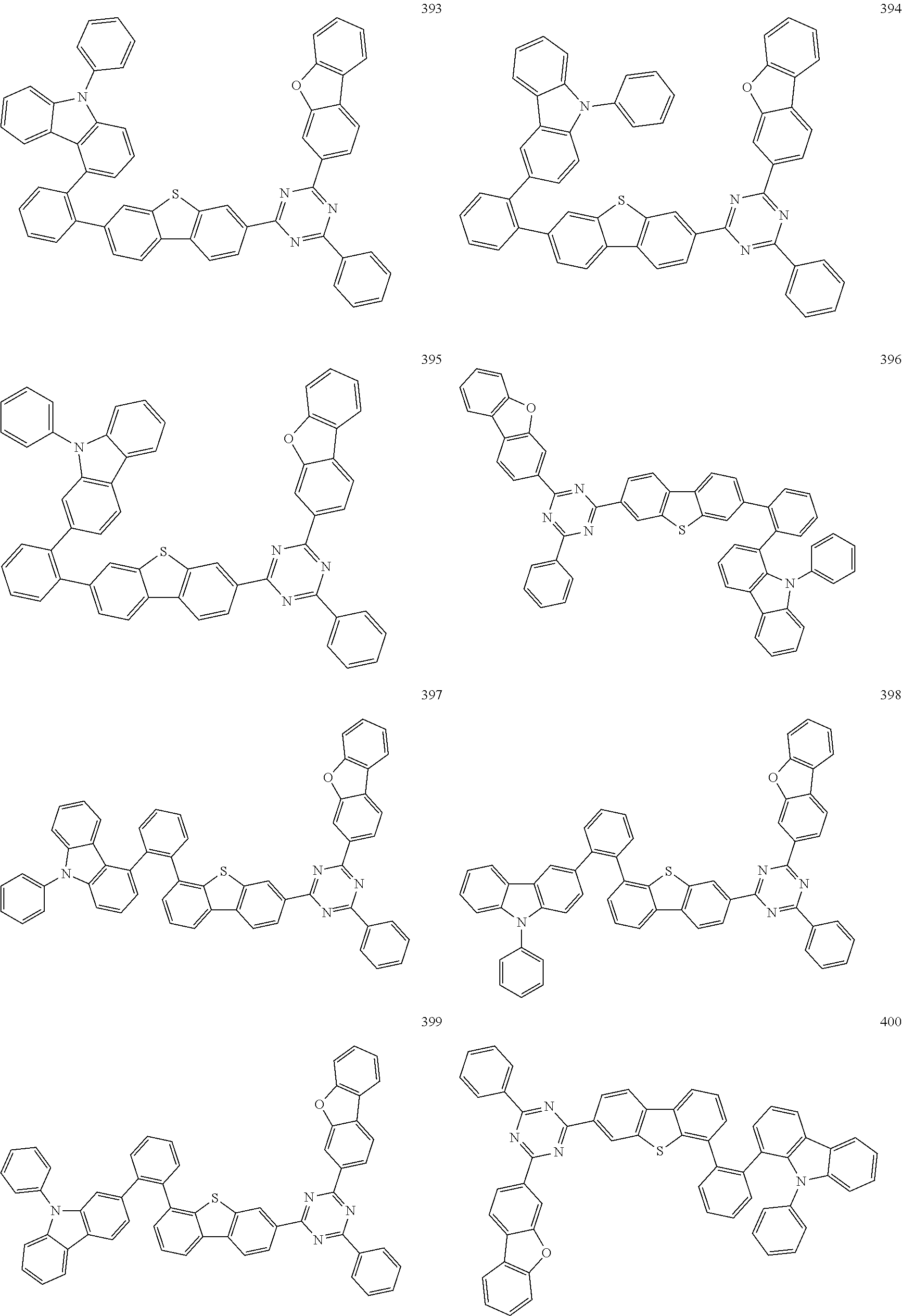

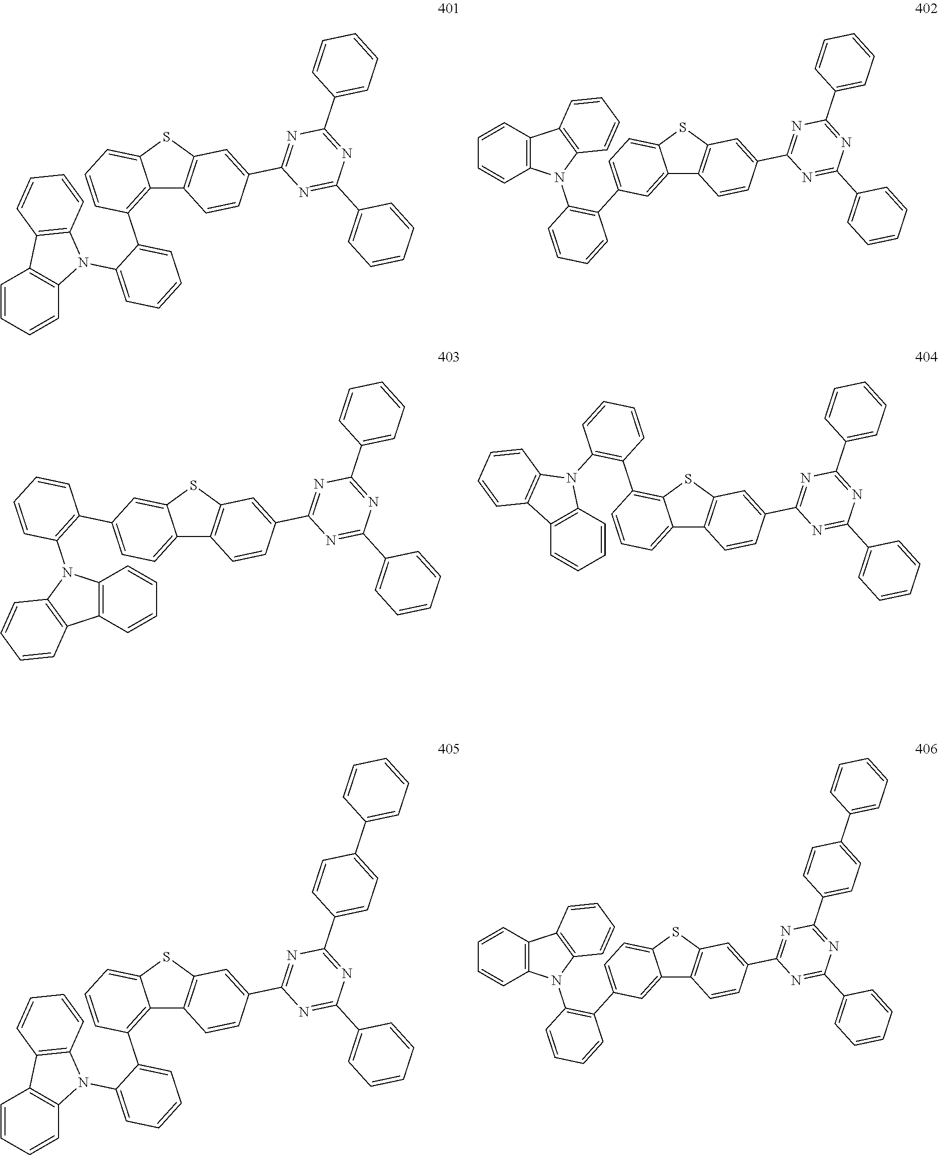

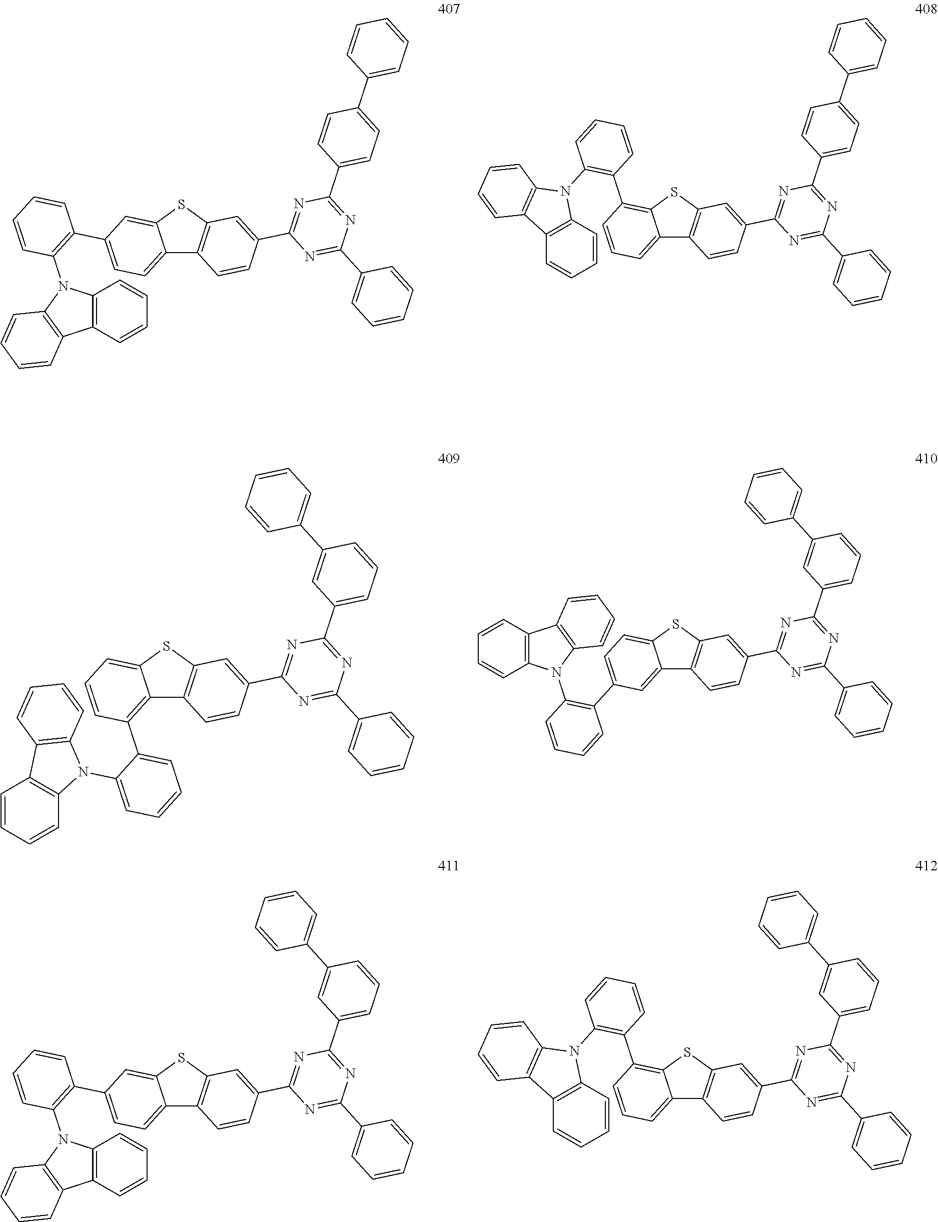

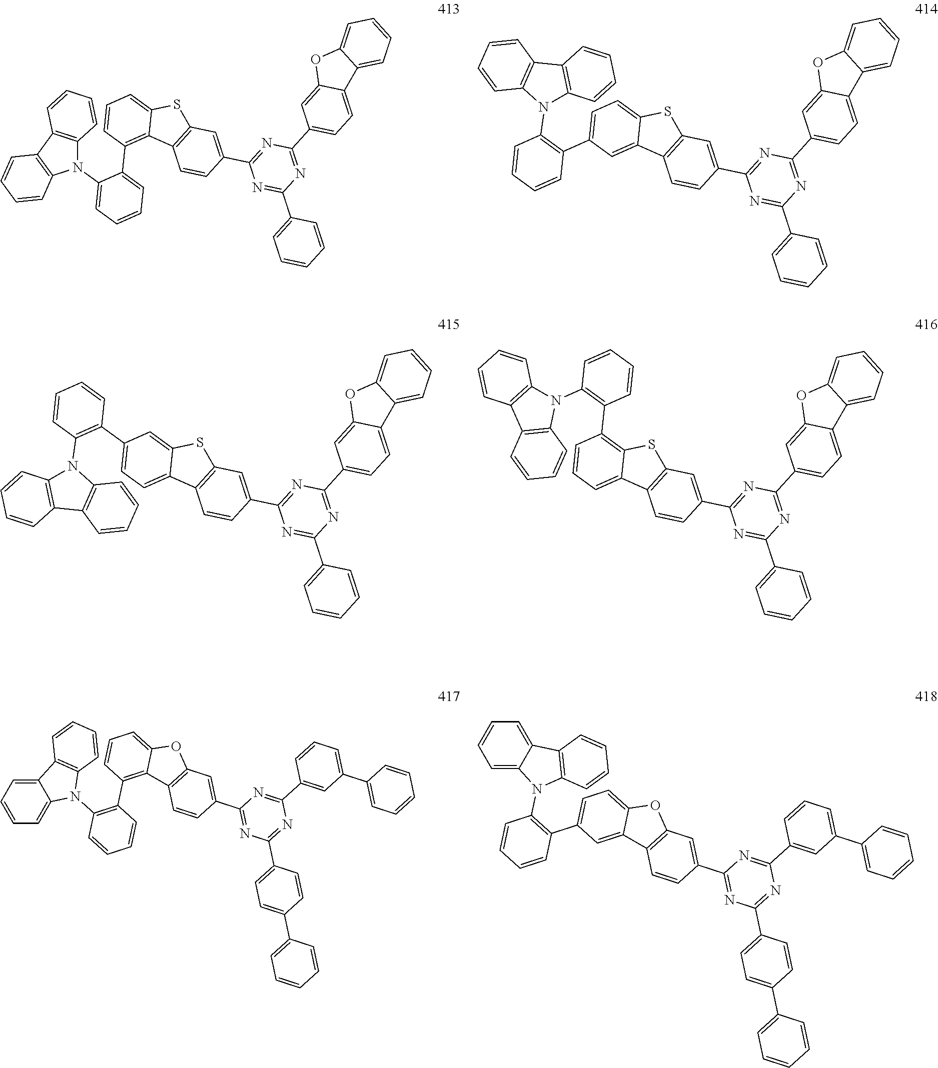

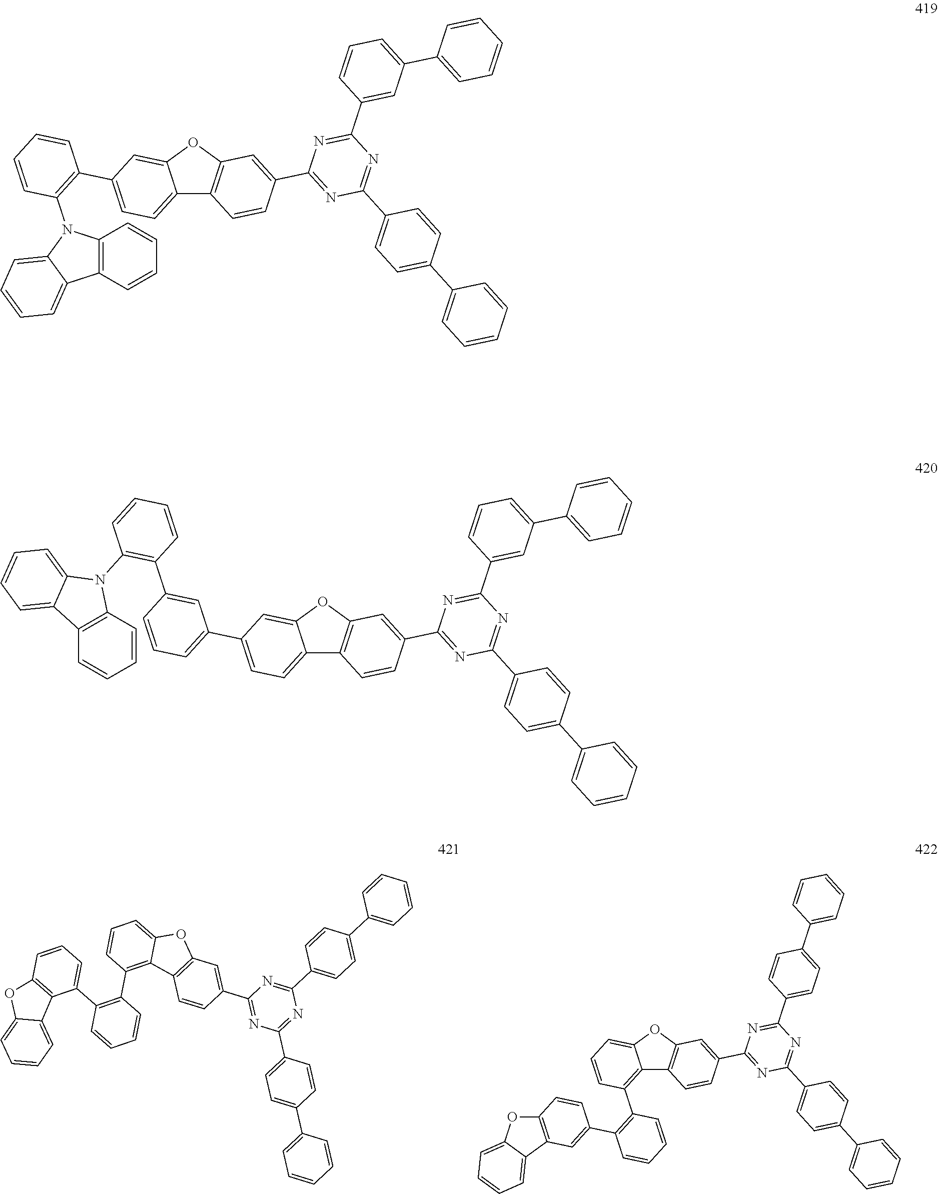 [SMILES: c1ccc(-c2ccc(-c3nc(-c4cccc(-c5ccccc5)c4)nc(-c4ccc5c(c4)oc4cc(-c6ccccc6-n6c7ccccc7c7ccccc76)ccc45)n3)cc2)cc1 ; c1ccc(-c2ccc(-c3nc(-c4cccc(-c5ccccc5)c4)nc(-c4ccc5c(c4)oc4cc(-c6cccc(-c7ccccc7-n7c8ccccc8c8ccccc87)c6)ccc45)n3)cc2)cc1 ; c1ccc(-c2ccc(-c3nc(-c4ccc(-c5ccccc5)cc4)nc(-c4ccc5c(c4)oc4cccc(-c6ccccc6-c6cccc7oc8ccccc8c67)c45)n3)cc2)cc1 ; c1ccc(-c2ccc(-c3nc(-c4ccc(-c5ccccc5)cc4)nc(-c4ccc5c(c4)oc4cccc(-c6ccccc6-c6ccc7oc8ccccc8c7c6)c45)n3)cc2)cc1]

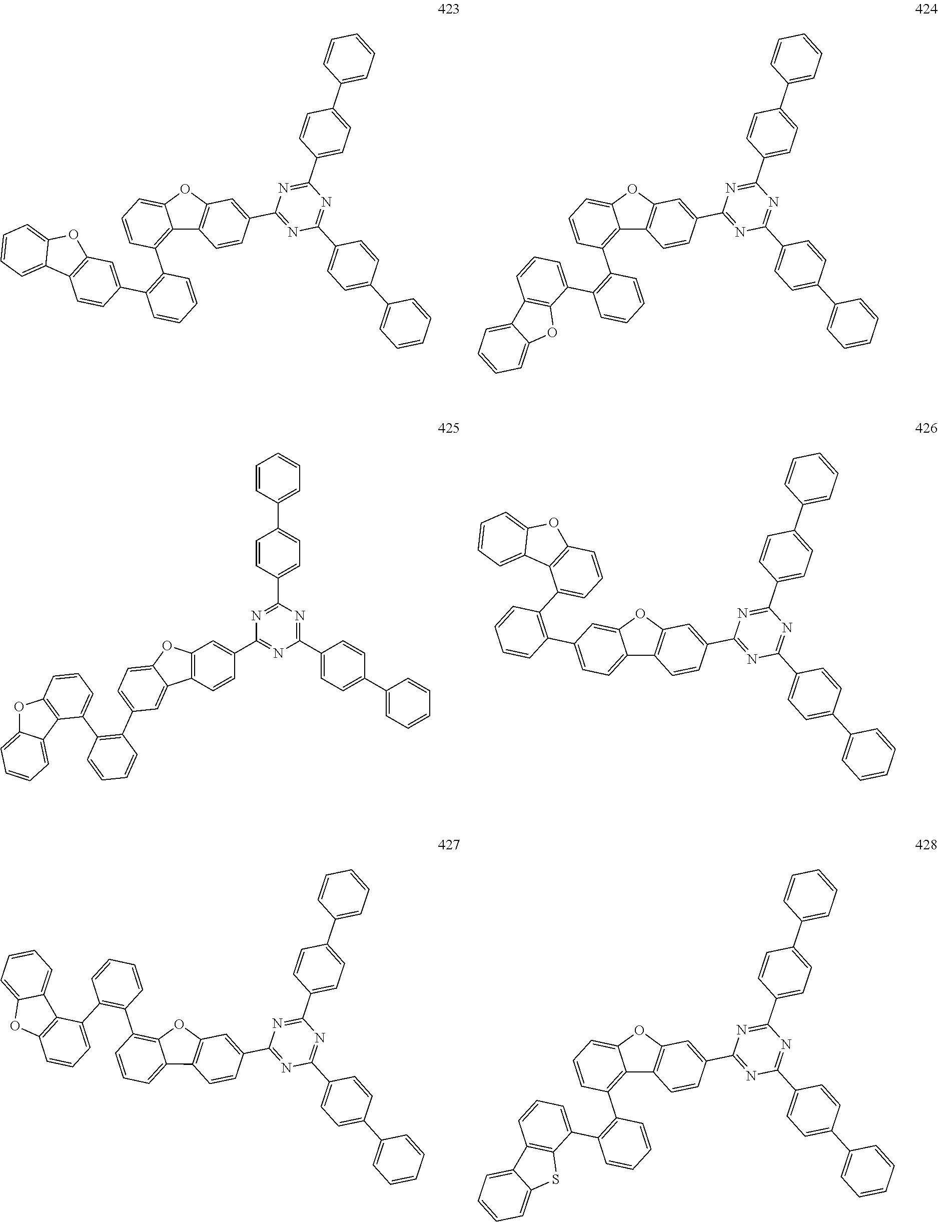

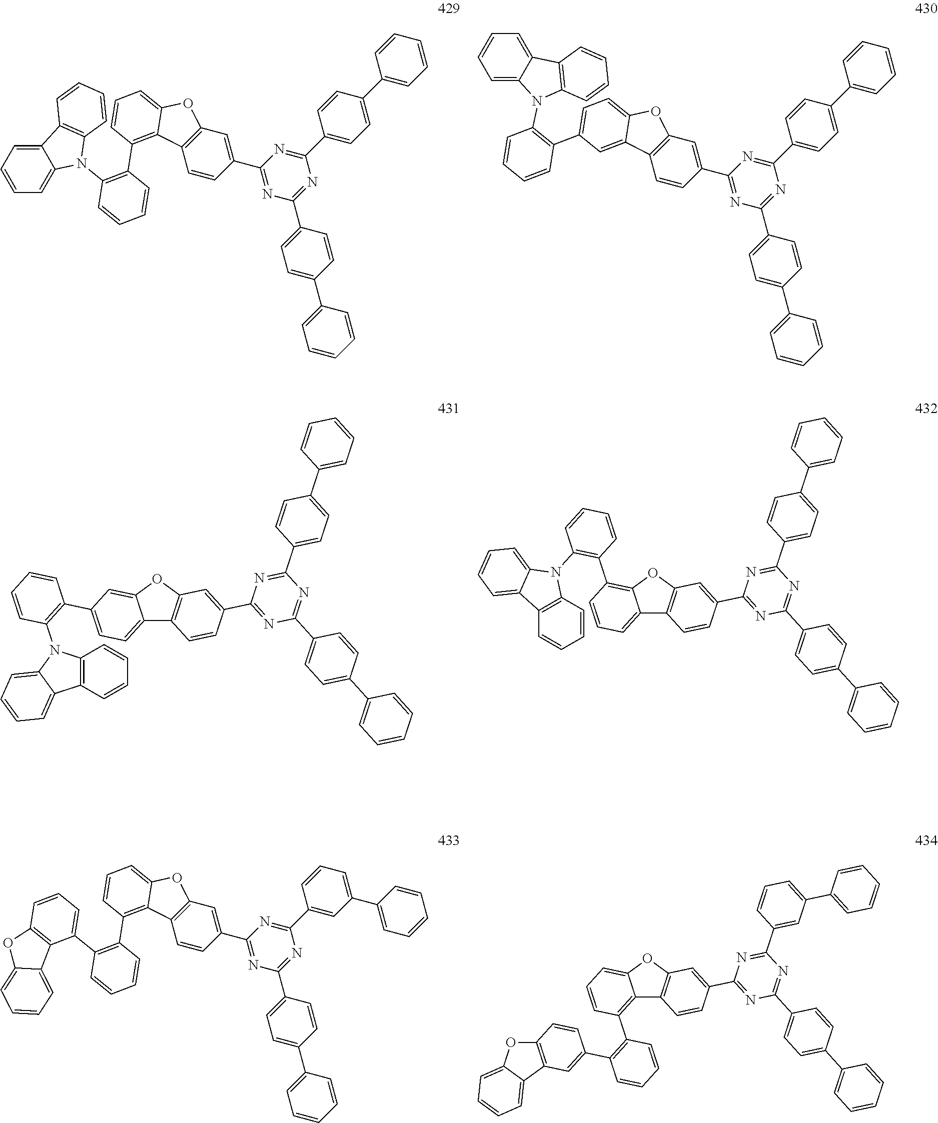

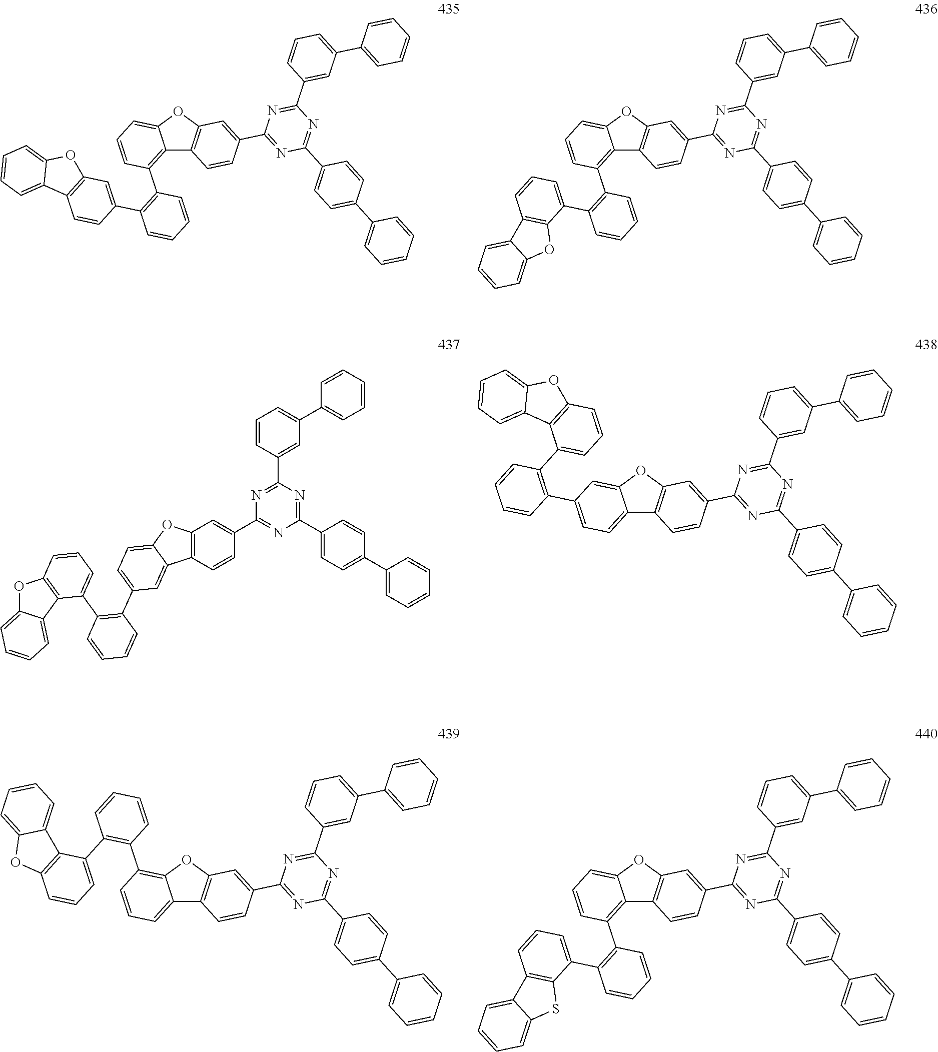

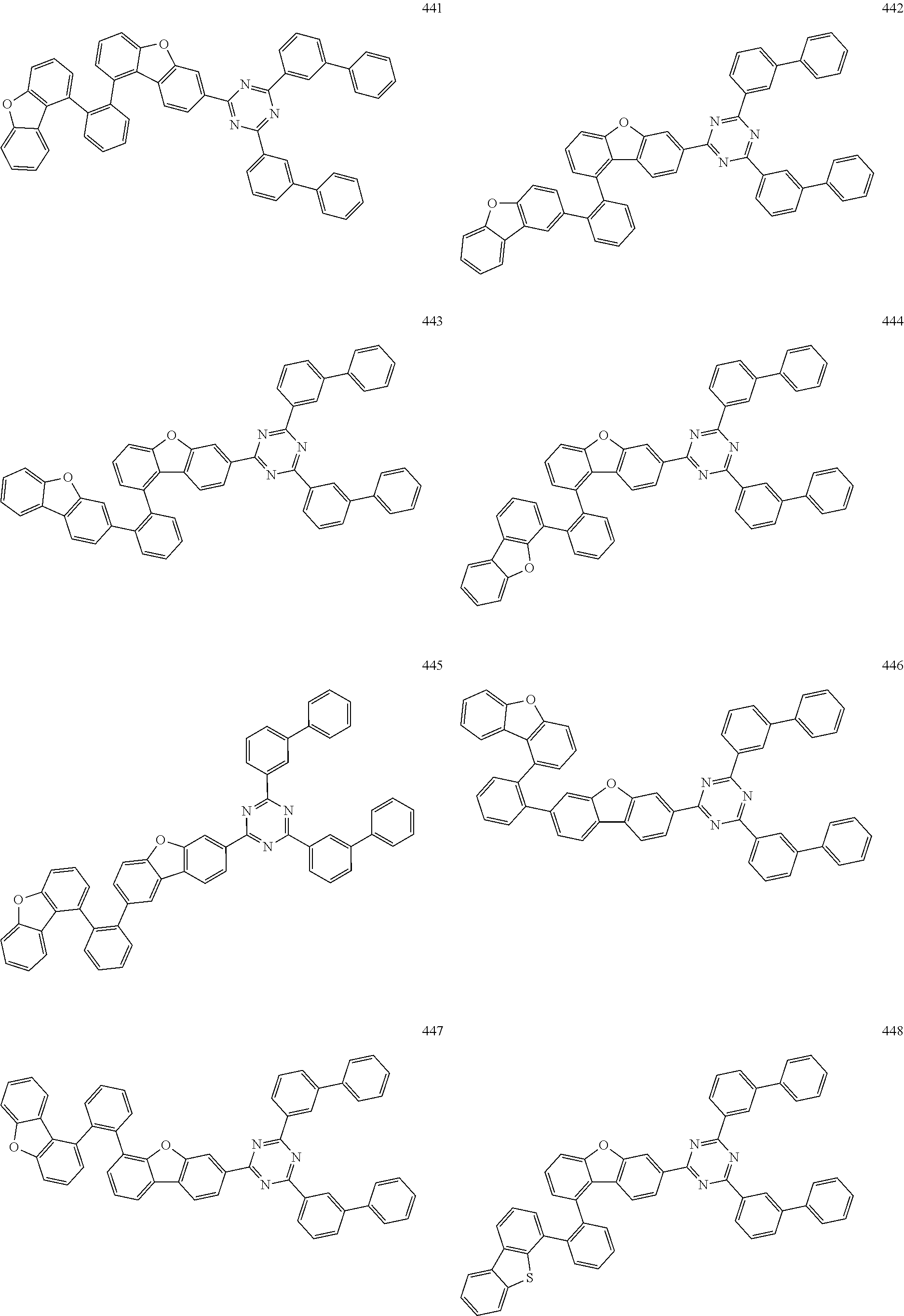

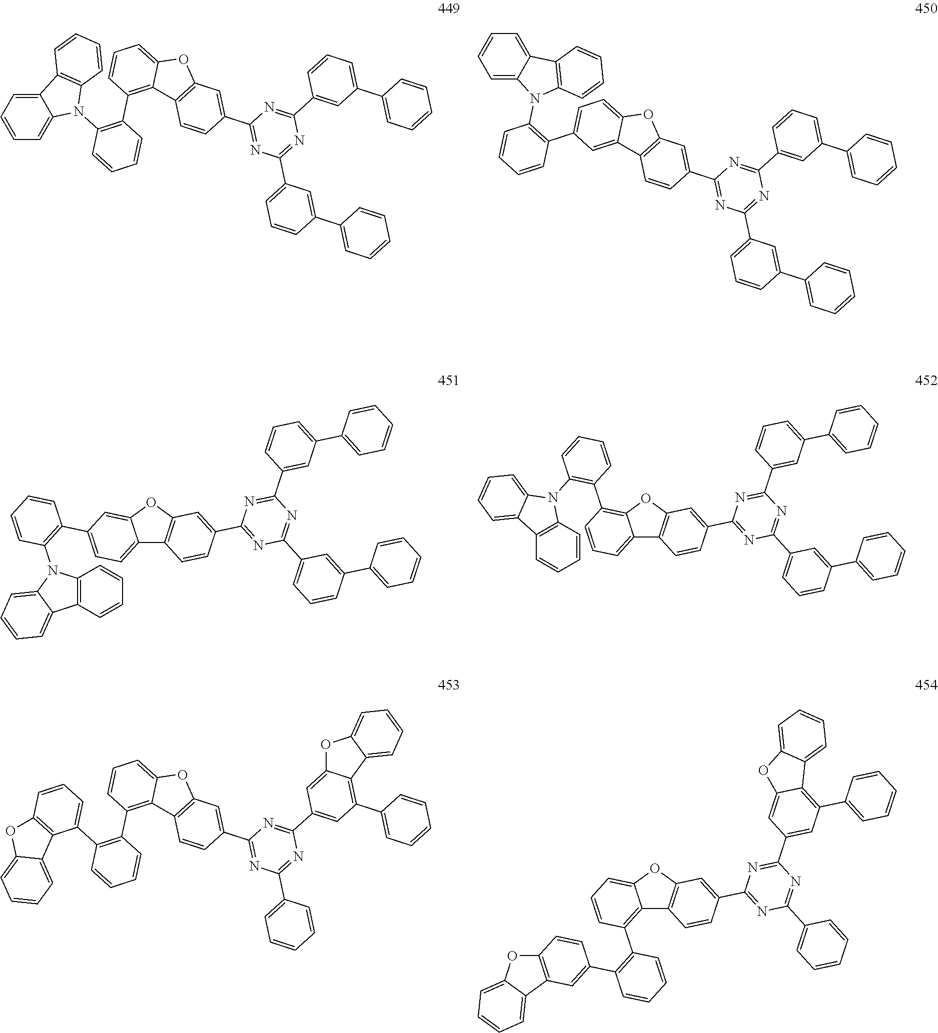

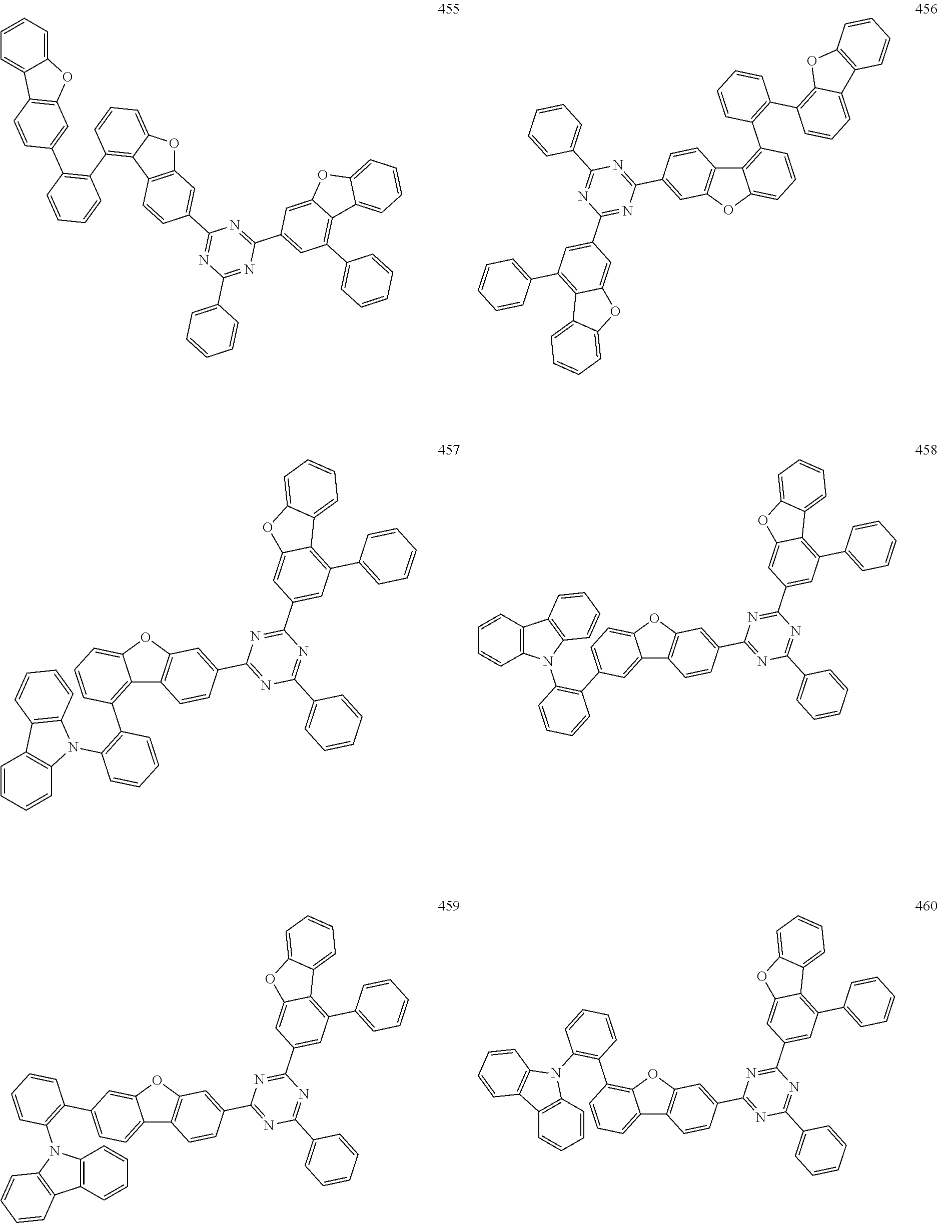

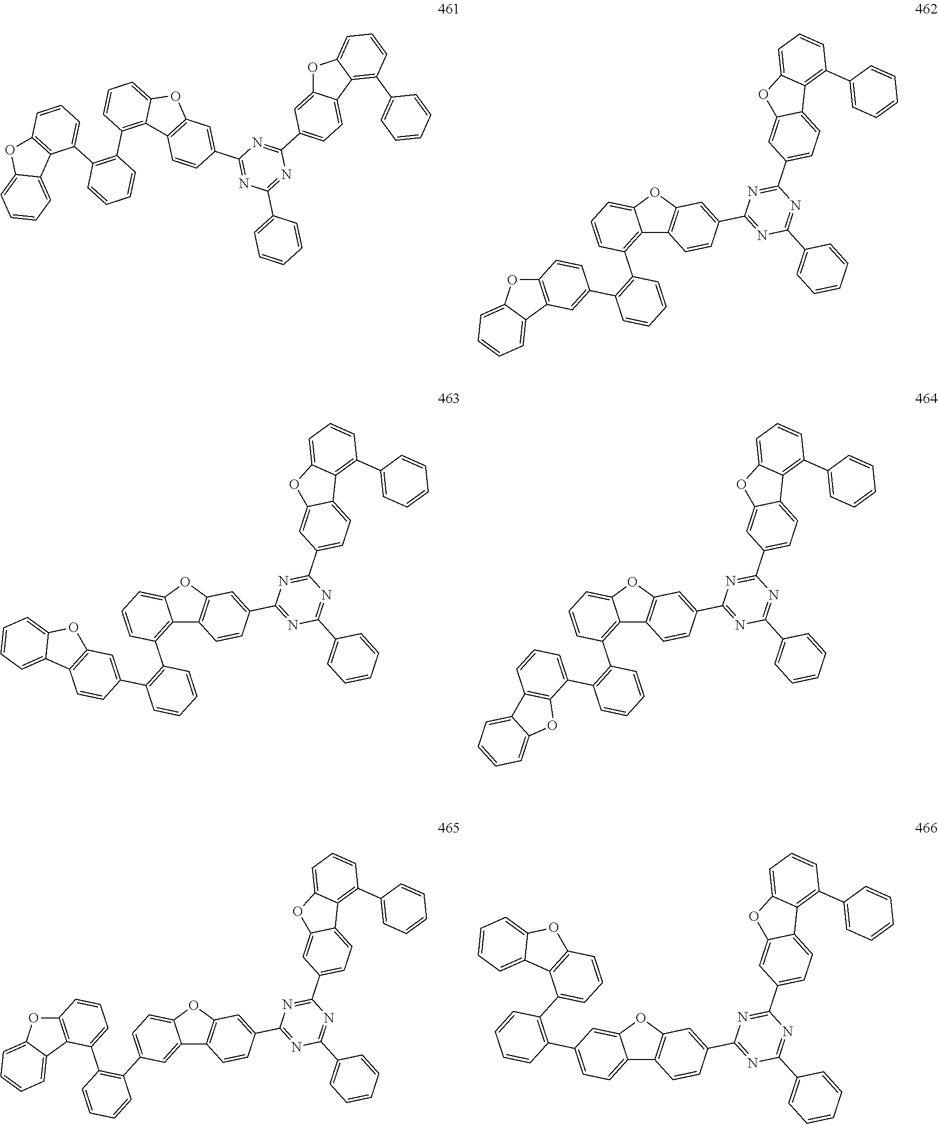

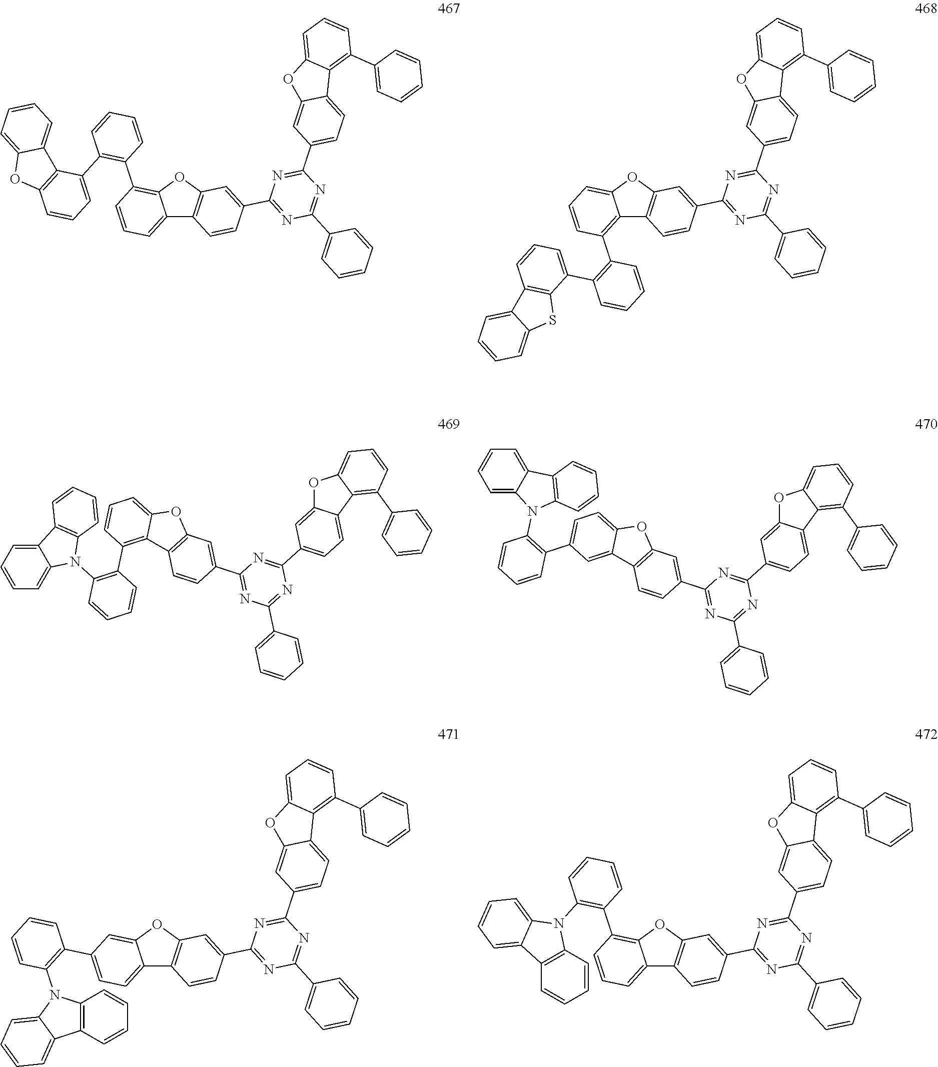

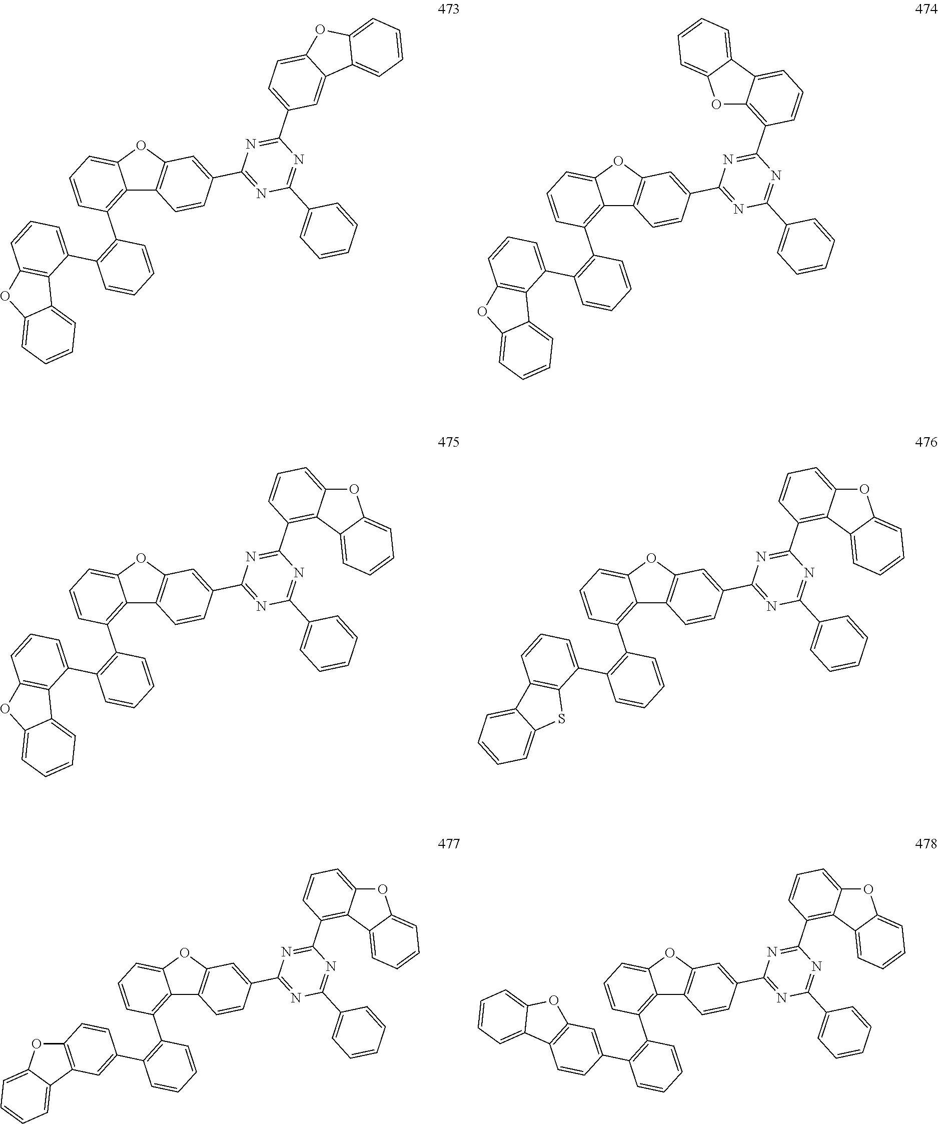

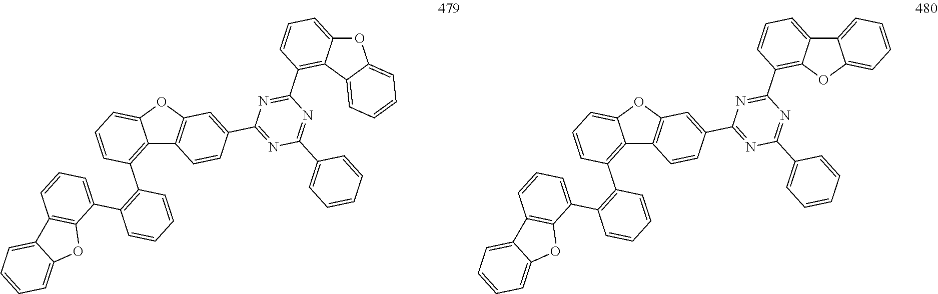 [SMILES: c1ccc(-c2nc(-c3ccc4c(c3)oc3cccc(-c5ccccc5-c5cccc6c5oc5ccccc56)c34)nc(-c3cccc4oc5ccccc5c34)n2)cc1 ; c1ccc(-c2nc(-c3ccc4c(c3)oc3cccc(-c5ccccc5-c5cccc6c5oc5ccccc56)c34)nc(-c3cccc4c3oc3ccccc34)n2)cc1]

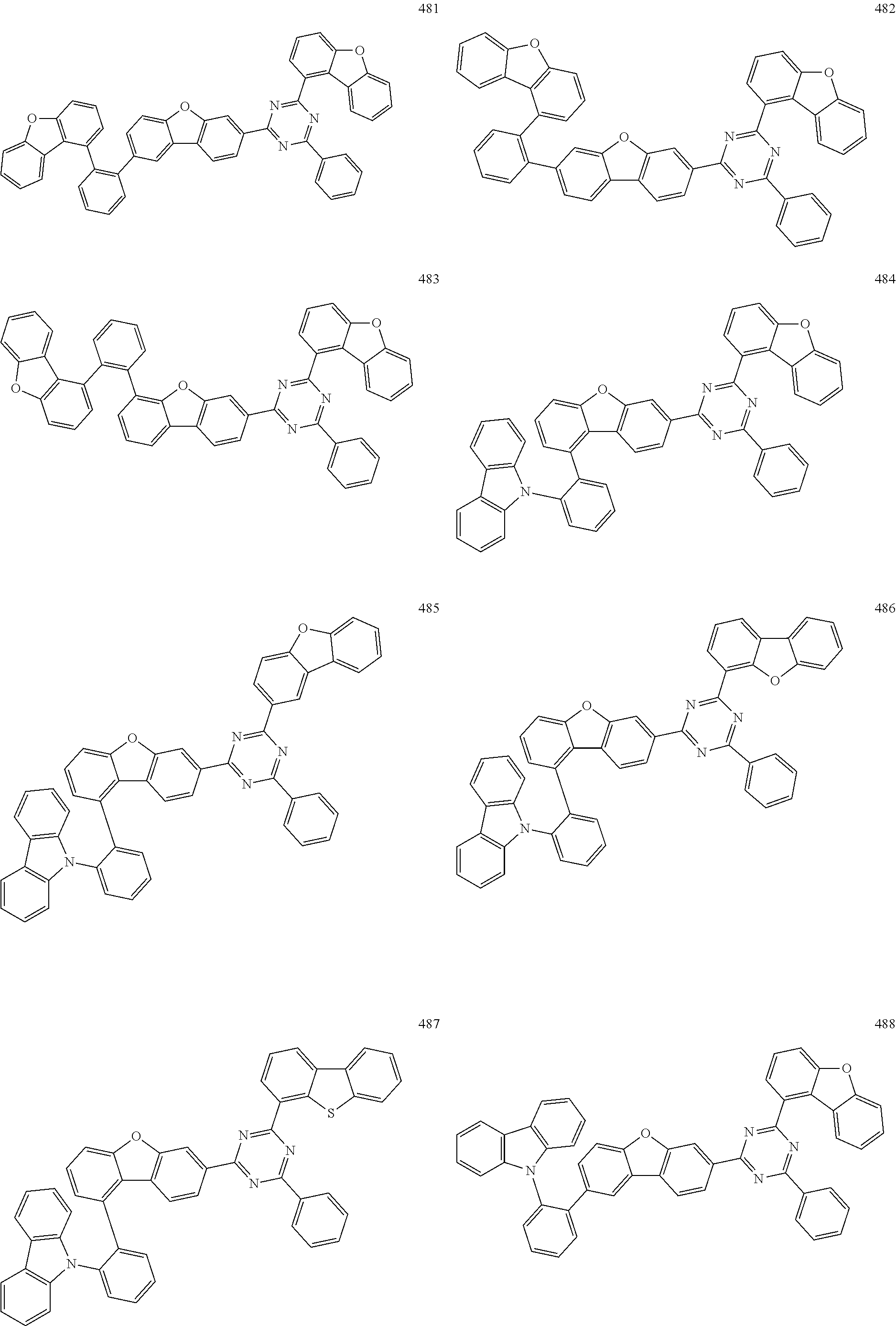

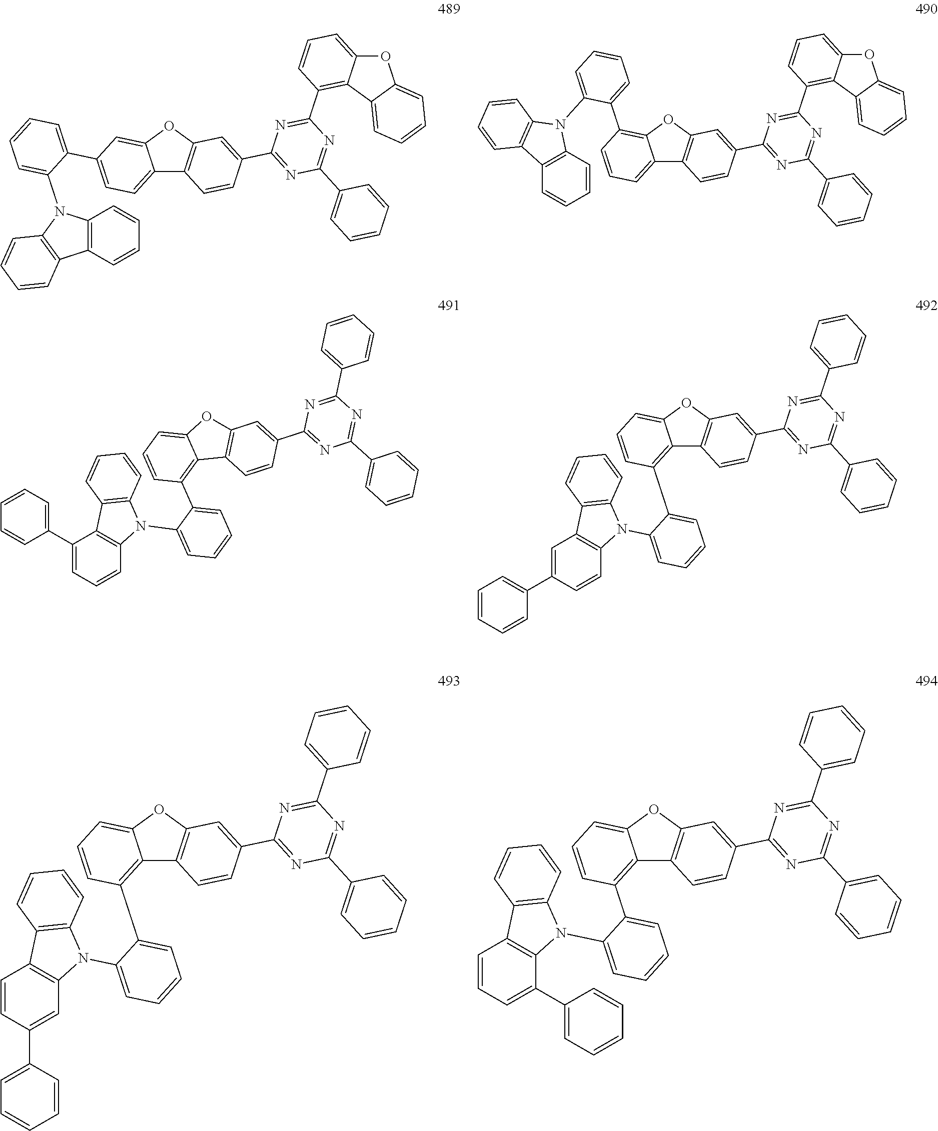

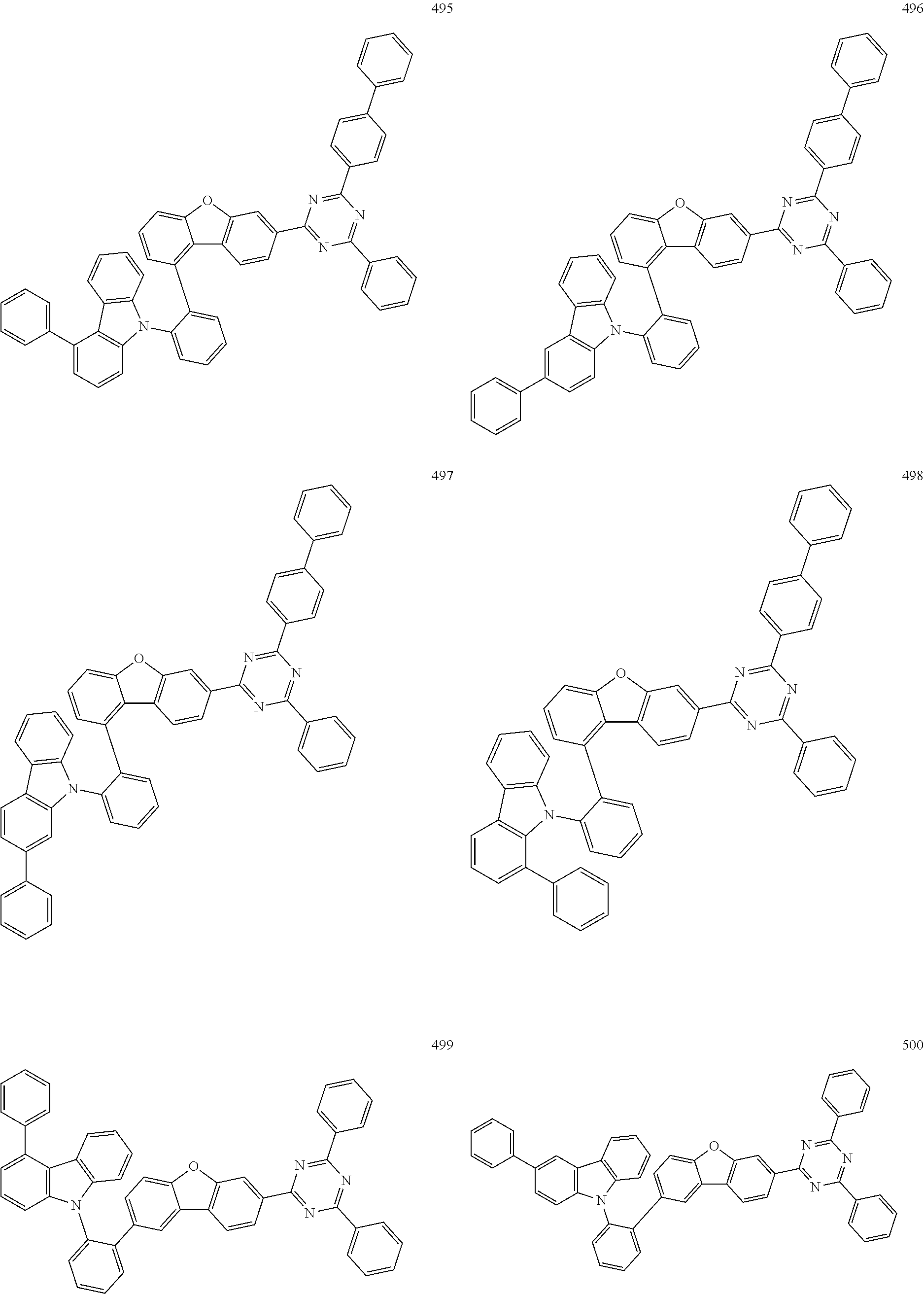

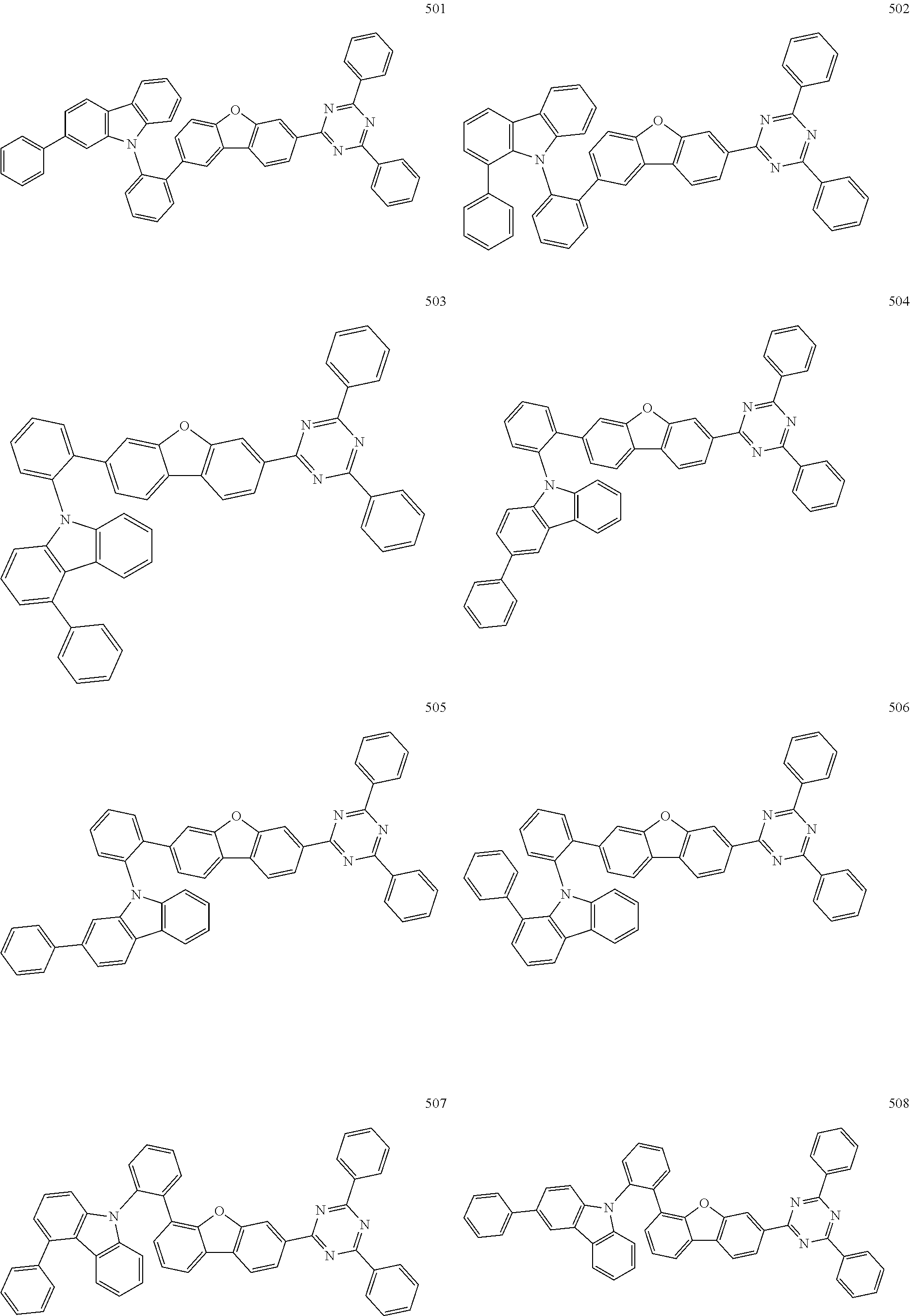

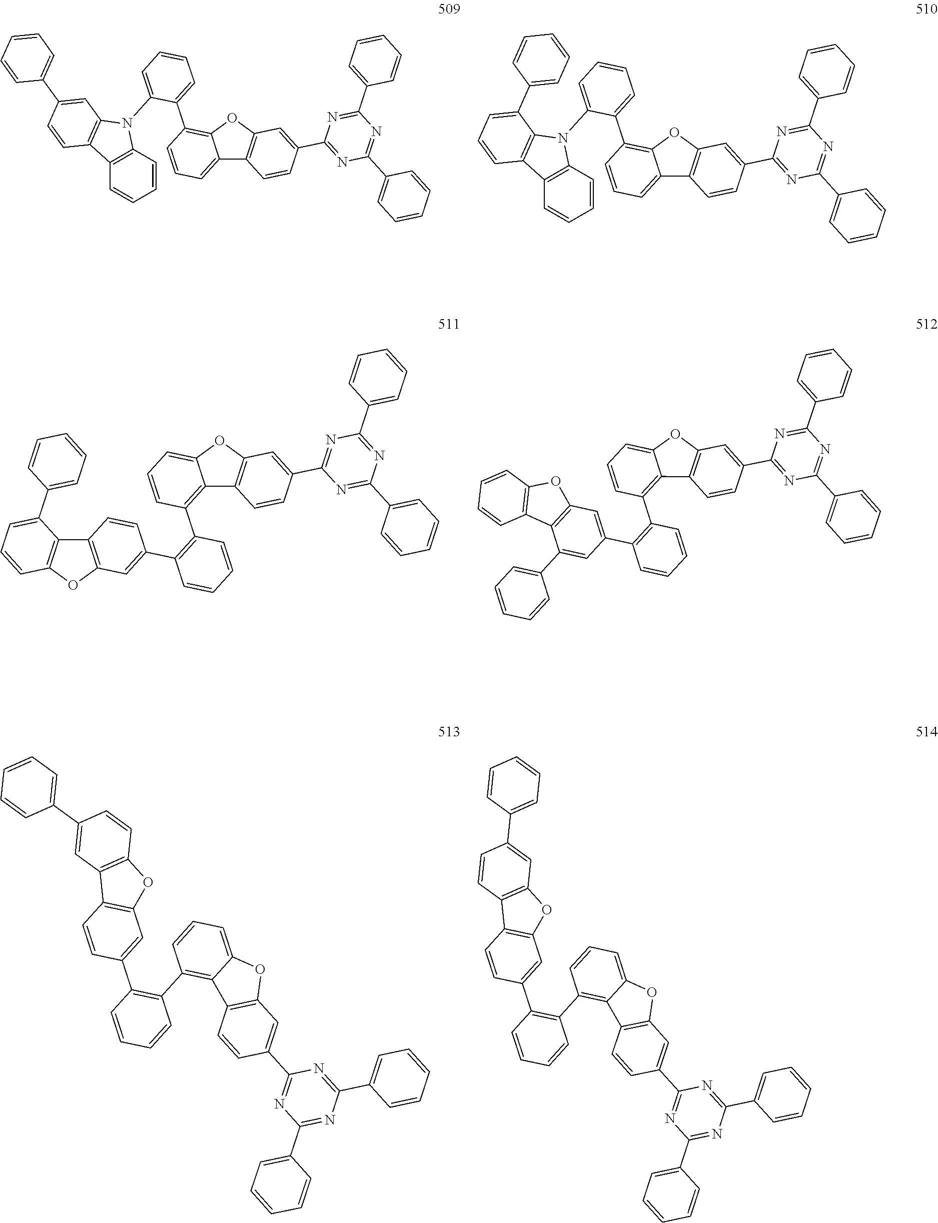

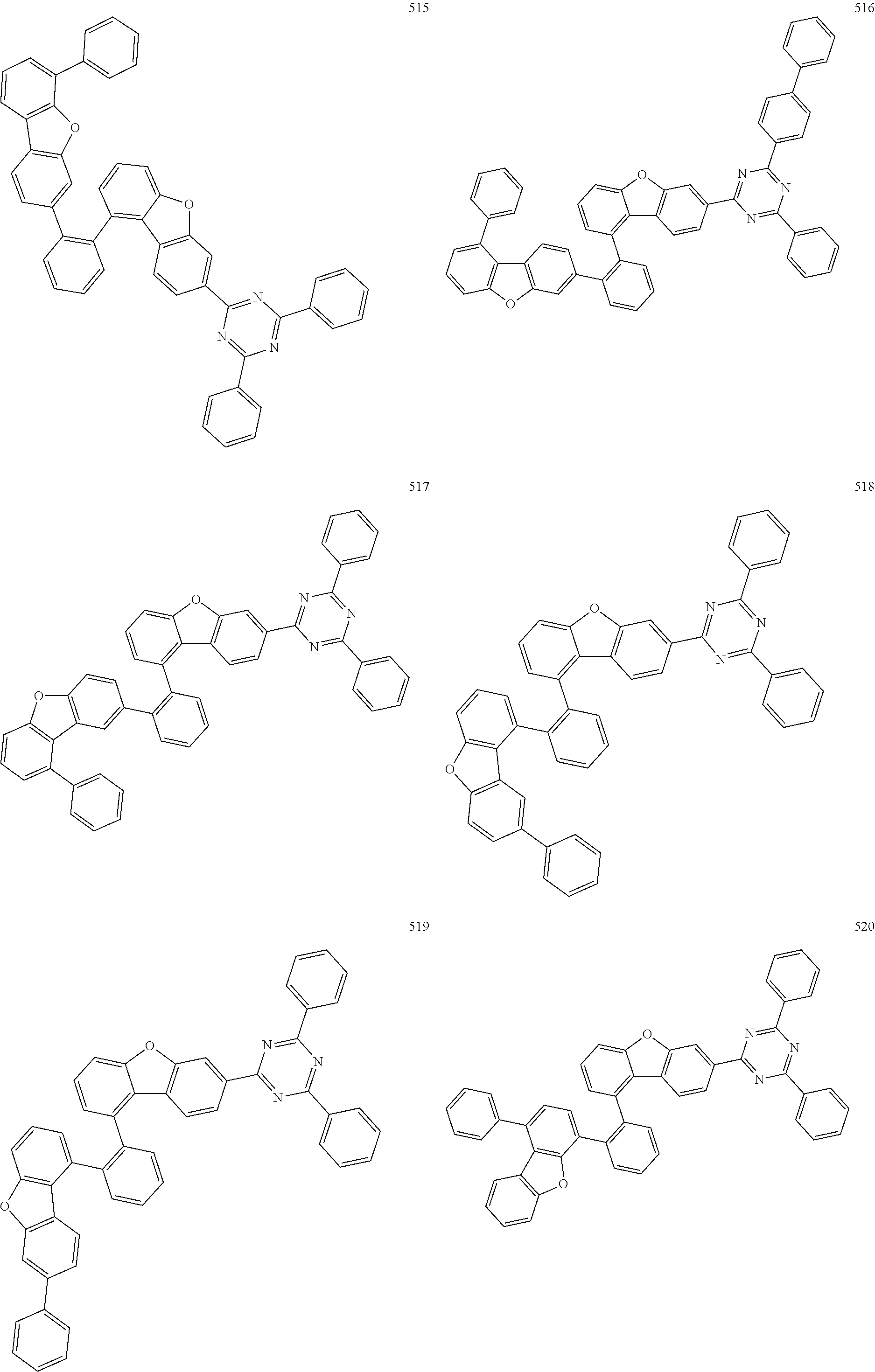

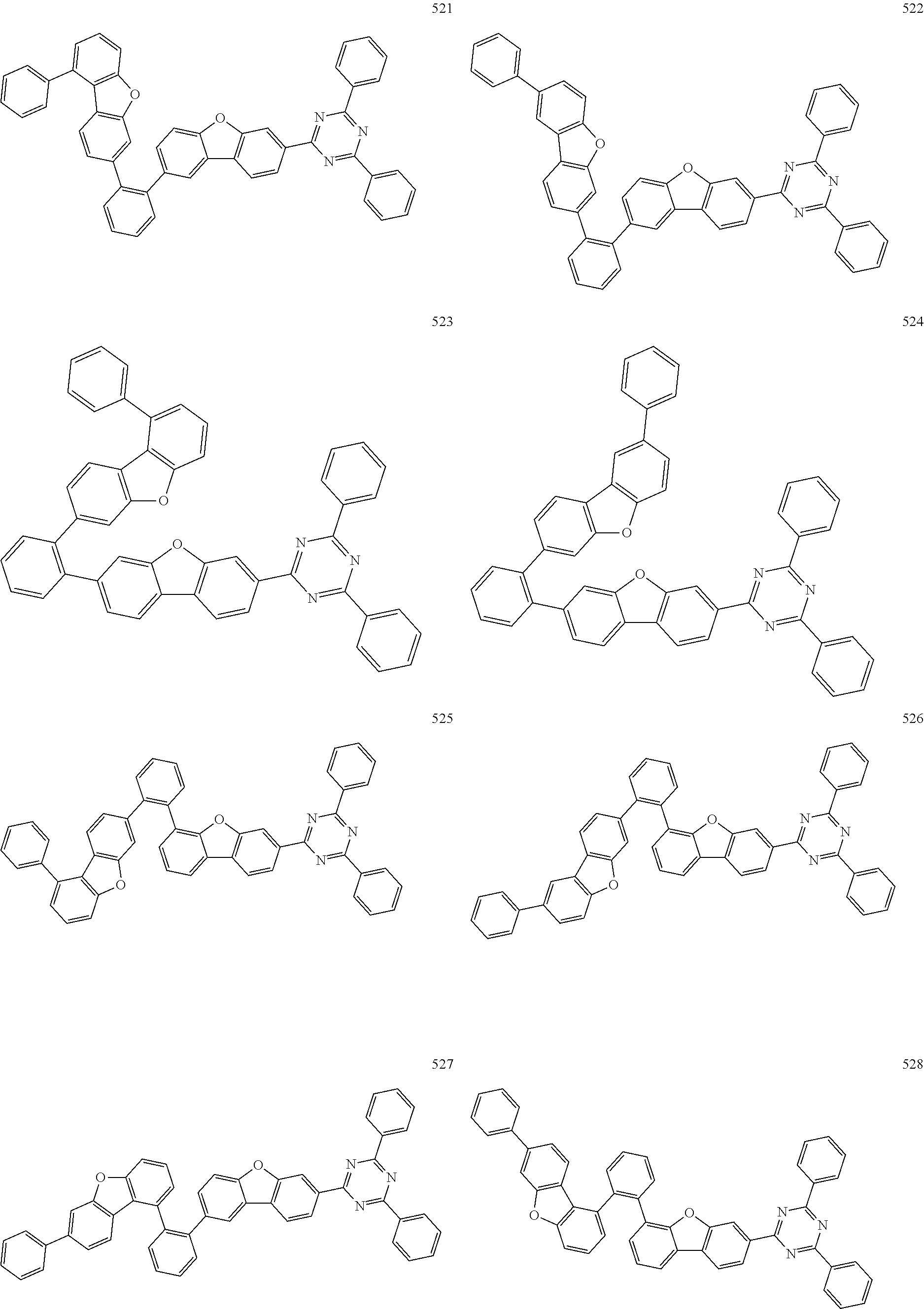

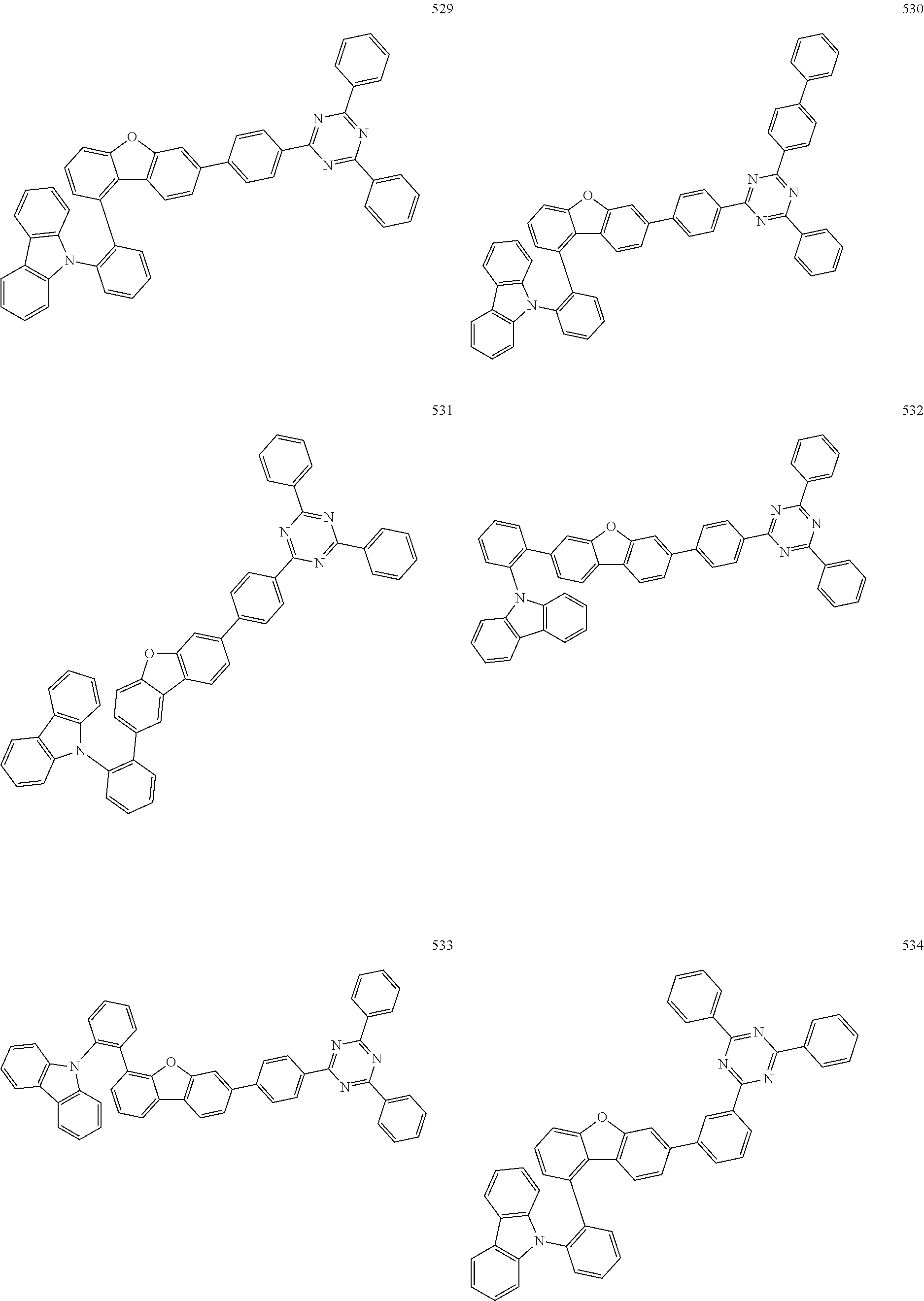

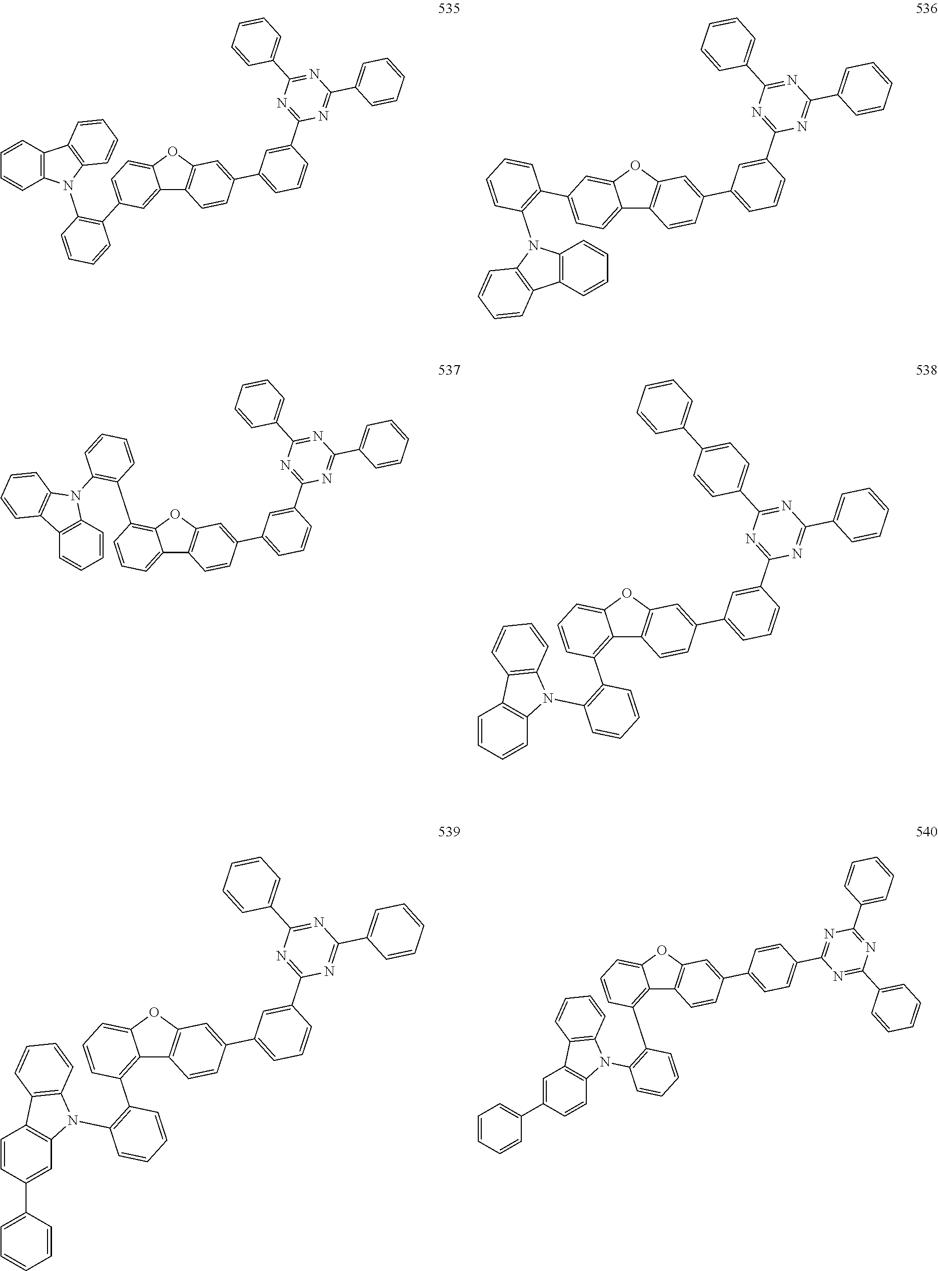

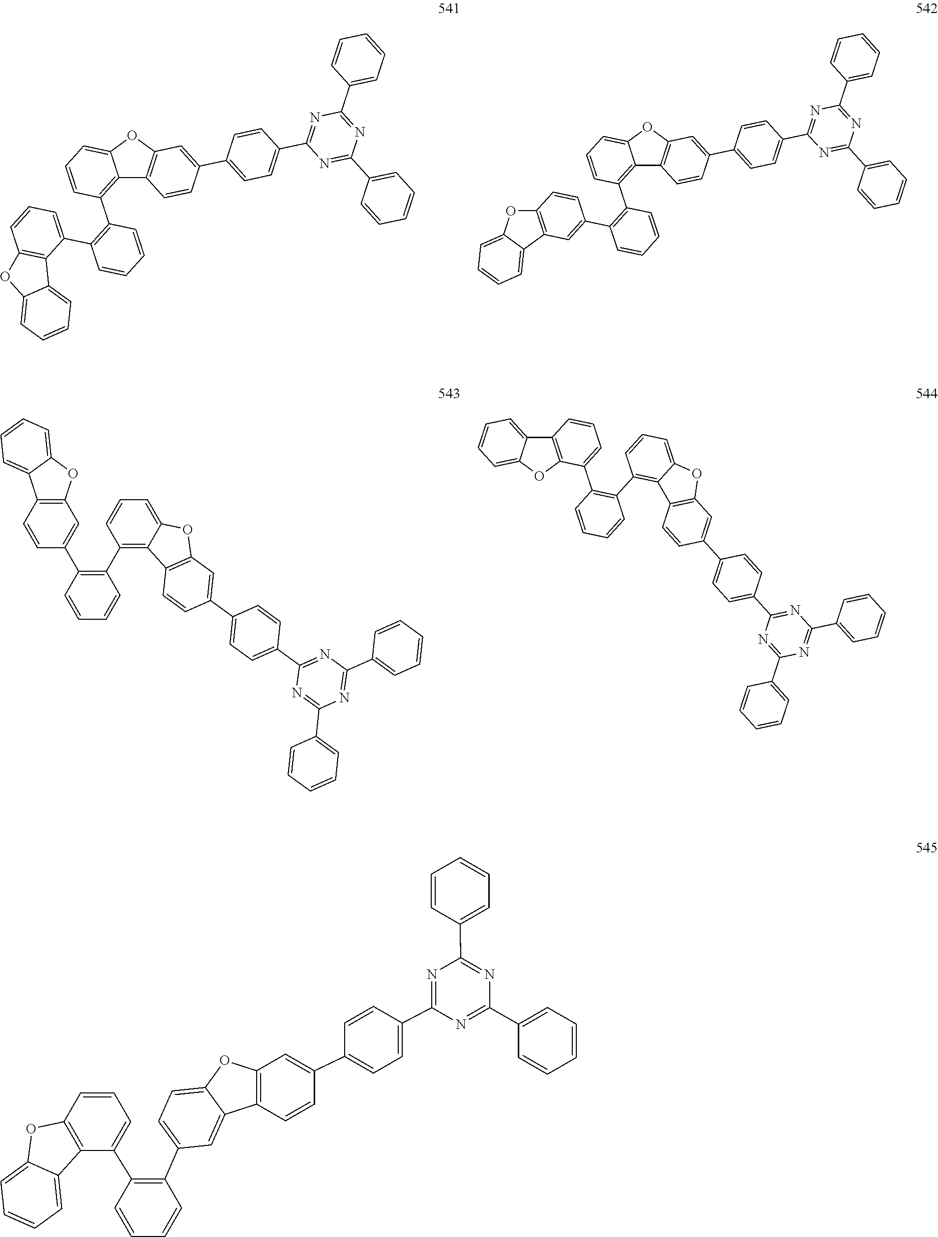 [SMILES: c1ccc(-c2nc(-c3ccccc3)nc(-c3ccc(-c4ccc5c(c4)oc4cccc(-c6ccccc6-c6cccc7oc8ccccc8c67)c45)cc3)n2)cc1 ; c1ccc(-c2nc(-c3ccccc3)nc(-c3ccc(-c4ccc5c(c4)oc4cccc(-c6ccccc6-c6ccc7oc8ccccc8c7c6)c45)cc3)n2)cc1 ; c1ccc(-c2nc(-c3ccccc3)nc(-c3ccc(-c4ccc5c(c4)oc4cccc(-c6ccccc6-c6ccc7c(c6)oc6ccccc67)c45)cc3)n2)cc1 ; c1ccc(-c2nc(-c3ccccc3)nc(-c3ccc(-c4ccc5c(c4)oc4cccc(-c6ccccc6-c6cccc7c6oc6ccccc67)c45)cc3)n2)cc1 ; c1ccc(-c2nc(-c3ccccc3)nc(-c3ccc(-c4ccc5c(c4)oc4ccc(-c6ccccc6-c6cccc7oc8ccccc8c67)cc45)cc3)n2)cc1]

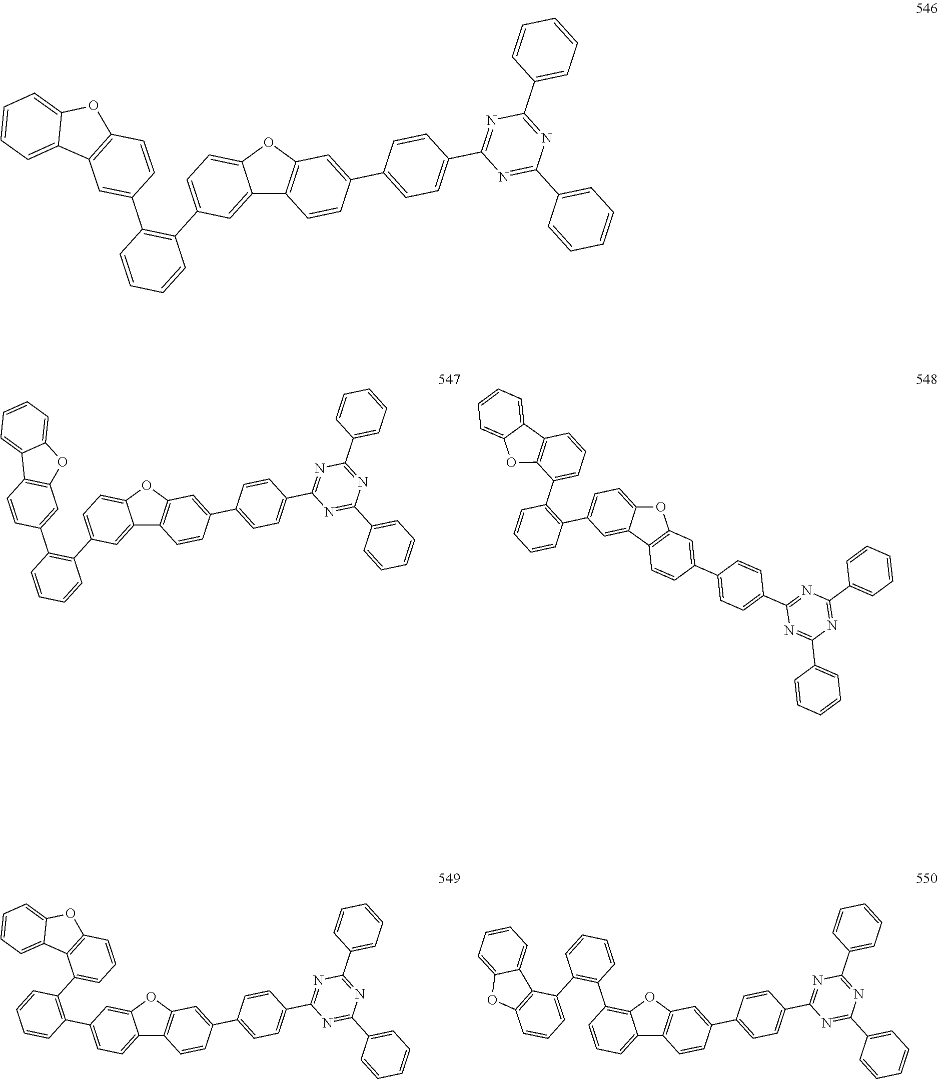 [SMILES: c1ccc(-c2nc(-c3ccccc3)nc(-c3ccc(-c4ccc5c(c4)oc4ccc(-c6ccccc6-c6ccc7oc8ccccc8c7c6)cc45)cc3)n2)cc1 ; c1ccc(-c2nc(-c3ccccc3)nc(-c3ccc(-c4ccc5c(c4)oc4ccc(-c6ccccc6-c6ccc7c(c6)oc6ccccc67)cc45)cc3)n2)cc1 ; c1ccc(-c2nc(-c3ccccc3)nc(-c3ccc(-c4ccc5c(c4)oc4ccc(-c6ccccc6-c6cccc7c6oc6ccccc67)cc45)cc3)n2)cc1 ; c1ccc(-c2nc(-c3ccccc3)nc(-c3ccc(-c4ccc5c(c4)oc4cc(-c6ccccc6-c6cccc7oc8ccccc8c67)ccc45)cc3)n2)cc1 ; c1ccc(-c2nc(-c3ccccc3)nc(-c3ccc(-c4ccc5c(c4)oc4c(-c6ccccc6-c6cccc7oc8ccccc8c67)cccc45)cc3)n2)cc1]

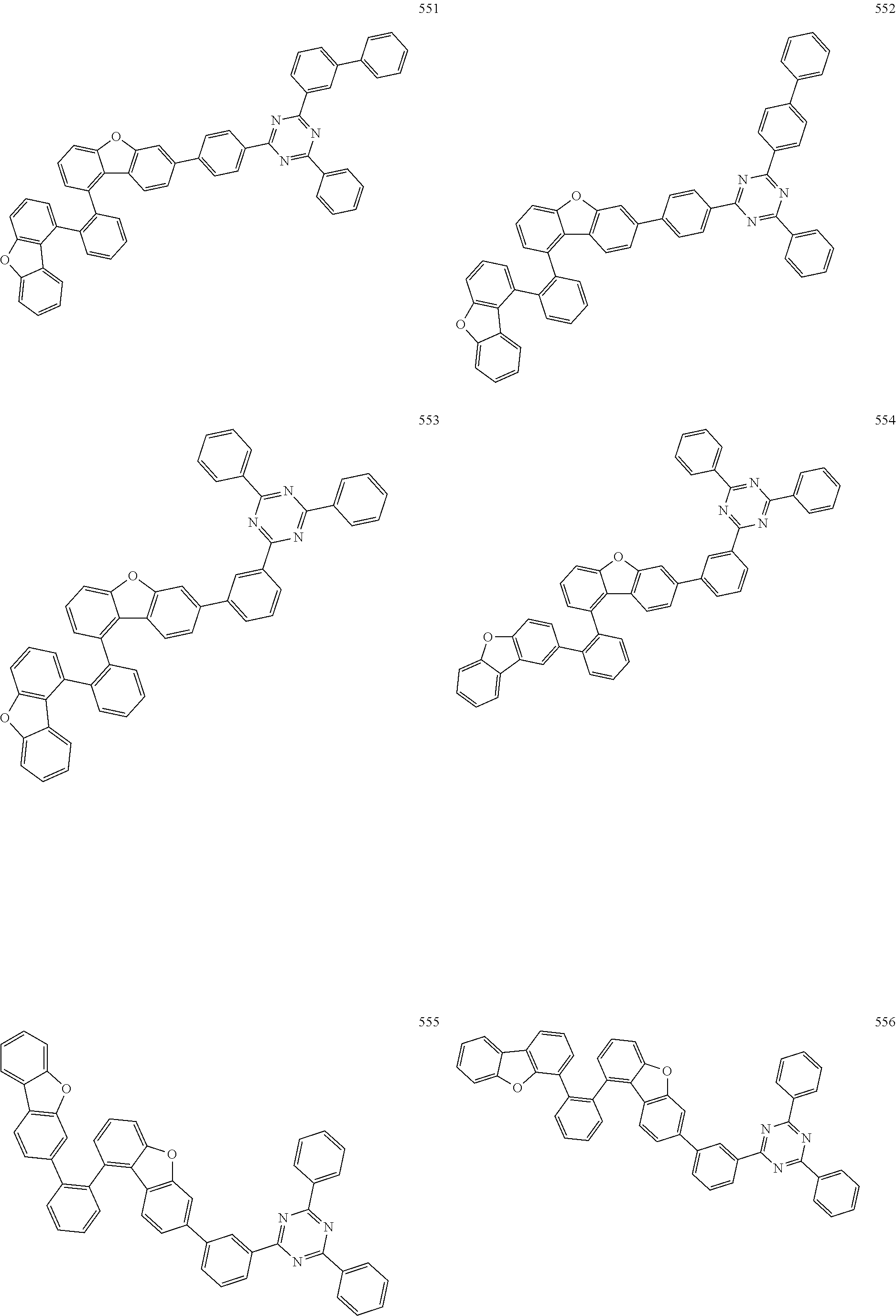

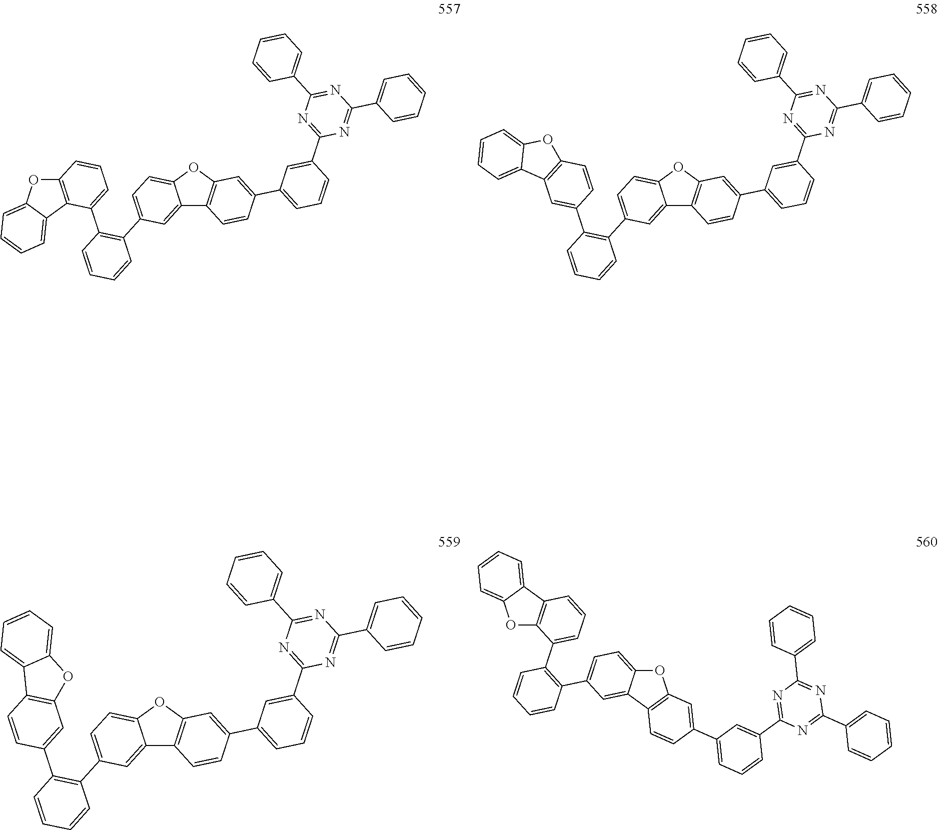 [SMILES: c1ccc(-c2nc(-c3ccccc3)nc(-c3cccc(-c4ccc5c(c4)oc4ccc(-c6ccccc6-c6cccc7oc8ccccc8c67)cc45)c3)n2)cc1 ; c1ccc(-c2nc(-c3ccccc3)nc(-c3cccc(-c4ccc5c(c4)oc4ccc(-c6ccccc6-c6ccc7oc8ccccc8c7c6)cc45)c3)n2)cc1 ; c1ccc(-c2nc(-c3ccccc3)nc(-c3cccc(-c4ccc5c(c4)oc4ccc(-c6ccccc6-c6ccc7c(c6)oc6ccccc67)cc45)c3)n2)cc1 ; c1ccc(-c2nc(-c3ccccc3)nc(-c3cccc(-c4ccc5c(c4)oc4ccc(-c6ccccc6-c6cccc7c6oc6ccccc67)cc45)c3)n2)cc1]

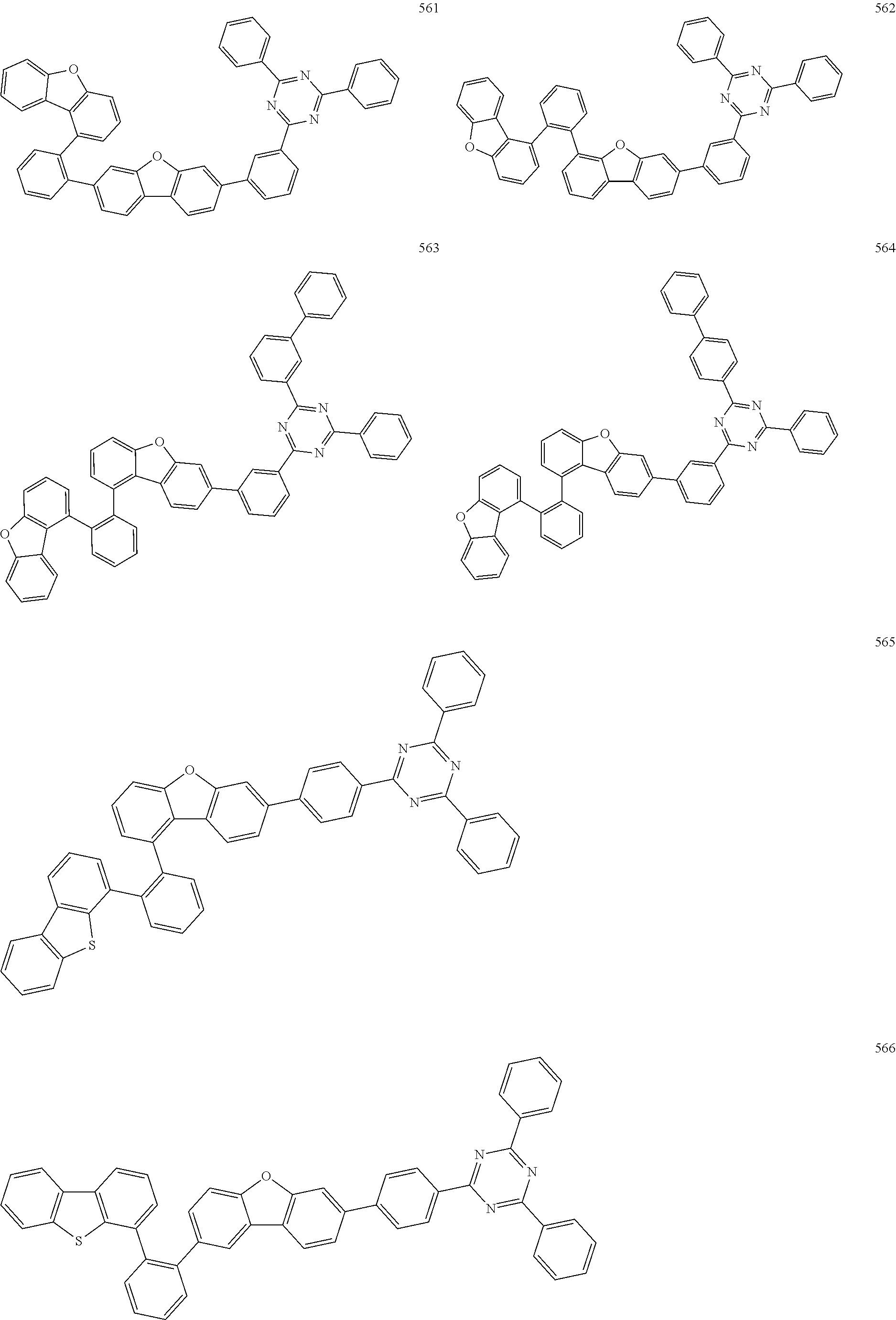

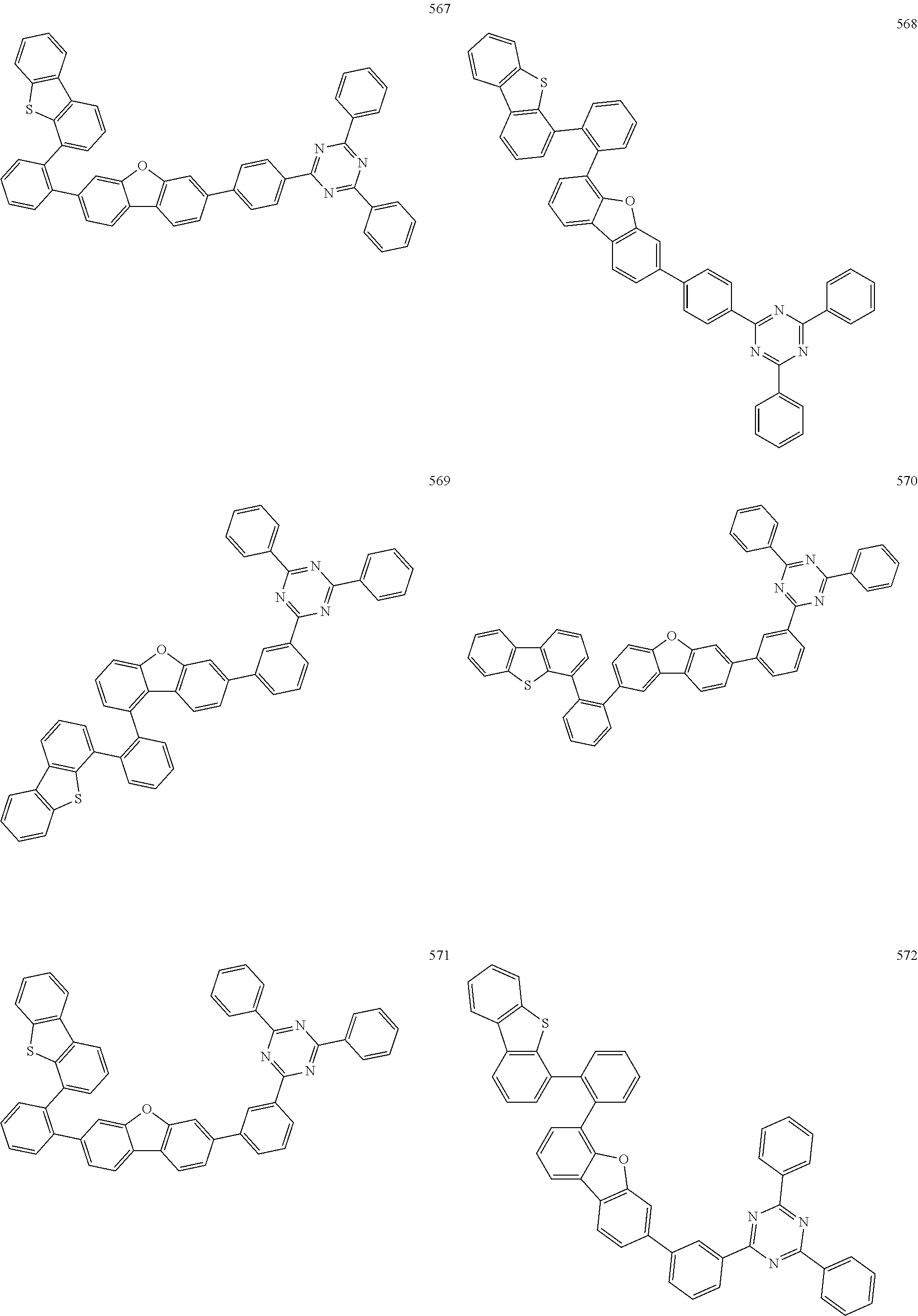

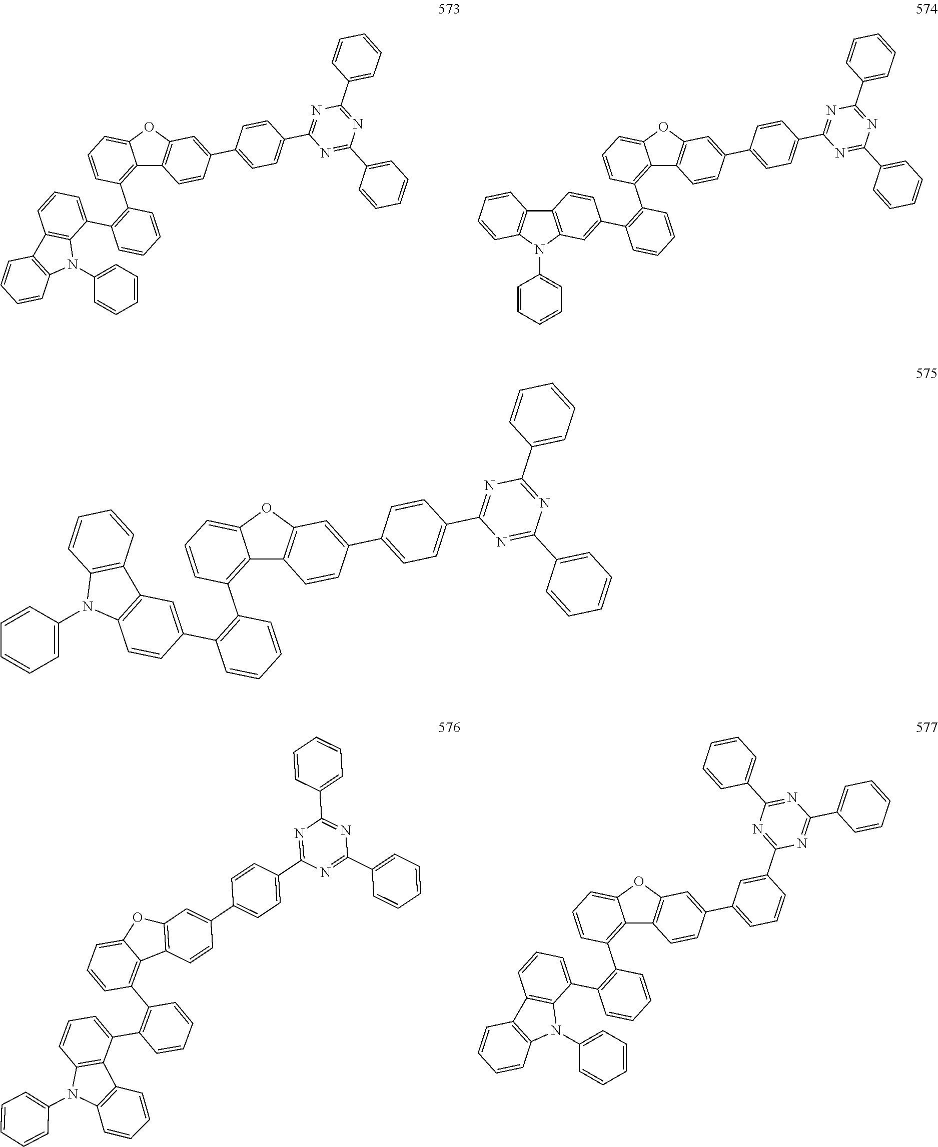

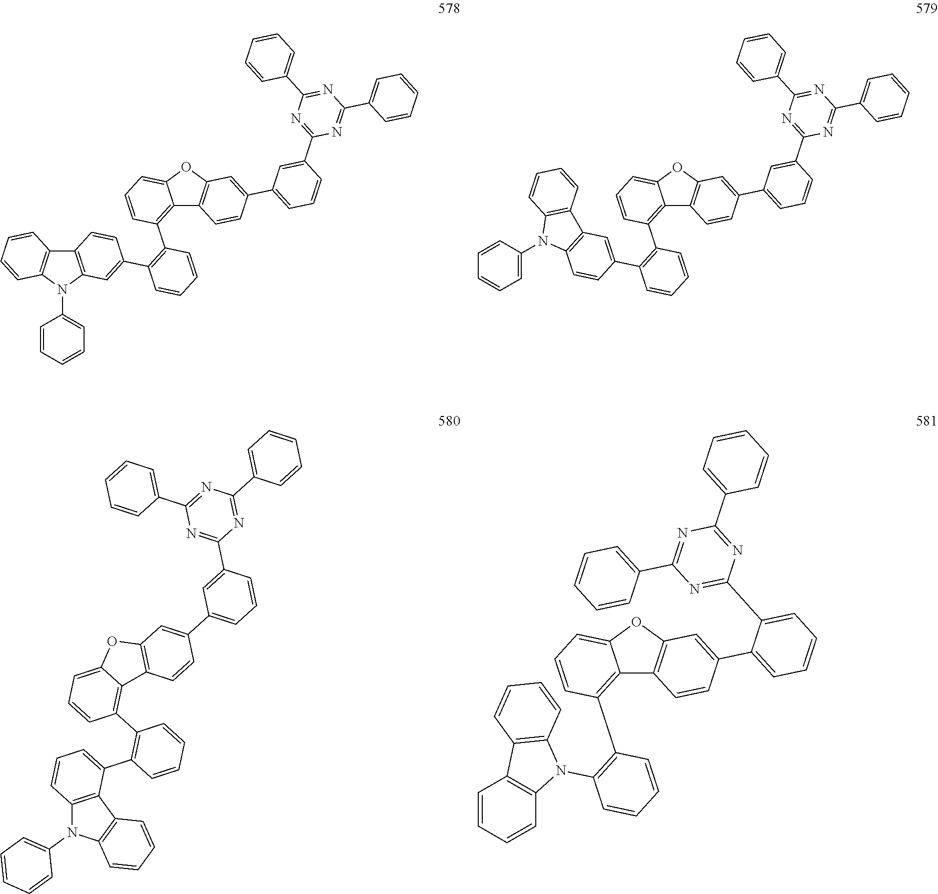 [SMILES: c1ccc(-c2nc(-c3ccccc3)nc(-c3cccc(-c4ccc5c(c4)oc4cccc(-c6ccccc6-c6ccc7c8ccccc8n(-c8ccccc8)c7c6)c45)c3)n2)cc1 ; c1ccc(-c2nc(-c3ccccc3)nc(-c3cccc(-c4ccc5c(c4)oc4cccc(-c6ccccc6-c6ccc7c(c6)c6ccccc6n7-c6ccccc6)c45)c3)n2)cc1 ; c1ccc(-c2nc(-c3ccccc3)nc(-c3cccc(-c4ccc5c(c4)oc4cccc(-c6ccccc6-c6cccc7c6c6ccccc6n7-c6ccccc6)c45)c3)n2)cc1 ; c1ccc(-c2nc(-c3ccccc3)nc(-c3ccccc3-c3ccc4c(c3)oc3cccc(-c5ccccc5-n5c6ccccc6c6ccccc65)c34)n2)cc1]

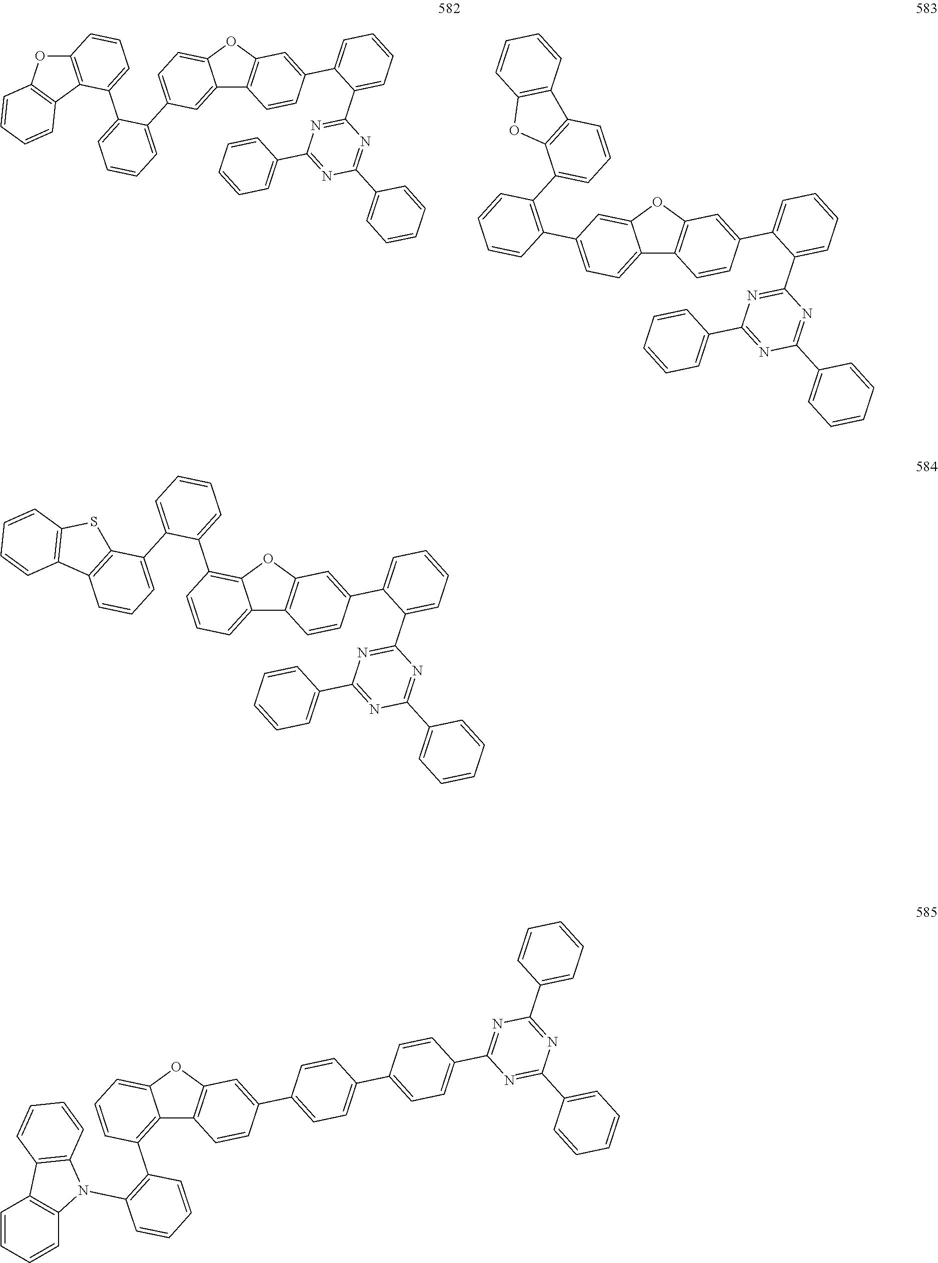 [SMILES: c1ccc(-c2nc(-c3ccccc3)nc(-c3ccccc3-c3ccc4c(c3)oc3ccc(-c5ccccc5-c5cccc6oc7ccccc7c56)cc34)n2)cc1 ; c1ccc(-c2nc(-c3ccccc3)nc(-c3ccccc3-c3ccc4c(c3)oc3cc(-c5ccccc5-c5cccc6c5oc5ccccc56)ccc34)n2)cc1 ; c1ccc(-c2nc(-c3ccccc3)nc(-c3ccccc3-c3ccc4c(c3)oc3c(-c5ccccc5-c5cccc6c5sc5ccccc56)cccc34)n2)cc1 ; c1ccc(-c2nc(-c3ccccc3)nc(-c3ccc(-c4ccc(-c5ccc6c(c5)oc5cccc(-c7ccccc7-n7c8ccccc8c8ccccc87)c56)cc4)cc3)n2)cc1]

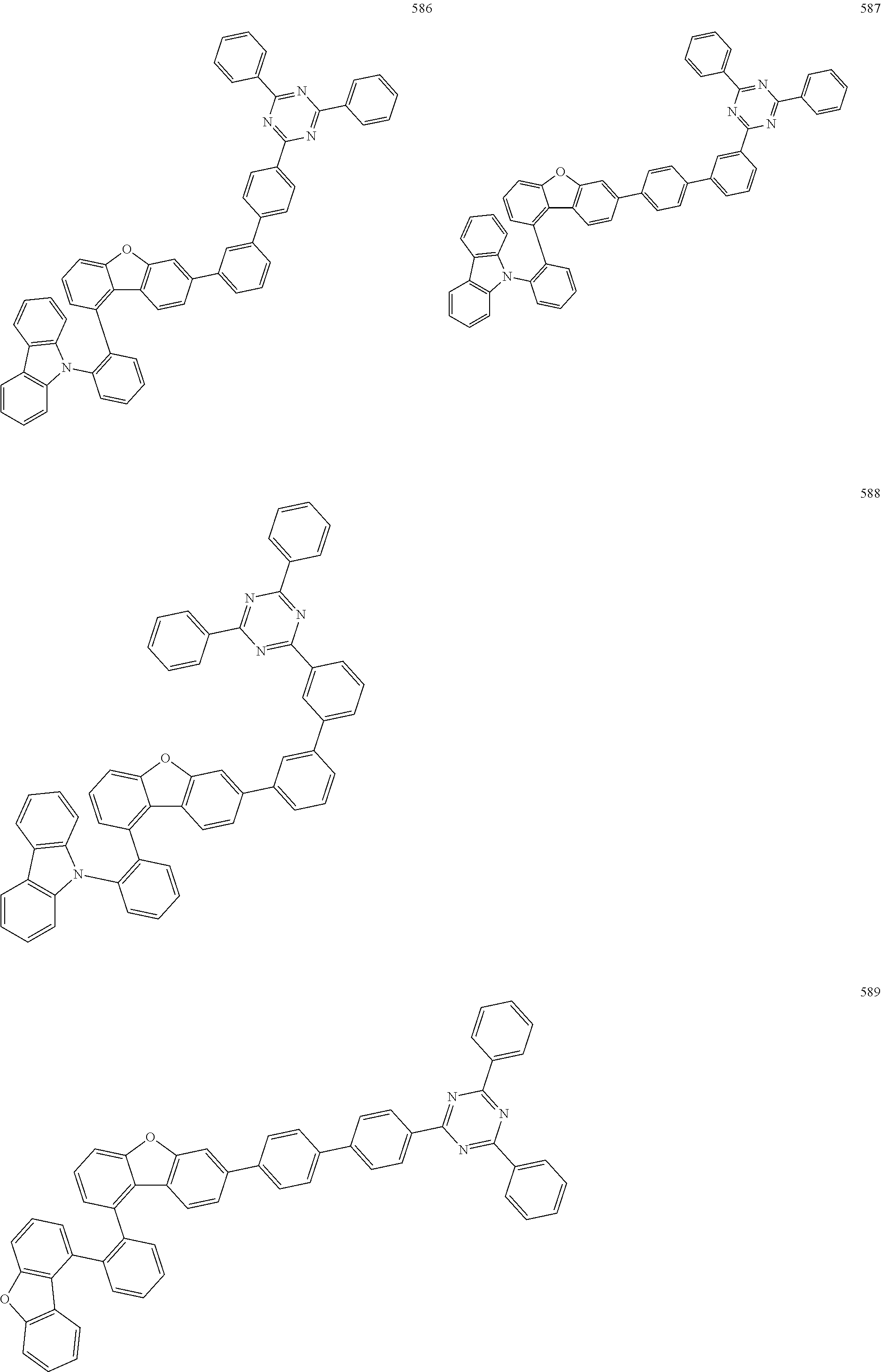 [SMILES: c1ccc(-c2nc(-c3ccccc3)nc(-c3ccc(-c4cccc(-c5ccc6c(c5)oc5cccc(-c7ccccc7-n7c8ccccc8c8ccccc87)c56)c4)cc3)n2)cc1 ; c1ccc(-c2nc(-c3ccccc3)nc(-c3cccc(-c4ccc(-c5ccc6c(c5)oc5cccc(-c7ccccc7-n7c8ccccc8c8ccccc87)c56)cc4)c3)n2)cc1 ; c1ccc(-c2nc(-c3ccccc3)nc(-c3cccc(-c4cccc(-c5ccc6c(c5)oc5cccc(-c7ccccc7-n7c8ccccc8c8ccccc87)c56)c4)c3)n2)cc1 ; c1ccc(-c2nc(-c3ccccc3)nc(-c3ccc(-c4ccc(-c5ccc6c(c5)oc5cccc(-c7ccccc7-c7cccc8oc9ccccc9c78)c56)cc4)cc3)n2)cc1]

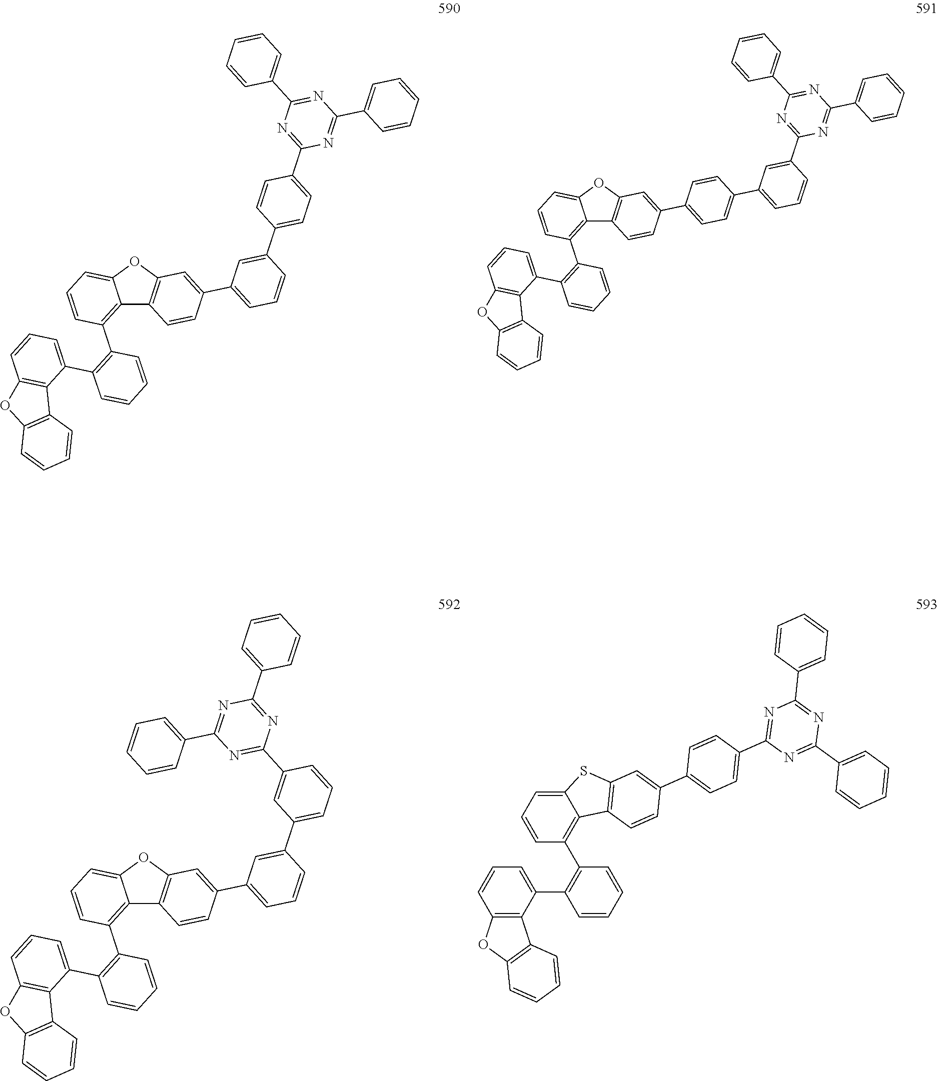 [SMILES: c1ccc(-c2nc(-c3ccccc3)nc(-c3ccc(-c4cccc(-c5ccc6c(c5)oc5cccc(-c7ccccc7-c7cccc8oc9ccccc9c78)c56)c4)cc3)n2)cc1 ; c1ccc(-c2nc(-c3ccccc3)nc(-c3cccc(-c4ccc(-c5ccc6c(c5)oc5cccc(-c7ccccc7-c7cccc8oc9ccccc9c78)c56)cc4)c3)n2)cc1 ; c1ccc(-c2nc(-c3ccccc3)nc(-c3cccc(-c4cccc(-c5ccc6c(c5)oc5cccc(-c7ccccc7-c7cccc8oc9ccccc9c78)c56)c4)c3)n2)cc1 ; c1ccc(-c2nc(-c3ccccc3)nc(-c3ccc(-c4ccc5c(c4)sc4cccc(-c6ccccc6-c6cccc7oc8ccccc8c67)c45)cc3)n2)cc1]

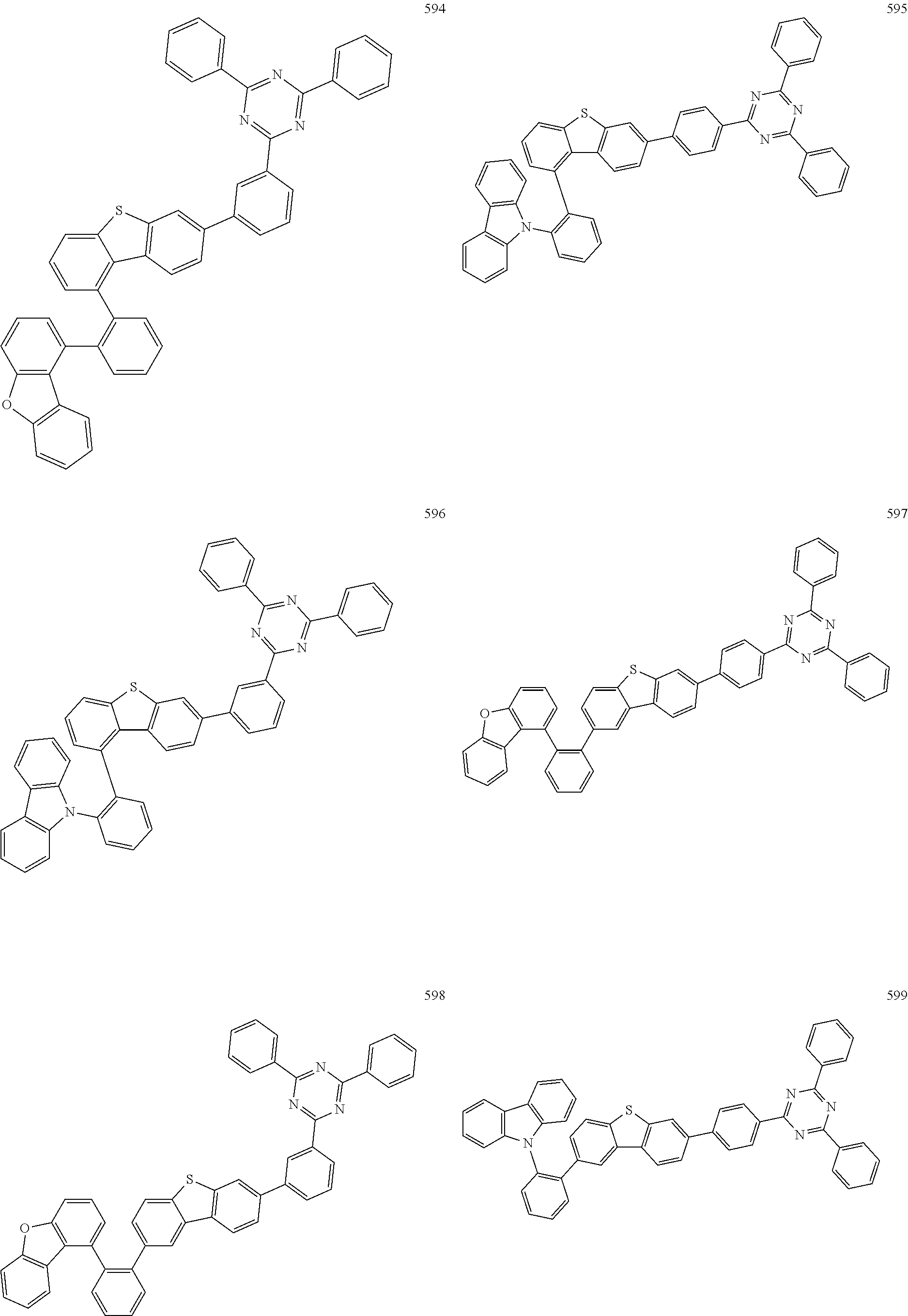

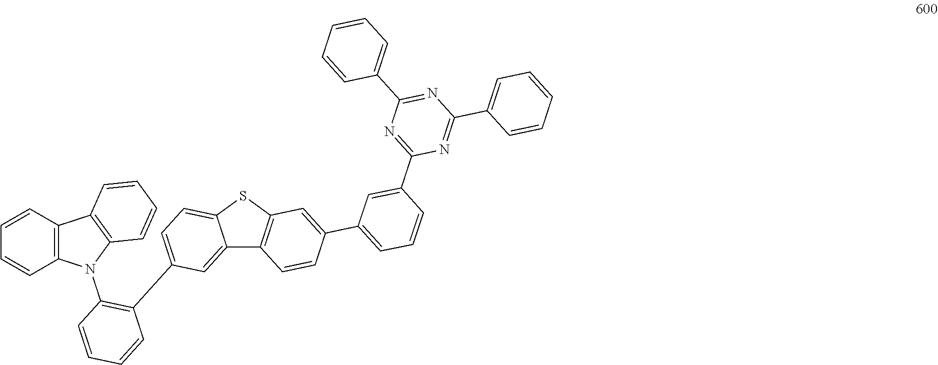 [SMILES: c1ccc(-c2nc(-c3ccccc3)nc(-c3cccc(-c4ccc5c(c4)sc4ccc(-c6ccccc6-n6c7ccccc7c7ccccc76)cc45)c3)n2)cc1]

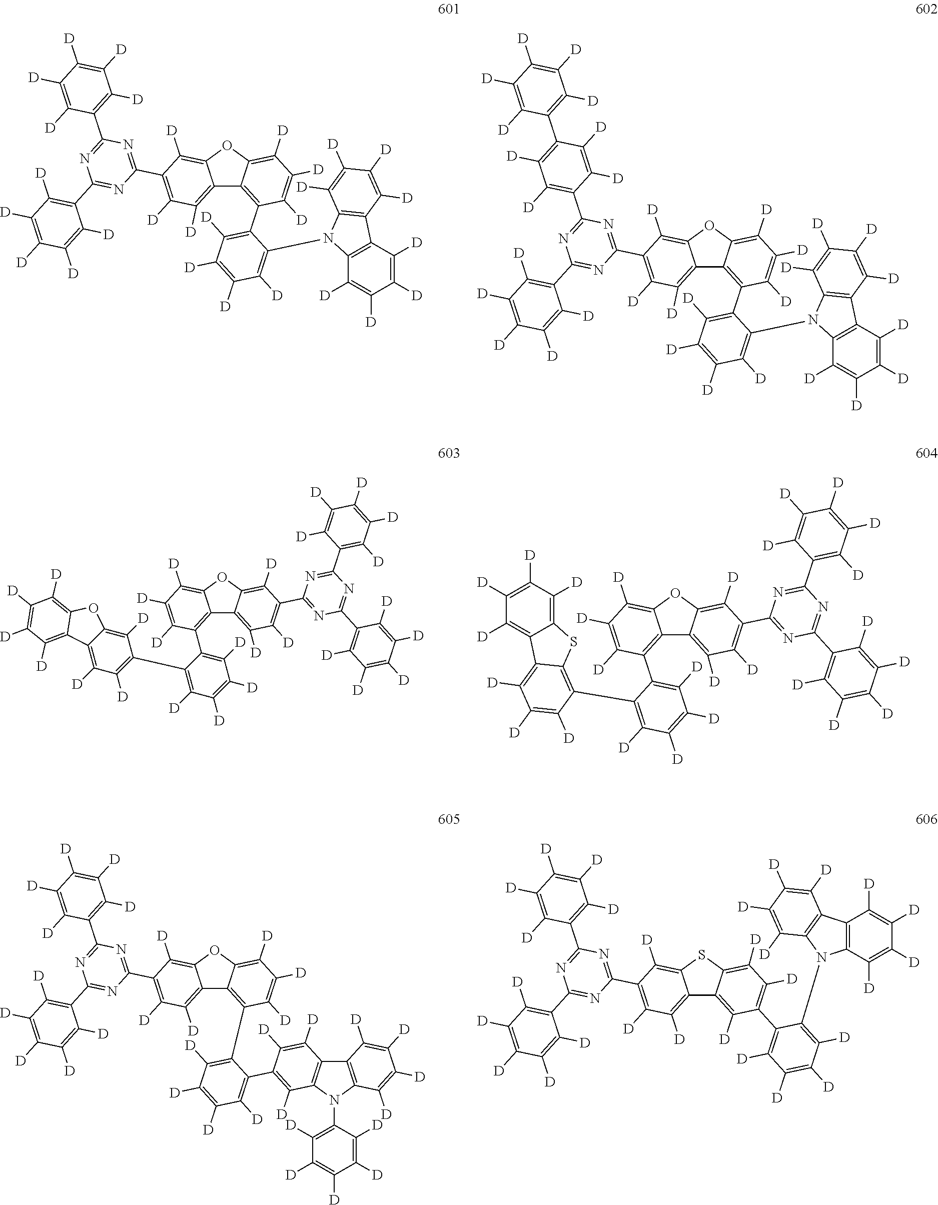

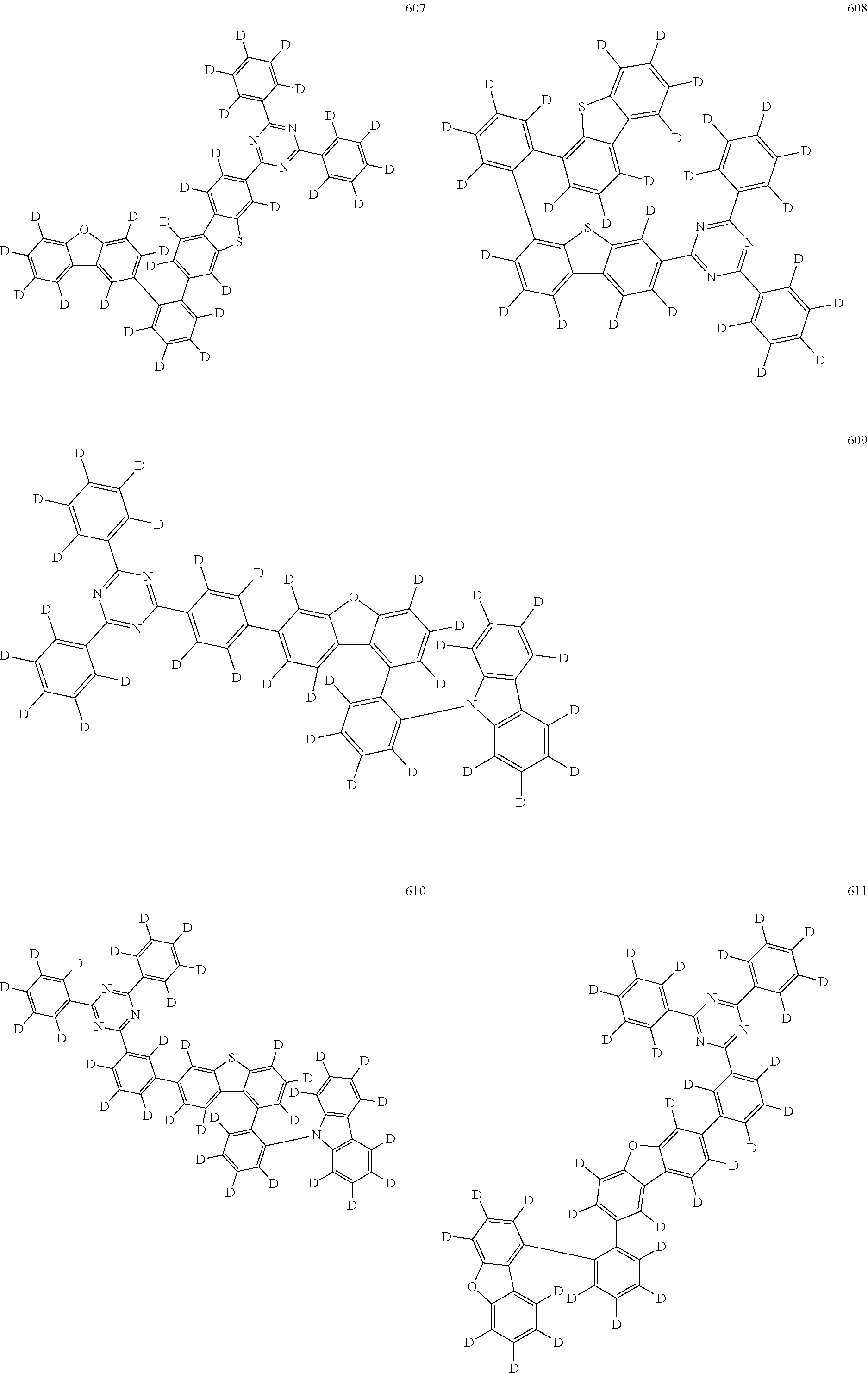

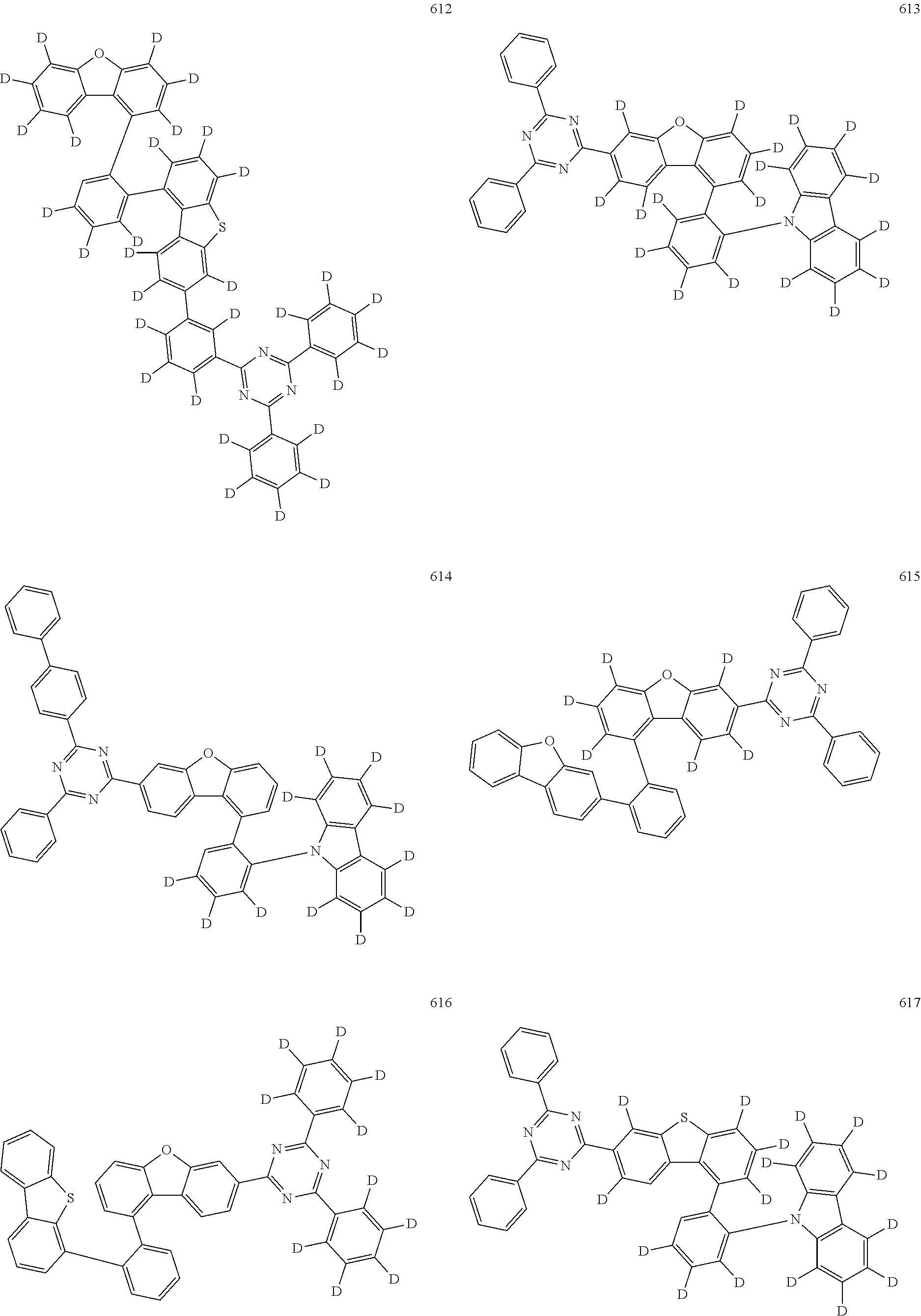

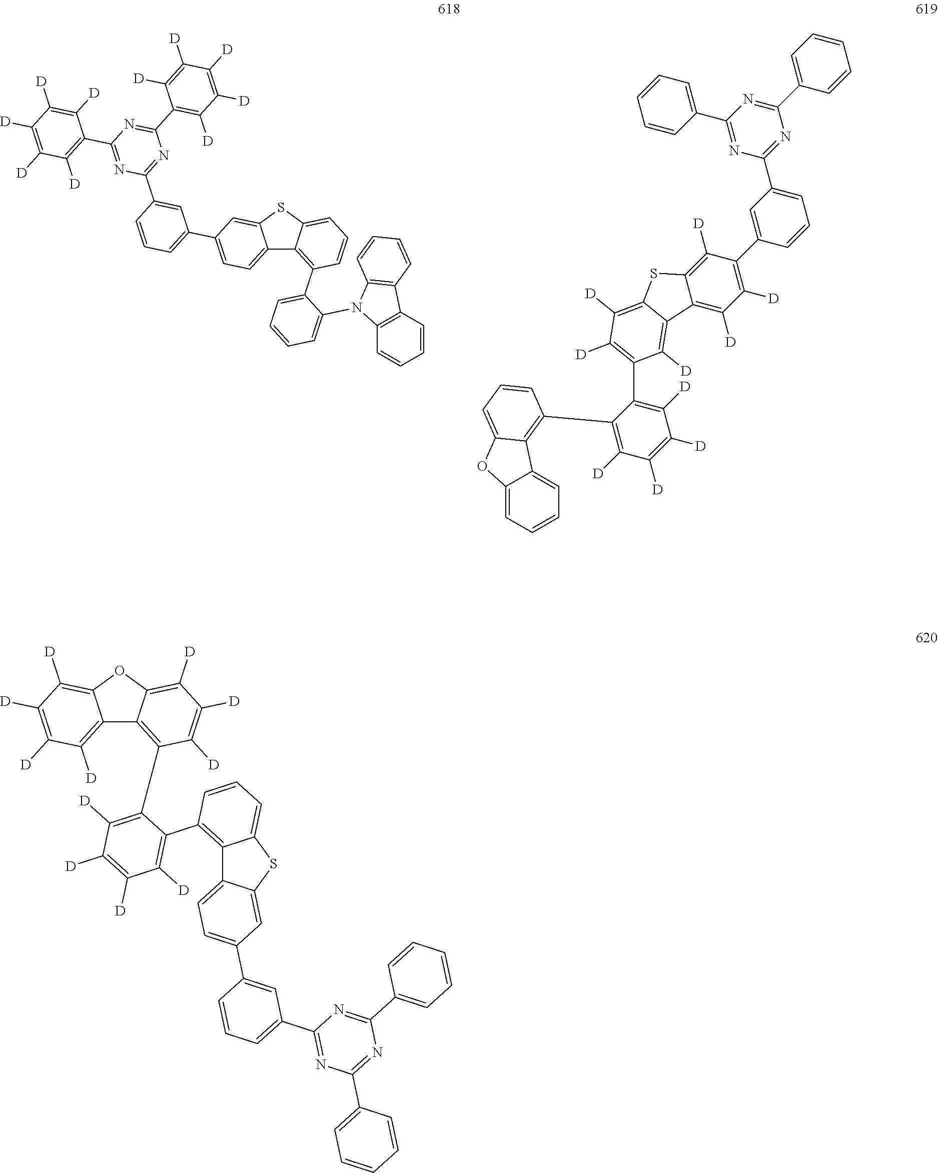 [SMILES: [2H]c1c([2H])c([2H])c(-c2nc(-c3cccc(-c4ccc5c(c4)sc4cccc(-c6ccccc6-n6c7ccccc7c7ccccc76)c45)c3)nc(-c3c([2H])c([2H])c([2H])c([2H])c3[2H])n2)c([2H])c1[2H] ; [2H]c1c([2H])c([2H])c(-c2cccc3oc4ccccc4c23)c(-c2c([2H])c([2H])c3sc4c([2H])c(-c5cccc(-c6nc(-c7ccccc7)nc(-c7ccccc7)n6)c5)c([2H])c([2H])c4c3c2[2H])c1[2H] ; [2H]c1c([2H])c([2H])c(-c2c([2H])c([2H])c([2H])c3oc4c([2H])c([2H])c([2H])c([2H])c4c23)c(-c2cccc3sc4cc(-c5cccc(-c6nc(-c7ccccc7)nc(-c7ccccc7)n6)c5)ccc4c23)c1[2H]]

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a hole transport layer material, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

In another exemplary embodiment of the present specification, provided is an organic light emitting device including: a first electrode; a second electrode; and an organic material layer having one or more layers provided between the first electrode and the second electrode, in which one or more layers of the organic material layer include the one or more heterocyclic compounds of Chemical Formula 1.

In an exemplary embodiment of the present specification, the organic material layer includes a light emitting layer, and the light emitting layer may include one or more of the heterocyclic compounds.

In an exemplary embodiment of the present specification, the organic material layer includes a light emitting layer, and the light emitting layer may include one of the heterocyclic compounds.

In an exemplary embodiment of the present specification, the organic material layer includes a light emitting layer, the light emitting layer includes a host, and the host may include one or more of the heterocyclic compounds.

In an exemplary embodiment of the present specification, the organic material layer includes a light emitting layer, the light emitting layer includes a host, and the host may include one of the heterocyclic compounds.

In an exemplary embodiment of the present specification, the organic material layer includes a light emitting layer, the light emitting layer includes a host, the host includes a green host, and the green host may include one or more of the heterocyclic compounds.

In an exemplary embodiment of the present specification, the organic material layer includes a light emitting layer, the light emitting layer includes a host, the host includes a red host, and the red host may include one or more of the heterocyclic compounds.

In an exemplary embodiment of the present specification, the organic material layer includes a light emitting layer, the light emitting layer includes a host, the host includes a blue host, and the blue host may include one or more of the heterocyclic compounds.

In an exemplary embodiment of the present specification, the organic material layer includes a light emitting layer, and the light emitting layer may include the heterocyclic compound as an N-type host.

In an exemplary embodiment of the present specification, the organic material layer including the heterocyclic compound may further include a compound of the following Chemical Formula 2.

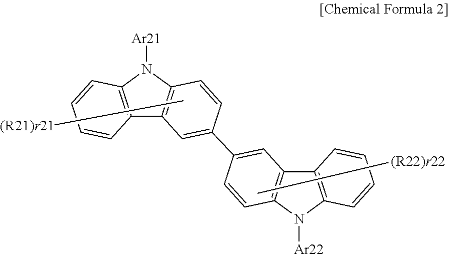

In Chemical Formula 2,
Ar21 and Ar22 are each independently a substituted or unsubstituted C6 to C60 aryl group; or a substituted or unsubstituted C2 to C60 heteroaryl group,
R21 and R22 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
r21 and r22 are each an integer from 0 to 7, and when r21 and r22 are each 2 or higher, substituents in the parenthesis are the same as or different from each other.

In an exemplary embodiment of the present specification, the organic material layer including the heterocyclic compound may further include the compound of Chemical Formula 2 as a P-type host.

In an exemplary embodiment of the present specification, Ar21 and Ar22 are each independently a substituted or unsubstituted C6 to C60 aryl group; or a substituted or unsubstituted C2 to C60 heteroaryl group.

In an exemplary embodiment of the present specification, Ar21 and Ar22 may be each independently a substituted or unsubstituted C6 to C30 aryl group; or a substituted or unsubstituted C2 to C30 heteroaryl group.

In an exemplary embodiment of the present specification, Ar21 and Ar22 may be each independently a substituted or unsubstituted phenyl group; a substituted or unsubstituted biphenyl group; a substituted or unsubstituted terphenyl group; a substituted or unsubstituted triphenylene group; a substituted or unsubstituted fluorenyl group; a substituted or unsubstituted dibenzofuran group; or substituted or unsubstituted dibenzothiophene group.

In an exemplary embodiment of the present specification, Ar21 and Ar22 may be a C6 to C30 aryl group unsubstituted or substituted with one or more substituents of deuterium and an alkyl group; or a C2 to C30 heteroaryl group unsubstituted or substituted with deuterium.

In an exemplary embodiment of the present specification, Ar21 and Ar22 may be each independently a phenyl group unsubstituted or substituted with deuterium; a biphenyl group unsubstituted or substituted with deuterium; a terphenyl group unsubstituted or substituted with deuterium; a triphenylene group unsubstituted or substituted with deuterium; a fluorenyl group unsubstituted or substituted with one or more substituents of deuterium and an alkyl group; a dibenzofuran group unsubstituted or substituted with deuterium; or a dibenzothiophene group unsubstituted or substituted with deuterium.

In an exemplary embodiment of the present specification, R21 and R22 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R21 and R22 may be each independently hydrogen; deuterium; a halogen group; a cyano group; a substituted or unsubstituted C1 to C30 alkyl group; a substituted or unsubstituted C3 to C30 cycloalkyl group; a substituted or unsubstituted C2 to C30 heterocycloalkyl group; a substituted or unsubstituted C6 to C30 aryl group; or a substituted or unsubstituted C2 to C30 heteroaryl group.

In an exemplary embodiment of the present specification, R21 and R22 may be each independently hydrogen; deuterium; a halogen group; a cyano group; a substituted or unsubstituted C1 to C30 alkyl group; a substituted or unsubstituted C6 to C30 aryl group; or a substituted or unsubstituted C2 to C30 heteroaryl group.

In an exemplary embodiment of the present specification, R21 and R22 may be each independently hydrogen; deuterium; a substituted or unsubstituted C6 to C30 aryl group; or a substituted or unsubstituted C2 to C30 heteroaryl group.

In an exemplary embodiment of the present specification, R21 and R22 may be each independently hydrogen; or deuterium.

In an exemplary embodiment of the present specification, the deuterium content of Chemical Formula 2 may be 0% to 100%.

In an exemplary embodiment of the present specification, the deuterium content of Chemical Formula 2 may be 0%.

In an exemplary embodiment of the present specification, the deuterium content of Chemical Formula 2 may be 20% to 100%.

In an exemplary embodiment of the present specification, the deuterium content of Chemical Formula 2 may be 30% to 100%.

In an exemplary embodiment of the present specification, the deuterium content of Chemical Formula 2 may be 50% to 100%.

In an exemplary embodiment of the present specification, the deuterium content of Chemical Formula 2 may be 100%.

In an exemplary embodiment of the present specification, the deuterium content of Chemical Formula 2 may be 0% or 20% to 100%.

In an exemplary embodiment of the present specification, the deuterium content of Chemical Formula 2 may be 0% or 30% to 100%.

In an exemplary embodiment of the present specification, the deuterium content of Chemical Formula 2 may be 0% or 50% to 100%.

In an exemplary embodiment of the present specification, the compound of Chemical Formula 2 satisfies the deuterium content in the above range, and in the case of a compound that includes deuterium, the intermolecular distance tends to be more narrowly packed when the compound is deposited into a thin film compared to a compound that does not include deuterium, and the compound is deposited on a more uniform surface without agglomeration. Furthermore, the compound including deuterium shows much more balanced charge transport characteristics than the compound that does not include deuterium, and the high single bond dissociation energy of carbon and deuterium increases the stability of the entire molecule, so that there is an effect that the service life of the device is improved.

In an exemplary embodiment of the present specification, Chemical Formula 2 may be represented by any one of the following compounds.

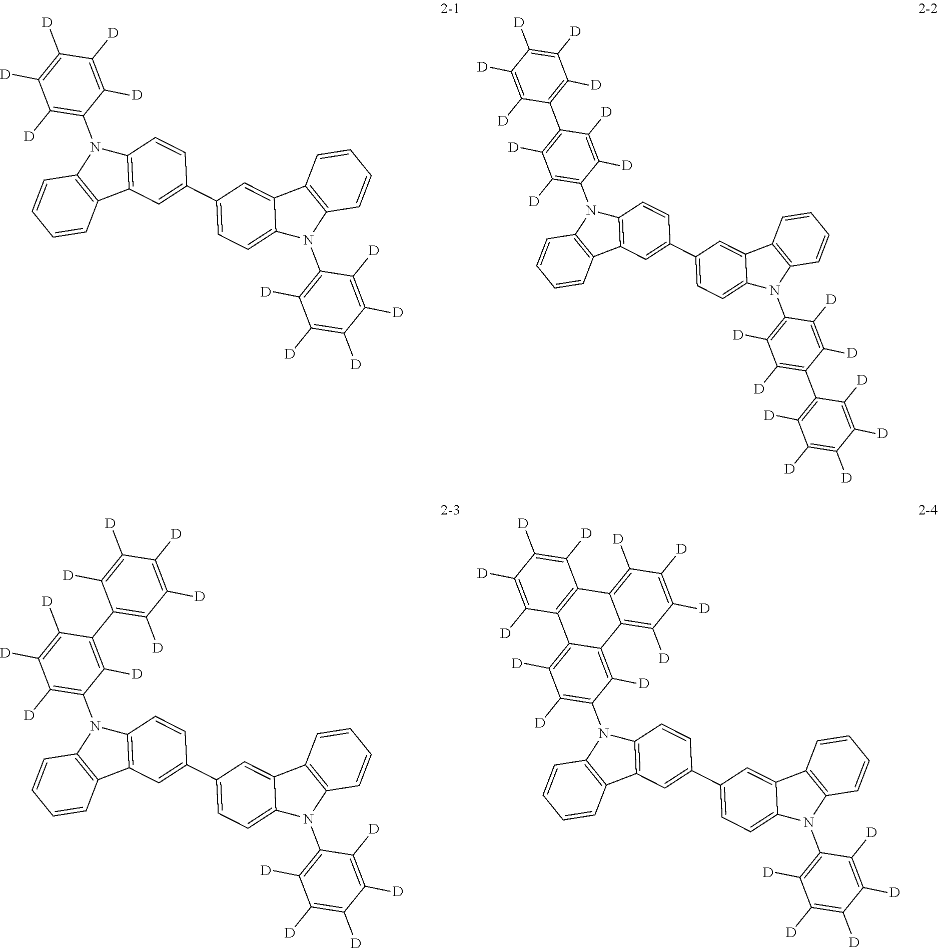

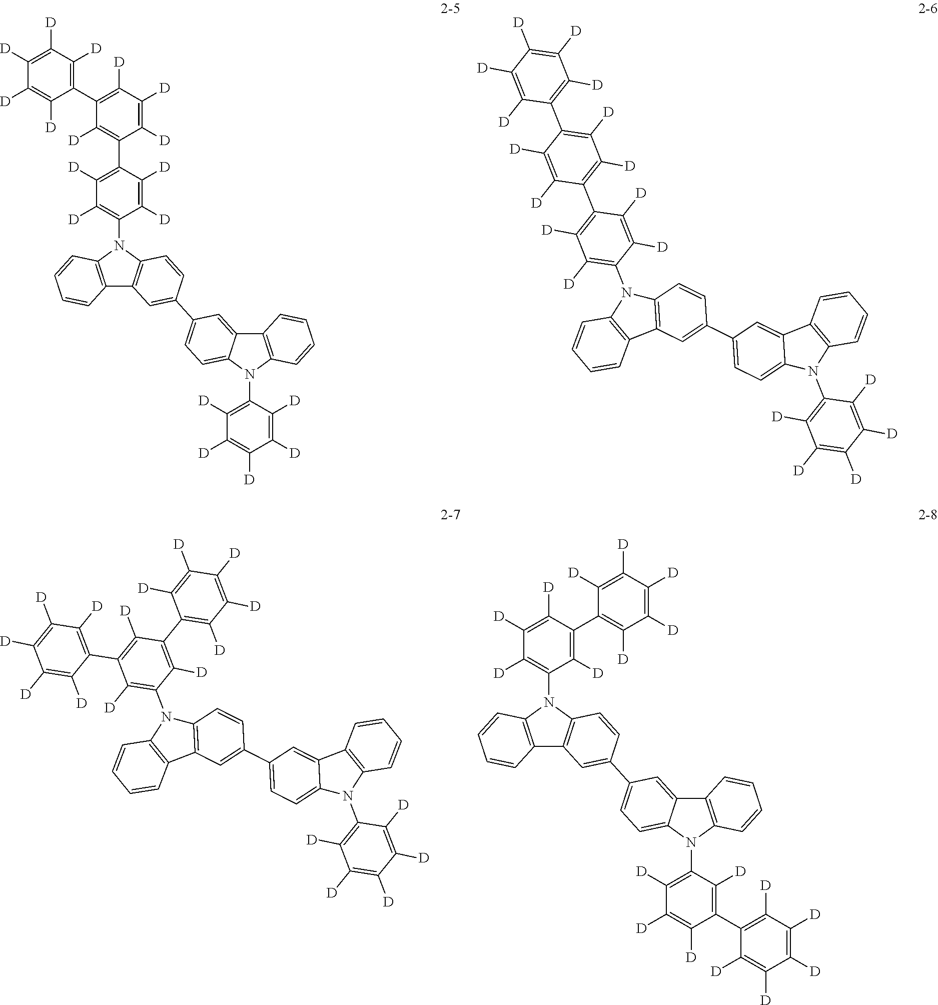

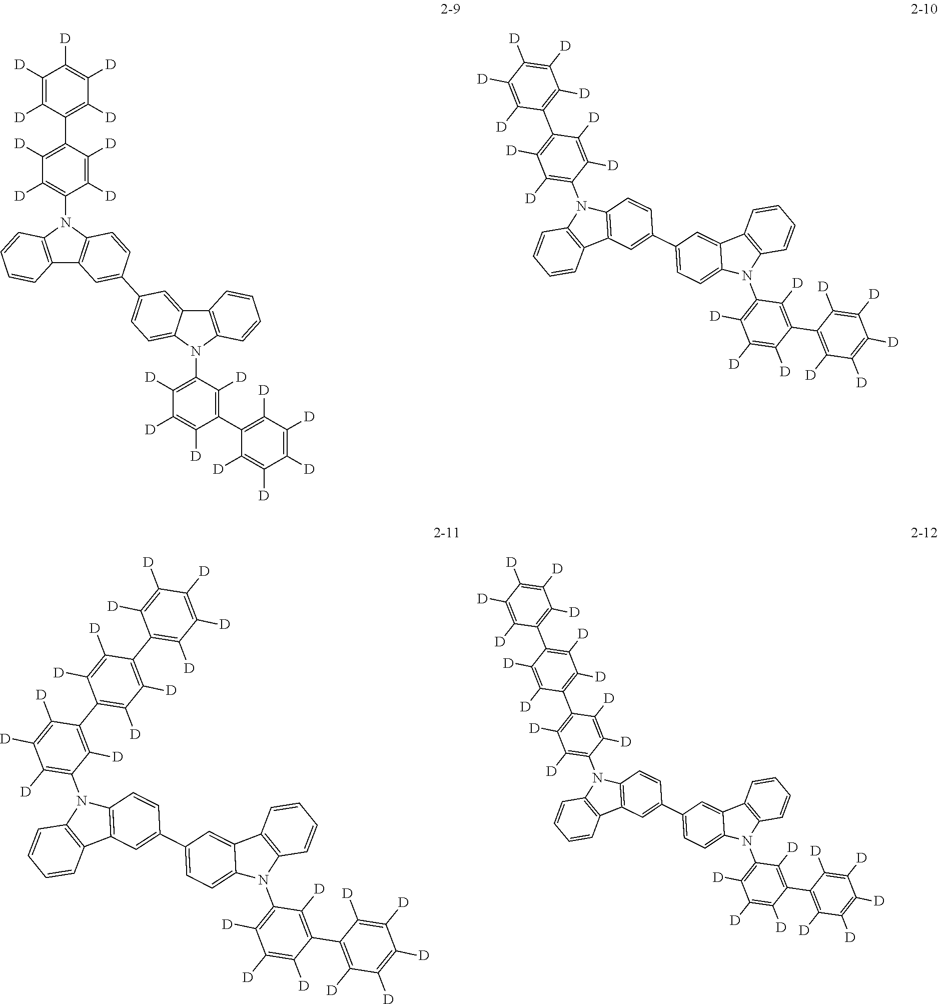

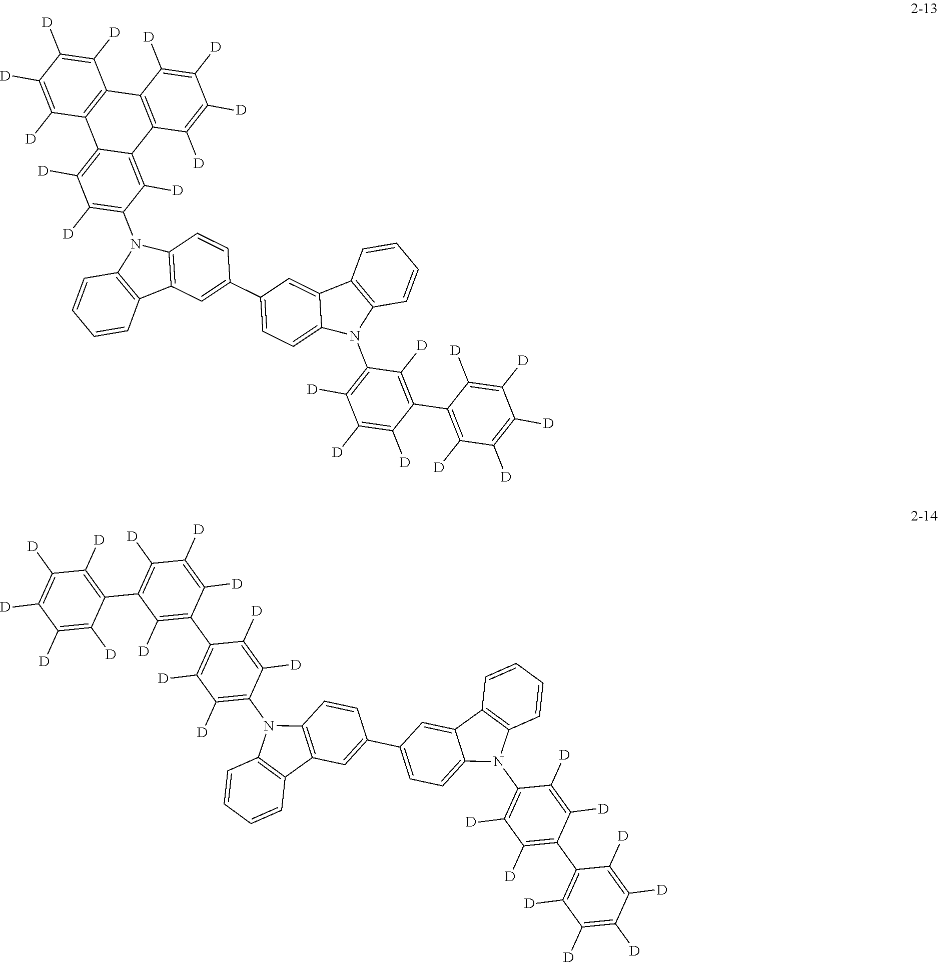 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c6c7c([2H])c([2H])c([2H])c([2H])c7c7c([2H])c([2H])c([2H])c([2H])c7c6c5[2H])ccc43)c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c(-c7c([2H])c([2H])c([2H])c([2H])c7[2H])c6[2H])c([2H])c5[2H])ccc43)c([2H])c2[2H])c([2H])c1[2H]]

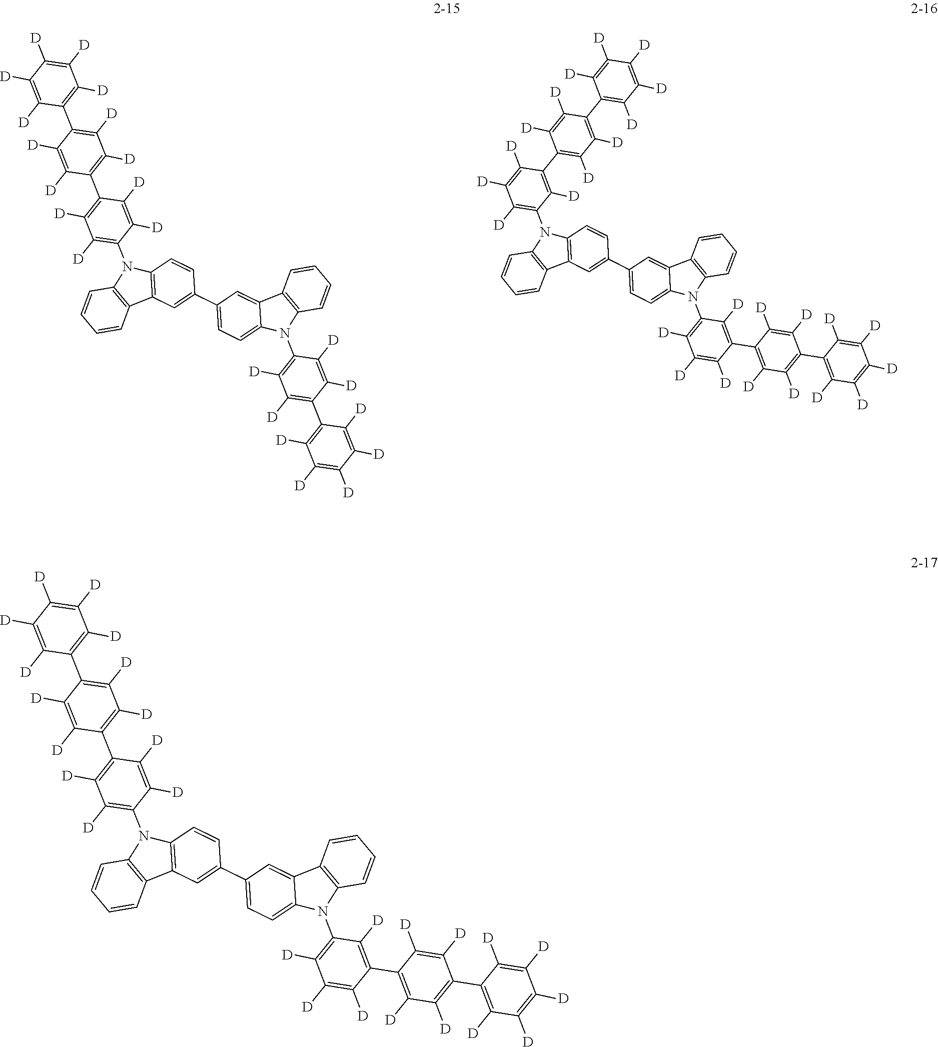

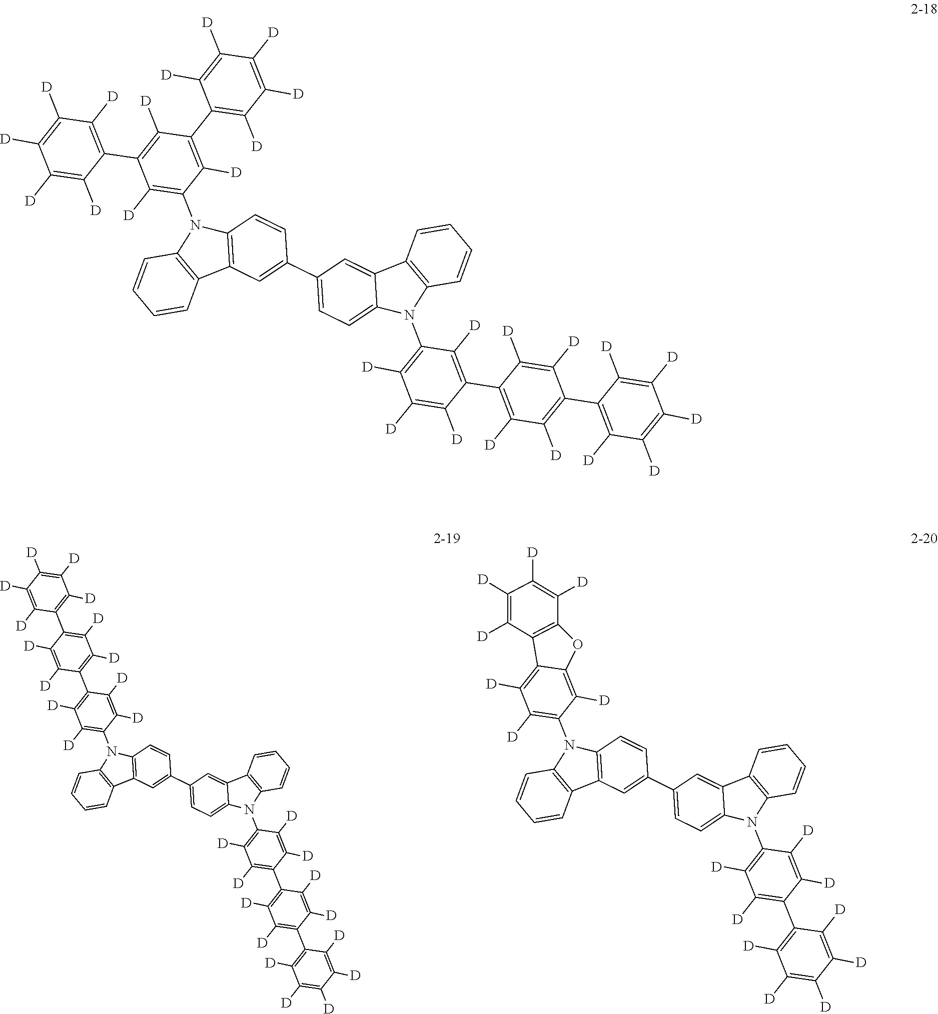

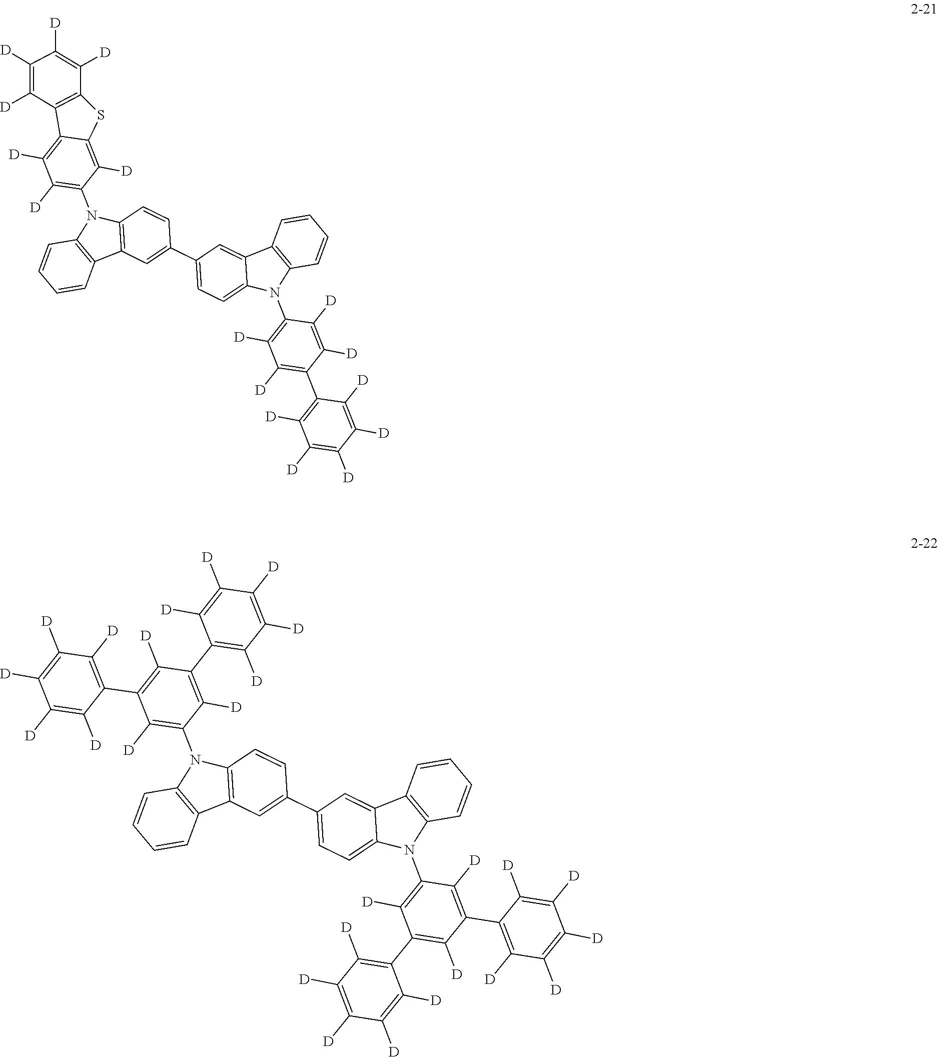 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c6c(sc7c([2H])c([2H])c([2H])c([2H])c76)c5[2H])ccc43)c([2H])c2[2H])c([2H])c1[2H] ; [2H]c1c([2H])c([2H])c(-c2c([2H])c(-c3c([2H])c([2H])c([2H])c([2H])c3[2H])c([2H])c(-n3c4ccccc4c4cc(-c5ccc6c(c5)c5ccccc5n6-c5c([2H])c(-c6c([2H])c([2H])c([2H])c([2H])c6[2H])c([2H])c(-c6c([2H])c([2H])c([2H])c([2H])c6[2H])c5[2H])ccc43)c2[2H])c([2H])c1[2H]]

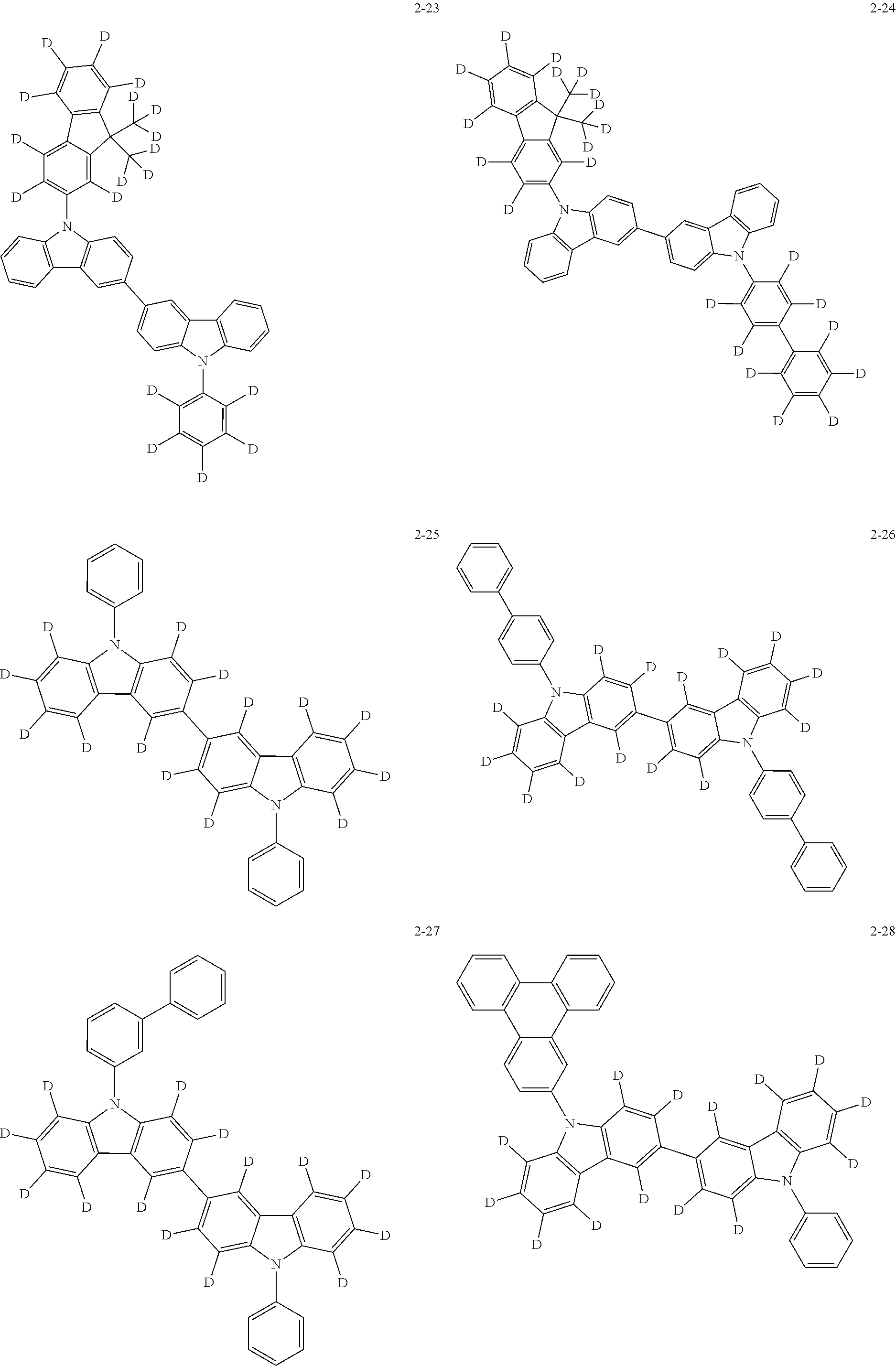

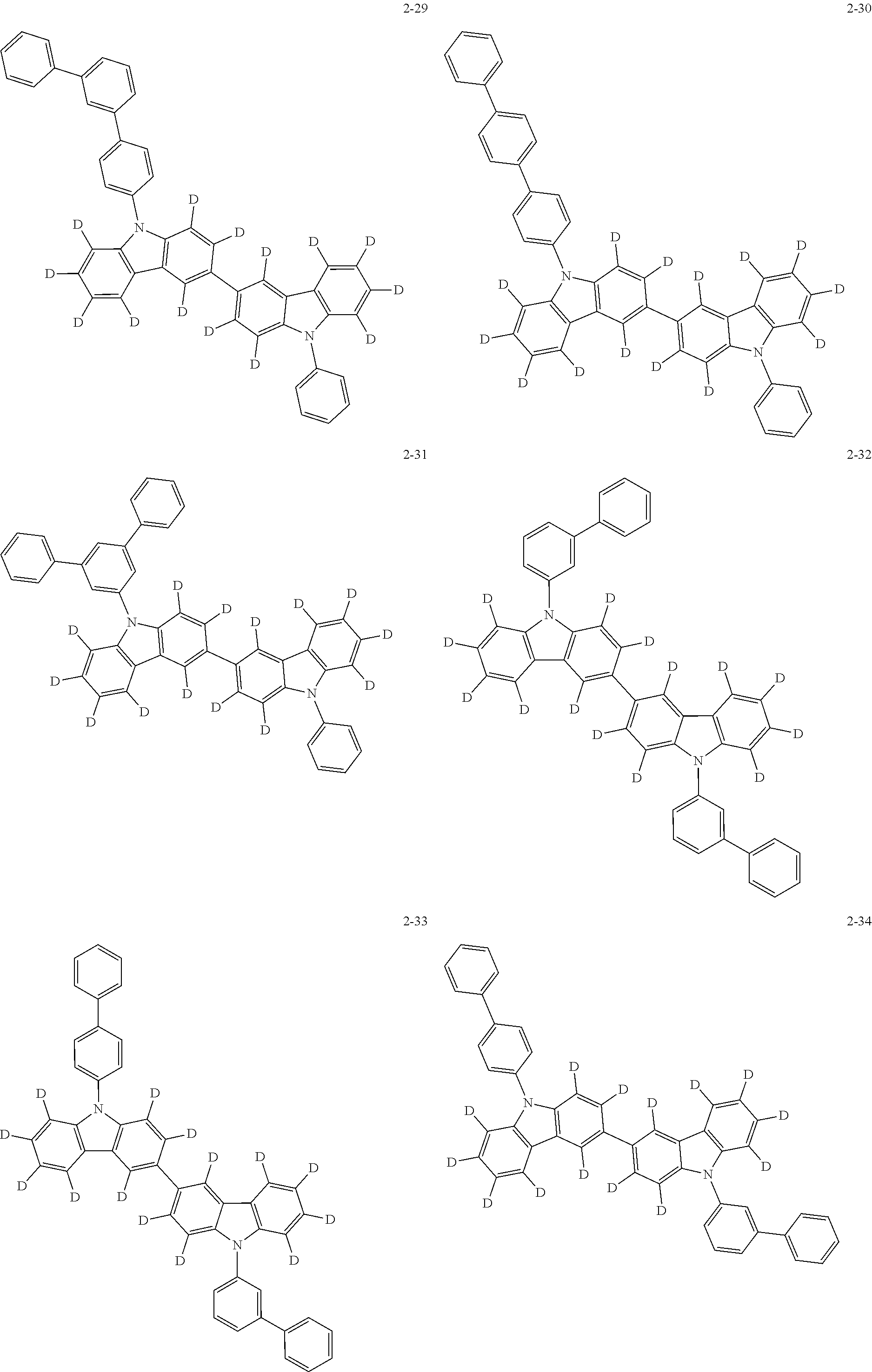

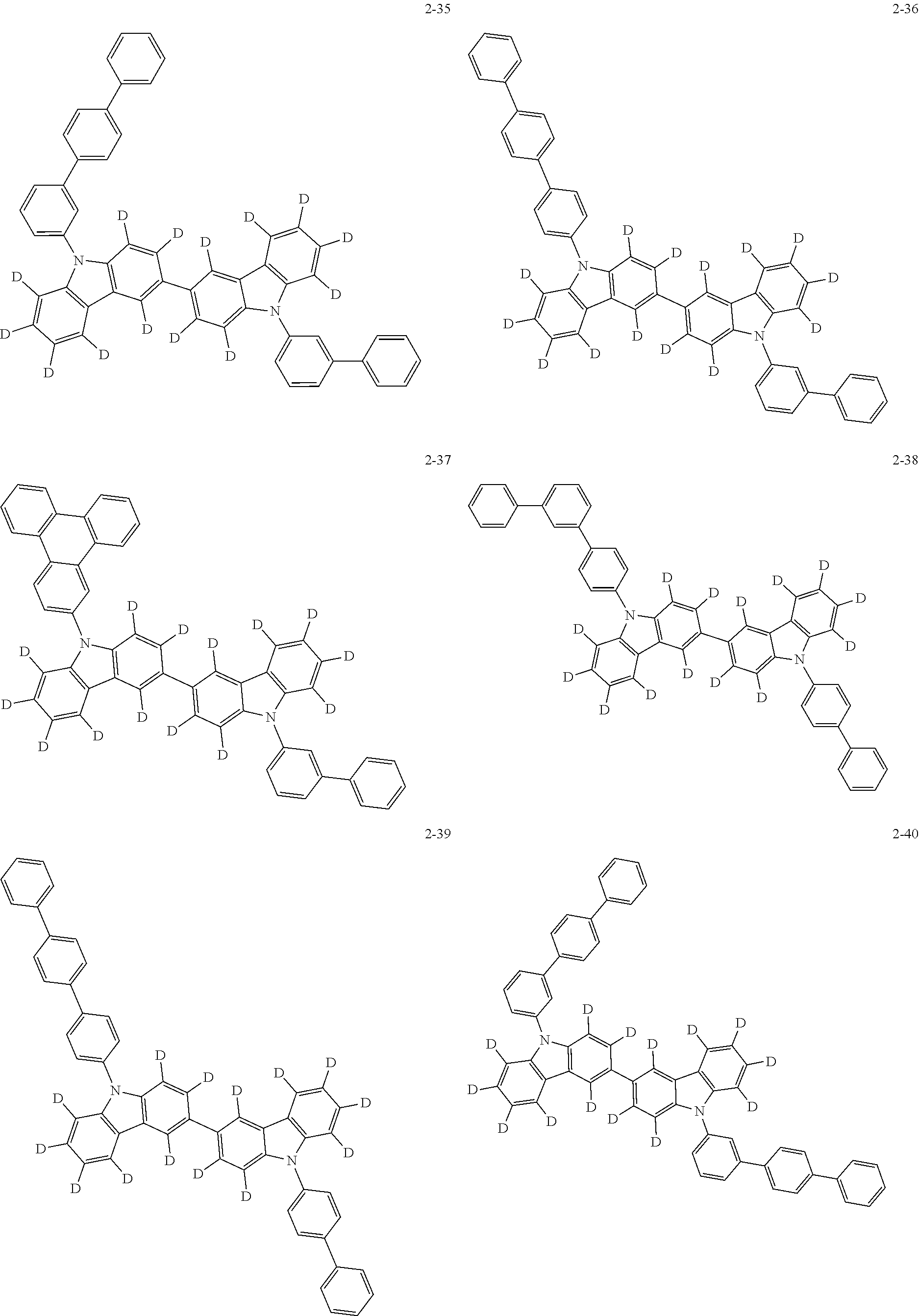

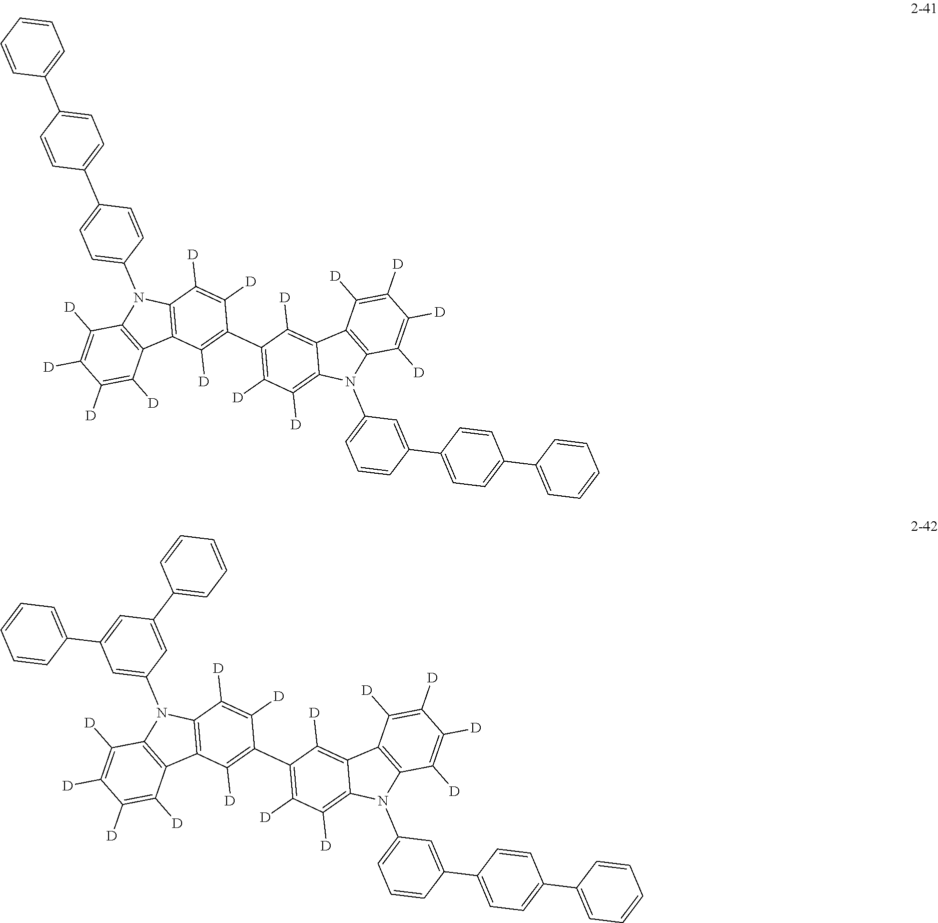 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1ccc(-c2ccc(-c3ccccc3)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1cccc(-c2ccc(-c3ccccc3)cc2)c1]

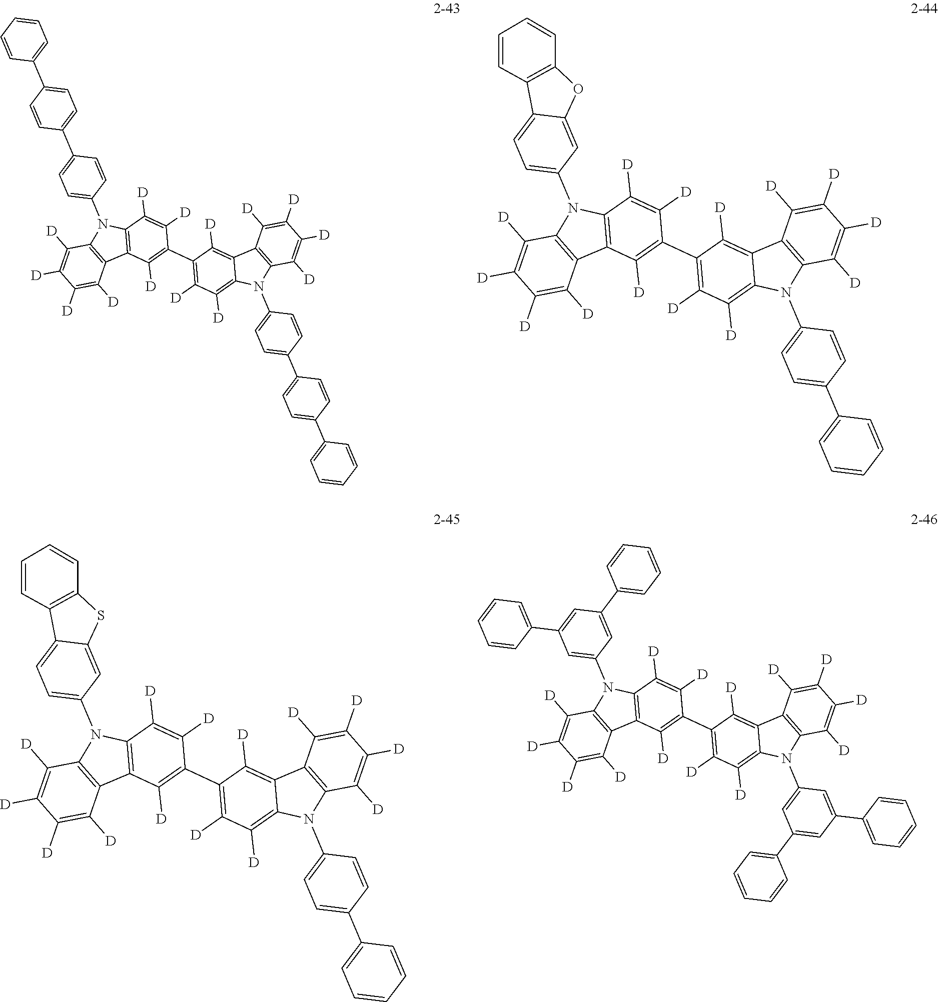

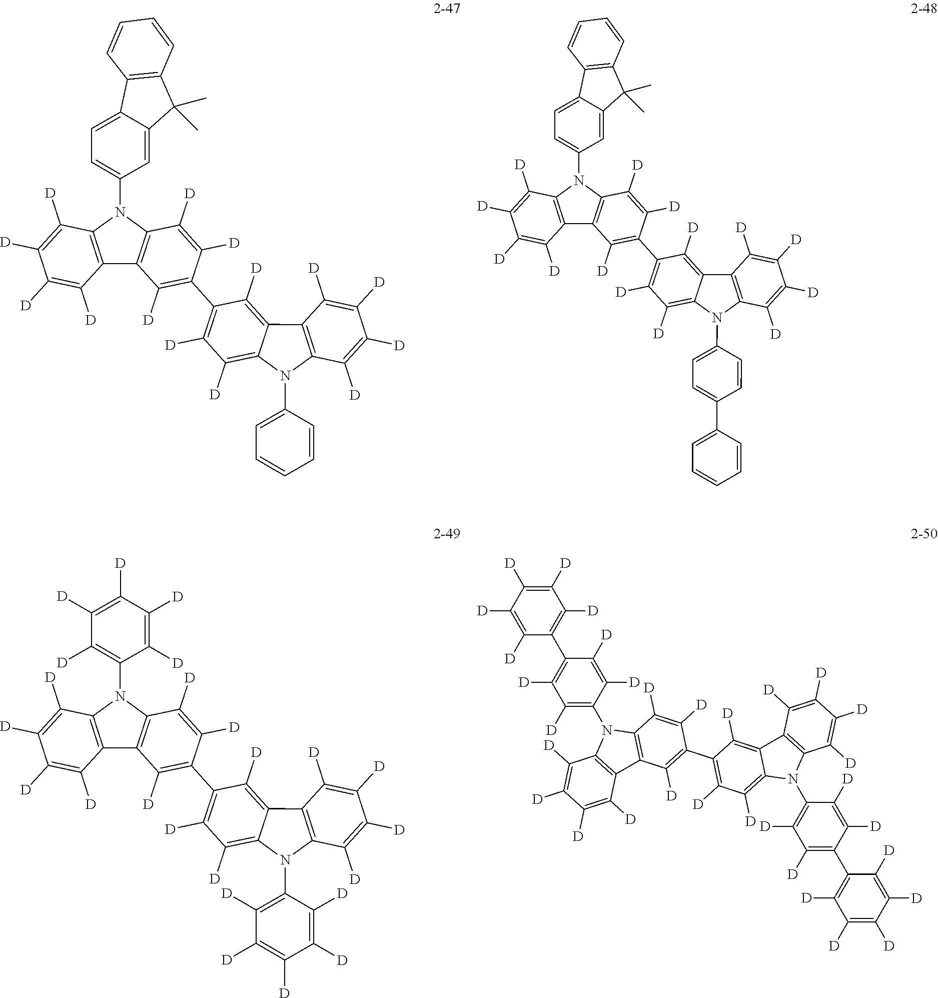

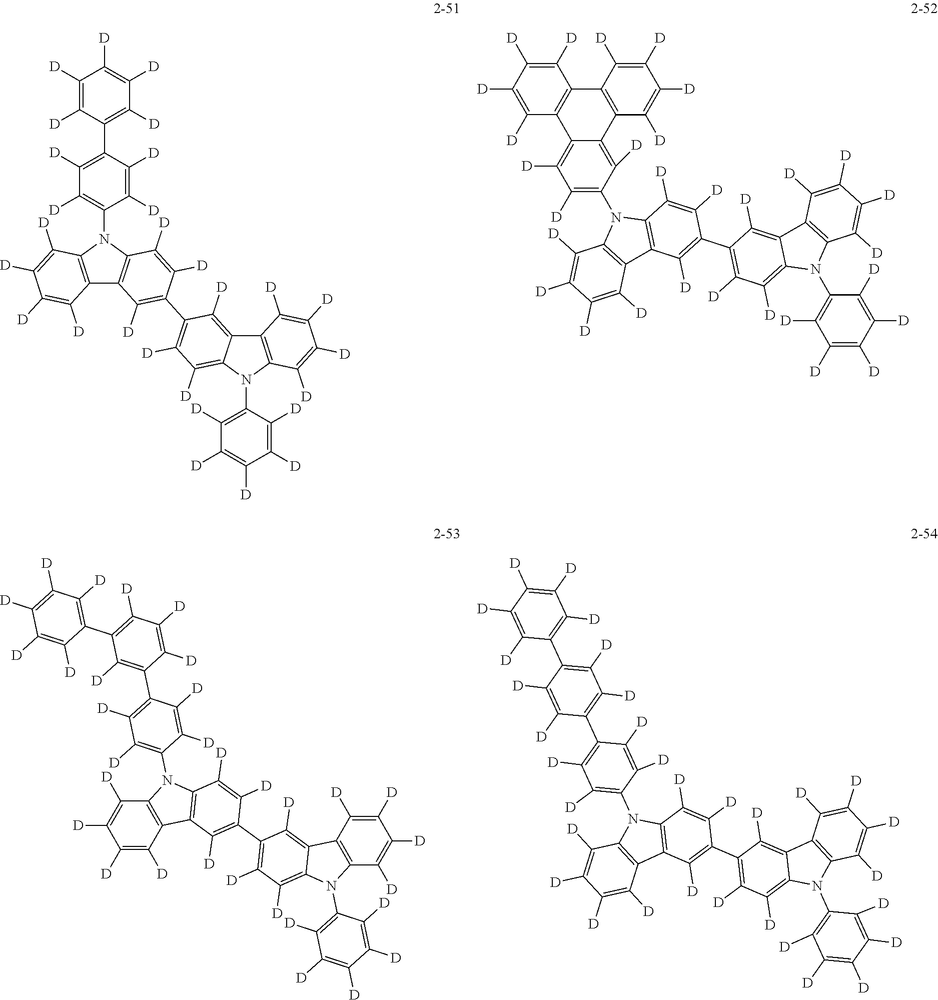

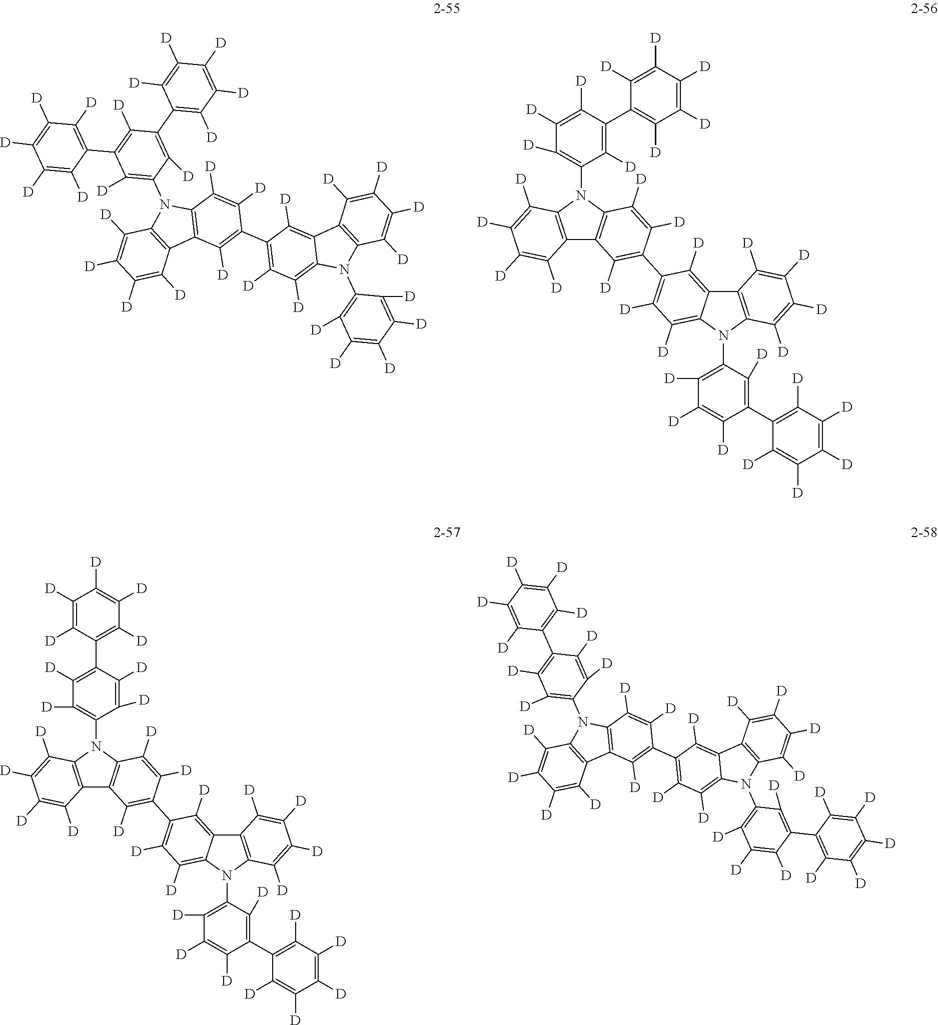

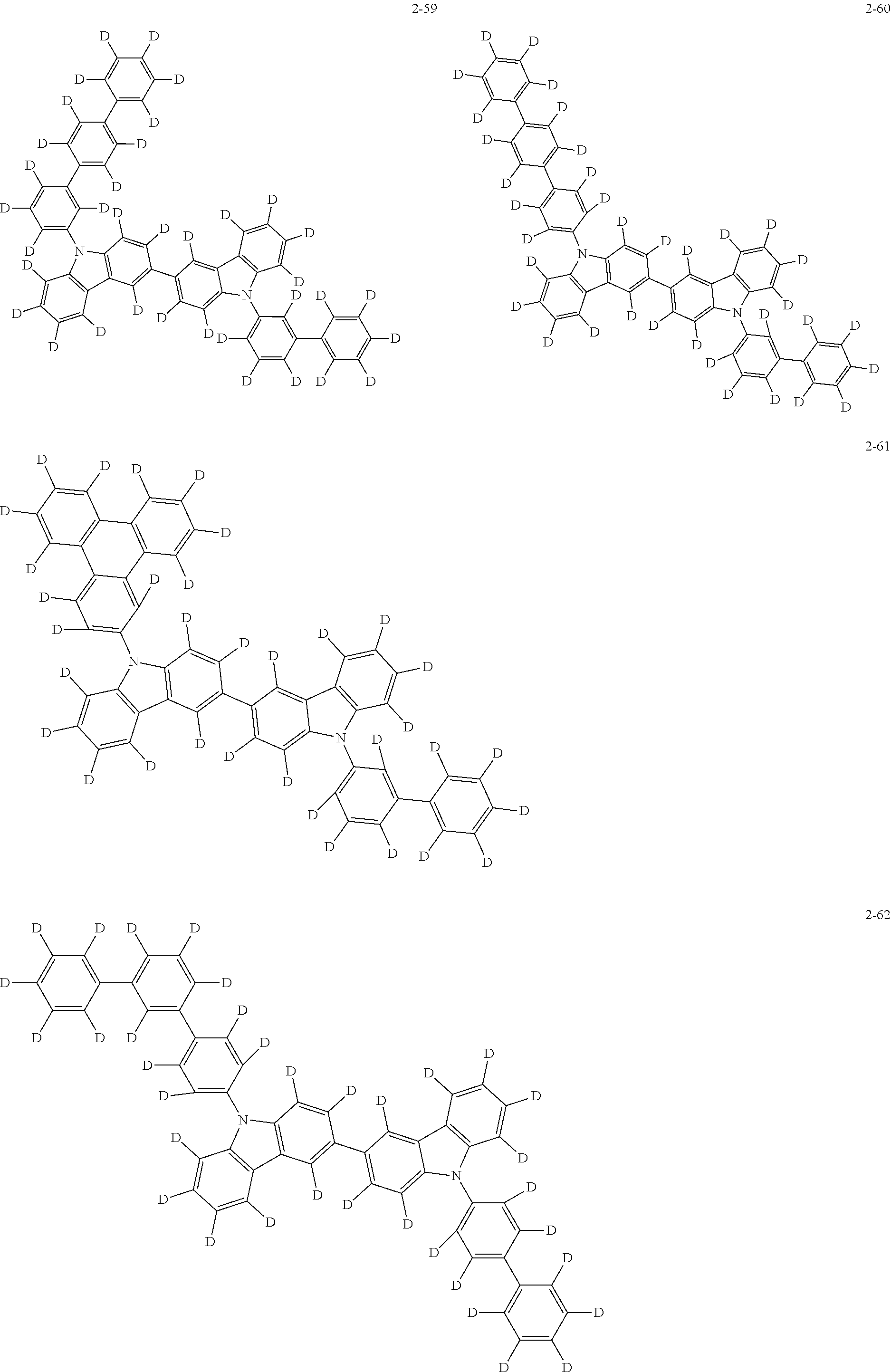

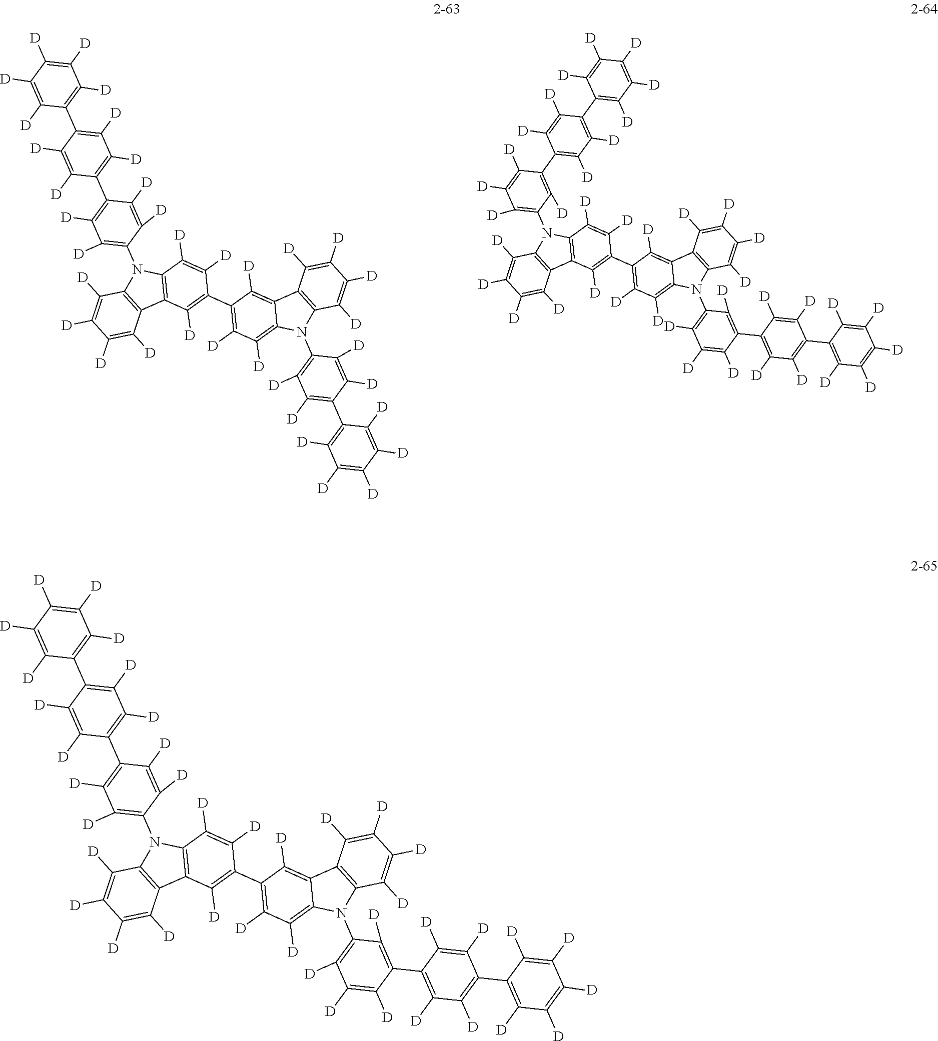

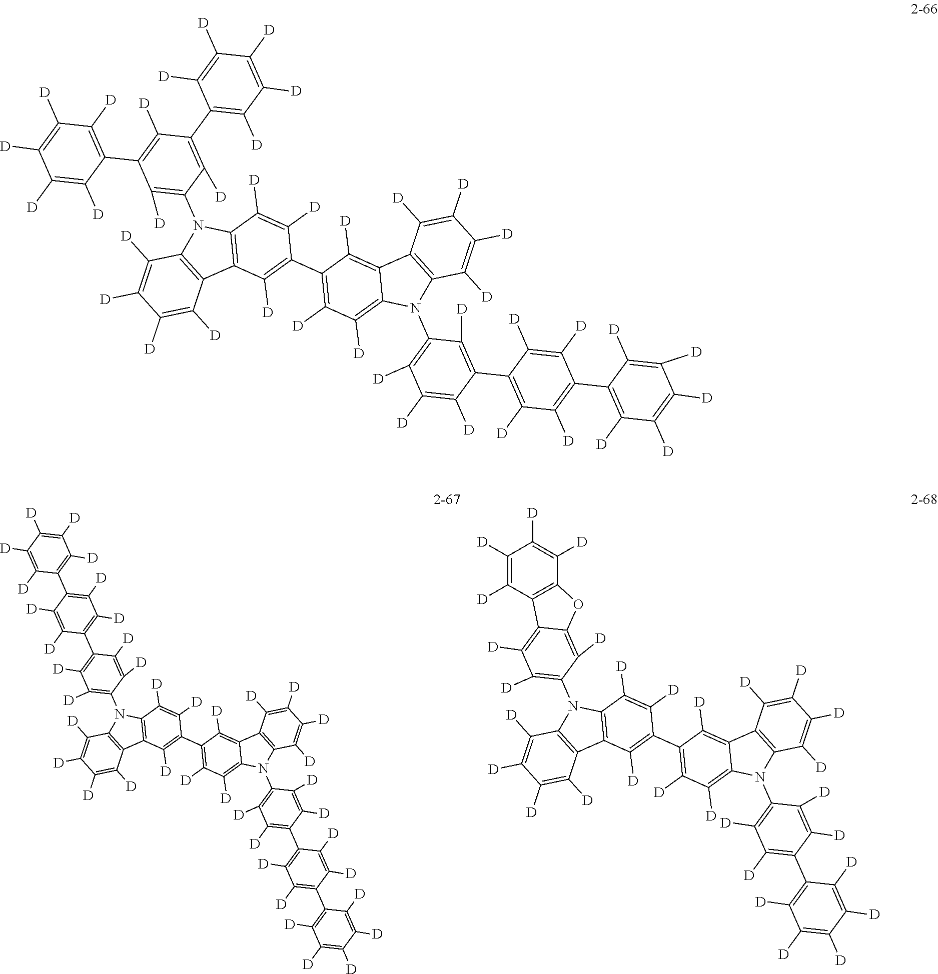

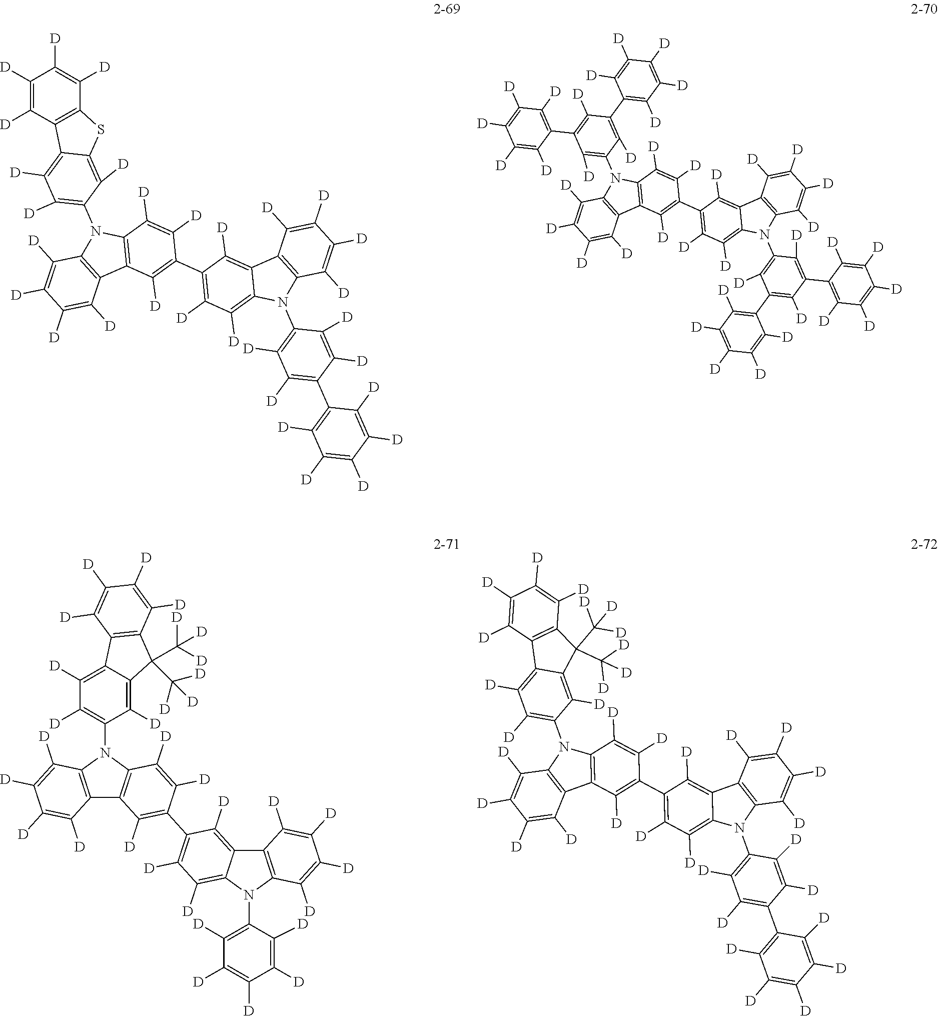

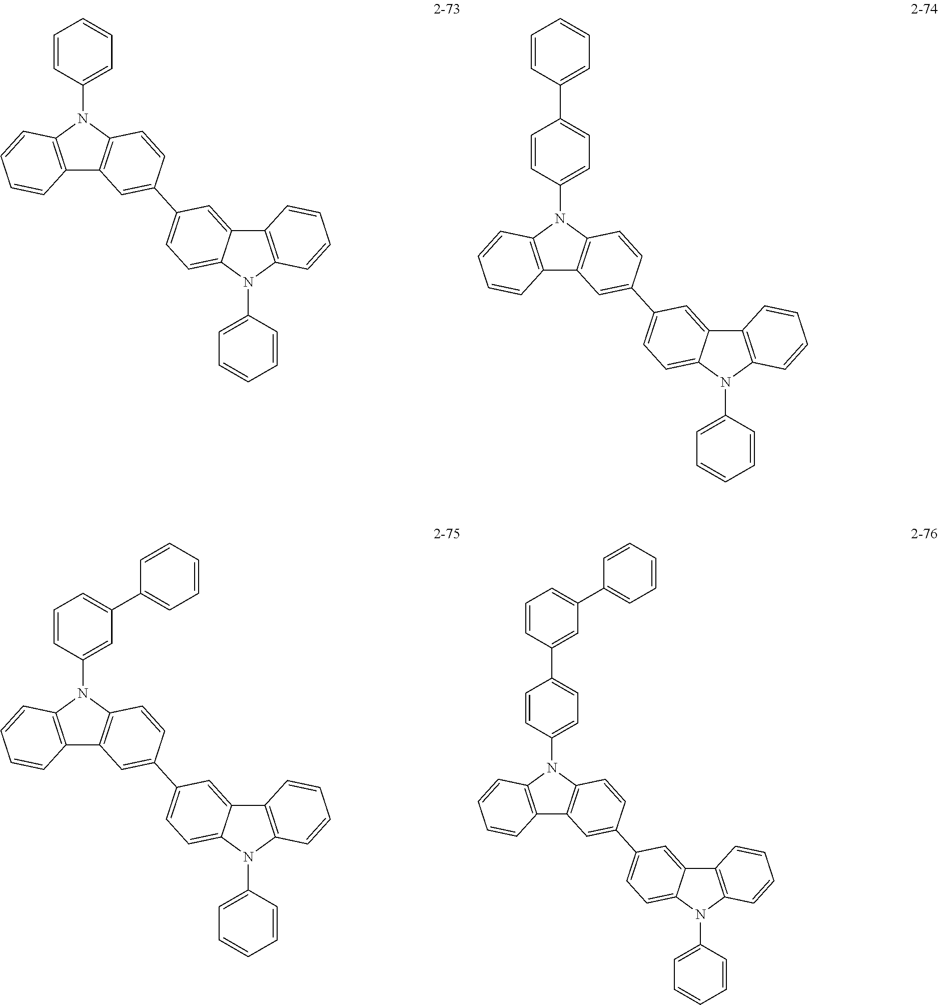 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

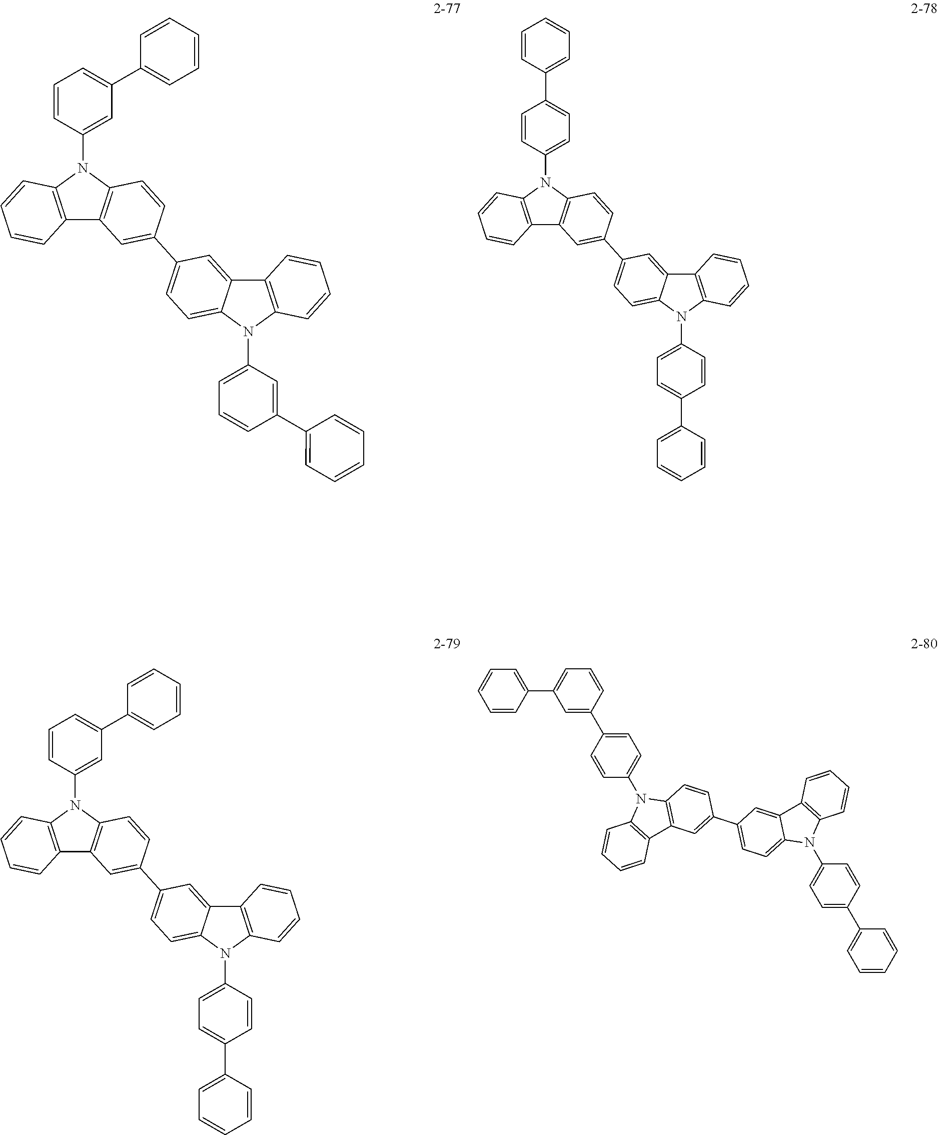 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1]

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present specification, the first electrode may be a positive electrode, and the second electrode may be a negative electrode.

In another exemplary embodiment of the present specification, the first electrode may be a negative electrode, and the second electrode may be a positive electrode.

The organic light emitting device according to an exemplary embodiment of the present specification may be manufactured by typical and materials for methods manufacturing an organic light emitting device, except that an organic material layer having one or more layers is formed by using the heterocyclic compound of the above-described Chemical Formula 1.

The heterocyclic compound of Chemical Formula 1 may be formed as an organic material layer by not only a vacuum deposition method, but also a solution application method when an organic light emitting device is manufactured. Herein, the solution coating method means spin coating, dip coating, inkjet printing, screen printing, a spray method, roll coating, and the like, but is not limited thereto.

In an exemplary embodiment of the present specification, the organic light emitting device may be a blue organic light emitting device, and the heterocyclic compound of Chemical Formula 1 may be used as a material for the blue organic light emitting device. For example, the heterocyclic compound of Chemical Formula 1 may be included in a light emitting layer of a blue organic light emitting device.

In another exemplary embodiment of the present specification, the organic light emitting device may be a green organic light emitting device, and the heterocyclic compound of Chemical Formula 1 may be used as a material for the green organic light emitting device. For example, the heterocyclic compound of Chemical Formula 1 may be included in a light emitting layer of a green organic light emitting device.

In still another exemplary embodiment of the present specification, the organic light emitting device may be a red organic light emitting device, and the heterocyclic compound of Chemical Formula 1 may be used as a material for the red organic light emitting device. For example, the heterocyclic compound of Chemical Formula 1 may be included in a light emitting layer of a red organic light emitting device.

The organic light emitting device of the present invention may further include one or more layers selected from the group consisting of a light emitting layer, a hole injection layer, a hole transport layer, an electron injection layer, an electron transport layer, an electron blocking layer, and a hole blocking layer.

FIGS. 1 to 3 exemplify the stacking sequence of the electrodes and the organic material layer of the organic light emitting device according to an exemplary embodiment of the present specification. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

An organic material layer including the heterocyclic compound of Chemical Formula 1 may additionally include other materials, if necessary.

In the organic light emitting device according to an exemplary embodiment of the present specification, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as Zno:Al or SnO₂:Sb; a conductive polymer such as poly(3-methylthiophene), poly[3,4-(ethylene-1,2-dioxy)thiophene] (PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrenesulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, any two or more materials from N-type host materials or P-type host materials may be selected and used as a host material for a light emitting layer.

The organic light emitting device according to an exemplary embodiment of the present specification may be a top emission type, a bottom emission type, or a dual emission type according to the material to be used.

The heterocyclic compound according to an exemplary embodiment of the present specification may act even in organic electronic devices including organic solar cells, organic photoconductors, organic transistors, and the like, based on the principle similar to those applied to organic light emitting devices.

In addition, it is possible to finely adjust an energy band-gap by introducing various substituents into the structure of Chemical Formula 1, and meanwhile, it is possible to improve characteristics at the interface between organic materials and diversify the use of the material.

In an exemplary embodiment of the present specification, provided is a composition for forming an organic material layer, including the heterocyclic compound.

In an exemplary embodiment of the present specification, the composition for forming an organic material layer may further include the compound of Chemical Formula 2.

In an exemplary embodiment of the present specification, the composition for forming an organic material layer may include the heterocyclic compound and the compound of Chemical Formula 2 as a weight ratio of 1:10 to 10:1.

In an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for forming an organic material layer, including the heterocyclic compound.

In an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for forming an organic material layer, including the heterocyclic compound and the compound of Chemical Formula 2 or 3.

In an exemplary embodiment of the present specification, provided is a method for manufacturing an organic light emitting device, in which the forming of the organic material layer forms the organic material layer by pre-mixing the heterocyclic compound represented by Chemical Formula 1 and the compound represented by Chemical Formula 2, and using a thermal vacuum deposition method.

The pre-mixing means that before the heterocyclic compound represented by Chemical Formula 1 and the compound represented by Chemical Formula 2 are deposited onto an organic material layer, the materials are first mixed and the mixture is contained in one common container and mixed.

The pre-mixed material may be referred to as a composition for forming an organic material layer according to an exemplary embodiment of the present specification.

Hereinafter, the present specification will be described in more detail through Examples, but these Examples are provided only for exemplifying the present application, and are not intended to limit the scope of the present application.

### MODE FOR INVENTION

### Preparation Examples

### <Preparation Example 1> Preparation of Compound 3

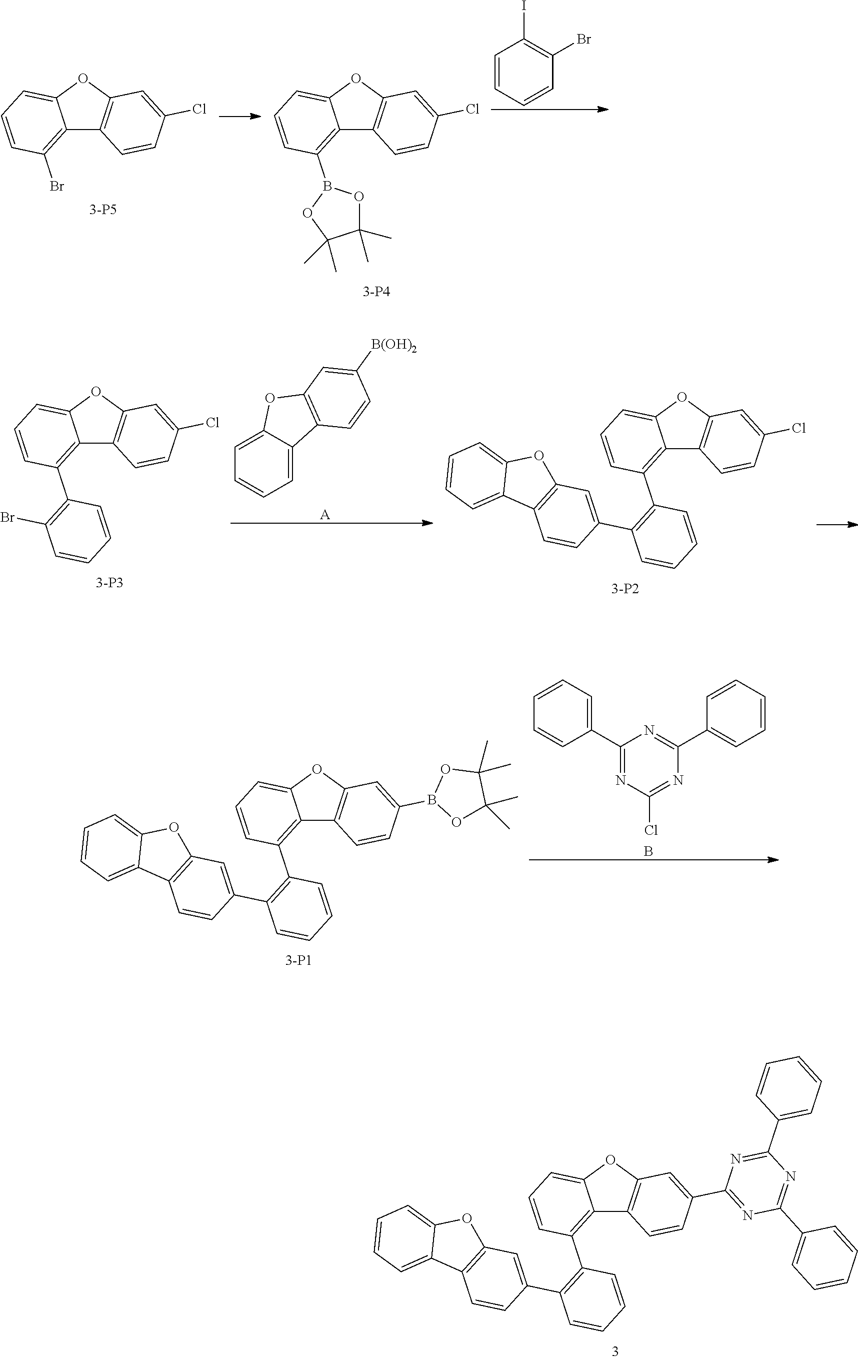

### 1) Preparation of Compound 3-P4

After 20 g (71.04 mmol) of Compound 3-P5 (1-bromo-7-chlorodibenzo[b,d]furan) and 27.06 g (106.56 mmol) of 4,4,4′,4′,5,5,5′,5′-octamethyl-2,2′-bi(1,3,2-dioxaborolane) were dissolved in 200 mL of 1,4-dioxane, 2.89 g (3.55 mmol) of [1,1′-bis(diphenylphosphino) ferrocene]dichloropalladium (II) (Pd(dppf)Cl₂) and 20.92 g (213.12 mmol) of potassium acetate were added thereto, and the resulting mixture was stirred under reflux for 16 hours. After the reaction was completed, ethyl acetate was added to the reaction solution for dissolution, and then the resulting solution was extracted with distilled water, the organic layer was dried over anhydrous MgSO₄, and then the solvent was removed using a rotary evaporator, and then the residue was purified by column chromatography using dichloromethane and hexane as eluting solvents, thereby obtaining 18.5 g (yield 79%) of Compound 3-P4.

### 2) Preparation of Compound 3-P3

After 18.5 g (56.30 mmol) of Compound 3-P4 and 15 g (56.30 mmol) of 1-bromo-2-iodobenzene were dissolved in 200 mL of 1,4-dioxane and 40 mL of distilled water, 3.25 g (2.81 mmol) of tetrakis(triphenylphosphine)palladium (0) (Pd(PPh₃)₄) and 19.45 g (140.75 mmol) of K₂CO₃ were added thereto, and the resulting mixture was stirred under reflux for 16 hours. After the reaction was completed, ethyl acetate was added to the reaction solution and dissolved, then the resulting solution was extracted with distilled water, the organic layer was dried over anhydrous MgSO₄, and then the solvent was removed using a rotary evaporator. Thereafter, purification was performed by column chromatography using dichloromethane and hexane as eluting solvents, thereby obtaining 16.1 g (yield 80%) of Compound 3-P3.

### 3) Preparation of Compound 3-P2

After 16.1 g (49.00 mmol) of Compound 3-P3 and 10.39 g (49.00 mmol) of dibenzo[b,d]furan-3-ylboronic acid (Compound A) were dissolved in 175 mL of 1,4-dioxane and 35 mL of distilled water, 3.25 g (2.45 mmol) of Pd(PPh₃)₄ and 16.93 g (122.49 mmol) of K₂CO₃ were added thereto, and the resulting mixture was stirred under reflux for 16 hours. After the reaction was completed, ethyl acetate was added to the reaction solution and dissolved, then the resulting solution was extracted with distilled water, the organic layer was dried over anhydrous MgSO₄, and then the solvent was removed using a rotary evaporator. Thereafter, purification was performed by column chromatography using dichloromethane and hexane as eluting solvents, thereby obtaining 14.5 g (yield 66%) of Compound 3-P2.

### 4) Preparation of Compound 3-P1

After 14.5 g (32.59 mmol) of Compound 3-P2 and 12.41 g (48.89 mmol) of 4,4,4′,4′,5,5,5′,5′-octamethyl-2,2′-bi(1,3,2-dioxaborolane) were dissolved in 150 mL of 1,4-dioxane, 1.49 g (1.63 mmol) of tris(dibenzylideneacetone) dipalladium (0) (Pd₂ (dba)₃), 9.6 g (97.77 mmol) of potassium acetate, and 1.55 g (3.26 mmol) of 2-dicyclohexylphosphino-2,4′,6-triisopropylbiphenyl (Xphos) were added thereto, and the resulting mixture was stirred under reflux for 16 hours. After the reaction was completed, ethyl acetate was added to the reaction solution for dissolution, and then the resulting solution was extracted with distilled water, the organic layer was dried over anhydrous MgSO₄, and then the solvent was removed using a rotary evaporator, and then the residue was purified by column chromatography using dichloromethane and hexane as eluting solvents, thereby obtaining 13.1 g (yield 75%) of Compound 3-P1.

### 5) Preparation of Compound 3

After 13.1 g (24.42 mmol) of Compound 3-P1 and 6.54 g (24.42 mmol) of 2-chloro-4,6-diphenyl-1,3,5-triazine (Compound B) were dissolved in 150 mL of 1,4-dioxane and 30 mL of distilled water, 1.41 g (1.22 mmol) of Pd(PPh₃)₄ and 8.44 g (61.05 mmol) of K₂CO₃ were added thereto, and the resulting mixture was stirred under reflux for 16 hours. After the reaction was completed, ethyl acetate was added to the reaction solution and dissolved, then the resulting solution was extracted with distilled water, the organic layer was dried over anhydrous MgSO₄, and then the solvent was removed using a rotary evaporator. Thereafter, purification was performed by column chromatography using dichloromethane and hexane as eluting solvents, thereby obtaining 11.45 g (yield 73%) of Compound 3.

Target compounds in the following Table 1 were synthesized by performing the preparation in the same manner as in Preparation Example 1, except that Compound P5′ in the following Table 1 was used instead of Compound 3-P5, Compound A′ in the following Table 1 was used instead of Compound A, and Compound B′ in the following Table 1 was used instead of Compound B.

TABLE 1

| Com- | | | | | |
|---|---|---|---|---|---|
| pound | Compound | Compound | | Target | |
| No. | P5′ | A′ | Compound B′ | Compound | Yield |
| 1 | 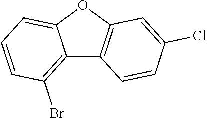 [SMILES: Clc1ccc2c(c1)oc1cccc(Br)c12] | 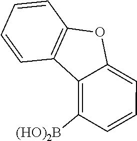 | 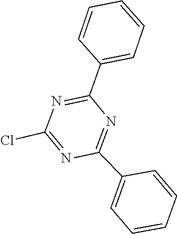 [SMILES: Clc1nc(-c2ccccc2)nc(-c2ccccc2)n1] | 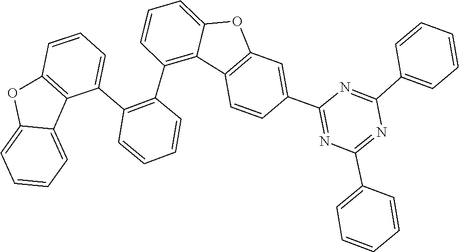 [SMILES: c1ccc(-c2nc(-c3ccccc3)nc(-c3ccc4c(c3)oc3cccc(-c5ccccc5-c5cccc6oc7ccccc7c56)c34)n2)cc1] | 70% |
| 5 | 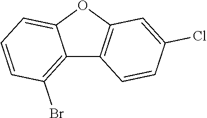 [SMILES: Clc1ccc2c(c1)oc1cccc(Br)c12] | 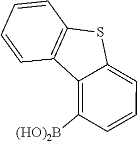 | 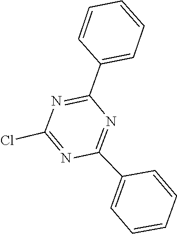 [SMILES: Clc1nc(-c2ccccc2)nc(-c2ccccc2)n1] | 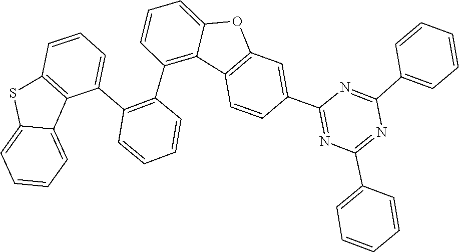 [SMILES: c1ccc(-c2nc(-c3ccccc3)nc(-c3ccc4c(c3)oc3cccc(-c5ccccc5-c5cccc6sc7ccccc7c56)c34)n2)cc1] | 71% |
| 8 | 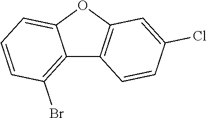 [SMILES: Clc1ccc2c(c1)oc1cccc(Br)c12] | 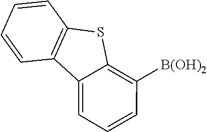 [SMILES: OB(O)c1cccc2c1sc1ccccc12] | 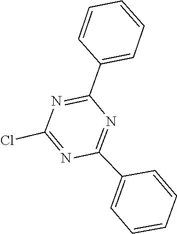 [SMILES: Clc1nc(-c2ccccc2)nc(-c2ccccc2)n1] | 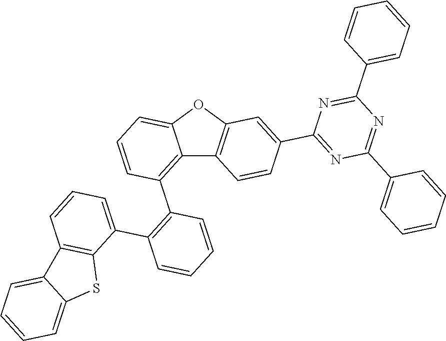 [SMILES: c1ccc(-c2nc(-c3ccccc3)nc(-c3ccc4c(c3)oc3cccc(-c5ccccc5-c5cccc6c5sc5ccccc56)c34)n2)cc1] | 69% |
| 10 | 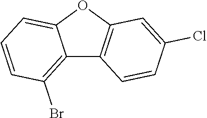 [SMILES: Clc1ccc2c(c1)oc1cccc(Br)c12] | 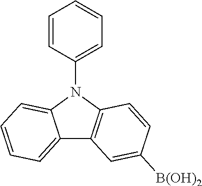 [SMILES: OB(O)c1ccc2c(c1)c1ccccc1n2-c1ccccc1] | 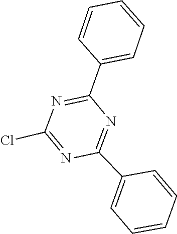 [SMILES: Clc1nc(-c2ccccc2)nc(-c2ccccc2)n1] | 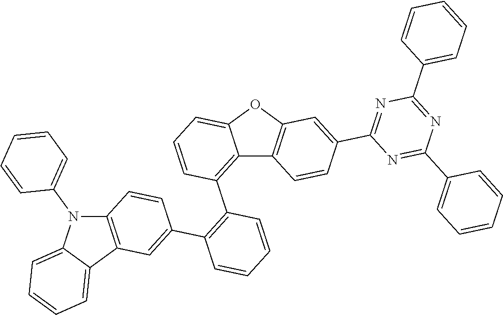 [SMILES: c1ccc(-c2nc(-c3ccccc3)nc(-c3ccc4c(c3)oc3cccc(-c5ccccc5-c5ccc6c(c5)c5ccccc5n6-c5ccccc5)c34)n2)cc1] | 67% |
| 13 | 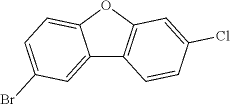 [SMILES: Clc1ccc2c(c1)oc1ccc(Br)cc12] | 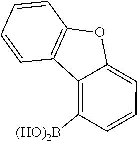 | 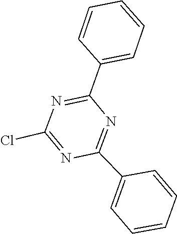 [SMILES: Clc1nc(-c2ccccc2)nc(-c2ccccc2)n1] | 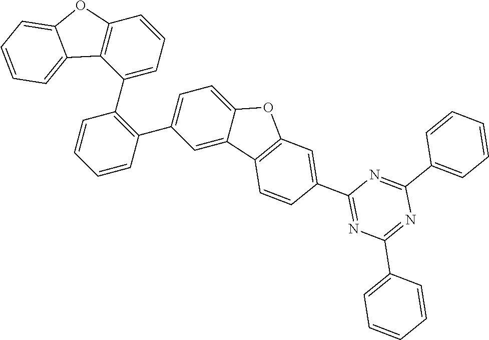 [SMILES: c1ccc(-c2nc(-c3ccccc3)nc(-c3ccc4c(c3)oc3ccc(-c5ccccc5-c5cccc6oc7ccccc7c56)cc34)n2)cc1] | 71% |
| 16 | 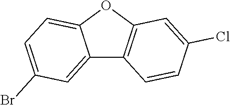 [SMILES: Clc1ccc2c(c1)oc1ccc(Br)cc12] | 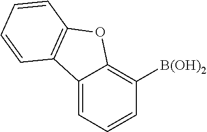 [SMILES: OB(O)c1cccc2c1oc1ccccc12] | 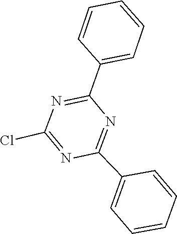 [SMILES: Clc1nc(-c2ccccc2)nc(-c2ccccc2)n1] | 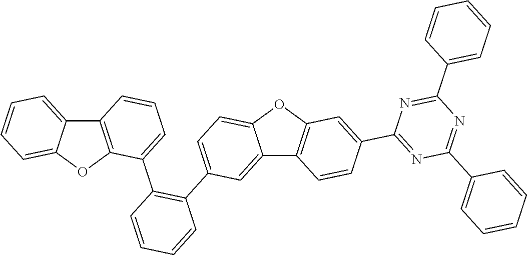 [SMILES: c1ccc(-c2nc(-c3ccccc3)nc(-c3ccc4c(c3)oc3ccc(-c5ccccc5-c5cccc6c5oc5ccccc56)cc34)n2)cc1] | 70% |
| 20 | 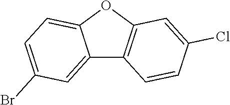 [SMILES: Clc1ccc2c(c1)oc1ccc(Br)cc12] | 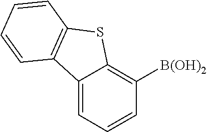 [SMILES: OB(O)c1cccc2c1sc1ccccc12] | 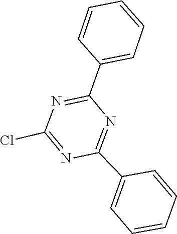 [SMILES: Clc1nc(-c2ccccc2)nc(-c2ccccc2)n1] | 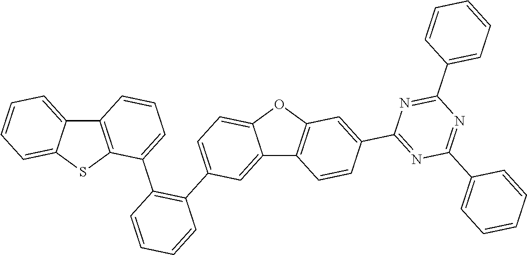 [SMILES: c1ccc(-c2nc(-c3ccccc3)nc(-c3ccc4c(c3)oc3ccc(-c5ccccc5-c5cccc6c5sc5ccccc56)cc34)n2)cc1] | 71% |
| 24 | 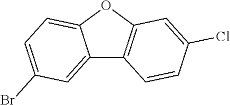 [SMILES: Clc1ccc2c(c1)oc1ccc(Br)cc12] | 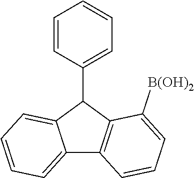 [SMILES: OB(O)c1cccc2c1C(c1ccccc1)c1ccccc1-2] | 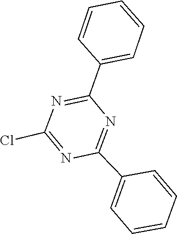 [SMILES: Clc1nc(-c2ccccc2)nc(-c2ccccc2)n1] | 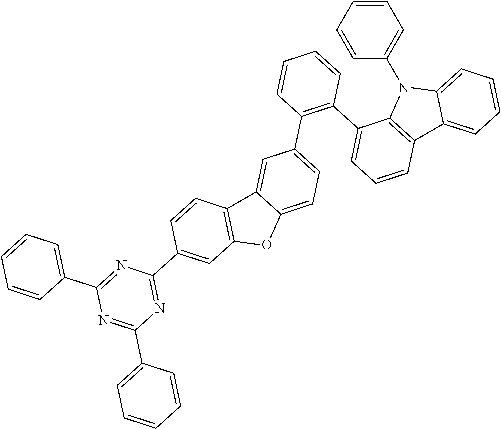 [SMILES: c1ccc(-c2nc(-c3ccccc3)nc(-c3ccc4c(c3)oc3ccc(-c5ccccc5-c5cccc6c7ccccc7n(-c7ccccc7)c56)cc34)n2)cc1] | 68% |
| 25 | 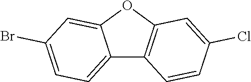 [SMILES: Clc1ccc2c(c1)oc1cc(Br)ccc12] | 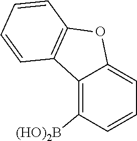 | 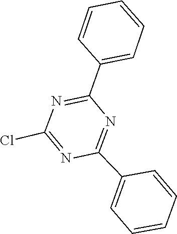 [SMILES: Clc1nc(-c2ccccc2)nc(-c2ccccc2)n1] | 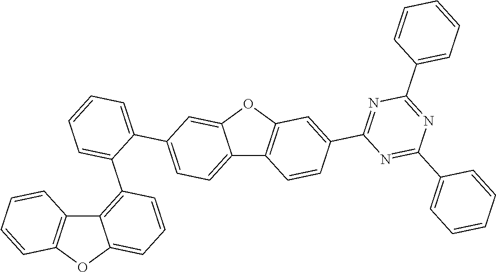 [SMILES: c1ccc(-c2nc(-c3ccccc3)nc(-c3ccc4c(c3)oc3cc(-c5ccccc5-c5cccc6oc7ccccc7c56)ccc34)n2)cc1] | 69% |
| 32 | 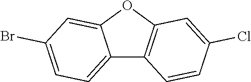 [SMILES: Clc1ccc2c(c1)oc1cc(Br)ccc12] | 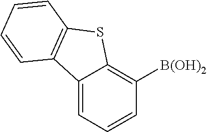 [SMILES: OB(O)c1cccc2c1sc1ccccc12] | 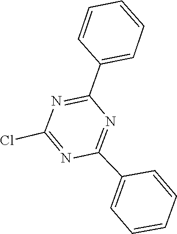 [SMILES: Clc1nc(-c2ccccc2)nc(-c2ccccc2)n1] | 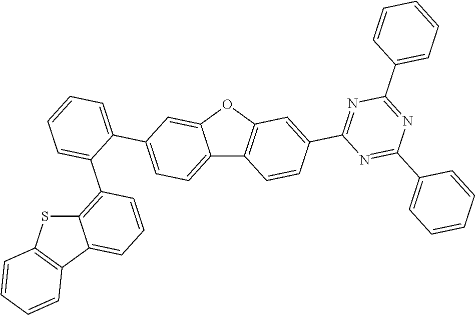 [SMILES: c1ccc(-c2nc(-c3ccccc3)nc(-c3ccc4c(c3)oc3cc(-c5ccccc5-c5cccc6c5sc5ccccc56)ccc34)n2)cc1] | 70% |
| 34 | 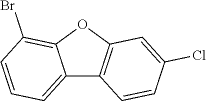 [SMILES: Clc1ccc2c(c1)oc1c(Br)cccc12] | 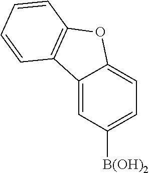 [SMILES: OB(O)c1ccc2oc3ccccc3c2c1] | 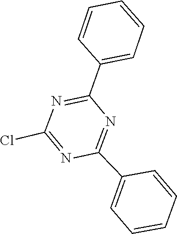 [SMILES: Clc1nc(-c2ccccc2)nc(-c2ccccc2)n1] | 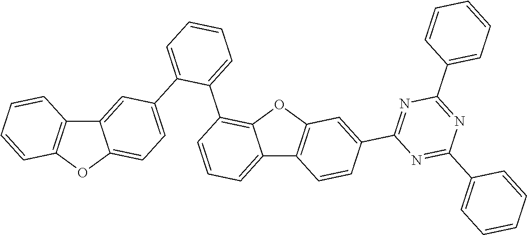 [SMILES: c1ccc(-c2nc(-c3ccccc3)nc(-c3ccc4c(c3)oc3c(-c5ccccc5-c5ccc6oc7ccccc7c6c5)cccc34)n2)cc1] | 72% |
| 40 | 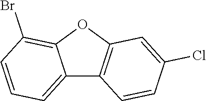 [SMILES: Clc1ccc2c(c1)oc1c(Br)cccc12] | 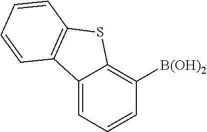 [SMILES: OB(O)c1cccc2c1sc1ccccc12] | 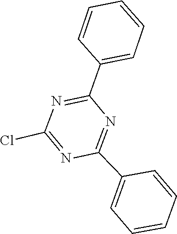 [SMILES: Clc1nc(-c2ccccc2)nc(-c2ccccc2)n1] | 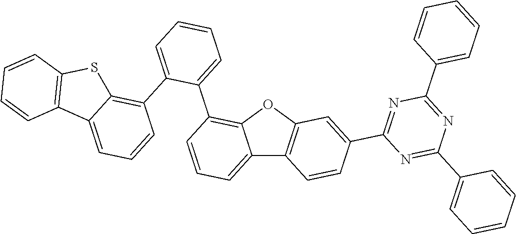 [SMILES: c1ccc(-c2nc(-c3ccccc3)nc(-c3ccc4c(c3)oc3c(-c5ccccc5-c5cccc6c5sc5ccccc56)cccc34)n2)cc1] | 71% |
| 53 | 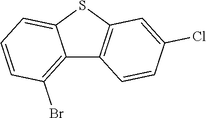 [SMILES: Clc1ccc2c(c1)sc1cccc(Br)c12] | 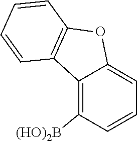 | 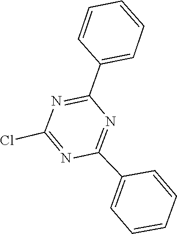 [SMILES: Clc1nc(-c2ccccc2)nc(-c2ccccc2)n1] | 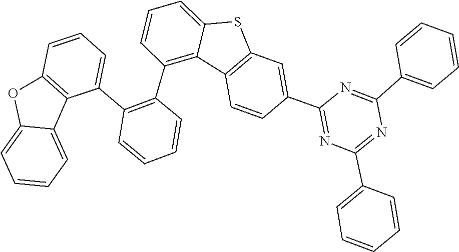 [SMILES: c1ccc(-c2nc(-c3ccccc3)nc(-c3ccc4c(c3)sc3cccc(-c5ccccc5-c5cccc6oc7ccccc7c56)c34)n2)cc1] | 67% |
| 56 | 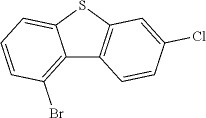 [SMILES: Clc1ccc2c(c1)sc1cccc(Br)c12] | 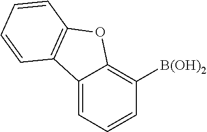 [SMILES: OB(O)c1cccc2c1oc1ccccc12] | 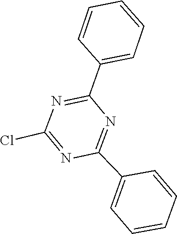 [SMILES: Clc1nc(-c2ccccc2)nc(-c2ccccc2)n1] | 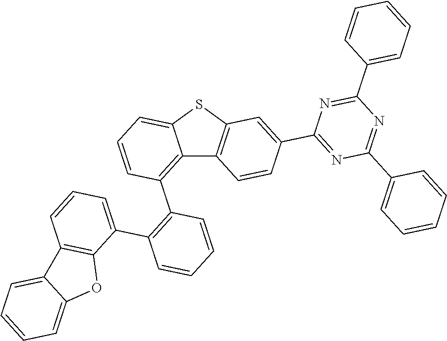 [SMILES: c1ccc(-c2nc(-c3ccccc3)nc(-c3ccc4c(c3)sc3cccc(-c5ccccc5-c5cccc6c5oc5ccccc56)c34)n2)cc1] | 69% |
| 57 | 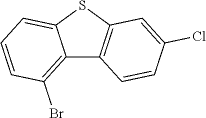 [SMILES: Clc1ccc2c(c1)sc1cccc(Br)c12] | 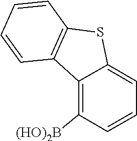 | 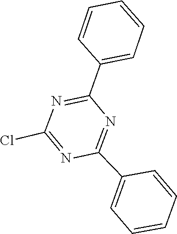 [SMILES: Clc1nc(-c2ccccc2)nc(-c2ccccc2)n1] | 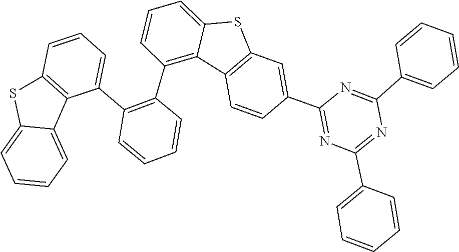 [SMILES: c1ccc(-c2nc(-c3ccccc3)nc(-c3ccc4c(c3)sc3cccc(-c5ccccc5-c5cccc6sc7ccccc7c56)c34)n2)cc1] | 70% |
| 60 | 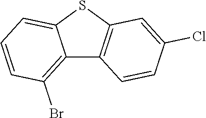 [SMILES: Clc1ccc2c(c1)sc1cccc(Br)c12] | 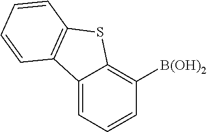 [SMILES: OB(O)c1cccc2c1sc1ccccc12] | 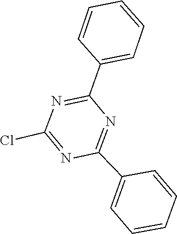 [SMILES: Clc1nc(-c2ccccc2)nc(-c2ccccc2)n1] | 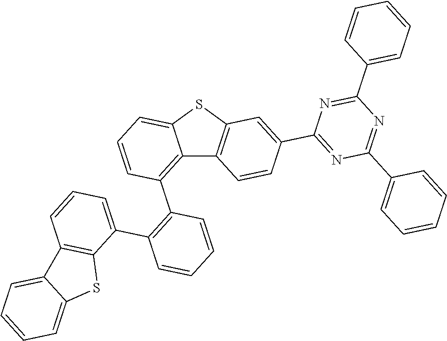 [SMILES: c1ccc(-c2nc(-c3ccccc3)nc(-c3ccc4c(c3)sc3cccc(-c5ccccc5-c5cccc6c5sc5ccccc56)c34)n2)cc1] | 71% |
| 64 | 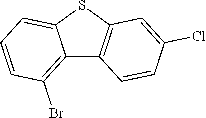 [SMILES: Clc1ccc2c(c1)sc1cccc(Br)c12] | 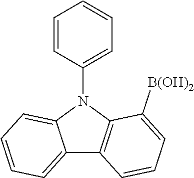 [SMILES: OB(O)c1cccc2c3ccccc3n(-c3ccccc3)c12] | 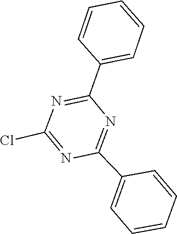 [SMILES: Clc1nc(-c2ccccc2)nc(-c2ccccc2)n1] | 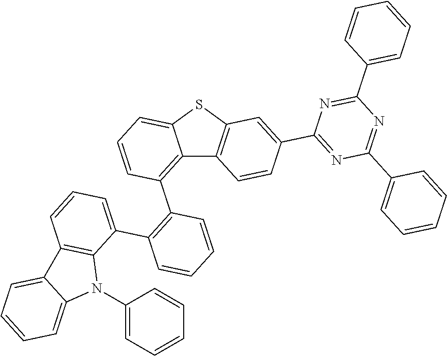 [SMILES: c1ccc(-c2nc(-c3ccccc3)nc(-c3ccc4c(c3)sc3cccc(-c5ccccc5-c5cccc6c7ccccc7n(-c7ccccc7)c56)c34)n2)cc1] | 72% |
| 101 | 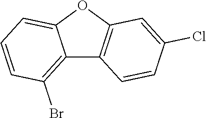 [SMILES: Clc1ccc2c(c1)oc1cccc(Br)c12] | 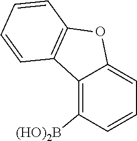 | 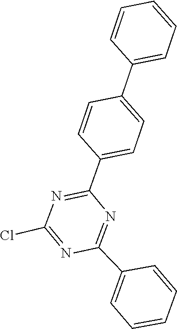 [SMILES: Clc1nc(-c2ccccc2)nc(-c2ccc(-c3ccccc3)cc2)n1] | 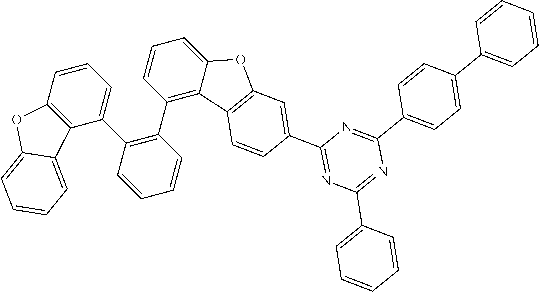 [SMILES: c1ccc(-c2ccc(-c3nc(-c4ccccc4)nc(-c4ccc5c(c4)oc4cccc(-c6ccccc6-c6cccc7oc8ccccc8c67)c45)n3)cc2)cc1] | 70% |
| 115 | 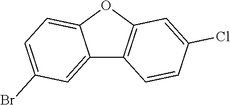 [SMILES: Clc1ccc2c(c1)oc1ccc(Br)cc12] | 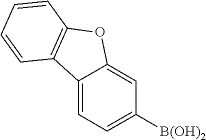 [SMILES: OB(O)c1ccc2c(c1)oc1ccccc12] | 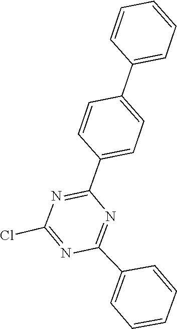 [SMILES: Clc1nc(-c2ccccc2)nc(-c2ccc(-c3ccccc3)cc2)n1] | 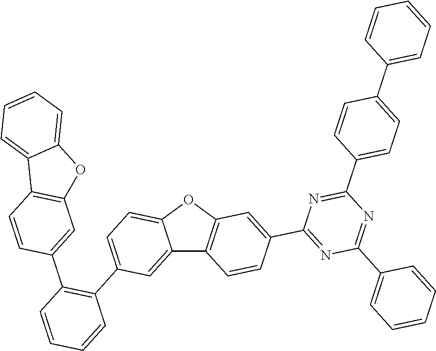 [SMILES: c1ccc(-c2ccc(-c3nc(-c4ccccc4)nc(-c4ccc5c(c4)oc4ccc(-c6ccccc6-c6ccc7c(c6)oc6ccccc67)cc45)n3)cc2)cc1] | 68% |
| 132 | 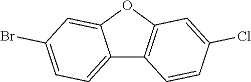 [SMILES: Clc1ccc2c(c1)oc1cc(Br)ccc12] | 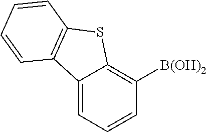 [SMILES: OB(O)c1cccc2c1sc1ccccc12] | 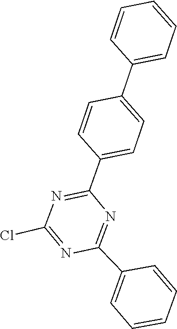 [SMILES: Clc1nc(-c2ccccc2)nc(-c2ccc(-c3ccccc3)cc2)n1] | 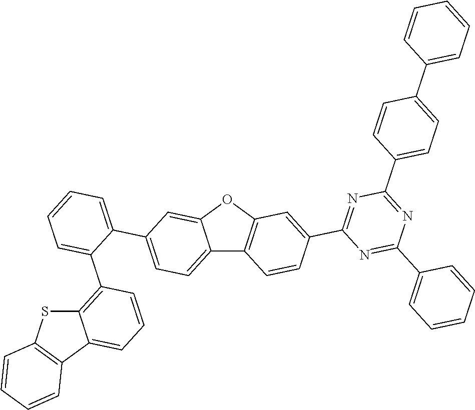 [SMILES: c1ccc(-c2ccc(-c3nc(-c4ccccc4)nc(-c4ccc5c(c4)oc4cc(-c6ccccc6-c6cccc7c6sc6ccccc67)ccc45)n3)cc2)cc1] | 69% |
| 136 | 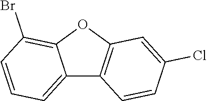 [SMILES: Clc1ccc2c(c1)oc1c(Br)cccc12] | 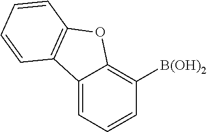 [SMILES: OB(O)c1cccc2c1oc1ccccc12] | 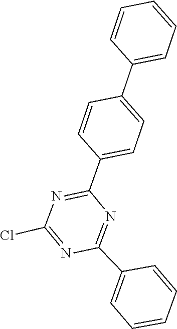 [SMILES: Clc1nc(-c2ccccc2)nc(-c2ccc(-c3ccccc3)cc2)n1] | 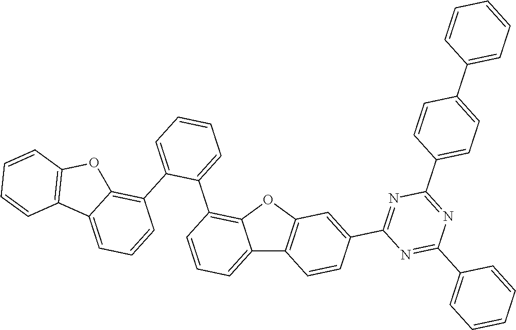 [SMILES: c1ccc(-c2ccc(-c3nc(-c4ccccc4)nc(-c4ccc5c(c4)oc4c(-c6ccccc6-c6cccc7c6oc6ccccc67)cccc45)n3)cc2)cc1] | 70% |
| 153 | 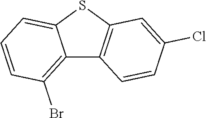 [SMILES: Clc1ccc2c(c1)sc1cccc(Br)c12] | 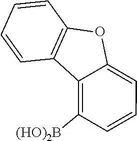 | 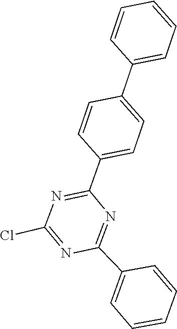 [SMILES: Clc1nc(-c2ccccc2)nc(-c2ccc(-c3ccccc3)cc2)n1] | 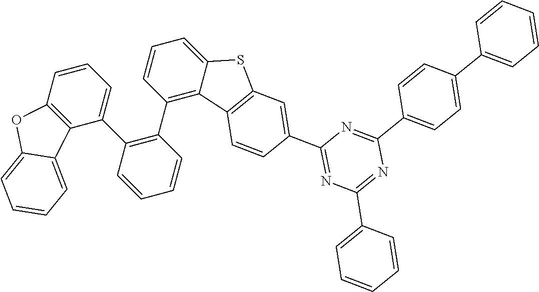 [SMILES: c1ccc(-c2ccc(-c3nc(-c4ccccc4)nc(-c4ccc5c(c4)sc4cccc(-c6ccccc6-c6cccc7oc8ccccc8c67)c45)n3)cc2)cc1] | 71% |
| 160 | 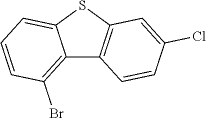 [SMILES: Clc1ccc2c(c1)sc1cccc(Br)c12] | 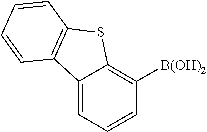 [SMILES: OB(O)c1cccc2c1sc1ccccc12] | 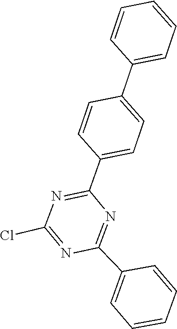 [SMILES: Clc1nc(-c2ccccc2)nc(-c2ccc(-c3ccccc3)cc2)n1] | 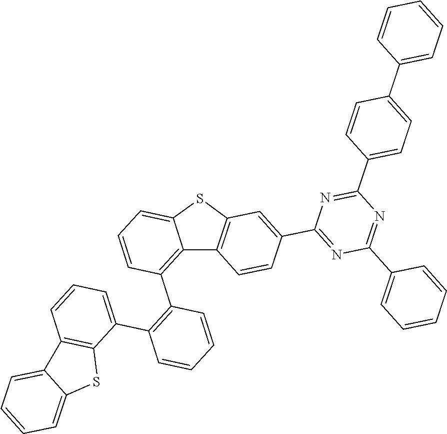 [SMILES: c1ccc(-c2ccc(-c3nc(-c4ccccc4)nc(-c4ccc5c(c4)sc4cccc(-c6ccccc6-c6cccc7c6sc6ccccc67)c45)n3)cc2)cc1] | 72% |
| 165 | 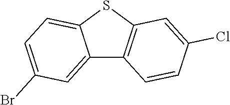 [SMILES: Clc1ccc2c(c1)sc1ccc(Br)cc12] | 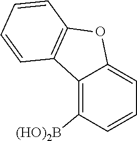 | 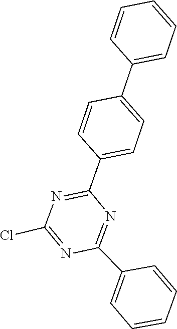 [SMILES: Clc1nc(-c2ccccc2)nc(-c2ccc(-c3ccccc3)cc2)n1] | 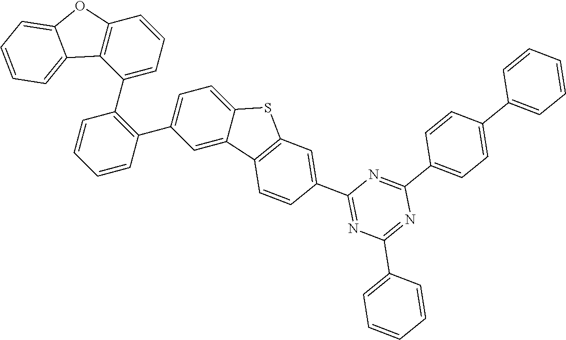 [SMILES: c1ccc(-c2ccc(-c3nc(-c4ccccc4)nc(-c4ccc5c(c4)sc4ccc(-c6ccccc6-c6cccc7oc8ccccc8c67)cc45)n3)cc2)cc1] | 71% |
| 180 | 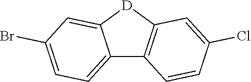 | 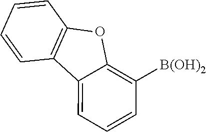 [SMILES: OB(O)c1cccc2c1oc1ccccc12] | 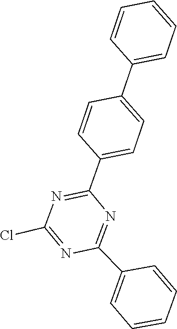 [SMILES: Clc1nc(-c2ccccc2)nc(-c2ccc(-c3ccccc3)cc2)n1] | 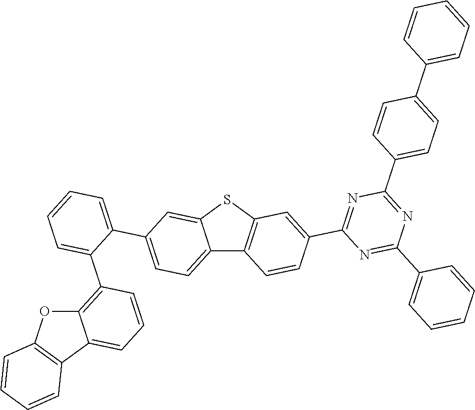 [SMILES: c1ccc(-c2ccc(-c3nc(-c4ccccc4)nc(-c4ccc5c(c4)sc4cc(-c6ccccc6-c6cccc7c6oc6ccccc67)ccc45)n3)cc2)cc1] | 70% |
| 203 | 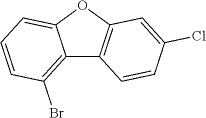 [SMILES: Clc1ccc2c(c1)oc1cccc(Br)c12] | 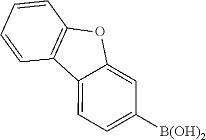 [SMILES: OB(O)c1ccc2c(c1)oc1ccccc12] | 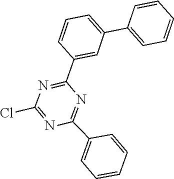 [SMILES: Clc1nc(-c2ccccc2)nc(-c2cccc(-c3ccccc3)c2)n1] | 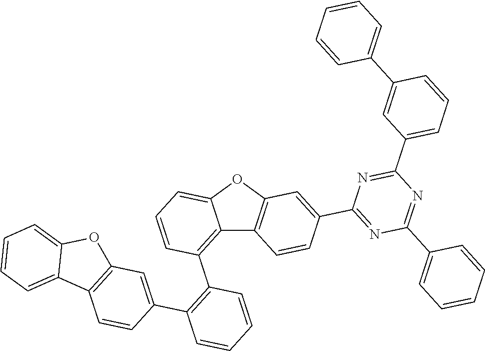 [SMILES: c1ccc(-c2cccc(-c3nc(-c4ccccc4)nc(-c4ccc5c(c4)oc4cccc(-c6ccccc6-c6ccc7c(c6)oc6ccccc67)c45)n3)c2)cc1] | 69% |
| 220 | 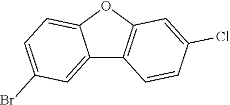 [SMILES: Clc1ccc2c(c1)oc1ccc(Br)cc12] | 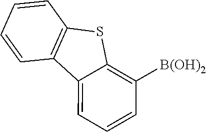 [SMILES: OB(O)c1cccc2c1sc1ccccc12] | 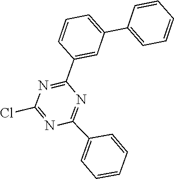 [SMILES: Clc1nc(-c2ccccc2)nc(-c2cccc(-c3ccccc3)c2)n1] | 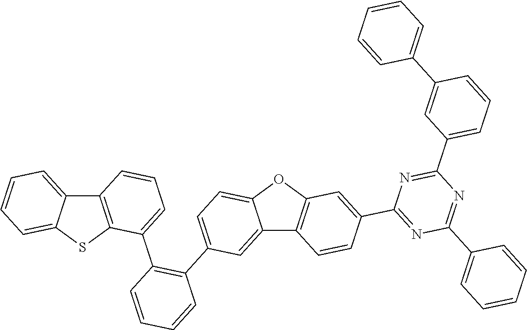 [SMILES: c1ccc(-c2cccc(-c3nc(-c4ccccc4)nc(-c4ccc5c(c4)oc4ccc(-c6ccccc6-c6cccc7c6sc6ccccc67)cc45)n3)c2)cc1] | 68% |
| 226 | 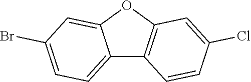 [SMILES: Clc1ccc2c(c1)oc1cc(Br)ccc12] | 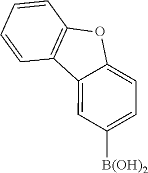 [SMILES: OB(O)c1ccc2oc3ccccc3c2c1] | 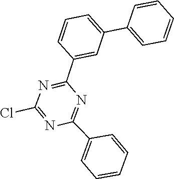 [SMILES: Clc1nc(-c2ccccc2)nc(-c2cccc(-c3ccccc3)c2)n1] | 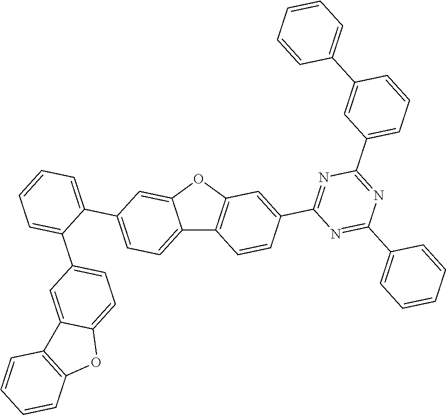 [SMILES: c1ccc(-c2cccc(-c3nc(-c4ccccc4)nc(-c4ccc5c(c4)oc4cc(-c6ccccc6-c6ccc7oc8ccccc8c7c6)ccc45)n3)c2)cc1] | 67% |
| 254 | 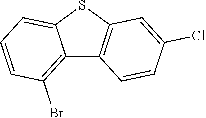 [SMILES: Clc1ccc2c(c1)sc1cccc(Br)c12] | 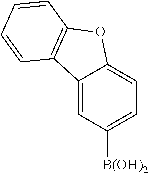 [SMILES: OB(O)c1ccc2oc3ccccc3c2c1] | 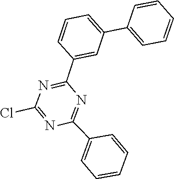 [SMILES: Clc1nc(-c2ccccc2)nc(-c2cccc(-c3ccccc3)c2)n1] | 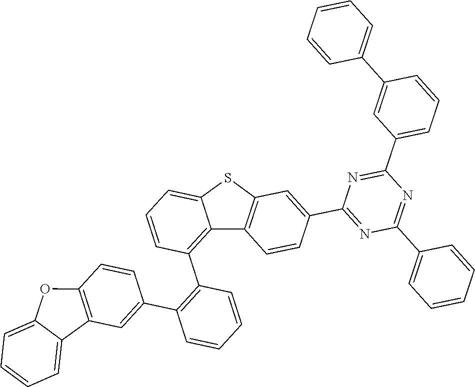 [SMILES: c1ccc(-c2cccc(-c3nc(-c4ccccc4)nc(-c4ccc5c(c4)sc4cccc(-c6ccccc6-c6ccc7oc8ccccc8c7c6)c45)n3)c2)cc1] | 72% |
| 301 | 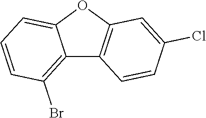 [SMILES: Clc1ccc2c(c1)oc1cccc(Br)c12] | 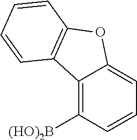 | 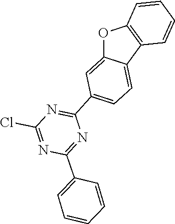 [SMILES: Clc1nc(-c2ccccc2)nc(-c2ccc3c(c2)oc2ccccc23)n1] | 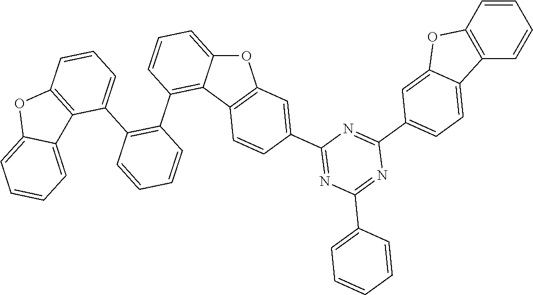 [SMILES: c1ccc(-c2nc(-c3ccc4c(c3)oc3ccccc34)nc(-c3ccc4c(c3)oc3cccc(-c5ccccc5-c5cccc6oc7ccccc7c56)c34)n2)cc1] | 70% |
| 313 | 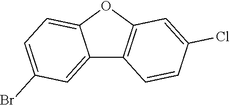 [SMILES: Clc1ccc2c(c1)oc1ccc(Br)cc12] | 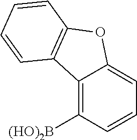 | 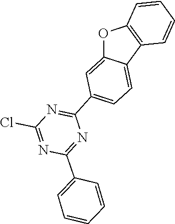 [SMILES: Clc1nc(-c2ccccc2)nc(-c2ccc3c(c2)oc2ccccc23)n1] | 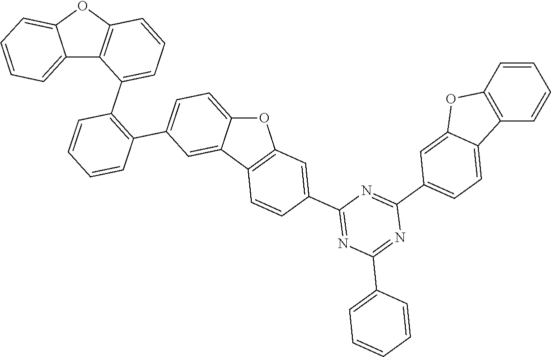 [SMILES: c1ccc(-c2nc(-c3ccc4c(c3)oc3ccccc34)nc(-c3ccc4c(c3)oc3ccc(-c5ccccc5-c5cccc6oc7ccccc7c56)cc34)n2)cc1] | 68% |
| 327 | 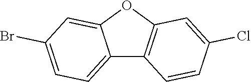 [SMILES: Clc1ccc2c(c1)oc1cc(Br)ccc12] | 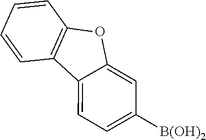 [SMILES: OB(O)c1ccc2c(c1)oc1ccccc12] | 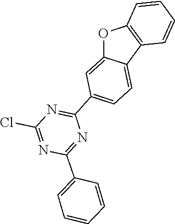 [SMILES: Clc1nc(-c2ccccc2)nc(-c2ccc3c(c2)oc2ccccc23)n1] | 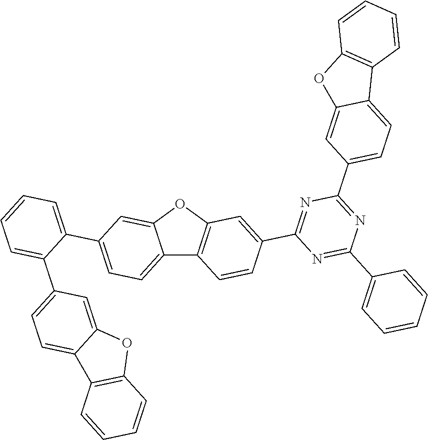 [SMILES: c1ccc(-c2nc(-c3ccc4c(c3)oc3ccccc34)nc(-c3ccc4c(c3)oc3cc(-c5ccccc5-c5ccc6c(c5)oc5ccccc56)ccc34)n2)cc1] | 67% |
| 338 | 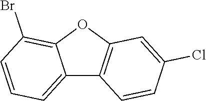 [SMILES: Clc1ccc2c(c1)oc1c(Br)cccc12] | 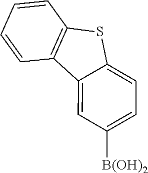 [SMILES: OB(O)c1ccc2sc3ccccc3c2c1] | 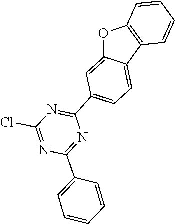 [SMILES: Clc1nc(-c2ccccc2)nc(-c2ccc3c(c2)oc2ccccc23)n1] | 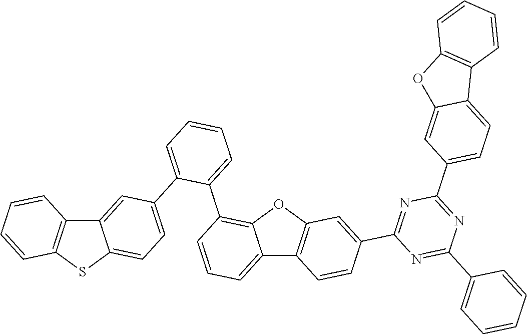 [SMILES: c1ccc(-c2nc(-c3ccc4c(c3)oc3ccccc34)nc(-c3ccc4c(c3)oc3c(-c5ccccc5-c5ccc6sc7ccccc7c6c5)cccc34)n2)cc1] | 69% |
| 354 | 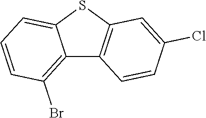 [SMILES: Clc1ccc2c(c1)sc1cccc(Br)c12] | 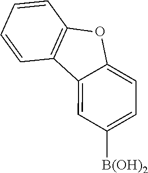 [SMILES: OB(O)c1ccc2oc3ccccc3c2c1] | 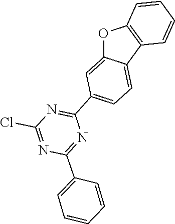 [SMILES: Clc1nc(-c2ccccc2)nc(-c2ccc3c(c2)oc2ccccc23)n1] | 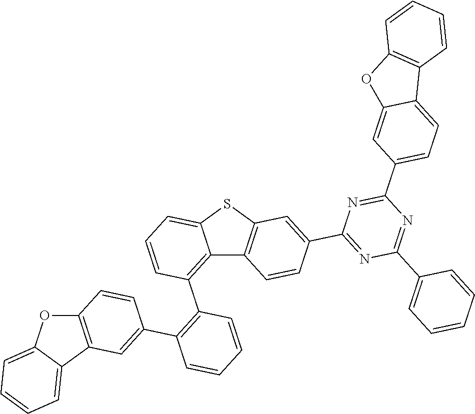 [SMILES: c1ccc(-c2nc(-c3ccc4c(c3)oc3ccccc34)nc(-c3ccc4c(c3)sc3cccc(-c5ccccc5-c5ccc6oc7ccccc7c6c5)c34)n2)cc1] | 71% |
| 360 | 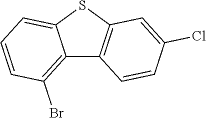 [SMILES: Clc1ccc2c(c1)sc1cccc(Br)c12] | 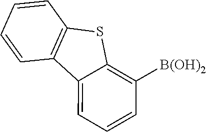 [SMILES: OB(O)c1cccc2c1sc1ccccc12] | 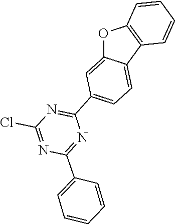 [SMILES: Clc1nc(-c2ccccc2)nc(-c2ccc3c(c2)oc2ccccc23)n1] | 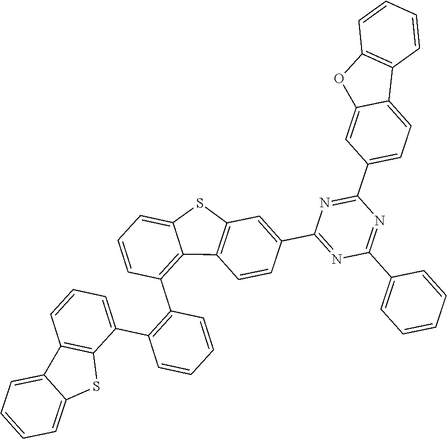 [SMILES: c1ccc(-c2nc(-c3ccc4c(c3)oc3ccccc34)nc(-c3ccc4c(c3)sc3cccc(-c5ccccc5-c5cccc6c5sc5ccccc56)c34)n2)cc1] | 72% |
| 368 | 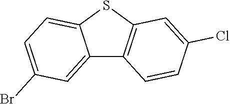 [SMILES: Clc1ccc2c(c1)sc1ccc(Br)cc12] | 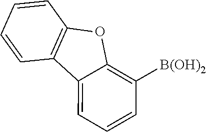 [SMILES: OB(O)c1cccc2c1oc1ccccc12] | 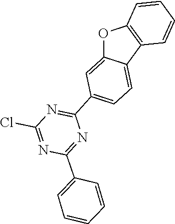 [SMILES: Clc1nc(-c2ccccc2)nc(-c2ccc3c(c2)oc2ccccc23)n1] | 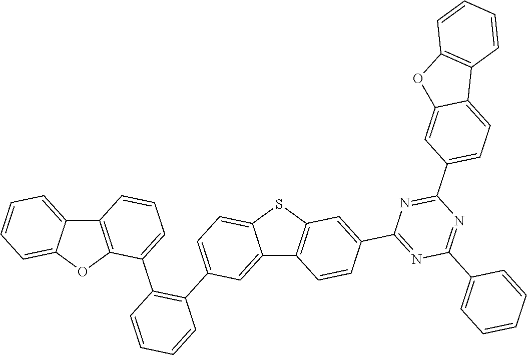 [SMILES: c1ccc(-c2nc(-c3ccc4c(c3)oc3ccccc34)nc(-c3ccc4c(c3)sc3ccc(-c5ccccc5-c5cccc6c5oc5ccccc56)cc34)n2)cc1] | 71% |
| 438 | 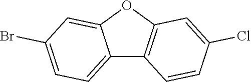 [SMILES: Clc1ccc2c(c1)oc1cc(Br)ccc12] | 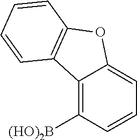 | 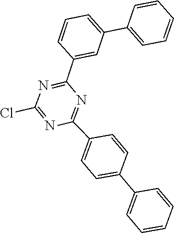 [SMILES: Clc1nc(-c2ccc(-c3ccccc3)cc2)nc(-c2cccc(-c3ccccc3)c2)n1] | 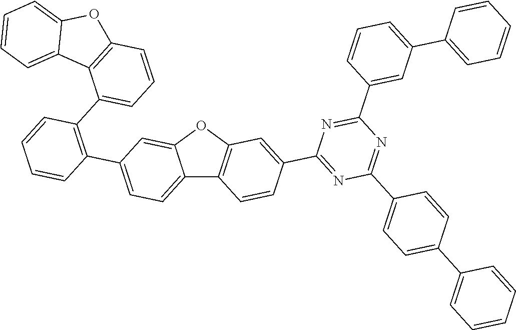 [SMILES: c1ccc(-c2ccc(-c3nc(-c4cccc(-c5ccccc5)c4)nc(-c4ccc5c(c4)oc4cc(-c6ccccc6-c6cccc7oc8ccccc8c67)ccc45)n3)cc2)cc1] | 69% |
| 468 | 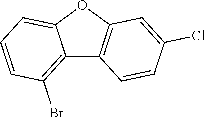 [SMILES: Clc1ccc2c(c1)oc1cccc(Br)c12] | 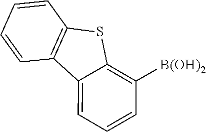 [SMILES: OB(O)c1cccc2c1sc1ccccc12] | 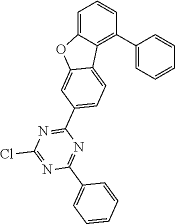 [SMILES: Clc1nc(-c2ccccc2)nc(-c2ccc3c(c2)oc2cccc(-c4ccccc4)c23)n1] | 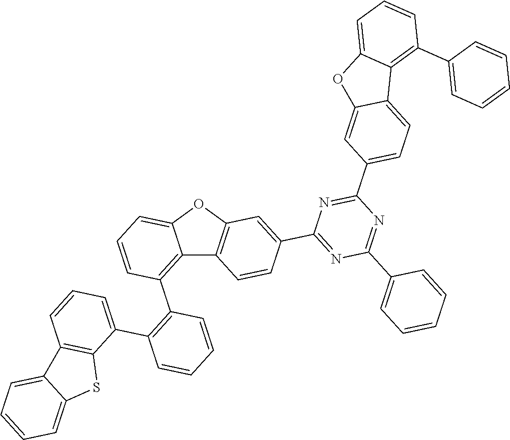 [SMILES: c1ccc(-c2nc(-c3ccc4c(c3)oc3cccc(-c5ccccc5)c34)nc(-c3ccc4c(c3)oc3cccc(-c5ccccc5-c5cccc6c5sc5ccccc56)c34)n2)cc1] | 67% |
| 511 | 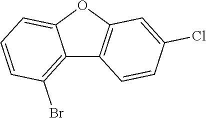 [SMILES: Clc1ccc2c(c1)oc1cccc(Br)c12] | 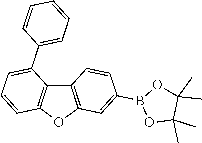 [SMILES: CC1(C)OB(c2ccc3c(c2)oc2cccc(-c4ccccc4)c23)OC1(C)C] | 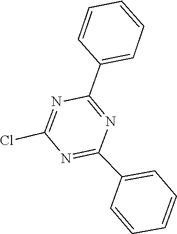 [SMILES: Clc1nc(-c2ccccc2)nc(-c2ccccc2)n1] | 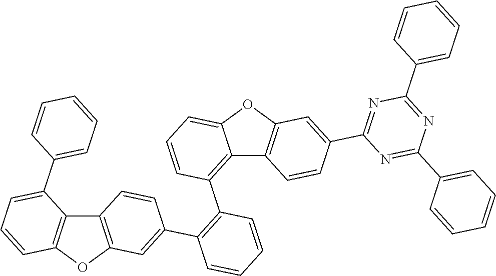 [SMILES: c1ccc(-c2nc(-c3ccccc3)nc(-c3ccc4c(c3)oc3cccc(-c5ccccc5-c5ccc6c(c5)oc5cccc(-c7ccccc7)c56)c34)n2)cc1] | 68% |
| 541 | 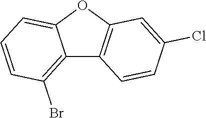 [SMILES: Clc1ccc2c(c1)oc1cccc(Br)c12] | 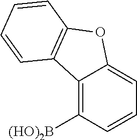 | 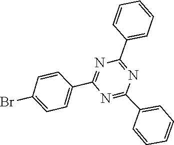 [SMILES: Brc1ccc(-c2nc(-c3ccccc3)nc(-c3ccccc3)n2)cc1] | 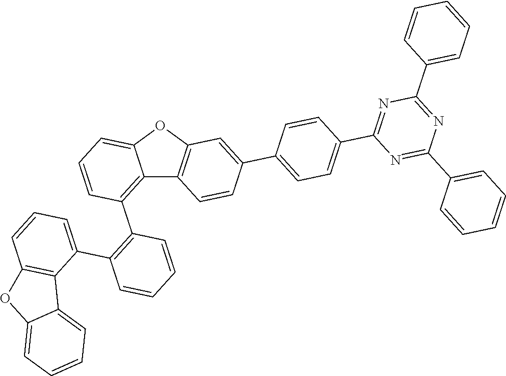 [SMILES: c1ccc(-c2nc(-c3ccccc3)nc(-c3ccc(-c4ccc5c(c4)oc4cccc(-c6ccccc6-c6cccc7oc8ccccc8c67)c45)cc3)n2)cc1] | 69% |
| 556 | 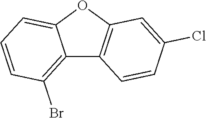 [SMILES: Clc1ccc2c(c1)oc1cccc(Br)c12] | 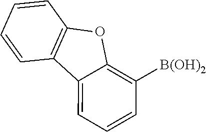 [SMILES: OB(O)c1cccc2c1oc1ccccc12] | 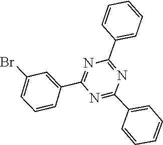 [SMILES: Brc1cccc(-c2nc(-c3ccccc3)nc(-c3ccccc3)n2)c1] | 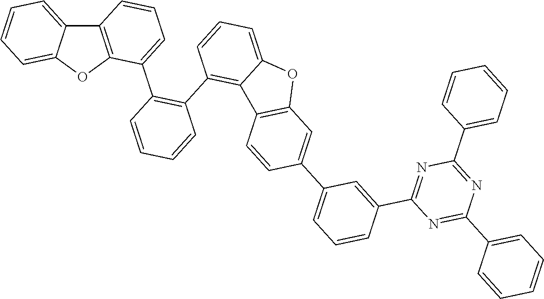 [SMILES: c1ccc(-c2nc(-c3ccccc3)nc(-c3cccc(-c4ccc5c(c4)oc4cccc(-c6ccccc6-c6cccc7c6oc6ccccc67)c45)c3)n2)cc1] | 70% |
| 569 | 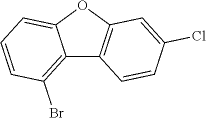 [SMILES: Clc1ccc2c(c1)oc1cccc(Br)c12] | 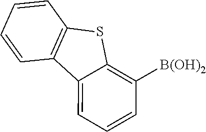 [SMILES: OB(O)c1cccc2c1sc1ccccc12] | 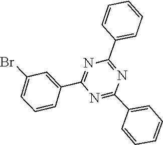 [SMILES: Brc1cccc(-c2nc(-c3ccccc3)nc(-c3ccccc3)n2)c1] | 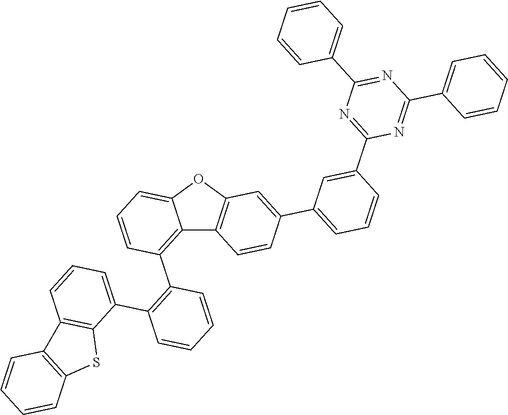 [SMILES: c1ccc(-c2nc(-c3ccccc3)nc(-c3cccc(-c4ccc5c(c4)oc4cccc(-c6ccccc6-c6cccc7c6sc6ccccc67)c45)c3)n2)cc1] | 71% |
| 572 | 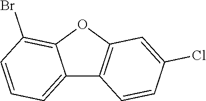 [SMILES: Clc1ccc2c(c1)oc1c(Br)cccc12] | 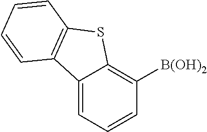 [SMILES: OB(O)c1cccc2c1sc1ccccc12] | 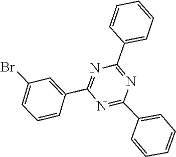 [SMILES: Brc1cccc(-c2nc(-c3ccccc3)nc(-c3ccccc3)n2)c1] | 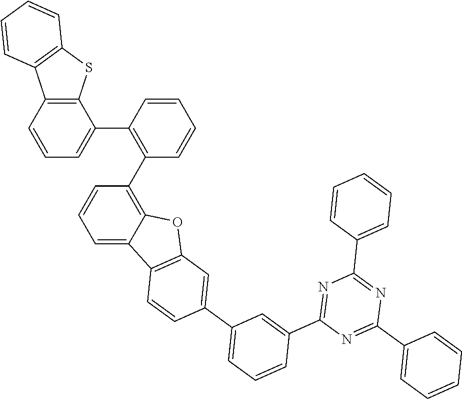 [SMILES: c1ccc(-c2nc(-c3ccccc3)nc(-c3cccc(-c4ccc5c(c4)oc4c(-c6ccccc6-c6cccc7c6sc6ccccc67)cccc45)c3)n2)cc1] | 72% |

### <Preparation Example 2> Preparation of Compound 49

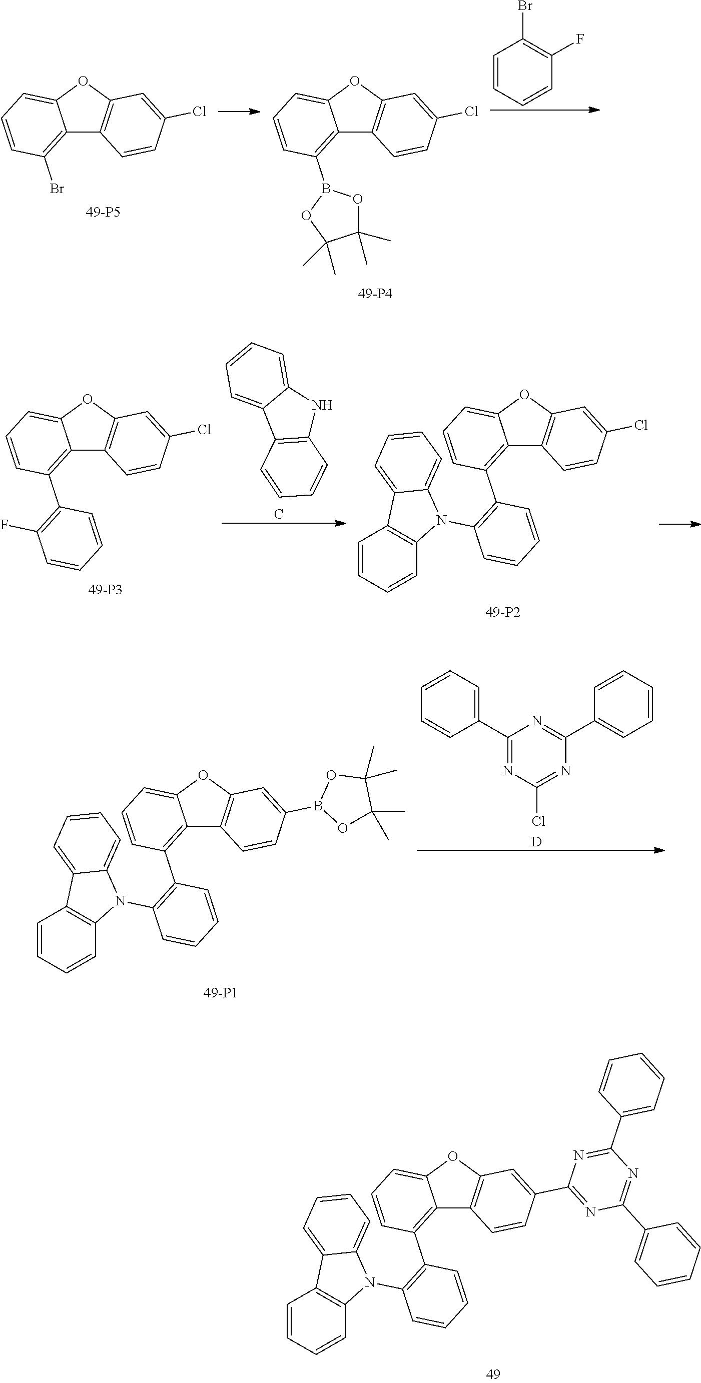

### 1) Preparation of Compound 49-P4

After 20 g (71.04 mmol) of Compound 49-P5 (1-bromo-7-chlorodibenzo[b,d]furan) and 27.06 g (106.56 mmol) of 4,4,4′,4′,5,5,5′,5′-octamethyl-2,2′-bi(1,3,2-dioxaborolane) were dissolved in 200 mL of 1,4-dioxane, 2.89 g (3.55 mmol) of Pd(dppf)Cl₂ and 20.92 g (213.12 mmol) of potassium acetate were added thereto, and the resulting mixture was stirred under reflux for 16 hours. After the reaction was completed, ethyl acetate was added to the reaction solution for dissolution, and then the resulting solution was extracted with distilled water, the organic layer was dried over anhydrous MgSO₄, and then the solvent was removed using a rotary evaporator, and then the residue was purified by column chromatography using dichloromethane and hexane as eluting solvents, thereby obtaining 18.5 g (yield 79%) of Compound 49-P4.

### 2) Preparation of Compound 49-P3

After 18.5 g (56.30 mmol) of Compound 49-P4 and 9.85 g (56.30 mmol) of 1-bromo-2-fluorobenzene were dissolved in 200 mL of 1,4-dioxane and 40 mL of distilled water, 3.25 g (2.81 mmol) of Pd(PPh₃)₄ and 19.45 g (140.75 mmol) of K₂CO₃ were added thereto, and the resulting mixture was stirred under reflux for 16 hours. After the reaction was completed, ethyl acetate was added to the reaction solution and dissolved, then the resulting solution was extracted with distilled water, the organic layer was dried over anhydrous MgSO₄, and then the solvent was removed using a rotary evaporator. Thereafter, purification was performed by column chromatography using dichloromethane and hexane as eluting solvents, thereby obtaining 12.7 g (yield 76%) of Compound 49-P3.

### 3) Preparation of Compound 49-P2

After 12.7 g (42.80 mmol) of Compound 49-P3 and 7.16 g (42.80 mmol) of 9H-carbazole (Compound C) were dissolved in 200 mL of N,N-dimethylacetamide, 41.84 g (128.40 mmol) of Cs₂CO₃ was added thereto, and the resulting mixture was stirred under reflux for 24 hours. After the reaction was completed, ethyl acetate was added to the reaction solution and dissolved, then the resulting solution was extracted with distilled water, the organic layer was dried over anhydrous MgSO₄, and then the solvent was removed using a rotary evaporator. Thereafter, purification was performed by column chromatography using dichloromethane and hexane as eluting solvents, thereby obtaining 13.5 g (yield 71%) of Compound 49-P2.

### 4) Preparation of Compound 49-P1

After 13.5 g (30.41 mmol) of Compound 49-P2 and 11.58 g (45.62 mmol) of 4,4,4′,4′,5,5,5′,5′-octamethyl-2,2′-bi(1,3,2-dioxaborolane) were dissolved in 135 mL of 1,4-dioxane, 1.39 g (1.52 mmol) of Pd₂(dba)₃, 8.95 g (91.23 mmol) of potassium acetate, and 1.45 g (3.04 mmol) of Xphos were added thereto, and the resulting mixture was stirred under reflux for 16 hours. After the reaction was completed, ethyl acetate was added to the reaction solution for dissolution, and then the resulting solution was extracted with distilled water, the organic layer was dried over anhydrous MgSO₄, and then the solvent was removed using a rotary evaporator, and then the residue was purified by column chromatography using dichloromethane and hexane as eluting solvents, thereby obtaining 12.4 g (yield 76%) of Compound 49-P1.

### 5) Preparation of Compound 49

After 12.4 g (23.16 mmol) of Compound 49-P1 and 6.20 g (23.16 mmol) of 2-chloro-4,6-diphenyl-1,3,5-triazine (Compound D) were dissolved in 150 mL of 1,4-dioxane and 30 mL of distilled water, 1.34 g (1.16 mmol) of Pd(PPh₃)₄ and 8.00 g (57.90 mmol) of K₂CO₃ were added thereto, and the resulting mixture was stirred under reflux for 16 hours. After the reaction was completed, ethyl acetate was added to the reaction solution and dissolved, then the resulting solution was extracted with distilled water, the organic layer was dried over anhydrous MgSO₄, and then the solvent was removed using a rotary evaporator. Thereafter, purification was performed by column chromatography using dichloromethane and hexane as eluting solvents, thereby obtaining 11.2 g (yield 75%) of Compound 49.

Target compounds in the following Table 2 were synthesized by performing the preparation in the same manner as in Preparation Example 2, except that Compound P5″ in the following Table 2 was used instead of Compound 49-P5, Compound C′ in the following Table 2 was used instead of Compound C, and Compound D′ in the following Table 2 was used instead of Compound D.

TABLE 2

| Com- | | | | | |
|---|---|---|---|---|---|
| pound | Compound | Compound | | Target | |
| No. | P5″ | C′ | Compound D′ | Compound | Yield |
| 50 | 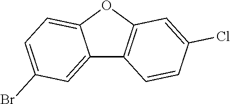 [SMILES: Clc1ccc2c(c1)oc1ccc(Br)cc12] | 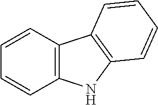 [SMILES: c1ccc2c(c1)[nH]c1ccccc12] | 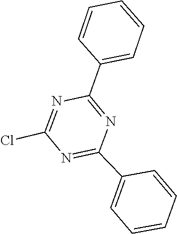 [SMILES: Clc1nc(-c2ccccc2)nc(-c2ccccc2)n1] | 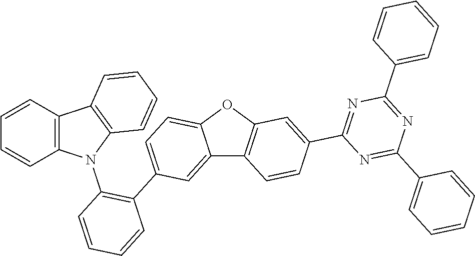 [SMILES: c1ccc(-c2nc(-c3ccccc3)nc(-c3ccc4c(c3)oc3ccc(-c5ccccc5-n5c6ccccc6c6ccccc65)cc34)n2)cc1] | 69% |
| 52 | 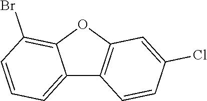 [SMILES: Clc1ccc2c(c1)oc1c(Br)cccc12] | 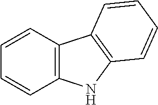 [SMILES: c1ccc2c(c1)[nH]c1ccccc12] | 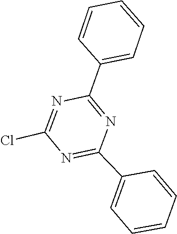 [SMILES: Clc1nc(-c2ccccc2)nc(-c2ccccc2)n1] | 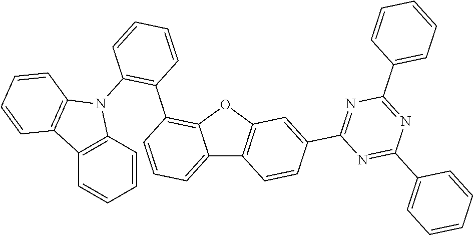 [SMILES: c1ccc(-c2nc(-c3ccccc3)nc(-c3ccc4c(c3)oc3c(-c5ccccc5-n5c6ccccc6c6ccccc65)cccc34)n2)cc1] | 71% |
| 152 | 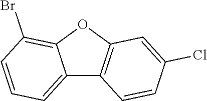 [SMILES: Clc1ccc2c(c1)oc1c(Br)cccc12] | 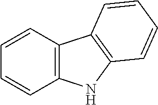 [SMILES: c1ccc2c(c1)[nH]c1ccccc12] | 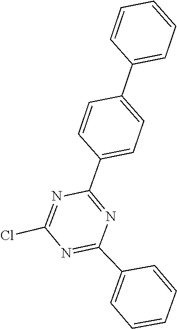 [SMILES: Clc1nc(-c2ccccc2)nc(-c2ccc(-c3ccccc3)cc2)n1] | 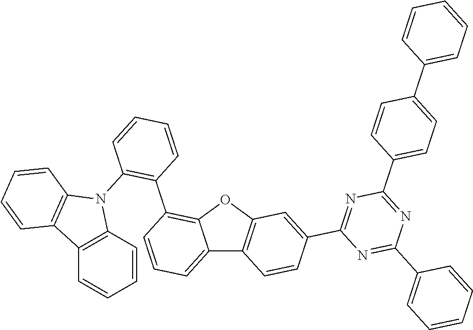 [SMILES: c1ccc(-c2ccc(-c3nc(-c4ccccc4)nc(-c4ccc5c(c4)oc4c(-c6ccccc6-n6c7ccccc7c7ccccc76)cccc45)n3)cc2)cc1] | 70% |
| 252 | 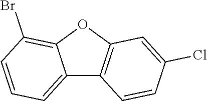 [SMILES: Clc1ccc2c(c1)oc1c(Br)cccc12] | 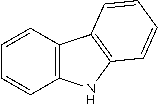 [SMILES: c1ccc2c(c1)[nH]c1ccccc12] | 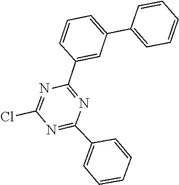 [SMILES: Clc1nc(-c2ccccc2)nc(-c2cccc(-c3ccccc3)c2)n1] | 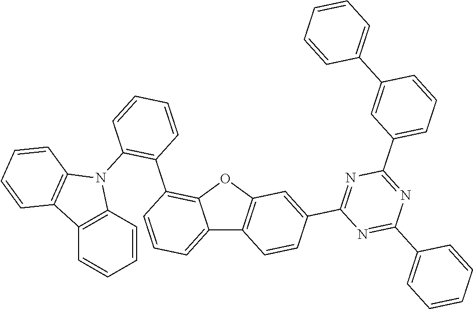 [SMILES: c1ccc(-c2cccc(-c3nc(-c4ccccc4)nc(-c4ccc5c(c4)oc4c(-c6ccccc6-n6c7ccccc7c7ccccc76)cccc45)n3)c2)cc1] | 71% |
| 352 | 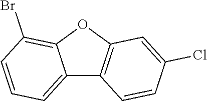 [SMILES: Clc1ccc2c(c1)oc1c(Br)cccc12] | 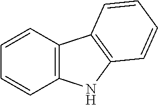 [SMILES: c1ccc2c(c1)[nH]c1ccccc12] | 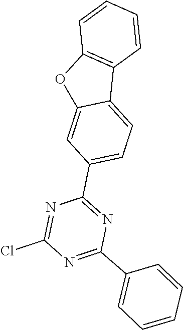 [SMILES: Clc1nc(-c2ccccc2)nc(-c2ccc3c(c2)oc2ccccc23)n1] | 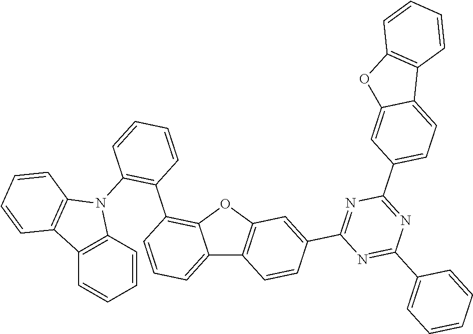 [SMILES: c1ccc(-c2nc(-c3ccc4c(c3)oc3ccccc34)nc(-c3ccc4c(c3)oc3c(-c5ccccc5-n5c6ccccc6c6ccccc65)cccc34)n2)cc1] | 72% |
| 401 | 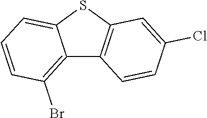 [SMILES: Clc1ccc2c(c1)sc1cccc(Br)c12] | 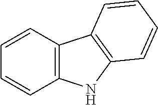 [SMILES: c1ccc2c(c1)[nH]c1ccccc12] | 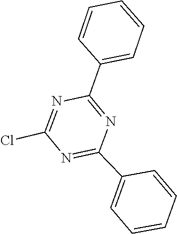 [SMILES: Clc1nc(-c2ccccc2)nc(-c2ccccc2)n1] | 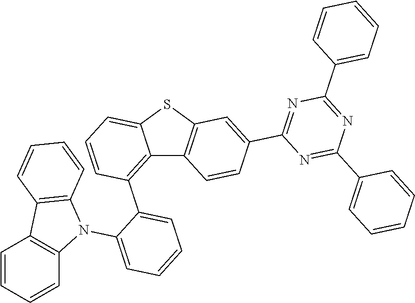 [SMILES: c1ccc(-c2nc(-c3ccccc3)nc(-c3ccc4c(c3)sc3cccc(-c5ccccc5-n5c6ccccc6c6ccccc65)c34)n2)cc1] | 67% |
| 409 | 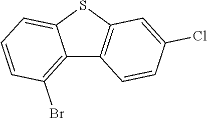 [SMILES: Clc1ccc2c(c1)sc1cccc(Br)c12] | 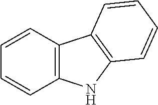 [SMILES: c1ccc2c(c1)[nH]c1ccccc12] | 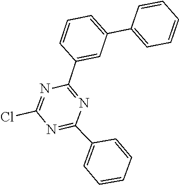 [SMILES: Clc1nc(-c2ccccc2)nc(-c2cccc(-c3ccccc3)c2)n1] | 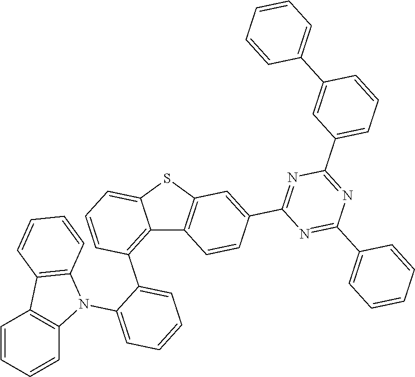 [SMILES: c1ccc(-c2cccc(-c3nc(-c4ccccc4)nc(-c4ccc5c(c4)sc4cccc(-c6ccccc6-n6c7ccccc7c7ccccc76)c45)n3)c2)cc1] | 68% |
| 413 | 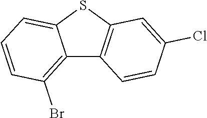 [SMILES: Clc1ccc2c(c1)sc1cccc(Br)c12] | 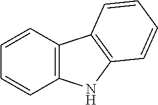 [SMILES: c1ccc2c(c1)[nH]c1ccccc12] | 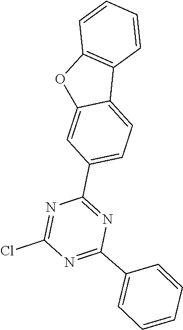 [SMILES: Clc1nc(-c2ccccc2)nc(-c2ccc3c(c2)oc2ccccc23)n1] | 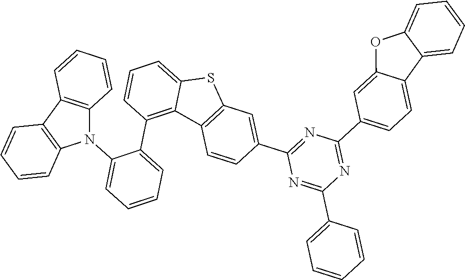 [SMILES: c1ccc(-c2nc(-c3ccc4c(c3)oc3ccccc34)nc(-c3ccc4c(c3)sc3cccc(-c5ccccc5-n5c6ccccc6c6ccccc65)c34)n2)cc1] | 69% |
| 417 | 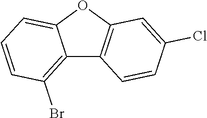 [SMILES: Clc1ccc2c(c1)oc1cccc(Br)c12] | 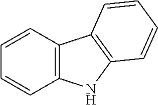 [SMILES: c1ccc2c(c1)[nH]c1ccccc12] | 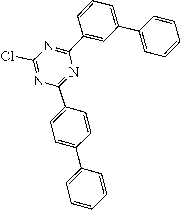 [SMILES: Clc1nc(-c2ccc(-c3ccccc3)cc2)nc(-c2cccc(-c3ccccc3)c2)n1] | 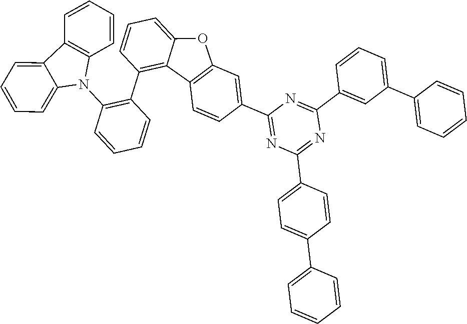 [SMILES: c1ccc(-c2ccc(-c3nc(-c4cccc(-c5ccccc5)c4)nc(-c4ccc5c(c4)oc4cccc(-c6ccccc6-n6c7ccccc7c7ccccc76)c45)n3)cc2)cc1] | 70% |
| 469 | 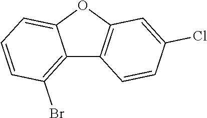 [SMILES: Clc1ccc2c(c1)oc1cccc(Br)c12] | 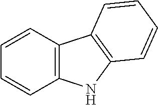 [SMILES: c1ccc2c(c1)[nH]c1ccccc12] | 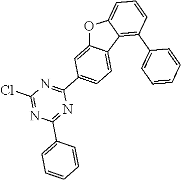 [SMILES: Clc1nc(-c2ccccc2)nc(-c2ccc3c(c2)oc2cccc(-c4ccccc4)c23)n1] | 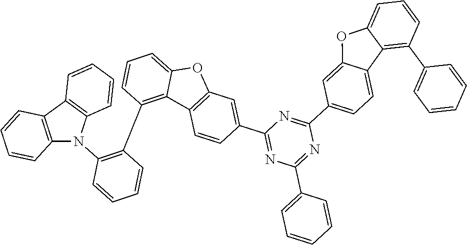 [SMILES: c1ccc(-c2nc(-c3ccc4c(c3)oc3cccc(-c5ccccc5)c34)nc(-c3ccc4c(c3)oc3cccc(-c5ccccc5-n5c6ccccc6c6ccccc65)c34)n2)cc1] | 71% |
| 492 | 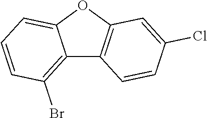 [SMILES: Clc1ccc2c(c1)oc1cccc(Br)c12] | 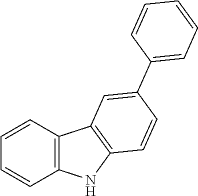 [SMILES: c1ccc(-c2ccc3[nH]c4ccccc4c3c2)cc1] | 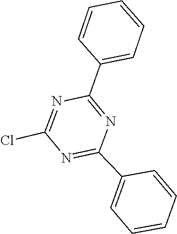 [SMILES: Clc1nc(-c2ccccc2)nc(-c2ccccc2)n1] | 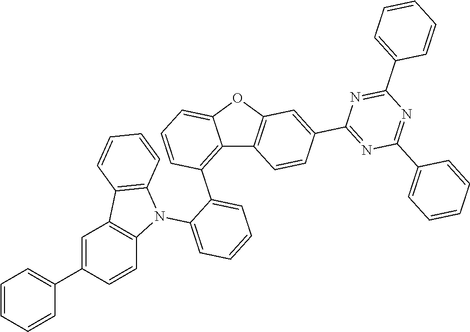 [SMILES: c1ccc(-c2ccc3c(c2)c2ccccc2n3-c2ccccc2-c2cccc3oc4cc(-c5nc(-c6ccccc6)nc(-c6ccccc6)n5)ccc4c23)cc1] | 72% |
| 534 | 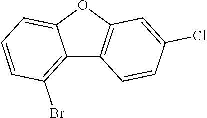 [SMILES: Clc1ccc2c(c1)oc1cccc(Br)c12] | 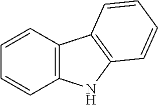 [SMILES: c1ccc2c(c1)[nH]c1ccccc12] | 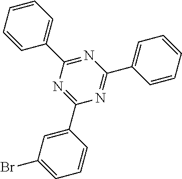 [SMILES: Brc1cccc(-c2nc(-c3ccccc3)nc(-c3ccccc3)n2)c1] | 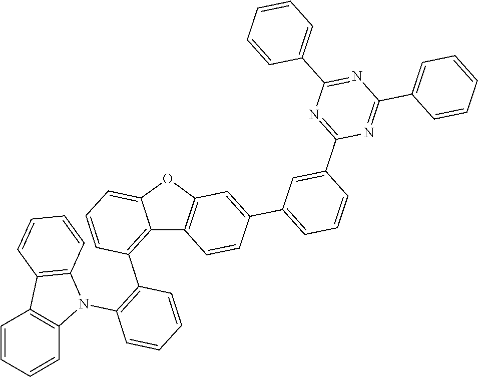 [SMILES: c1ccc(-c2nc(-c3ccccc3)nc(-c3cccc(-c4ccc5c(c4)oc4cccc(-c6ccccc6-n6c7ccccc7c7ccccc76)c45)c3)n2)cc1] | 71% |
| 596 | 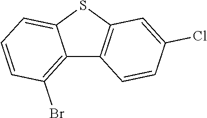 [SMILES: Clc1ccc2c(c1)sc1cccc(Br)c12] | 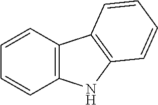 [SMILES: c1ccc2c(c1)[nH]c1ccccc12] | 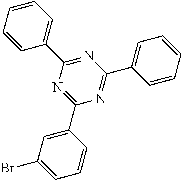 [SMILES: Brc1cccc(-c2nc(-c3ccccc3)nc(-c3ccccc3)n2)c1] | 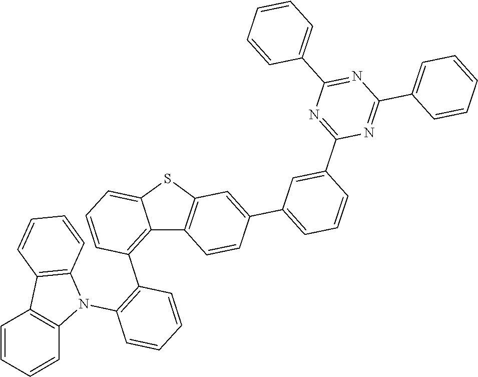 [SMILES: c1ccc(-c2nc(-c3ccccc3)nc(-c3cccc(-c4ccc5c(c4)sc4cccc(-c6ccccc6-n6c7ccccc7c7ccccc76)c45)c3)n2)cc1] | 70% |

### <Preparation Example 3> Preparation of Compound 2-79

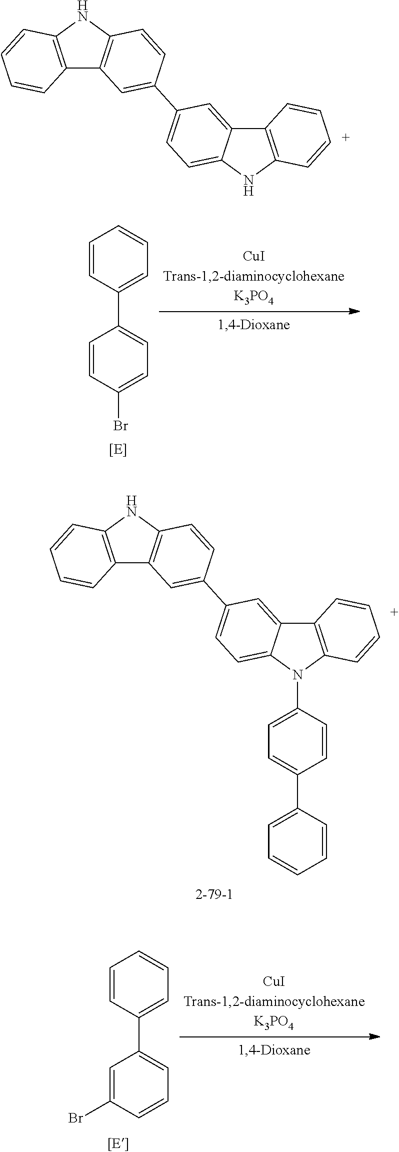 [SMILES: [C+]1=Cc2[nH]c3ccc(-c4ccc5[nH]c6ccccc6c5c4)cc3c2C=C1 ; Brc1ccc(-c2ccccc2)cc1 ; c1ccc(-c2ccc(-n3c4ccccc4c4cc(-c5ccc6[nH]c7ccccc7c6c5)ccc43)cc2)cc1 ; Brc1cccc(-c2ccccc2)c1]

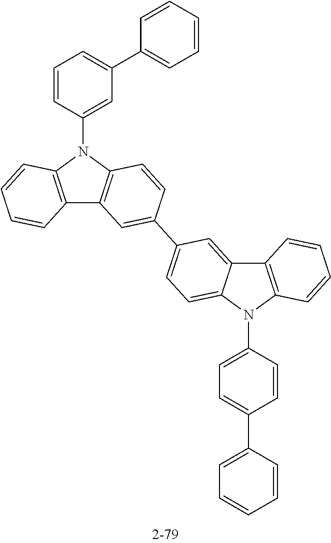 [SMILES: c1ccc(-c2ccc(-n3c4ccccc4c4cc(-c5ccc6c(c5)c5ccccc5n6-c5cccc(-c6ccccc6)c5)ccc43)cc2)cc1]

### 1) Preparation of Compound 2-79-1

After 9H,9′H-3,3′-bicarbazole (10 g, 0.030 mol), (4-bromo-1,1′-biphenyl-) (Compound E) (7.26 g, 0.030 mol), CuI (0.57 g, 0.003 mol), trans-1,2-diaminocyclohexane (0.34 g, 0.003 mol), and K₃PO₄ (12.74 g, 0.06 mol) were dissolved in 100 mL of 1,4-dioxane in a one-neck round bottom flask, the resulting solution was refluxed at 125° C. for 8 hours. After the reaction was completed, distilled water and dichloromethane were added thereto at room temperature, extraction was performed, the organic layer was dried over MgSO₄, and then the solvent was removed by a rotary evaporator. The reaction product was purified by column chromatography (dichloromethane:hexane=1:3) and recrystallized with methanol to obtain Compound 2-79-1. (13.92 g, yield 94%)

### 2) Preparation of Compound 2-79

After Compound 2-79-1 (13.92 g, 0.028 mol), 3-bromo-1,1′-biphenyl (Compound E′) (6.83 g, 0.028 mol), CuI (0.53 g, 0.0028 mol), trans-1,2-diaminocyclohexane (0.32 g, 0.0028 mol), and K₃PO₄ (11.89 g, 0.056 mol) were dissolved in 140 mL of 1,4-dioxane in a one-neck round bottom flask, the resulting solution was refluxed at 125° C. for 8 hours. After the reaction was completed, distilled water and dichloromethane were added thereto at room temperature, extraction was performed, the organic layer was dried over MgSO₄, and then the solvent was removed by a rotary evaporator. The reaction product was purified by column chromatography (dichloromethane:hexane=1:3) and recrystallized with methanol to obtain Target Compound 2-79. (16.14 g, yield 88%)

In Preparation Example 3, when Compound E and Compound E′ are the same, the target compound may be immediately synthesized by adding 2 equivalents of Compound E in 1) of Preparation Example 3 above. That is, when Compound E and Compound E′ are the same, the aforementioned 2) of Preparation Example 3 may be omitted.

Target compounds in the following Table 3 were synthesized by performing the preparation in the same manner as in Preparation Example 3, except that Compound El in the following Table 3 was used instead of Compound E, and Compound E′1 in the following Table 3 was used instead of Compound E′.

TABLE 3

| Compound | Compound | Compound | Target | |
|---|---|---|---|---|
| No. | E1 | E′1 | Compound | Yield |
| 2-74 | 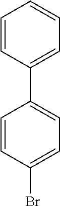 | 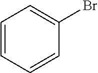 | 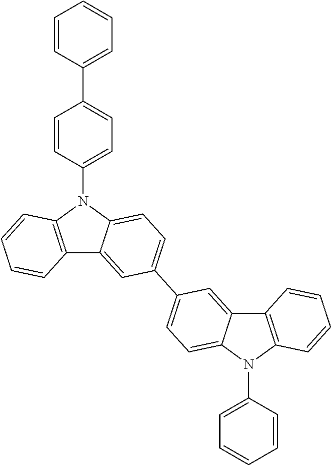 [SMILES: c1ccc(-c2ccc(-n3c4ccccc4c4cc(-c5ccc6c(c5)c5ccccc5n6-c5ccccc5)ccc43)cc2)cc1] | 73% |
| 2-76 | 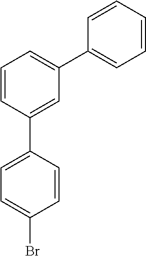 | 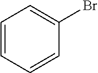 | 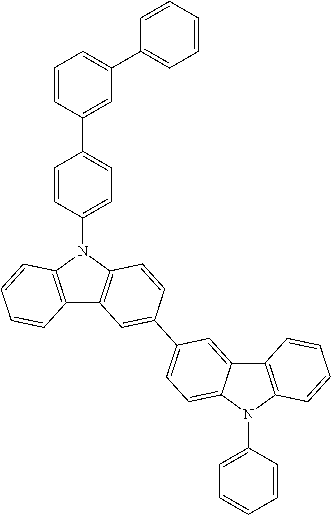 [SMILES: c1ccc(-c2cccc(-c3ccc(-n4c5ccccc5c5cc(-c6ccc7c(c6)c6ccccc6n7-c6ccccc6)ccc54)cc3)c2)cc1] | 72% |
| 2-77 | 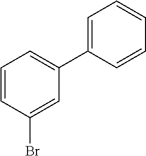 | 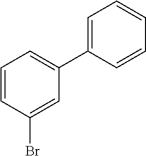 | 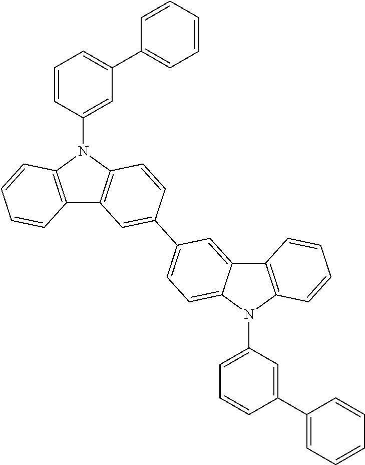 [SMILES: c1ccc(-c2cccc(-n3c4ccccc4c4cc(-c5ccc6c(c5)c5ccccc5n6-c5cccc(-c6ccccc6)c5)ccc43)c2)cc1] | 83% |
| 2-78 | 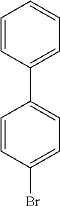 | 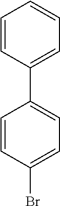 | 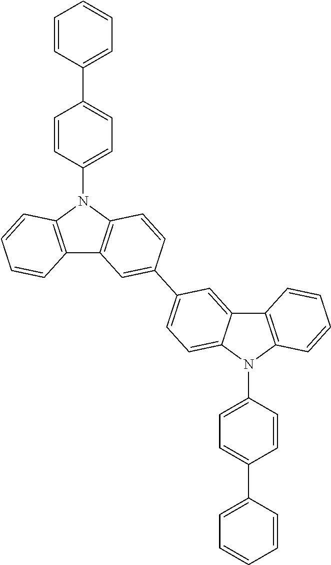 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | 88% |

### <Preparation Example 4> Preparation of Compound 2-57

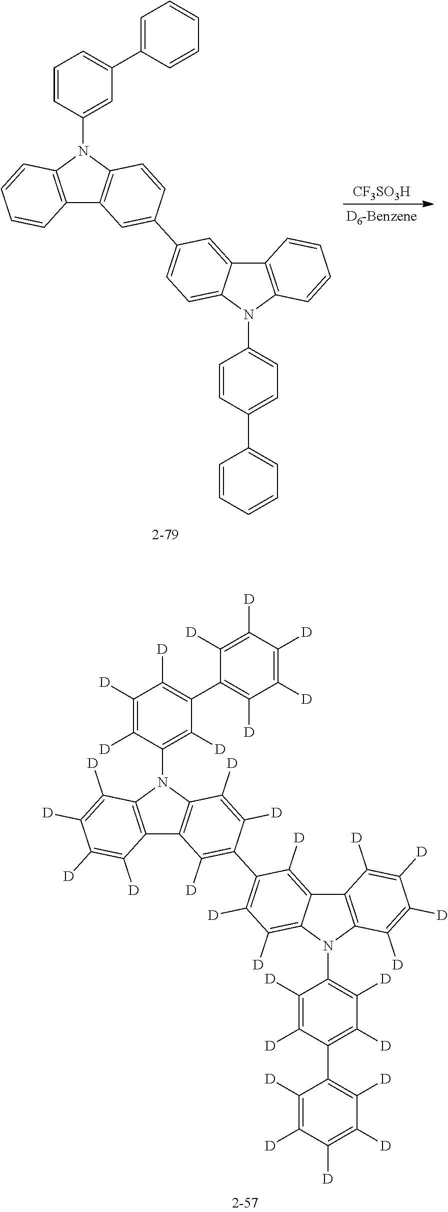 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; "CF3SO3H" ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c([2H])c2[2H])c([2H])c1[2H]]

### 1) Preparation of Compound 2-57

A mixture of Compound 2-79 (12.17 g, 0.017 mol) prepared in Preparation Example 3 above, 51.5 g of triflic acid and D₆-benzene (608.5 mL) was put into a one-neck round bottom flask and stirred at 50° C. for 1 hour. After the reaction was completed, the reaction product was quenched with Na₂CO₃ in D₂O. After quenching, dichloromethane was added to the mixed solution and dissolved, then the organic layer was separated and dried over anhydrous MgSO₄, and then the solvent was removed using a rotary evaporator. Thereafter, purification was performed by column chromatography using dichloromethane and hexane as eluting solvents, thereby obtaining Target Compound 2-57. (8.01 g, yield 70%)

Target compounds in the following Table 4 were synthesized by performing the preparation in the same manner as in Preparation Example 4, except that Compound E3 in the following Table 4 was used instead of Compound 2-79.

TABLE 4

| Compound | | | |
|---|---|---|---|
| No. | Compound E3 | Target Compound | Yield |
| 2-50 | 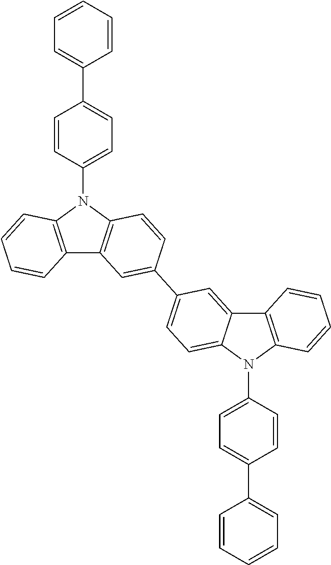 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | 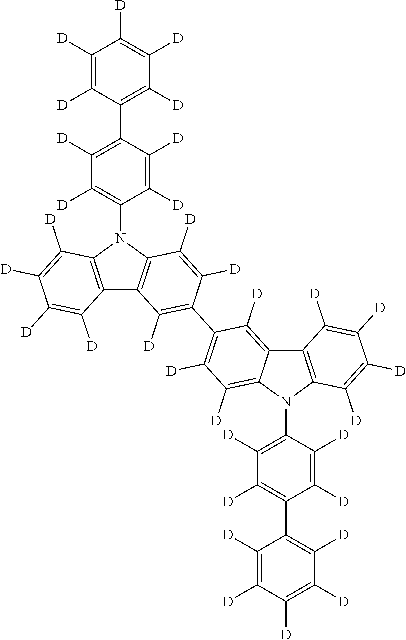 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]] | 68% |
| 2-51 | 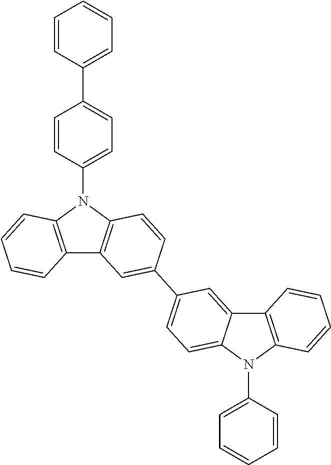 [SMILES: c1ccc(-c2ccc(-n3c4ccccc4c4cc(-c5ccc6c(c5)c5ccccc5n6-c5ccccc5)ccc43)cc2)cc1] | 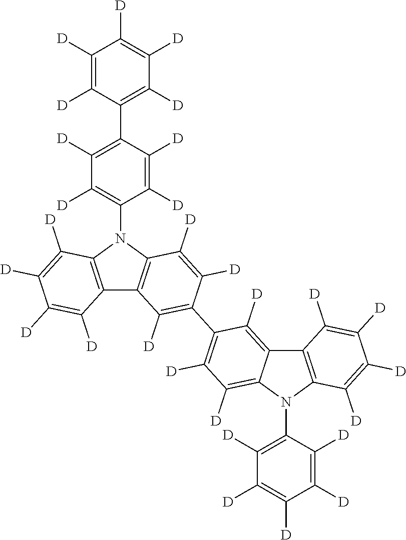 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]] | 68% |
| 2-53 | 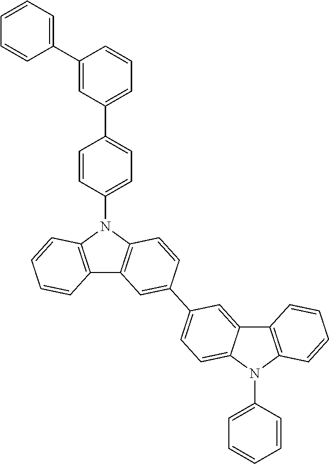 [SMILES: c1ccc(-c2cccc(-c3ccc(-n4c5ccccc5c5cc(-c6ccc7c(c6)c6ccccc6n7-c6ccccc6)ccc54)cc3)c2)cc1] | 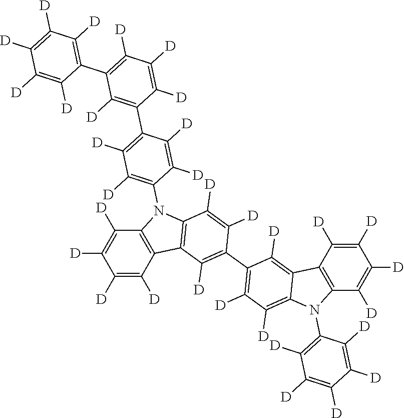 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3[2H])c2[2H])c([2H])c1[2H]] | 70% |
| 2-56 | 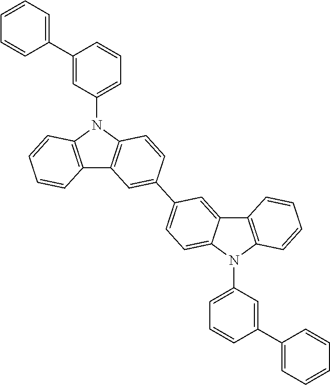 [SMILES: c1ccc(-c2cccc(-n3c4ccccc4c4cc(-c5ccc6c(c5)c5ccccc5n6-c5cccc(-c6ccccc6)c5)ccc43)c2)cc1] | 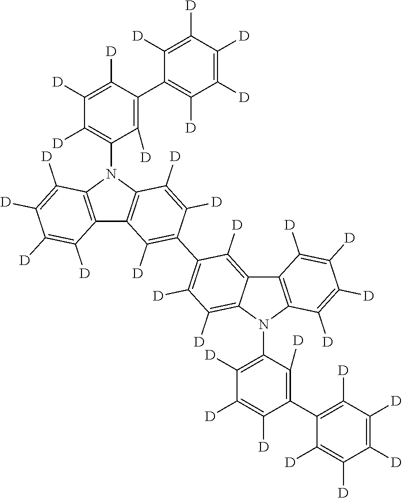 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c2[2H])c([2H])c1[2H]] | 69% |

The other compounds other than the compounds described in Preparation Examples 1 to 4 and Tables 1 to 4 were also prepared in the same manner as in the above-described Preparation Examples, and synthesis results are shown in the following Tables 5 and 6. The following Table 5 shows the measured values of ¹H NMR (CDCl₃, 400 Mz), and Table 6 shows the measured values of field desorption mass spectrometry (FD-MS).

TABLE 5

| Com- | |
|---|---|
| pound | ¹H NMR (CDCl₃, 400 MHZ) |
| 1 | δ = 8.36 (4H, d), 8.03-7.96 (4H, m), 7.82-7.76 (4H, m), 7.69 |
| | (2H, m), 7.60-7.50 (11H, m), 7.39-7.31 (2H, m) |
| 3 | δ = 8.36 (4H, d), 8.03-7.96 (5H, m), 7.82-7.76 (5H, m), 7.60- |
| | 7.50 (10H, m), 7.39-7.31 (2H, m) |
| 5 | δ = 8.45 (1H, d), 8.36 (4H, d), 8.03-7.93 (6H, m), 7.82-7.76 |
| | (3H, m), 7.69-7.49 (13H, m) |
| 8 | δ = 8.55 (1H, d), 8.45 (1H, d), 8.36-8.32 (5H, m), 8.03-7.93 |
| | (4H, m), 7.82-7.69 (5H, m), 7.60-7.49 (11H, m) |
| 10 | δ = 8.55 (1H, d), 8.36 (4H, d), 8.03-7.89 (6H, m), 7.82-7.76 |
| | (4H, m), 7.69-7.50 (15H, m), 7.35 (1H, t), 7.16 (1H, t) |
| 13 | δ = 8.36 (4H, d), 7.96-7.76 (10H, m), 7.69 (1H, d), 7.60- |
| | 7.50 (10H, m), 7.39-7.31 (2H, m) |
| 16 | δ = 8.36 (4H, d), 8.03-7.76 (11H, m), 7.60-7.50 (10H, m), |
| | 7.39-7.31 (2H, m) |
| 20 | δ = 8.55 (1H, d), 8.45 (1H, d), 8.36-8.32 (3H, m), 8.03-7.70 |
| | (10H, m), 7.60-7.49 (10H, m), |
| 24 | δ = 8.55 (1H, d), 8.36-8.29 (5H, m), 8.06-7.76 (10H, m), |
| | 7.62-7.48 (14H, m), 7.35 (1H, t), 7.16 (1H, t) |
| 25 | δ = 8.36 (4H, d), 8.03-7.96 (5H, m), 7.82-7.76 (5H, m), 7.69 |
| | (1H, d), 7.60-7.54 (10H, m), 7.39-7.31 (2H, m) |
| 32 | δ = 8.55 (1H, d), 8.45 (1H, d), 8.36-8.32 (5H, m), 8.03-7.93 |
| | (5H, m), 7.82-7.70 (5H, m), 7.60-7.49 (10H, m) |
| 34 | δ = 8.36 (4H, d), 8.08-7.76 (11H, m), 7.60-7.50 (10H, m), |
| | 7.39-7.31 (2H, m) |
| 40 | δ = 8.55 (1H, d), 8.45 (1H, d), 8.36-8.32 (5H, m), 8.08-7.93 |
| | (6H, m), 7.82-7.70 (3H, m), 7.60-7.49 (11H, m) |
| 49 | δ = 8.55 (1H, d), 8.36 (4H, d), 8.19 (1H, d), 8.03 (1H, d), |
| | 7.94-7.91 (3H, m), 7.82-7.76 (4H, m), 7.69 (1H, d), 7.58- |
| | 7.46 (10H, m), 7.35 (1H, t), 7.20 (1H, t) |
| 50 | δ = 8.55 (1H, d), 8.36 (4H, d), 8.19 (1H, d), 8.03 (1H, d), |
| | 7.94-7.76 (9H, m), 7.58-7.46 (9H, m), 7.35 (1H, t), 7.20- |
| | 7.16 (2H, m) |
| 52 | δ = 8.55 (1H, d), 8.36 (4H, d), 8.19 (1H, d), 8.08-8.02 (3H, |
| | m), 7.92-7.91 (2H, m), 7.82-7.76 (3H, m), 7.58-7.46 (10H, |
| | m), 7.35 (1H, t), 7.20-7.16 (2H, m) |
| 53 | δ = 8.36 (4H, d), 8.24-8.20 (2H, m), 8.03-7.94 (6H, m), 7.82 |
| | (1H, d), 7.69-7.50 (12H, m) |
| 56 | δ = 8.36 (4H, d), 8.24-8.20 (2H, m), 8.08-7.94 (8H, m), 7.68- |
| | 7.50 (11H, m), 7.39-7.31 (2H, m) |
| 57 | δ = 8.45 (1H, d), 8.36 (4H, d), 8.24-8.20 (2H, m), 8.03-7.93 |
| | (8H, m), 7.68-7.49 (12H, m) |
| 60 | δ = 8.55 (1H, d), 8.45 (1H, d), 8.36-8.32 (5H, m), 8.24-8.20 |
| | (2H, m), 8.03-7.93 (6H, m), 7.70-7.49 (12H, m) |
| 64 | δ = 8.55 (1H, d), 8.36-8.20 (7H, m), 8.06-7.94 (7H, m), 7.68- |
| | 7.48 (15H, m), 7.35 (1H, t), 7.16 (1H, t) |
| 101 | δ = 8.36 (2H, d), 8.03-7.96 (6H, m), 7.82-7.25 (23H, m) |
| 115 | δ = 8.36 (2H, d), 8.03-7.75 (16H, m), 7.60-7.25 (13H, m) |
| 132 | δ = 8.55 (1H, d), 8.45 (1H, d), 8.36-8.32 (3H, m), 8.03-7.93 |
| | (7H, m), 7.82-7.70 (7H, m), 7.60-7.41 (10H, m), 7.25 (2H, d) |
| 136 | δ = 8.36 (2H, d), 8.08-7.96 (10H, m), 7.82-7.75 (4H, m), |
| | 7.60-7.31 (15H, m) |
| 152 | δ = 8.55 (1H, d), 8.36 (2H, d), 8.19 (1H, d), 8.08-7.91 (8H, |
| | m), 7.82-7.75 (5H, m), 7.58-7.35 (11H, m), 7.25-7.16 (4H, m) |
| 153 | δ = 8.36 (2H, d), 8.24-8.20 (2H, m), 8.03-7.94 (8H, m), 7.82- |
| | 7.25 (19H, m) |
| 160 | δ = 8.55 (1H, d), 8.45 (1H, d), 8.36-8.32 (3H, m), 8.24-8.20 |
| | (2H, m), 8.03-7.93 (8H, m), 7.75-7.41 (14H, m), 7.25 (1H, |
| | d) |
| 165 | δ = 8.36 (2H, d), 8.24-8.20 (2H, m), 8.12 (2H, d), 7.99-7.94 |
| | (7H, m), 7.82-7.69 (4H, m), 7.60-7.31 (14H, m) |
| 180 | δ = 8.36 (2H, d), 8.24-8.17 (5H, m), 8.08-7.94 (8H, m), 7.75 |
| | (2H, d), 7.60-7.25 (14H, m) |
| 203 | δ = 8.38-8.36 (3H, m), 8.03-7.94 (6H, m), 7.82-7.31 (22H, |
| | m), |
| 220 | δ = 8.55 (1H, d), 8.45-8.32 (5H, m), 7.96-7.70 (14H, m), |
| | 7.60-7.41 (11H, m) |
| 226 | δ = 8.38-8.36 (3H, m), 8.03-7.73 (16H, m), 7.61-7.31 (12H, |
| | m) |
| 252 | δ = 8.55 (1H, d), 8.38-8.36 (3H, m), 8.19 (1H, d), 8.08-8.02 |
| | (3H, m), 7.94-7.91 (4H, m), 7.82-7.73 (6H, m), 7.61-7.35 |
| | (12H, m), 7.20-7.16 (2H, m) |
| 254 | δ = 8.38-8.36 (3H, m), 8.24-8.20 (2H, m), 8.03-7.31 (26H, m) |
| 301 | δ = 8.36 (2H, d), 8.03-7.96 (6H, m), 7.82-7.50 (17H, m), |
| | 7.39-7.31 (4H, m) |
| 313 | δ = 8.36 (2H, d), 8.03-7.69 (15H, m), 7.60-7.50 (8H, m), |
| | 7.39-7.31 (4H, m) |
| 327 | δ = 8.36 (2H, d), 8.03-7.96 (8H, m), 7.82-7.76 (8H, m), 7.60- |
| | 7.50 (7H, m), 7.39-7.31 (4H, m) |
| 338 | δ = 8.45 (1H, d), 8.36 (2H, d), 8.12-7.93 (11H, m), 7.82- |
| | 7.76 (4H, m), 7.60-7.49 (9H, m), 7.39-7.31 (2H, d) |
| 352 | δ = 8.55 (1H, d), 8.36 (2H, d), 8.19 (1H, d), 8.08-7.91 (8H, |
| | m), 7.82-7.76 (5H, m), 7.58-7.31 (11H, m), 7.20-7.16 (2H, |
| | m) |
| 354 | δ = 8.36 (2H, d), 8.24-8.20 (2H, m), 8.03-7. 76 (13H, m), |
| | 7.68-7.50 (8H, m), 7.39-7.31 (4H, m) |
| 360 | δ = 8.55 (1H, d), 8.45 (1H, d), 8.36-8.32 (3H, m), 8.24-8.20 |
| | (2H, m), 8.03-7.93 (8H, m), 7.82-7.31 (14H, m) |
| 368 | δ = 8.36 (2H, d), 8.24-8.20 (2H, m), 8.12-7.94 (11H, m), |
| | 7.82-7.76 (2H, m), 7.60-7.50 (8H, m), 7.39-7.31 (4H, m) |
| 401 | δ = 8.55 (1H, d), 8.36 (4H, d), 8.24-8.19 (3H, m), 8.03 (1H, |
| | d), 7.94-7.91 (5H, m), 7.80 (1H, t), 7.68 (1H, t), 7.58- |
| | 7.46 (9H, m), 7.35 (1H, t), 7.20-7.16 (2H, m) |
| 409 | δ = 8.55 (1H, d), 8.38-8.36 (3H, m), 8.24-8.19 (3H, m), 8.03 |
| | (1H, d), 7.94-7.91 (6H, m), 7.80-7.35 (13H, m), 7.20-7.16 |
| | (2H, m) |
| 413 | δ = 8.55 (1H, d), 8.36 (2H, d), 8.24-8.19 (3H, m), 8.03-7.91 |
| | (8H. m), 7.82-7.76 (3H, m), 7.68 (1H, t), 7.58-7.31 (10H, |
| | m), 7.20-7.16 (2H, m) |
| 417 | δ = 8.55 (1H, d), 8.38 (1H, d), 8.19 (1H, d), 8.03-7.91 (7H, |
| | m), 7.82-7.69 (10H, m), 7.69-7.35 (12H, m), 7.25-7.16 (4H, |
| | m) |
| 438 | δ = 8.38 (1H, d), 8.03-7.94 (7H, m), 7.82-7.69 (11H, m), |
| | 7.61-7.25 (15H, m) |
| 468 | δ = 8.55 (1H, d), 8.45 (1H, d), 8.36-8.32 (3H, m), 8.03-7.93 |
| | (5H, m), 7.82-7.41 (23H, m) |
| 469 | δ = 8.55 (1H, d), 8.36 (2H, d), 8.19 (1H, d), 8.03 (2H, d), |
| | 7.94-7.69 (14H, m), 7.58-7.35 (12H, m), 7.20-7.16 (2H, m) |
| 492 | δ = 8.55 (1H, d), 8.36 (4H, d), 8.03-7.89 (6H, m), 7.82-7.69 |
| | (8H, m), 7.57-7.35 (12H, m), 7.16 (1H, t) |
| 511 | δ = 8.36 (4H, d), 8.03-7.96 (4H, m), 7.82-7.69 (10H, m), |
| | 7.60-7.41 (13H, m) |
| 534 | δ = 8.55 (1H, d), 8.38-8.36 (5H, m), 8.19 (1H, d), 8.03 (1H, |
| | d), 7.94-7.91 (4H, m), 7.82-7.69 (6H, m), 7.61-7.46 (11H, |
| | m), 7.35 (1H, t), 7.20-7.16 (2H, m) |
| 541 | δ = 8.36 (4H, d), 8.03-7.96 (6H, m), 7.82-7.76 (4H, m), 7.69 |
| | (2H, d), 7.60-7.50 (11H, m), 7.39-7.25 (4H, m) |
| 556 | δ = 8.38-8.36 (5H, m), 8.08-7.94 (7H, m), 7.82-7.69 (5H, m), |
| | 7.61-7.50 (12H, m), 7.39-7.31 (2H, m) |
| 569 | δ = 8.55 (1H, d), 8.45-8.32 (7H, m), 8.03-7.93 (5H, m), 7.82- |
| | 7.70 (6H, m), 7.61-7.49 (12H, m) |
| 572 | δ = 8.55 (1H, d), 8.45-8.32 (7H, m), 8.03-7.93 (7H, m), 7.82- |
| | 7.70 (4H, m), 7.60-7.49 (12H, m) |
| 596 | δ = 8.55 (1H, d), 8.38-8.36 (5H, m), 8.24-8.17 (4H, m), 8.03 |
| | (1H, d), 7.94-7.91 (5H, m), 7.94-7.46 (13H, m), 7.35 (1H, |
| | t), 7.20-7.16 (2H, m) |
| 2-50 | δ = deuterium content of 100% with no ¹H NMR peak |
| 2-51 | δ = deuterium content of 100% with no ¹H NMR peak |
| 2-53 | δ = deuterium content of 100% with no ¹H NMR peak |
| 2-56 | δ = deuterium content of 100% with no ¹H NMR peak |
| 2-57 | δ = deuterium content of 100% with no ¹H NMR peak |
| 2-74 | δ = 8.55 (1H, d), 8.30 (1H, d), 8.19-8.13 (2H, m), 7.94-7.89 |
| | (8H, m), 7.77-7.75 (3H, m), 7.62-7.35 (11H, m), 7.20-7.16 |
| | (2H m) |
| 2-76 | δ = 8.55 (1H, d), 8.30 (1H, d), 8.13~8.19 (2H, m), 7.89~7.99 |
| | (9H, m), 7.73~7.77 (4H, m), 7.35~7.62 (13H, m), 7.16~7.20 |
| | (2H, m) |
| 2-77 | δ = 8.55 (1H, d), 8.30 (1H, d), 8.13~8.21 (4H, m), 7.89~7.99 |
| | (4H, m), 7.35~7.77 (20H, m), 7.16~7.20 (2H, t) |
| 2-78 | δ = 8.55 (1H, d), 8.30 (1H, d), 8.13~8.19 (2H, m), 7.89~7.99 |
| | (12H, m), 7.75~7.77 (5H, m), 7.58 (1H, d), 7.35~7.50 (8H, |
| | m), 7.16~7.20 (2H, m) |
| 2-79 | δ = 8.55 (1H, d), 8.30 (1H, d), 8.21-8.13 (3H, m), 7.99-7.89 |
| | (8H, m), 7.77-7.35 (12H, m), 7.20-7.16 (2H, m) |

TABLE 6

| Compound | FD-MS | Compound | FD-MS |
|---|---|---|---|
| 1 | m/z = | 3 | m/z = |
| | 641.21 (C₄₅H₂₇N₃O₂ = 641.73) | | 641.21 (C₄₅H₂₇N₃O₂ = 641.73) |
| 5 | m/z = | 8 | m/z = |
| | 657.19 (C₄₅H₂₇N₃OS = 657. 79) | | 657. 19 (C₄₅H₂₇N₃OS = 657. 79) |
| 10 | m/z = | 13 | m/z = |
| | 716.26 (C₅₁H₃₂N₄O = 716.84) | | 641.21 (C₄₅H₂₇N₃O₂ = 641.73) |
| 16 | m/z = | 20 | m/z = |
| | 641.21 (C₄₅H₂₇N₃O₂ = 641.73) | | 657.19 (C₄₅H₂₇N₃OS = 657. 79) |
| 24 | m/z = | 25 | m/z = |
| | 716.26 (C₅₁H₃₂N₄O = 716.84) | | 641.21 (C₄₅H₂₇N₃O₂ = 641.73) |
| 32 | m/z = | 34 | m/z = |
| | 657.19 (C₄₅H₂₇N₃OS = 657. 79) | | 641.21 (C₄₅H₂₇N₃O₂ = 641.73) |
| 40 | m/z = | 49 | m/z = |
| | 657. 19 (C₄₅H₂₇N₃OS = 657. 79) | | 640.23 (C₄₅H₂₈N₄O = 640.75) |
| 50 | m/z = | 52 | m/z = |
| | 640.23 (C₄₅H₂₈N₄O = 640.75) | | 640.23 (C₄₅H₂₈N₄O = 640.75) |
| 53 | m/z = | 56 | m/z = |
| | 657. 19 (C₄₅H₂₇N₃OS = 657. 79) | | 657.19 (C₄₅H₂₇N₃OS = 657.79) |
| 57 | m/z = | 60 | m/z = |
| | 673.16 (C₄₅H₂₇N₃S₂ = 673.85) | | 673.16 (C₄₅H₂₇N₃S₂ = 673.85) |
| 64 | m/z = | 101 | m/z = |
| | 732.23 (C₅₁H₃₂N₄S = 732.90) | | 717.24 (C₅₁H₃₁N₃O₂ = 717.83) |
| 115 | m/z = | 132 | m/z = |
| | 717.24 (C₅₁H₃₁N₃O₂ = 717.83) | | 733.22 (C₅₁H₃₁N₃OS = 733.89) |
| 136 | m/z = | 152 | m/z = |
| | 717.24 (C₅₁H₃₁N₃O₂ = 717.83) | | 716.26 (C₅₁H₃₂N₄O = 716.84) |
| 153 | m/z = | 160 | m/z = |
| | 733.22 (C₅₁H₃₁N₃OS = 733.89) | | 749.20 (C₅₁H₃₁N₃S₂ = 749.95) |
| 165 | m/z = | 180 | m/z = |
| | 733.22 (C₅₁H₃₁N₃OS = 733.89) | | 733.22 (C₅₁H₃₁N₃OS = 733.89) |
| 203 | m/z = | 220 | m/z = |
| | 717.24 (C₅₁H₃₁N₃O₂ = 717.83) | | 733.22 (C₅₁H₃₁N₃OS = 733.89) |
| 226 | m/z = | 252 | m/z = |
| | 717.24 (C₅₁H₃₁N₃O₂ = 717.83) | | 716.26 (C₅₁H₃₂N₄O = 716.84) |
| 254 | m/z = | 301 | m/z = |
| | 733.22 (C₅₁H₃₁N₃OS = 733.89) | | 731.22 (C₅₁H₂₉N₃O₃ = 731.81) |
| 313 | m/z = | 327 | m/z = |
| | 731.22 (C₅₁H₂₉N₃O₃ = 731.81) | | 731.22 (C₅₁H₂₉N₃O₃ = 731.81) |
| 338 | m/z = | 352 | m/z = |
| | 747.20 (C₅₁H₂₉N₃O₂ S = 747.87) | | 730.24 (C₅₁H₃₀N₄O₂ = 730.83) |
| 354 | m/z = | 360 | m/z = |
| | 747.20 (C₅₁H₂₉N₃O₂ S = 747.87) | | 763.18 (C₅₁H₂₉N₃OS₂ = 763.93) |
| 368 | m/z = | 401 | m/z = |
| | 747.20 (C₅₁H₂₉N₃O₂ S = 747.87) | | 656.20 (C₄₅H₂₈N₄S = 656.81) |
| 409 | m/z = | 413 | m/z = |
| | 732.23 (C₅₁H₃₂N₄S = 732.90) | | 746.21 (C₅₁H₃₀N₄OS = 746.89) |
| 417 | m/z = | 438 | m/z = |
| | 792.29 (C₅₇H₃₆N₄O = 792.94) | | 793.27 (C₅₇H₃₅N₃O₂ = 793.93) |
| 468 | m/z = | 469 | m/z = |
| | 823.23 (C₅₇H₃N₃O₂S = 823. 97) | | 806.27 (C₅₇H₃₄N₄O₂ = 806.93) |
| 492 | m/z = | 511 | m/z = |
| | 716.26 (C₅₁H₃₂N₄O = 716.84) | | 717.24 (C₅₁H₃₁N₃O₂ = 717.83) |
| 534 | m/z = | 541 | m/z = |
| | 716.26 (C₅₁H₃₂N₄O = 716.84) | | 717.24 (C₅₁H₃₁N₃O₂ = 717.83) |
| 556 | m/z = | 569 | m/z = |
| | 717.24 (C₅₁H₃₁N₃O₂ = 717.83) | | 733.22 (C₅₁H₃₁N₃OS = 733.89) |
| 572 | m/z = | 596 | m/z = |
| | 733.22 (C₅₁H₃₁N₃OS = 733.89) | | 732.23 (C₅₁H₃₂N₄S = 732.90) |
| 2-50 | m/z = | 2-51 | m/z = |
| | 668.99 (C₄₈D₃₂N₂ = 668.46) | | 588.87 (C₄₂D₂₈N₂ = 588.40) |
| 2-53 | m/z = | 2-56 | m/z = |
| | 668.99 (C₄₈D₃₂N₂ = 668.46) | | 668.99 (C₄₈D₃₂N₂ = 668.46) |
| 2-57 | m/z = | 2-74 | m/z = |
| | 668.46 (C₄₈D₃₂N₂ = 668.99) | | 560.23 (C₄₂H₂₈N₂ = 560.70) |
| 2-76 | m/z = | 2-77 | m/z = |
| | 636.80 (C₄₈H₃₂N₂ = 636.26) | | 636.80 (C₄₈H₃₂N₂ = 636.26) |
| 2-78 | m/z = | 2 -79 | m/z = |
| | 636.80 (C₄₈H₃₂N₂ = 636.26) | | 636.80 (C₄₈H₃₂N₂ = 636.26) |

### Experimental Example 1

### 1) Manufacture of Organic Light Emitting Device

A glass substrate, in which ITO was thinly coated to have a thickness of 1,500 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. The light emitting layer was deposited to have a thickness of 360 Å by using a compound described in the following Table 7 as a host and tris(2-phenylpyridine)iridium (Ir(ppy)₃) as a green phosphorescent dopant to dope the host with Ir(ppy)₃ in an amount of 7%. Thereafter, BCP as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then an aluminum (Al) negative electrode was deposited to have a thickness of 1200 Å on the electron injection layer to form a negative electrode, thereby manufacturing an organic electroluminescence device.

In addition to those separately indicated as red hosts in the following Table 7, the Examples and Comparative Examples were used as green hosts. In the examples used as the red host, Ir(piq)₂(acac) was used as the red phosphorescent dopant.

Meanwhile, all the organic compounds required for manufacturing an OLED device were subjected to vacuum sublimed purification under 10⁻⁸ to 10⁻⁶ torr for each material, and used for the manufacture of OLED.

### Comparative Example Compound

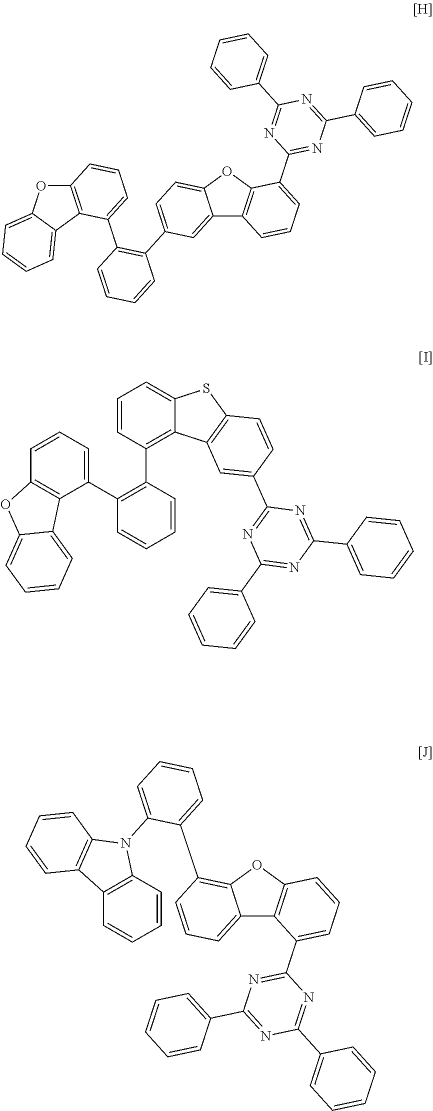 [SMILES: c1ccc(-c2nc(-c3ccccc3)nc(-c3cccc4c3oc3ccc(-c5ccccc5-c5cccc6oc7ccccc7c56)cc34)n2)cc1 ; c1ccc(-c2nc(-c3ccccc3)nc(-c3ccc4sc5cccc(-c6ccccc6-c6cccc7oc8ccccc8c67)c5c4c3)n2)cc1 ; c1ccc(-c2nc(-c3ccccc3)nc(-c3cccc4oc5c(-c6ccccc6-n6c7ccccc7c7ccccc76)cccc5c34)n2)cc1]

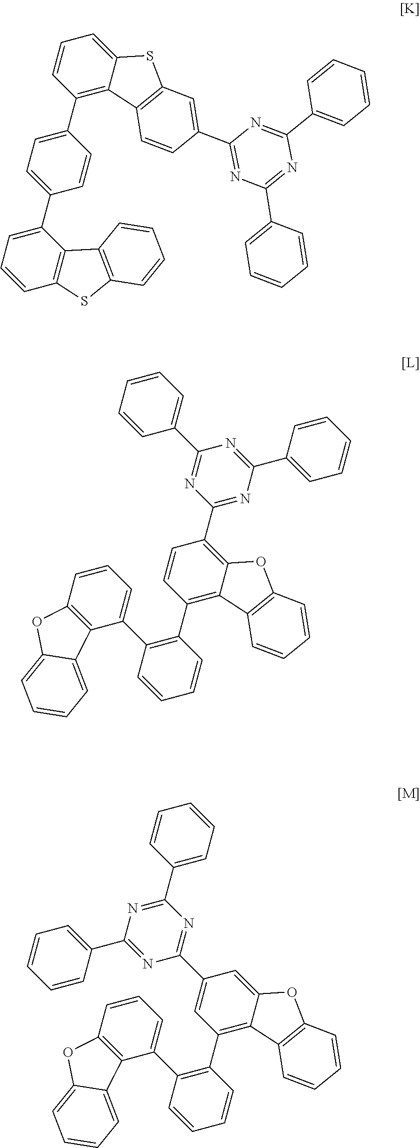 [SMILES: c1ccc(-c2nc(-c3ccccc3)nc(-c3ccc4c(c3)sc3cccc(-c5ccc(-c6cccc7sc8ccccc8c67)cc5)c34)n2)cc1 ; c1ccc(-c2nc(-c3ccccc3)nc(-c3ccc(-c4ccccc4-c4cccc5oc6ccccc6c45)c4c3oc3ccccc34)n2)cc1 ; c1ccc(-c2nc(-c3ccccc3)nc(-c3cc(-c4ccccc4-c4cccc5oc6ccccc6c45)c4c(c3)oc3ccccc34)n2)cc1]

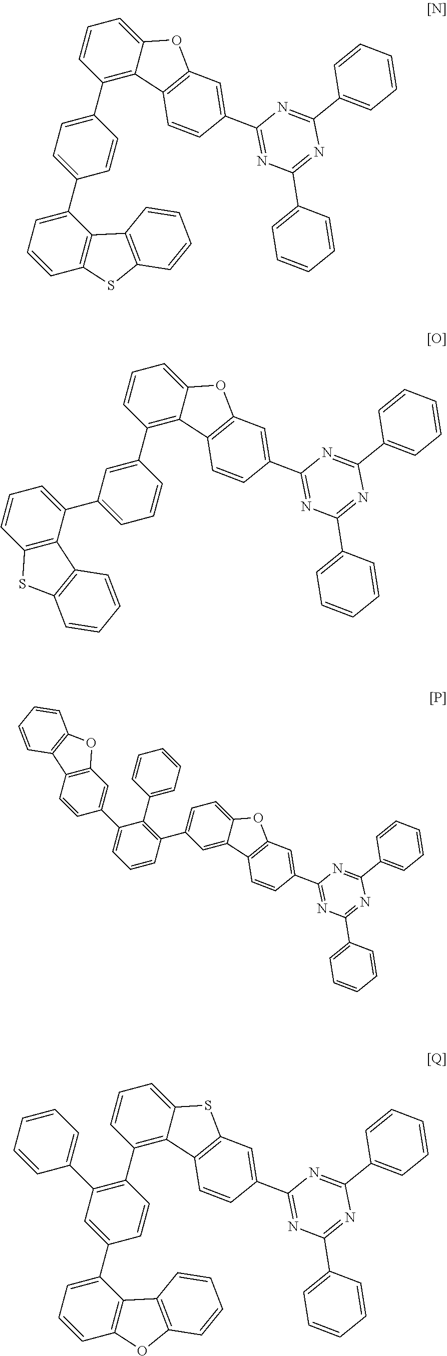 [SMILES: c1ccc(-c2nc(-c3ccccc3)nc(-c3ccc4c(c3)oc3cccc(-c5ccc(-c6cccc7sc8ccccc8c67)cc5)c34)n2)cc1 ; c1ccc(-c2nc(-c3ccccc3)nc(-c3ccc4c(c3)oc3cccc(-c5cccc(-c6cccc7sc8ccccc8c67)c5)c34)n2)cc1 ; c1ccc(-c2nc(-c3ccccc3)nc(-c3ccc4c(c3)oc3ccc(-c5cccc(-c6ccc7c(c6)oc6ccccc67)c5-c5ccccc5)cc34)n2)cc1 ; c1ccc(-c2nc(-c3ccccc3)nc(-c3ccc4c(c3)sc3cccc(-c5ccc(-c6cccc7oc8ccccc8c67)cc5-c5ccccc5)c34)n2)cc1]

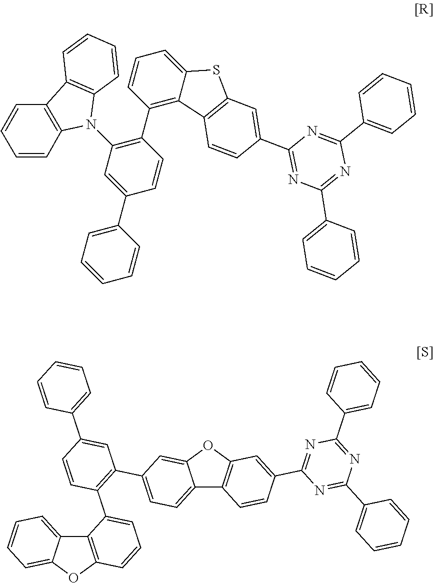 [SMILES: c1ccc(-c2ccc(-c3cccc4sc5cc(-c6nc(-c7ccccc7)nc(-c7ccccc7)n6)ccc5c34)c(-n3c4ccccc4c4ccccc43)c2)cc1 ; c1ccc(-c2ccc(-c3cccc4oc5ccccc5c34)c(-c3ccc4c(c3)oc3cc(-c5nc(-c6ccccc6)nc(-c6ccccc6)n5)ccc34)c2)cc1]

### 2) Driving Voltage and Light Emitting Efficiency of Organic Electroluminescence Device

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m².

The results of measuring the driving voltage, light emitting efficiency, color coordinate (CIE) and service life of the organic light emitting device manufactured according to the present invention are shown in the following Table 7.

TABLE 7

| | | | Light | | |
|---|---|---|---|---|---|
| | | Driving | emitting | | Service |
| | | voltage | efficiency | Color coordinate | life |
| No. | Compound | (V) | (cd/A) | (x, y) | (T90) |
| Example 1 | 1 | 4.58 | 70.4 | (0.242, 0.713) | 88 |
| Example 2 | 3 | 4.59 | 70.1 | (0.243, 0.714) | 85 |
| Example 3 | 5 | 4.60 | 70.9 | (0.244, 0.714) | 87 |
| Example 4 | 8 | 4.65 | 70.7 | (0.243, 0.693) | 86 |
| Example 5 | 10 | 4.58 | 71.4 | (0.251, 0.724) | 87 |
| Example 6 | 13 | 4.60 | 71.0 | (0.250, 0.724) | 87 |
| Example 7 | 16 | 4.48 | 72.2 | (0.242, 0.713) | 88 |
| Example 8 | 20 | 4.63 | 71.0 | (0.243, 0.712) | 86 |
| Example 9 | 24 | 4.54 | 71.5 | (0.242, 0.716) | 87 |
| Example 10 | 25 | 4.63 | 70.4 | (0.241, 0.713) | 86 |
| Example 11 | 32 | 4.51 | 71.3 | (0.248, 0.715) | 87 |
| Example 12 | 34 | 4.54 | 73.1 | (0.246, 0.717) | 89 |
| Example 13 | 40 | 4.62 | 72.5 | (0.231, 0.711) | 88 |
| Example 14 | 49 | 4.53 | 71.9 | (0.231, 0.711) | 87 |
| Example 15 | 50 | 4.58 | 71.3 | (0.251, 0.713) | 87 |
| Example 16 | 52 | 4.60 | 71.1 | (0.250, 0.713) | 88 |
| Example 17 | 53 | 4.64 | 71.0 | (0.245, 0.716) | 86 |
| Example 18 | 56 | 4.59 | 70.7 | (0.242, 0.713) | 86 |
| Example 19 | 57 | 4.60 | 71.0 | (0.242, 0.714) | 88 |
| Example 20 | 60 | 4.54 | 71.6 | (0.251, 0.724) | 87 |
| Example 21 | 64 | 4.53 | 72.2 | (0.243, 0.693) | 88 |
| Example 22 | 101 | 4.62 | 73.2 | (0.251, 0.714) | 89 |
| Example 23 | 115 | 4.64 | 73.0 | (0.247, 0.727) | 89 |
| Example 24 | 132 | 4.54 | 72.4 | (0.231, 0.711) | 88 |
| Example 25 | 136 | 4.50 | 71.6 | (0.246, 0.717) | 87 |
| Example 26 | 152 | 4.49 | 71.3 | (0.251, 0.724) | 87 |
| Example 27 | 153 | 4.54 | 71.9 | (0.243, 0.714) | 87 |
| Example 28 | 160 | 4.51 | 71.3 | (0.241, 0.711) | 87 |
| Example 29 | 165 | 4.60 | 73.2 | (0.241, 0.714) | 89 |
| Example 30 | 180 | 4.55 | 70.7 | (0.241, 0.715) | 86 |
| Example 31 | 203 | 4.51 | 71.3 | (0.231, 0.712) | 87 |
| Example 32 | 220 | 4.60 | 71.9 | (0.251, 0.714) | 87 |
| Example 33 | 226 | 4.48 | 72.5 | (0.241, 0.711) | 88 |
| Example 34 | 252 | 4.44 | 71.0 | (0.251, 0.714) | 86 |
| Example 35 | 254 | 4.59 | 71.6 | (0.242, 0.713) | 87 |
| Example 36 | 301 | 4.65 | 72.2 | (0.248, 0.715) | 88 |
| Example 37 | 313 | 4.60 | 72.8 | (0.251, 0.714) | 88 |
| Example 38 | 327 | 4.54 | 73.1 | (0.251, 0.714) | 89 |
| Example 39 | 338 | 4.51 | 70.4 | (0.247, 0.727) | 86 |
| Example 40 | 352 | 4.49 | 71.3 | (0.231, 0.711) | 87 |
| Example 41 | 354 | 4.60 | 72.5 | (0.246, 0.717) | 88 |
| Example 42 | 360 | 4.54 | 71.0 | (0.231, 0.711) | 86 |
| Example 43 | 368 | 4.51 | 72.2 | (0.671, 0.320) | 88 |
| Example 44 | 401 | 4.57 | 72.8 | (0.246, 0.717) | 88 |
| Example 45 | 409 | 4.57 | 71.5 | (0.233, 0.701) | 87 |
| Example 46 | 413 | 4.54 | 71.2 | (0.251, 0.713) | 86 |
| Example 47 | 417 | 4.60 | 72.1 | (0.254, 0.724) | 88 |
| Example 48 | 438 | 4.54 | 70.4 | (0.233, 0.703) | 86 |
| Example 49 | 468 | 4.65 | 71.6 | (0.234, 0.714) | 87 |
| Example 50 | 469 | 4.70 | 69.1 | (0.241, 0.714) | 81 |
| | (Red host) | | | | |
| Example 51 | 492 | 4.56 | 73.2 | (0.251, 0.724) | 89 |
| Example 52 | 511 | 4.54 | 73.0 | (0.242, 0.713) | 89 |
| Example 53 | 534 | 4.65 | 72.4 | (0.243, 0.712) | 88 |
| Example 54 | 541 | 4.59 | 71.6 | (0.242, 0.716) | 87 |
| Example 55 | 556 | 4.56 | 71.3 | (0.241, 0.713) | 87 |
| Example 56 | 569 | 4.63 | 71.0 | (0.248, 0.715) | 86 |
| Example 57 | 572 | 4.65 | 71.6 | (0.246, 0.717) | 87 |
| Example 58 | 596 | 4.53 | 72.2 | (0.231, 0.711) | 88 |
| Comparative | H | 5.05 | 63.5 | (0.251, 0.713) | 70 |
| Example 1 | | | | | |
| Comparative | I | 5.07 | 63.2 | (0.241, 0.711) | 71 |
| Example 2 | | | | | |
| Comparative | J | 5.10 | 63.1 | (0.251, 0.714) | 70 |
| Example 3 | | | | | |
| Comparative | K | 5.08 | 63.0 | (0.242, 0.713) | 72 |
| Example 4 | | | | | |
| Comparative | L | 5.06 | 62.9 | (0.248, 0.715) | 73 |
| Example 5 | | | | | |
| Comparative | M | 5.05 | 62.8 | (0.251, 0.714) | 71 |
| Example 6 | | | | | |
| Comparative | N | 5.11 | 63.1 | (0.251, 0.714) | 70 |
| Example 7 | | | | | |
| Comparative | O | 5.09 | 63.2 | (0.233, 0.701) | 71 |
| Example 8 | | | | | |
| Comparative | P | 5.07 | 63.5 | (0.251, 0.713) | 73 |
| Example 9 | | | | | |
| Comparative | Q | 5.05 | 63.1 | (0.254, 0.724) | 72 |
| Example 10 | | | | | |
| Comparative | R | 5.06 | 63.0 | (0.233, 0.703) | 72 |
| Example 11 | | | | | |
| Comparative | S | 5.09 | 62.9 | (0.234, 0.714) | 73 |
| Example 12 | | | | | |

Referring to the results of Table 7, it can be seen that an organic light emitting device including the heterocyclic compound of Chemical Formula 1 of the present invention has excellent driving voltage, light emitting efficiency and service life than the Comparative Examples. For the compounds used in Example 1 and Comparative Examples 5 and 6, Example 6 and Comparative Example 1, and Example 16 and Comparative Example 3, only the substitution position of the azine group is different and the other structures are the same, and it can be confirmed that the driving voltage of the Examples is lower, the light emitting efficiency is higher, and the service life is longer than each of the Comparative Examples. That is, as shown in Chemical Formula 1 of the present invention, it can be seen that by substituting an azine group at a specific position of a core structure, excellent performance is provided when Chemical Formula 1 of the present invention is used as a material for an organic light emitting device compared to compounds substituted at other positions.

Furthermore, for the compounds used in Example 3 and Comparative Examples 7 and 8, and Example 19 and Comparative Example 4, only the bond structure of the core structure-phenylene-substituent Het is different and the other structures are the same, and it can be confirmed that the examples in which the core structure-phenylene-substituent Het is bonded thereto at the ortho position have better results in terms of driving voltage, light emitting efficiency, and service life than the comparative examples in which the core structure-phenylene-substituent Het is bonded thereto at the meta or para position.

For the compound of Chemical Formula 1 of the present invention, the substituent Het is bonded to the core structure at the ortho position of the phenylene group, which increases the dihedral angle compared to when the substituent Het is at the meta or para position. As the dihedral angle increases, the extension of conjugation based on the phenylene group is suppressed, so that the compound of Chemical Formula 1 at the ortho position has a higher T₁ (triplet energy level) than the compound at the meta or para position. The high T₁ (triplet energy level) of a phosphorescent host material is suitable for maximizing the light emitting characteristics of a phosphorescent dopant. When a phosphorescent host material having a low T₁ (triplet energy level) is used, a back energy transfer occurs in which the electrons of the dopant in the triplet state are transferred back to the host, degrading the light emitting characteristics of the dopant to degrade the device performance. In the present invention, as shown in Chemical Formula 1, it is determined that by using a compound having a high T₁ (triplet energy level), in which the core structure and the substituent Het are bonded at the ortho position based on the phenylene group, the light emitting characteristics of the dopant are maximized by preventing back energy transfer, thereby improving the device performance.

In other words, it can be confirmed that when the heterocyclic compound of Chemical Formula 1 of the present invention is used as a host of a light emitting layer, the driving voltage, light emitting efficiency and service life are remarkably excellent.

### Experimental Example 2

### 1) Manufacture of Organic Light Emitting Device

A glass substrate, in which ITO was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. Pre-mixing was performed by pre-mixing two compounds (N-host and P-host) shown in the following Table 8 as hosts at the weight ratio shown in the following Table 8, and then the light emitting layer was deposited to have a thickness of 360 Å in one common container, and deposited by doping the host with Ir(ppy)₃ as a green phosphorescent dopant in an amount of 7% of the deposition thickness of the light emitting layer. Thereafter, BCP as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

In addition to those separately indicated as red hosts in the following Table 8, the Examples and Comparative Examples were used as green hosts. In the examples used as the red host, Ir(piq)₂(acac) was used as the red phosphorescent dopant.

Meanwhile, all the organic compounds required for manufacturing an OLED device were subjected to vacuum sublimed purification under 10⁻⁸ to 10⁻⁶ torr for each material, and used for the manufacture of OLED.

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/in².

The results of measuring the driving voltage, light emitting efficiency, color coordinate (CIE) and service life of the organic light emitting device manufactured according to the present invention are shown in the following Table 8.

TABLE 8

| | Light | | | Light | | |
|---|---|---|---|---|---|---|
| | emitting | | | emit- | | |
| | layer | | Driving | ting | | Ser- |
| | Com- | | volt- | effi- | Color | vice |
| | pound | Ratio | age | ciency | coordinate | life |
| No. | (N:P) | (N:P) | (V) | (cd/A) | (x, y) | (T90) |
| Example 59 | 8:2-76 | 1:1 | 4.25 | 79.2 | (0.248, 0.637) | 126 |
| Example 60 | | 1:2 | 4.30 | 79.0 | (0.269, 0.611) | 130 |
| Example 61 | | 1:3 | 4.35 | 78.8 | (0.251, 0.693) | 134 |
| Example 62 | 8:2-53 | 1:1 | 4.25 | 79.3 | (0.248, 0.637) | 187 |
| Example 63 | | 1:2 | 4.30 | 79.1 | (0.269, 0.611) | 195 |
| Example 64 | | 1:3 | 4.34 | 78.8 | (0.251, 0.693) | 202 |
| Example 65 | 34:2-74 | 1:1 | 4.15 | 81.9 | (0.269, 0.611) | 128 |
| Example 66 | | 1:2 | 4.20 | 81.5 | (0.251, 0.693) | 132 |
| Example 67 | | 1:3 | 4.25 | 81.1 | (0.248, 0.637) | 136 |
| Example 68 | 34:2-51 | 1:1 | 4.16 | 81.8 | (0.269, 0.611) | 190 |
| Example 69 | | 1:2 | 4.19 | 81.5 | (0.251, 0.693) | 197 |
| Example 70 | | 1:3 | 4.24 | 81.2 | (0.245, 0.677) | 204 |
| Example 71 | 49:2-77 | 1:1 | 4.14 | 80.5 | (0.258, 0.647) | 131 |
| Example 72 | | 1:2 | 4.18 | 80.3 | (0.266, 0.645) | 134 |
| Example 73 | | 1:3 | 4.22 | 79.9 | (0.245, 0.677) | 138 |
| Example 74 | 49:2-56 | 1:1 | 4.15 | 80.5 | (0.258, 0.647) | 194 |
| Example 75 | | 1:2 | 4.19 | 80.2 | (0.266, 0.645) | 201 |
| Example 76 | | 1:3 | 4.23 | 80.0 | (0.256, 0.673) | 208 |
| Example 77 | 64:2-79 | 1:1 | 4.14 | 80.9 | (0.237, 0.644) | 129 |
| Example 78 | | 1:2 | 4.16 | 80.5 | (0.237, 0.624) | 133 |
| Example 79 | | 1:3 | 4.18 | 80.2 | (0.256, 0.673) | 137 |
| Example 80 | 64:2-57 | 1:1 | 4.15 | 80.9 | (0.237, 0.644) | 192 |
| Example 81 | | 1:2 | 4.18 | 80.5 | (0.237, 0.624) | 199 |
| Example 82 | 64:2-57 | 1:3 | 4.20 | 80.2 | (0.245, 0.617) | 206 |
| Example 83 | 115:2-78 | 1:1 | 4.25 | 81.8 | (0.257, 0.624) | 128 |
| Example 84 | | 1:2 | 4.27 | 81.5 | (0.259, 0.712) | 132 |
| Example 85 | | 1:3 | 4.30 | 81.1 | (0.245, 0.617) | 136 |
| Example 86 | 115:2-50 | 1:1 | 4.25 | 81.8 | (0.257, 0.624) | 190 |
| Example 87 | | 1:2 | 4.28 | 81.6 | (0.259, 0.712) | 197 |
| Example 88 | | 1:3 | 4.30 | 81.2 | (0.243, 0.643) | 204 |
| Example 89 | 136:2-74 | 1:1 | 4.12 | 80.2 | (0.261, 0.764) | 129 |
| Example 90 | | 1:2 | 4.14 | 79.8 | (0.258, 0.628) | 133 |
| Example 91 | | 1:3 | 4.18 | 79.5 | (0.243, 0.643) | 137 |
| Example 92 | 136:2-51 | 1:1 | 4.11 | 80.1 | (0.261, 0.764) | 192 |
| Example 93 | | 1:2 | 4.14 | 79.9 | (0.258, 0.628) | 199 |
| Example 94 | | 1:3 | 4.19 | 79.6 | (0.254, 0.653) | 206 |
| Example 95 | 152:2-77 | 1:1 | 4.11 | 79.9 | (0.275, 0.657) | 131 |
| Example 96 | | 1:2 | 4.15 | 79.6 | (0.264, 0.642) | 135 |
| Example 97 | | 1:3 | 4.19 | 79.3 | (0.254, 0.653) | 139 |
| Example 98 | 152:2-56 | 1:1 | 4.10 | 79.9 | (0.275, 0.657) | 194 |
| Example 99 | | 1:2 | 4.14 | 79.6 | (0.264, 0.642) | 201 |
| Example 100 | | 1:3 | 4.18 | 79.4 | (0.256, 0.638) | 208 |
| Example 101 | 203:2-79 | 1:1 | 4.13 | 79.9 | (0.251, 0.632) | 132 |
| Example 102 | | 1:2 | 4.17 | 79.5 | (0.253, 0.684) | 136 |
| Example 103 | | 1:3 | 4.21 | 79.1 | (0.256, 0.638) | 140 |
| Example 104 | 203:2-57 | 1:1 | 4.13 | 79.9 | (0.251, 0.632) | 196 |
| Example 105 | | 1:2 | 4.18 | 79.6 | (0.253, 0.684) | 203 |
| Example 106 | | 1:3 | 4.21 | 79.2 | (0.235, 0.655) | 210 |
| Example 107 | 226:2-76 | 1:1 | 4.10 | 81.2 | (0.236, 0.624) | 126 |
| Example 108 | | 1:2 | 4.14 | 80.8 | (0.255, 0.692) | 130 |
| Example 109 | | 1:3 | 4.18 | 80.4 | (0.235, 0.655) | 134 |
| Example 110 | 226:2-53 | 1:1 | 4.10 | 81.2 | (0.236, 0.624) | 187 |
| Example 111 | | 1:2 | 4.13 | 80.9 | (0.255, 0.692) | 194 |
| Example 112 | | 1:3 | 4.17 | 80.4 | (0.253, 0.724) | 201 |
| Example 113 | 252:2-78 | 1:1 | 4.06 | 79.5 | (0.242, 0.625) | 128 |
| Example 114 | | 1:2 | 4.10 | 79.1 | (0.261, 0.623) | 132 |
| Example 115 | | 1:3 | 4.15 | 78.7 | (0.253, 0.724) | 136 |
| Example 116 | 252:2-50 | 1:1 | 4.06 | 79.5 | (0.242, 0.625) | 190 |
| Example 117 | | 1:2 | 4.09 | 79.2 | (0.261, 0.623) | 197 |
| Example 118 | | 1:3 | 4.14 | 78.8 | (0.253, 0.614) | 204 |
| Example 119 | 313:2-74 | 1:1 | 4.21 | 81.5 | (0.254, 0.659) | 129 |
| Example 120 | | 1:2 | 4.24 | 81.1 | (0.255, 0.635) | 133 |
| Example 121 | | 1:3 | 4.29 | 80.7 | (0.253, 0.614) | 137 |
| Example 122 | 313:2-51 | 1:1 | 4.20 | 81.5 | (0.254, 0.659) | 192 |
| Example 123 | | 1:2 | 4.23 | 81.2 | (0.255, 0.635) | 199 |
| Example 124 | | 1:3 | 4.28 | 80.8 | (0.257, 0.714) | 206 |
| Example 125 | 338:2-77 | 1:1 | 4.13 | 78.8 | (0.249, 0.666) | 131 |
| Example 126 | | 1:2 | 4.17 | 78.4 | (0.253, 0.635) | 135 |
| Example 127 | | 1:3 | 4.21 | 78.0 | (0.257, 0.714) | 139 |
| Example 128 | 338:2-56 | 1:1 | 4.12 | 78.8 | (0.249, 0.666) | 194 |
| Example 129 | 338:2-56 | 1:2 | 4.16 | 78.4 | (0.253, 0.635) | 201 |
| Example 130 | 338:2-56 | 1:3 | 4.20 | 78.1 | (0.268, 0.615) | 208 |
| Example 131 | 352:2-79 | 1:1 | 4.11 | 79.9 | (0.253, 0.628) | 132 |
| Example 132 | | 1:2 | 4.15 | 79.5 | (0.256, 0.713) | 136 |
| Example 133 | | 1:3 | 4.19 | 79.1 | (0.268, 0.615) | 140 |
| Example 134 | 352:2-57 | 1:1 | 4.12 | 79.9 | (0.253, 0.628) | 196 |
| Example 135 | | 1:2 | 4.16 | 79.5 | (0.256, 0.713) | 203 |
| Example 136 | | 1:3 | 4.20 | 79.1 | (0.243, 0.612) | 210 |
| Example 137 | 438:2-78 | 1:1 | 4.15 | 78.8 | (0.265, 0.669) | 131 |
| Example 138 | | 1:2 | 4.19 | 78.4 | (0.255, 0.627) | 135 |
| Example 139 | | 1:3 | 4.23 | 78.0 | (0.243, 0.612) | 139 |
| Example 140 | 438:2-50 | 1:1 | 4.15 | 78.8 | (0.265, 0.669) | 194 |
| Example 141 | | 1:2 | 4.18 | 78.5 | (0.255, 0.627) | 201 |
| Example 142 | | 1:3 | 4.22 | 78.1 | (0.243, 0.653) | 208 |
| Example 143 | 469:2-76 | 1:1 | 4.30 | 77.4 | (0.247, 0.644) | 120 |
| Example 144 | (Red | 1:2 | 4.33 | 77.0 | (0.274, 0.658) | 124 |
| Example 145 | host) | 1:3 | 4.37 | 76.7 | (0.243, 0.653) | 128 |
| Comparative | H:2-77 | 1:1 | 4.80 | 67.3 | (0.236, 0.624) | 86 |
| Example 13 | | | | | | |
| Comparative | | 1:2 | 4.84 | 66.9 | (0.255, 0.692) | 90 |
| Example 14 | | | | | | |
| Comparative | | 1:3 | 4.88 | 66.5 | (0.253, 0.724) | 94 |
| Example 15 | | | | | | |
| Comparative | K:2-79 | 1:1 | 4.75 | 66.8 | (0.242, 0.625) | 88 |
| Example 16 | | | | | | |
| Comparative | | 1:2 | 4.79 | 66.4 | (0.261, 0.623) | 92 |
| Example 17 | | | | | | |
| Comparative | | 1:3 | 4.83 | 66.0 | (0.253, 0.724) | 96 |
| Example 18 | | | | | | |
| Comparative | N:2-78 | 1:1 | 4.85 | 66.9 | (0.242, 0.625) | 86 |
| Example 19 | | | | | | |
| Comparative | | 1:2 | 4.88 | 66.5 | (0.261, 0.623) | 90 |
| Example 20 | | | | | | |
| Comparative | | 1:3 | 4.91 | 66.4 | (0.253, 0.614) | 98 |
| Example 21 | | | | | | |
| Comparative | | 1:1 | 4.81 | 66.8 | (0.254, 0.659) | 88 |
| Example 22 | | | | | | |
| Comparative | R:2-76 | 1:2 | 4.85 | 66.4 | (0.255, 0.635) | 92 |
| Example 23 | | | | | | |
| Comparative | | 1:3 | 4.89 | 66.0 | (0.253, 0.614) | 96 |
| Example 24 | | | | | | |

Comparing the results of Table 7 with those of Table 8, it can be confirmed that when the heterocyclic compound of Chemical Formula 1 and the compound of Chemical Formula 2 are used simultaneously as hosts in the light emitting layer, specifically, when the heterocyclic compound of Chemical Formula 1 is used as an N-type host and the compound of Chemical Formula 2 is used as a P-type host, all of the driving voltage, light emitting efficiency and service life are improved.

From these results, it can be expected that an exciplex phenomenon will occur when both compounds are included.

The exciplex phenomenon is a phenomenon in which energy with a magnitude of the HOMO level of a donor (p-host) and the LUMO level of an acceptor (n-host) is released due to an electron exchange between two molecules. When the exciplex phenomenon between two molecules occurs, a reverse intersystem crossing (RISC) occurs, and the internal quantum efficiency of fluorescence can be increased to 100% due to the RISC. When a donor with a good hole transport capacity (p-host) and an acceptor with a good electron transport capacity (n-host) are used as hosts for the light emitting layer, holes are injected into the p-host and electrons are injected into the n-host, so that the driving voltage can be lowered, which can help to improve the service life. In the present invention, it could be confirmed that the heterocyclic compound of Chemical Formula 1 serves as an acceptor and the compound of Chemical Formula 2 serves as a donor, so that when the compounds were used as a host in a light emitting layer, excellent device characteristics were exhibited.

In particular, it can be confirmed that when the compound of Chemical Formula 2 includes deuterium, service life characteristics are excellent. In Examples 59 to 142 in Table 8, for 14 compounds of Chemical Formula 1, two compounds different in the inclusion of deuterium in the same structure were used in combination as P-type hosts, and from the results in Table 8, it can be seen that when the heterocyclic compound of Chemical Formula 1 is used in combination with the compound of Chemical Formula 2 including deuterium, the service life is remarkably improved. This is determined that when the compound includes deuterium relative to the same structure, the compound shows much more balanced charge transport characteristics than a compound that does not include deuterium, and the stability of the entire molecule is increased due to the high single bond dissociation energy of carbon and deuterium, thereby increasing the service life.

In contrast, it can be seen that when the compounds out of the scope of the present invention are used in combination with the compound of Chemical Formula 2 (Comparative Examples 13 to 24), the performance in terms of driving voltage, light emitting efficiency and service life deteriorates compared to the present invention.

That is, it can be confirmed that when both the heterocyclic compound of Chemical Formula 1 and the compound of Chemical Formula 2 of the present invention are used as hosts of a light emitting layer, the driving voltage, light emitting efficiency and service life are remarkably excellent.

1. A heterocyclic compound of the following Chemical Formula 1: 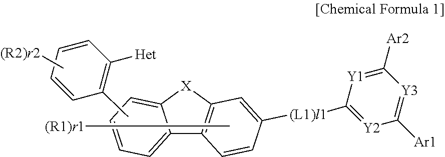
wherein, in Chemical Formula 1,
X is O or S,
L1 is a direct bond; a substituted or unsubstituted C6 to C60 arylene group; or a substituted or unsubstituted C2 to C60 heteroarylene group,
Y1 to Y3 are each independently N or CR, and at least one thereof is N,
Het is a substituted or unsubstituted C2 to C60 fused polycyclic heteroaryl group,
R, R1, Ar1, and Ar2 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R2 is hydrogen; or deuterium,
l1 is an integer from 0 to 3,
r1 is an integer from 0 to 6,
r2 is an integer from 0 to 4, and
when l1, r1, and r2 are each 2 or higher, substituents in the parenthesis are the same as or different from each other.

2. The heterocyclic compound of claim 1, wherein Chemical Formula 1 is represented by any one of the following Chemical Formulae 1-1 to 1-4: 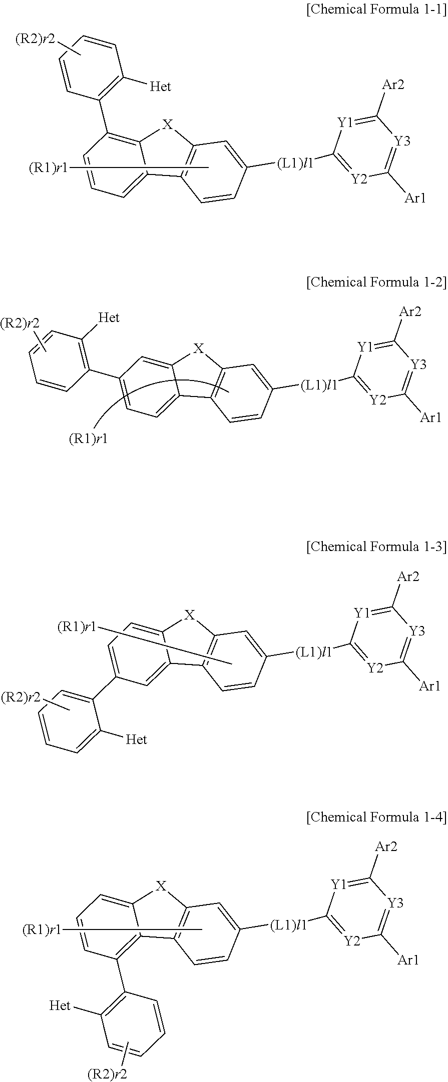
in Chemical Formulae 1-1 to 1-4, the definition of each substituent is the same as that in Chemical Formula 1.

3. The heterocyclic compound of claim 1, wherein Het is a substituted or unsubstituted C2 to C30 fused polycyclic heteroaryl group.

4. The heterocyclic compound of claim 1, wherein Ar1 and Ar2 are each independently a substituted or unsubstituted C6 to C30 aryl group; or a substituted or unsubstituted C2 to C30 heteroaryl group.

5. The heterocyclic compound of claim 1, wherein a deuterium content of Chemical Formula 1 is 0%, or 20% to 100%.

6. The heterocyclic compound of claim 1, wherein Chemical Formula 1 is represented by any one of the following compounds:

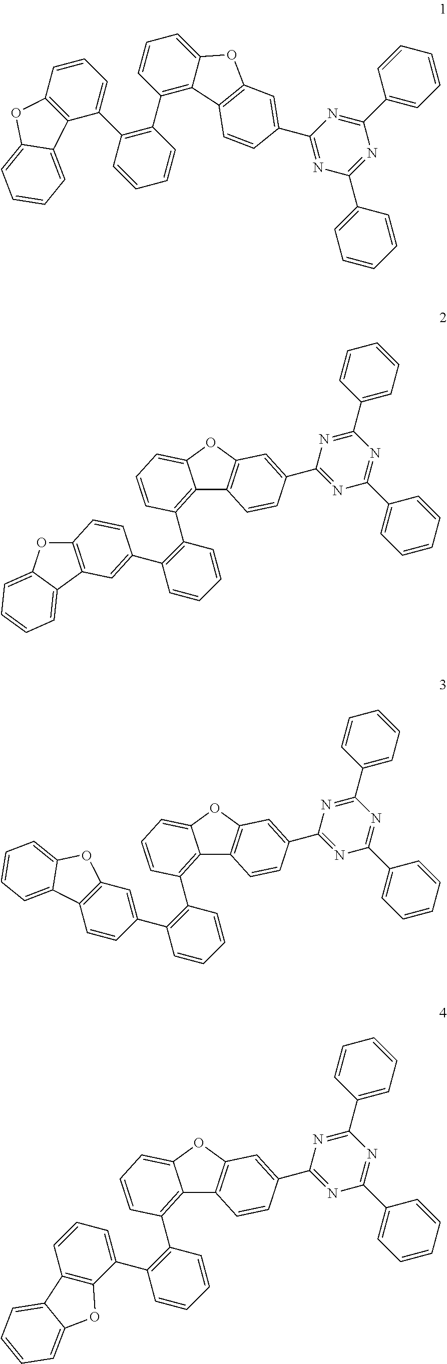 [SMILES: c1ccc(-c2nc(-c3ccccc3)nc(-c3ccc4c(c3)oc3cccc(-c5ccccc5-c5cccc6oc7ccccc7c56)c34)n2)cc1 ; c1ccc(-c2nc(-c3ccccc3)nc(-c3ccc4c(c3)oc3cccc(-c5ccccc5-c5ccc6oc7ccccc7c6c5)c34)n2)cc1 ; c1ccc(-c2nc(-c3ccccc3)nc(-c3ccc4c(c3)oc3cccc(-c5ccccc5-c5ccc6c(c5)oc5ccccc56)c34)n2)cc1 ; c1ccc(-c2nc(-c3ccccc3)nc(-c3ccc4c(c3)oc3cccc(-c5ccccc5-c5cccc6c5oc5ccccc56)c34)n2)cc1]

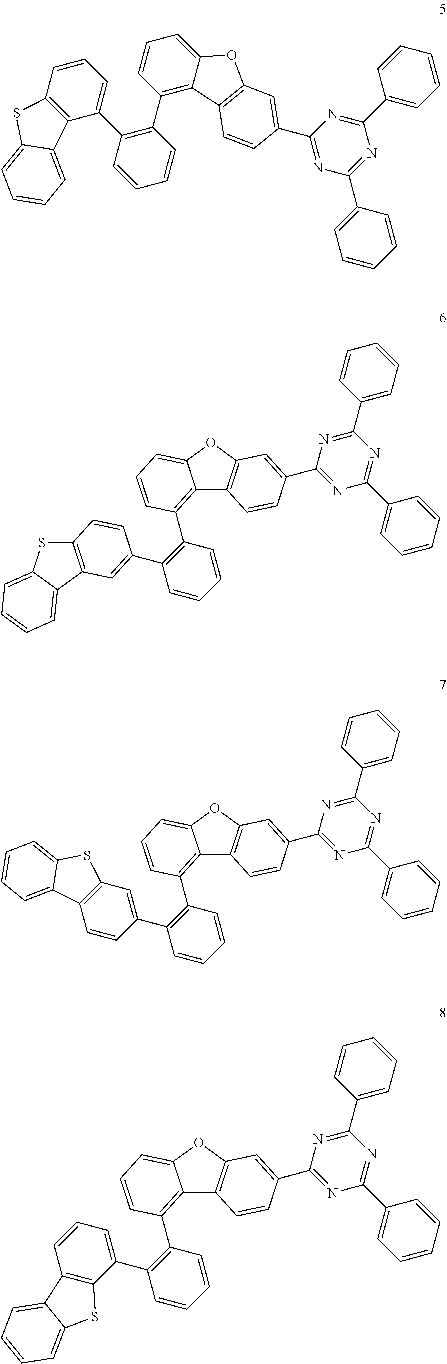 [SMILES: c1ccc(-c2nc(-c3ccccc3)nc(-c3ccc4c(c3)oc3cccc(-c5ccccc5-c5cccc6sc7ccccc7c56)c34)n2)cc1 ; c1ccc(-c2nc(-c3ccccc3)nc(-c3ccc4c(c3)oc3cccc(-c5ccccc5-c5ccc6sc7ccccc7c6c5)c34)n2)cc1 ; c1ccc(-c2nc(-c3ccccc3)nc(-c3ccc4c(c3)oc3cccc(-c5ccccc5-c5ccc6c(c5)sc5ccccc56)c34)n2)cc1 ; c1ccc(-c2nc(-c3ccccc3)nc(-c3ccc4c(c3)oc3cccc(-c5ccccc5-c5cccc6c5sc5ccccc56)c34)n2)cc1]

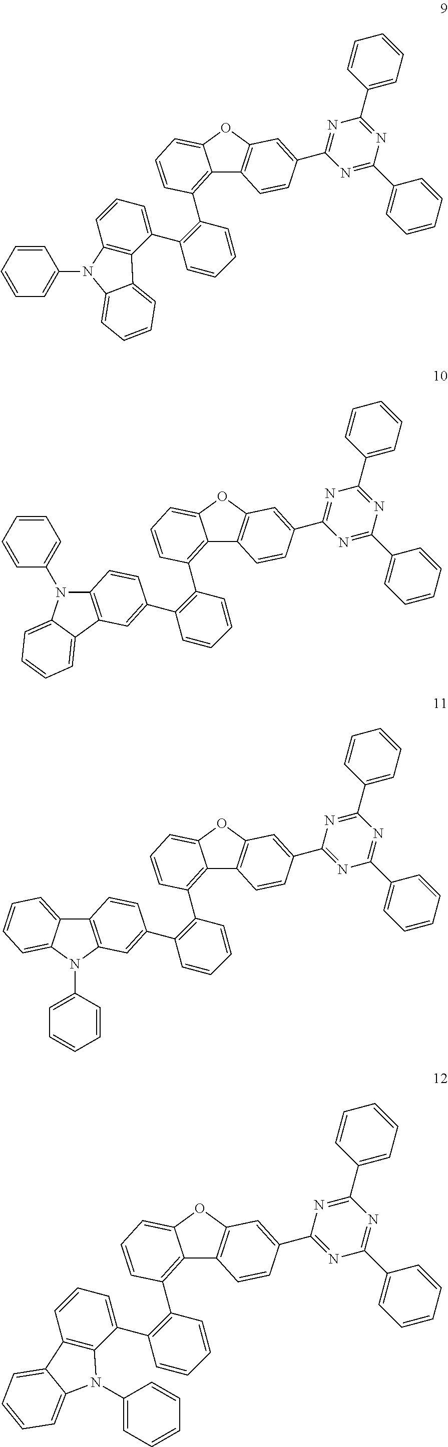 [SMILES: c1ccc(-c2nc(-c3ccccc3)nc(-c3ccc4c(c3)oc3cccc(-c5ccccc5-c5cccc6c5c5ccccc5n6-c5ccccc5)c34)n2)cc1 ; c1ccc(-c2nc(-c3ccccc3)nc(-c3ccc4c(c3)oc3cccc(-c5ccccc5-c5ccc6c(c5)c5ccccc5n6-c5ccccc5)c34)n2)cc1 ; c1ccc(-c2nc(-c3ccccc3)nc(-c3ccc4c(c3)oc3cccc(-c5ccccc5-c5ccc6c7ccccc7n(-c7ccccc7)c6c5)c34)n2)cc1 ; c1ccc(-c2nc(-c3ccccc3)nc(-c3ccc4c(c3)oc3cccc(-c5ccccc5-c5cccc6c7ccccc7n(-c7ccccc7)c56)c34)n2)cc1]

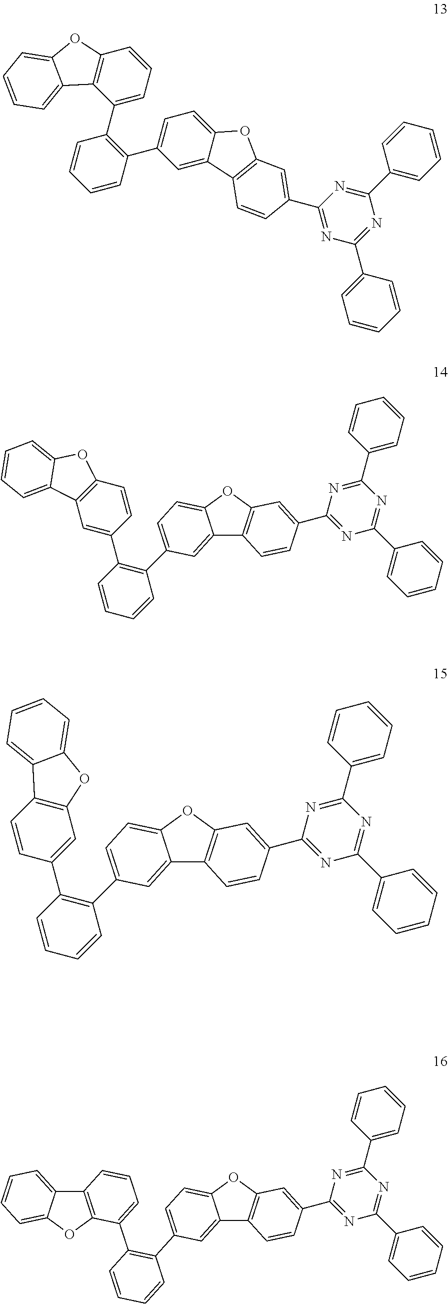 [SMILES: c1ccc(-c2nc(-c3ccccc3)nc(-c3ccc4c(c3)oc3ccc(-c5ccccc5-c5cccc6oc7ccccc7c56)cc34)n2)cc1 ; c1ccc(-c2nc(-c3ccccc3)nc(-c3ccc4c(c3)oc3ccc(-c5ccccc5-c5ccc6oc7ccccc7c6c5)cc34)n2)cc1 ; c1ccc(-c2nc(-c3ccccc3)nc(-c3ccc4c(c3)oc3ccc(-c5ccccc5-c5ccc6c(c5)oc5ccccc56)cc34)n2)cc1 ; c1ccc(-c2nc(-c3ccccc3)nc(-c3ccc4c(c3)oc3ccc(-c5ccccc5-c5cccc6c5oc5ccccc56)cc34)n2)cc1]

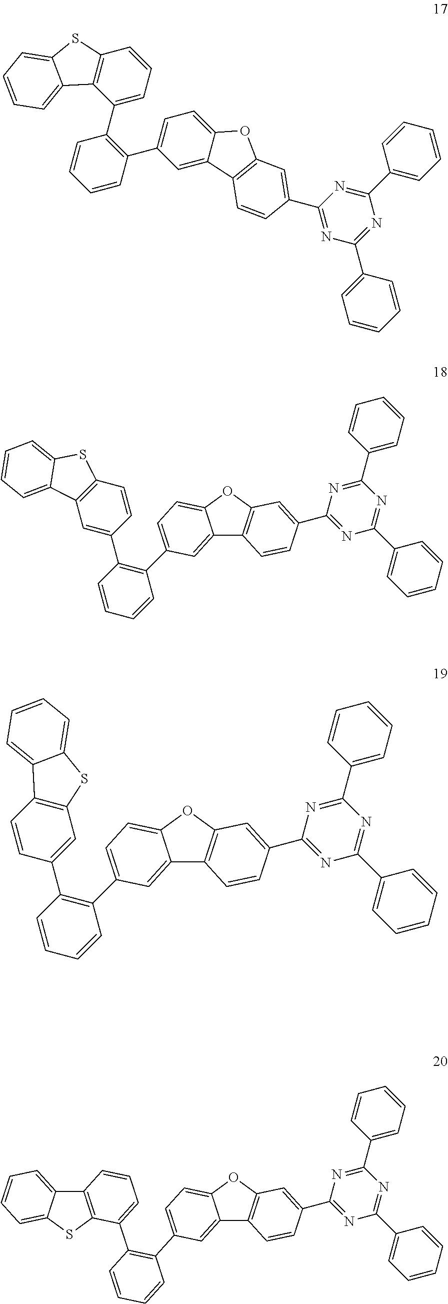 [SMILES: c1ccc(-c2nc(-c3ccccc3)nc(-c3ccc4c(c3)oc3ccc(-c5ccccc5-c5cccc6sc7ccccc7c56)cc34)n2)cc1 ; c1ccc(-c2nc(-c3ccccc3)nc(-c3ccc4c(c3)oc3ccc(-c5ccccc5-c5ccc6sc7ccccc7c6c5)cc34)n2)cc1 ; c1ccc(-c2nc(-c3ccccc3)nc(-c3ccc4c(c3)oc3ccc(-c5ccccc5-c5ccc6c(c5)sc5ccccc56)cc34)n2)cc1 ; c1ccc(-c2nc(-c3ccccc3)nc(-c3ccc4c(c3)oc3ccc(-c5ccccc5-c5cccc6c5sc5ccccc56)cc34)n2)cc1]

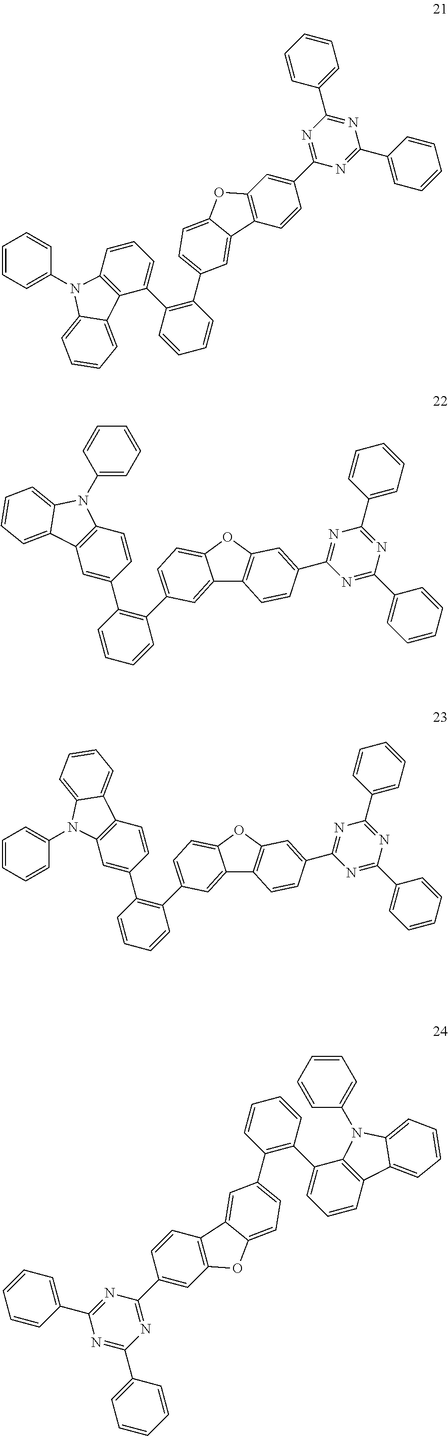 [SMILES: c1ccc(-c2nc(-c3ccccc3)nc(-c3ccc4c(c3)oc3ccc(-c5ccccc5-c5cccc6c5c5ccccc5n6-c5ccccc5)cc34)n2)cc1 ; c1ccc(-c2nc(-c3ccccc3)nc(-c3ccc4c(c3)oc3ccc(-c5ccccc5-c5ccc6c(c5)c5ccccc5n6-c5ccccc5)cc34)n2)cc1 ; c1ccc(-c2nc(-c3ccccc3)nc(-c3ccc4c(c3)oc3ccc(-c5ccccc5-c5ccc6c7ccccc7n(-c7ccccc7)c6c5)cc34)n2)cc1 ; c1ccc(-c2nc(-c3ccccc3)nc(-c3ccc4c(c3)oc3ccc(-c5ccccc5-c5cccc6c7ccccc7n(-c7ccccc7)c56)cc34)n2)cc1]

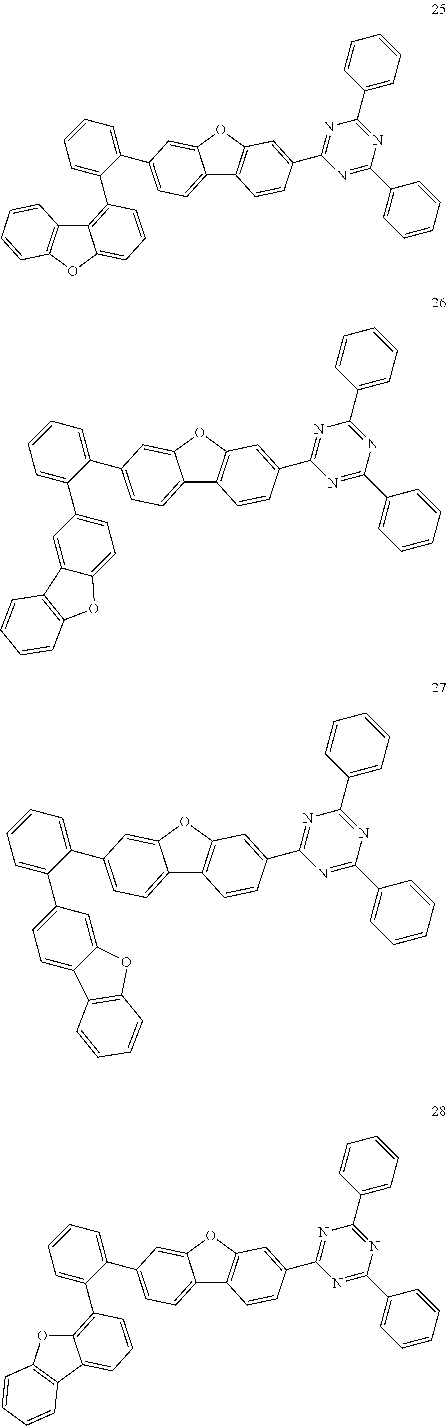 [SMILES: c1ccc(-c2nc(-c3ccccc3)nc(-c3ccc4c(c3)oc3cc(-c5ccccc5-c5cccc6oc7ccccc7c56)ccc34)n2)cc1 ; c1ccc(-c2nc(-c3ccccc3)nc(-c3ccc4c(c3)oc3cc(-c5ccccc5-c5ccc6oc7ccccc7c6c5)ccc34)n2)cc1 ; c1ccc(-c2nc(-c3ccccc3)nc(-c3ccc4c(c3)oc3cc(-c5ccccc5-c5ccc6c(c5)oc5ccccc56)ccc34)n2)cc1 ; c1ccc(-c2nc(-c3ccccc3)nc(-c3ccc4c(c3)oc3cc(-c5ccccc5-c5cccc6c5oc5ccccc56)ccc34)n2)cc1]

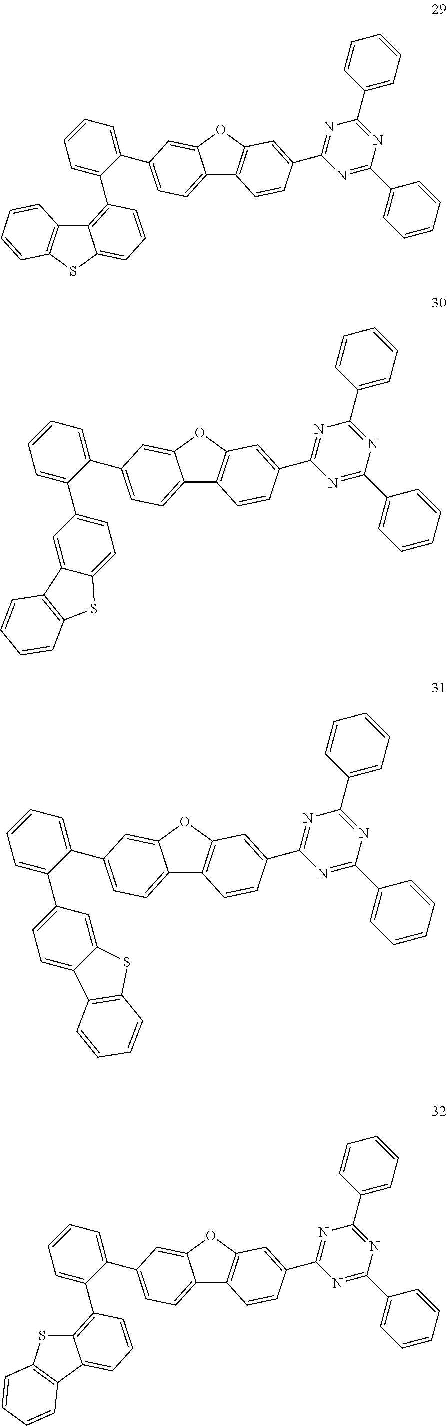 [SMILES: c1ccc(-c2nc(-c3ccccc3)nc(-c3ccc4c(c3)oc3cc(-c5ccccc5-c5cccc6sc7ccccc7c56)ccc34)n2)cc1 ; c1ccc(-c2nc(-c3ccccc3)nc(-c3ccc4c(c3)oc3cc(-c5ccccc5-c5ccc6sc7ccccc7c6c5)ccc34)n2)cc1 ; c1ccc(-c2nc(-c3ccccc3)nc(-c3ccc4c(c3)oc3cc(-c5ccccc5-c5ccc6c(c5)sc5ccccc56)ccc34)n2)cc1 ; c1ccc(-c2nc(-c3ccccc3)nc(-c3ccc4c(c3)oc3cc(-c5ccccc5-c5cccc6c5sc5ccccc56)ccc34)n2)cc1]

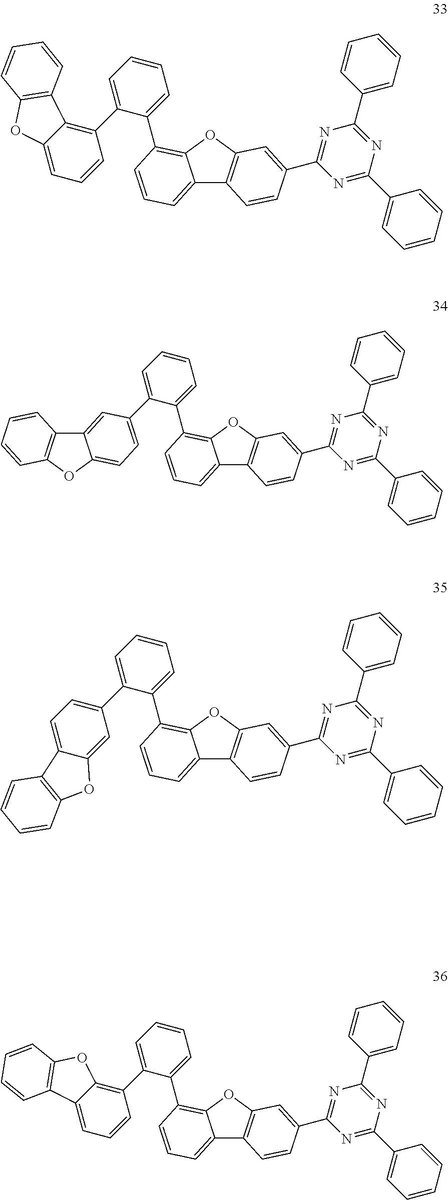 [SMILES: c1ccc(-c2nc(-c3ccccc3)nc(-c3ccc4c(c3)oc3c(-c5ccccc5-c5cccc6oc7ccccc7c56)cccc34)n2)cc1 ; c1ccc(-c2nc(-c3ccccc3)nc(-c3ccc4c(c3)oc3c(-c5ccccc5-c5ccc6oc7ccccc7c6c5)cccc34)n2)cc1 ; c1ccc(-c2nc(-c3ccccc3)nc(-c3ccc4c(c3)oc3c(-c5ccccc5-c5ccc6c(c5)oc5ccccc56)cccc34)n2)cc1 ; c1ccc(-c2nc(-c3ccccc3)nc(-c3ccc4c(c3)oc3c(-c5ccccc5-c5cccc6c5oc5ccccc56)cccc34)n2)cc1]

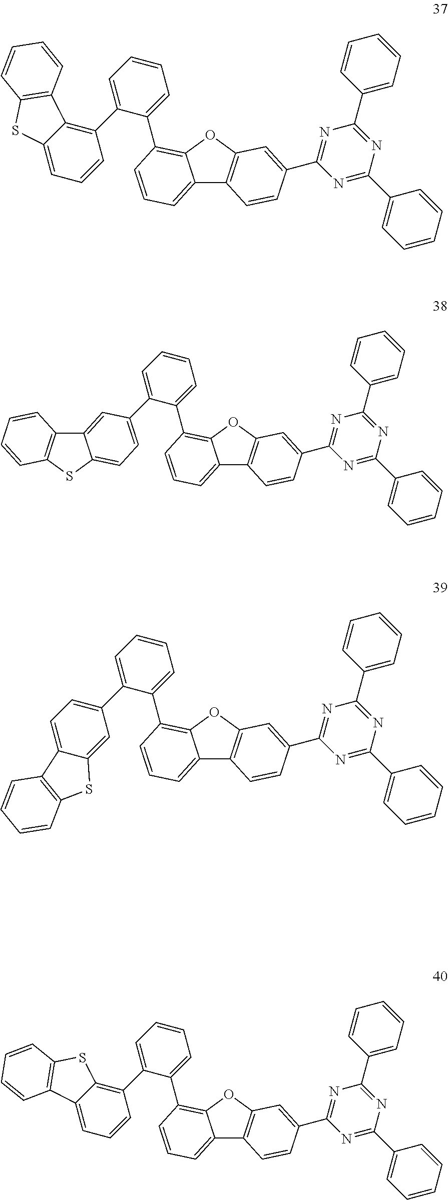 [SMILES: c1ccc(-c2nc(-c3ccccc3)nc(-c3ccc4c(c3)oc3c(-c5ccccc5-c5cccc6sc7ccccc7c56)cccc34)n2)cc1 ; c1ccc(-c2nc(-c3ccccc3)nc(-c3ccc4c(c3)oc3c(-c5ccccc5-c5ccc6sc7ccccc7c6c5)cccc34)n2)cc1 ; c1ccc(-c2nc(-c3ccccc3)nc(-c3ccc4c(c3)oc3c(-c5ccccc5-c5ccc6c(c5)sc5ccccc56)cccc34)n2)cc1 ; c1ccc(-c2nc(-c3ccccc3)nc(-c3ccc4c(c3)oc3c(-c5ccccc5-c5cccc6c5sc5ccccc56)cccc34)n2)cc1]

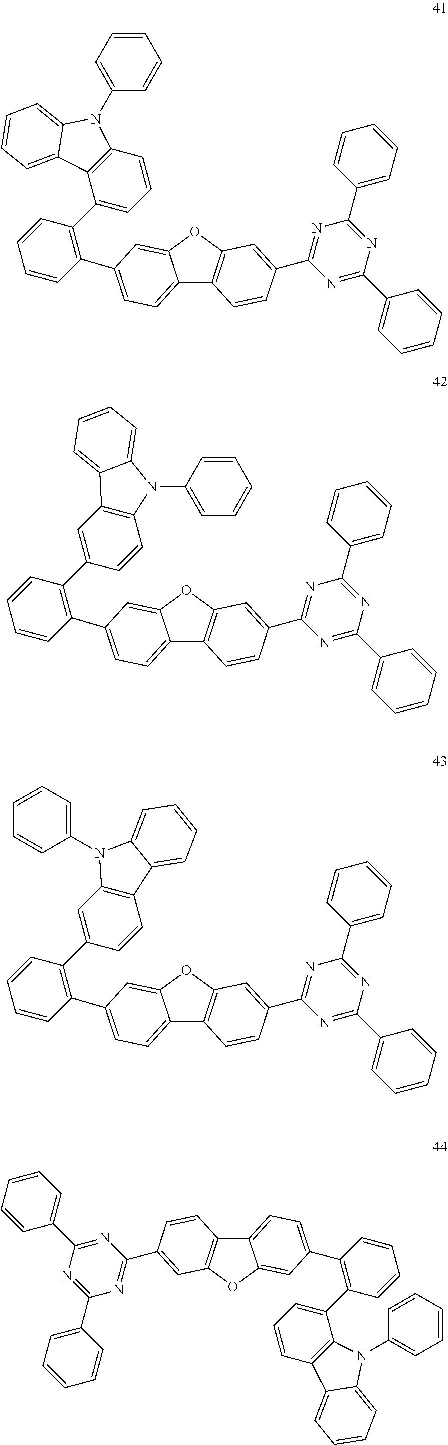 [SMILES: c1ccc(-c2nc(-c3ccccc3)nc(-c3ccc4c(c3)oc3cc(-c5ccccc5-c5cccc6c5c5ccccc5n6-c5ccccc5)ccc34)n2)cc1 ; c1ccc(-c2nc(-c3ccccc3)nc(-c3ccc4c(c3)oc3cc(-c5ccccc5-c5ccc6c(c5)c5ccccc5n6-c5ccccc5)ccc34)n2)cc1 ; c1ccc(-c2nc(-c3ccccc3)nc(-c3ccc4c(c3)oc3cc(-c5ccccc5-c5ccc6c7ccccc7n(-c7ccccc7)c6c5)ccc34)n2)cc1 ; c1ccc(-c2nc(-c3ccccc3)nc(-c3ccc4c(c3)oc3cc(-c5ccccc5-c5cccc6c7ccccc7n(-c7ccccc7)c56)ccc34)n2)cc1]

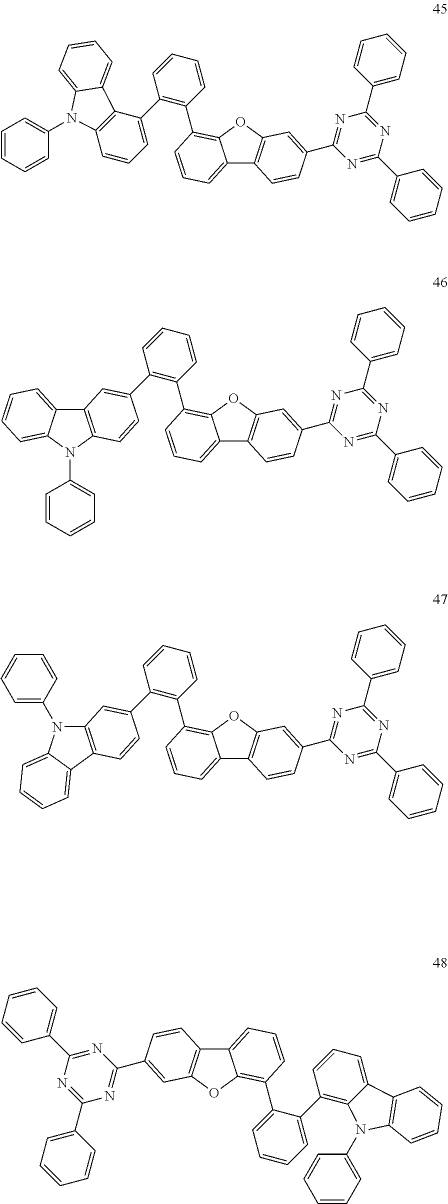 [SMILES: c1ccc(-c2nc(-c3ccccc3)nc(-c3ccc4c(c3)oc3c(-c5ccccc5-c5cccc6c5c5ccccc5n6-c5ccccc5)cccc34)n2)cc1 ; c1ccc(-c2nc(-c3ccccc3)nc(-c3ccc4c(c3)oc3c(-c5ccccc5-c5ccc6c(c5)c5ccccc5n6-c5ccccc5)cccc34)n2)cc1 ; c1ccc(-c2nc(-c3ccccc3)nc(-c3ccc4c(c3)oc3c(-c5ccccc5-c5ccc6c7ccccc7n(-c7ccccc7)c6c5)cccc34)n2)cc1 ; c1ccc(-c2nc(-c3ccccc3)nc(-c3ccc4c(c3)oc3c(-c5ccccc5-c5cccc6c7ccccc7n(-c7ccccc7)c56)cccc34)n2)cc1]

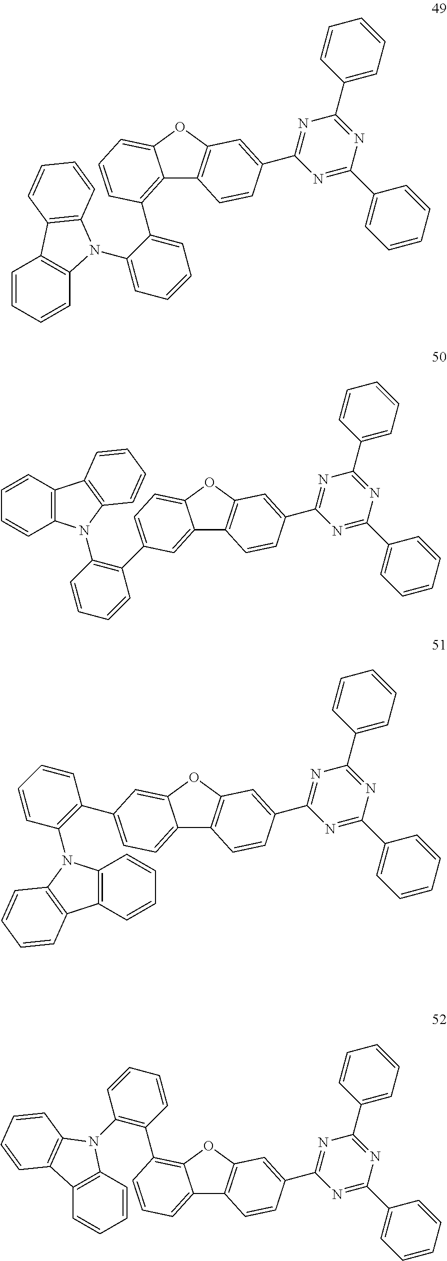 [SMILES: c1ccc(-c2nc(-c3ccccc3)nc(-c3ccc4c(c3)oc3cccc(-c5ccccc5-n5c6ccccc6c6ccccc65)c34)n2)cc1 ; c1ccc(-c2nc(-c3ccccc3)nc(-c3ccc4c(c3)oc3ccc(-c5ccccc5-n5c6ccccc6c6ccccc65)cc34)n2)cc1 ; c1ccc(-c2nc(-c3ccccc3)nc(-c3ccc4c(c3)oc3cc(-c5ccccc5-n5c6ccccc6c6ccccc65)ccc34)n2)cc1 ; c1ccc(-c2nc(-c3ccccc3)nc(-c3ccc4c(c3)oc3c(-c5ccccc5-n5c6ccccc6c6ccccc65)cccc34)n2)cc1]

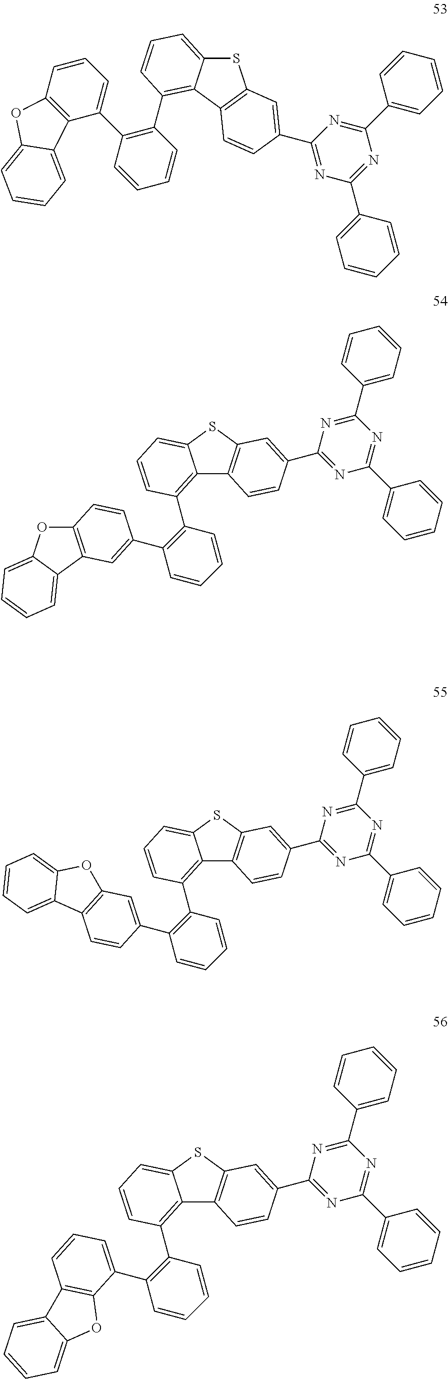 [SMILES: c1ccc(-c2nc(-c3ccccc3)nc(-c3ccc4c(c3)sc3cccc(-c5ccccc5-c5cccc6oc7ccccc7c56)c34)n2)cc1 ; c1ccc(-c2nc(-c3ccccc3)nc(-c3ccc4c(c3)sc3cccc(-c5ccccc5-c5ccc6oc7ccccc7c6c5)c34)n2)cc1 ; c1ccc(-c2nc(-c3ccccc3)nc(-c3ccc4c(c3)sc3cccc(-c5ccccc5-c5ccc6c(c5)oc5ccccc56)c34)n2)cc1 ; c1ccc(-c2nc(-c3ccccc3)nc(-c3ccc4c(c3)sc3cccc(-c5ccccc5-c5cccc6c5oc5ccccc56)c34)n2)cc1]

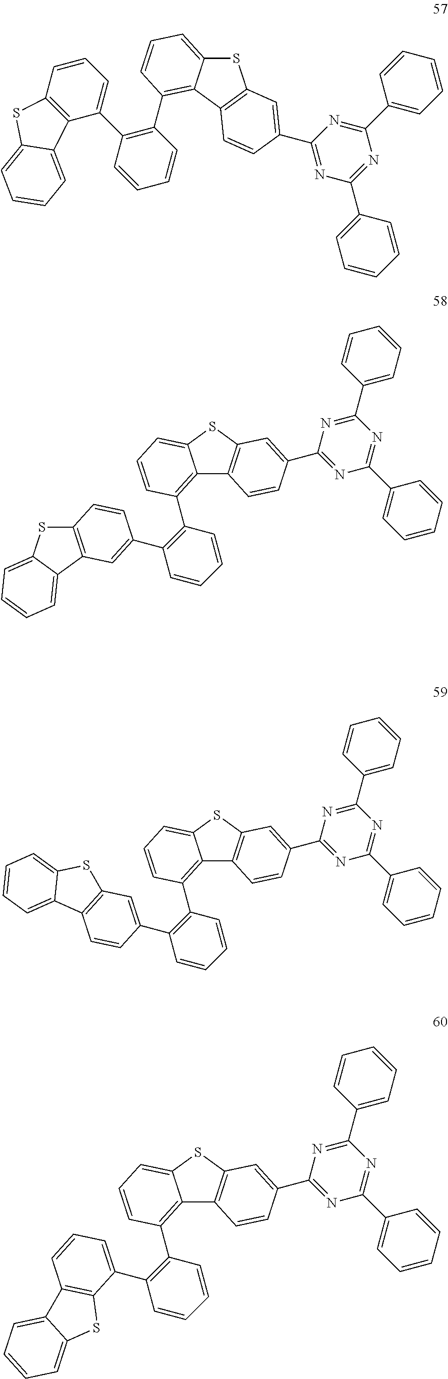 [SMILES: c1ccc(-c2nc(-c3ccccc3)nc(-c3ccc4c(c3)sc3cccc(-c5ccccc5-c5cccc6sc7ccccc7c56)c34)n2)cc1 ; c1ccc(-c2nc(-c3ccccc3)nc(-c3ccc4c(c3)sc3cccc(-c5ccccc5-c5ccc6sc7ccccc7c6c5)c34)n2)cc1 ; c1ccc(-c2nc(-c3ccccc3)nc(-c3ccc4c(c3)sc3cccc(-c5ccccc5-c5ccc6c(c5)sc5ccccc56)c34)n2)cc1 ; c1ccc(-c2nc(-c3ccccc3)nc(-c3ccc4c(c3)sc3cccc(-c5ccccc5-c5cccc6c5sc5ccccc56)c34)n2)cc1]

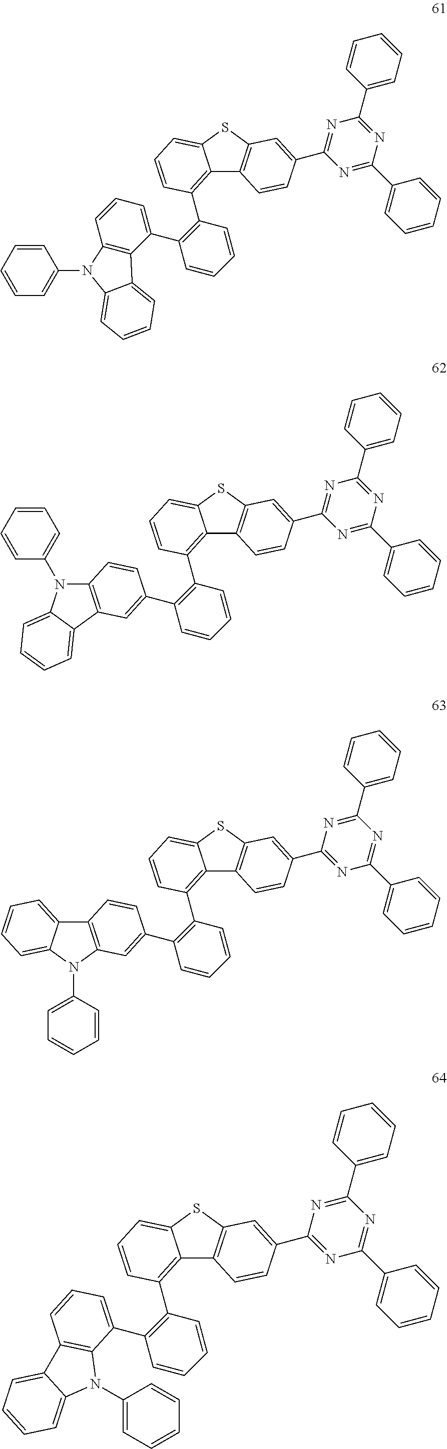 [SMILES: c1ccc(-c2nc(-c3ccccc3)nc(-c3ccc4c(c3)sc3cccc(-c5ccccc5-c5cccc6c5c5ccccc5n6-c5ccccc5)c34)n2)cc1 ; c1ccc(-c2nc(-c3ccccc3)nc(-c3ccc4c(c3)sc3cccc(-c5ccccc5-c5ccc6c(c5)c5ccccc5n6-c5ccccc5)c34)n2)cc1 ; c1ccc(-c2nc(-c3ccccc3)nc(-c3ccc4c(c3)sc3cccc(-c5ccccc5-c5ccc6c7ccccc7n(-c7ccccc7)c6c5)c34)n2)cc1 ; c1ccc(-c2nc(-c3ccccc3)nc(-c3ccc4c(c3)sc3cccc(-c5ccccc5-c5cccc6c7ccccc7n(-c7ccccc7)c56)c34)n2)cc1]

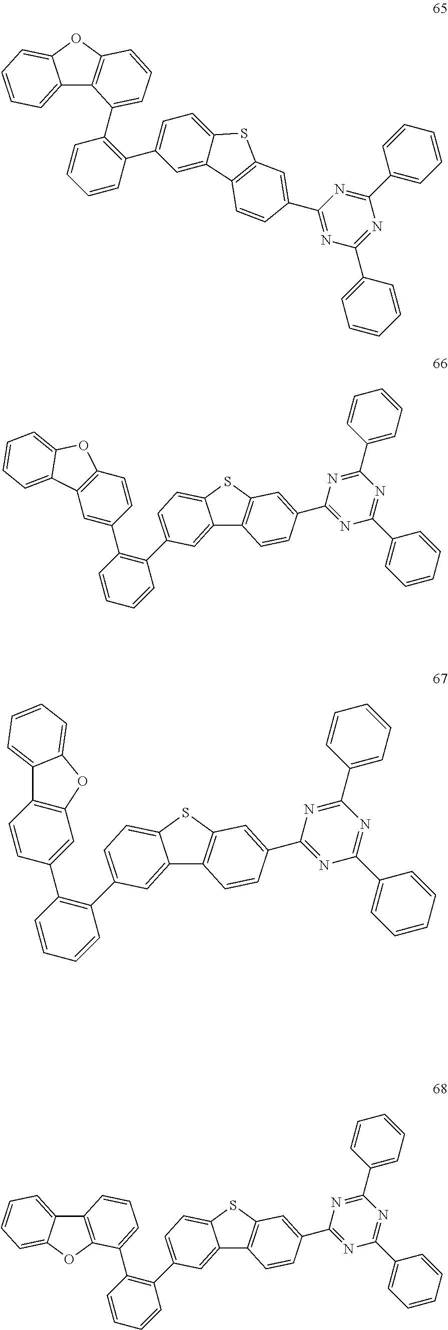 [SMILES: c1ccc(-c2nc(-c3ccccc3)nc(-c3ccc4c(c3)sc3ccc(-c5ccccc5-c5cccc6oc7ccccc7c56)cc34)n2)cc1 ; c1ccc(-c2nc(-c3ccccc3)nc(-c3ccc4c(c3)sc3ccc(-c5ccccc5-c5ccc6oc7ccccc7c6c5)cc34)n2)cc1 ; c1ccc(-c2nc(-c3ccccc3)nc(-c3ccc4c(c3)sc3ccc(-c5ccccc5-c5ccc6c(c5)oc5ccccc56)cc34)n2)cc1 ; c1ccc(-c2nc(-c3ccccc3)nc(-c3ccc4c(c3)sc3ccc(-c5ccccc5-c5cccc6c5oc5ccccc56)cc34)n2)cc1]

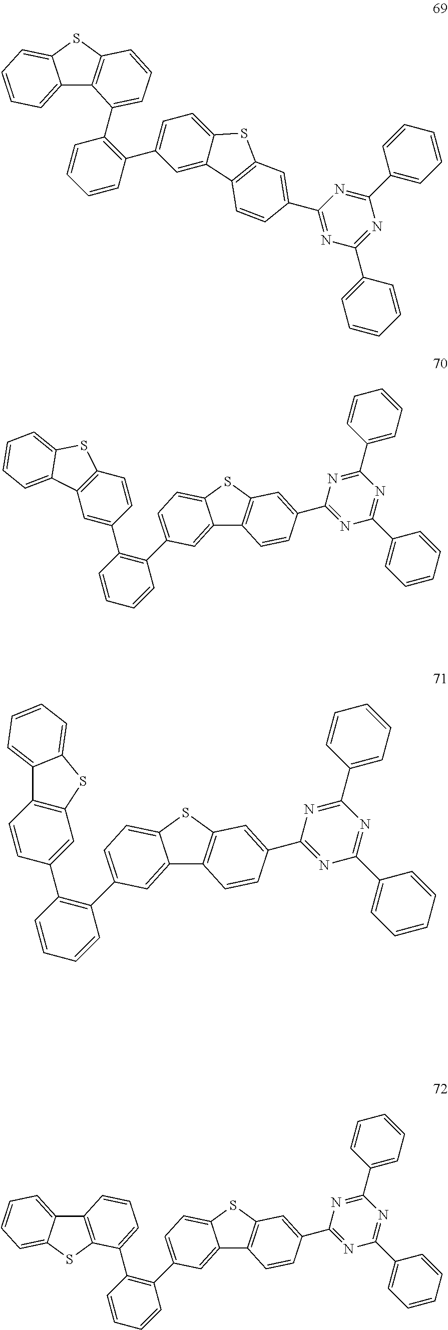 [SMILES: c1ccc(-c2nc(-c3ccccc3)nc(-c3ccc4c(c3)sc3ccc(-c5ccccc5-c5cccc6sc7ccccc7c56)cc34)n2)cc1 ; c1ccc(-c2nc(-c3ccccc3)nc(-c3ccc4c(c3)sc3ccc(-c5ccccc5-c5ccc6sc7ccccc7c6c5)cc34)n2)cc1 ; c1ccc(-c2nc(-c3ccccc3)nc(-c3ccc4c(c3)sc3ccc(-c5ccccc5-c5ccc6c(c5)sc5ccccc56)cc34)n2)cc1 ; c1ccc(-c2nc(-c3ccccc3)nc(-c3ccc4c(c3)sc3ccc(-c5ccccc5-c5cccc6c5sc5ccccc56)cc34)n2)cc1]

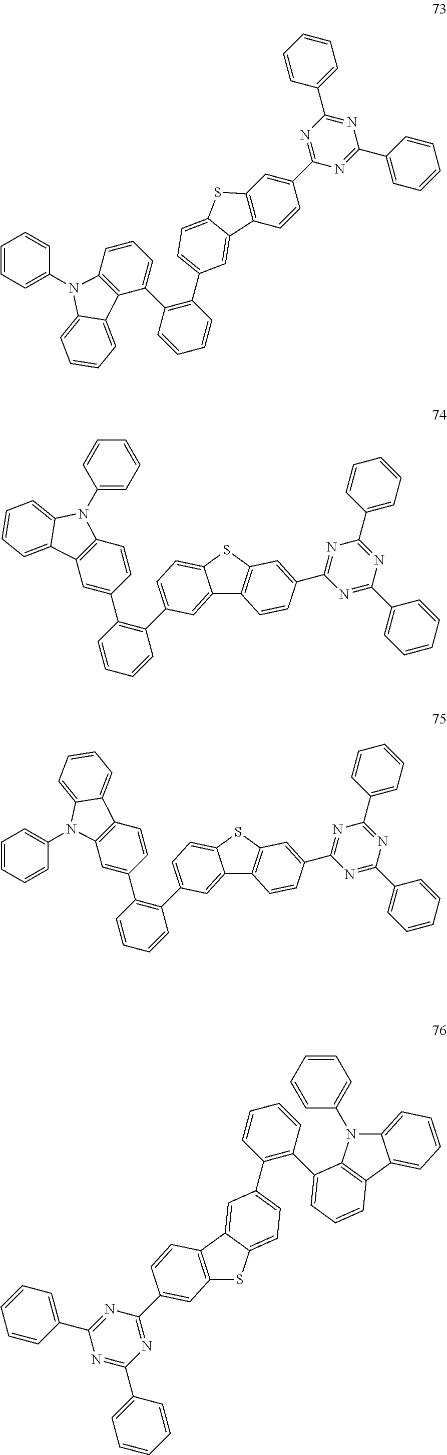 [SMILES: c1ccc(-c2nc(-c3ccccc3)nc(-c3ccc4c(c3)sc3ccc(-c5ccccc5-c5cccc6c5c5ccccc5n6-c5ccccc5)cc34)n2)cc1 ; c1ccc(-c2nc(-c3ccccc3)nc(-c3ccc4c(c3)sc3ccc(-c5ccccc5-c5ccc6c(c5)c5ccccc5n6-c5ccccc5)cc34)n2)cc1 ; c1ccc(-c2nc(-c3ccccc3)nc(-c3ccc4c(c3)sc3ccc(-c5ccccc5-c5ccc6c7ccccc7n(-c7ccccc7)c6c5)cc34)n2)cc1 ; c1ccc(-c2nc(-c3ccccc3)nc(-c3ccc4c(c3)sc3ccc(-c5ccccc5-c5cccc6c7ccccc7n(-c7ccccc7)c56)cc34)n2)cc1]

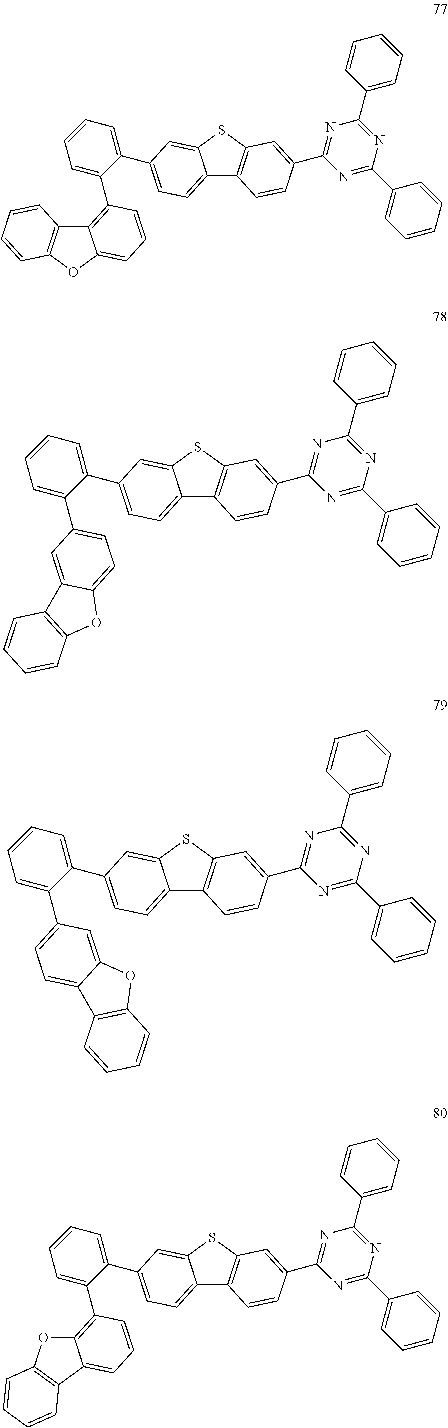 [SMILES: c1ccc(-c2nc(-c3ccccc3)nc(-c3ccc4c(c3)sc3cc(-c5ccccc5-c5cccc6oc7ccccc7c56)ccc34)n2)cc1 ; c1ccc(-c2nc(-c3ccccc3)nc(-c3ccc4c(c3)sc3cc(-c5ccccc5-c5ccc6oc7ccccc7c6c5)ccc34)n2)cc1 ; c1ccc(-c2nc(-c3ccccc3)nc(-c3ccc4c(c3)sc3cc(-c5ccccc5-c5ccc6c(c5)oc5ccccc56)ccc34)n2)cc1 ; c1ccc(-c2nc(-c3ccccc3)nc(-c3ccc4c(c3)sc3cc(-c5ccccc5-c5cccc6c5oc5ccccc56)ccc34)n2)cc1]

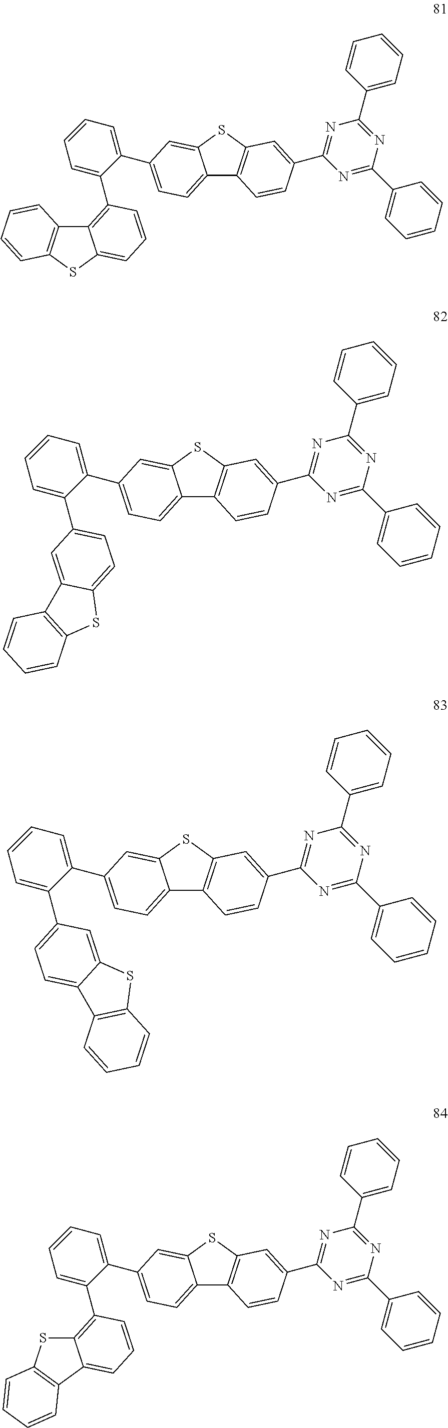 [SMILES: c1ccc(-c2nc(-c3ccccc3)nc(-c3ccc4c(c3)sc3cc(-c5ccccc5-c5cccc6sc7ccccc7c56)ccc34)n2)cc1 ; c1ccc(-c2nc(-c3ccccc3)nc(-c3ccc4c(c3)sc3cc(-c5ccccc5-c5ccc6sc7ccccc7c6c5)ccc34)n2)cc1 ; c1ccc(-c2nc(-c3ccccc3)nc(-c3ccc4c(c3)sc3cc(-c5ccccc5-c5ccc6c(c5)sc5ccccc56)ccc34)n2)cc1 ; c1ccc(-c2nc(-c3ccccc3)nc(-c3ccc4c(c3)sc3cc(-c5ccccc5-c5cccc6c5sc5ccccc56)ccc34)n2)cc1]

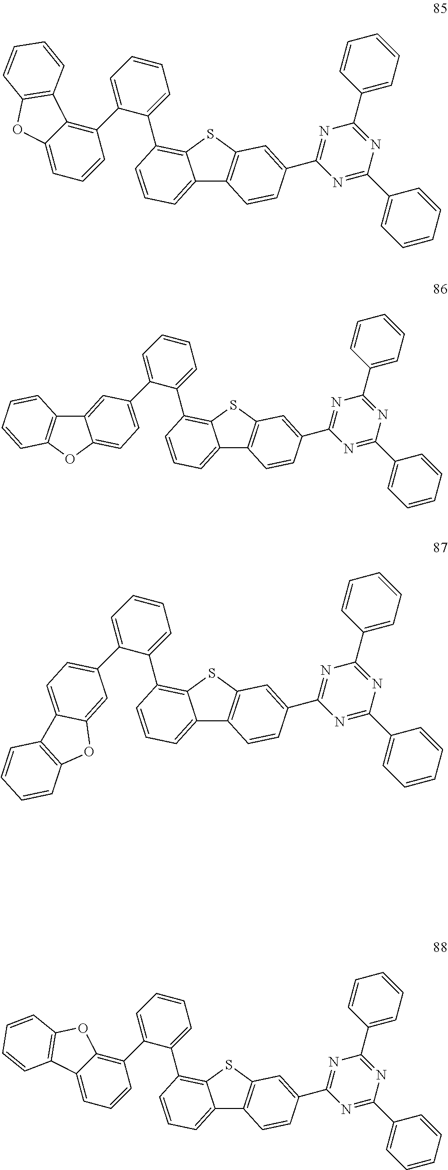 [SMILES: c1ccc(-c2nc(-c3ccccc3)nc(-c3ccc4c(c3)sc3c(-c5ccccc5-c5cccc6oc7ccccc7c56)cccc34)n2)cc1 ; c1ccc(-c2nc(-c3ccccc3)nc(-c3ccc4c(c3)sc3c(-c5ccccc5-c5ccc6oc7ccccc7c6c5)cccc34)n2)cc1 ; c1ccc(-c2nc(-c3ccccc3)nc(-c3ccc4c(c3)sc3c(-c5ccccc5-c5ccc6c(c5)oc5ccccc56)cccc34)n2)cc1 ; c1ccc(-c2nc(-c3ccccc3)nc(-c3ccc4c(c3)sc3c(-c5ccccc5-c5cccc6c5oc5ccccc56)cccc34)n2)cc1]

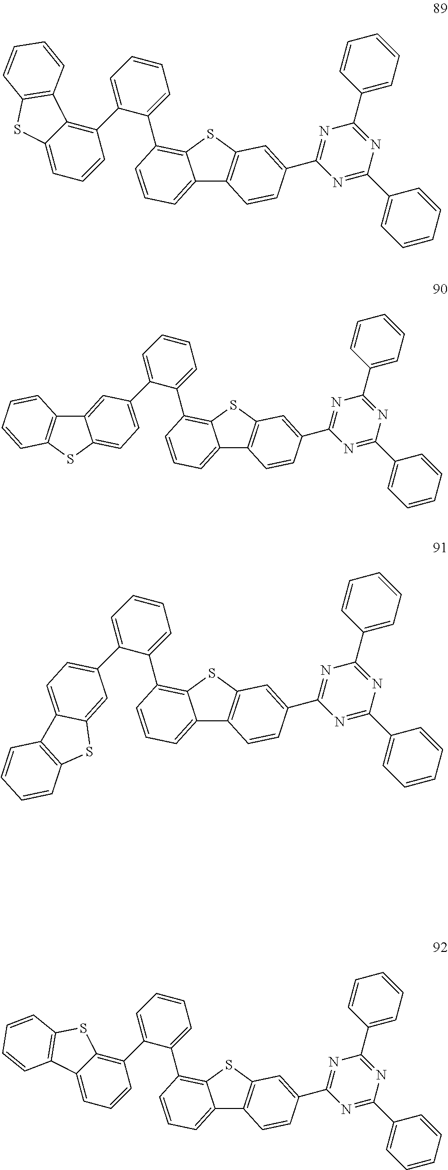 [SMILES: c1ccc(-c2nc(-c3ccccc3)nc(-c3ccc4c(c3)sc3c(-c5ccccc5-c5cccc6sc7ccccc7c56)cccc34)n2)cc1 ; c1ccc(-c2nc(-c3ccccc3)nc(-c3ccc4c(c3)sc3c(-c5ccccc5-c5ccc6sc7ccccc7c6c5)cccc34)n2)cc1 ; c1ccc(-c2nc(-c3ccccc3)nc(-c3ccc4c(c3)sc3c(-c5ccccc5-c5ccc6c(c5)sc5ccccc56)cccc34)n2)cc1 ; c1ccc(-c2nc(-c3ccccc3)nc(-c3ccc4c(c3)sc3c(-c5ccccc5-c5cccc6c5sc5ccccc56)cccc34)n2)cc1]

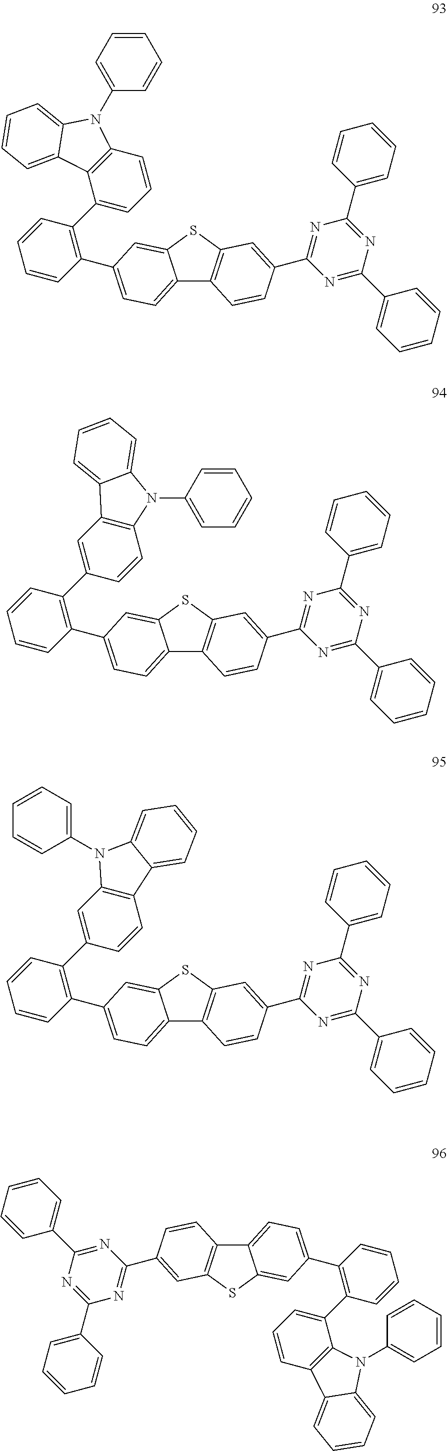 [SMILES: c1ccc(-c2nc(-c3ccccc3)nc(-c3ccc4c(c3)sc3cc(-c5ccccc5-c5cccc6c5c5ccccc5n6-c5ccccc5)ccc34)n2)cc1 ; c1ccc(-c2nc(-c3ccccc3)nc(-c3ccc4c(c3)sc3cc(-c5ccccc5-c5ccc6c(c5)c5ccccc5n6-c5ccccc5)ccc34)n2)cc1 ; c1ccc(-c2nc(-c3ccccc3)nc(-c3ccc4c(c3)sc3cc(-c5ccccc5-c5ccc6c7ccccc7n(-c7ccccc7)c6c5)ccc34)n2)cc1 ; c1ccc(-c2nc(-c3ccccc3)nc(-c3ccc4c(c3)sc3cc(-c5ccccc5-c5cccc6c7ccccc7n(-c7ccccc7)c56)ccc34)n2)cc1]

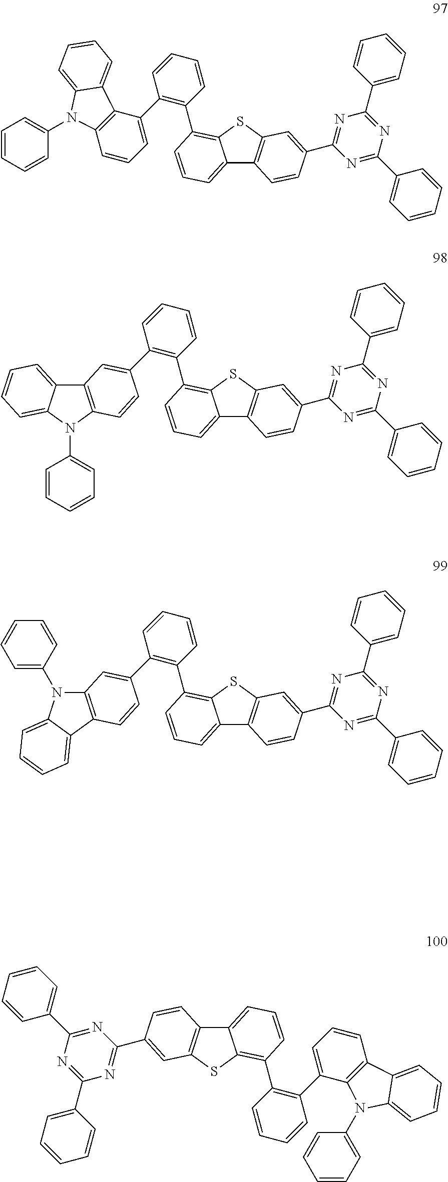 [SMILES: c1ccc(-c2nc(-c3ccccc3)nc(-c3ccc4c(c3)sc3c(-c5ccccc5-c5cccc6c5c5ccccc5n6-c5ccccc5)cccc34)n2)cc1 ; c1ccc(-c2nc(-c3ccccc3)nc(-c3ccc4c(c3)sc3c(-c5ccccc5-c5ccc6c(c5)c5ccccc5n6-c5ccccc5)cccc34)n2)cc1 ; c1ccc(-c2nc(-c3ccccc3)nc(-c3ccc4c(c3)sc3c(-c5ccccc5-c5ccc6c7ccccc7n(-c7ccccc7)c6c5)cccc34)n2)cc1 ; c1ccc(-c2nc(-c3ccccc3)nc(-c3ccc4c(c3)sc3c(-c5ccccc5-c5cccc6c7ccccc7n(-c7ccccc7)c56)cccc34)n2)cc1]

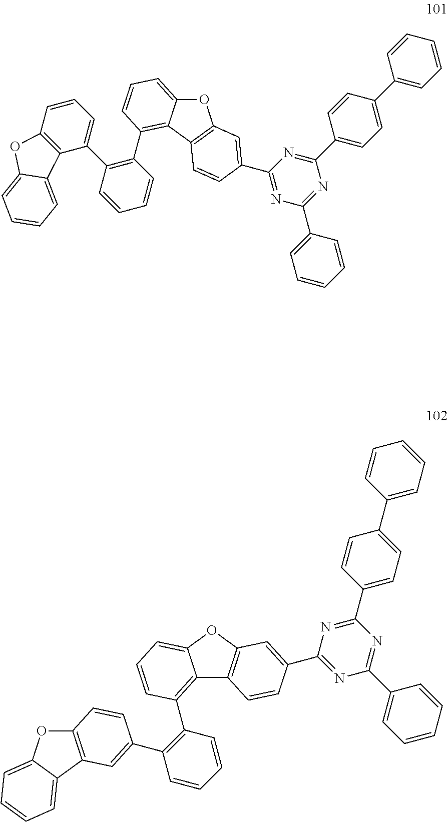 [SMILES: c1ccc(-c2ccc(-c3nc(-c4ccccc4)nc(-c4ccc5c(c4)oc4cccc(-c6ccccc6-c6cccc7oc8ccccc8c67)c45)n3)cc2)cc1 ; c1ccc(-c2ccc(-c3nc(-c4ccccc4)nc(-c4ccc5c(c4)oc4cccc(-c6ccccc6-c6ccc7oc8ccccc8c7c6)c45)n3)cc2)cc1]

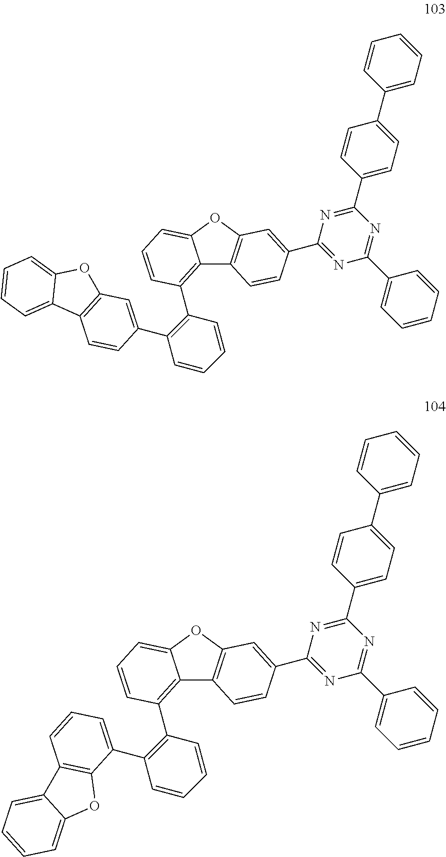 [SMILES: c1ccc(-c2ccc(-c3nc(-c4ccccc4)nc(-c4ccc5c(c4)oc4cccc(-c6ccccc6-c6ccc7c(c6)oc6ccccc67)c45)n3)cc2)cc1 ; c1ccc(-c2ccc(-c3nc(-c4ccccc4)nc(-c4ccc5c(c4)oc4cccc(-c6ccccc6-c6cccc7c6oc6ccccc67)c45)n3)cc2)cc1]

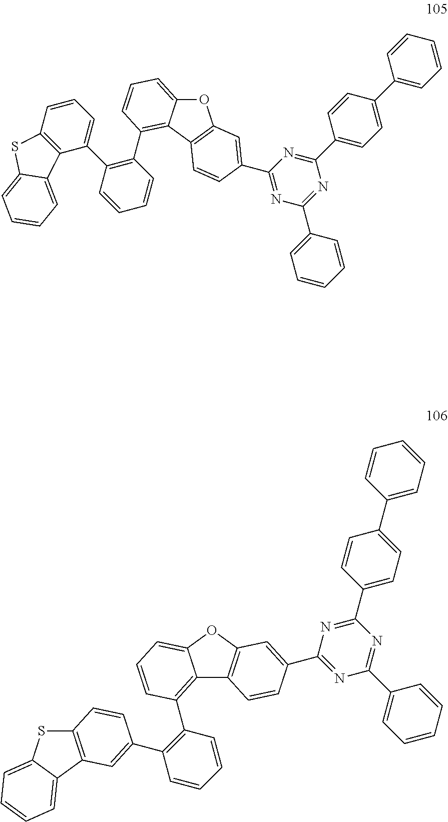 [SMILES: c1ccc(-c2ccc(-c3nc(-c4ccccc4)nc(-c4ccc5c(c4)oc4cccc(-c6ccccc6-c6cccc7sc8ccccc8c67)c45)n3)cc2)cc1 ; c1ccc(-c2ccc(-c3nc(-c4ccccc4)nc(-c4ccc5c(c4)oc4cccc(-c6ccccc6-c6ccc7sc8ccccc8c7c6)c45)n3)cc2)cc1]

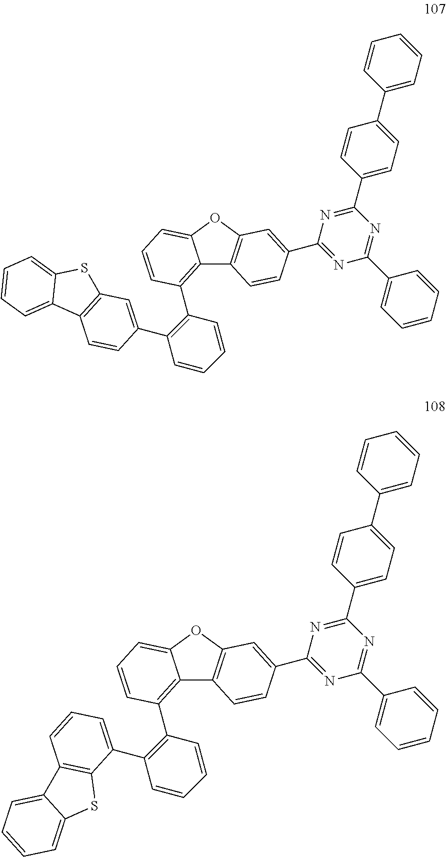 [SMILES: c1ccc(-c2ccc(-c3nc(-c4ccccc4)nc(-c4ccc5c(c4)oc4cccc(-c6ccccc6-c6ccc7c(c6)sc6ccccc67)c45)n3)cc2)cc1 ; c1ccc(-c2ccc(-c3nc(-c4ccccc4)nc(-c4ccc5c(c4)oc4cccc(-c6ccccc6-c6cccc7c6sc6ccccc67)c45)n3)cc2)cc1]

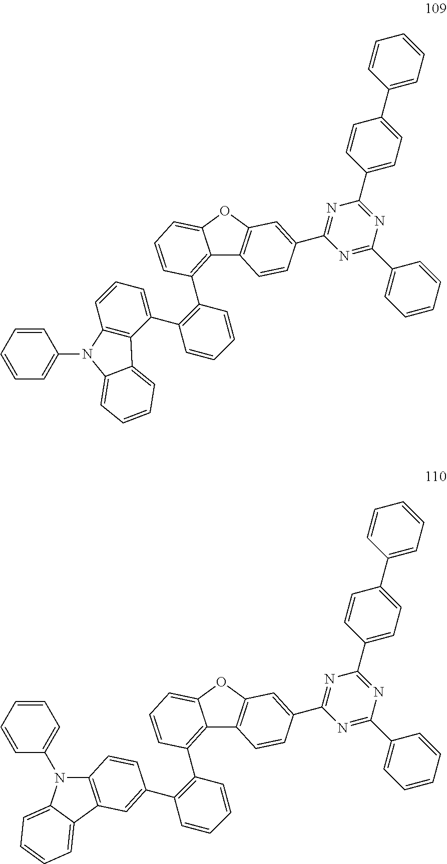 [SMILES: c1ccc(-c2ccc(-c3nc(-c4ccccc4)nc(-c4ccc5c(c4)oc4cccc(-c6ccccc6-c6cccc7c6c6ccccc6n7-c6ccccc6)c45)n3)cc2)cc1 ; c1ccc(-c2ccc(-c3nc(-c4ccccc4)nc(-c4ccc5c(c4)oc4cccc(-c6ccccc6-c6ccc7c(c6)c6ccccc6n7-c6ccccc6)c45)n3)cc2)cc1]

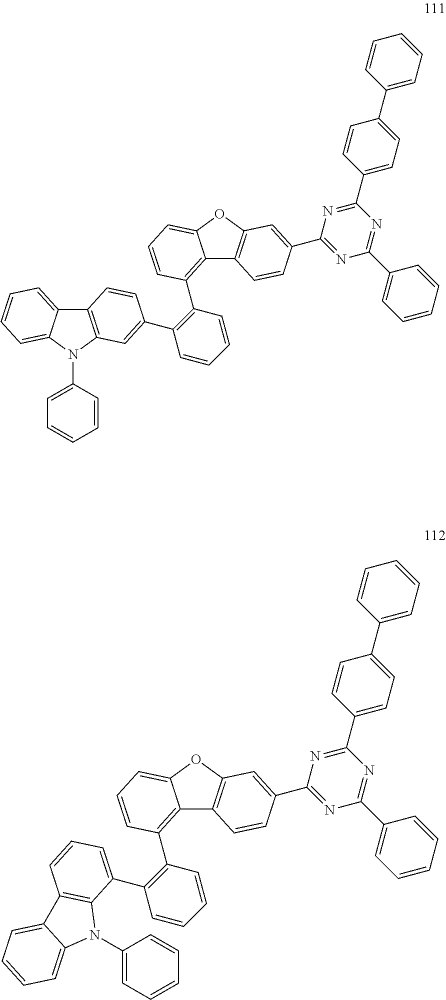 [SMILES: c1ccc(-c2ccc(-c3nc(-c4ccccc4)nc(-c4ccc5c(c4)oc4cccc(-c6ccccc6-c6ccc7c8ccccc8n(-c8ccccc8)c7c6)c45)n3)cc2)cc1 ; c1ccc(-c2ccc(-c3nc(-c4ccccc4)nc(-c4ccc5c(c4)oc4cccc(-c6ccccc6-c6cccc7c8ccccc8n(-c8ccccc8)c67)c45)n3)cc2)cc1]

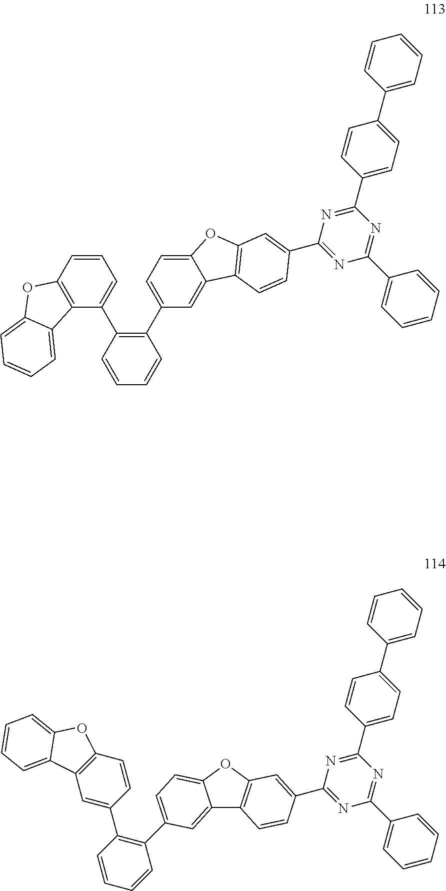 [SMILES: c1ccc(-c2ccc(-c3nc(-c4ccccc4)nc(-c4ccc5c(c4)oc4ccc(-c6ccccc6-c6cccc7oc8ccccc8c67)cc45)n3)cc2)cc1 ; c1ccc(-c2ccc(-c3nc(-c4ccccc4)nc(-c4ccc5c(c4)oc4ccc(-c6ccccc6-c6ccc7oc8ccccc8c7c6)cc45)n3)cc2)cc1]

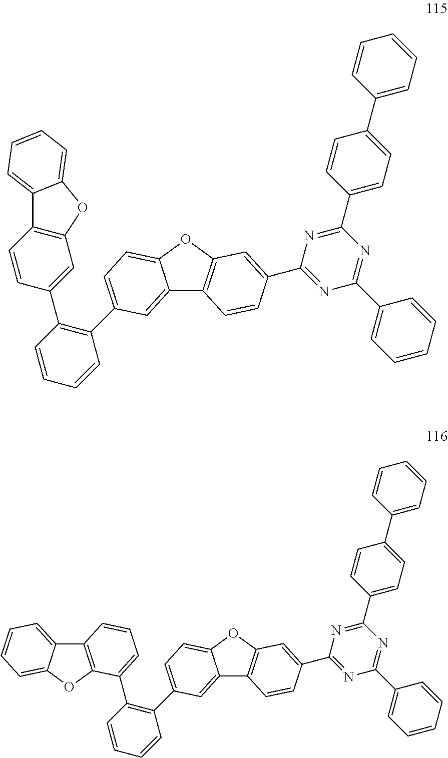 [SMILES: c1ccc(-c2ccc(-c3nc(-c4ccccc4)nc(-c4ccc5c(c4)oc4ccc(-c6ccccc6-c6ccc7c(c6)oc6ccccc67)cc45)n3)cc2)cc1 ; c1ccc(-c2ccc(-c3nc(-c4ccccc4)nc(-c4ccc5c(c4)oc4ccc(-c6ccccc6-c6cccc7c6oc6ccccc67)cc45)n3)cc2)cc1]

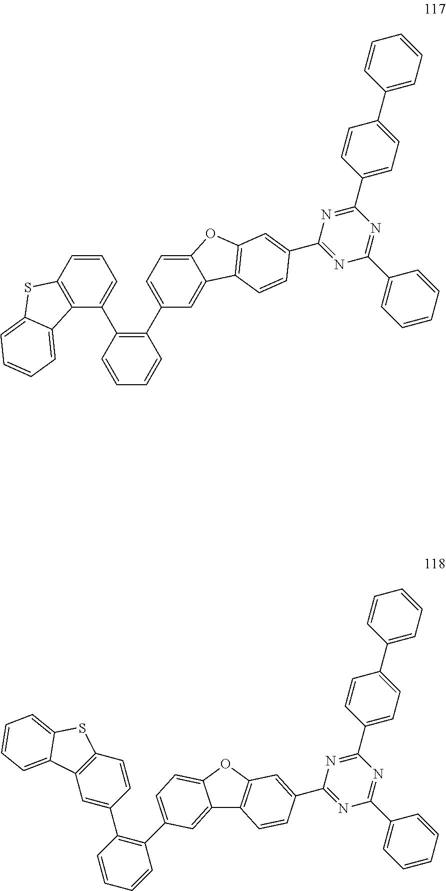 [SMILES: c1ccc(-c2ccc(-c3nc(-c4ccccc4)nc(-c4ccc5c(c4)oc4ccc(-c6ccccc6-c6cccc7sc8ccccc8c67)cc45)n3)cc2)cc1 ; c1ccc(-c2ccc(-c3nc(-c4ccccc4)nc(-c4ccc5c(c4)oc4ccc(-c6ccccc6-c6ccc7sc8ccccc8c7c6)cc45)n3)cc2)cc1]

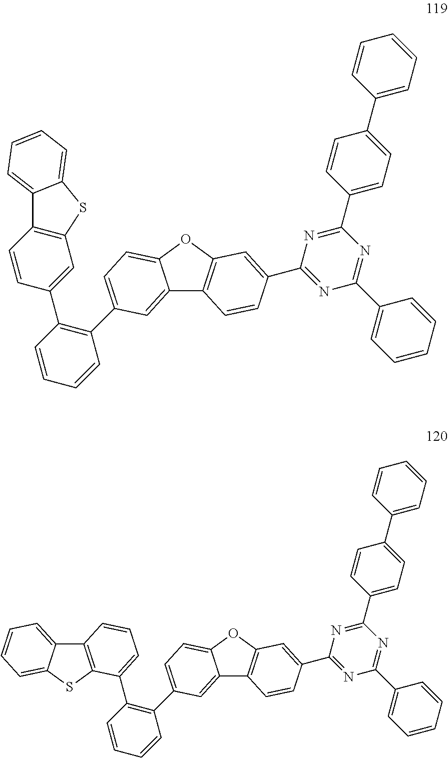 [SMILES: c1ccc(-c2ccc(-c3nc(-c4ccccc4)nc(-c4ccc5c(c4)oc4ccc(-c6ccccc6-c6ccc7c(c6)sc6ccccc67)cc45)n3)cc2)cc1 ; c1ccc(-c2ccc(-c3nc(-c4ccccc4)nc(-c4ccc5c(c4)oc4ccc(-c6ccccc6-c6cccc7c6sc6ccccc67)cc45)n3)cc2)cc1]

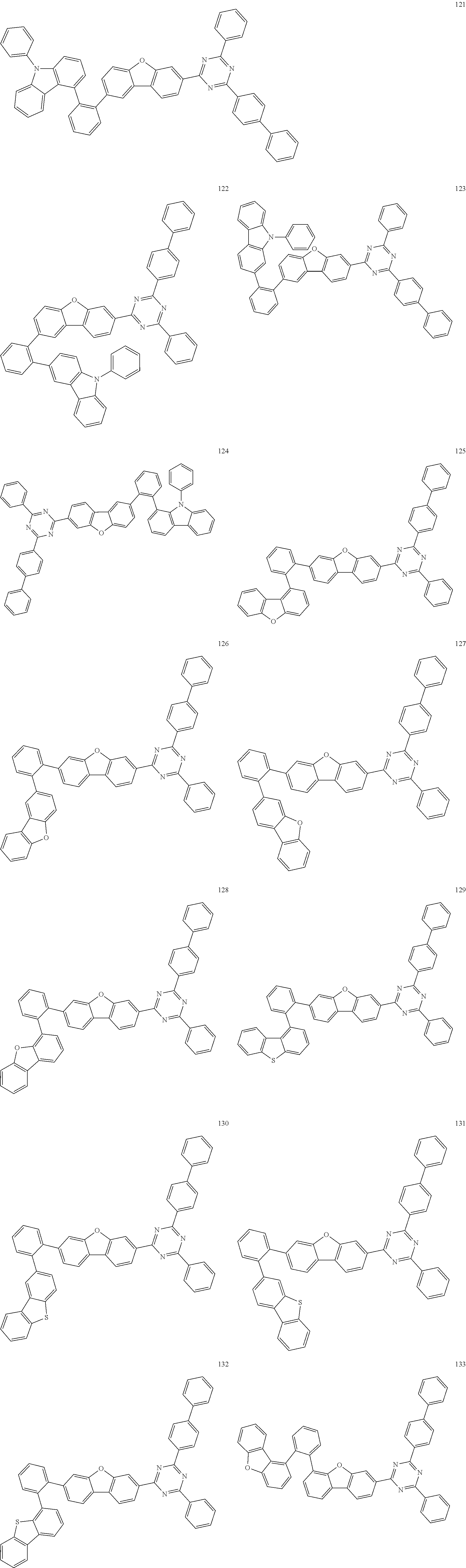

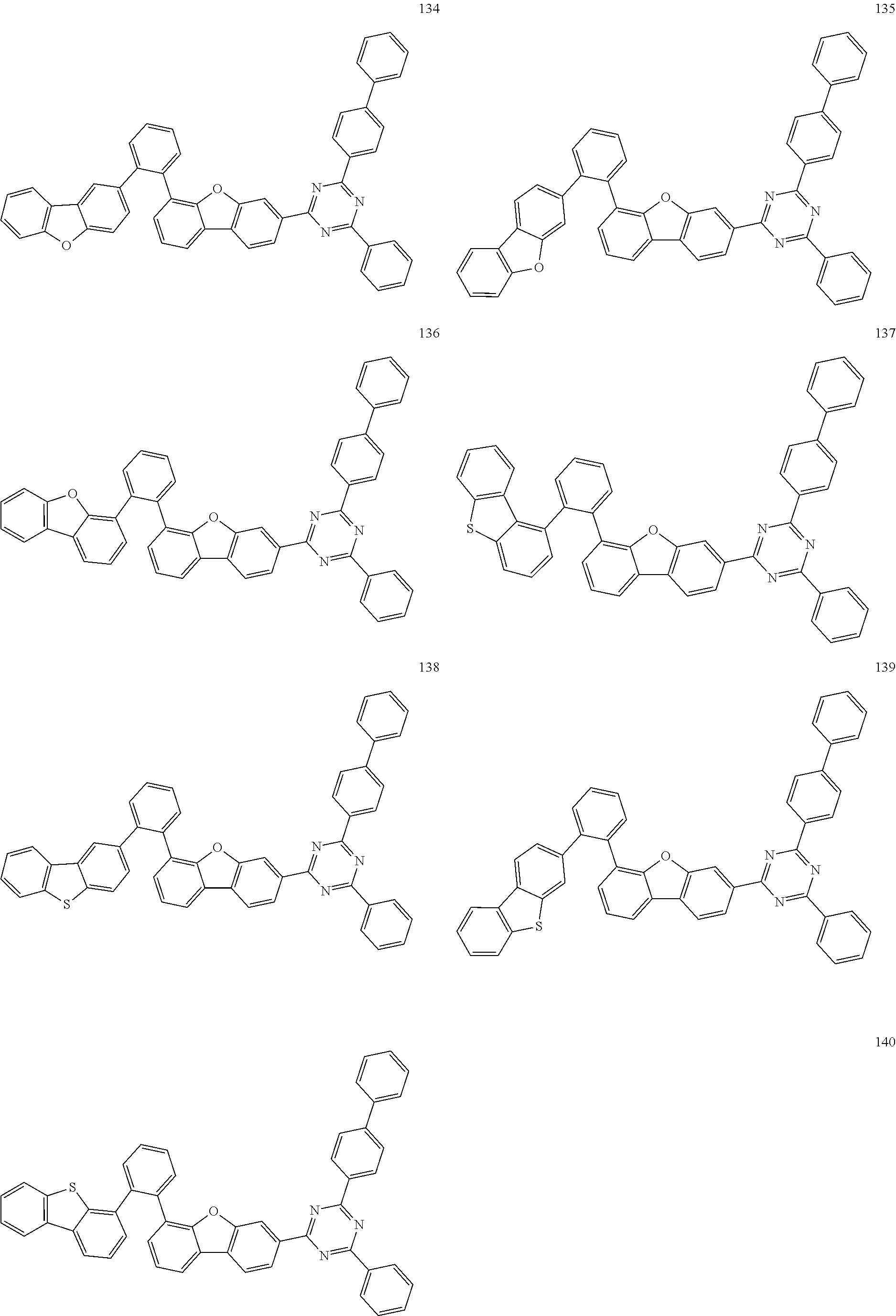

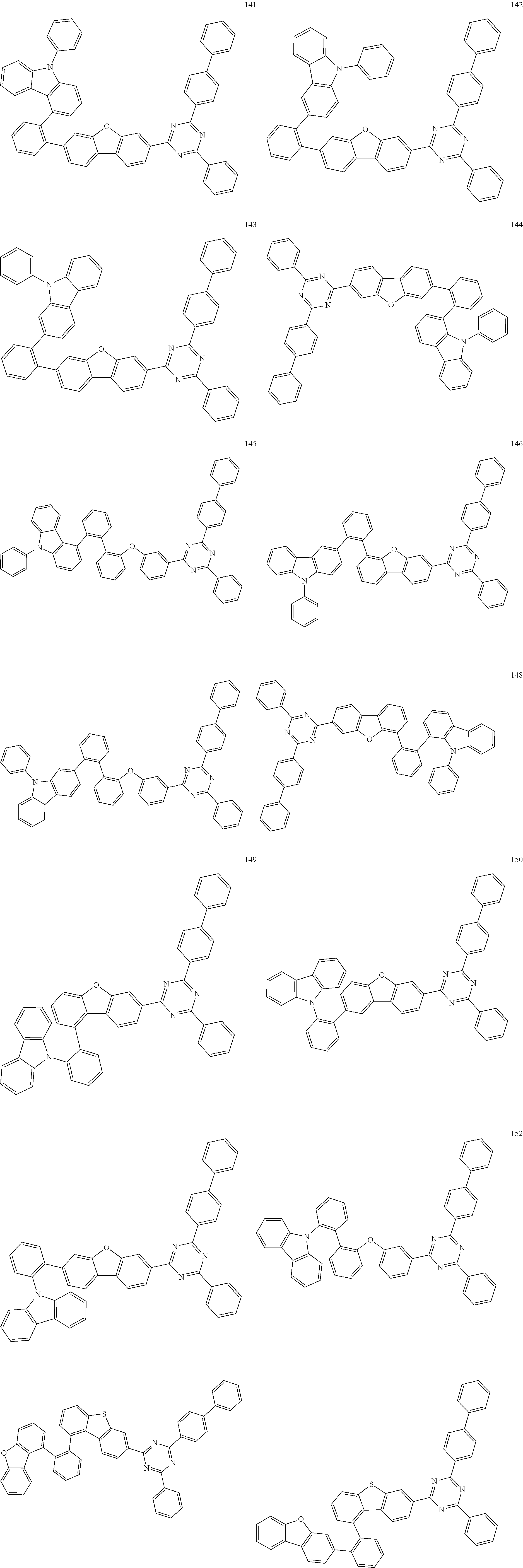

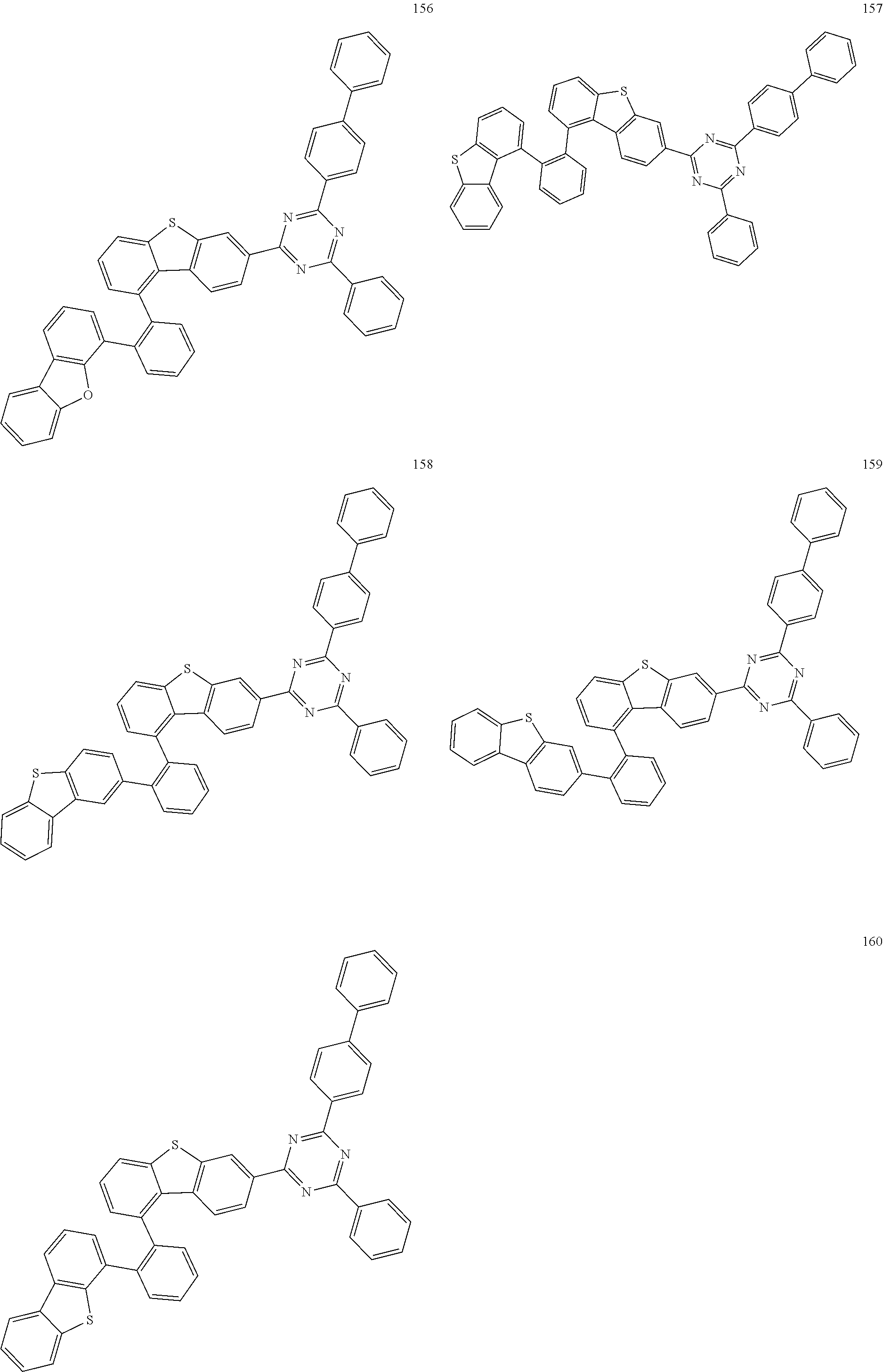 [SMILES: c1ccc(-c2ccc(-c3nc(-c4ccccc4)nc(-c4ccc5c(c4)sc4cccc(-c6ccccc6-c6cccc7c6oc6ccccc67)c45)n3)cc2)cc1 ; c1ccc(-c2ccc(-c3nc(-c4ccccc4)nc(-c4ccc5c(c4)sc4cccc(-c6ccccc6-c6cccc7sc8ccccc8c67)c45)n3)cc2)cc1 ; c1ccc(-c2ccc(-c3nc(-c4ccccc4)nc(-c4ccc5c(c4)sc4cccc(-c6ccccc6-c6ccc7sc8ccccc8c7c6)c45)n3)cc2)cc1 ; c1ccc(-c2ccc(-c3nc(-c4ccccc4)nc(-c4ccc5c(c4)sc4cccc(-c6ccccc6-c6ccc7c(c6)sc6ccccc67)c45)n3)cc2)cc1 ; c1ccc(-c2ccc(-c3nc(-c4ccccc4)nc(-c4ccc5c(c4)sc4cccc(-c6ccccc6-c6cccc7c6sc6ccccc67)c45)n3)cc2)cc1]

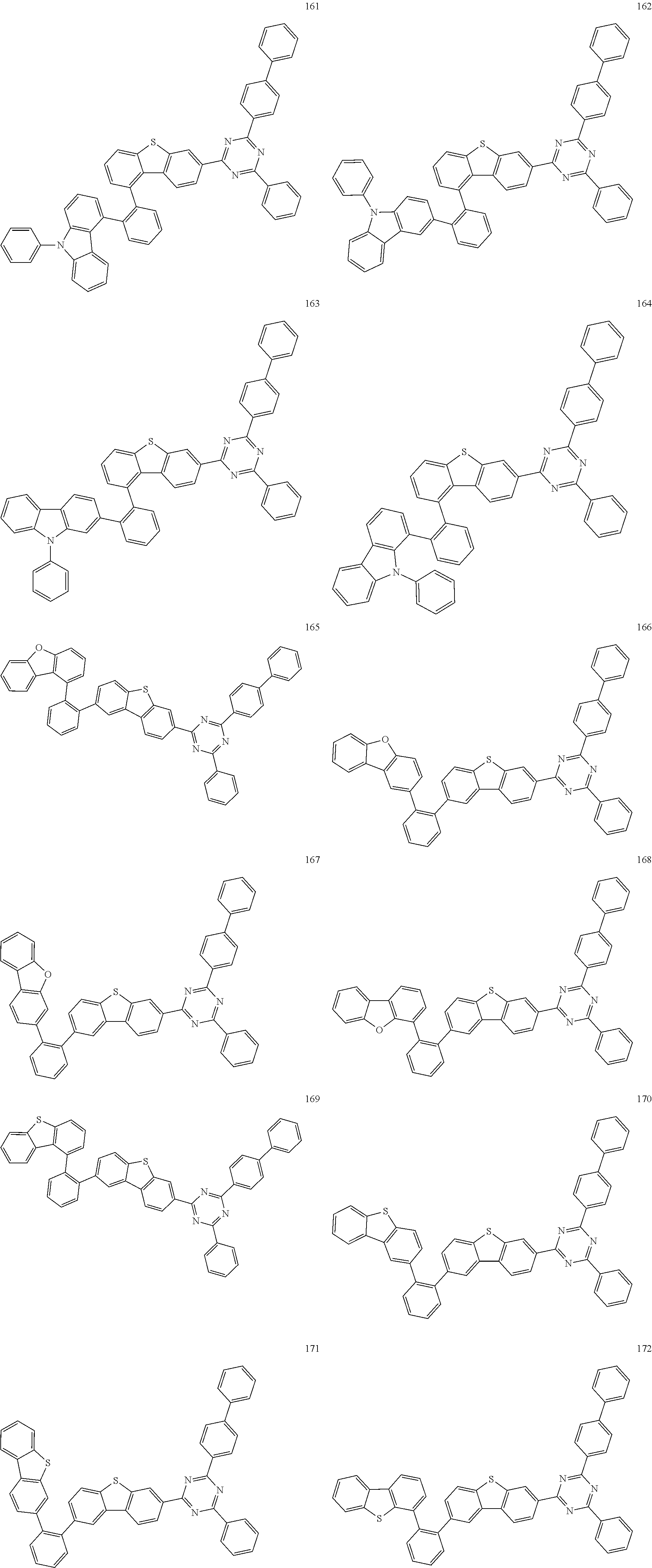

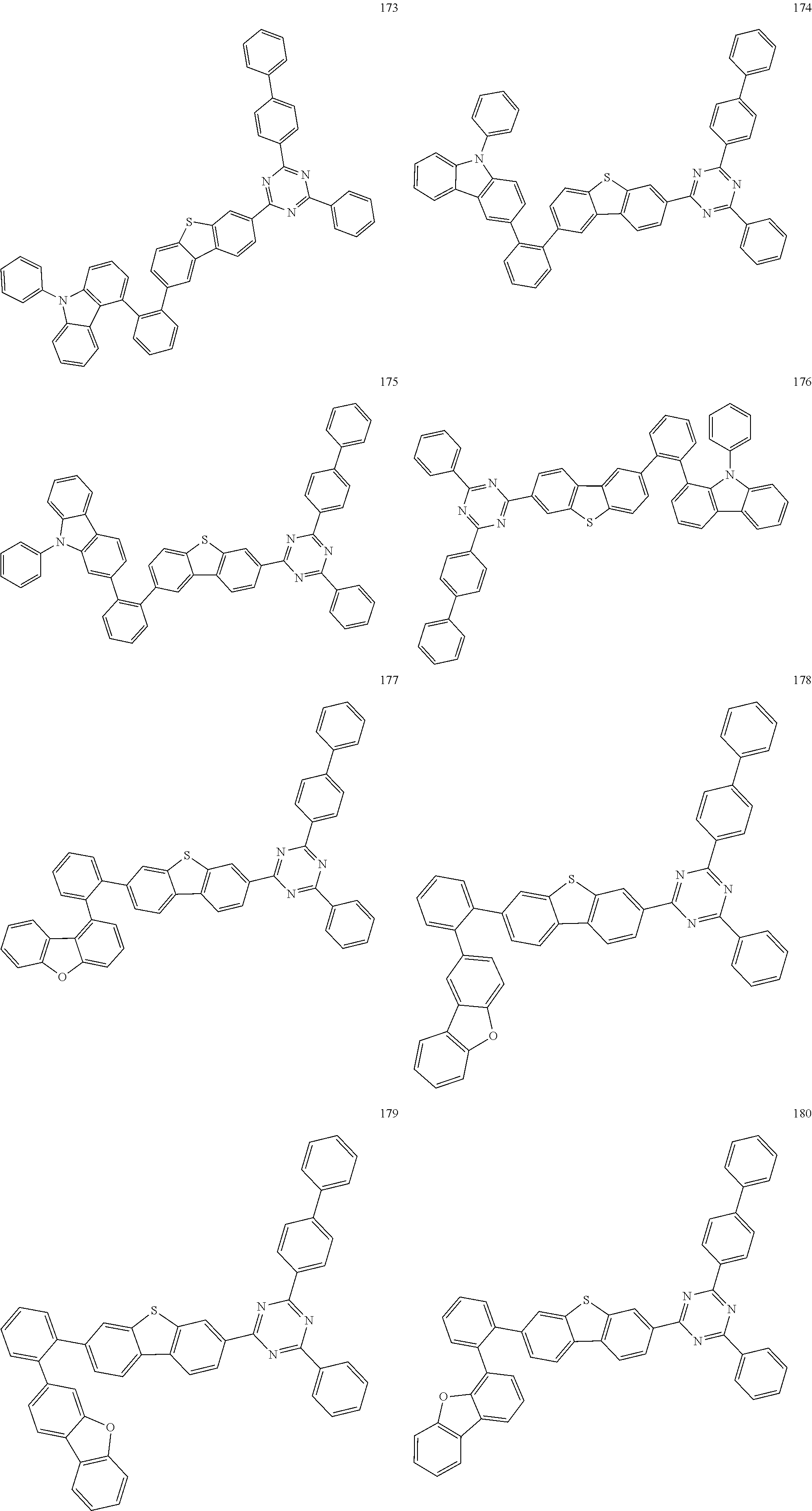

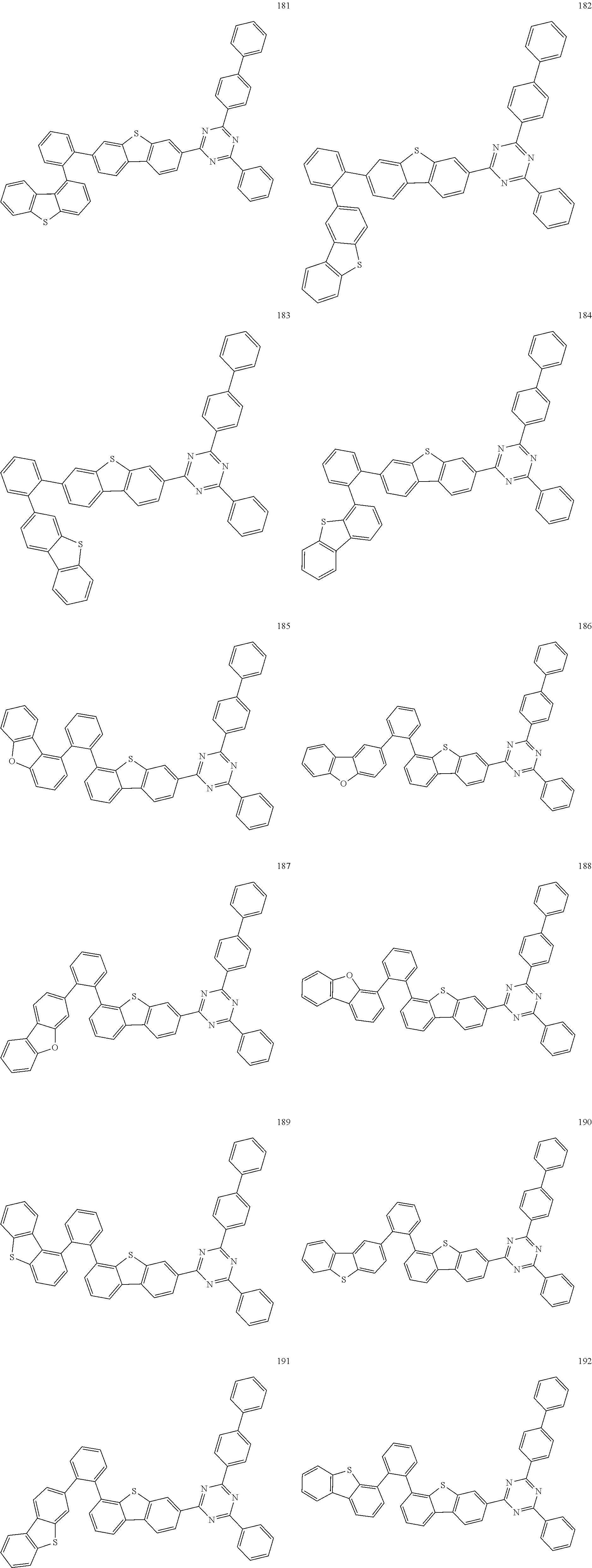

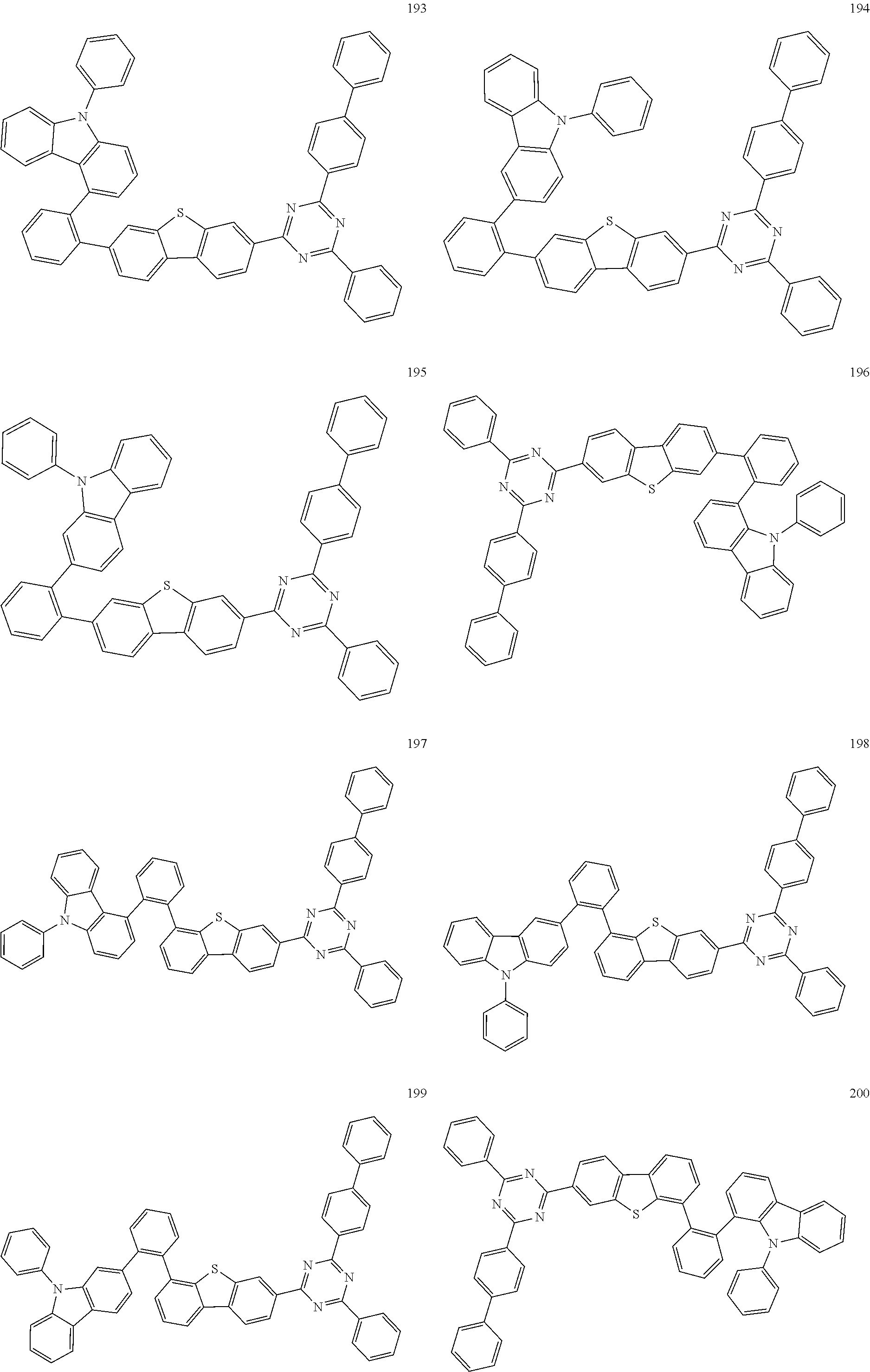

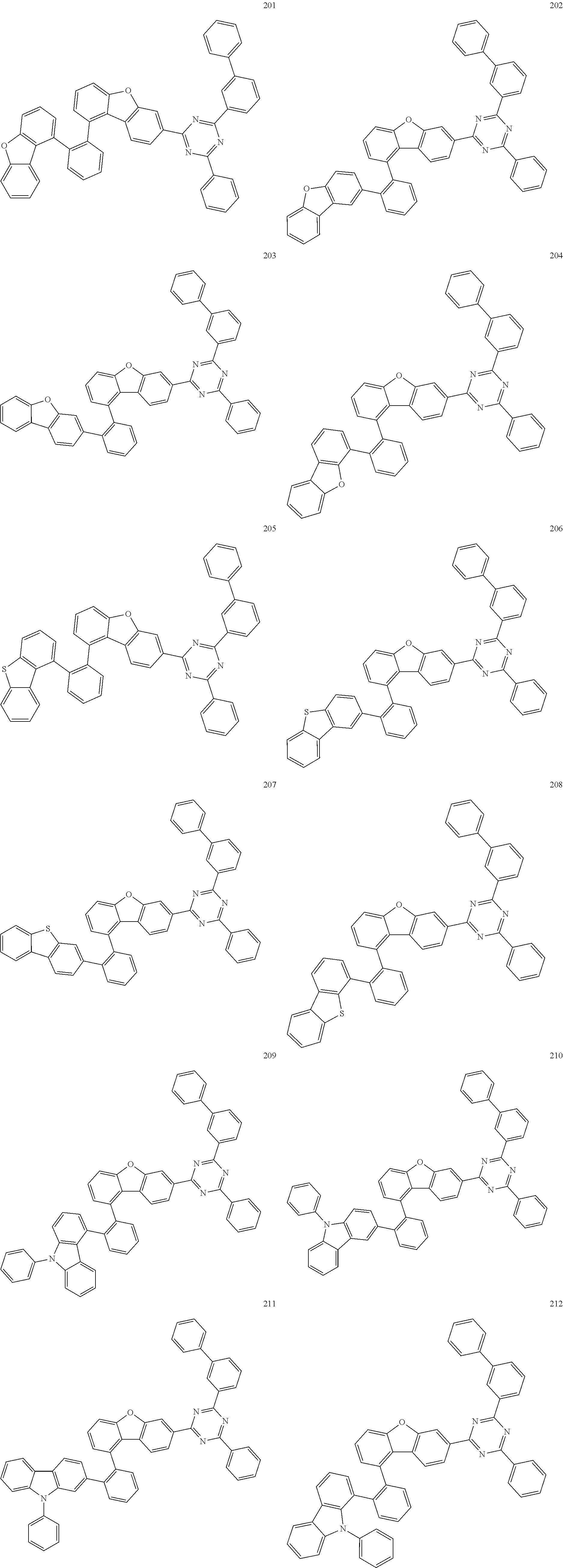

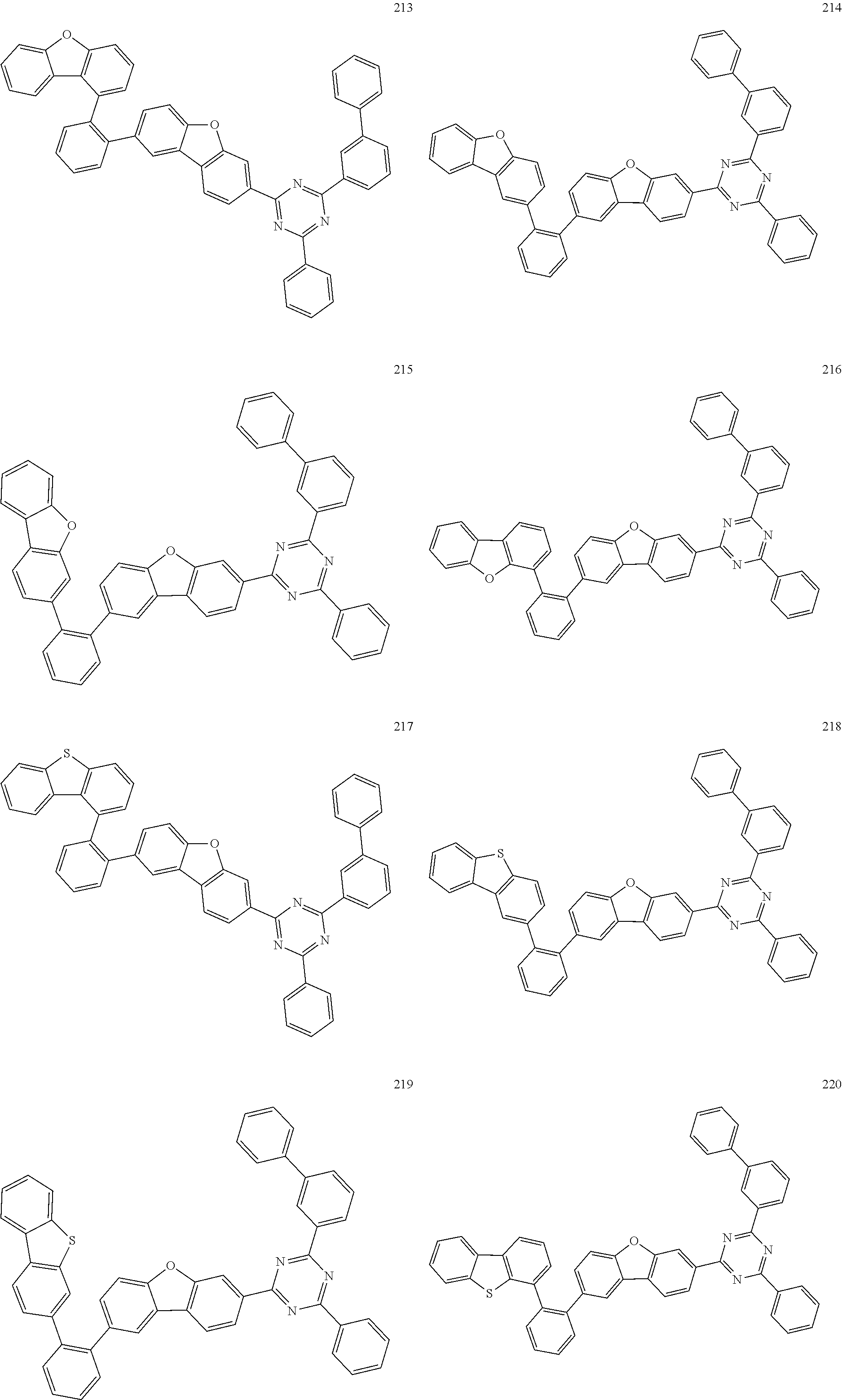

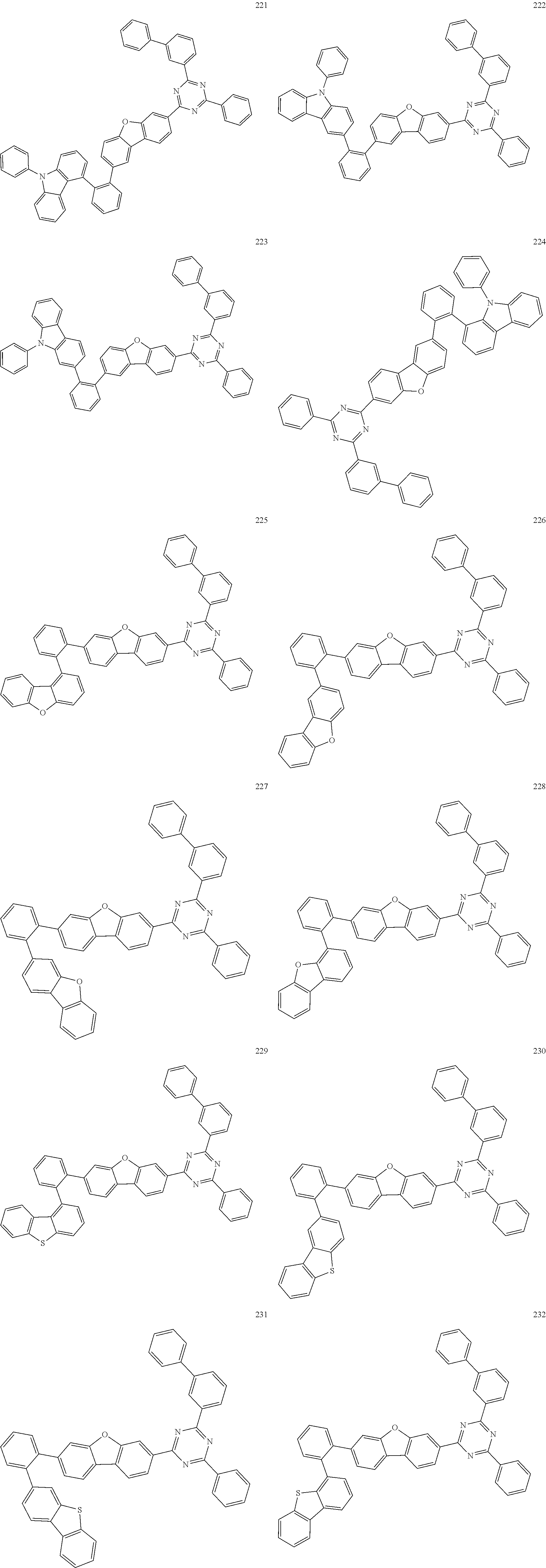

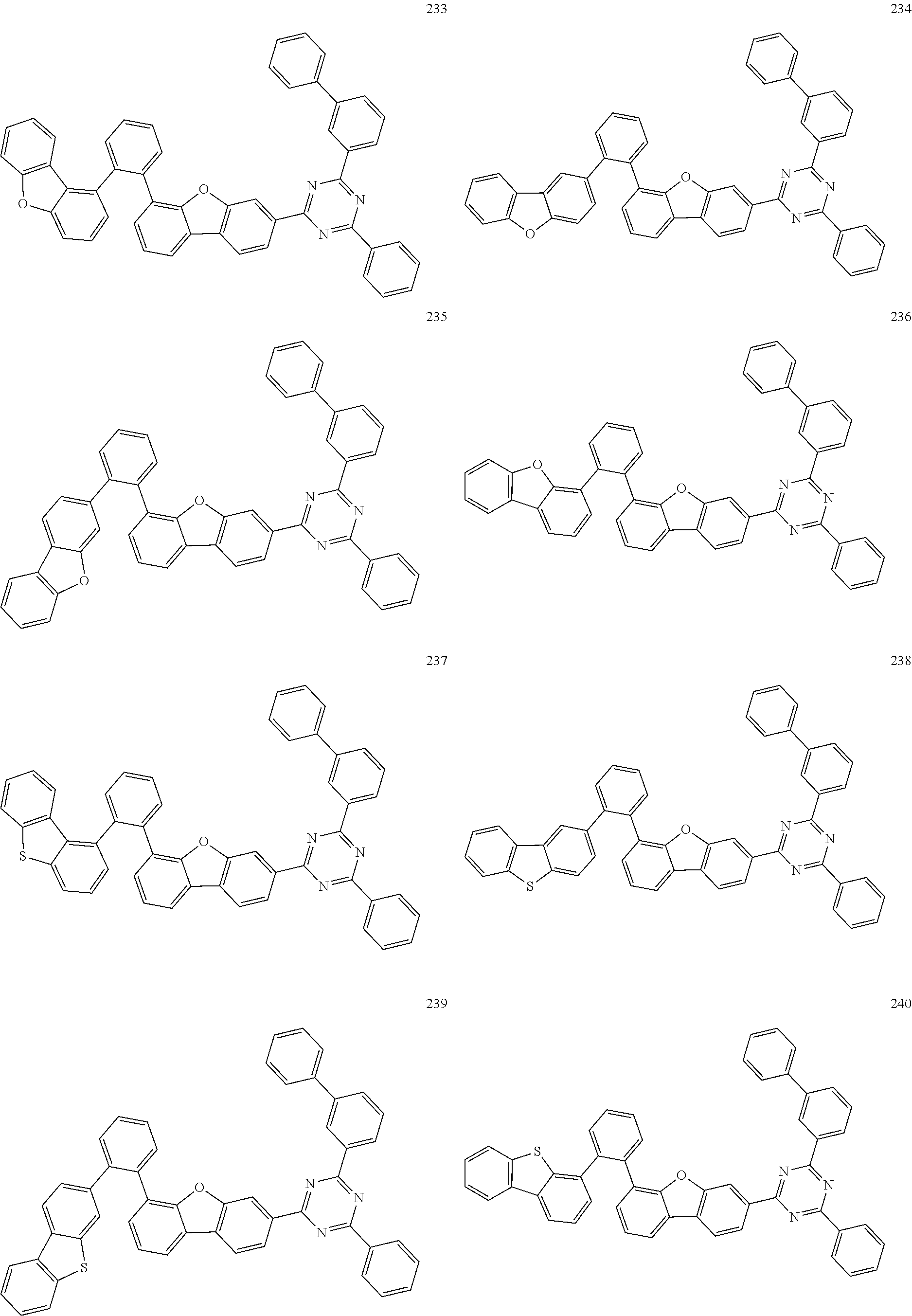

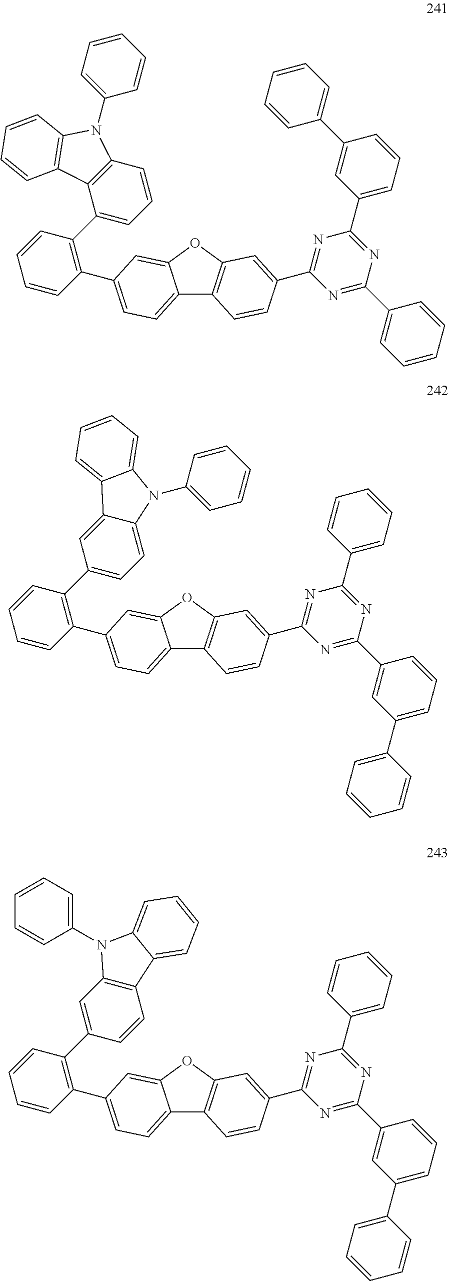 [SMILES: c1ccc(-c2cccc(-c3nc(-c4ccccc4)nc(-c4ccc5c(c4)oc4cc(-c6ccccc6-c6cccc7c6c6ccccc6n7-c6ccccc6)ccc45)n3)c2)cc1 ; c1ccc(-c2cccc(-c3nc(-c4ccccc4)nc(-c4ccc5c(c4)oc4cc(-c6ccccc6-c6ccc7c(c6)c6ccccc6n7-c6ccccc6)ccc45)n3)c2)cc1 ; c1ccc(-c2cccc(-c3nc(-c4ccccc4)nc(-c4ccc5c(c4)oc4cc(-c6ccccc6-c6ccc7c8ccccc8n(-c8ccccc8)c7c6)ccc45)n3)c2)cc1]

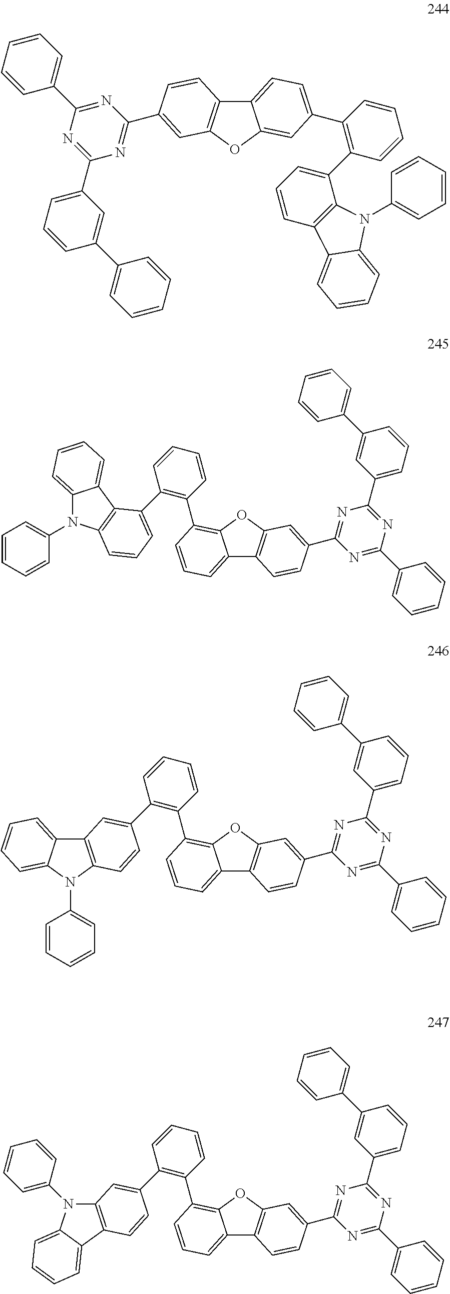 [SMILES: c1ccc(-c2cccc(-c3nc(-c4ccccc4)nc(-c4ccc5c(c4)oc4cc(-c6ccccc6-c6cccc7c8ccccc8n(-c8ccccc8)c67)ccc45)n3)c2)cc1 ; c1ccc(-c2cccc(-c3nc(-c4ccccc4)nc(-c4ccc5c(c4)oc4c(-c6ccccc6-c6cccc7c6c6ccccc6n7-c6ccccc6)cccc45)n3)c2)cc1 ; c1ccc(-c2cccc(-c3nc(-c4ccccc4)nc(-c4ccc5c(c4)oc4c(-c6ccccc6-c6ccc7c(c6)c6ccccc6n7-c6ccccc6)cccc45)n3)c2)cc1 ; c1ccc(-c2cccc(-c3nc(-c4ccccc4)nc(-c4ccc5c(c4)oc4c(-c6ccccc6-c6ccc7c8ccccc8n(-c8ccccc8)c7c6)cccc45)n3)c2)cc1]

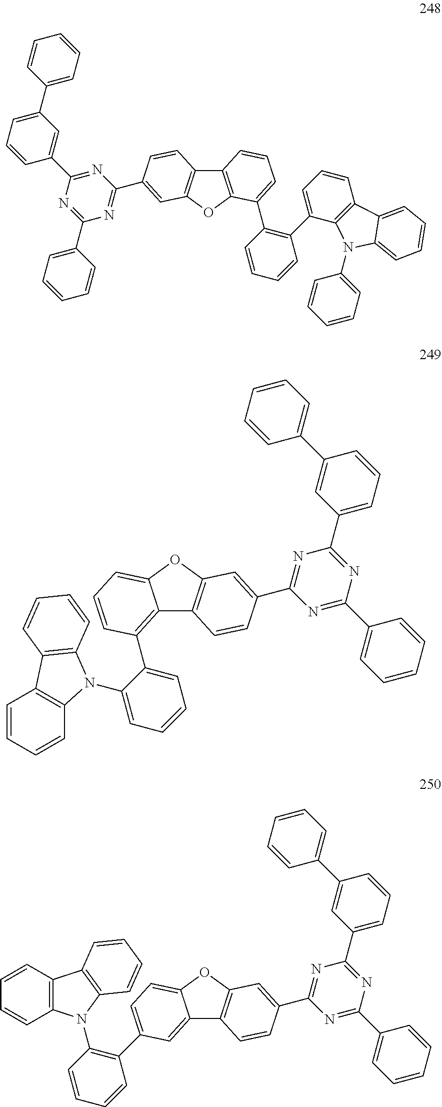 [SMILES: c1ccc(-c2cccc(-c3nc(-c4ccccc4)nc(-c4ccc5c(c4)oc4c(-c6ccccc6-c6cccc7c8ccccc8n(-c8ccccc8)c67)cccc45)n3)c2)cc1 ; c1ccc(-c2cccc(-c3nc(-c4ccccc4)nc(-c4ccc5c(c4)oc4cccc(-c6ccccc6-n6c7ccccc7c7ccccc76)c45)n3)c2)cc1 ; c1ccc(-c2cccc(-c3nc(-c4ccccc4)nc(-c4ccc5c(c4)oc4ccc(-c6ccccc6-n6c7ccccc7c7ccccc76)cc45)n3)c2)cc1]

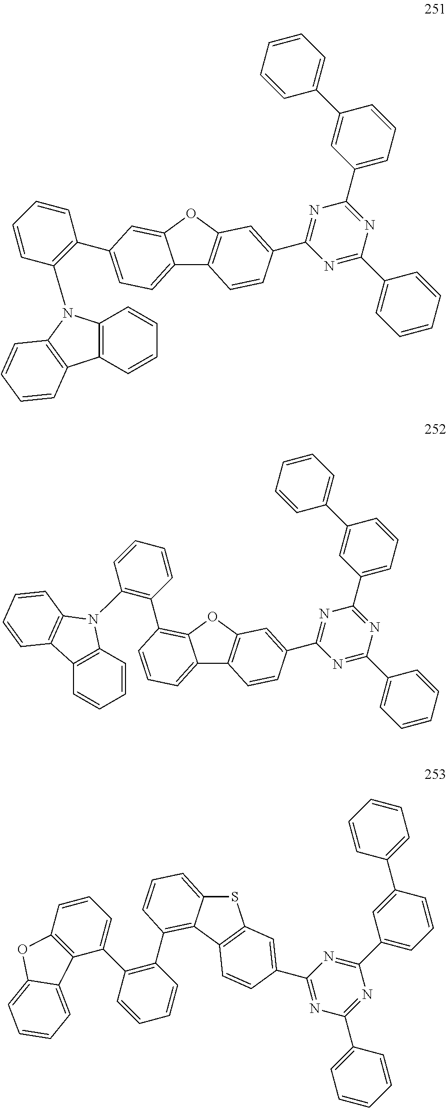 [SMILES: c1ccc(-c2cccc(-c3nc(-c4ccccc4)nc(-c4ccc5c(c4)oc4cc(-c6ccccc6-n6c7ccccc7c7ccccc76)ccc45)n3)c2)cc1 ; c1ccc(-c2cccc(-c3nc(-c4ccccc4)nc(-c4ccc5c(c4)oc4c(-c6ccccc6-n6c7ccccc7c7ccccc76)cccc45)n3)c2)cc1 ; c1ccc(-c2cccc(-c3nc(-c4ccccc4)nc(-c4ccc5c(c4)sc4cccc(-c6ccccc6-c6cccc7oc8ccccc8c67)c45)n3)c2)cc1]

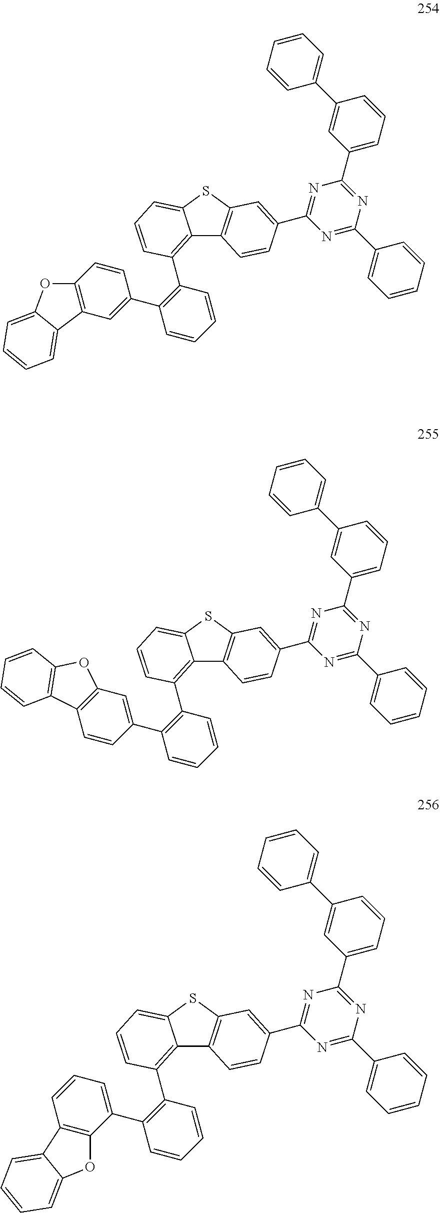 [SMILES: c1ccc(-c2cccc(-c3nc(-c4ccccc4)nc(-c4ccc5c(c4)sc4cccc(-c6ccccc6-c6ccc7oc8ccccc8c7c6)c45)n3)c2)cc1 ; c1ccc(-c2cccc(-c3nc(-c4ccccc4)nc(-c4ccc5c(c4)sc4cccc(-c6ccccc6-c6ccc7c(c6)oc6ccccc67)c45)n3)c2)cc1 ; c1ccc(-c2cccc(-c3nc(-c4ccccc4)nc(-c4ccc5c(c4)sc4cccc(-c6ccccc6-c6cccc7c6oc6ccccc67)c45)n3)c2)cc1]

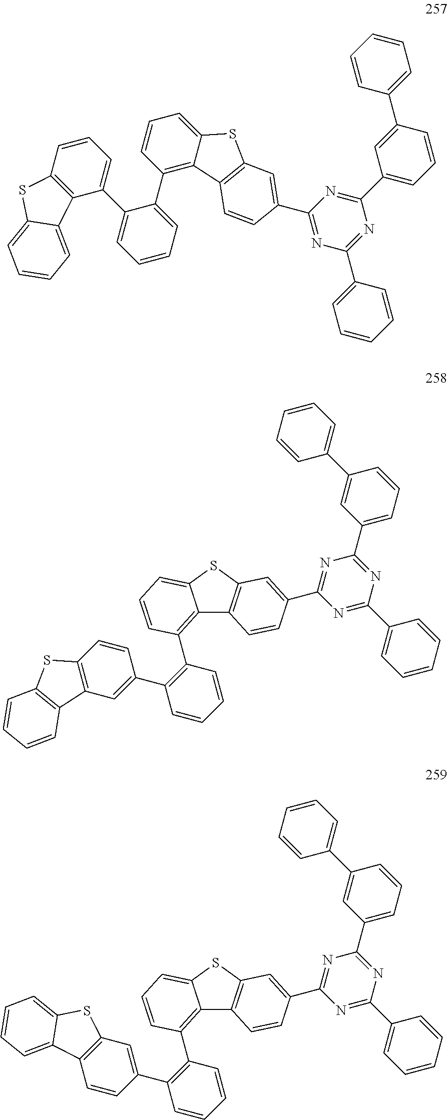 [SMILES: c1ccc(-c2cccc(-c3nc(-c4ccccc4)nc(-c4ccc5c(c4)sc4cccc(-c6ccccc6-c6cccc7sc8ccccc8c67)c45)n3)c2)cc1 ; c1ccc(-c2cccc(-c3nc(-c4ccccc4)nc(-c4ccc5c(c4)sc4cccc(-c6ccccc6-c6ccc7sc8ccccc8c7c6)c45)n3)c2)cc1 ; c1ccc(-c2cccc(-c3nc(-c4ccccc4)nc(-c4ccc5c(c4)sc4cccc(-c6ccccc6-c6ccc7c(c6)sc6ccccc67)c45)n3)c2)cc1]

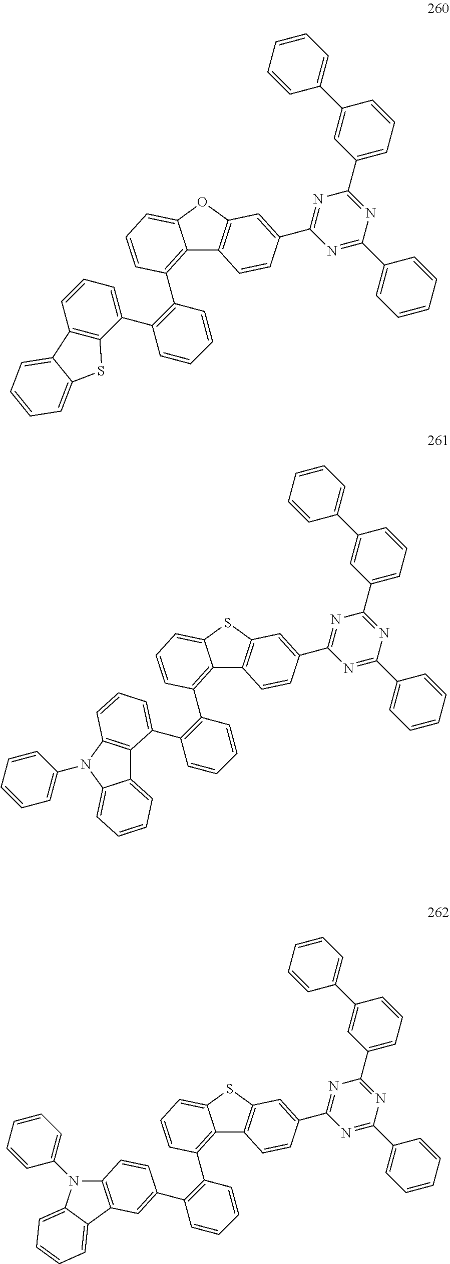 [SMILES: c1ccc(-c2cccc(-c3nc(-c4ccccc4)nc(-c4ccc5c(c4)oc4cccc(-c6ccccc6-c6cccc7c6sc6ccccc67)c45)n3)c2)cc1 ; c1ccc(-c2cccc(-c3nc(-c4ccccc4)nc(-c4ccc5c(c4)sc4cccc(-c6ccccc6-c6cccc7c6c6ccccc6n7-c6ccccc6)c45)n3)c2)cc1 ; c1ccc(-c2cccc(-c3nc(-c4ccccc4)nc(-c4ccc5c(c4)sc4cccc(-c6ccccc6-c6ccc7c(c6)c6ccccc6n7-c6ccccc6)c45)n3)c2)cc1]

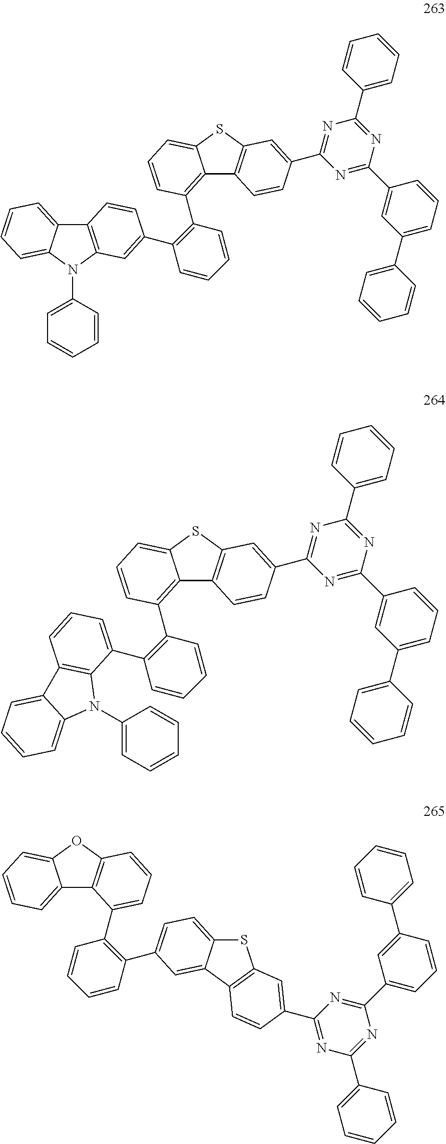 [SMILES: c1ccc(-c2cccc(-c3nc(-c4ccccc4)nc(-c4ccc5c(c4)sc4cccc(-c6ccccc6-c6ccc7c8ccccc8n(-c8ccccc8)c7c6)c45)n3)c2)cc1 ; c1ccc(-c2cccc(-c3nc(-c4ccccc4)nc(-c4ccc5c(c4)sc4cccc(-c6ccccc6-c6cccc7c8ccccc8n(-c8ccccc8)c67)c45)n3)c2)cc1 ; c1ccc(-c2cccc(-c3nc(-c4ccccc4)nc(-c4ccc5c(c4)sc4ccc(-c6ccccc6-c6cccc7oc8ccccc8c67)cc45)n3)c2)cc1]

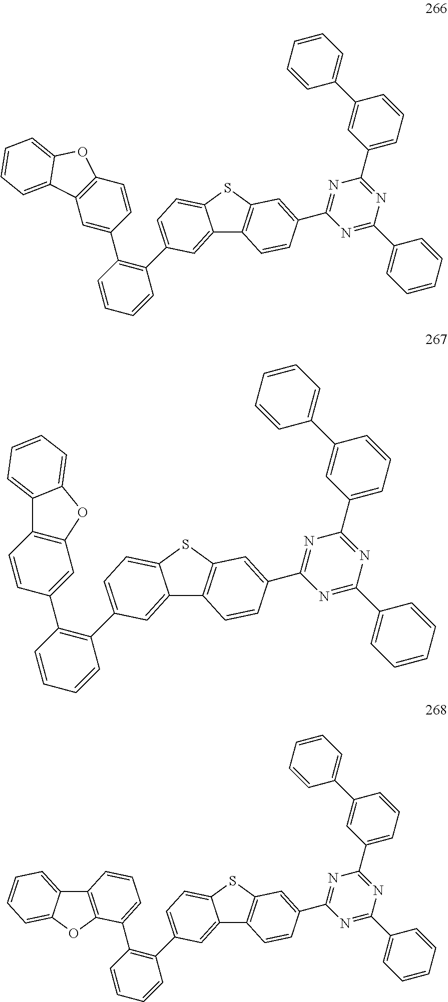 [SMILES: c1ccc(-c2cccc(-c3nc(-c4ccccc4)nc(-c4ccc5c(c4)sc4ccc(-c6ccccc6-c6ccc7oc8ccccc8c7c6)cc45)n3)c2)cc1 ; c1ccc(-c2cccc(-c3nc(-c4ccccc4)nc(-c4ccc5c(c4)sc4ccc(-c6ccccc6-c6ccc7c(c6)oc6ccccc67)cc45)n3)c2)cc1 ; c1ccc(-c2cccc(-c3nc(-c4ccccc4)nc(-c4ccc5c(c4)sc4ccc(-c6ccccc6-c6cccc7c6oc6ccccc67)cc45)n3)c2)cc1]

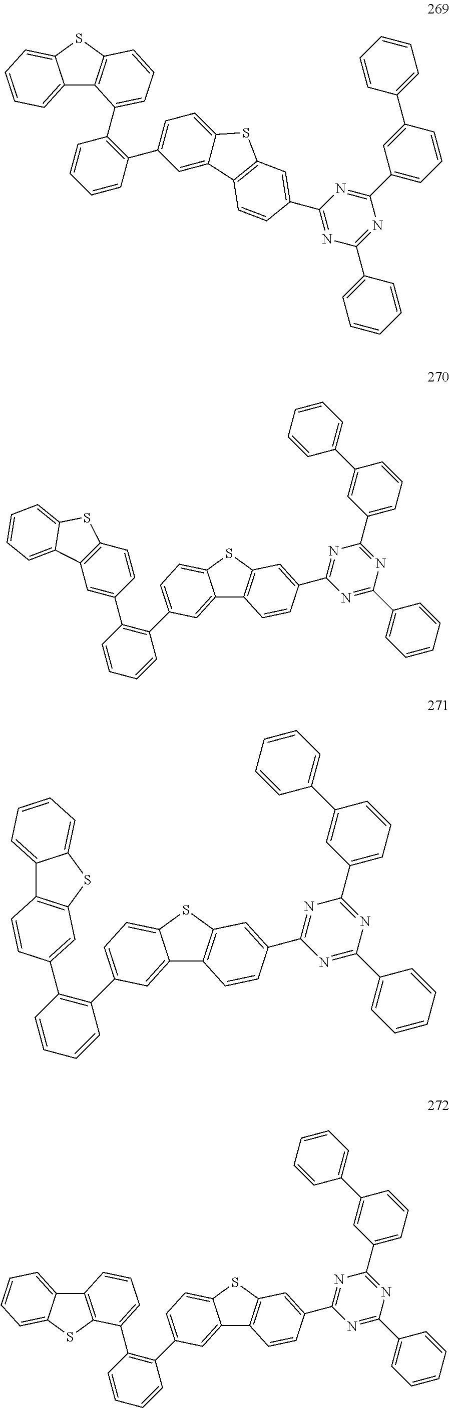 [SMILES: c1ccc(-c2cccc(-c3nc(-c4ccccc4)nc(-c4ccc5c(c4)sc4ccc(-c6ccccc6-c6cccc7sc8ccccc8c67)cc45)n3)c2)cc1 ; c1ccc(-c2cccc(-c3nc(-c4ccccc4)nc(-c4ccc5c(c4)sc4ccc(-c6ccccc6-c6ccc7sc8ccccc8c7c6)cc45)n3)c2)cc1 ; c1ccc(-c2cccc(-c3nc(-c4ccccc4)nc(-c4ccc5c(c4)sc4ccc(-c6ccccc6-c6ccc7c(c6)sc6ccccc67)cc45)n3)c2)cc1 ; c1ccc(-c2cccc(-c3nc(-c4ccccc4)nc(-c4ccc5c(c4)sc4ccc(-c6ccccc6-c6cccc7c6sc6ccccc67)cc45)n3)c2)cc1]

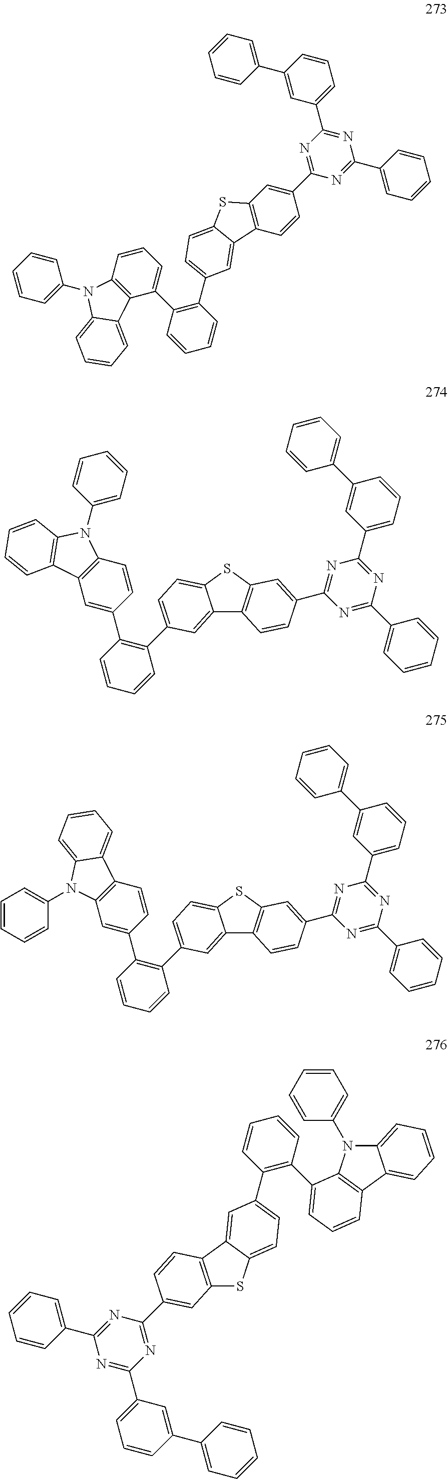 [SMILES: c1ccc(-c2cccc(-c3nc(-c4ccccc4)nc(-c4ccc5c(c4)sc4ccc(-c6ccccc6-c6cccc7c6c6ccccc6n7-c6ccccc6)cc45)n3)c2)cc1 ; c1ccc(-c2cccc(-c3nc(-c4ccccc4)nc(-c4ccc5c(c4)sc4ccc(-c6ccccc6-c6ccc7c(c6)c6ccccc6n7-c6ccccc6)cc45)n3)c2)cc1 ; c1ccc(-c2cccc(-c3nc(-c4ccccc4)nc(-c4ccc5c(c4)sc4ccc(-c6ccccc6-c6ccc7c8ccccc8n(-c8ccccc8)c7c6)cc45)n3)c2)cc1 ; c1ccc(-c2cccc(-c3nc(-c4ccccc4)nc(-c4ccc5c(c4)sc4ccc(-c6ccccc6-c6cccc7c8ccccc8n(-c8ccccc8)c67)cc45)n3)c2)cc1]

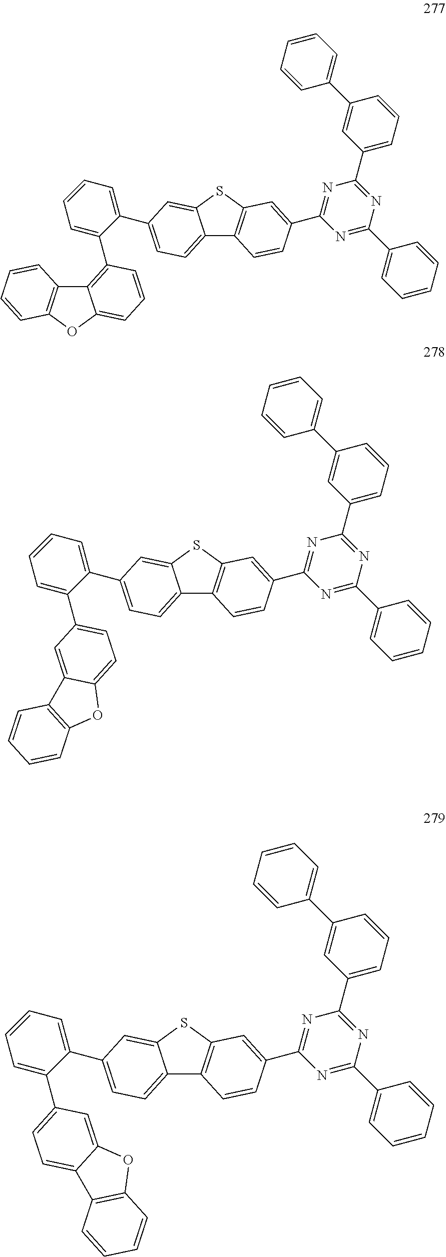 [SMILES: c1ccc(-c2cccc(-c3nc(-c4ccccc4)nc(-c4ccc5c(c4)sc4cc(-c6ccccc6-c6cccc7oc8ccccc8c67)ccc45)n3)c2)cc1 ; c1ccc(-c2cccc(-c3nc(-c4ccccc4)nc(-c4ccc5c(c4)sc4cc(-c6ccccc6-c6ccc7oc8ccccc8c7c6)ccc45)n3)c2)cc1 ; c1ccc(-c2cccc(-c3nc(-c4ccccc4)nc(-c4ccc5c(c4)sc4cc(-c6ccccc6-c6ccc7c(c6)oc6ccccc67)ccc45)n3)c2)cc1]

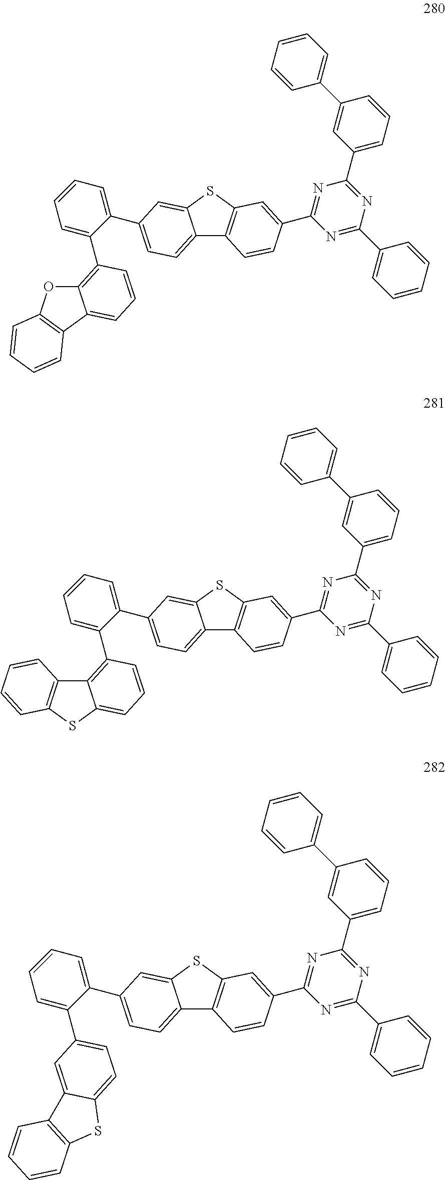 [SMILES: c1ccc(-c2cccc(-c3nc(-c4ccccc4)nc(-c4ccc5c(c4)sc4cc(-c6ccccc6-c6cccc7c6oc6ccccc67)ccc45)n3)c2)cc1 ; c1ccc(-c2cccc(-c3nc(-c4ccccc4)nc(-c4ccc5c(c4)sc4cc(-c6ccccc6-c6cccc7sc8ccccc8c67)ccc45)n3)c2)cc1 ; c1ccc(-c2cccc(-c3nc(-c4ccccc4)nc(-c4ccc5c(c4)sc4cc(-c6ccccc6-c6ccc7sc8ccccc8c7c6)ccc45)n3)c2)cc1]

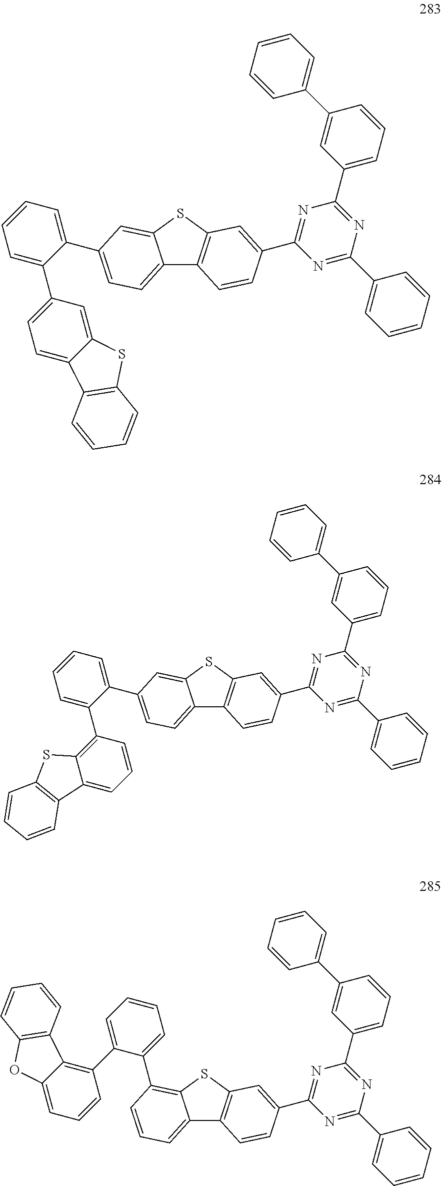 [SMILES: c1ccc(-c2cccc(-c3nc(-c4ccccc4)nc(-c4ccc5c(c4)sc4cc(-c6ccccc6-c6ccc7c(c6)sc6ccccc67)ccc45)n3)c2)cc1 ; c1ccc(-c2cccc(-c3nc(-c4ccccc4)nc(-c4ccc5c(c4)sc4cc(-c6ccccc6-c6cccc7c6sc6ccccc67)ccc45)n3)c2)cc1 ; c1ccc(-c2cccc(-c3nc(-c4ccccc4)nc(-c4ccc5c(c4)sc4c(-c6ccccc6-c6cccc7oc8ccccc8c67)cccc45)n3)c2)cc1]

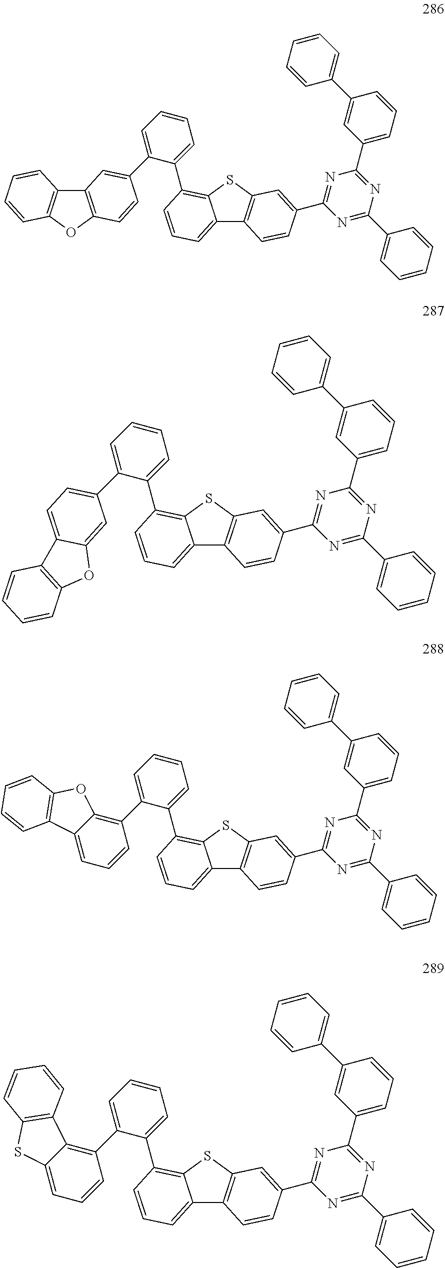 [SMILES: c1ccc(-c2cccc(-c3nc(-c4ccccc4)nc(-c4ccc5c(c4)sc4c(-c6ccccc6-c6ccc7oc8ccccc8c7c6)cccc45)n3)c2)cc1 ; c1ccc(-c2cccc(-c3nc(-c4ccccc4)nc(-c4ccc5c(c4)sc4c(-c6ccccc6-c6ccc7c(c6)oc6ccccc67)cccc45)n3)c2)cc1 ; c1ccc(-c2cccc(-c3nc(-c4ccccc4)nc(-c4ccc5c(c4)sc4c(-c6ccccc6-c6cccc7c6oc6ccccc67)cccc45)n3)c2)cc1 ; c1ccc(-c2cccc(-c3nc(-c4ccccc4)nc(-c4ccc5c(c4)sc4c(-c6ccccc6-c6cccc7sc8ccccc8c67)cccc45)n3)c2)cc1]

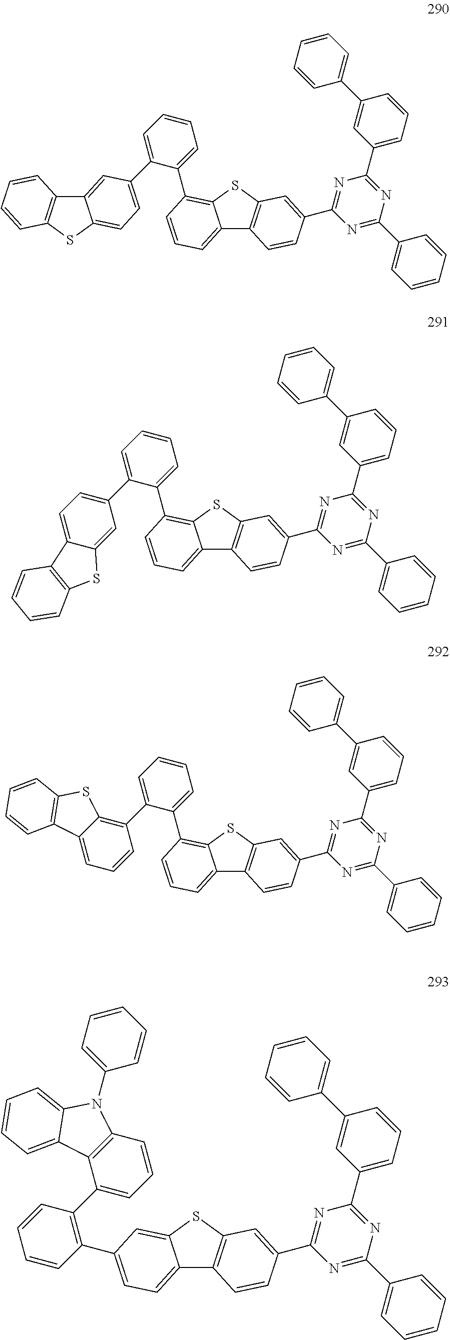 [SMILES: c1ccc(-c2cccc(-c3nc(-c4ccccc4)nc(-c4ccc5c(c4)sc4c(-c6ccccc6-c6ccc7sc8ccccc8c7c6)cccc45)n3)c2)cc1 ; c1ccc(-c2cccc(-c3nc(-c4ccccc4)nc(-c4ccc5c(c4)sc4c(-c6ccccc6-c6ccc7c(c6)sc6ccccc67)cccc45)n3)c2)cc1 ; c1ccc(-c2cccc(-c3nc(-c4ccccc4)nc(-c4ccc5c(c4)sc4c(-c6ccccc6-c6cccc7c6sc6ccccc67)cccc45)n3)c2)cc1 ; c1ccc(-c2cccc(-c3nc(-c4ccccc4)nc(-c4ccc5c(c4)sc4cc(-c6ccccc6-c6cccc7c6c6ccccc6n7-c6ccccc6)ccc45)n3)c2)cc1]

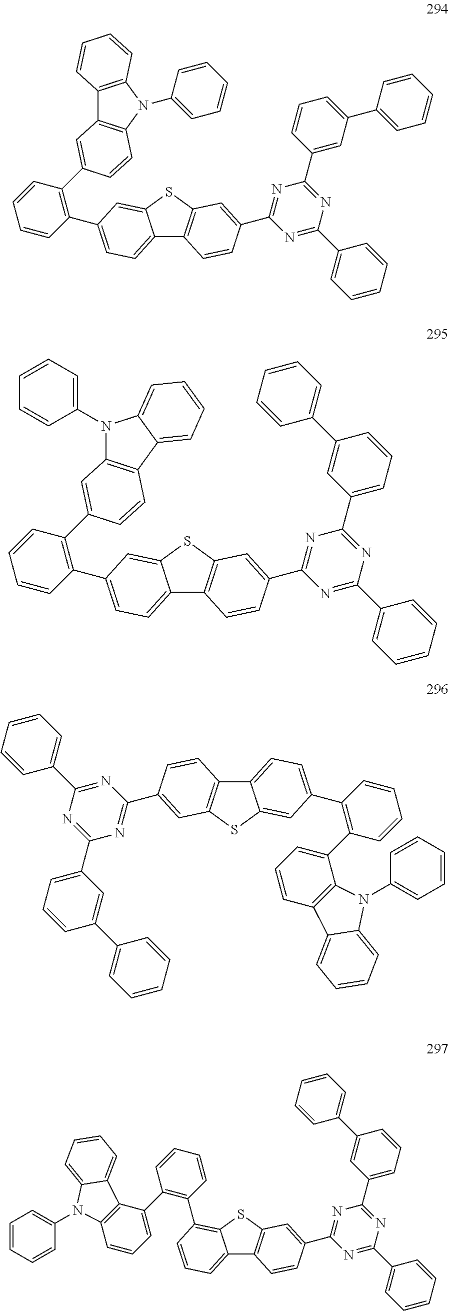 [SMILES: c1ccc(-c2cccc(-c3nc(-c4ccccc4)nc(-c4ccc5c(c4)sc4cc(-c6ccccc6-c6ccc7c(c6)c6ccccc6n7-c6ccccc6)ccc45)n3)c2)cc1 ; c1ccc(-c2cccc(-c3nc(-c4ccccc4)nc(-c4ccc5c(c4)sc4cc(-c6ccccc6-c6ccc7c8ccccc8n(-c8ccccc8)c7c6)ccc45)n3)c2)cc1 ; c1ccc(-c2cccc(-c3nc(-c4ccccc4)nc(-c4ccc5c(c4)sc4cc(-c6ccccc6-c6cccc7c8ccccc8n(-c8ccccc8)c67)ccc45)n3)c2)cc1 ; c1ccc(-c2cccc(-c3nc(-c4ccccc4)nc(-c4ccc5c(c4)sc4c(-c6ccccc6-c6cccc7c6c6ccccc6n7-c6ccccc6)cccc45)n3)c2)cc1]

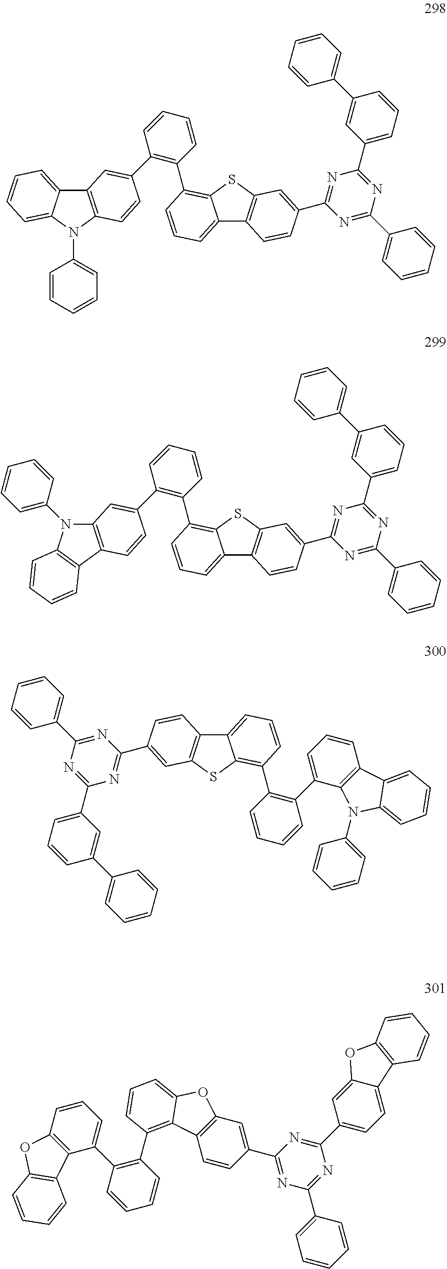 [SMILES: c1ccc(-c2cccc(-c3nc(-c4ccccc4)nc(-c4ccc5c(c4)sc4c(-c6ccccc6-c6ccc7c(c6)c6ccccc6n7-c6ccccc6)cccc45)n3)c2)cc1 ; c1ccc(-c2cccc(-c3nc(-c4ccccc4)nc(-c4ccc5c(c4)sc4c(-c6ccccc6-c6ccc7c8ccccc8n(-c8ccccc8)c7c6)cccc45)n3)c2)cc1 ; c1ccc(-c2cccc(-c3nc(-c4ccccc4)nc(-c4ccc5c(c4)sc4c(-c6ccccc6-c6cccc7c8ccccc8n(-c8ccccc8)c67)cccc45)n3)c2)cc1 ; c1ccc(-c2nc(-c3ccc4c(c3)oc3ccccc34)nc(-c3ccc4c(c3)oc3cccc(-c5ccccc5-c5cccc6oc7ccccc7c56)c34)n2)cc1]

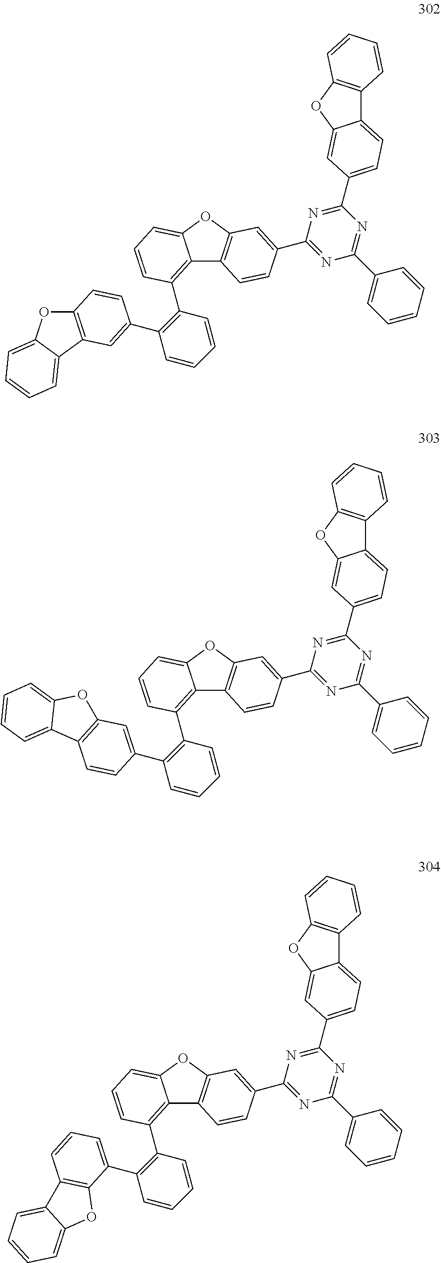 [SMILES: c1ccc(-c2nc(-c3ccc4c(c3)oc3ccccc34)nc(-c3ccc4c(c3)oc3cccc(-c5ccccc5-c5ccc6oc7ccccc7c6c5)c34)n2)cc1 ; c1ccc(-c2nc(-c3ccc4c(c3)oc3ccccc34)nc(-c3ccc4c(c3)oc3cccc(-c5ccccc5-c5ccc6c(c5)oc5ccccc56)c34)n2)cc1 ; c1ccc(-c2nc(-c3ccc4c(c3)oc3ccccc34)nc(-c3ccc4c(c3)oc3cccc(-c5ccccc5-c5cccc6c5oc5ccccc56)c34)n2)cc1]

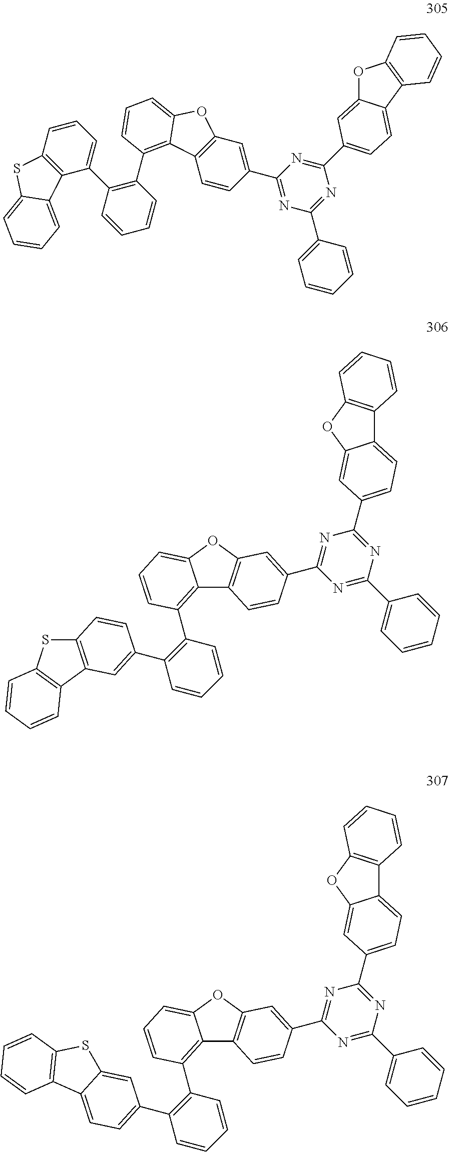 [SMILES: c1ccc(-c2nc(-c3ccc4c(c3)oc3ccccc34)nc(-c3ccc4c(c3)oc3cccc(-c5ccccc5-c5cccc6sc7ccccc7c56)c34)n2)cc1 ; c1ccc(-c2nc(-c3ccc4c(c3)oc3ccccc34)nc(-c3ccc4c(c3)oc3cccc(-c5ccccc5-c5ccc6sc7ccccc7c6c5)c34)n2)cc1 ; c1ccc(-c2nc(-c3ccc4c(c3)oc3ccccc34)nc(-c3ccc4c(c3)oc3cccc(-c5ccccc5-c5ccc6c(c5)sc5ccccc56)c34)n2)cc1]

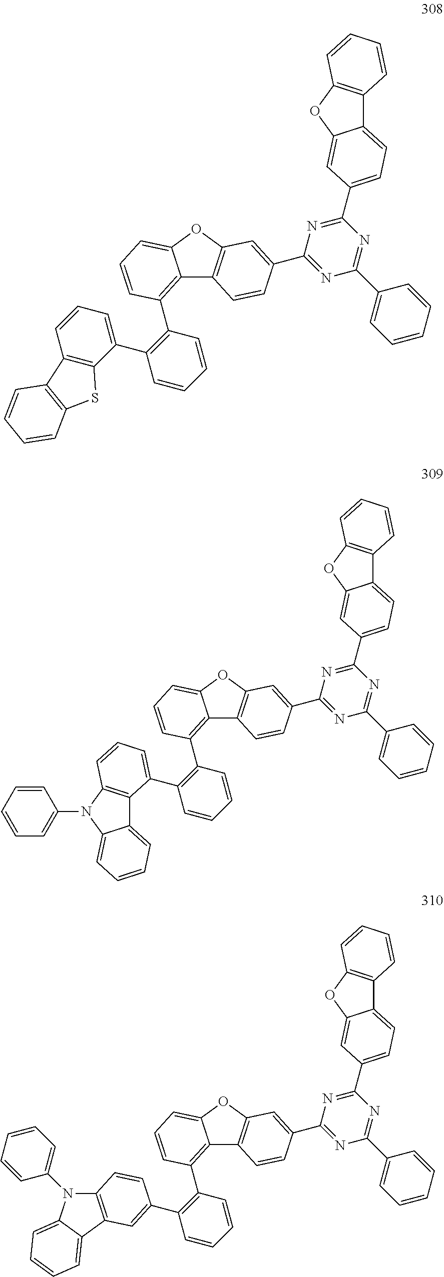 [SMILES: c1ccc(-c2nc(-c3ccc4c(c3)oc3ccccc34)nc(-c3ccc4c(c3)oc3cccc(-c5ccccc5-c5cccc6c5sc5ccccc56)c34)n2)cc1 ; c1ccc(-c2nc(-c3ccc4c(c3)oc3ccccc34)nc(-c3ccc4c(c3)oc3cccc(-c5ccccc5-c5cccc6c5c5ccccc5n6-c5ccccc5)c34)n2)cc1 ; c1ccc(-c2nc(-c3ccc4c(c3)oc3ccccc34)nc(-c3ccc4c(c3)oc3cccc(-c5ccccc5-c5ccc6c(c5)c5ccccc5n6-c5ccccc5)c34)n2)cc1]

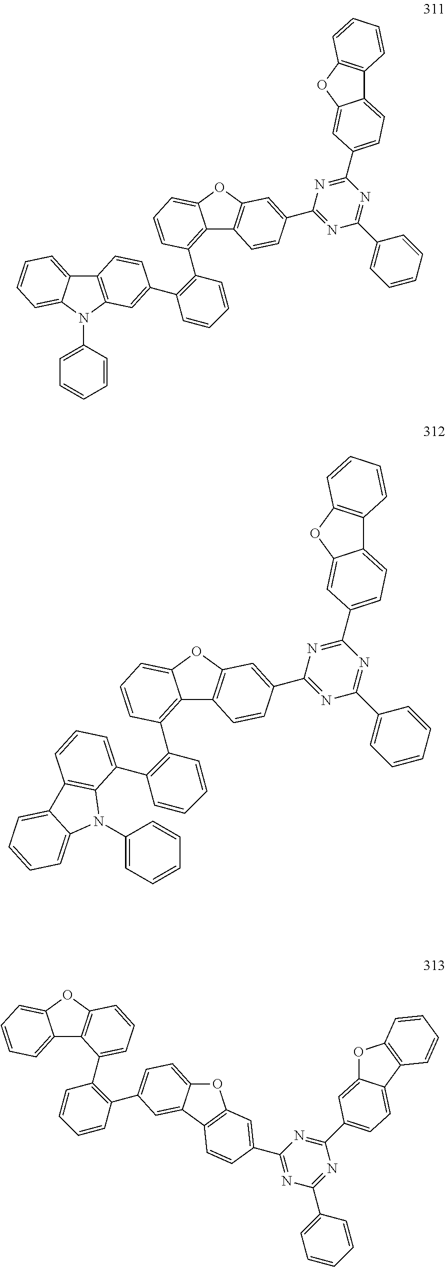 [SMILES: c1ccc(-c2nc(-c3ccc4c(c3)oc3ccccc34)nc(-c3ccc4c(c3)oc3cccc(-c5ccccc5-c5ccc6c7ccccc7n(-c7ccccc7)c6c5)c34)n2)cc1 ; c1ccc(-c2nc(-c3ccc4c(c3)oc3ccccc34)nc(-c3ccc4c(c3)oc3cccc(-c5ccccc5-c5cccc6c7ccccc7n(-c7ccccc7)c56)c34)n2)cc1 ; c1ccc(-c2nc(-c3ccc4c(c3)oc3ccccc34)nc(-c3ccc4c(c3)oc3ccc(-c5ccccc5-c5cccc6oc7ccccc7c56)cc34)n2)cc1]

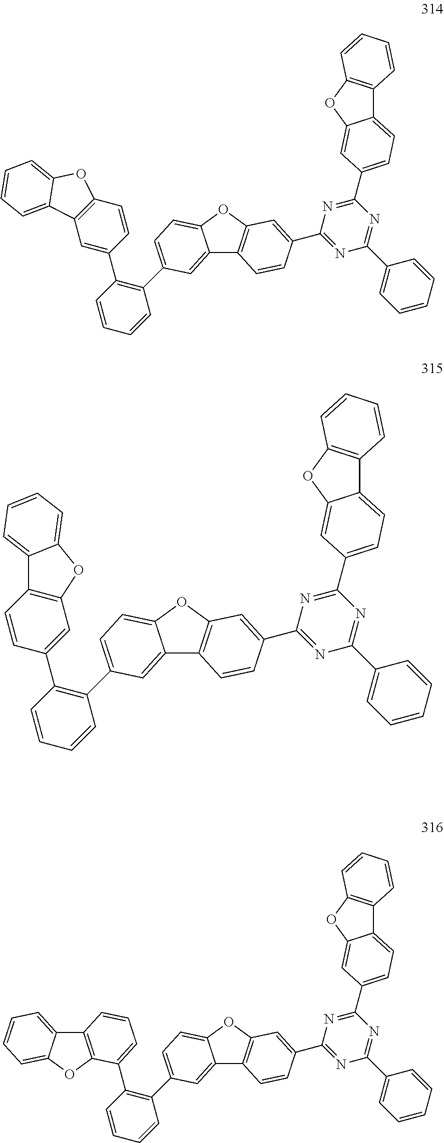 [SMILES: c1ccc(-c2nc(-c3ccc4c(c3)oc3ccccc34)nc(-c3ccc4c(c3)oc3ccc(-c5ccccc5-c5ccc6oc7ccccc7c6c5)cc34)n2)cc1 ; c1ccc(-c2nc(-c3ccc4c(c3)oc3ccccc34)nc(-c3ccc4c(c3)oc3ccc(-c5ccccc5-c5ccc6c(c5)oc5ccccc56)cc34)n2)cc1 ; c1ccc(-c2nc(-c3ccc4c(c3)oc3ccccc34)nc(-c3ccc4c(c3)oc3ccc(-c5ccccc5-c5cccc6c5oc5ccccc56)cc34)n2)cc1]

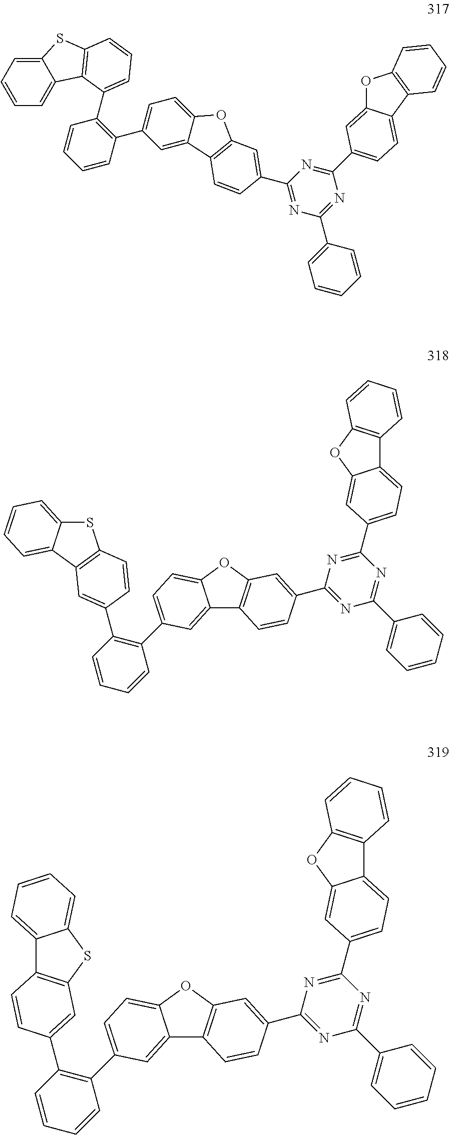 [SMILES: c1ccc(-c2nc(-c3ccc4c(c3)oc3ccccc34)nc(-c3ccc4c(c3)oc3ccc(-c5ccccc5-c5cccc6sc7ccccc7c56)cc34)n2)cc1 ; c1ccc(-c2nc(-c3ccc4c(c3)oc3ccccc34)nc(-c3ccc4c(c3)oc3ccc(-c5ccccc5-c5ccc6sc7ccccc7c6c5)cc34)n2)cc1 ; c1ccc(-c2nc(-c3ccc4c(c3)oc3ccccc34)nc(-c3ccc4c(c3)oc3ccc(-c5ccccc5-c5ccc6c(c5)sc5ccccc56)cc34)n2)cc1]

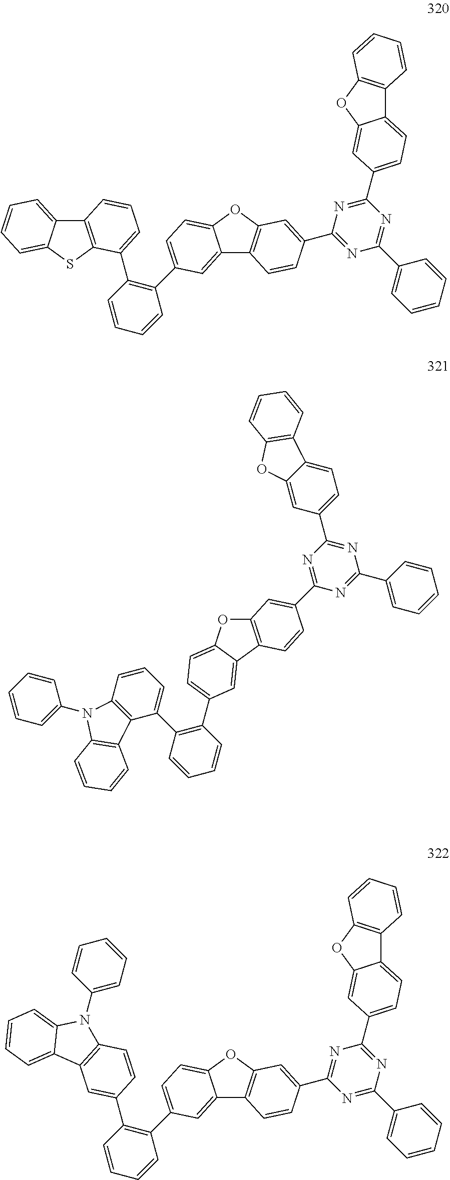 [SMILES: c1ccc(-c2nc(-c3ccc4c(c3)oc3ccccc34)nc(-c3ccc4c(c3)oc3ccc(-c5ccccc5-c5cccc6c5sc5ccccc56)cc34)n2)cc1 ; c1ccc(-c2nc(-c3ccc4c(c3)oc3ccccc34)nc(-c3ccc4c(c3)oc3ccc(-c5ccccc5-c5cccc6c5c5ccccc5n6-c5ccccc5)cc34)n2)cc1 ; c1ccc(-c2nc(-c3ccc4c(c3)oc3ccccc34)nc(-c3ccc4c(c3)oc3ccc(-c5ccccc5-c5ccc6c(c5)c5ccccc5n6-c5ccccc5)cc34)n2)cc1]

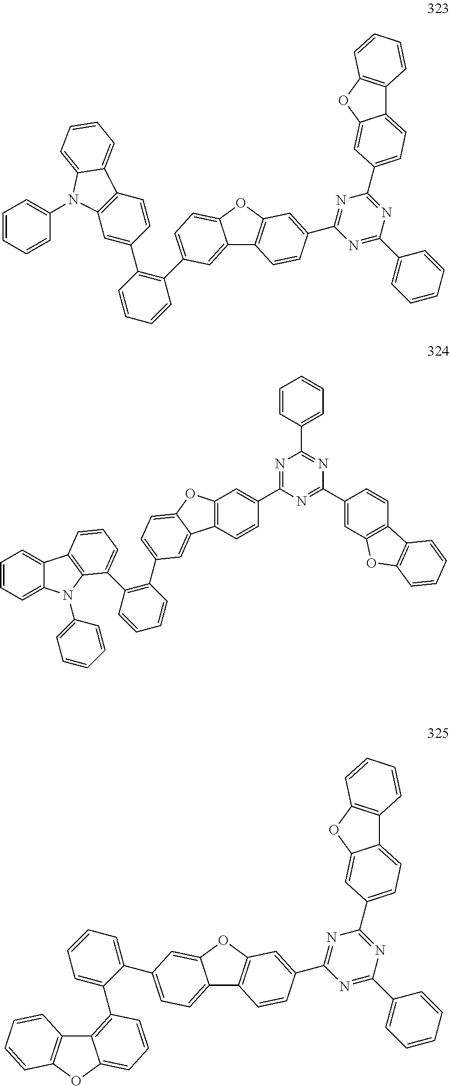 [SMILES: c1ccc(-c2nc(-c3ccc4c(c3)oc3ccccc34)nc(-c3ccc4c(c3)oc3ccc(-c5ccccc5-c5ccc6c7ccccc7n(-c7ccccc7)c6c5)cc34)n2)cc1 ; c1ccc(-c2nc(-c3ccc4c(c3)oc3ccccc34)nc(-c3ccc4c(c3)oc3ccc(-c5ccccc5-c5cccc6c7ccccc7n(-c7ccccc7)c56)cc34)n2)cc1 ; c1ccc(-c2nc(-c3ccc4c(c3)oc3ccccc34)nc(-c3ccc4c(c3)oc3cc(-c5ccccc5-c5cccc6oc7ccccc7c56)ccc34)n2)cc1]

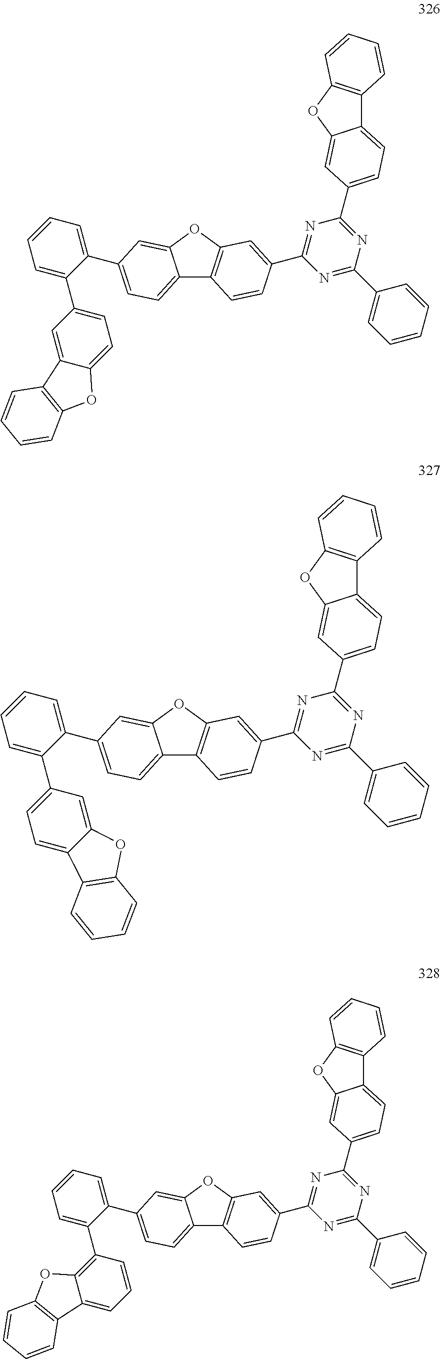 [SMILES: c1ccc(-c2nc(-c3ccc4c(c3)oc3ccccc34)nc(-c3ccc4c(c3)oc3cc(-c5ccccc5-c5ccc6oc7ccccc7c6c5)ccc34)n2)cc1 ; c1ccc(-c2nc(-c3ccc4c(c3)oc3ccccc34)nc(-c3ccc4c(c3)oc3cc(-c5ccccc5-c5ccc6c(c5)oc5ccccc56)ccc34)n2)cc1 ; c1ccc(-c2nc(-c3ccc4c(c3)oc3ccccc34)nc(-c3ccc4c(c3)oc3cc(-c5ccccc5-c5cccc6c5oc5ccccc56)ccc34)n2)cc1]

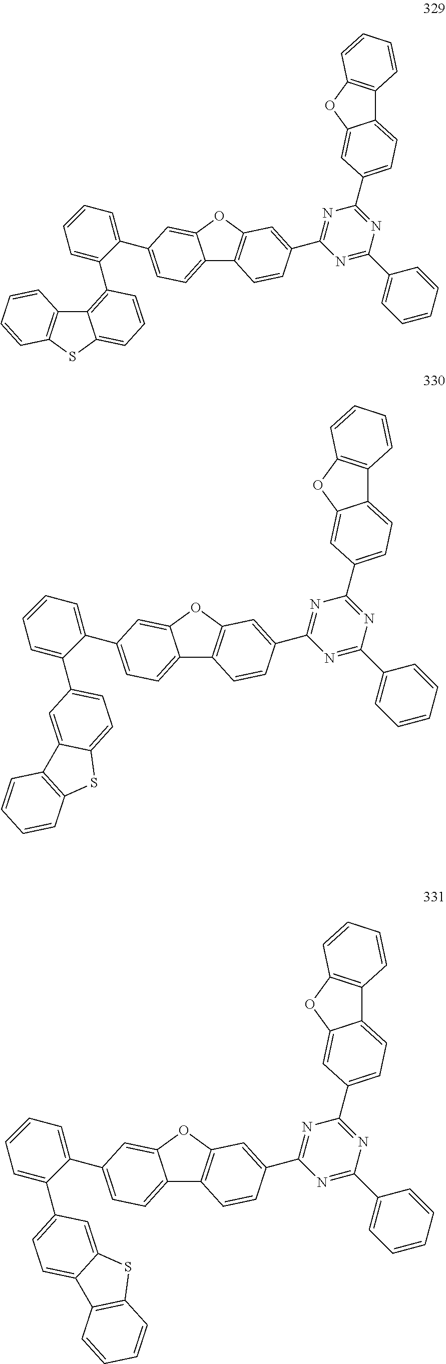 [SMILES: c1ccc(-c2nc(-c3ccc4c(c3)oc3ccccc34)nc(-c3ccc4c(c3)oc3cc(-c5ccccc5-c5cccc6sc7ccccc7c56)ccc34)n2)cc1 ; c1ccc(-c2nc(-c3ccc4c(c3)oc3ccccc34)nc(-c3ccc4c(c3)oc3cc(-c5ccccc5-c5ccc6sc7ccccc7c6c5)ccc34)n2)cc1 ; c1ccc(-c2nc(-c3ccc4c(c3)oc3ccccc34)nc(-c3ccc4c(c3)oc3cc(-c5ccccc5-c5ccc6c(c5)sc5ccccc56)ccc34)n2)cc1]

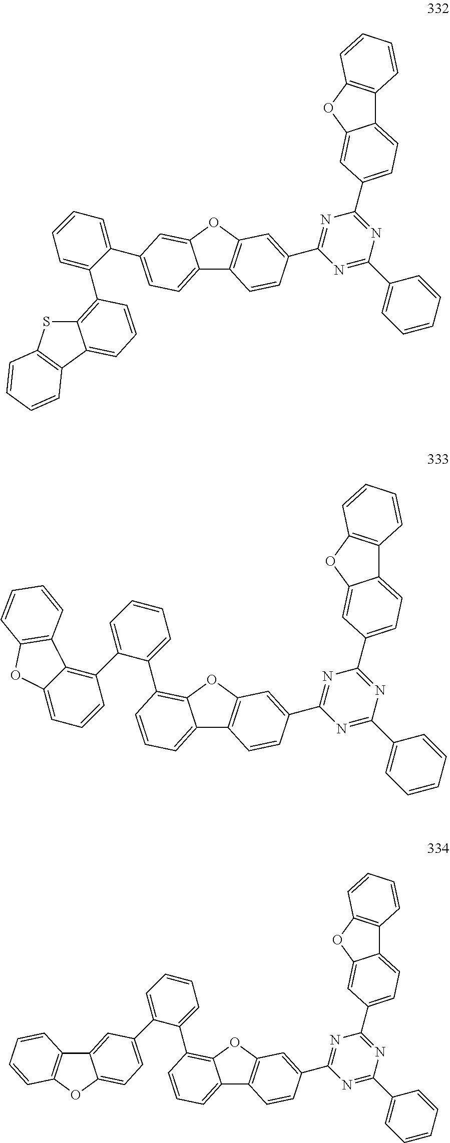 [SMILES: c1ccc(-c2nc(-c3ccc4c(c3)oc3ccccc34)nc(-c3ccc4c(c3)oc3cc(-c5ccccc5-c5cccc6c5sc5ccccc56)ccc34)n2)cc1 ; c1ccc(-c2nc(-c3ccc4c(c3)oc3ccccc34)nc(-c3ccc4c(c3)oc3c(-c5ccccc5-c5cccc6oc7ccccc7c56)cccc34)n2)cc1 ; c1ccc(-c2nc(-c3ccc4c(c3)oc3ccccc34)nc(-c3ccc4c(c3)oc3c(-c5ccccc5-c5ccc6oc7ccccc7c6c5)cccc34)n2)cc1]

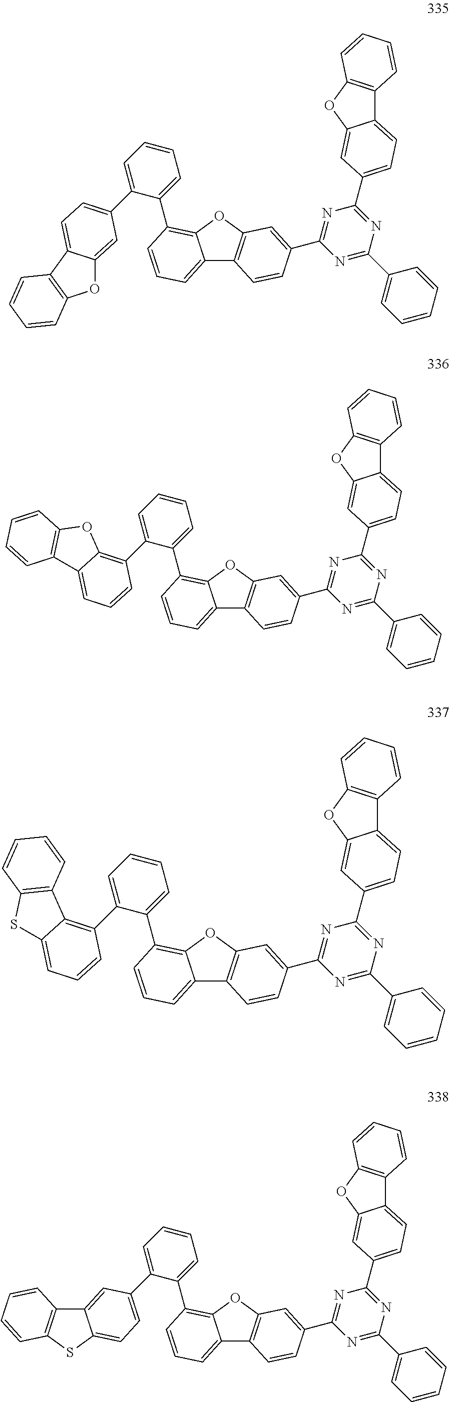 [SMILES: c1ccc(-c2nc(-c3ccc4c(c3)oc3ccccc34)nc(-c3ccc4c(c3)oc3c(-c5ccccc5-c5ccc6c(c5)oc5ccccc56)cccc34)n2)cc1 ; c1ccc(-c2nc(-c3ccc4c(c3)oc3ccccc34)nc(-c3ccc4c(c3)oc3c(-c5ccccc5-c5cccc6c5oc5ccccc56)cccc34)n2)cc1 ; c1ccc(-c2nc(-c3ccc4c(c3)oc3ccccc34)nc(-c3ccc4c(c3)oc3c(-c5ccccc5-c5cccc6sc7ccccc7c56)cccc34)n2)cc1 ; c1ccc(-c2nc(-c3ccc4c(c3)oc3ccccc34)nc(-c3ccc4c(c3)oc3c(-c5ccccc5-c5ccc6sc7ccccc7c6c5)cccc34)n2)cc1]

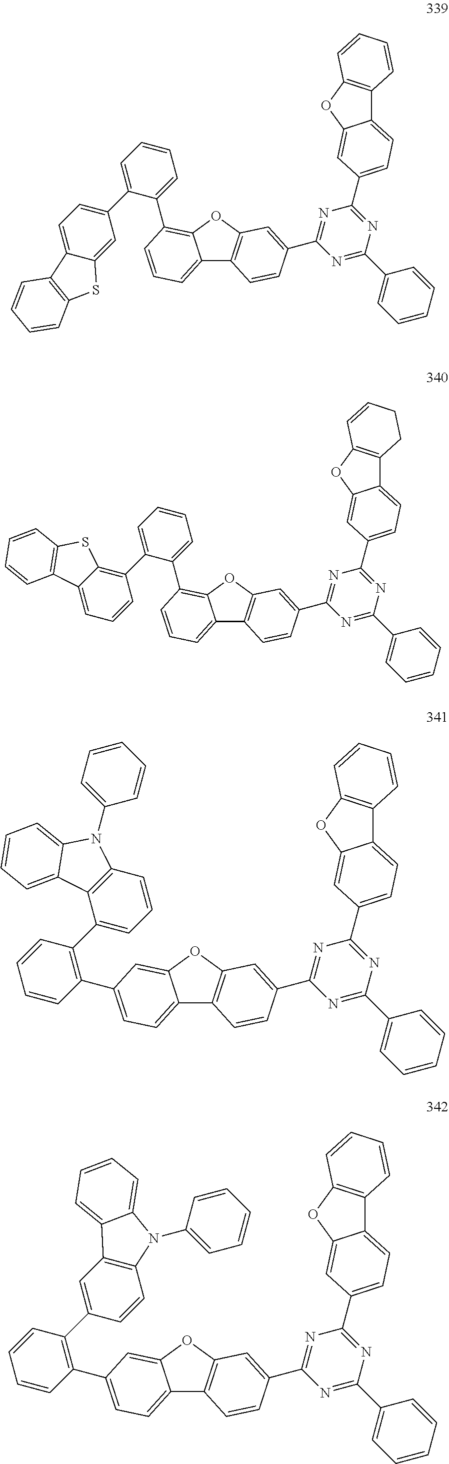 [SMILES: c1ccc(-c2nc(-c3ccc4c(c3)oc3ccccc34)nc(-c3ccc4c(c3)oc3c(-c5ccccc5-c5ccc6c(c5)sc5ccccc56)cccc34)n2)cc1 ; C1=Cc2oc3cc(-c4nc(-c5ccccc5)nc(-c5ccc6c(c5)oc5c(-c7ccccc7-c7cccc8c7sc7ccccc78)cccc56)n4)ccc3c2CC1 ; c1ccc(-c2nc(-c3ccc4c(c3)oc3ccccc34)nc(-c3ccc4c(c3)oc3cc(-c5ccccc5-c5cccc6c5c5ccccc5n6-c5ccccc5)ccc34)n2)cc1 ; c1ccc(-c2nc(-c3ccc4c(c3)oc3ccccc34)nc(-c3ccc4c(c3)oc3cc(-c5ccccc5-c5ccc6c(c5)c5ccccc5n6-c5ccccc5)ccc34)n2)cc1]

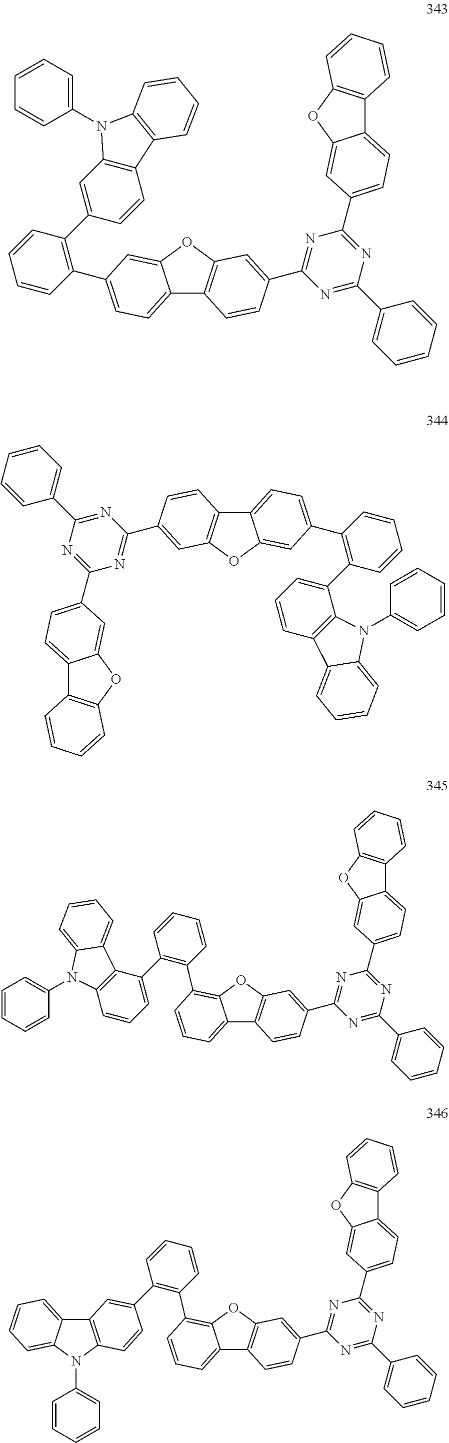 [SMILES: c1ccc(-c2nc(-c3ccc4c(c3)oc3ccccc34)nc(-c3ccc4c(c3)oc3cc(-c5ccccc5-c5ccc6c7ccccc7n(-c7ccccc7)c6c5)ccc34)n2)cc1 ; c1ccc(-c2nc(-c3ccc4c(c3)oc3ccccc34)nc(-c3ccc4c(c3)oc3cc(-c5ccccc5-c5cccc6c7ccccc7n(-c7ccccc7)c56)ccc34)n2)cc1 ; c1ccc(-c2nc(-c3ccc4c(c3)oc3ccccc34)nc(-c3ccc4c(c3)oc3c(-c5ccccc5-c5cccc6c5c5ccccc5n6-c5ccccc5)cccc34)n2)cc1 ; c1ccc(-c2nc(-c3ccc4c(c3)oc3ccccc34)nc(-c3ccc4c(c3)oc3c(-c5ccccc5-c5ccc6c(c5)c5ccccc5n6-c5ccccc5)cccc34)n2)cc1]

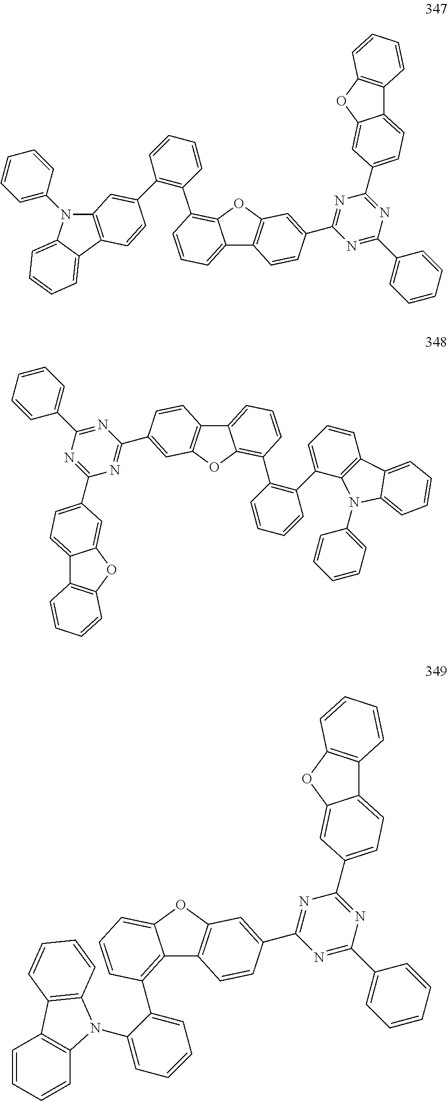 [SMILES: c1ccc(-c2nc(-c3ccc4c(c3)oc3ccccc34)nc(-c3ccc4c(c3)oc3c(-c5ccccc5-c5ccc6c7ccccc7n(-c7ccccc7)c6c5)cccc34)n2)cc1 ; c1ccc(-c2nc(-c3ccc4c(c3)oc3ccccc34)nc(-c3ccc4c(c3)oc3c(-c5ccccc5-c5cccc6c7ccccc7n(-c7ccccc7)c56)cccc34)n2)cc1 ; c1ccc(-c2nc(-c3ccc4c(c3)oc3ccccc34)nc(-c3ccc4c(c3)oc3cccc(-c5ccccc5-n5c6ccccc6c6ccccc65)c34)n2)cc1]

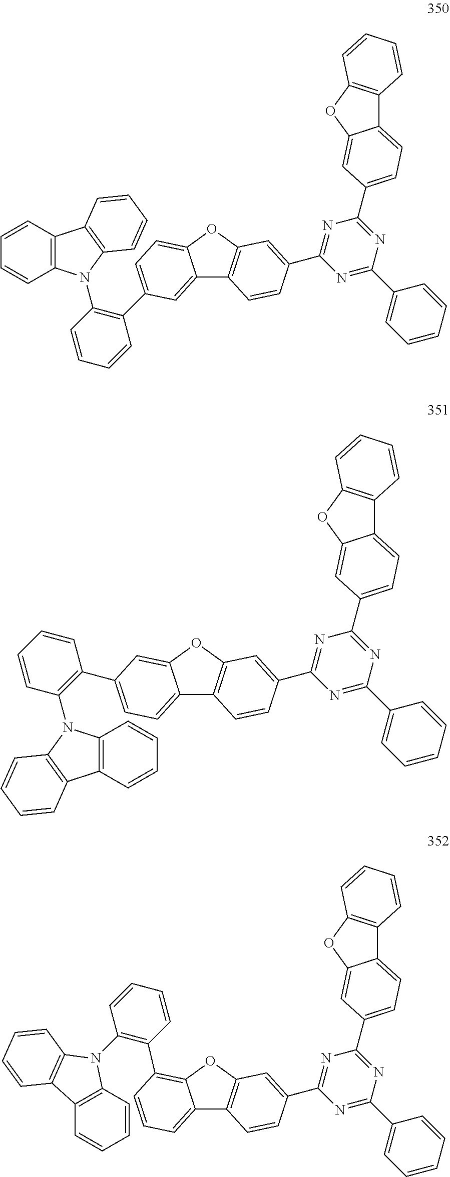 [SMILES: c1ccc(-c2nc(-c3ccc4c(c3)oc3ccccc34)nc(-c3ccc4c(c3)oc3ccc(-c5ccccc5-n5c6ccccc6c6ccccc65)cc34)n2)cc1 ; c1ccc(-c2nc(-c3ccc4c(c3)oc3ccccc34)nc(-c3ccc4c(c3)oc3cc(-c5ccccc5-n5c6ccccc6c6ccccc65)ccc34)n2)cc1 ; c1ccc(-c2nc(-c3ccc4c(c3)oc3ccccc34)nc(-c3ccc4c(c3)oc3c(-c5ccccc5-n5c6ccccc6c6ccccc65)cccc34)n2)cc1]

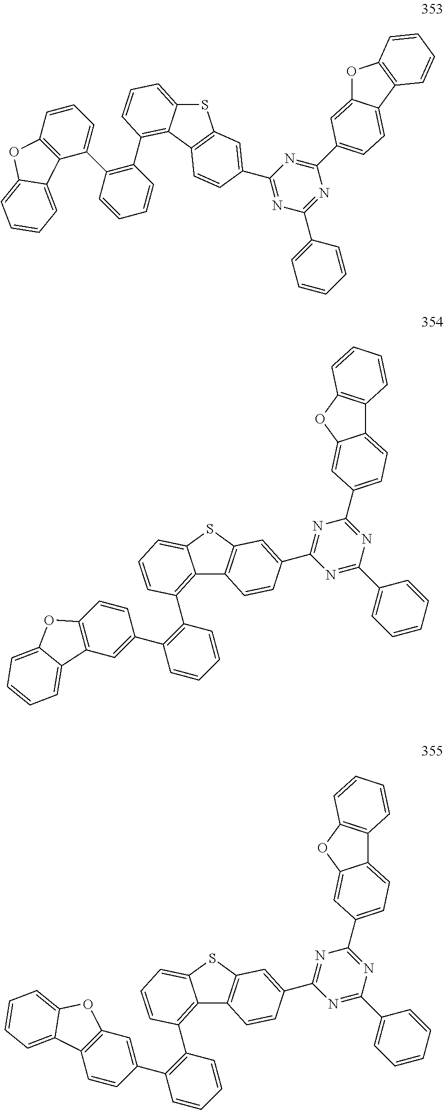 [SMILES: c1ccc(-c2nc(-c3ccc4c(c3)oc3ccccc34)nc(-c3ccc4c(c3)sc3cccc(-c5ccccc5-c5cccc6oc7ccccc7c56)c34)n2)cc1 ; c1ccc(-c2nc(-c3ccc4c(c3)oc3ccccc34)nc(-c3ccc4c(c3)sc3cccc(-c5ccccc5-c5ccc6oc7ccccc7c6c5)c34)n2)cc1 ; c1ccc(-c2nc(-c3ccc4c(c3)oc3ccccc34)nc(-c3ccc4c(c3)sc3cccc(-c5ccccc5-c5ccc6c(c5)oc5ccccc56)c34)n2)cc1]

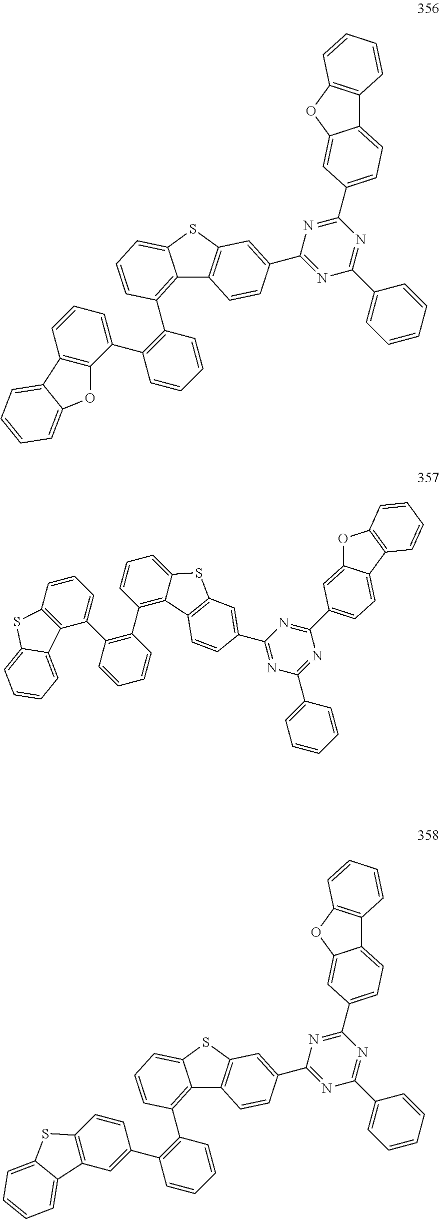 [SMILES: c1ccc(-c2nc(-c3ccc4c(c3)oc3ccccc34)nc(-c3ccc4c(c3)sc3cccc(-c5ccccc5-c5cccc6c5oc5ccccc56)c34)n2)cc1 ; c1ccc(-c2nc(-c3ccc4c(c3)oc3ccccc34)nc(-c3ccc4c(c3)sc3cccc(-c5ccccc5-c5cccc6sc7ccccc7c56)c34)n2)cc1 ; c1ccc(-c2nc(-c3ccc4c(c3)oc3ccccc34)nc(-c3ccc4c(c3)sc3cccc(-c5ccccc5-c5ccc6sc7ccccc7c6c5)c34)n2)cc1]

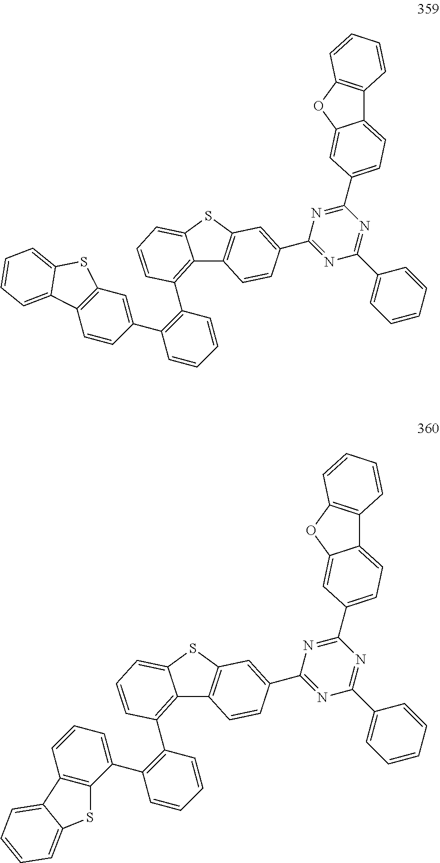 [SMILES: c1ccc(-c2nc(-c3ccc4c(c3)oc3ccccc34)nc(-c3ccc4c(c3)sc3cccc(-c5ccccc5-c5ccc6c(c5)sc5ccccc56)c34)n2)cc1 ; c1ccc(-c2nc(-c3ccc4c(c3)oc3ccccc34)nc(-c3ccc4c(c3)sc3cccc(-c5ccccc5-c5cccc6c5sc5ccccc56)c34)n2)cc1]

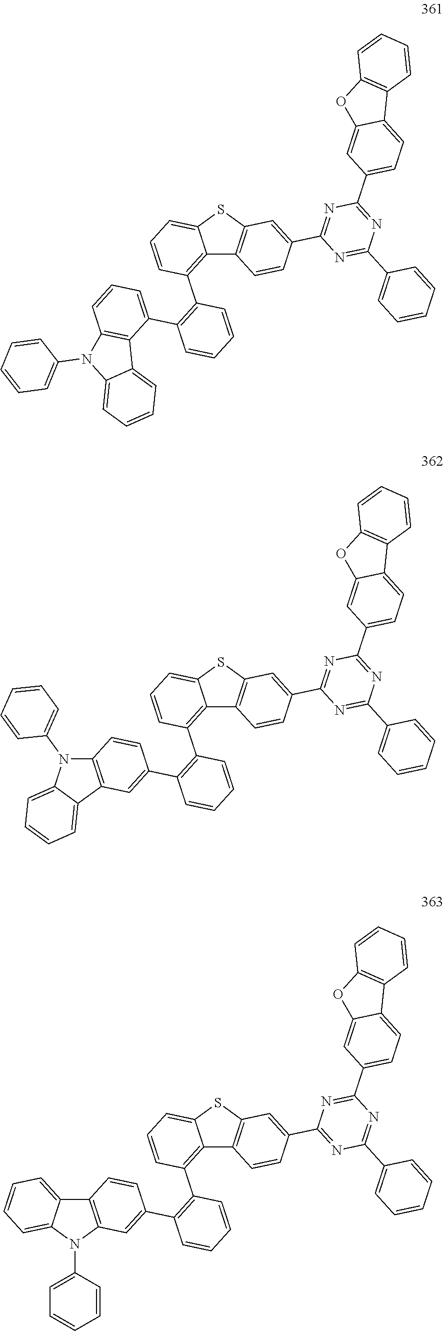 [SMILES: c1ccc(-c2nc(-c3ccc4c(c3)oc3ccccc34)nc(-c3ccc4c(c3)sc3cccc(-c5ccccc5-c5cccc6c5c5ccccc5n6-c5ccccc5)c34)n2)cc1 ; c1ccc(-c2nc(-c3ccc4c(c3)oc3ccccc34)nc(-c3ccc4c(c3)sc3cccc(-c5ccccc5-c5ccc6c(c5)c5ccccc5n6-c5ccccc5)c34)n2)cc1 ; c1ccc(-c2nc(-c3ccc4c(c3)oc3ccccc34)nc(-c3ccc4c(c3)sc3cccc(-c5ccccc5-c5ccc6c7ccccc7n(-c7ccccc7)c6c5)c34)n2)cc1]

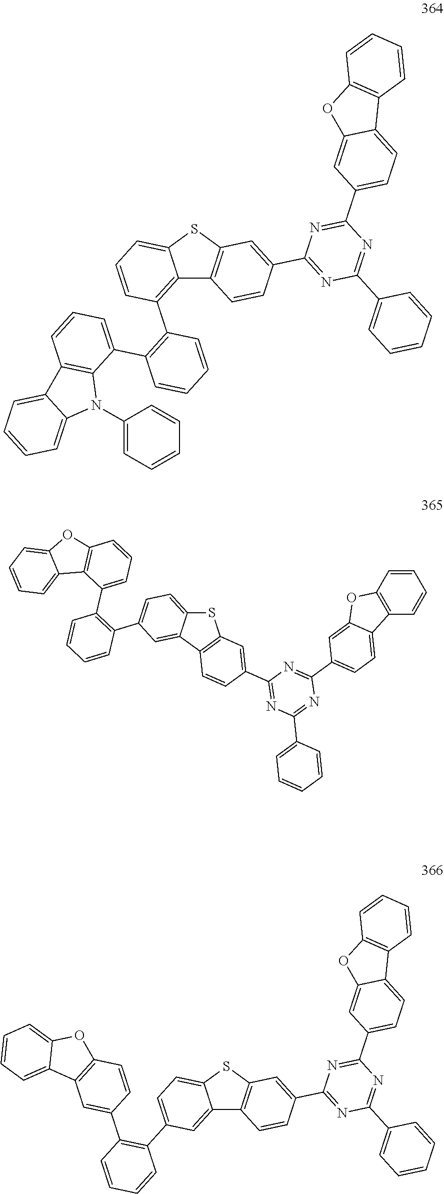 [SMILES: c1ccc(-c2nc(-c3ccc4c(c3)oc3ccccc34)nc(-c3ccc4c(c3)sc3cccc(-c5ccccc5-c5cccc6c7ccccc7n(-c7ccccc7)c56)c34)n2)cc1 ; c1ccc(-c2nc(-c3ccc4c(c3)oc3ccccc34)nc(-c3ccc4c(c3)sc3ccc(-c5ccccc5-c5cccc6oc7ccccc7c56)cc34)n2)cc1 ; c1ccc(-c2nc(-c3ccc4c(c3)oc3ccccc34)nc(-c3ccc4c(c3)sc3ccc(-c5ccccc5-c5ccc6oc7ccccc7c6c5)cc34)n2)cc1]

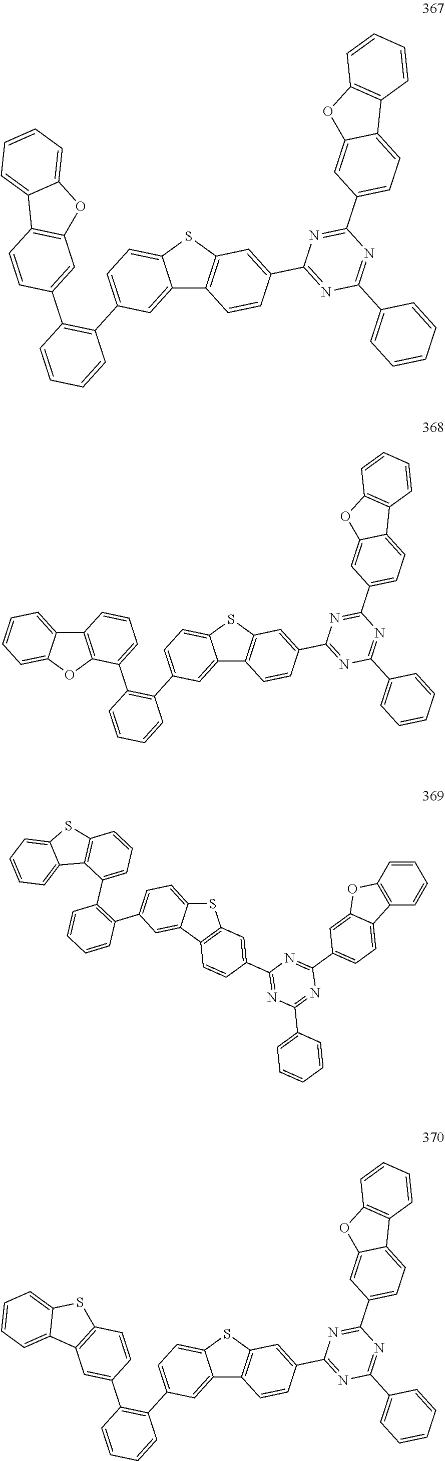 [SMILES: c1ccc(-c2nc(-c3ccc4c(c3)oc3ccccc34)nc(-c3ccc4c(c3)sc3ccc(-c5ccccc5-c5ccc6c(c5)oc5ccccc56)cc34)n2)cc1 ; c1ccc(-c2nc(-c3ccc4c(c3)oc3ccccc34)nc(-c3ccc4c(c3)sc3ccc(-c5ccccc5-c5cccc6c5oc5ccccc56)cc34)n2)cc1 ; c1ccc(-c2nc(-c3ccc4c(c3)oc3ccccc34)nc(-c3ccc4c(c3)sc3ccc(-c5ccccc5-c5cccc6sc7ccccc7c56)cc34)n2)cc1 ; c1ccc(-c2nc(-c3ccc4c(c3)oc3ccccc34)nc(-c3ccc4c(c3)sc3ccc(-c5ccccc5-c5ccc6sc7ccccc7c6c5)cc34)n2)cc1]

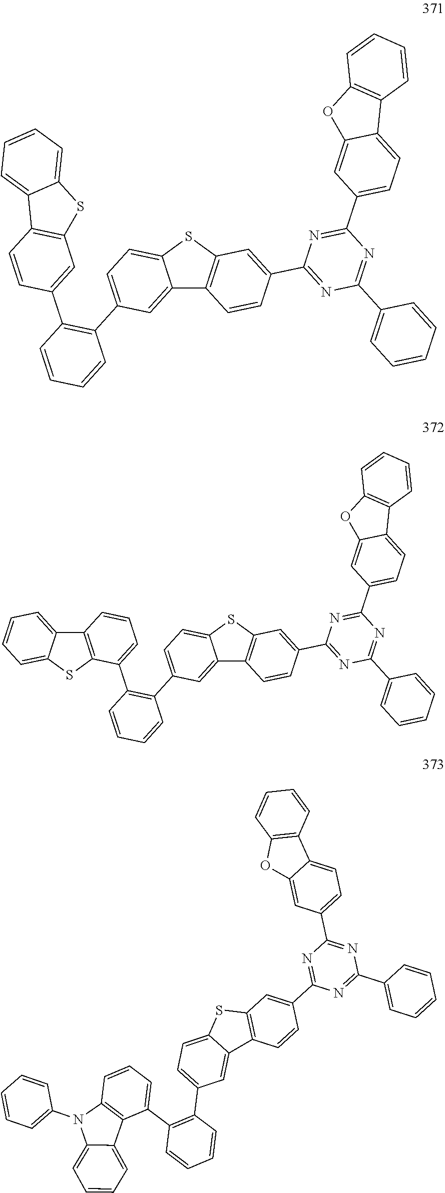 [SMILES: c1ccc(-c2nc(-c3ccc4c(c3)oc3ccccc34)nc(-c3ccc4c(c3)sc3ccc(-c5ccccc5-c5ccc6c(c5)sc5ccccc56)cc34)n2)cc1 ; c1ccc(-c2nc(-c3ccc4c(c3)oc3ccccc34)nc(-c3ccc4c(c3)sc3ccc(-c5ccccc5-c5cccc6c5sc5ccccc56)cc34)n2)cc1 ; c1ccc(-c2nc(-c3ccc4c(c3)oc3ccccc34)nc(-c3ccc4c(c3)sc3ccc(-c5ccccc5-c5cccc6c5c5ccccc5n6-c5ccccc5)cc34)n2)cc1]

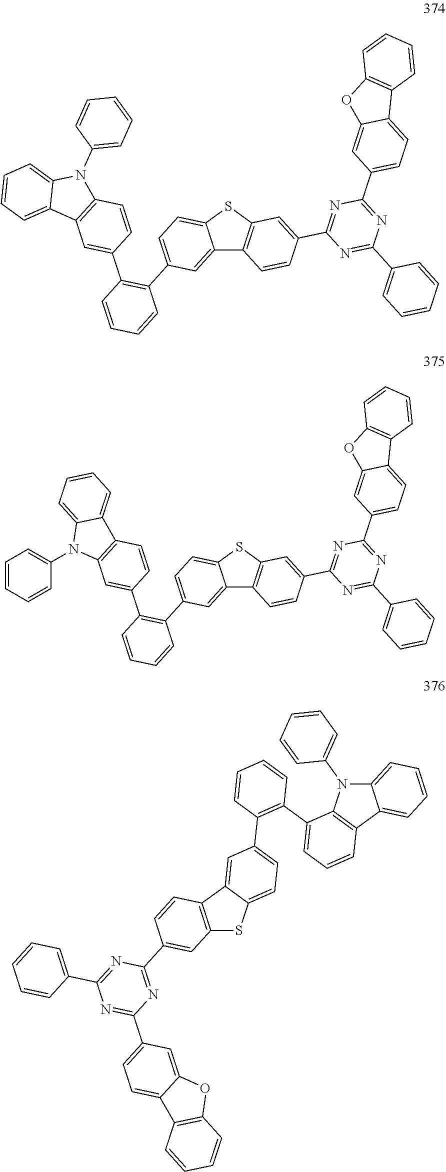 [SMILES: c1ccc(-c2nc(-c3ccc4c(c3)oc3ccccc34)nc(-c3ccc4c(c3)sc3ccc(-c5ccccc5-c5ccc6c(c5)c5ccccc5n6-c5ccccc5)cc34)n2)cc1 ; c1ccc(-c2nc(-c3ccc4c(c3)oc3ccccc34)nc(-c3ccc4c(c3)sc3ccc(-c5ccccc5-c5ccc6c7ccccc7n(-c7ccccc7)c6c5)cc34)n2)cc1 ; c1ccc(-c2nc(-c3ccc4c(c3)oc3ccccc34)nc(-c3ccc4c(c3)sc3ccc(-c5ccccc5-c5cccc6c7ccccc7n(-c7ccccc7)c56)cc34)n2)cc1]

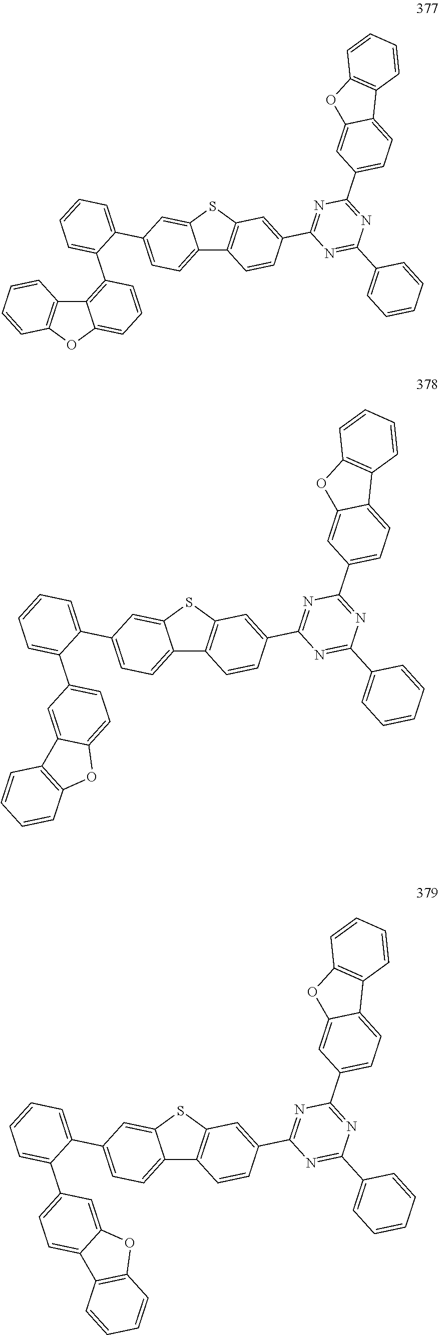 [SMILES: c1ccc(-c2nc(-c3ccc4c(c3)oc3ccccc34)nc(-c3ccc4c(c3)sc3cc(-c5ccccc5-c5cccc6oc7ccccc7c56)ccc34)n2)cc1 ; c1ccc(-c2nc(-c3ccc4c(c3)oc3ccccc34)nc(-c3ccc4c(c3)sc3cc(-c5ccccc5-c5ccc6oc7ccccc7c6c5)ccc34)n2)cc1 ; c1ccc(-c2nc(-c3ccc4c(c3)oc3ccccc34)nc(-c3ccc4c(c3)sc3cc(-c5ccccc5-c5ccc6c(c5)oc5ccccc56)ccc34)n2)cc1]

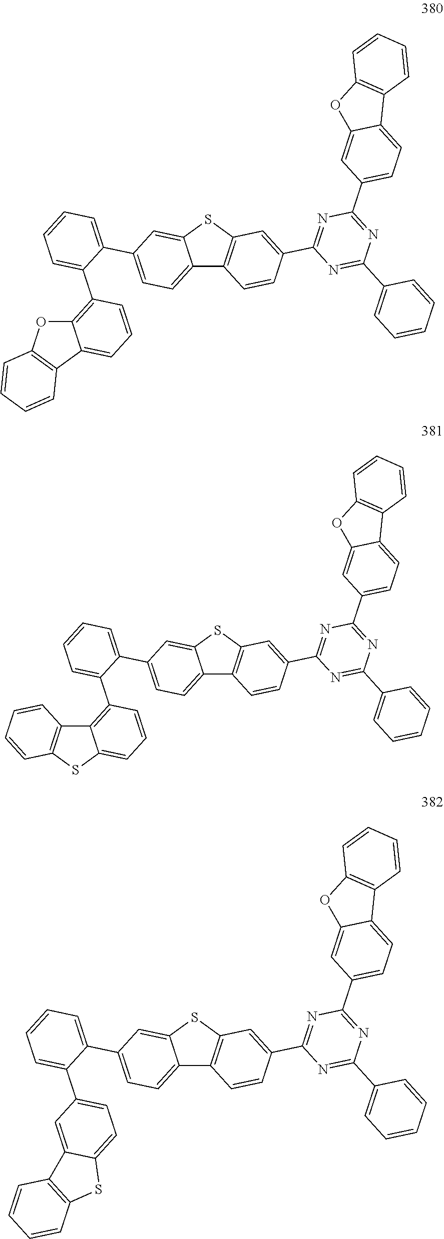 [SMILES: c1ccc(-c2nc(-c3ccc4c(c3)oc3ccccc34)nc(-c3ccc4c(c3)sc3cc(-c5ccccc5-c5cccc6c5oc5ccccc56)ccc34)n2)cc1 ; c1ccc(-c2nc(-c3ccc4c(c3)oc3ccccc34)nc(-c3ccc4c(c3)sc3cc(-c5ccccc5-c5cccc6sc7ccccc7c56)ccc34)n2)cc1 ; c1ccc(-c2nc(-c3ccc4c(c3)oc3ccccc34)nc(-c3ccc4c(c3)sc3cc(-c5ccccc5-c5ccc6sc7ccccc7c6c5)ccc34)n2)cc1]

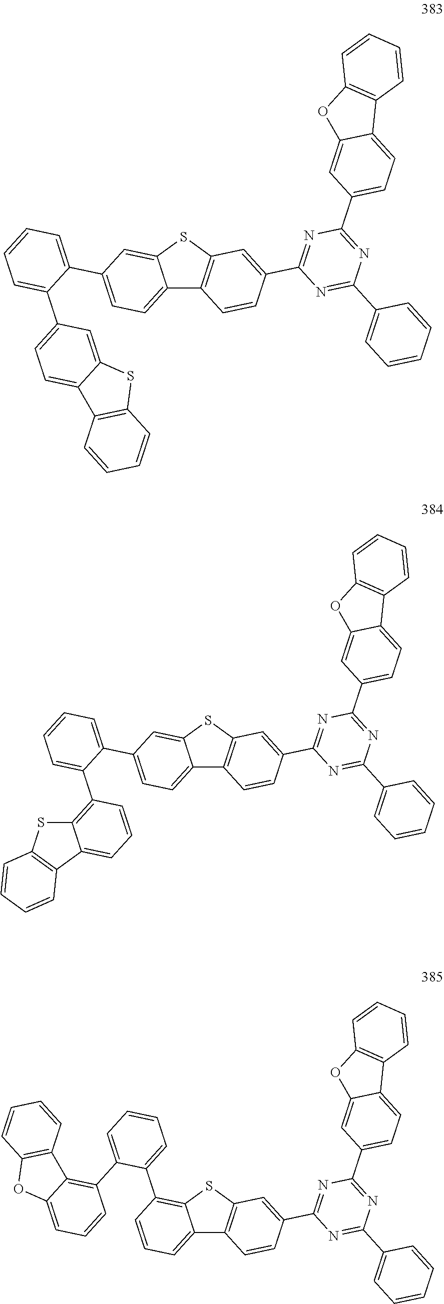 [SMILES: c1ccc(-c2nc(-c3ccc4c(c3)oc3ccccc34)nc(-c3ccc4c(c3)sc3cc(-c5ccccc5-c5ccc6c(c5)sc5ccccc56)ccc34)n2)cc1 ; c1ccc(-c2nc(-c3ccc4c(c3)oc3ccccc34)nc(-c3ccc4c(c3)sc3cc(-c5ccccc5-c5cccc6c5sc5ccccc56)ccc34)n2)cc1 ; c1ccc(-c2nc(-c3ccc4c(c3)oc3ccccc34)nc(-c3ccc4c(c3)sc3c(-c5ccccc5-c5cccc6oc7ccccc7c56)cccc34)n2)cc1]

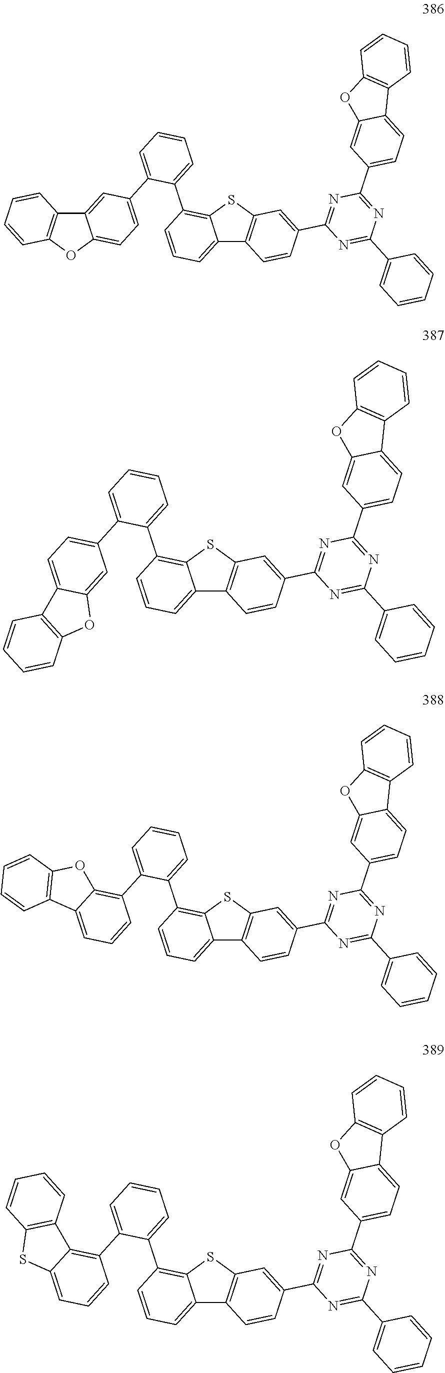 [SMILES: c1ccc(-c2nc(-c3ccc4c(c3)oc3ccccc34)nc(-c3ccc4c(c3)sc3c(-c5ccccc5-c5ccc6oc7ccccc7c6c5)cccc34)n2)cc1 ; c1ccc(-c2nc(-c3ccc4c(c3)oc3ccccc34)nc(-c3ccc4c(c3)sc3c(-c5ccccc5-c5ccc6c(c5)oc5ccccc56)cccc34)n2)cc1 ; c1ccc(-c2nc(-c3ccc4c(c3)oc3ccccc34)nc(-c3ccc4c(c3)sc3c(-c5ccccc5-c5cccc6c5oc5ccccc56)cccc34)n2)cc1 ; c1ccc(-c2nc(-c3ccc4c(c3)oc3ccccc34)nc(-c3ccc4c(c3)sc3c(-c5ccccc5-c5cccc6sc7ccccc7c56)cccc34)n2)cc1]

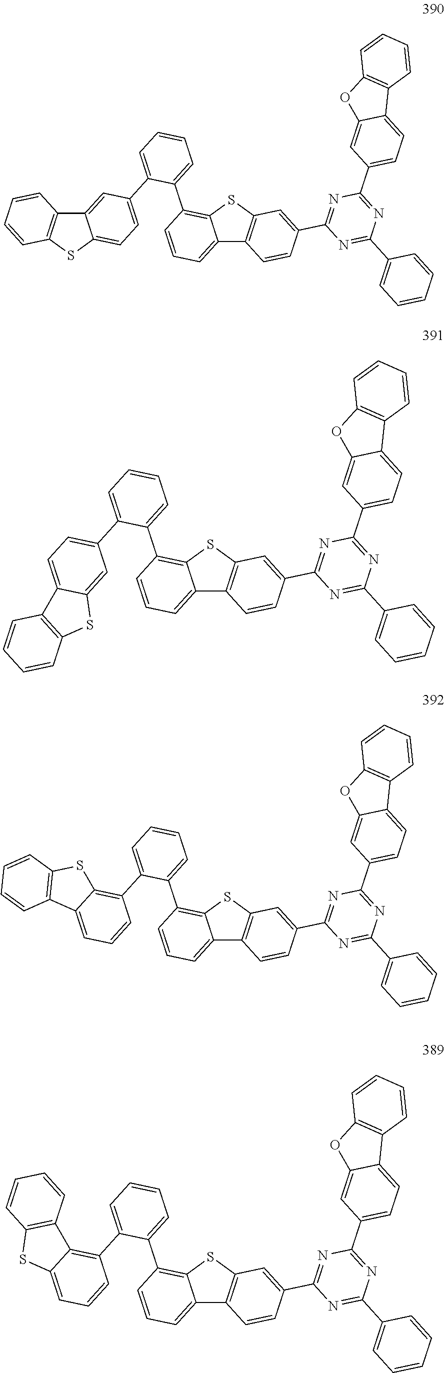 [SMILES: c1ccc(-c2nc(-c3ccc4c(c3)oc3ccccc34)nc(-c3ccc4c(c3)sc3c(-c5ccccc5-c5ccc6sc7ccccc7c6c5)cccc34)n2)cc1 ; c1ccc(-c2nc(-c3ccc4c(c3)oc3ccccc34)nc(-c3ccc4c(c3)sc3c(-c5ccccc5-c5ccc6c(c5)sc5ccccc56)cccc34)n2)cc1 ; c1ccc(-c2nc(-c3ccc4c(c3)oc3ccccc34)nc(-c3ccc4c(c3)sc3c(-c5ccccc5-c5cccc6c5sc5ccccc56)cccc34)n2)cc1 ; c1ccc(-c2nc(-c3ccc4c(c3)oc3ccccc34)nc(-c3ccc4c(c3)sc3c(-c5ccccc5-c5cccc6sc7ccccc7c56)cccc34)n2)cc1]

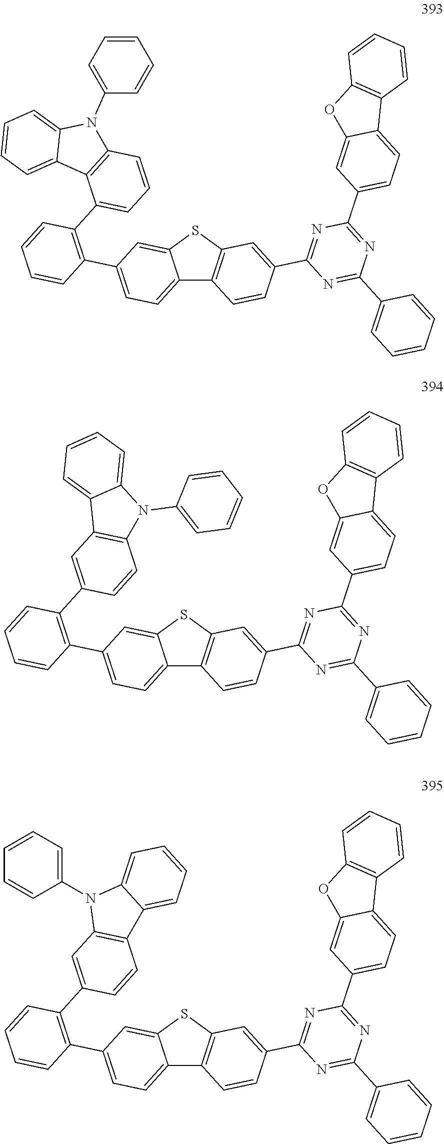 [SMILES: c1ccc(-c2nc(-c3ccc4c(c3)oc3ccccc34)nc(-c3ccc4c(c3)sc3cc(-c5ccccc5-c5cccc6c5c5ccccc5n6-c5ccccc5)ccc34)n2)cc1 ; c1ccc(-c2nc(-c3ccc4c(c3)oc3ccccc34)nc(-c3ccc4c(c3)sc3cc(-c5ccccc5-c5ccc6c(c5)c5ccccc5n6-c5ccccc5)ccc34)n2)cc1 ; c1ccc(-c2nc(-c3ccc4c(c3)oc3ccccc34)nc(-c3ccc4c(c3)sc3cc(-c5ccccc5-c5ccc6c7ccccc7n(-c7ccccc7)c6c5)ccc34)n2)cc1]

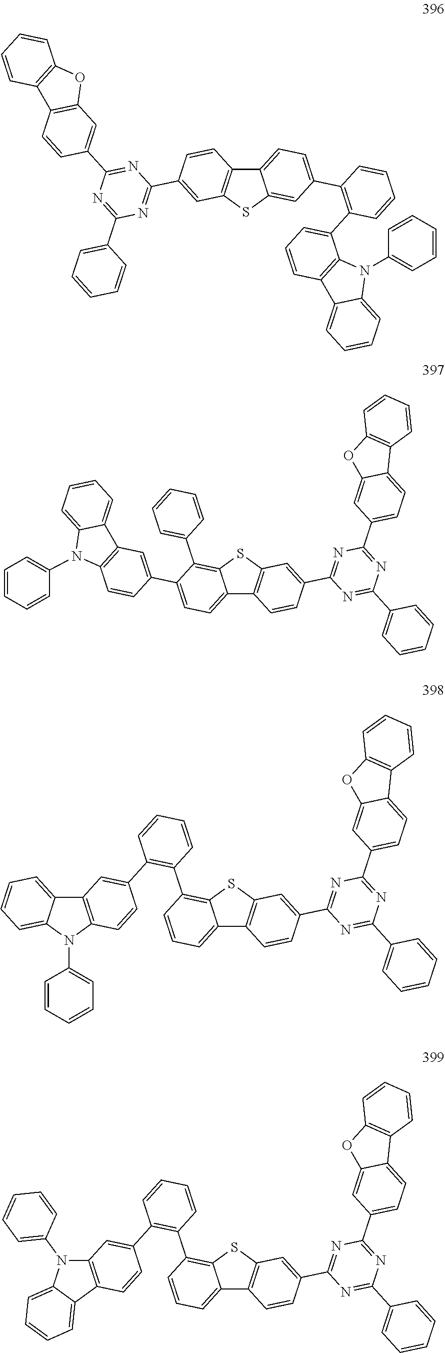 [SMILES: c1ccc(-c2nc(-c3ccc4c(c3)oc3ccccc34)nc(-c3ccc4c(c3)sc3cc(-c5ccccc5-c5cccc6c7ccccc7n(-c7ccccc7)c56)ccc34)n2)cc1 ; c1ccc(-c2nc(-c3ccc4c(c3)oc3ccccc34)nc(-c3ccc4c(c3)sc3c(-c5ccccc5)c(-c5ccc6c(c5)c5ccccc5n6-c5ccccc5)ccc34)n2)cc1 ; c1ccc(-c2nc(-c3ccc4c(c3)oc3ccccc34)nc(-c3ccc4c(c3)sc3c(-c5ccccc5-c5ccc6c(c5)c5ccccc5n6-c5ccccc5)cccc34)n2)cc1 ; c1ccc(-c2nc(-c3ccc4c(c3)oc3ccccc34)nc(-c3ccc4c(c3)sc3c(-c5ccccc5-c5ccc6c7ccccc7n(-c7ccccc7)c6c5)cccc34)n2)cc1]

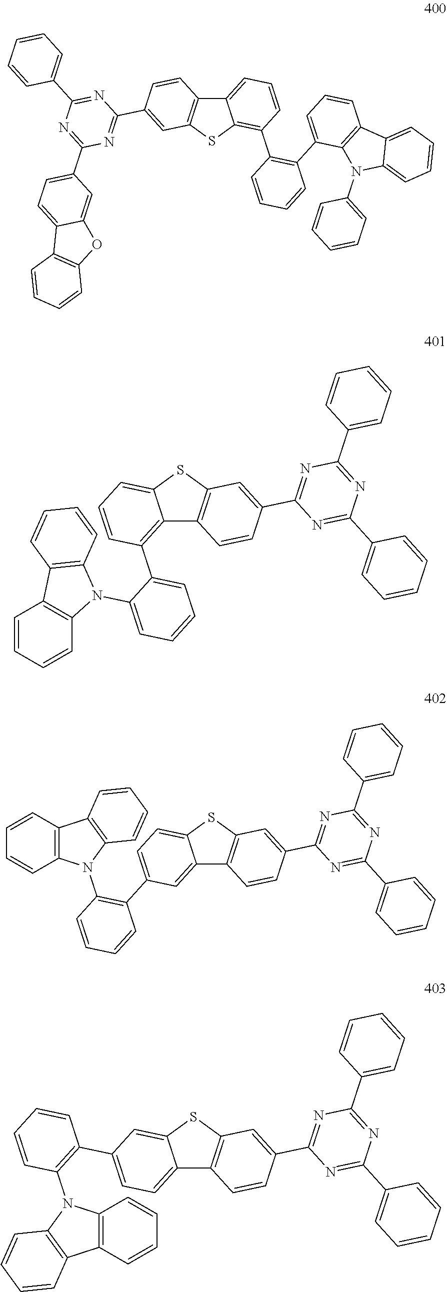 [SMILES: c1ccc(-c2nc(-c3ccc4c(c3)oc3ccccc34)nc(-c3ccc4c(c3)sc3c(-c5ccccc5-c5cccc6c7ccccc7n(-c7ccccc7)c56)cccc34)n2)cc1 ; c1ccc(-c2nc(-c3ccccc3)nc(-c3ccc4c(c3)sc3cccc(-c5ccccc5-n5c6ccccc6c6ccccc65)c34)n2)cc1 ; c1ccc(-c2nc(-c3ccccc3)nc(-c3ccc4c(c3)sc3ccc(-c5ccccc5-n5c6ccccc6c6ccccc65)cc34)n2)cc1 ; c1ccc(-c2nc(-c3ccccc3)nc(-c3ccc4c(c3)sc3cc(-c5ccccc5-n5c6ccccc6c6ccccc65)ccc34)n2)cc1]

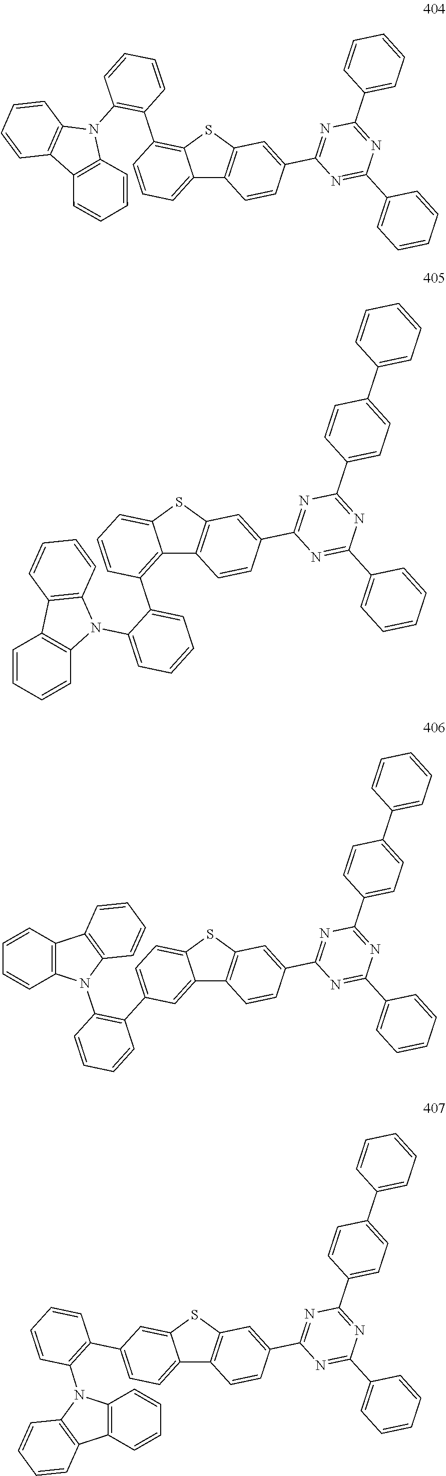 [SMILES: c1ccc(-c2nc(-c3ccccc3)nc(-c3ccc4c(c3)sc3c(-c5ccccc5-n5c6ccccc6c6ccccc65)cccc34)n2)cc1 ; c1ccc(-c2ccc(-c3nc(-c4ccccc4)nc(-c4ccc5c(c4)sc4cccc(-c6ccccc6-n6c7ccccc7c7ccccc76)c45)n3)cc2)cc1 ; c1ccc(-c2ccc(-c3nc(-c4ccccc4)nc(-c4ccc5c(c4)sc4ccc(-c6ccccc6-n6c7ccccc7c7ccccc76)cc45)n3)cc2)cc1 ; c1ccc(-c2ccc(-c3nc(-c4ccccc4)nc(-c4ccc5c(c4)sc4cc(-c6ccccc6-n6c7ccccc7c7ccccc76)ccc45)n3)cc2)cc1]

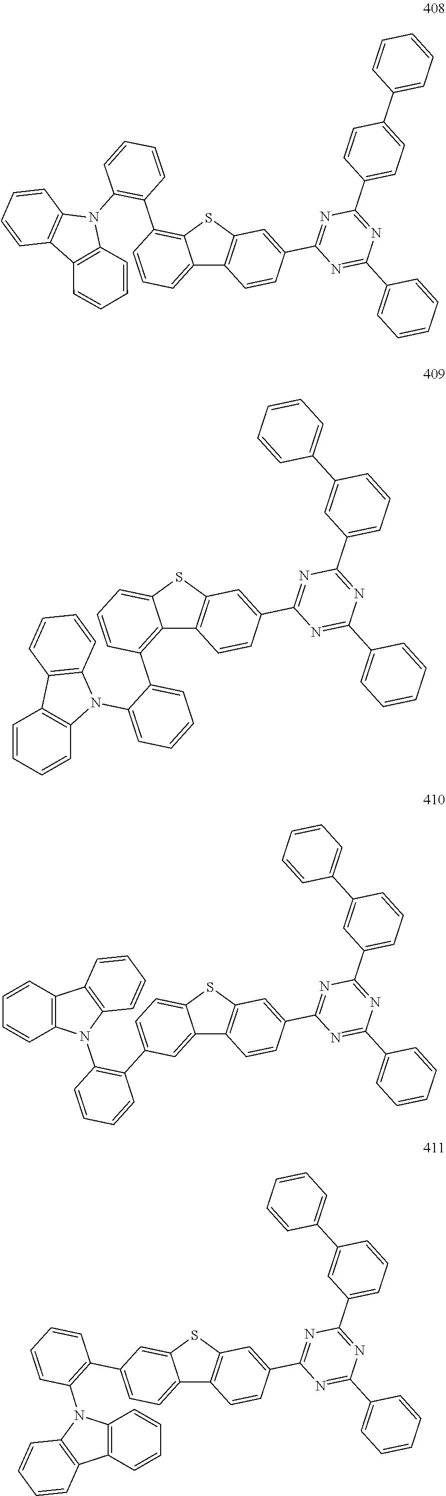 [SMILES: c1ccc(-c2ccc(-c3nc(-c4ccccc4)nc(-c4ccc5c(c4)sc4c(-c6ccccc6-n6c7ccccc7c7ccccc76)cccc45)n3)cc2)cc1 ; c1ccc(-c2cccc(-c3nc(-c4ccccc4)nc(-c4ccc5c(c4)sc4cccc(-c6ccccc6-n6c7ccccc7c7ccccc76)c45)n3)c2)cc1 ; c1ccc(-c2cccc(-c3nc(-c4ccccc4)nc(-c4ccc5c(c4)sc4ccc(-c6ccccc6-n6c7ccccc7c7ccccc76)cc45)n3)c2)cc1 ; c1ccc(-c2cccc(-c3nc(-c4ccccc4)nc(-c4ccc5c(c4)sc4cc(-c6ccccc6-n6c7ccccc7c7ccccc76)ccc45)n3)c2)cc1]

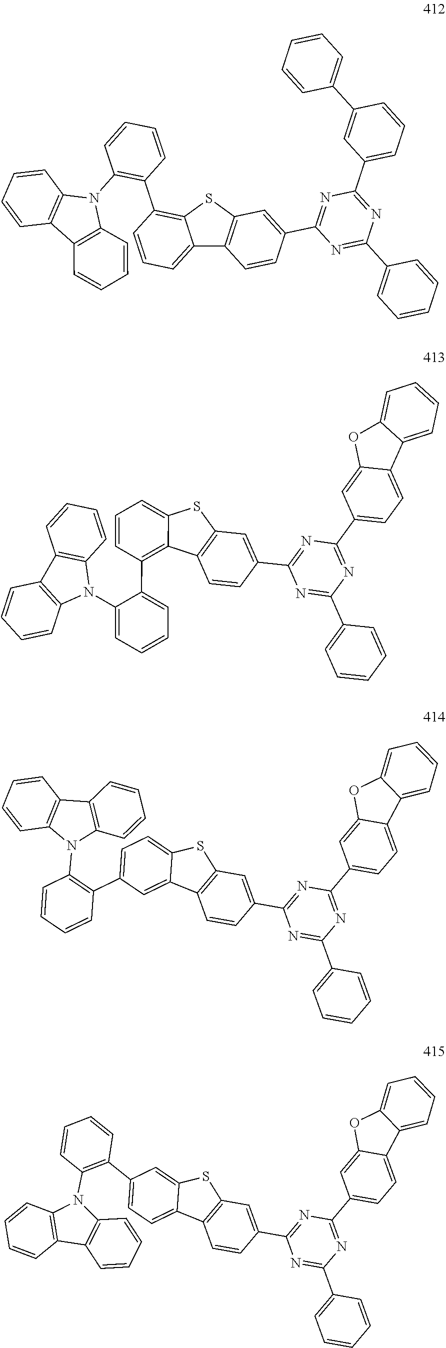 [SMILES: c1ccc(-c2cccc(-c3nc(-c4ccccc4)nc(-c4ccc5c(c4)sc4c(-c6ccccc6-n6c7ccccc7c7ccccc76)cccc45)n3)c2)cc1 ; c1ccc(-c2nc(-c3ccc4c(c3)oc3ccccc34)nc(-c3ccc4c(c3)sc3cccc(-c5ccccc5-n5c6ccccc6c6ccccc65)c34)n2)cc1 ; c1ccc(-c2nc(-c3ccc4c(c3)oc3ccccc34)nc(-c3ccc4c(c3)sc3ccc(-c5ccccc5-n5c6ccccc6c6ccccc65)cc34)n2)cc1 ; c1ccc(-c2nc(-c3ccc4c(c3)oc3ccccc34)nc(-c3ccc4c(c3)sc3cc(-c5ccccc5-n5c6ccccc6c6ccccc65)ccc34)n2)cc1]

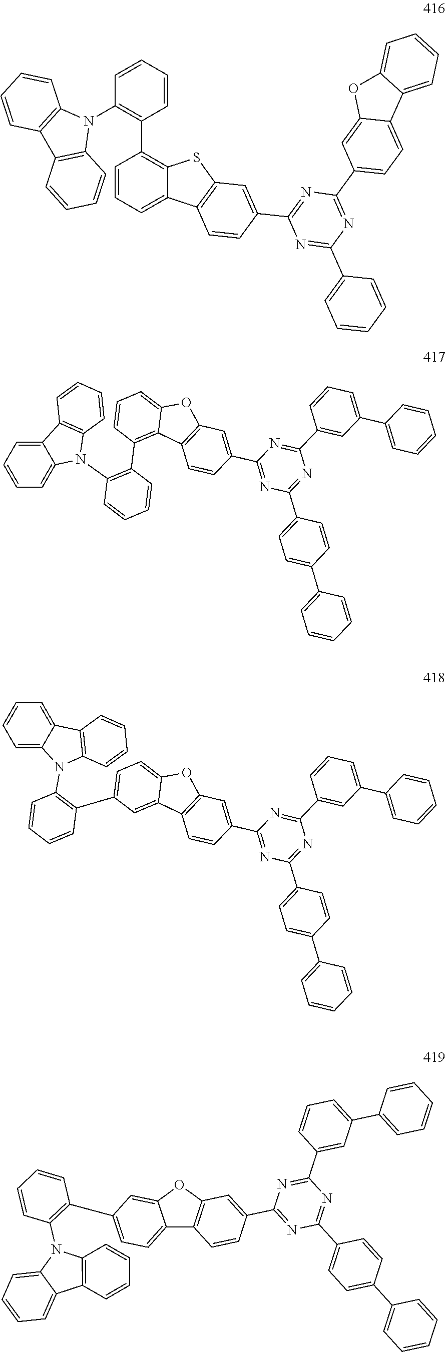 [SMILES: c1ccc(-c2nc(-c3ccc4c(c3)oc3ccccc34)nc(-c3ccc4c(c3)sc3c(-c5ccccc5-n5c6ccccc6c6ccccc65)cccc34)n2)cc1 ; c1ccc(-c2ccc(-c3nc(-c4cccc(-c5ccccc5)c4)nc(-c4ccc5c(c4)oc4cccc(-c6ccccc6-n6c7ccccc7c7ccccc76)c45)n3)cc2)cc1 ; c1ccc(-c2ccc(-c3nc(-c4cccc(-c5ccccc5)c4)nc(-c4ccc5c(c4)oc4ccc(-c6ccccc6-n6c7ccccc7c7ccccc76)cc45)n3)cc2)cc1 ; c1ccc(-c2ccc(-c3nc(-c4cccc(-c5ccccc5)c4)nc(-c4ccc5c(c4)oc4cc(-c6ccccc6-n6c7ccccc7c7ccccc76)ccc45)n3)cc2)cc1]

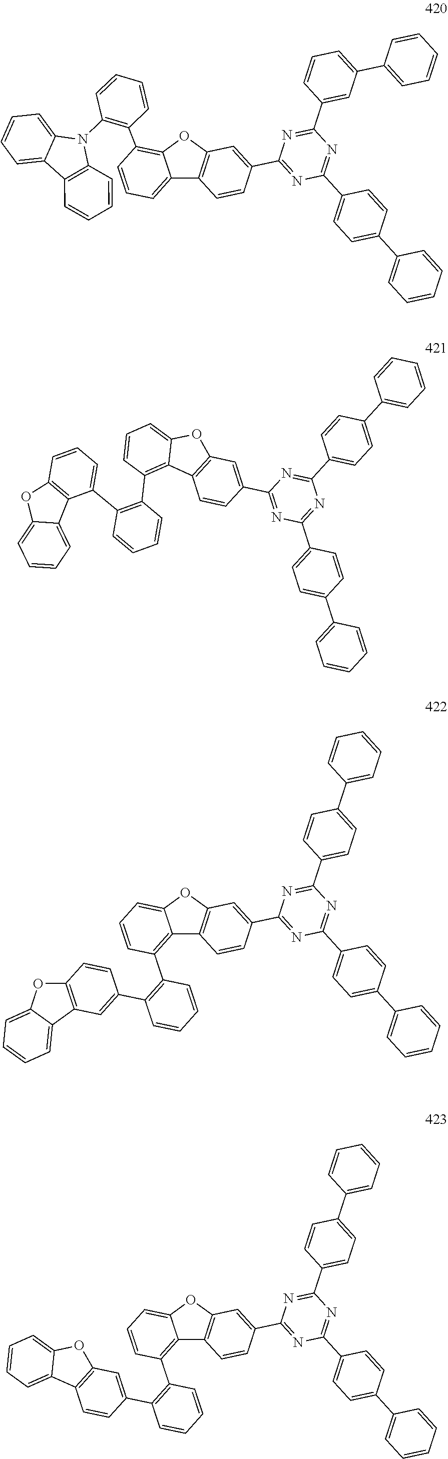 [SMILES: c1ccc(-c2ccc(-c3nc(-c4cccc(-c5ccccc5)c4)nc(-c4ccc5c(c4)oc4c(-c6ccccc6-n6c7ccccc7c7ccccc76)cccc45)n3)cc2)cc1 ; c1ccc(-c2ccc(-c3nc(-c4ccc(-c5ccccc5)cc4)nc(-c4ccc5c(c4)oc4cccc(-c6ccccc6-c6cccc7oc8ccccc8c67)c45)n3)cc2)cc1 ; c1ccc(-c2ccc(-c3nc(-c4ccc(-c5ccccc5)cc4)nc(-c4ccc5c(c4)oc4cccc(-c6ccccc6-c6ccc7oc8ccccc8c7c6)c45)n3)cc2)cc1 ; c1ccc(-c2ccc(-c3nc(-c4ccc(-c5ccccc5)cc4)nc(-c4ccc5c(c4)oc4cccc(-c6ccccc6-c6ccc7c(c6)oc6ccccc67)c45)n3)cc2)cc1]

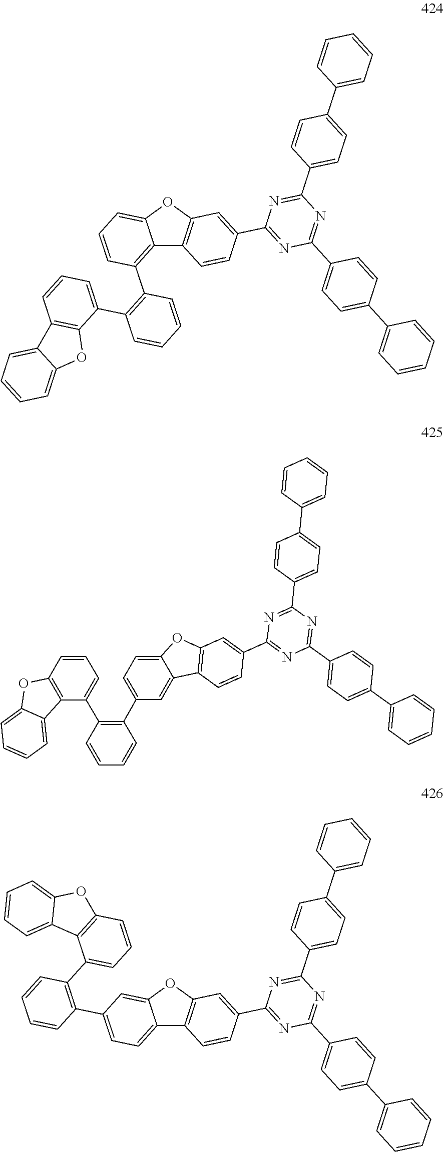 [SMILES: c1ccc(-c2ccc(-c3nc(-c4ccc(-c5ccccc5)cc4)nc(-c4ccc5c(c4)oc4cccc(-c6ccccc6-c6cccc7c6oc6ccccc67)c45)n3)cc2)cc1 ; c1ccc(-c2ccc(-c3nc(-c4ccc(-c5ccccc5)cc4)nc(-c4ccc5c(c4)oc4ccc(-c6ccccc6-c6cccc7oc8ccccc8c67)cc45)n3)cc2)cc1 ; c1ccc(-c2ccc(-c3nc(-c4ccc(-c5ccccc5)cc4)nc(-c4ccc5c(c4)oc4cc(-c6ccccc6-c6cccc7oc8ccccc8c67)ccc45)n3)cc2)cc1]

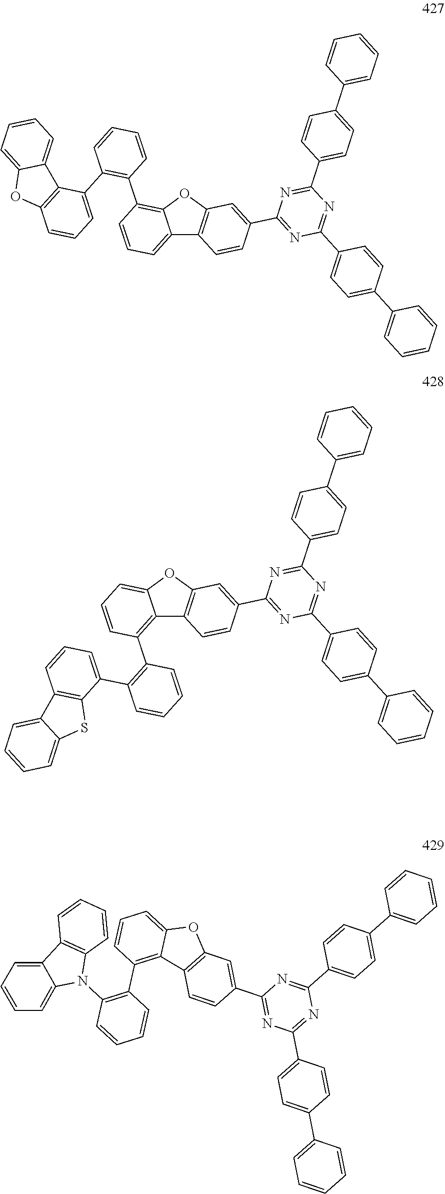 [SMILES: c1ccc(-c2ccc(-c3nc(-c4ccc(-c5ccccc5)cc4)nc(-c4ccc5c(c4)oc4c(-c6ccccc6-c6cccc7oc8ccccc8c67)cccc45)n3)cc2)cc1 ; c1ccc(-c2ccc(-c3nc(-c4ccc(-c5ccccc5)cc4)nc(-c4ccc5c(c4)oc4cccc(-c6ccccc6-c6cccc7c6sc6ccccc67)c45)n3)cc2)cc1 ; c1ccc(-c2ccc(-c3nc(-c4ccc(-c5ccccc5)cc4)nc(-c4ccc5c(c4)oc4cccc(-c6ccccc6-n6c7ccccc7c7ccccc76)c45)n3)cc2)cc1]

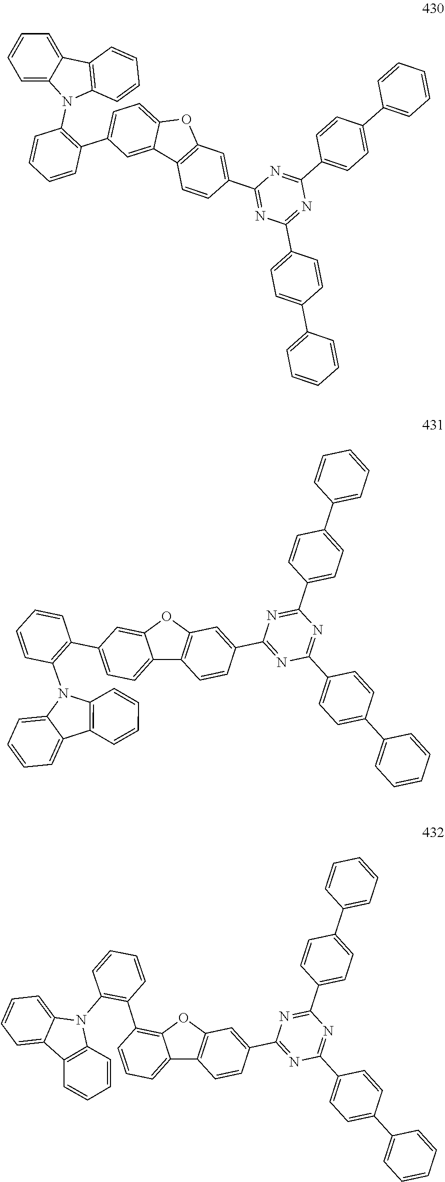 [SMILES: c1ccc(-c2ccc(-c3nc(-c4ccc(-c5ccccc5)cc4)nc(-c4ccc5c(c4)oc4ccc(-c6ccccc6-n6c7ccccc7c7ccccc76)cc45)n3)cc2)cc1 ; c1ccc(-c2ccc(-c3nc(-c4ccc(-c5ccccc5)cc4)nc(-c4ccc5c(c4)oc4cc(-c6ccccc6-n6c7ccccc7c7ccccc76)ccc45)n3)cc2)cc1 ; c1ccc(-c2ccc(-c3nc(-c4ccc(-c5ccccc5)cc4)nc(-c4ccc5c(c4)oc4c(-c6ccccc6-n6c7ccccc7c7ccccc76)cccc45)n3)cc2)cc1]

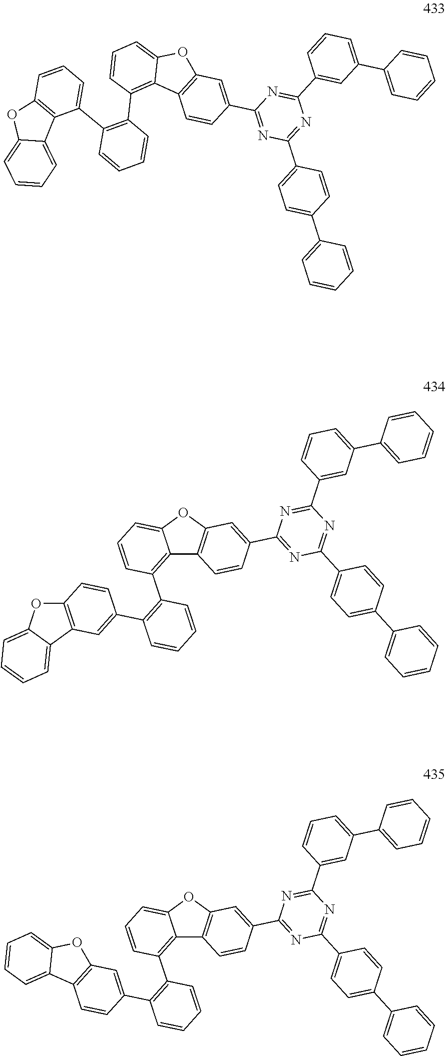 [SMILES: c1ccc(-c2ccc(-c3nc(-c4cccc(-c5ccccc5)c4)nc(-c4ccc5c(c4)oc4cccc(-c6ccccc6-c6cccc7oc8ccccc8c67)c45)n3)cc2)cc1 ; c1ccc(-c2ccc(-c3nc(-c4cccc(-c5ccccc5)c4)nc(-c4ccc5c(c4)oc4cccc(-c6ccccc6-c6ccc7oc8ccccc8c7c6)c45)n3)cc2)cc1 ; c1ccc(-c2ccc(-c3nc(-c4cccc(-c5ccccc5)c4)nc(-c4ccc5c(c4)oc4cccc(-c6ccccc6-c6ccc7c(c6)oc6ccccc67)c45)n3)cc2)cc1]

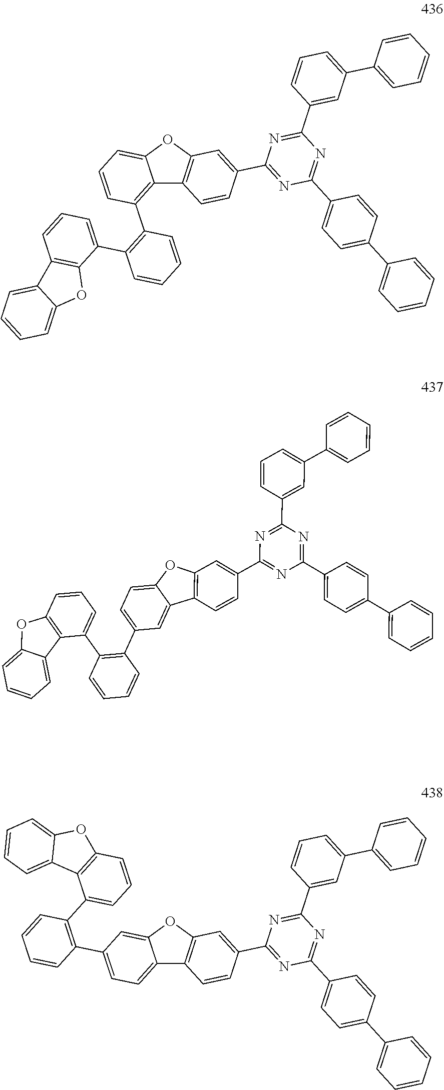 [SMILES: c1ccc(-c2ccc(-c3nc(-c4cccc(-c5ccccc5)c4)nc(-c4ccc5c(c4)oc4cccc(-c6ccccc6-c6cccc7c6oc6ccccc67)c45)n3)cc2)cc1 ; c1ccc(-c2ccc(-c3nc(-c4cccc(-c5ccccc5)c4)nc(-c4ccc5c(c4)oc4ccc(-c6ccccc6-c6cccc7oc8ccccc8c67)cc45)n3)cc2)cc1 ; c1ccc(-c2ccc(-c3nc(-c4cccc(-c5ccccc5)c4)nc(-c4ccc5c(c4)oc4cc(-c6ccccc6-c6cccc7oc8ccccc8c67)ccc45)n3)cc2)cc1]

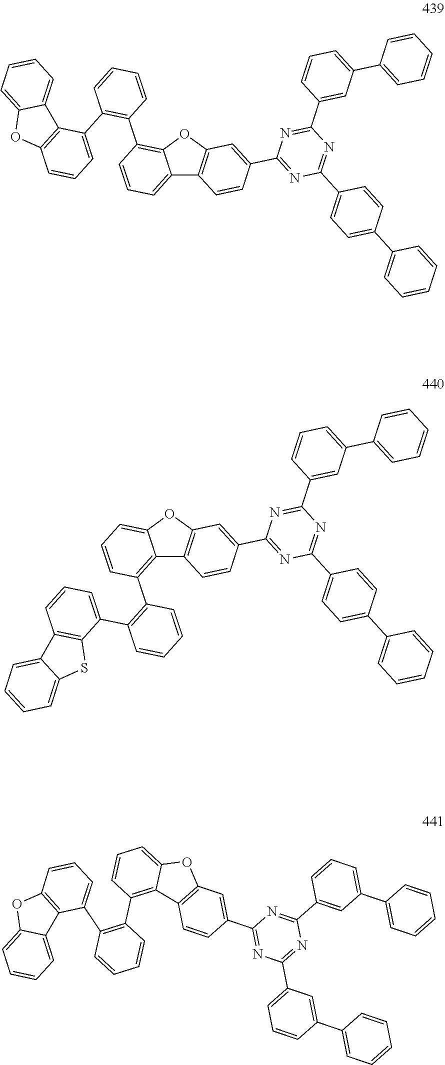 [SMILES: c1ccc(-c2ccc(-c3nc(-c4cccc(-c5ccccc5)c4)nc(-c4ccc5c(c4)oc4c(-c6ccccc6-c6cccc7oc8ccccc8c67)cccc45)n3)cc2)cc1 ; c1ccc(-c2ccc(-c3nc(-c4cccc(-c5ccccc5)c4)nc(-c4ccc5c(c4)oc4cccc(-c6ccccc6-c6cccc7c6sc6ccccc67)c45)n3)cc2)cc1 ; c1ccc(-c2cccc(-c3nc(-c4cccc(-c5ccccc5)c4)nc(-c4ccc5c(c4)oc4cccc(-c6ccccc6-c6cccc7oc8ccccc8c67)c45)n3)c2)cc1]

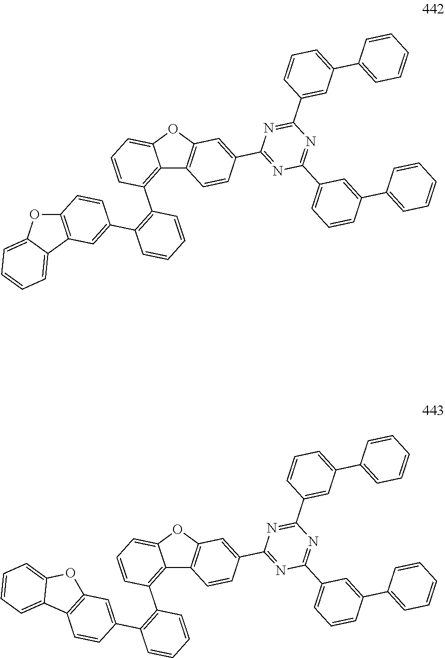 [SMILES: c1ccc(-c2cccc(-c3nc(-c4cccc(-c5ccccc5)c4)nc(-c4ccc5c(c4)oc4cccc(-c6ccccc6-c6ccc7oc8ccccc8c7c6)c45)n3)c2)cc1 ; c1ccc(-c2cccc(-c3nc(-c4cccc(-c5ccccc5)c4)nc(-c4ccc5c(c4)oc4cccc(-c6ccccc6-c6ccc7c(c6)oc6ccccc67)c45)n3)c2)cc1]

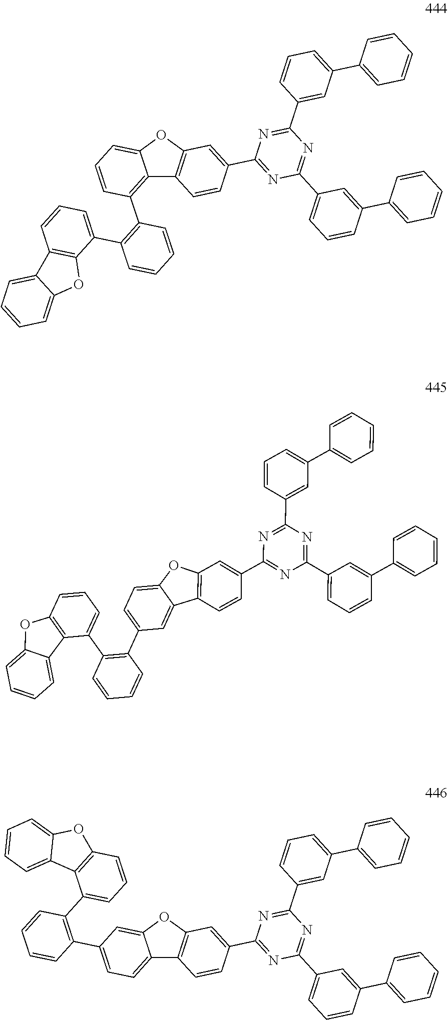 [SMILES: c1ccc(-c2cccc(-c3nc(-c4cccc(-c5ccccc5)c4)nc(-c4ccc5c(c4)oc4cccc(-c6ccccc6-c6cccc7c6oc6ccccc67)c45)n3)c2)cc1 ; c1ccc(-c2cccc(-c3nc(-c4cccc(-c5ccccc5)c4)nc(-c4ccc5c(c4)oc4ccc(-c6ccccc6-c6cccc7oc8ccccc8c67)cc45)n3)c2)cc1 ; c1ccc(-c2cccc(-c3nc(-c4cccc(-c5ccccc5)c4)nc(-c4ccc5c(c4)oc4cc(-c6ccccc6-c6cccc7oc8ccccc8c67)ccc45)n3)c2)cc1]

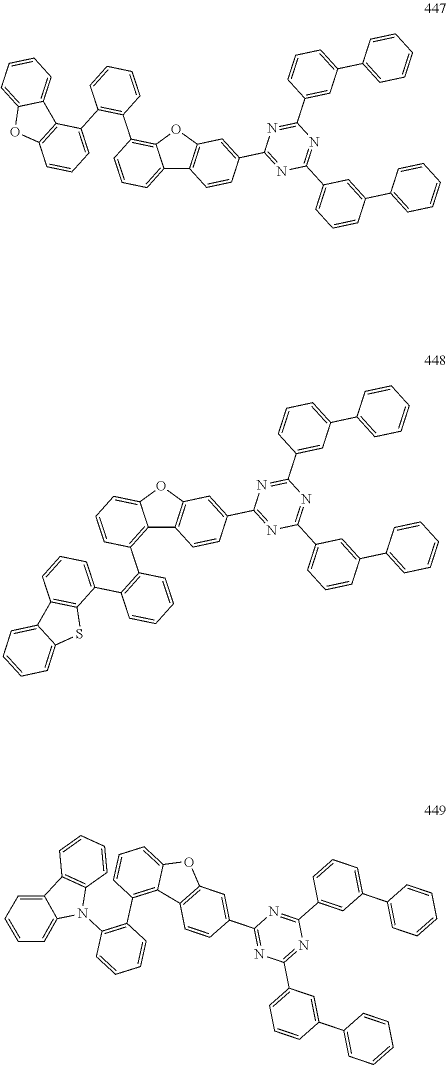 [SMILES: c1ccc(-c2cccc(-c3nc(-c4cccc(-c5ccccc5)c4)nc(-c4ccc5c(c4)oc4c(-c6ccccc6-c6cccc7oc8ccccc8c67)cccc45)n3)c2)cc1 ; c1ccc(-c2cccc(-c3nc(-c4cccc(-c5ccccc5)c4)nc(-c4ccc5c(c4)oc4cccc(-c6ccccc6-c6cccc7c6sc6ccccc67)c45)n3)c2)cc1 ; c1ccc(-c2cccc(-c3nc(-c4cccc(-c5ccccc5)c4)nc(-c4ccc5c(c4)oc4cccc(-c6ccccc6-n6c7ccccc7c7ccccc76)c45)n3)c2)cc1]

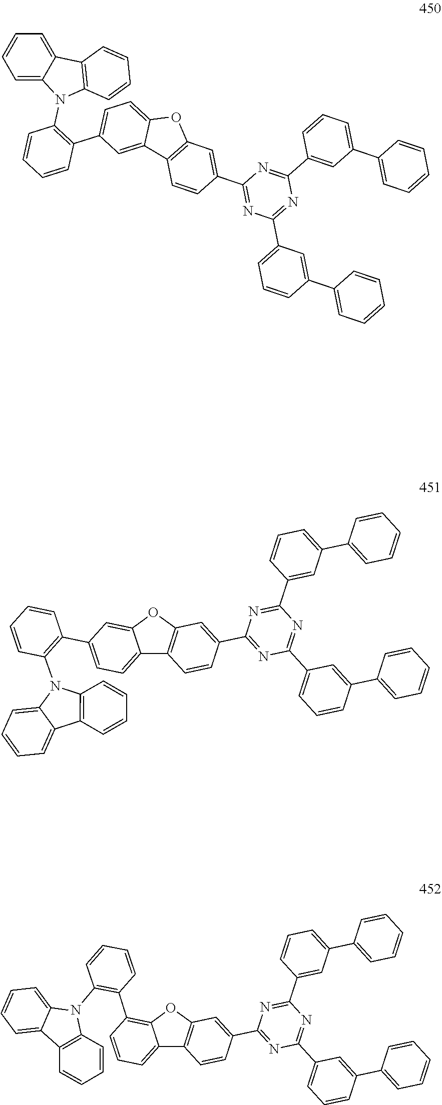 [SMILES: c1ccc(-c2cccc(-c3nc(-c4cccc(-c5ccccc5)c4)nc(-c4ccc5c(c4)oc4ccc(-c6ccccc6-n6c7ccccc7c7ccccc76)cc45)n3)c2)cc1 ; c1ccc(-c2cccc(-c3nc(-c4cccc(-c5ccccc5)c4)nc(-c4ccc5c(c4)oc4cc(-c6ccccc6-n6c7ccccc7c7ccccc76)ccc45)n3)c2)cc1 ; c1ccc(-c2cccc(-c3nc(-c4cccc(-c5ccccc5)c4)nc(-c4ccc5c(c4)oc4c(-c6ccccc6-n6c7ccccc7c7ccccc76)cccc45)n3)c2)cc1]

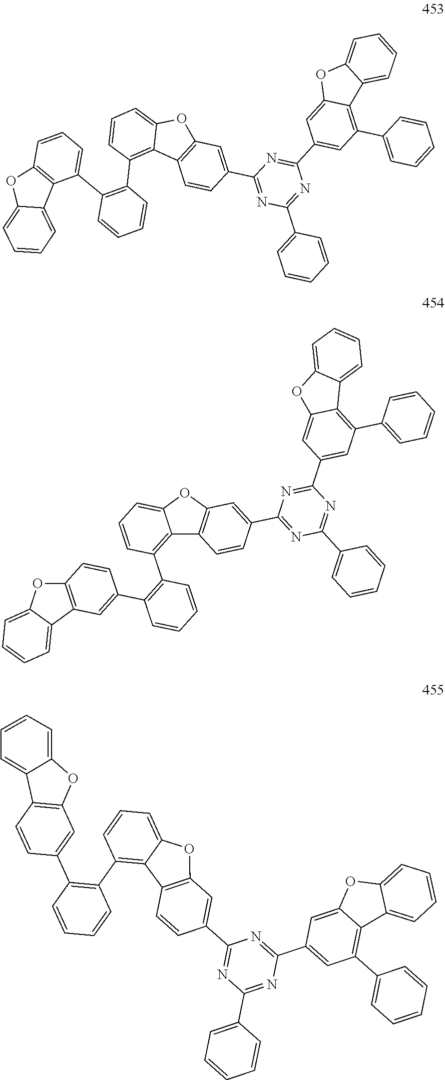 [SMILES: c1ccc(-c2nc(-c3ccc4c(c3)oc3cccc(-c5ccccc5-c5cccc6oc7ccccc7c56)c34)nc(-c3cc(-c4ccccc4)c4c(c3)oc3ccccc34)n2)cc1 ; c1ccc(-c2nc(-c3ccc4c(c3)oc3cccc(-c5ccccc5-c5ccc6oc7ccccc7c6c5)c34)nc(-c3cc(-c4ccccc4)c4c(c3)oc3ccccc34)n2)cc1 ; c1ccc(-c2nc(-c3ccc4c(c3)oc3cccc(-c5ccccc5-c5ccc6c(c5)oc5ccccc56)c34)nc(-c3cc(-c4ccccc4)c4c(c3)oc3ccccc34)n2)cc1]

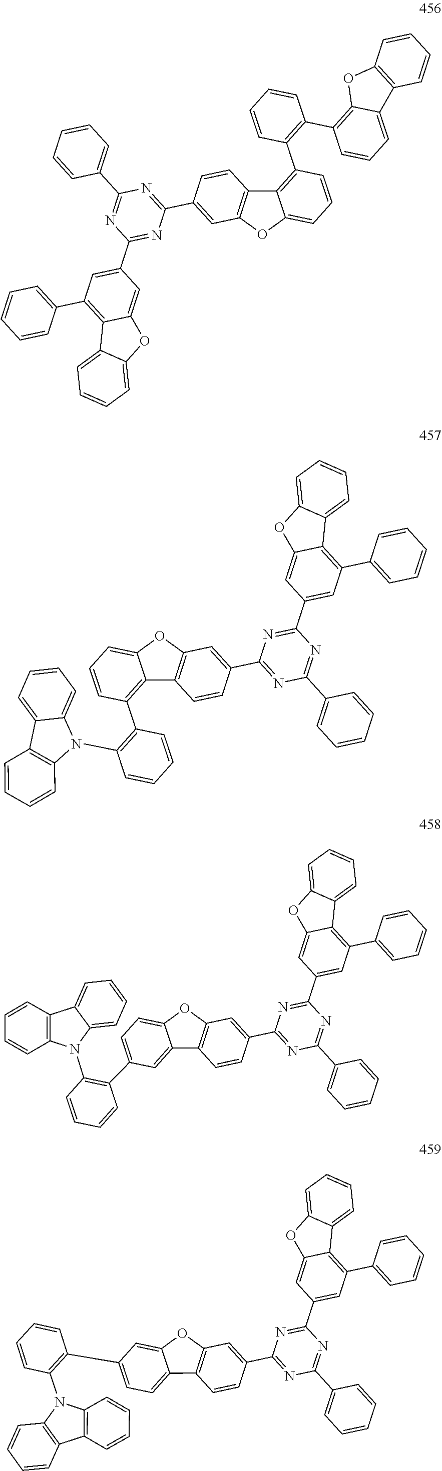 [SMILES: c1ccc(-c2nc(-c3ccc4c(c3)oc3cccc(-c5ccccc5-c5cccc6c5oc5ccccc56)c34)nc(-c3cc(-c4ccccc4)c4c(c3)oc3ccccc34)n2)cc1 ; c1ccc(-c2nc(-c3ccc4c(c3)oc3cccc(-c5ccccc5-n5c6ccccc6c6ccccc65)c34)nc(-c3cc(-c4ccccc4)c4c(c3)oc3ccccc34)n2)cc1 ; c1ccc(-c2nc(-c3ccc4c(c3)oc3ccc(-c5ccccc5-n5c6ccccc6c6ccccc65)cc34)nc(-c3cc(-c4ccccc4)c4c(c3)oc3ccccc34)n2)cc1 ; c1ccc(-c2nc(-c3ccc4c(c3)oc3cc(-c5ccccc5-n5c6ccccc6c6ccccc65)ccc34)nc(-c3cc(-c4ccccc4)c4c(c3)oc3ccccc34)n2)cc1]

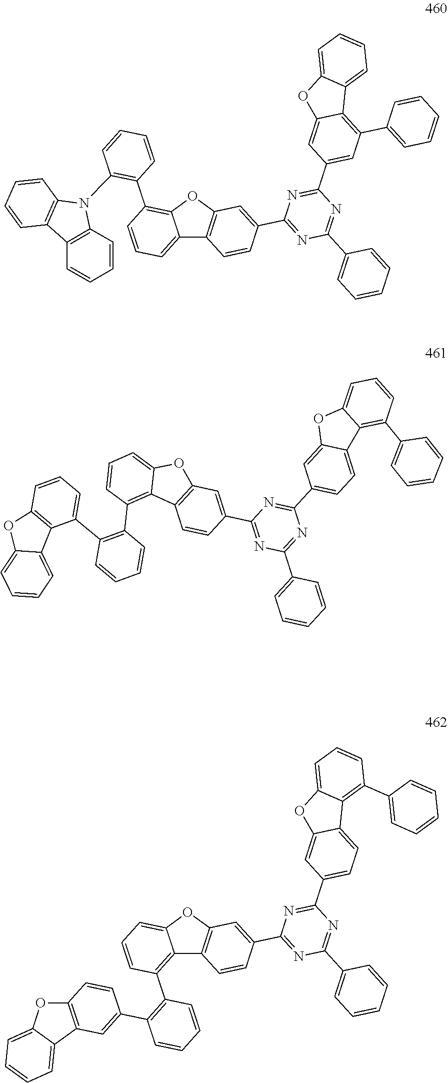 [SMILES: c1ccc(-c2nc(-c3ccc4c(c3)oc3c(-c5ccccc5-n5c6ccccc6c6ccccc65)cccc34)nc(-c3cc(-c4ccccc4)c4c(c3)oc3ccccc34)n2)cc1 ; c1ccc(-c2nc(-c3ccc4c(c3)oc3cccc(-c5ccccc5)c34)nc(-c3ccc4c(c3)oc3cccc(-c5ccccc5-c5cccc6oc7ccccc7c56)c34)n2)cc1 ; c1ccc(-c2nc(-c3ccc4c(c3)oc3cccc(-c5ccccc5)c34)nc(-c3ccc4c(c3)oc3cccc(-c5ccccc5-c5ccc6oc7ccccc7c6c5)c34)n2)cc1]

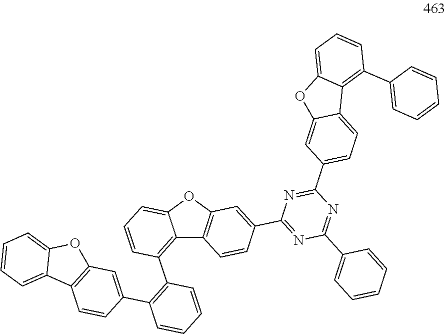 [SMILES: c1ccc(-c2nc(-c3ccc4c(c3)oc3cccc(-c5ccccc5)c34)nc(-c3ccc4c(c3)oc3cccc(-c5ccccc5-c5ccc6c(c5)oc5ccccc56)c34)n2)cc1]

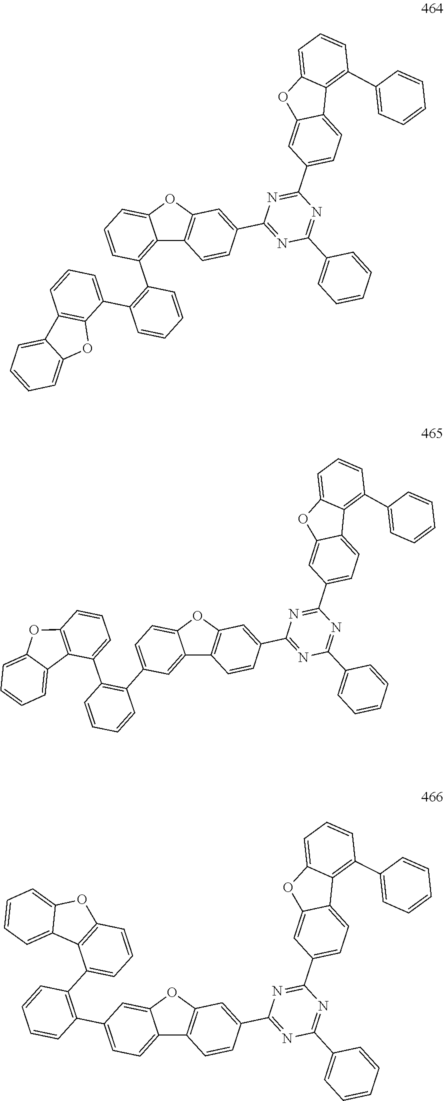 [SMILES: c1ccc(-c2nc(-c3ccc4c(c3)oc3cccc(-c5ccccc5)c34)nc(-c3ccc4c(c3)oc3cccc(-c5ccccc5-c5cccc6c5oc5ccccc56)c34)n2)cc1 ; c1ccc(-c2nc(-c3ccc4c(c3)oc3ccc(-c5ccccc5-c5cccc6oc7ccccc7c56)cc34)nc(-c3ccc4c(c3)oc3cccc(-c5ccccc5)c34)n2)cc1 ; c1ccc(-c2nc(-c3ccc4c(c3)oc3cc(-c5ccccc5-c5cccc6oc7ccccc7c56)ccc34)nc(-c3ccc4c(c3)oc3cccc(-c5ccccc5)c34)n2)cc1]

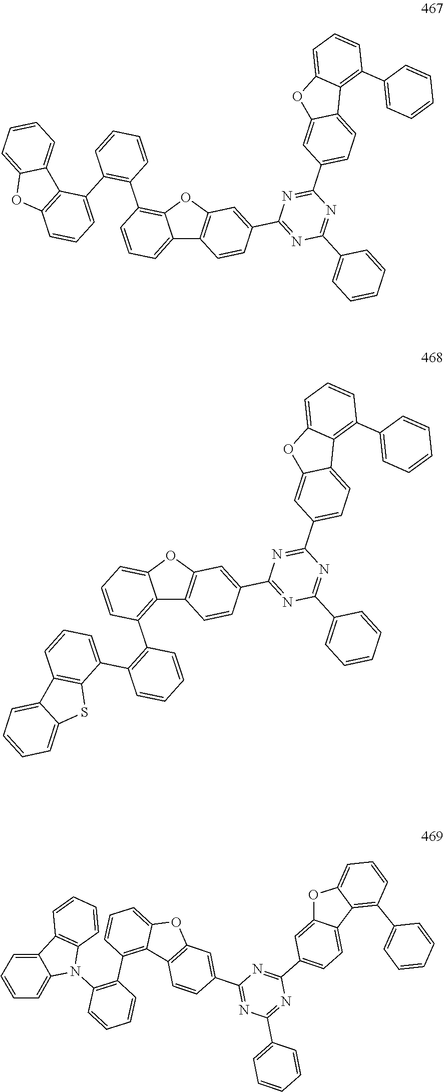 [SMILES: c1ccc(-c2nc(-c3ccc4c(c3)oc3c(-c5ccccc5-c5cccc6oc7ccccc7c56)cccc34)nc(-c3ccc4c(c3)oc3cccc(-c5ccccc5)c34)n2)cc1 ; c1ccc(-c2nc(-c3ccc4c(c3)oc3cccc(-c5ccccc5)c34)nc(-c3ccc4c(c3)oc3cccc(-c5ccccc5-c5cccc6c5sc5ccccc56)c34)n2)cc1 ; c1ccc(-c2nc(-c3ccc4c(c3)oc3cccc(-c5ccccc5)c34)nc(-c3ccc4c(c3)oc3cccc(-c5ccccc5-n5c6ccccc6c6ccccc65)c34)n2)cc1]

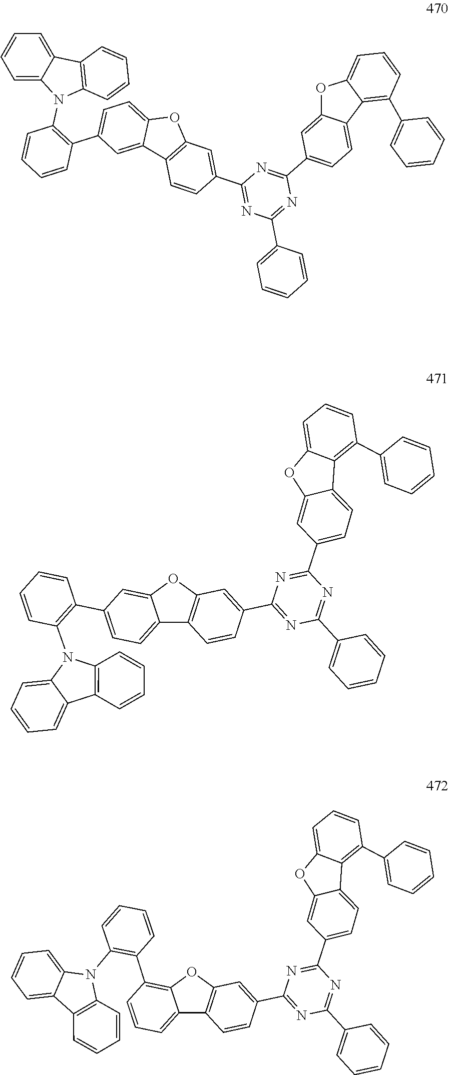 [SMILES: c1ccc(-c2nc(-c3ccc4c(c3)oc3ccc(-c5ccccc5-n5c6ccccc6c6ccccc65)cc34)nc(-c3ccc4c(c3)oc3cccc(-c5ccccc5)c34)n2)cc1 ; c1ccc(-c2nc(-c3ccc4c(c3)oc3cc(-c5ccccc5-n5c6ccccc6c6ccccc65)ccc34)nc(-c3ccc4c(c3)oc3cccc(-c5ccccc5)c34)n2)cc1 ; c1ccc(-c2nc(-c3ccc4c(c3)oc3c(-c5ccccc5-n5c6ccccc6c6ccccc65)cccc34)nc(-c3ccc4c(c3)oc3cccc(-c5ccccc5)c34)n2)cc1]

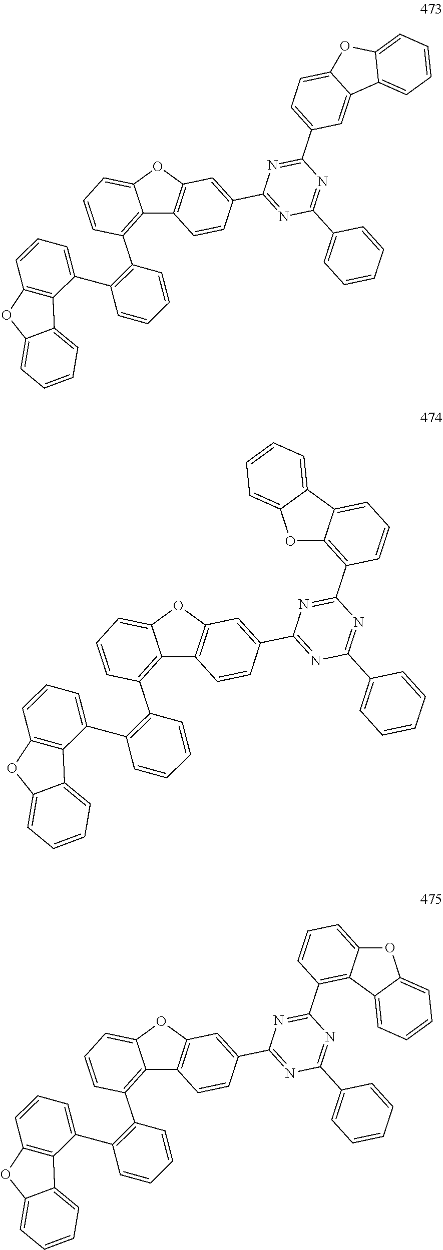 [SMILES: c1ccc(-c2nc(-c3ccc4c(c3)oc3cccc(-c5ccccc5-c5cccc6oc7ccccc7c56)c34)nc(-c3ccc4oc5ccccc5c4c3)n2)cc1 ; c1ccc(-c2nc(-c3ccc4c(c3)oc3cccc(-c5ccccc5-c5cccc6oc7ccccc7c56)c34)nc(-c3cccc4c3oc3ccccc34)n2)cc1 ; c1ccc(-c2nc(-c3ccc4c(c3)oc3cccc(-c5ccccc5-c5cccc6oc7ccccc7c56)c34)nc(-c3cccc4oc5ccccc5c34)n2)cc1]

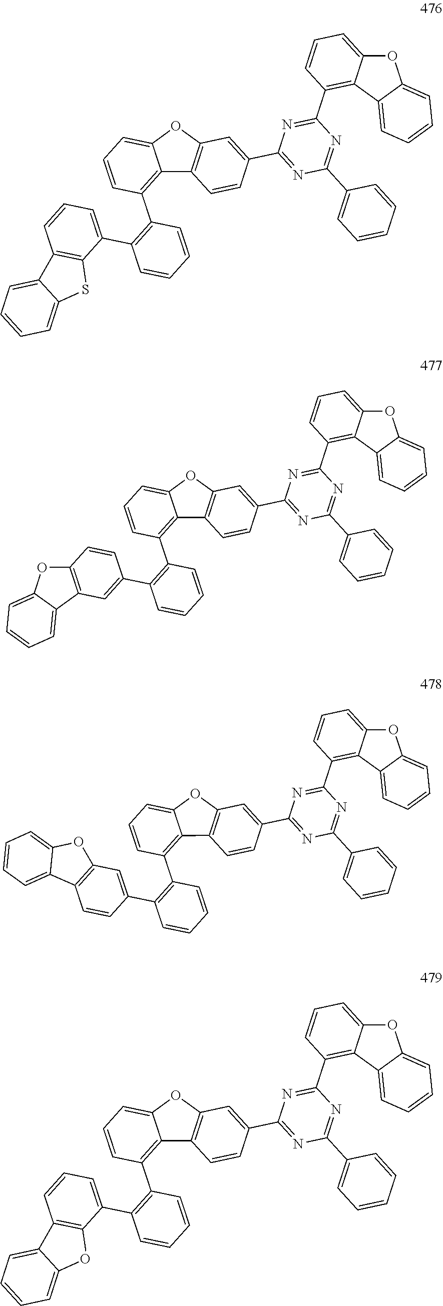 [SMILES: c1ccc(-c2nc(-c3ccc4c(c3)oc3cccc(-c5ccccc5-c5cccc6c5sc5ccccc56)c34)nc(-c3cccc4oc5ccccc5c34)n2)cc1 ; c1ccc(-c2nc(-c3ccc4c(c3)oc3cccc(-c5ccccc5-c5ccc6oc7ccccc7c6c5)c34)nc(-c3cccc4oc5ccccc5c34)n2)cc1 ; c1ccc(-c2nc(-c3ccc4c(c3)oc3cccc(-c5ccccc5-c5ccc6c(c5)oc5ccccc56)c34)nc(-c3cccc4oc5ccccc5c34)n2)cc1 ; c1ccc(-c2nc(-c3ccc4c(c3)oc3cccc(-c5ccccc5-c5cccc6c5oc5ccccc56)c34)nc(-c3cccc4oc5ccccc5c34)n2)cc1]

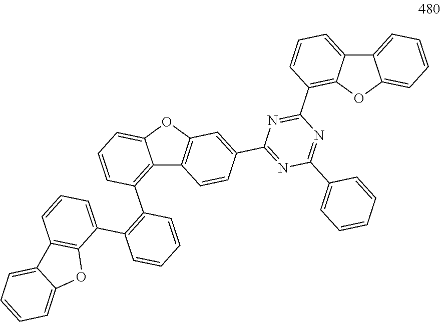 [SMILES: c1ccc(-c2nc(-c3ccc4c(c3)oc3cccc(-c5ccccc5-c5cccc6c5oc5ccccc56)c34)nc(-c3cccc4c3oc3ccccc34)n2)cc1]

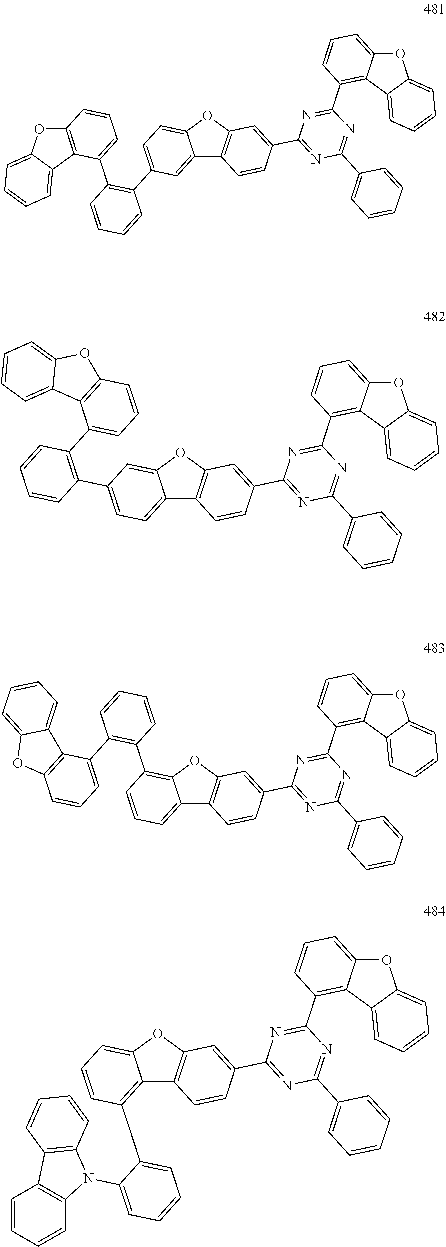 [SMILES: c1ccc(-c2nc(-c3ccc4c(c3)oc3ccc(-c5ccccc5-c5cccc6oc7ccccc7c56)cc34)nc(-c3cccc4oc5ccccc5c34)n2)cc1 ; c1ccc(-c2nc(-c3ccc4c(c3)oc3cc(-c5ccccc5-c5cccc6oc7ccccc7c56)ccc34)nc(-c3cccc4oc5ccccc5c34)n2)cc1 ; c1ccc(-c2nc(-c3ccc4c(c3)oc3c(-c5ccccc5-c5cccc6oc7ccccc7c56)cccc34)nc(-c3cccc4oc5ccccc5c34)n2)cc1 ; c1ccc(-c2nc(-c3ccc4c(c3)oc3cccc(-c5ccccc5-n5c6ccccc6c6ccccc65)c34)nc(-c3cccc4oc5ccccc5c34)n2)cc1]

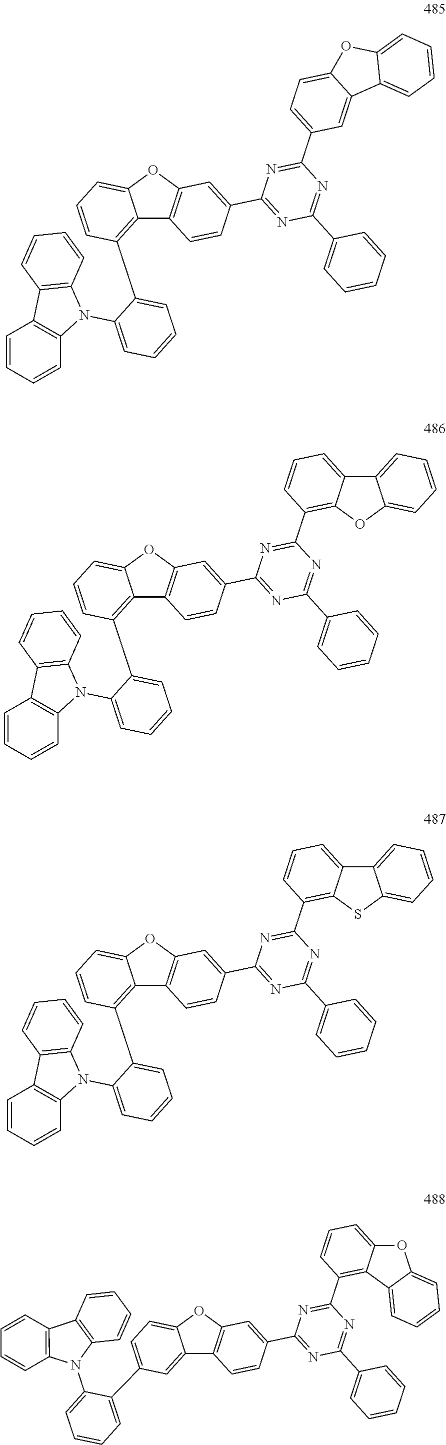 [SMILES: c1ccc(-c2nc(-c3ccc4c(c3)oc3cccc(-c5ccccc5-n5c6ccccc6c6ccccc65)c34)nc(-c3ccc4oc5ccccc5c4c3)n2)cc1 ; c1ccc(-c2nc(-c3ccc4c(c3)oc3cccc(-c5ccccc5-n5c6ccccc6c6ccccc65)c34)nc(-c3cccc4c3oc3ccccc34)n2)cc1 ; c1ccc(-c2nc(-c3ccc4c(c3)oc3cccc(-c5ccccc5-n5c6ccccc6c6ccccc65)c34)nc(-c3cccc4c3sc3ccccc34)n2)cc1 ; c1ccc(-c2nc(-c3ccc4c(c3)oc3ccc(-c5ccccc5-n5c6ccccc6c6ccccc65)cc34)nc(-c3cccc4oc5ccccc5c34)n2)cc1]

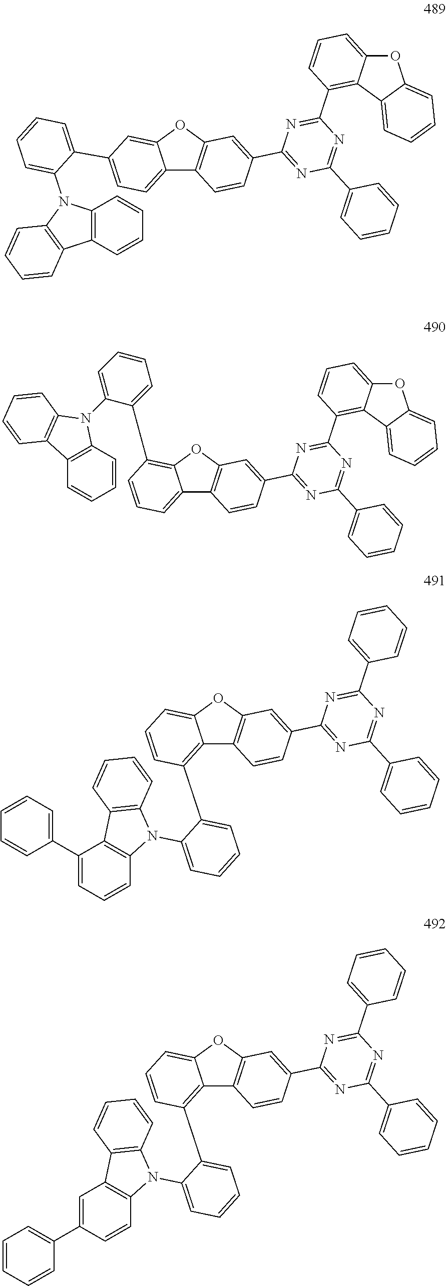 [SMILES: c1ccc(-c2nc(-c3ccc4c(c3)oc3cc(-c5ccccc5-n5c6ccccc6c6ccccc65)ccc34)nc(-c3cccc4oc5ccccc5c34)n2)cc1 ; c1ccc(-c2nc(-c3ccc4c(c3)oc3c(-c5ccccc5-n5c6ccccc6c6ccccc65)cccc34)nc(-c3cccc4oc5ccccc5c34)n2)cc1 ; c1ccc(-c2nc(-c3ccccc3)nc(-c3ccc4c(c3)oc3cccc(-c5ccccc5-n5c6ccccc6c6c(-c7ccccc7)cccc65)c34)n2)cc1 ; c1ccc(-c2ccc3c(c2)c2ccccc2n3-c2ccccc2-c2cccc3oc4cc(-c5nc(-c6ccccc6)nc(-c6ccccc6)n5)ccc4c23)cc1]

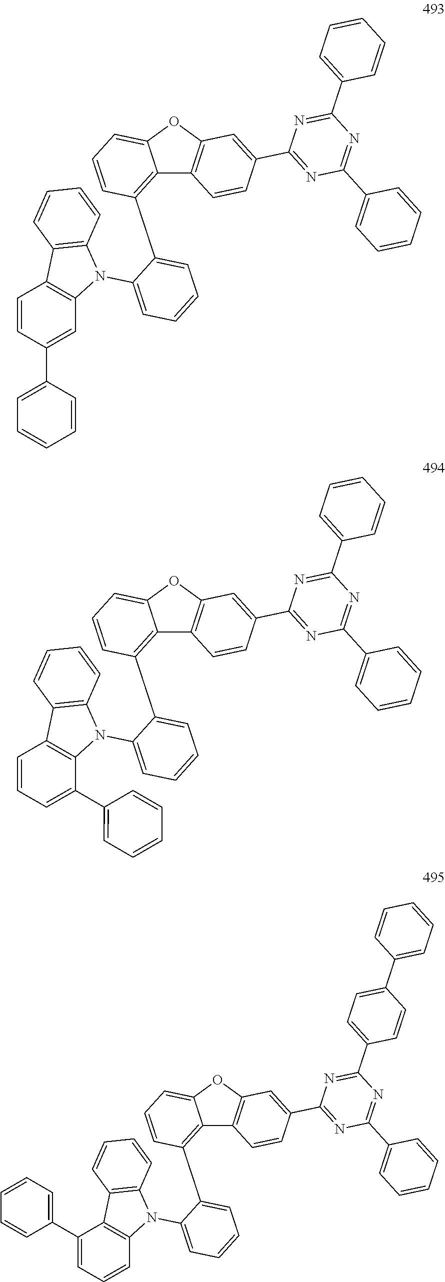 [SMILES: c1ccc(-c2ccc3c4ccccc4n(-c4ccccc4-c4cccc5oc6cc(-c7nc(-c8ccccc8)nc(-c8ccccc8)n7)ccc6c45)c3c2)cc1 ; c1ccc(-c2nc(-c3ccccc3)nc(-c3ccc4c(c3)oc3cccc(-c5ccccc5-n5c6ccccc6c6cccc(-c7ccccc7)c65)c34)n2)cc1 ; c1ccc(-c2ccc(-c3nc(-c4ccccc4)nc(-c4ccc5c(c4)oc4cccc(-c6ccccc6-n6c7ccccc7c7c(-c8ccccc8)cccc76)c45)n3)cc2)cc1]

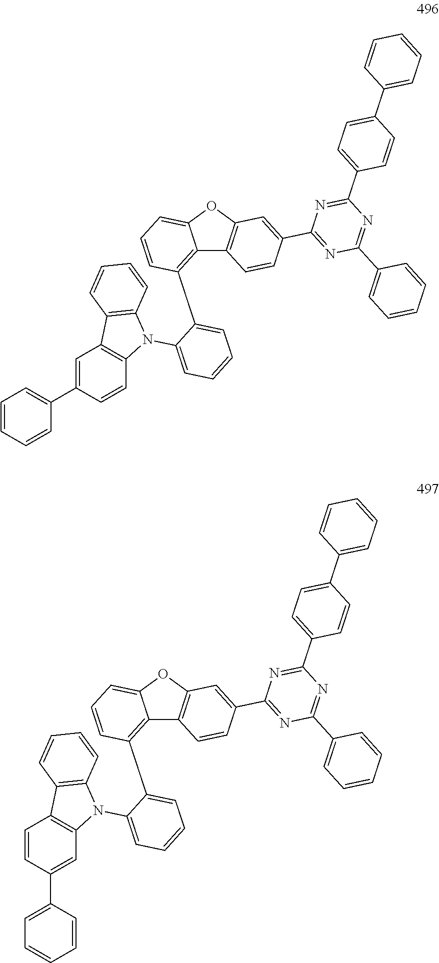 [SMILES: c1ccc(-c2ccc(-c3nc(-c4ccccc4)nc(-c4ccc5c(c4)oc4cccc(-c6ccccc6-n6c7ccccc7c7cc(-c8ccccc8)ccc76)c45)n3)cc2)cc1 ; c1ccc(-c2ccc(-c3nc(-c4ccccc4)nc(-c4ccc5c(c4)oc4cccc(-c6ccccc6-n6c7ccccc7c7ccc(-c8ccccc8)cc76)c45)n3)cc2)cc1]

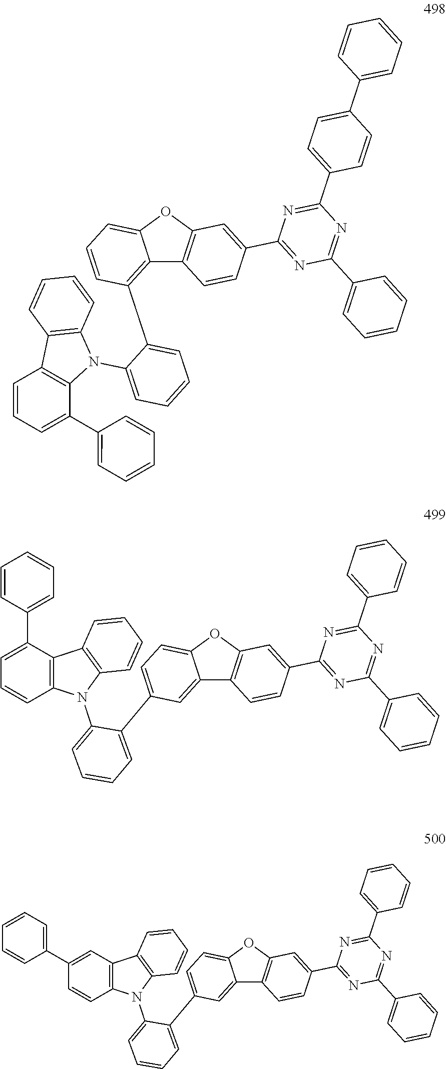 [SMILES: c1ccc(-c2ccc(-c3nc(-c4ccccc4)nc(-c4ccc5c(c4)oc4cccc(-c6ccccc6-n6c7ccccc7c7cccc(-c8ccccc8)c76)c45)n3)cc2)cc1 ; c1ccc(-c2nc(-c3ccccc3)nc(-c3ccc4c(c3)oc3ccc(-c5ccccc5-n5c6ccccc6c6c(-c7ccccc7)cccc65)cc34)n2)cc1 ; c1ccc(-c2ccc3c(c2)c2ccccc2n3-c2ccccc2-c2ccc3oc4cc(-c5nc(-c6ccccc6)nc(-c6ccccc6)n5)ccc4c3c2)cc1]

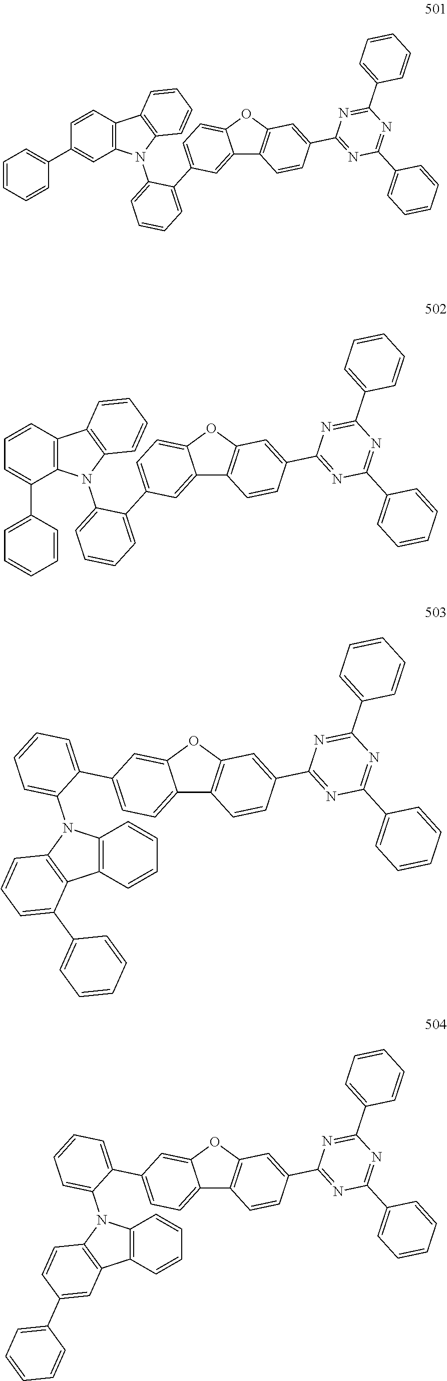 [SMILES: c1ccc(-c2ccc3c4ccccc4n(-c4ccccc4-c4ccc5oc6cc(-c7nc(-c8ccccc8)nc(-c8ccccc8)n7)ccc6c5c4)c3c2)cc1 ; c1ccc(-c2nc(-c3ccccc3)nc(-c3ccc4c(c3)oc3ccc(-c5ccccc5-n5c6ccccc6c6cccc(-c7ccccc7)c65)cc34)n2)cc1 ; c1ccc(-c2nc(-c3ccccc3)nc(-c3ccc4c(c3)oc3cc(-c5ccccc5-n5c6ccccc6c6c(-c7ccccc7)cccc65)ccc34)n2)cc1 ; c1ccc(-c2ccc3c(c2)c2ccccc2n3-c2ccccc2-c2ccc3c(c2)oc2cc(-c4nc(-c5ccccc5)nc(-c5ccccc5)n4)ccc23)cc1]

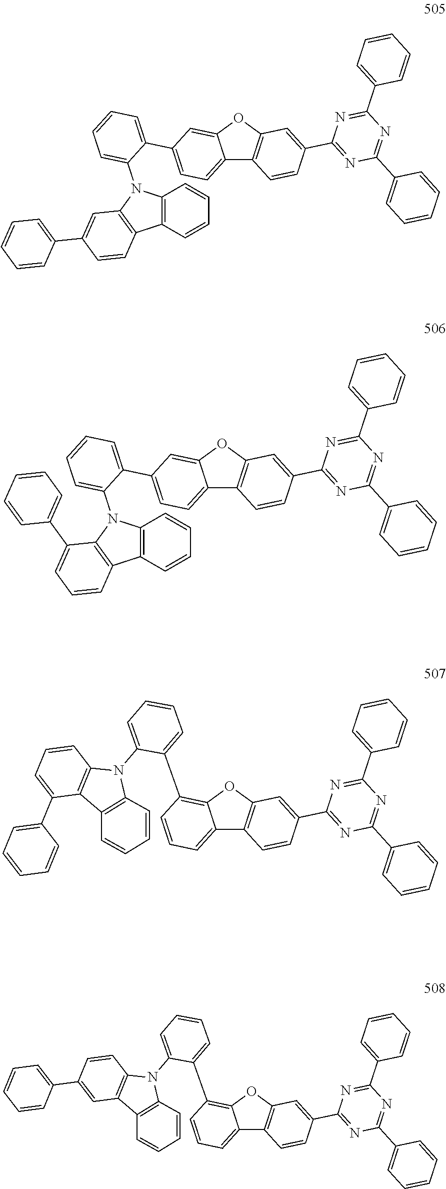 [SMILES: c1ccc(-c2ccc3c4ccccc4n(-c4ccccc4-c4ccc5c(c4)oc4cc(-c6nc(-c7ccccc7)nc(-c7ccccc7)n6)ccc45)c3c2)cc1 ; c1ccc(-c2nc(-c3ccccc3)nc(-c3ccc4c(c3)oc3cc(-c5ccccc5-n5c6ccccc6c6cccc(-c7ccccc7)c65)ccc34)n2)cc1 ; c1ccc(-c2nc(-c3ccccc3)nc(-c3ccc4c(c3)oc3c(-c5ccccc5-n5c6ccccc6c6c(-c7ccccc7)cccc65)cccc34)n2)cc1 ; c1ccc(-c2ccc3c(c2)c2ccccc2n3-c2ccccc2-c2cccc3c2oc2cc(-c4nc(-c5ccccc5)nc(-c5ccccc5)n4)ccc23)cc1]

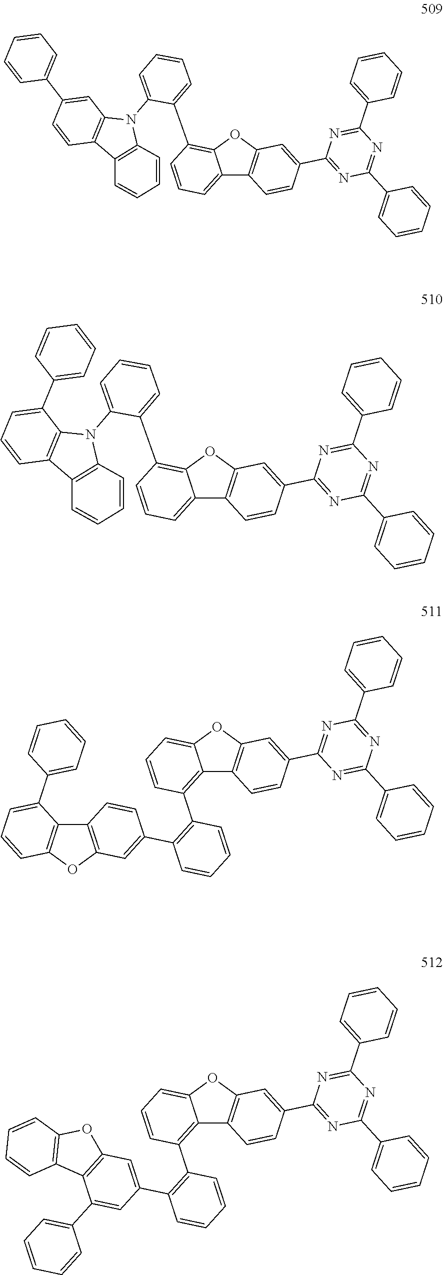 [SMILES: c1ccc(-c2ccc3c4ccccc4n(-c4ccccc4-c4cccc5c4oc4cc(-c6nc(-c7ccccc7)nc(-c7ccccc7)n6)ccc45)c3c2)cc1 ; c1ccc(-c2nc(-c3ccccc3)nc(-c3ccc4c(c3)oc3c(-c5ccccc5-n5c6ccccc6c6cccc(-c7ccccc7)c65)cccc34)n2)cc1 ; c1ccc(-c2nc(-c3ccccc3)nc(-c3ccc4c(c3)oc3cccc(-c5ccccc5-c5ccc6c(c5)oc5cccc(-c7ccccc7)c56)c34)n2)cc1 ; c1ccc(-c2nc(-c3ccccc3)nc(-c3ccc4c(c3)oc3cccc(-c5ccccc5-c5cc(-c6ccccc6)c6c(c5)oc5ccccc56)c34)n2)cc1]

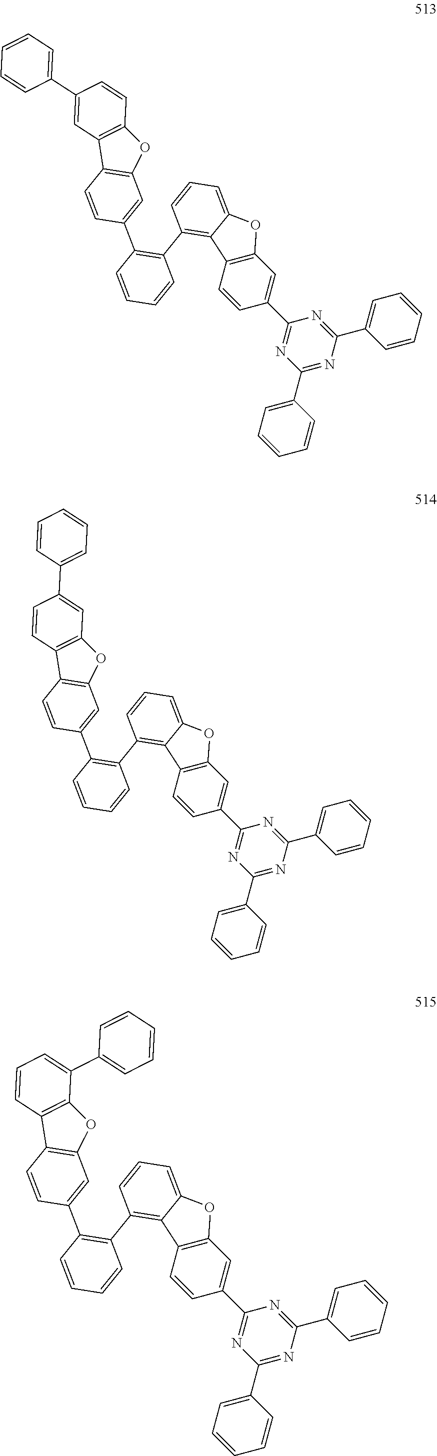 [SMILES: c1ccc(-c2ccc3oc4cc(-c5ccccc5-c5cccc6oc7cc(-c8nc(-c9ccccc9)nc(-c9ccccc9)n8)ccc7c56)ccc4c3c2)cc1 ; c1ccc(-c2ccc3c(c2)oc2cc(-c4ccccc4-c4cccc5oc6cc(-c7nc(-c8ccccc8)nc(-c8ccccc8)n7)ccc6c45)ccc23)cc1 ; c1ccc(-c2nc(-c3ccccc3)nc(-c3ccc4c(c3)oc3cccc(-c5ccccc5-c5ccc6c(c5)oc5c(-c7ccccc7)cccc56)c34)n2)cc1]

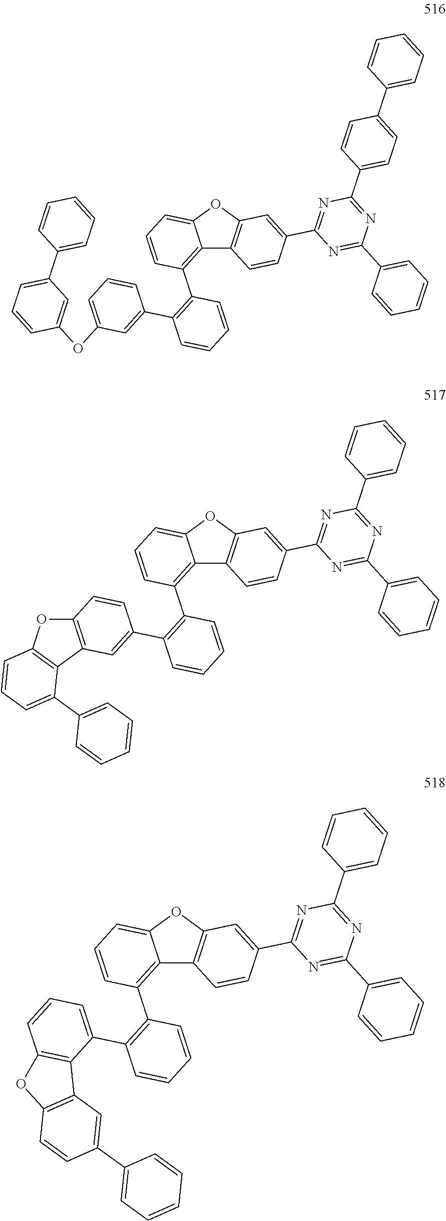 [SMILES: c1ccc(-c2ccc(-c3nc(-c4ccccc4)nc(-c4ccc5c(c4)oc4cccc(-c6ccccc6-c6cccc(Oc7cccc(-c8ccccc8)c7)c6)c45)n3)cc2)cc1 ; c1ccc(-c2nc(-c3ccccc3)nc(-c3ccc4c(c3)oc3cccc(-c5ccccc5-c5ccc6oc7cccc(-c8ccccc8)c7c6c5)c34)n2)cc1 ; c1ccc(-c2ccc3oc4cccc(-c5ccccc5-c5cccc6oc7cc(-c8nc(-c9ccccc9)nc(-c9ccccc9)n8)ccc7c56)c4c3c2)cc1]

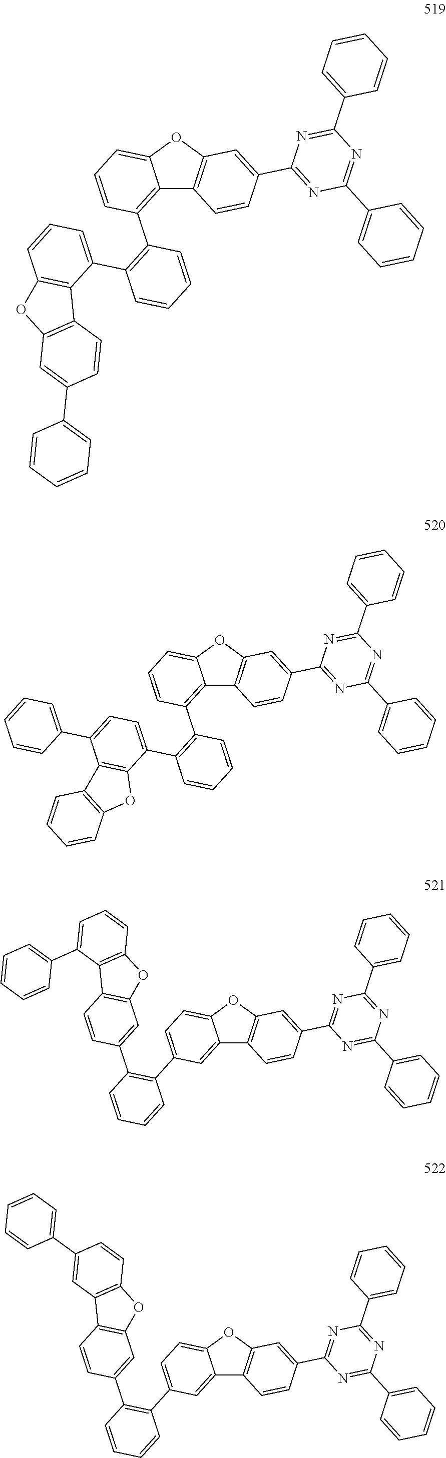 [SMILES: c1ccc(-c2ccc3c(c2)oc2cccc(-c4ccccc4-c4cccc5oc6cc(-c7nc(-c8ccccc8)nc(-c8ccccc8)n7)ccc6c45)c23)cc1 ; c1ccc(-c2nc(-c3ccccc3)nc(-c3ccc4c(c3)oc3cccc(-c5ccccc5-c5ccc(-c6ccccc6)c6c5oc5ccccc56)c34)n2)cc1 ; c1ccc(-c2nc(-c3ccccc3)nc(-c3ccc4c(c3)oc3ccc(-c5ccccc5-c5ccc6c(c5)oc5cccc(-c7ccccc7)c56)cc34)n2)cc1 ; c1ccc(-c2ccc3oc4cc(-c5ccccc5-c5ccc6oc7cc(-c8nc(-c9ccccc9)nc(-c9ccccc9)n8)ccc7c6c5)ccc4c3c2)cc1]

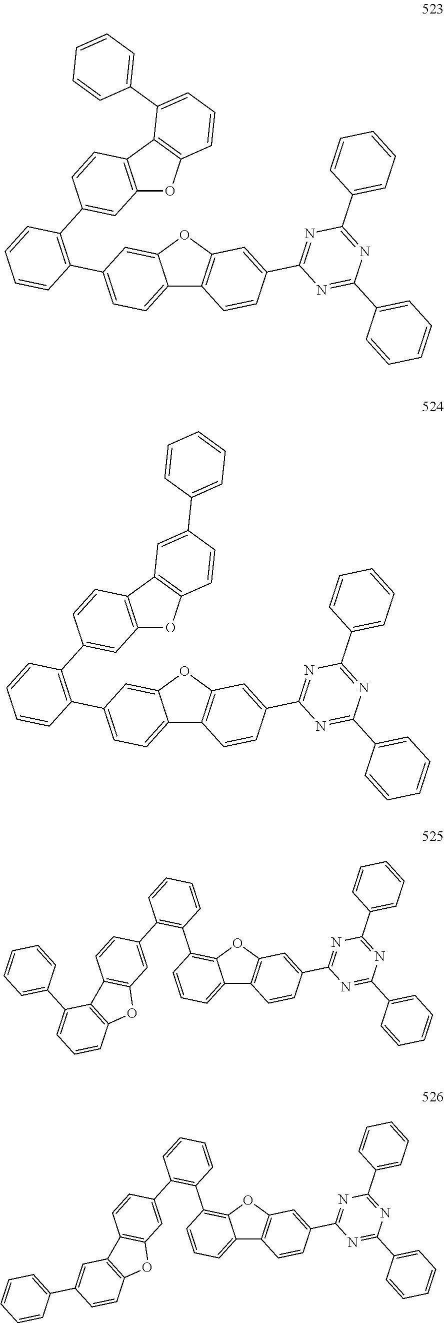 [SMILES: c1ccc(-c2nc(-c3ccccc3)nc(-c3ccc4c(c3)oc3cc(-c5ccccc5-c5ccc6c(c5)oc5cccc(-c7ccccc7)c56)ccc34)n2)cc1 ; c1ccc(-c2ccc3oc4cc(-c5ccccc5-c5ccc6c(c5)oc5cc(-c7nc(-c8ccccc8)nc(-c8ccccc8)n7)ccc56)ccc4c3c2)cc1 ; c1ccc(-c2nc(-c3ccccc3)nc(-c3ccc4c(c3)oc3c(-c5ccccc5-c5ccc6c(c5)oc5cccc(-c7ccccc7)c56)cccc34)n2)cc1 ; c1ccc(-c2ccc3oc4cc(-c5ccccc5-c5cccc6c5oc5cc(-c7nc(-c8ccccc8)nc(-c8ccccc8)n7)ccc56)ccc4c3c2)cc1]

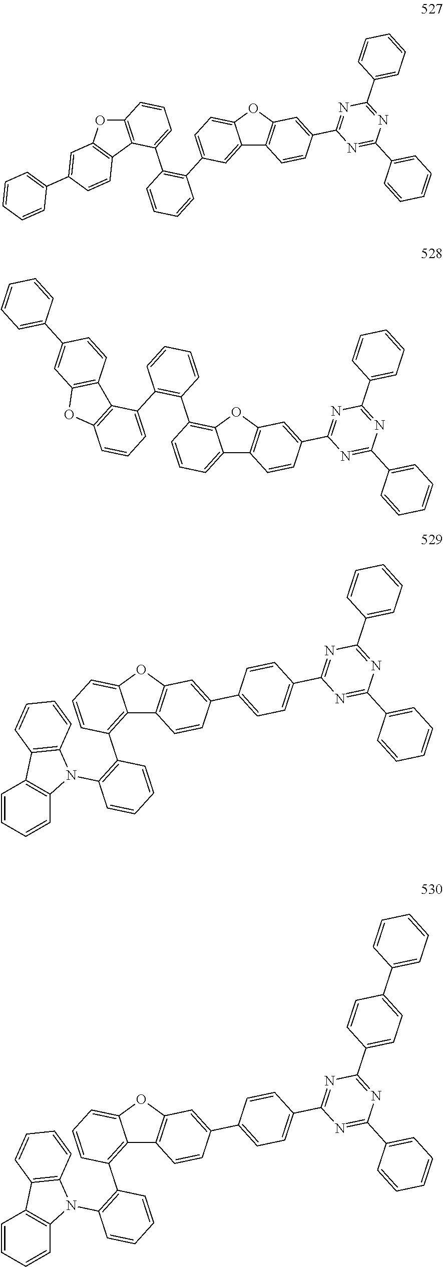 [SMILES: c1ccc(-c2ccc3c(c2)oc2cccc(-c4ccccc4-c4ccc5oc6cc(-c7nc(-c8ccccc8)nc(-c8ccccc8)n7)ccc6c5c4)c23)cc1 ; c1ccc(-c2ccc3c(c2)oc2cccc(-c4ccccc4-c4cccc5c4oc4cc(-c6nc(-c7ccccc7)nc(-c7ccccc7)n6)ccc45)c23)cc1 ; c1ccc(-c2nc(-c3ccccc3)nc(-c3ccc(-c4ccc5c(c4)oc4cccc(-c6ccccc6-n6c7ccccc7c7ccccc76)c45)cc3)n2)cc1 ; c1ccc(-c2ccc(-c3nc(-c4ccccc4)nc(-c4ccc(-c5ccc6c(c5)oc5cccc(-c7ccccc7-n7c8ccccc8c8ccccc87)c56)cc4)n3)cc2)cc1]

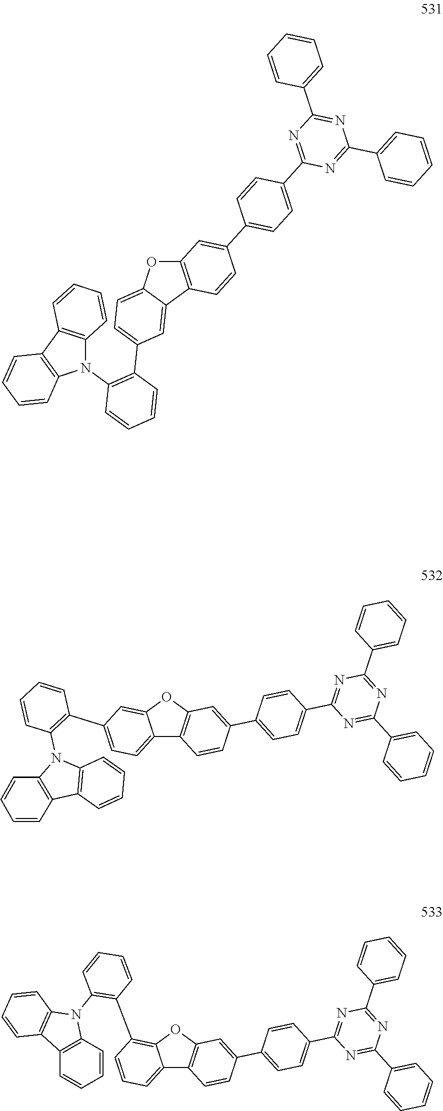 [SMILES: c1ccc(-c2nc(-c3ccccc3)nc(-c3ccc(-c4ccc5c(c4)oc4ccc(-c6ccccc6-n6c7ccccc7c7ccccc76)cc45)cc3)n2)cc1 ; c1ccc(-c2nc(-c3ccccc3)nc(-c3ccc(-c4ccc5c(c4)oc4cc(-c6ccccc6-n6c7ccccc7c7ccccc76)ccc45)cc3)n2)cc1 ; c1ccc(-c2nc(-c3ccccc3)nc(-c3ccc(-c4ccc5c(c4)oc4c(-c6ccccc6-n6c7ccccc7c7ccccc76)cccc45)cc3)n2)cc1]

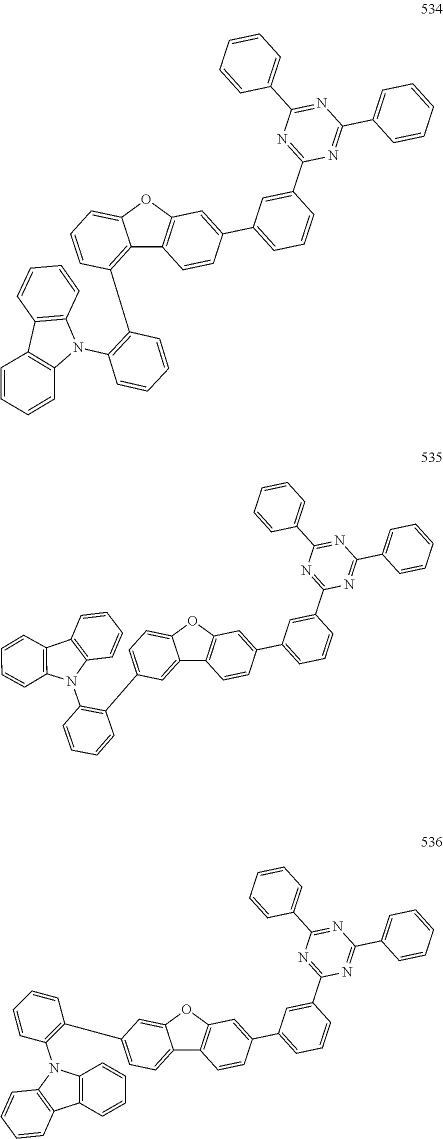 [SMILES: c1ccc(-c2nc(-c3ccccc3)nc(-c3cccc(-c4ccc5c(c4)oc4cccc(-c6ccccc6-n6c7ccccc7c7ccccc76)c45)c3)n2)cc1 ; c1ccc(-c2nc(-c3ccccc3)nc(-c3cccc(-c4ccc5c(c4)oc4ccc(-c6ccccc6-n6c7ccccc7c7ccccc76)cc45)c3)n2)cc1 ; c1ccc(-c2nc(-c3ccccc3)nc(-c3cccc(-c4ccc5c(c4)oc4cc(-c6ccccc6-n6c7ccccc7c7ccccc76)ccc45)c3)n2)cc1]

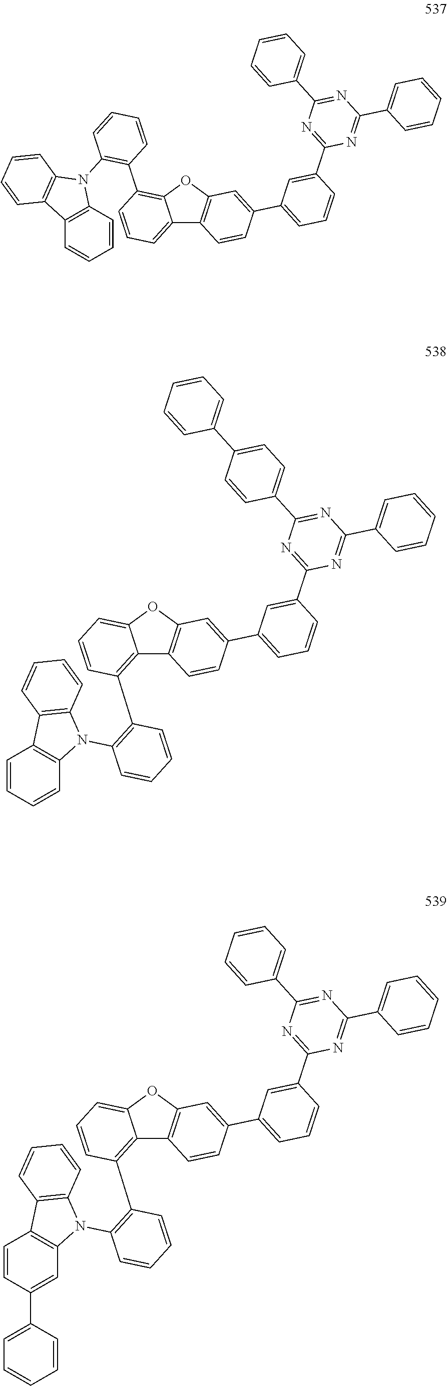 [SMILES: c1ccc(-c2nc(-c3ccccc3)nc(-c3cccc(-c4ccc5c(c4)oc4c(-c6ccccc6-n6c7ccccc7c7ccccc76)cccc45)c3)n2)cc1 ; c1ccc(-c2ccc(-c3nc(-c4ccccc4)nc(-c4cccc(-c5ccc6c(c5)oc5cccc(-c7ccccc7-n7c8ccccc8c8ccccc87)c56)c4)n3)cc2)cc1 ; c1ccc(-c2ccc3c4ccccc4n(-c4ccccc4-c4cccc5oc6cc(-c7cccc(-c8nc(-c9ccccc9)nc(-c9ccccc9)n8)c7)ccc6c45)c3c2)cc1]

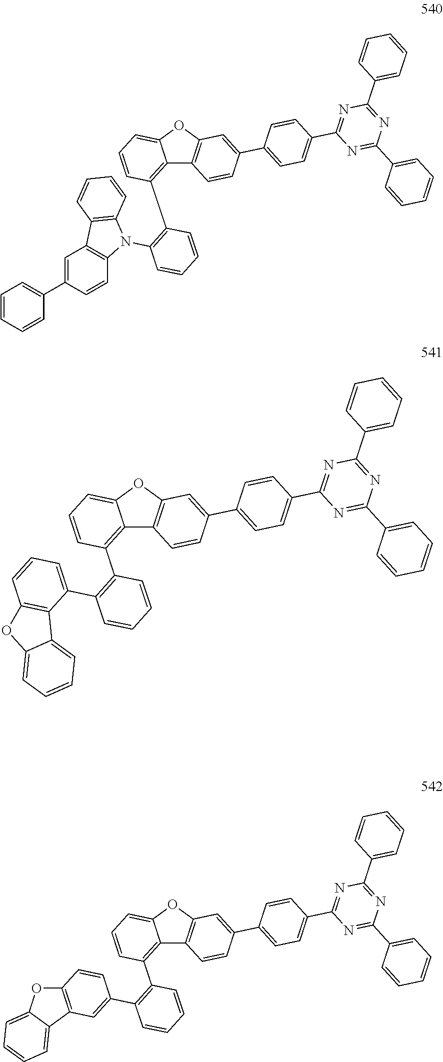 [SMILES: c1ccc(-c2ccc3c(c2)c2ccccc2n3-c2ccccc2-c2cccc3oc4cc(-c5ccc(-c6nc(-c7ccccc7)nc(-c7ccccc7)n6)cc5)ccc4c23)cc1 ; c1ccc(-c2nc(-c3ccccc3)nc(-c3ccc(-c4ccc5c(c4)oc4cccc(-c6ccccc6-c6cccc7oc8ccccc8c67)c45)cc3)n2)cc1 ; c1ccc(-c2nc(-c3ccccc3)nc(-c3ccc(-c4ccc5c(c4)oc4cccc(-c6ccccc6-c6ccc7oc8ccccc8c7c6)c45)cc3)n2)cc1]

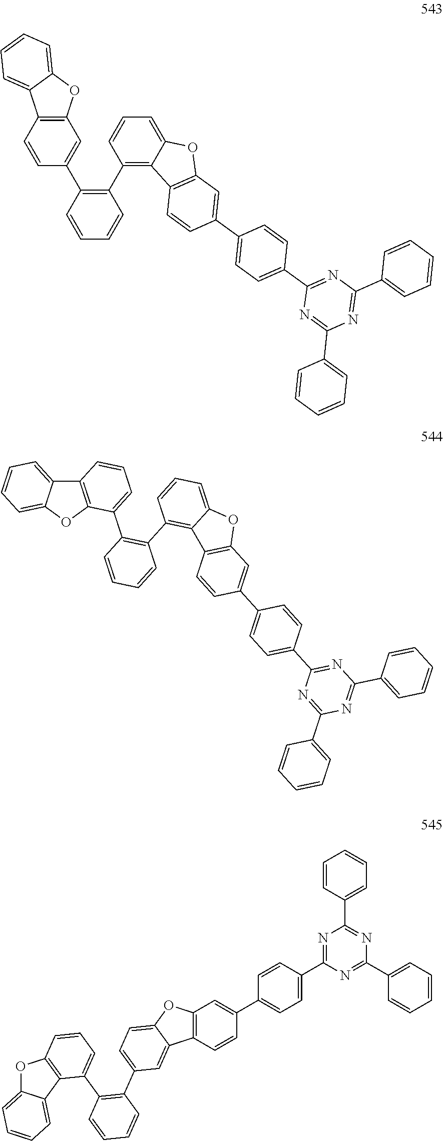 [SMILES: c1ccc(-c2nc(-c3ccccc3)nc(-c3ccc(-c4ccc5c(c4)oc4cccc(-c6ccccc6-c6ccc7c(c6)oc6ccccc67)c45)cc3)n2)cc1 ; c1ccc(-c2nc(-c3ccccc3)nc(-c3ccc(-c4ccc5c(c4)oc4cccc(-c6ccccc6-c6cccc7c6oc6ccccc67)c45)cc3)n2)cc1 ; c1ccc(-c2nc(-c3ccccc3)nc(-c3ccc(-c4ccc5c(c4)oc4ccc(-c6ccccc6-c6cccc7oc8ccccc8c67)cc45)cc3)n2)cc1]

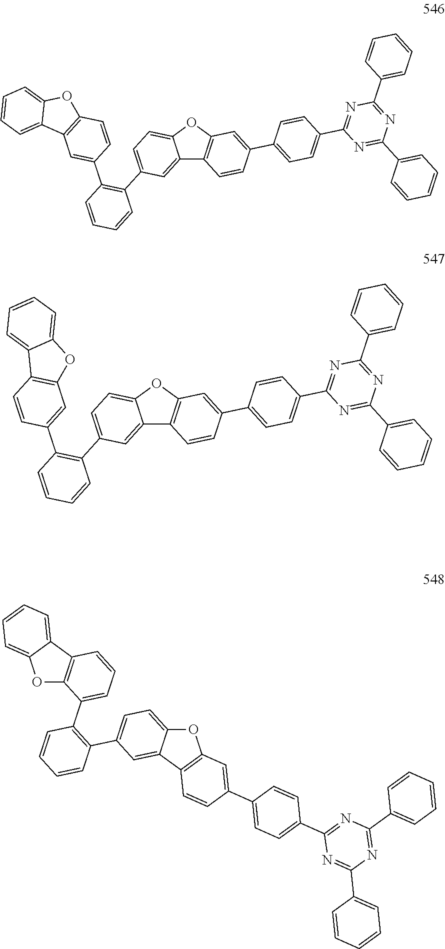 [SMILES: c1ccc(-c2nc(-c3ccccc3)nc(-c3ccc(-c4ccc5c(c4)oc4ccc(-c6ccccc6-c6ccc7oc8ccccc8c7c6)cc45)cc3)n2)cc1 ; c1ccc(-c2nc(-c3ccccc3)nc(-c3ccc(-c4ccc5c(c4)oc4ccc(-c6ccccc6-c6ccc7c(c6)oc6ccccc67)cc45)cc3)n2)cc1 ; c1ccc(-c2nc(-c3ccccc3)nc(-c3ccc(-c4ccc5c(c4)oc4ccc(-c6ccccc6-c6cccc7c6oc6ccccc67)cc45)cc3)n2)cc1]

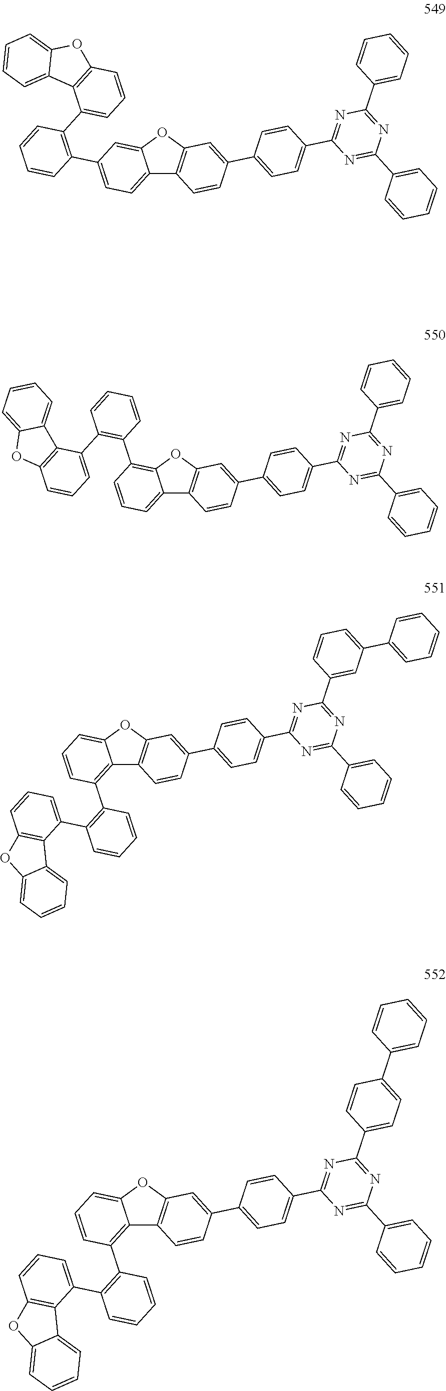 [SMILES: c1ccc(-c2nc(-c3ccccc3)nc(-c3ccc(-c4ccc5c(c4)oc4cc(-c6ccccc6-c6cccc7oc8ccccc8c67)ccc45)cc3)n2)cc1 ; c1ccc(-c2nc(-c3ccccc3)nc(-c3ccc(-c4ccc5c(c4)oc4c(-c6ccccc6-c6cccc7oc8ccccc8c67)cccc45)cc3)n2)cc1 ; c1ccc(-c2cccc(-c3nc(-c4ccccc4)nc(-c4ccc(-c5ccc6c(c5)oc5cccc(-c7ccccc7-c7cccc8oc9ccccc9c78)c56)cc4)n3)c2)cc1 ; c1ccc(-c2ccc(-c3nc(-c4ccccc4)nc(-c4ccc(-c5ccc6c(c5)oc5cccc(-c7ccccc7-c7cccc8oc9ccccc9c78)c56)cc4)n3)cc2)cc1]

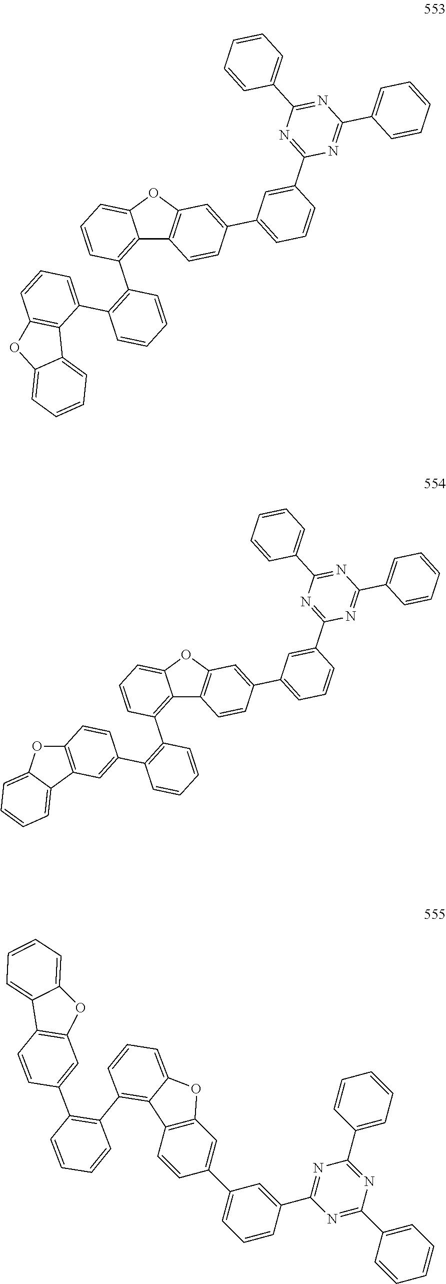 [SMILES: c1ccc(-c2nc(-c3ccccc3)nc(-c3cccc(-c4ccc5c(c4)oc4cccc(-c6ccccc6-c6cccc7oc8ccccc8c67)c45)c3)n2)cc1 ; c1ccc(-c2nc(-c3ccccc3)nc(-c3cccc(-c4ccc5c(c4)oc4cccc(-c6ccccc6-c6ccc7oc8ccccc8c7c6)c45)c3)n2)cc1 ; c1ccc(-c2nc(-c3ccccc3)nc(-c3cccc(-c4ccc5c(c4)oc4cccc(-c6ccccc6-c6ccc7c(c6)oc6ccccc67)c45)c3)n2)cc1]

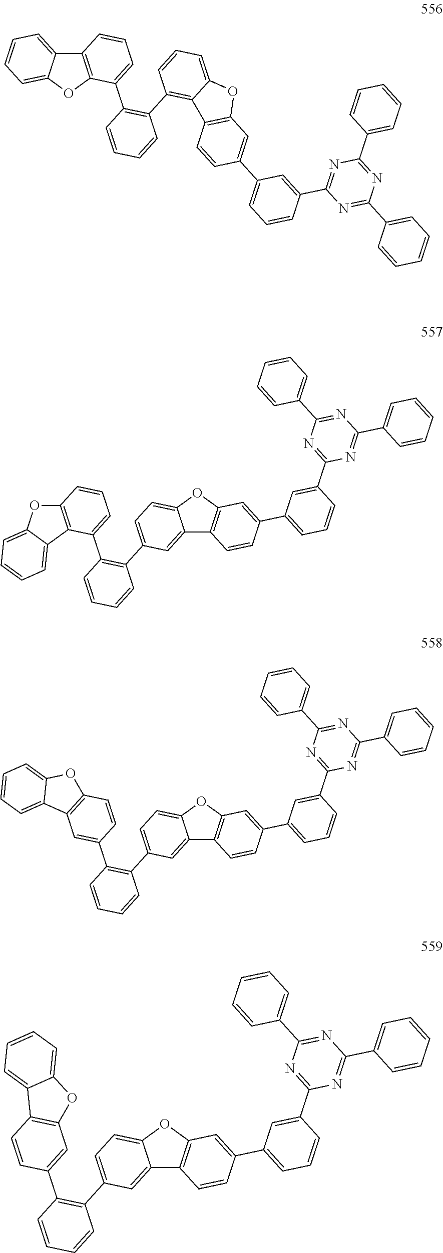 [SMILES: c1ccc(-c2nc(-c3ccccc3)nc(-c3cccc(-c4ccc5c(c4)oc4cccc(-c6ccccc6-c6cccc7c6oc6ccccc67)c45)c3)n2)cc1 ; c1ccc(-c2nc(-c3ccccc3)nc(-c3cccc(-c4ccc5c(c4)oc4ccc(-c6ccccc6-c6cccc7oc8ccccc8c67)cc45)c3)n2)cc1 ; c1ccc(-c2nc(-c3ccccc3)nc(-c3cccc(-c4ccc5c(c4)oc4ccc(-c6ccccc6-c6ccc7oc8ccccc8c7c6)cc45)c3)n2)cc1 ; c1ccc(-c2nc(-c3ccccc3)nc(-c3cccc(-c4ccc5c(c4)oc4ccc(-c6ccccc6-c6ccc7c(c6)oc6ccccc67)cc45)c3)n2)cc1]

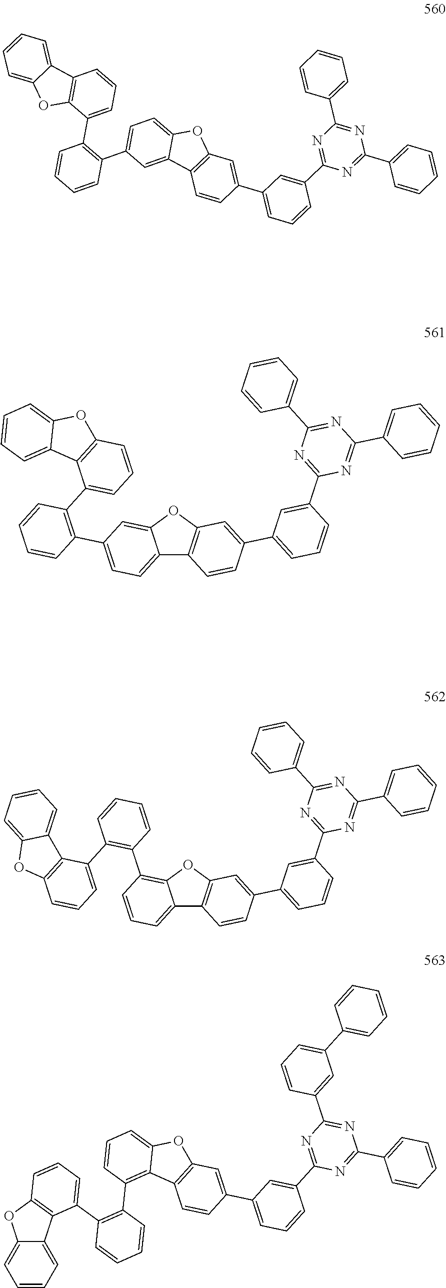 [SMILES: c1ccc(-c2nc(-c3ccccc3)nc(-c3cccc(-c4ccc5c(c4)oc4ccc(-c6ccccc6-c6cccc7c6oc6ccccc67)cc45)c3)n2)cc1 ; c1ccc(-c2nc(-c3ccccc3)nc(-c3cccc(-c4ccc5c(c4)oc4cc(-c6ccccc6-c6cccc7oc8ccccc8c67)ccc45)c3)n2)cc1 ; c1ccc(-c2nc(-c3ccccc3)nc(-c3cccc(-c4ccc5c(c4)oc4c(-c6ccccc6-c6cccc7oc8ccccc8c67)cccc45)c3)n2)cc1 ; c1ccc(-c2cccc(-c3nc(-c4ccccc4)nc(-c4cccc(-c5ccc6c(c5)oc5cccc(-c7ccccc7-c7cccc8oc9ccccc9c78)c56)c4)n3)c2)cc1]

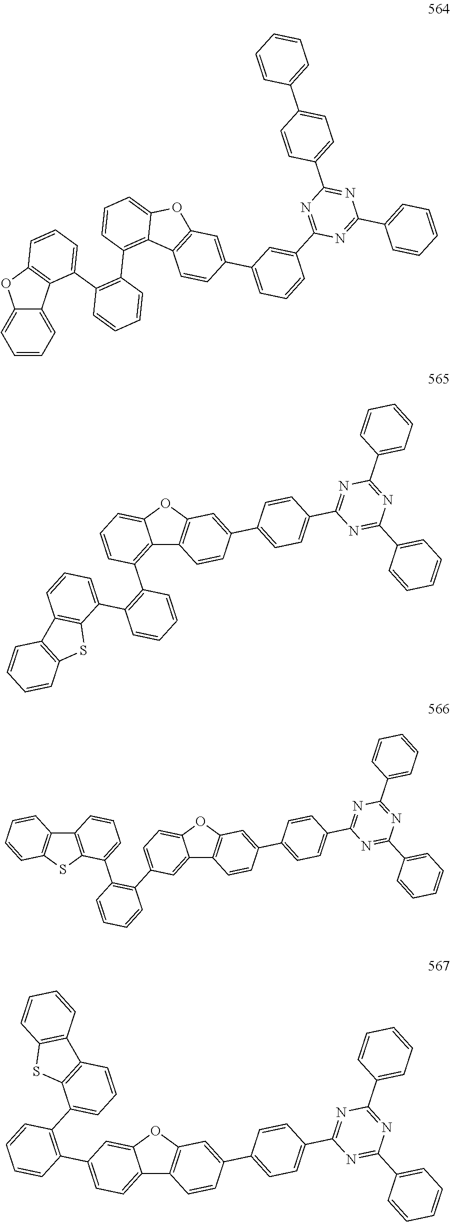 [SMILES: c1ccc(-c2ccc(-c3nc(-c4ccccc4)nc(-c4cccc(-c5ccc6c(c5)oc5cccc(-c7ccccc7-c7cccc8oc9ccccc9c78)c56)c4)n3)cc2)cc1 ; c1ccc(-c2nc(-c3ccccc3)nc(-c3ccc(-c4ccc5c(c4)oc4cccc(-c6ccccc6-c6cccc7c6sc6ccccc67)c45)cc3)n2)cc1 ; c1ccc(-c2nc(-c3ccccc3)nc(-c3ccc(-c4ccc5c(c4)oc4ccc(-c6ccccc6-c6cccc7c6sc6ccccc67)cc45)cc3)n2)cc1 ; c1ccc(-c2nc(-c3ccccc3)nc(-c3ccc(-c4ccc5c(c4)oc4cc(-c6ccccc6-c6cccc7c6sc6ccccc67)ccc45)cc3)n2)cc1]

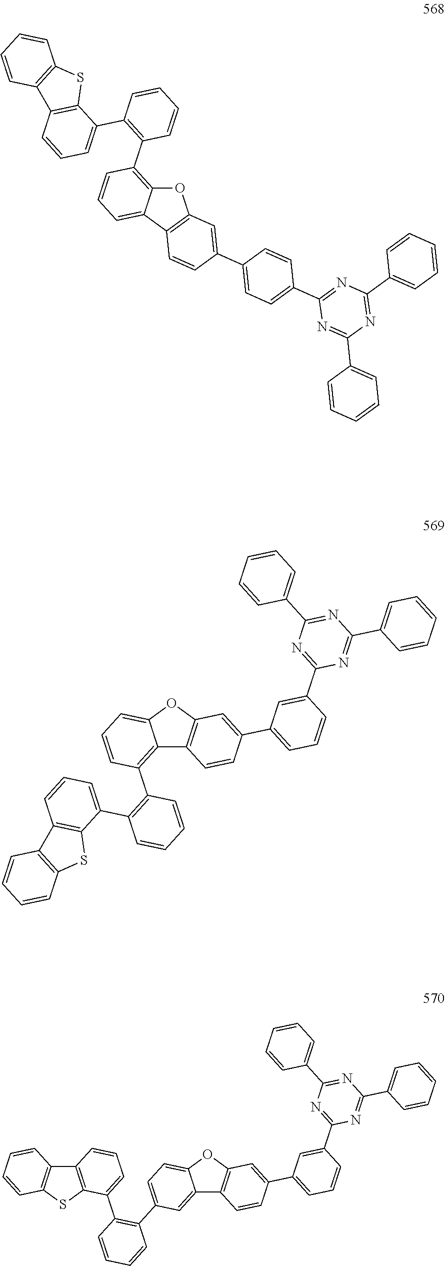 [SMILES: c1ccc(-c2nc(-c3ccccc3)nc(-c3ccc(-c4ccc5c(c4)oc4c(-c6ccccc6-c6cccc7c6sc6ccccc67)cccc45)cc3)n2)cc1 ; c1ccc(-c2nc(-c3ccccc3)nc(-c3cccc(-c4ccc5c(c4)oc4cccc(-c6ccccc6-c6cccc7c6sc6ccccc67)c45)c3)n2)cc1 ; c1ccc(-c2nc(-c3ccccc3)nc(-c3cccc(-c4ccc5c(c4)oc4ccc(-c6ccccc6-c6cccc7c6sc6ccccc67)cc45)c3)n2)cc1]

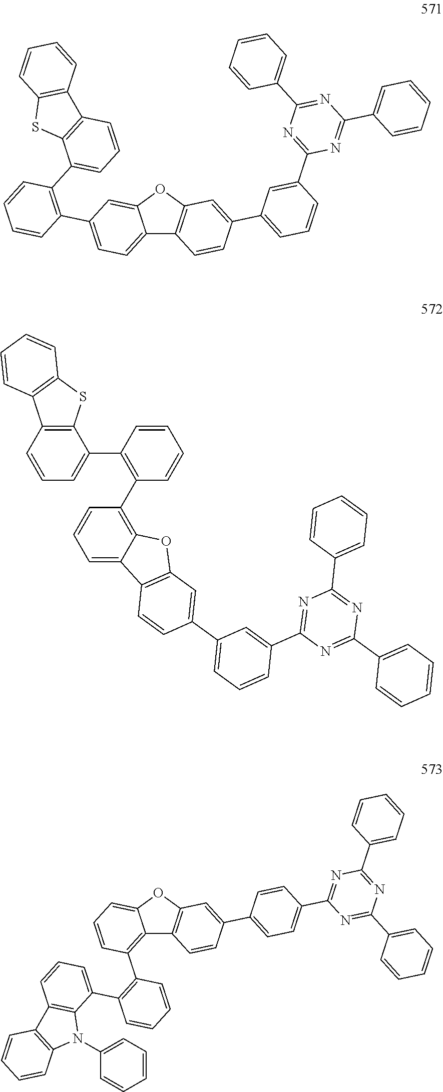 [SMILES: c1ccc(-c2nc(-c3ccccc3)nc(-c3cccc(-c4ccc5c(c4)oc4cc(-c6ccccc6-c6cccc7c6sc6ccccc67)ccc45)c3)n2)cc1 ; c1ccc(-c2nc(-c3ccccc3)nc(-c3cccc(-c4ccc5c(c4)oc4c(-c6ccccc6-c6cccc7c6sc6ccccc67)cccc45)c3)n2)cc1 ; c1ccc(-c2nc(-c3ccccc3)nc(-c3ccc(-c4ccc5c(c4)oc4cccc(-c6ccccc6-c6cccc7c8ccccc8n(-c8ccccc8)c67)c45)cc3)n2)cc1]

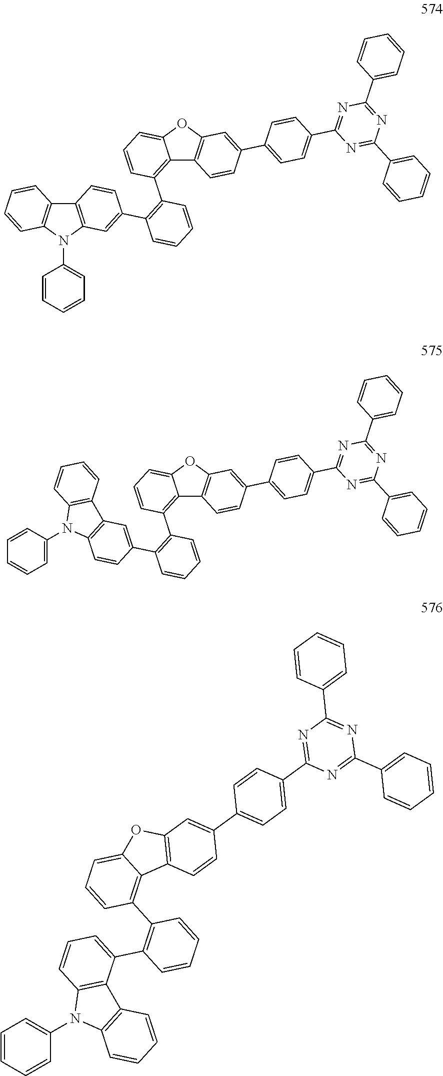 [SMILES: c1ccc(-c2nc(-c3ccccc3)nc(-c3ccc(-c4ccc5c(c4)oc4cccc(-c6ccccc6-c6ccc7c8ccccc8n(-c8ccccc8)c7c6)c45)cc3)n2)cc1 ; c1ccc(-c2nc(-c3ccccc3)nc(-c3ccc(-c4ccc5c(c4)oc4cccc(-c6ccccc6-c6ccc7c(c6)c6ccccc6n7-c6ccccc6)c45)cc3)n2)cc1 ; c1ccc(-c2nc(-c3ccccc3)nc(-c3ccc(-c4ccc5c(c4)oc4cccc(-c6ccccc6-c6cccc7c6c6ccccc6n7-c6ccccc6)c45)cc3)n2)cc1]

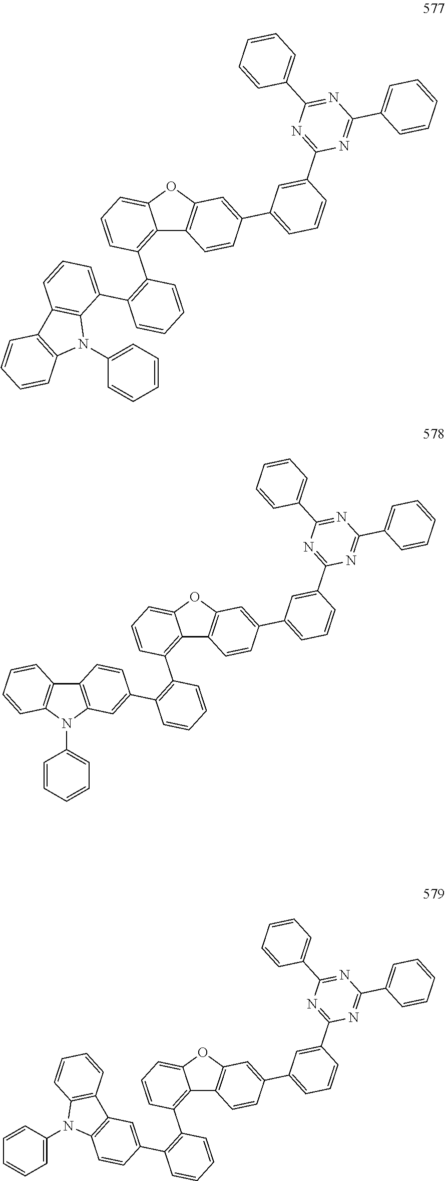 [SMILES: c1ccc(-c2nc(-c3ccccc3)nc(-c3cccc(-c4ccc5c(c4)oc4cccc(-c6ccccc6-c6cccc7c8ccccc8n(-c8ccccc8)c67)c45)c3)n2)cc1 ; c1ccc(-c2nc(-c3ccccc3)nc(-c3cccc(-c4ccc5c(c4)oc4cccc(-c6ccccc6-c6ccc7c8ccccc8n(-c8ccccc8)c7c6)c45)c3)n2)cc1 ; c1ccc(-c2nc(-c3ccccc3)nc(-c3cccc(-c4ccc5c(c4)oc4cccc(-c6ccccc6-c6ccc7c(c6)c6ccccc6n7-c6ccccc6)c45)c3)n2)cc1]

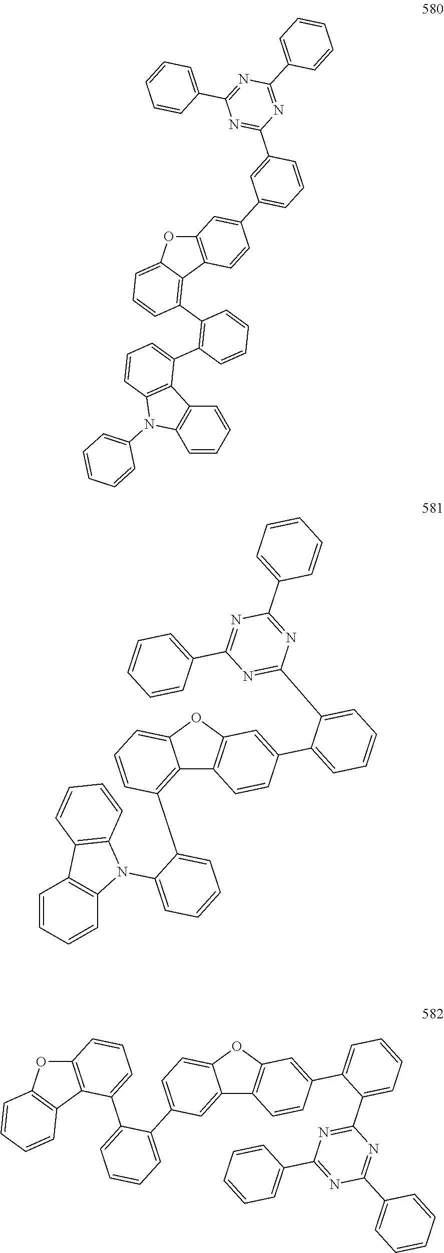 [SMILES: c1ccc(-c2nc(-c3ccccc3)nc(-c3cccc(-c4ccc5c(c4)oc4cccc(-c6ccccc6-c6cccc7c6c6ccccc6n7-c6ccccc6)c45)c3)n2)cc1 ; c1ccc(-c2nc(-c3ccccc3)nc(-c3ccccc3-c3ccc4c(c3)oc3cccc(-c5ccccc5-n5c6ccccc6c6ccccc65)c34)n2)cc1 ; c1ccc(-c2nc(-c3ccccc3)nc(-c3ccccc3-c3ccc4c(c3)oc3ccc(-c5ccccc5-c5cccc6oc7ccccc7c56)cc34)n2)cc1]

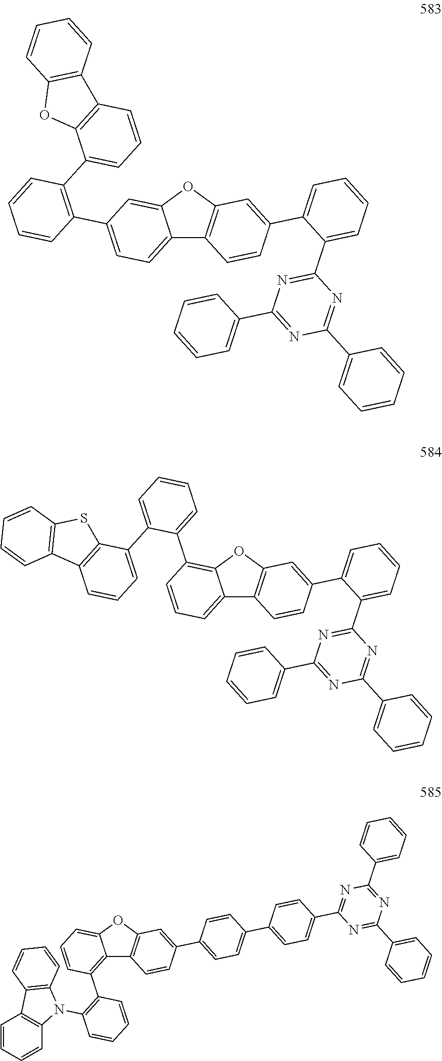 [SMILES: c1ccc(-c2nc(-c3ccccc3)nc(-c3ccccc3-c3ccc4c(c3)oc3cc(-c5ccccc5-c5cccc6c5oc5ccccc56)ccc34)n2)cc1 ; c1ccc(-c2nc(-c3ccccc3)nc(-c3ccccc3-c3ccc4c(c3)oc3c(-c5ccccc5-c5cccc6c5sc5ccccc56)cccc34)n2)cc1 ; c1ccc(-c2nc(-c3ccccc3)nc(-c3ccc(-c4ccc(-c5ccc6c(c5)oc5cccc(-c7ccccc7-n7c8ccccc8c8ccccc87)c56)cc4)cc3)n2)cc1]

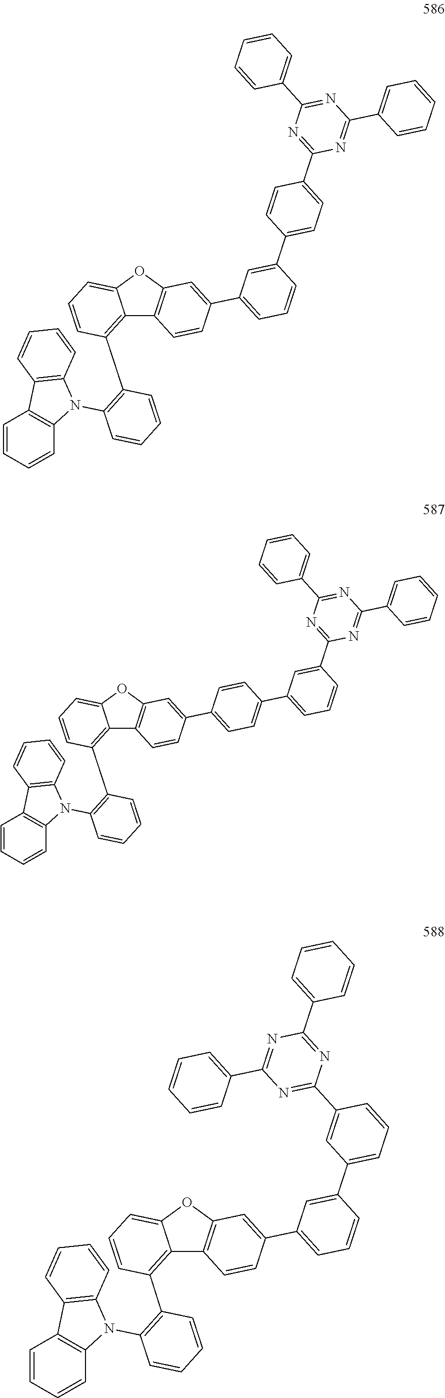 [SMILES: c1ccc(-c2nc(-c3ccccc3)nc(-c3ccc(-c4cccc(-c5ccc6c(c5)oc5cccc(-c7ccccc7-n7c8ccccc8c8ccccc87)c56)c4)cc3)n2)cc1 ; c1ccc(-c2nc(-c3ccccc3)nc(-c3cccc(-c4ccc(-c5ccc6c(c5)oc5cccc(-c7ccccc7-n7c8ccccc8c8ccccc87)c56)cc4)c3)n2)cc1 ; c1ccc(-c2nc(-c3ccccc3)nc(-c3cccc(-c4cccc(-c5ccc6c(c5)oc5cccc(-c7ccccc7-n7c8ccccc8c8ccccc87)c56)c4)c3)n2)cc1]

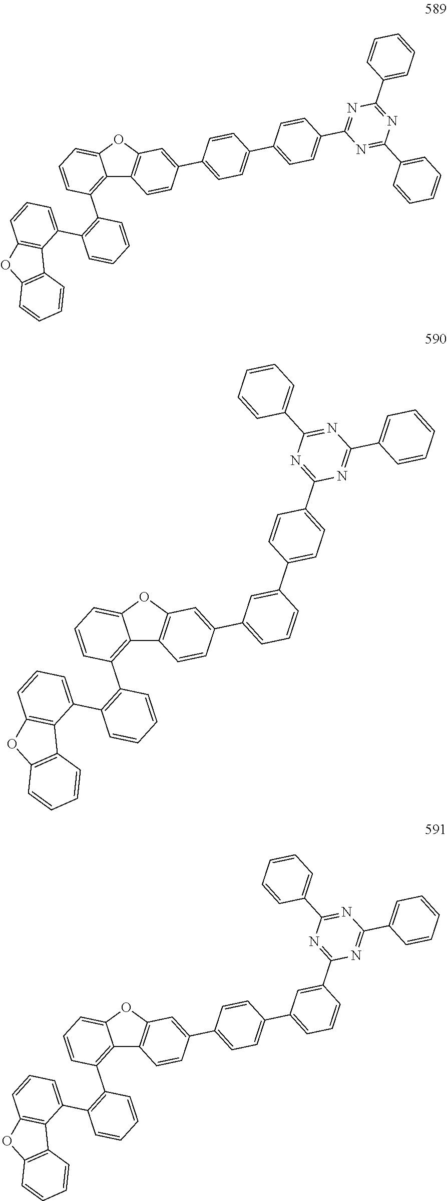 [SMILES: c1ccc(-c2nc(-c3ccccc3)nc(-c3ccc(-c4ccc(-c5ccc6c(c5)oc5cccc(-c7ccccc7-c7cccc8oc9ccccc9c78)c56)cc4)cc3)n2)cc1 ; c1ccc(-c2nc(-c3ccccc3)nc(-c3ccc(-c4cccc(-c5ccc6c(c5)oc5cccc(-c7ccccc7-c7cccc8oc9ccccc9c78)c56)c4)cc3)n2)cc1 ; c1ccc(-c2nc(-c3ccccc3)nc(-c3cccc(-c4ccc(-c5ccc6c(c5)oc5cccc(-c7ccccc7-c7cccc8oc9ccccc9c78)c56)cc4)c3)n2)cc1]

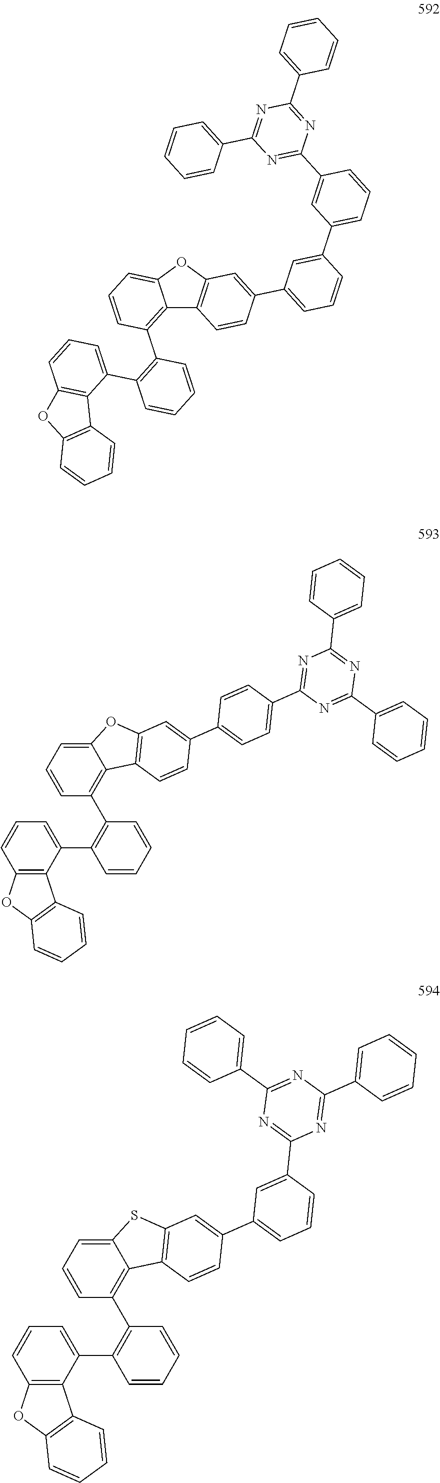 [SMILES: c1ccc(-c2nc(-c3ccccc3)nc(-c3cccc(-c4cccc(-c5ccc6c(c5)oc5cccc(-c7ccccc7-c7cccc8oc9ccccc9c78)c56)c4)c3)n2)cc1 ; c1ccc(-c2nc(-c3ccccc3)nc(-c3ccc(-c4ccc5c(c4)oc4cccc(-c6ccccc6-c6cccc7oc8ccccc8c67)c45)cc3)n2)cc1 ; c1ccc(-c2nc(-c3ccccc3)nc(-c3cccc(-c4ccc5c(c4)sc4cccc(-c6ccccc6-c6cccc7oc8ccccc8c67)c45)c3)n2)cc1]

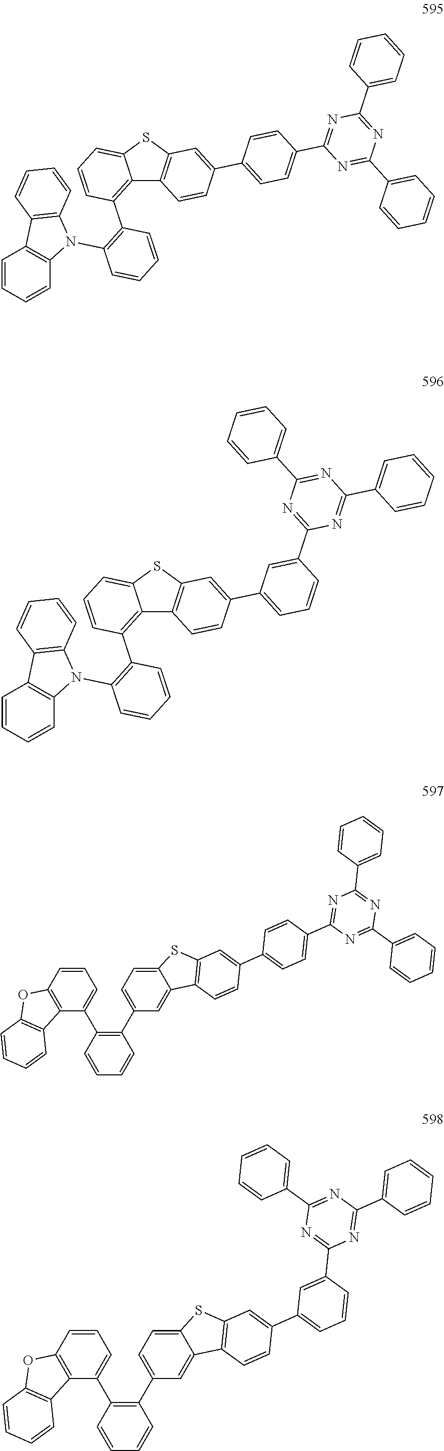 [SMILES: c1ccc(-c2nc(-c3ccccc3)nc(-c3ccc(-c4ccc5c(c4)sc4cccc(-c6ccccc6-n6c7ccccc7c7ccccc76)c45)cc3)n2)cc1 ; c1ccc(-c2nc(-c3ccccc3)nc(-c3cccc(-c4ccc5c(c4)sc4cccc(-c6ccccc6-n6c7ccccc7c7ccccc76)c45)c3)n2)cc1 ; c1ccc(-c2nc(-c3ccccc3)nc(-c3ccc(-c4ccc5c(c4)sc4ccc(-c6ccccc6-c6cccc7oc8ccccc8c67)cc45)cc3)n2)cc1 ; c1ccc(-c2nc(-c3ccccc3)nc(-c3cccc(-c4ccc5c(c4)sc4ccc(-c6ccccc6-c6cccc7oc8ccccc8c67)cc45)c3)n2)cc1]

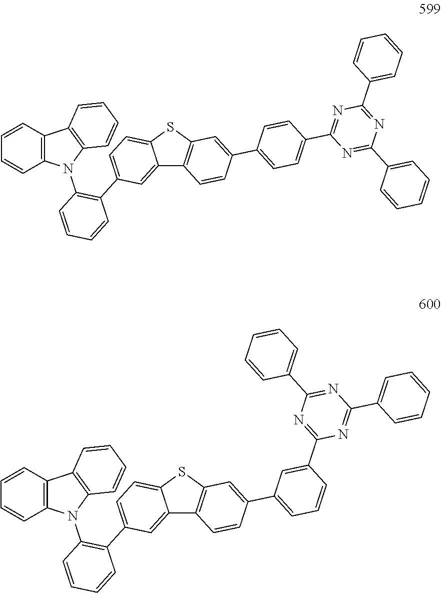 [SMILES: c1ccc(-c2nc(-c3ccccc3)nc(-c3ccc(-c4ccc5c(c4)sc4ccc(-c6ccccc6-n6c7ccccc7c7ccccc76)cc45)cc3)n2)cc1 ; c1ccc(-c2nc(-c3ccccc3)nc(-c3cccc(-c4ccc5c(c4)sc4ccc(-c6ccccc6-n6c7ccccc7c7ccccc76)cc45)c3)n2)cc1]

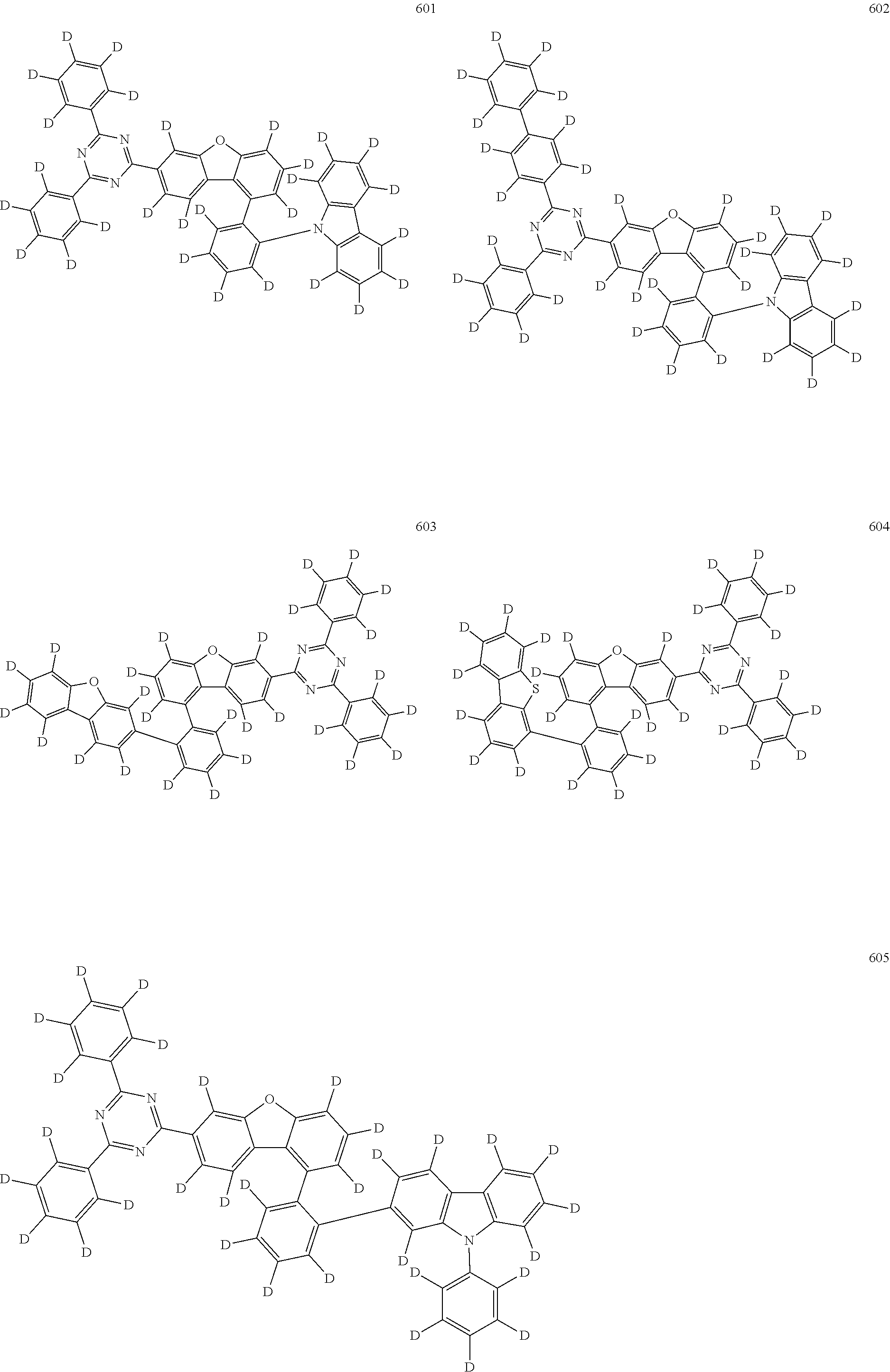

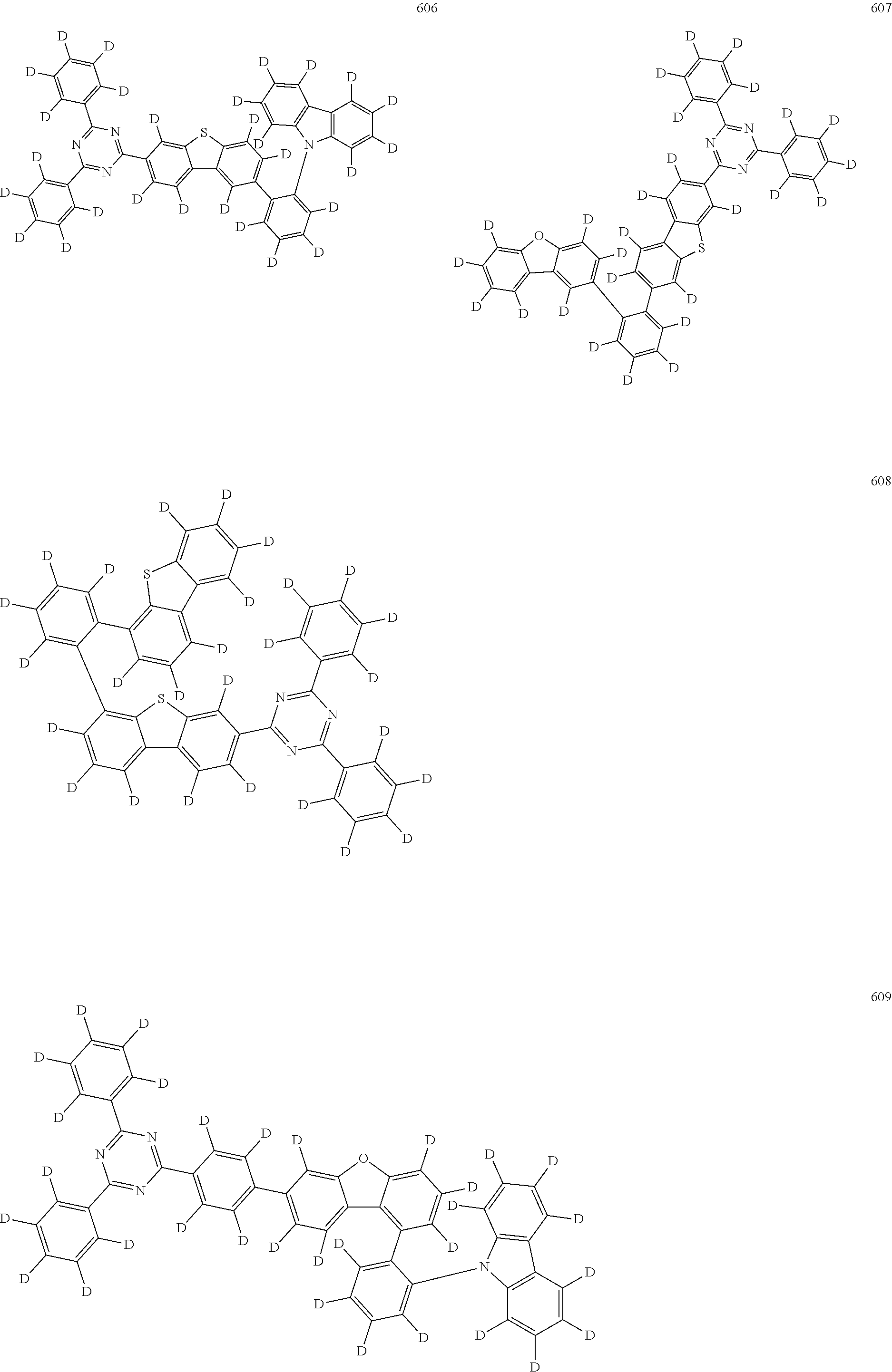

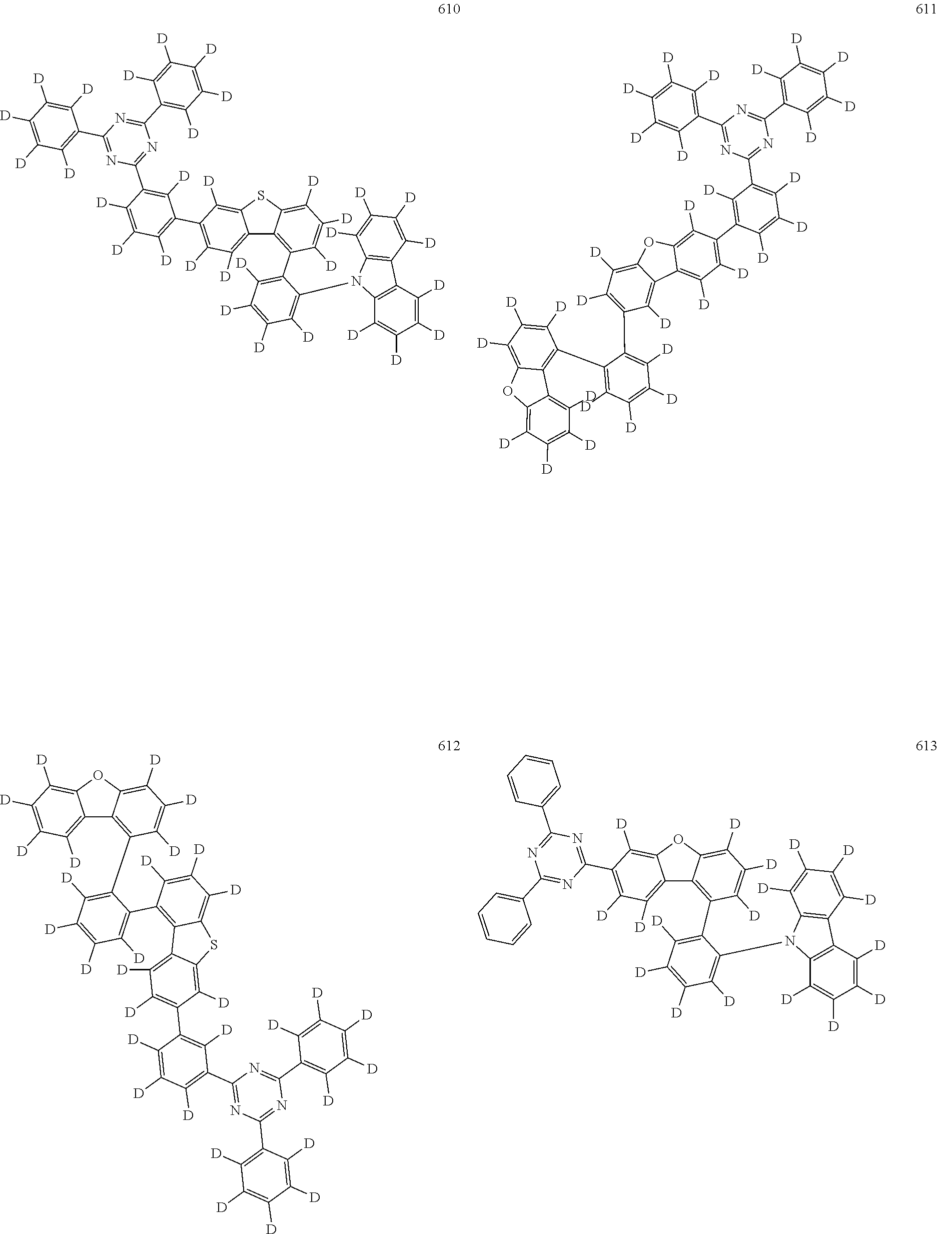

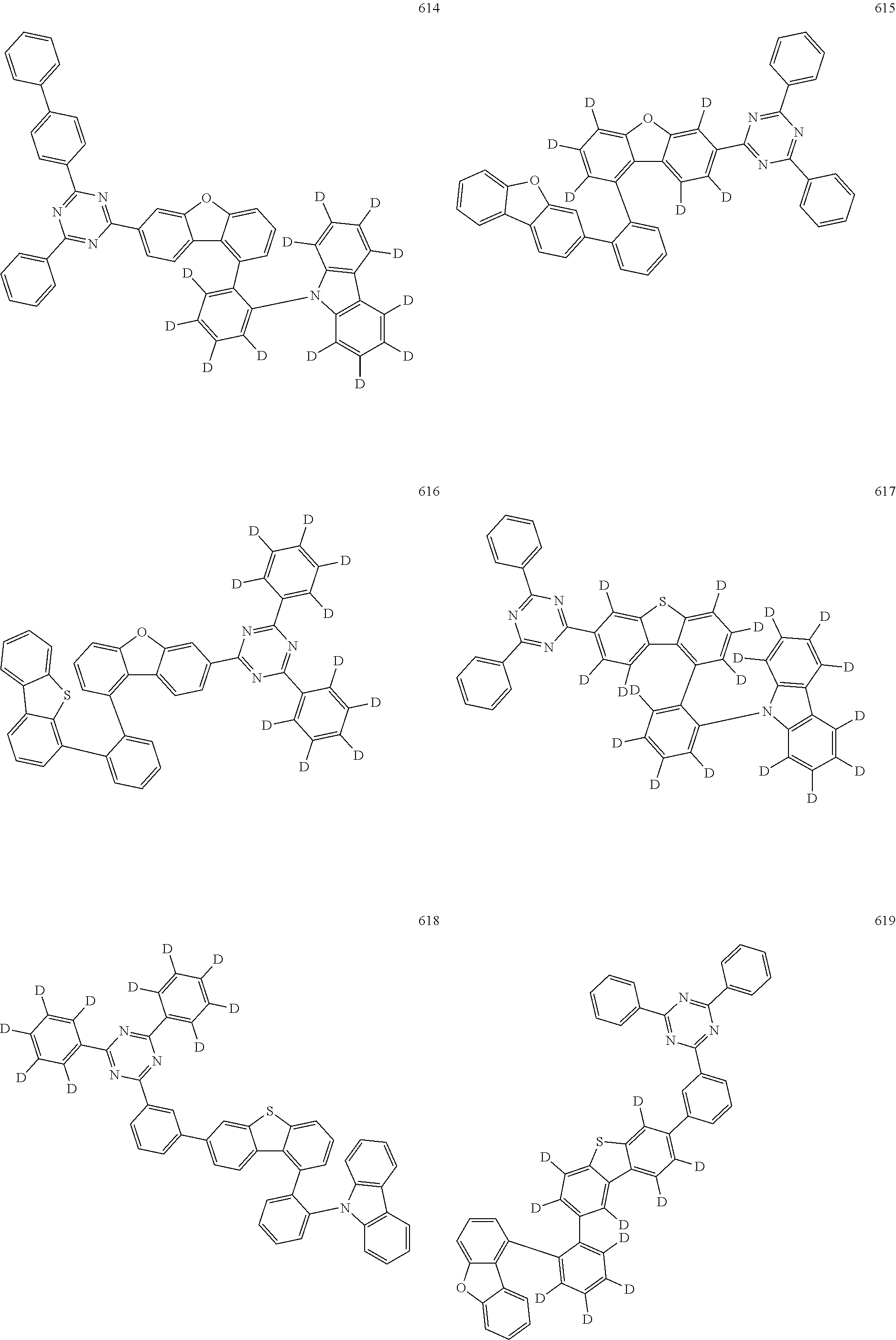

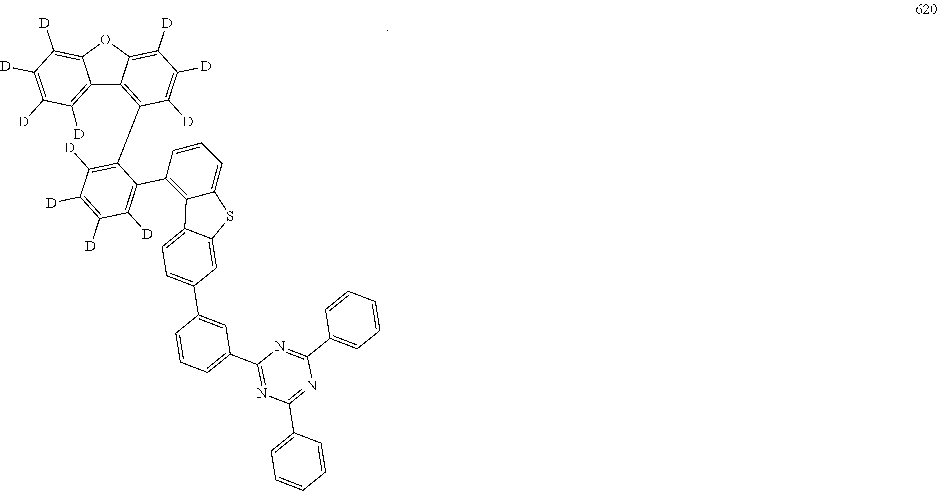 [SMILES: [2H]c1c([2H])c([2H])c(-c2c([2H])c([2H])c([2H])c3oc4c([2H])c([2H])c([2H])c([2H])c4c23)c(-c2cccc3sc4cc(-c5cccc(-c6nc(-c7ccccc7)nc(-c7ccccc7)n6)c5)ccc4c23)c1[2H]]

7. An organic light emitting device comprising:
a first electrode;
a second electrode; and
an organic material layer having one or more layers provided between the first electrode and the second electrode,
wherein one or more layers of the organic material layer comprise one or more of the heterocyclic compounds of claim 1.

8. The organic light emitting device of claim 7, wherein the organic material layer comprises a light emitting layer, and the light emitting layer comprises one or more of the heterocyclic compounds.

9. The organic light emitting device of claim 7, wherein the organic material layer comprises a light emitting layer, the light emitting layer comprises a host, and the host comprises one or more of the heterocyclic compounds.

10. The organic light emitting device of claim 7, wherein the organic material layer comprising the heterocyclic compound further comprises a compound of the following Chemical Formula 2: 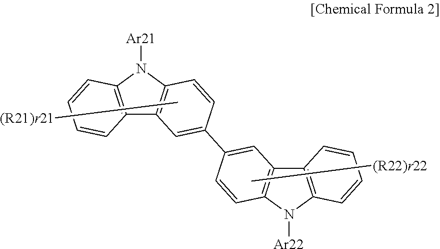
in Chemical Formula 2,
Ar21 and Ar22 are each independently a substituted or unsubstituted C6 to C60 aryl group; or a substituted or unsubstituted C2 to C60 heteroaryl group,
R21 and R22 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
r21 and r22 are each an integer from 0 to 7, and when r21 and r22 are each 2 or higher, substituents in the parenthesis are the same as or different from each other.

11. The organic light emitting device of claim 10, wherein the deuterium content of Chemical Formula 2 is 0% or 20% to 100%.

12. The organic light emitting device of claim 10, wherein Chemical Formula 2 is represented by any one of the following compounds:

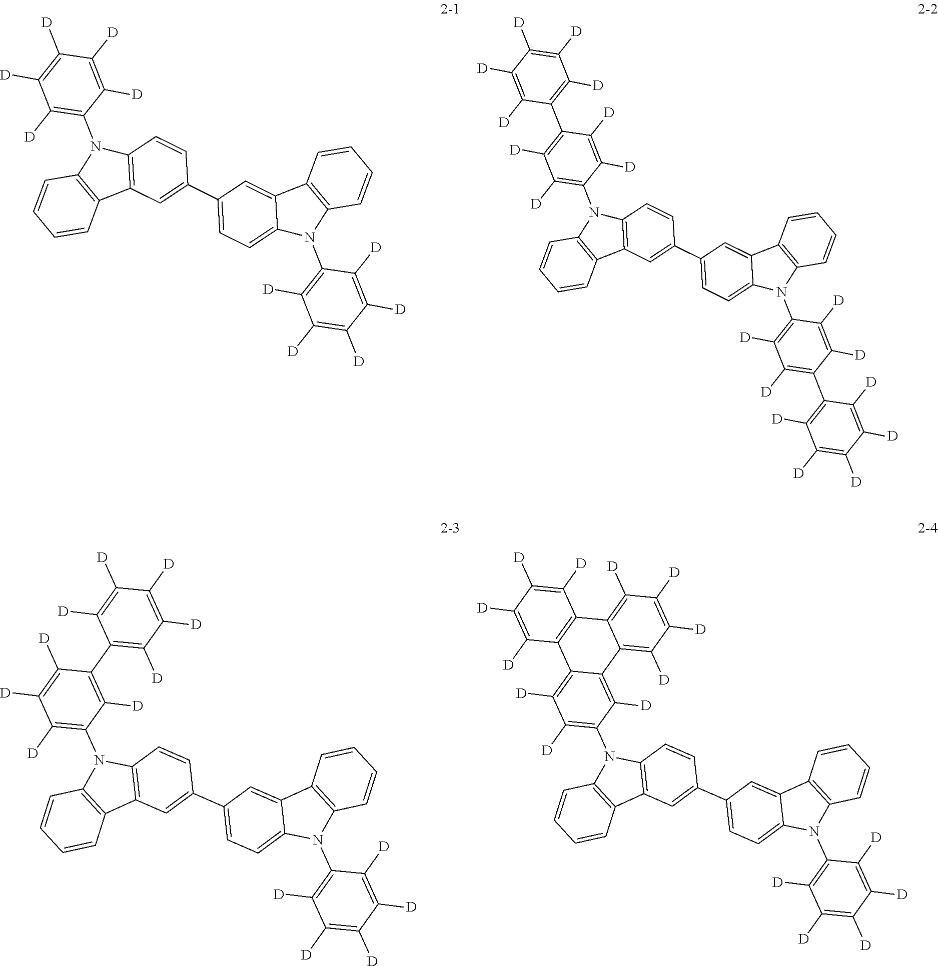

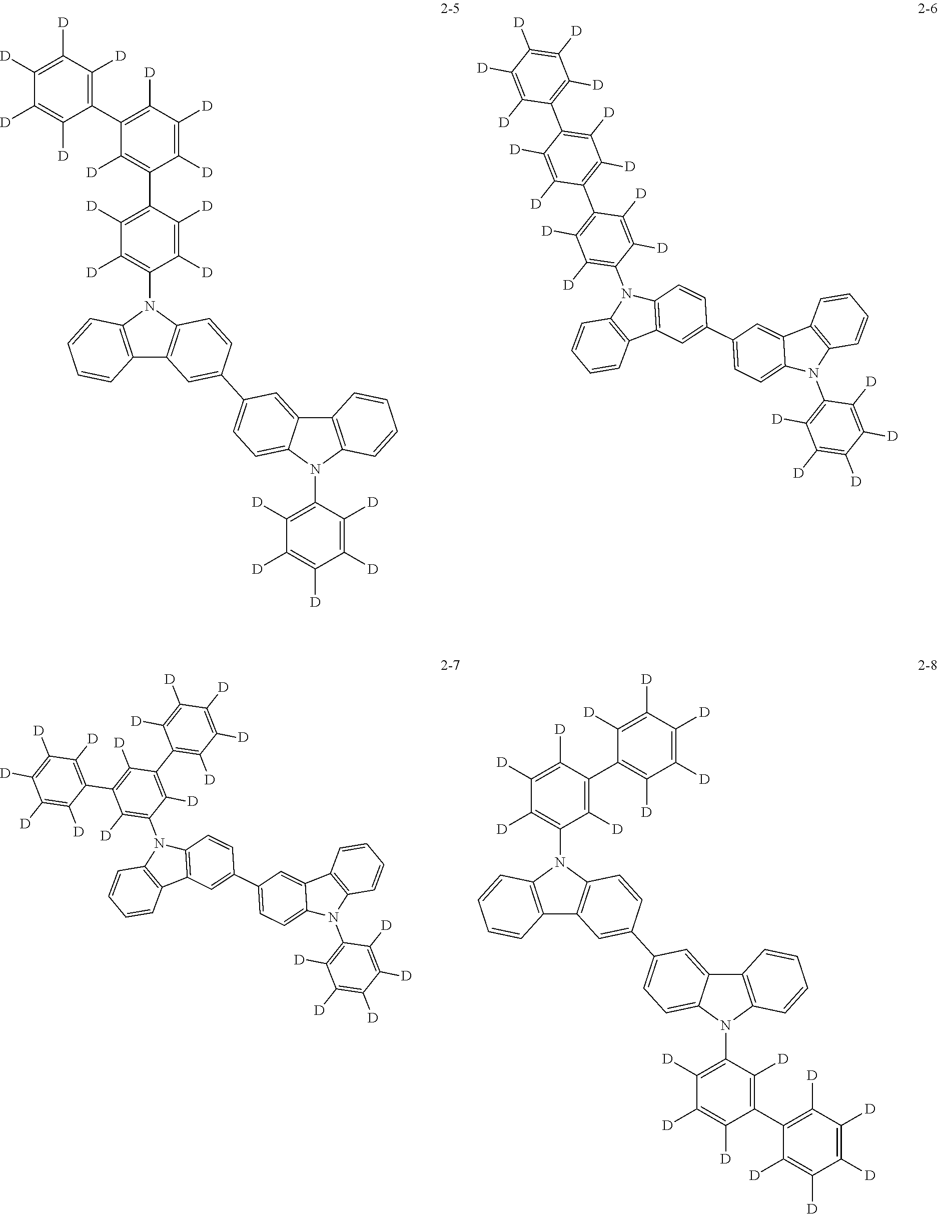

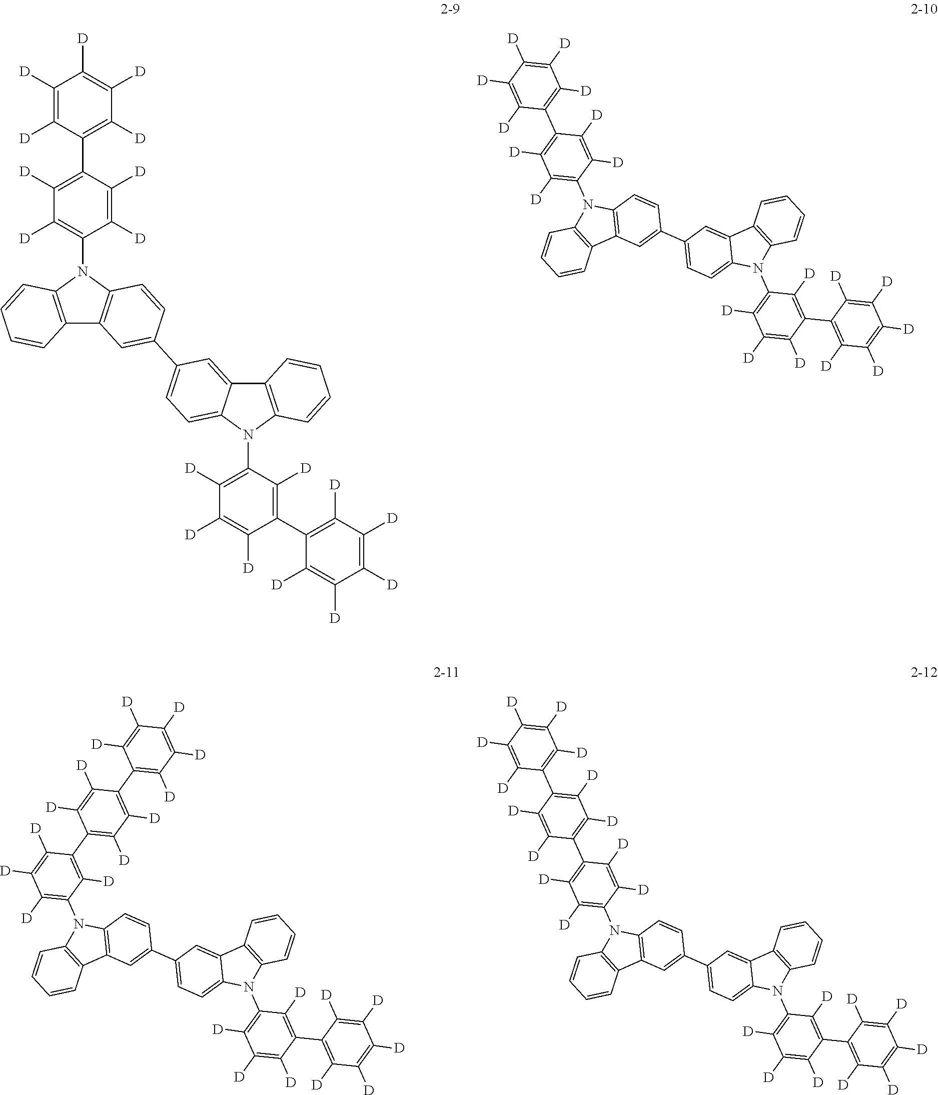

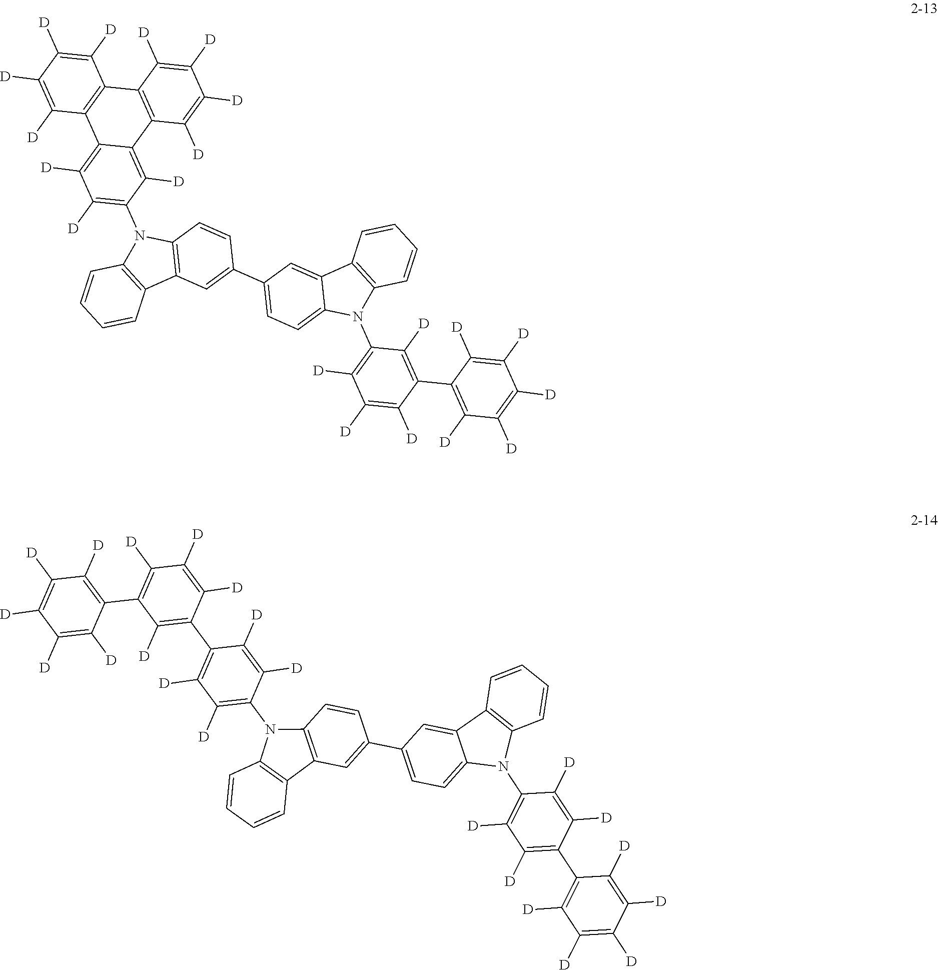 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c6c7c([2H])c([2H])c([2H])c([2H])c7c7c([2H])c([2H])c([2H])c([2H])c7c6c5[2H])ccc43)c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c(-c7c([2H])c([2H])c([2H])c([2H])c7[2H])c6[2H])c([2H])c5[2H])ccc43)c([2H])c2[2H])c([2H])c1[2H]]

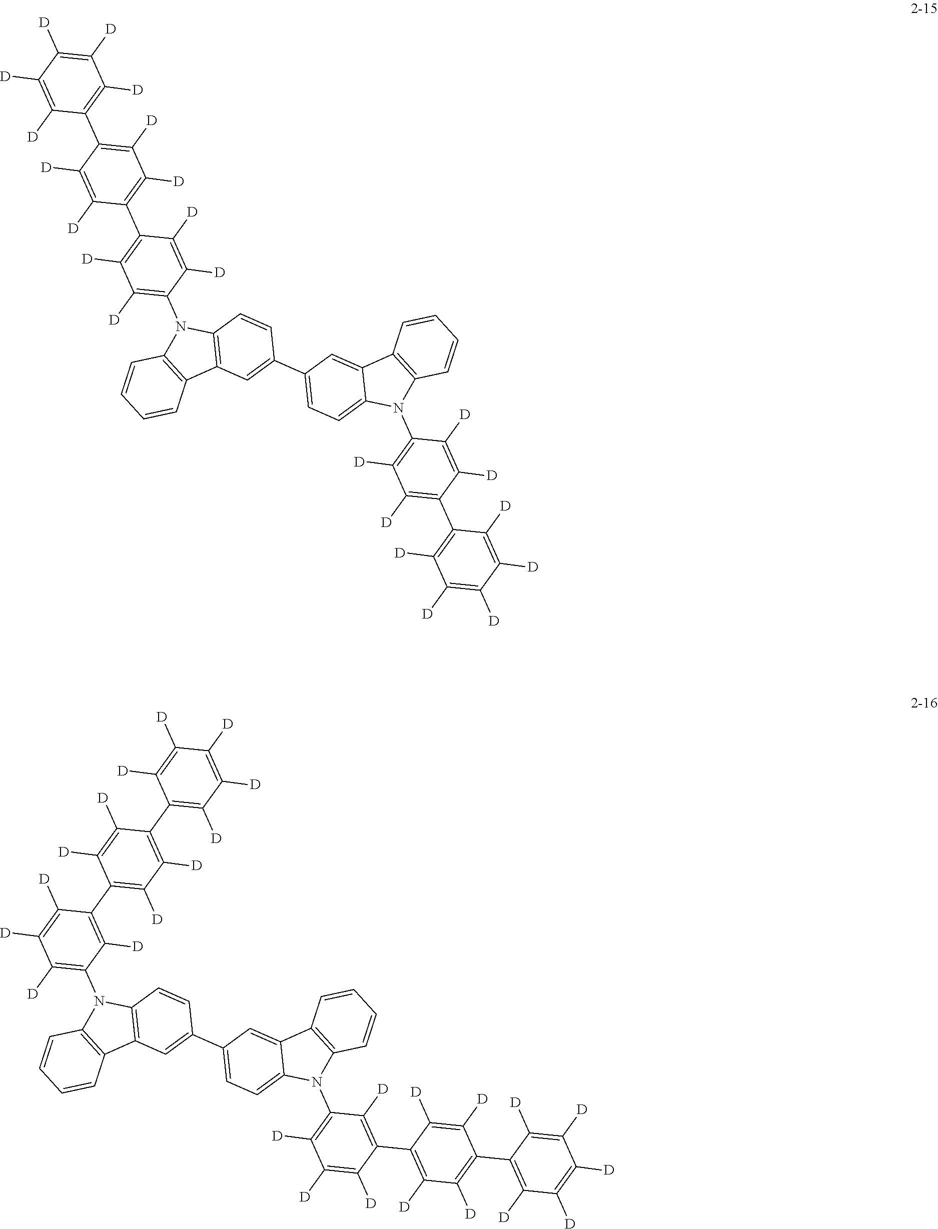 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c([2H])c([2H])c7[2H])c([2H])c6[2H])ccc54)c([2H])c3[2H])c([2H])c2[2H])c([2H])c1[2H] ; [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c(-c8c([2H])c([2H])c([2H])c([2H])c8[2H])c([2H])c7[2H])c6[2H])ccc54)c3[2H])c([2H])c2[2H])c([2H])c1[2H]]

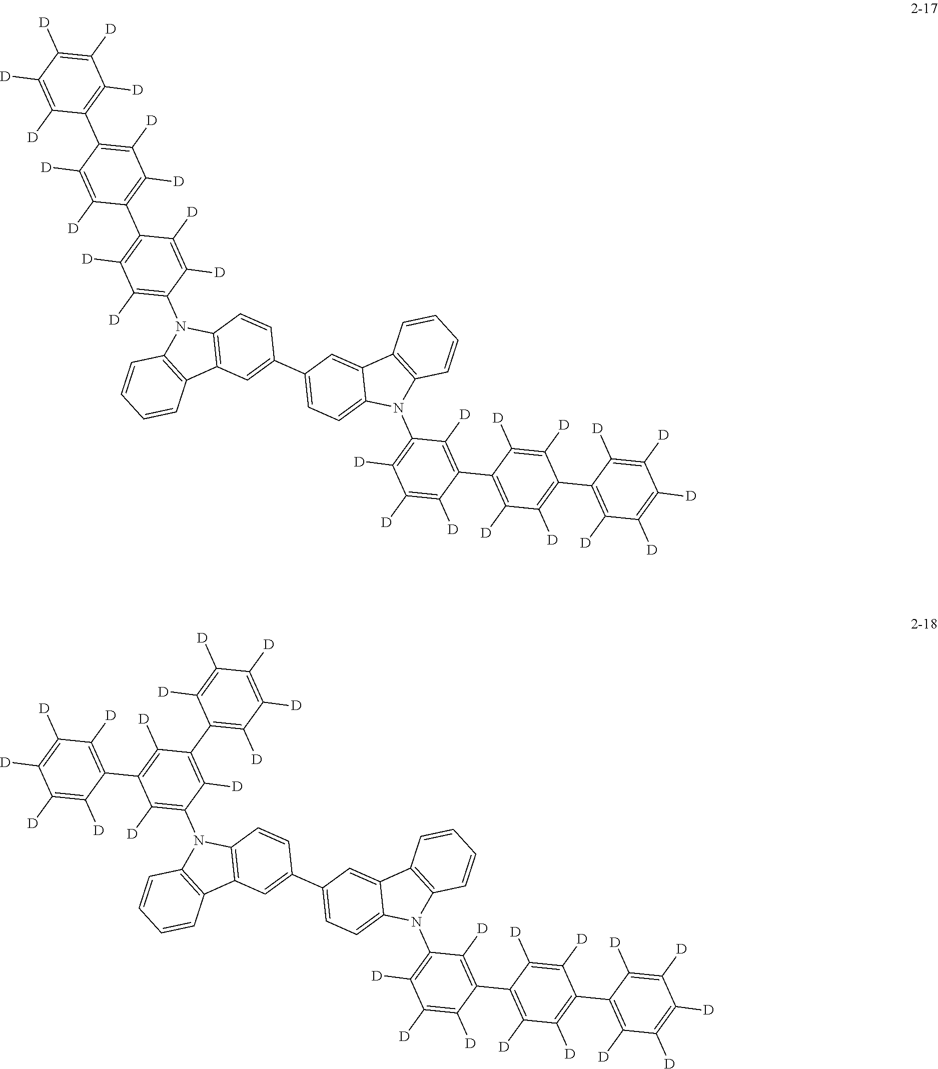

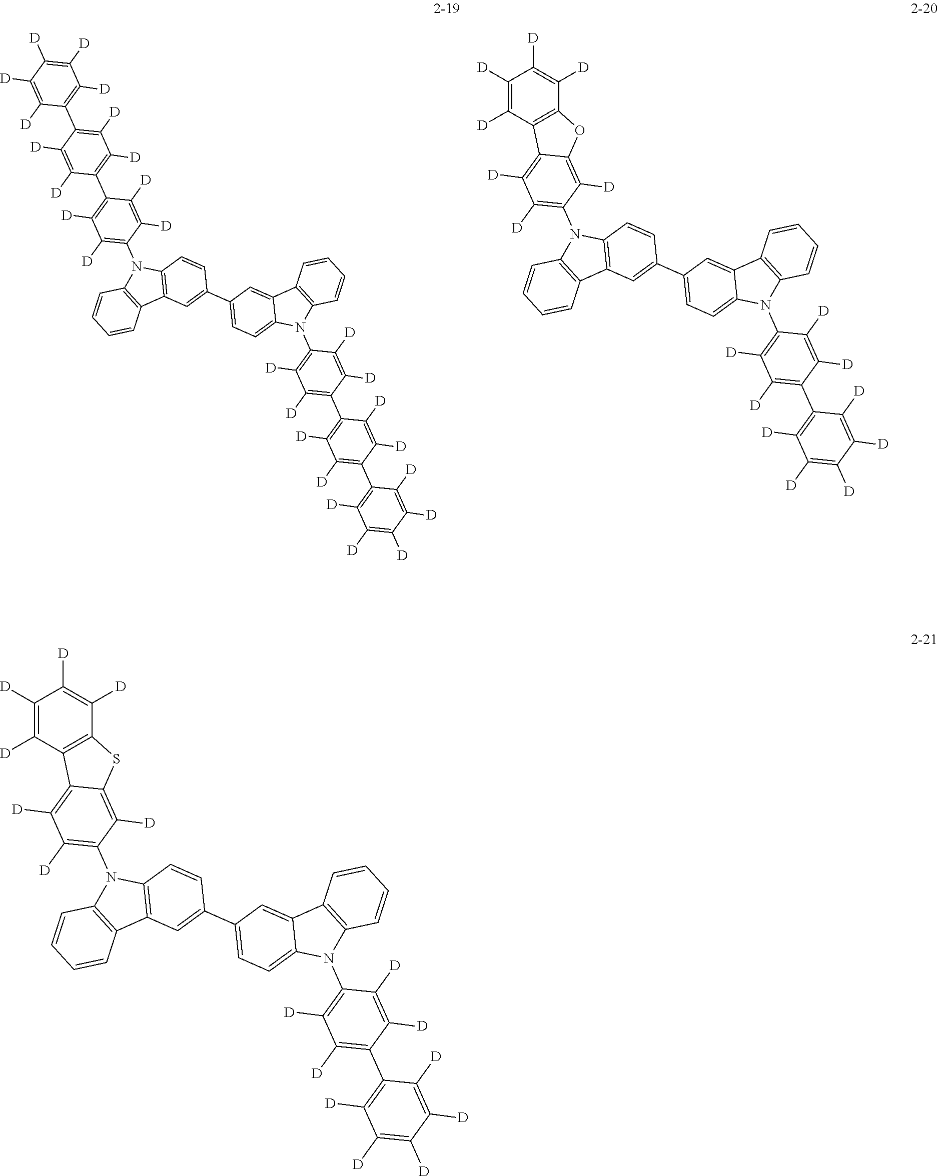

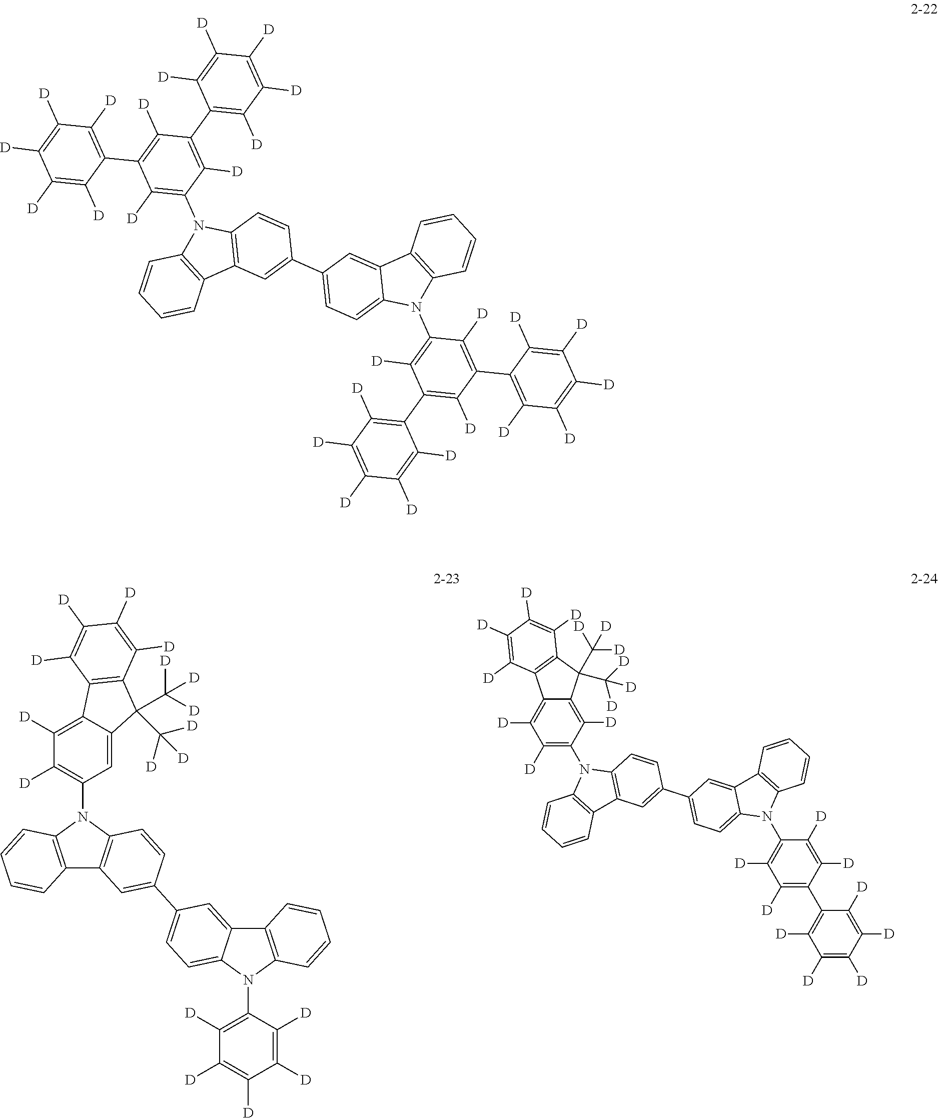

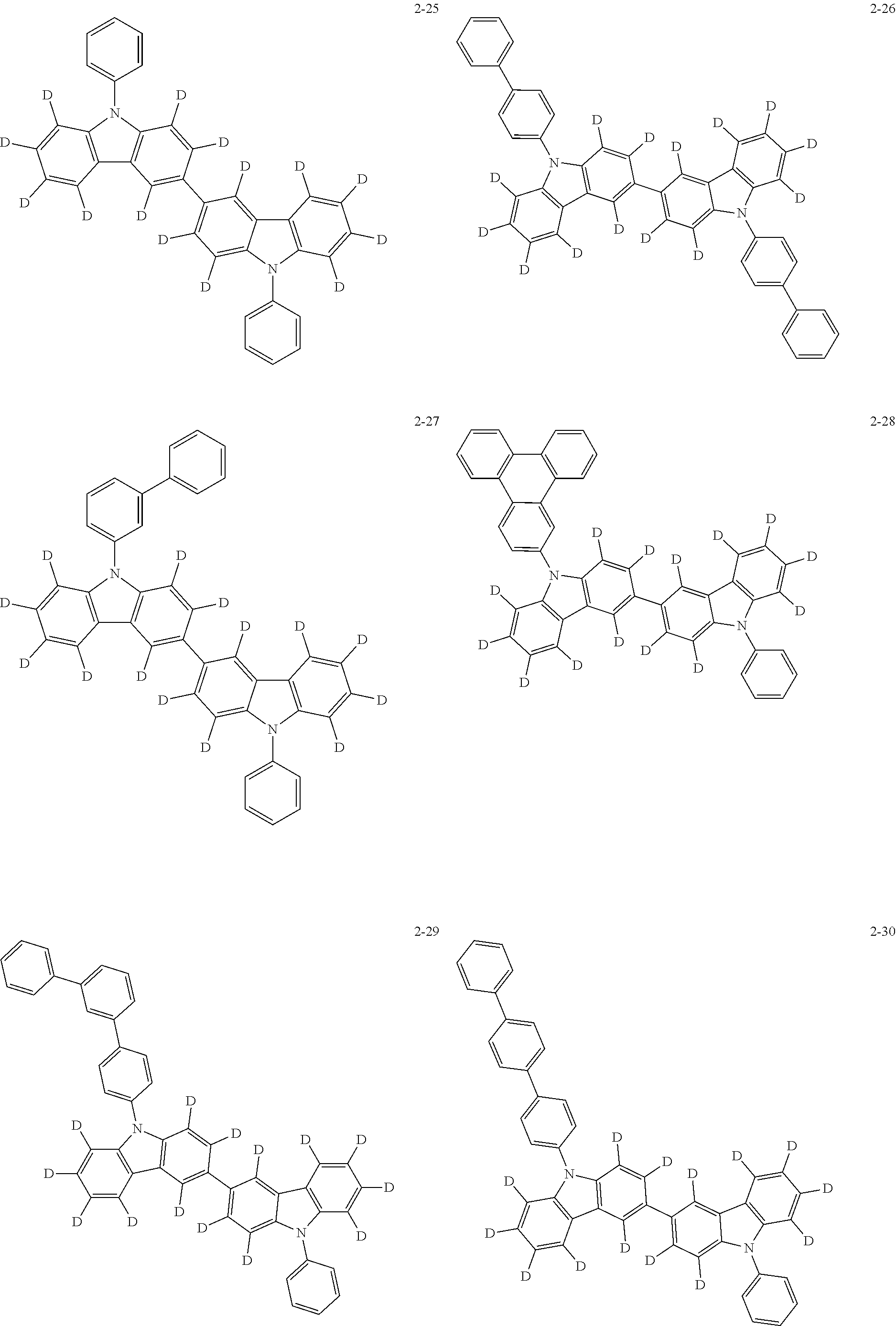

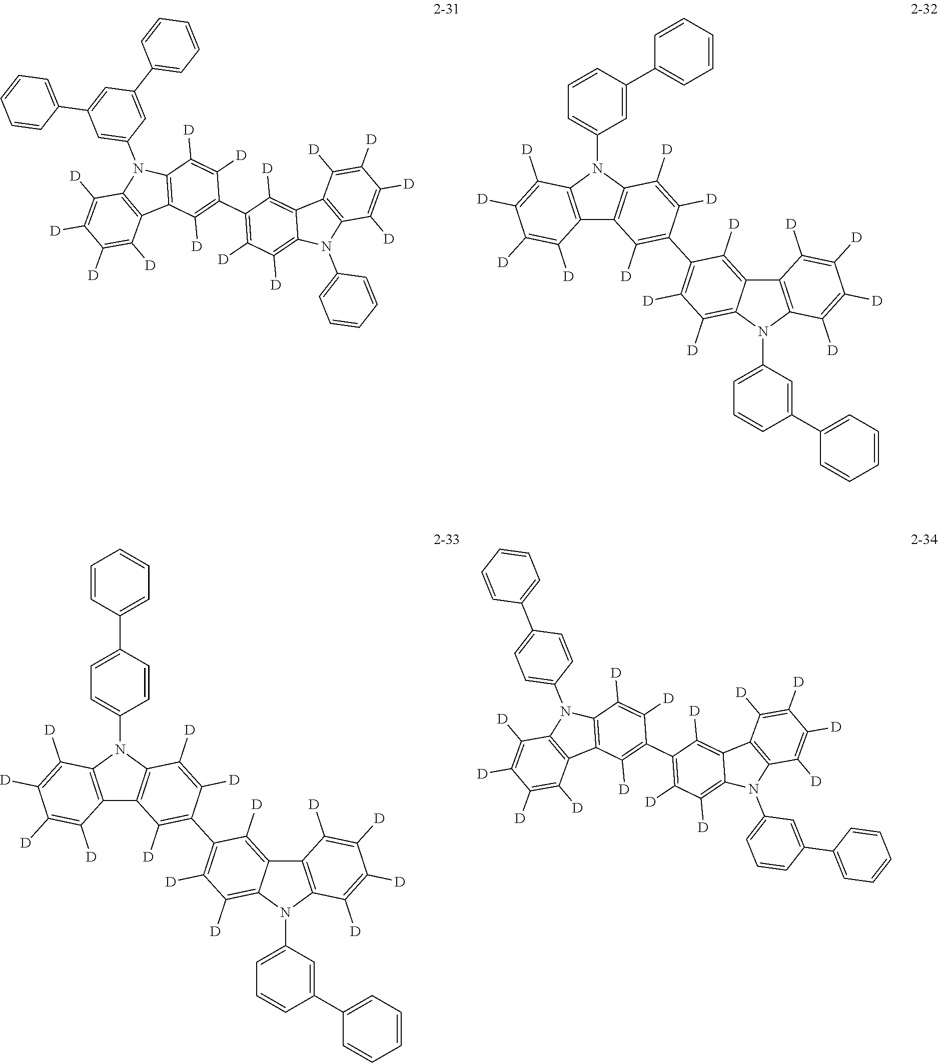

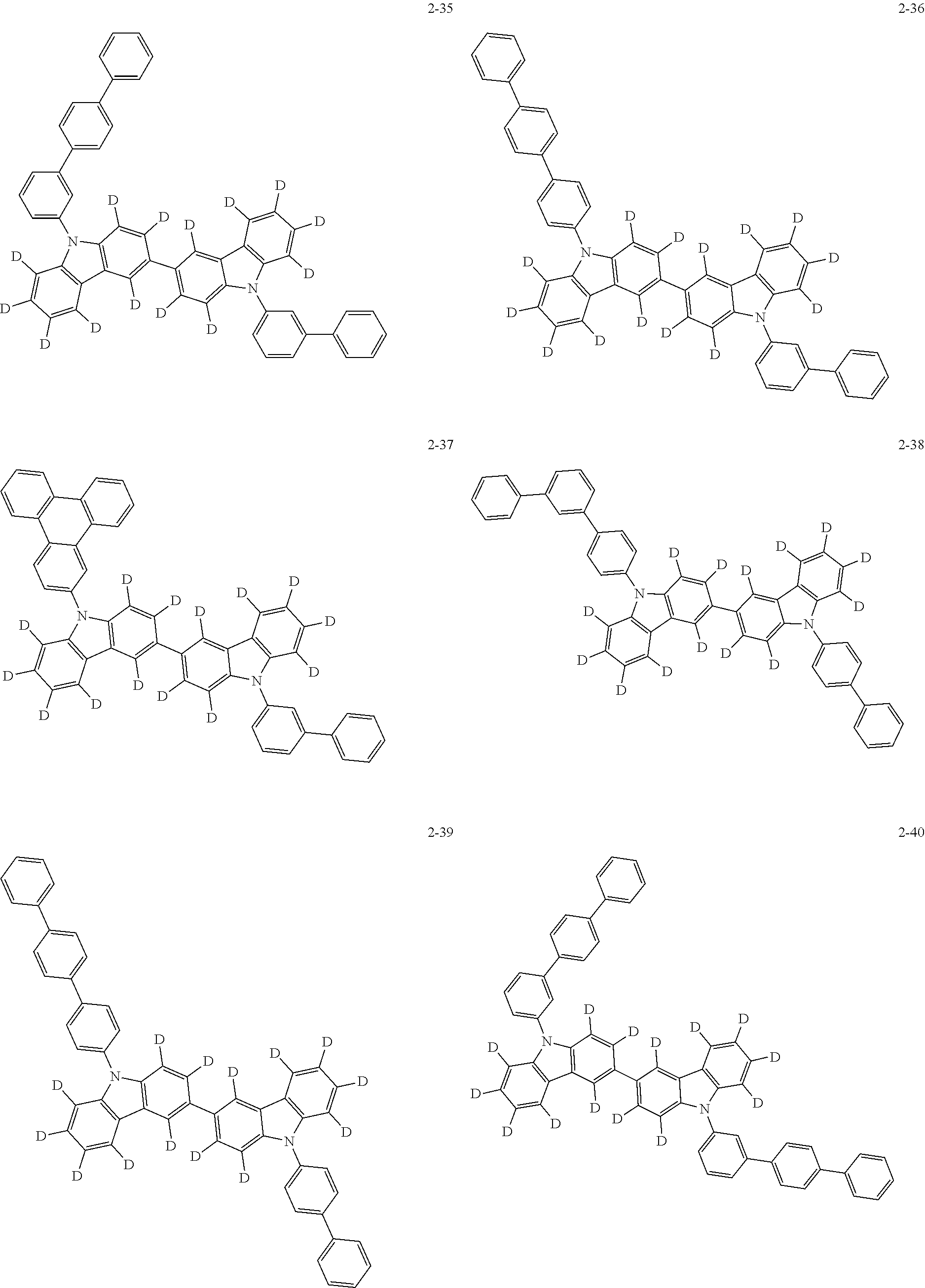

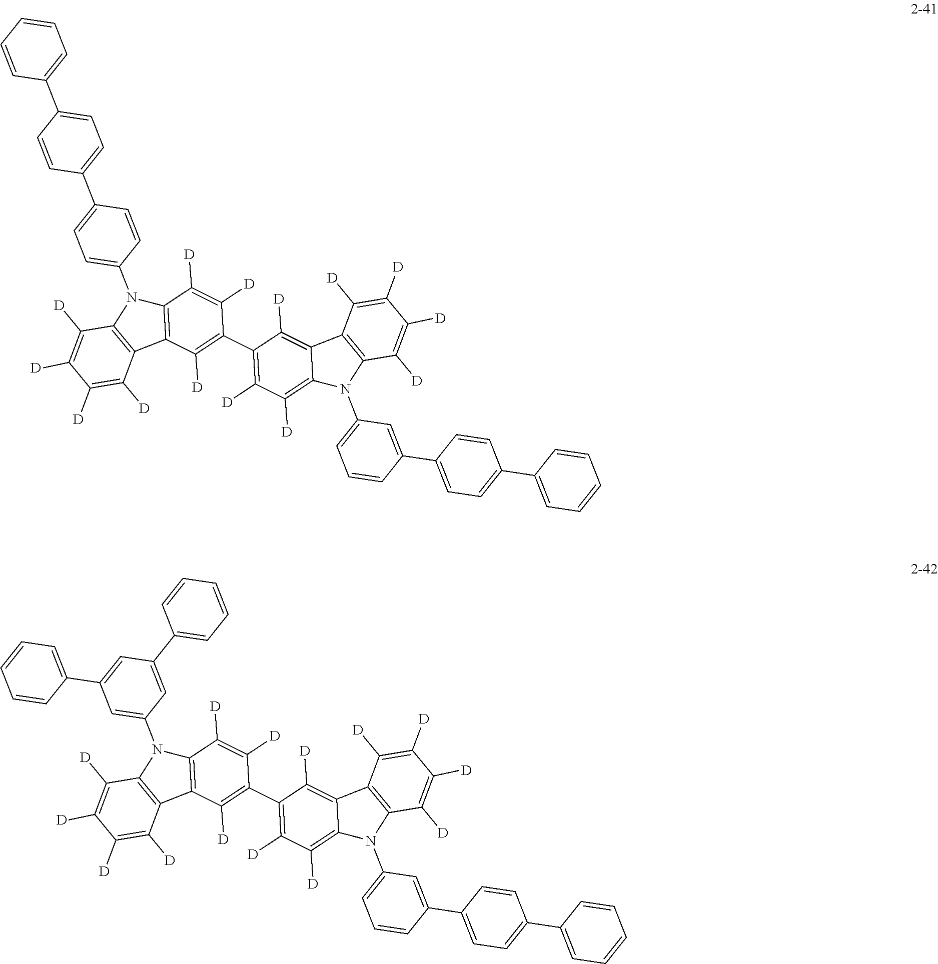 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1ccc(-c2ccc(-c3ccccc3)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1cccc(-c2ccc(-c3ccccc3)cc2)c1]

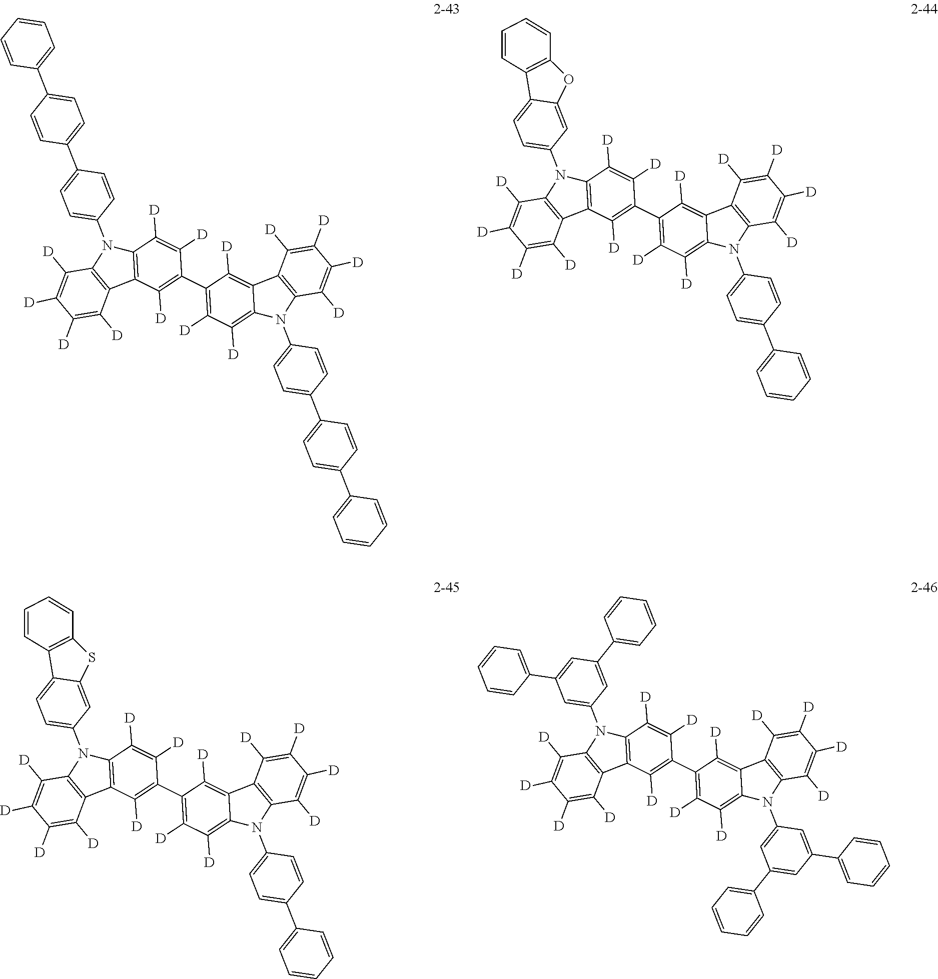

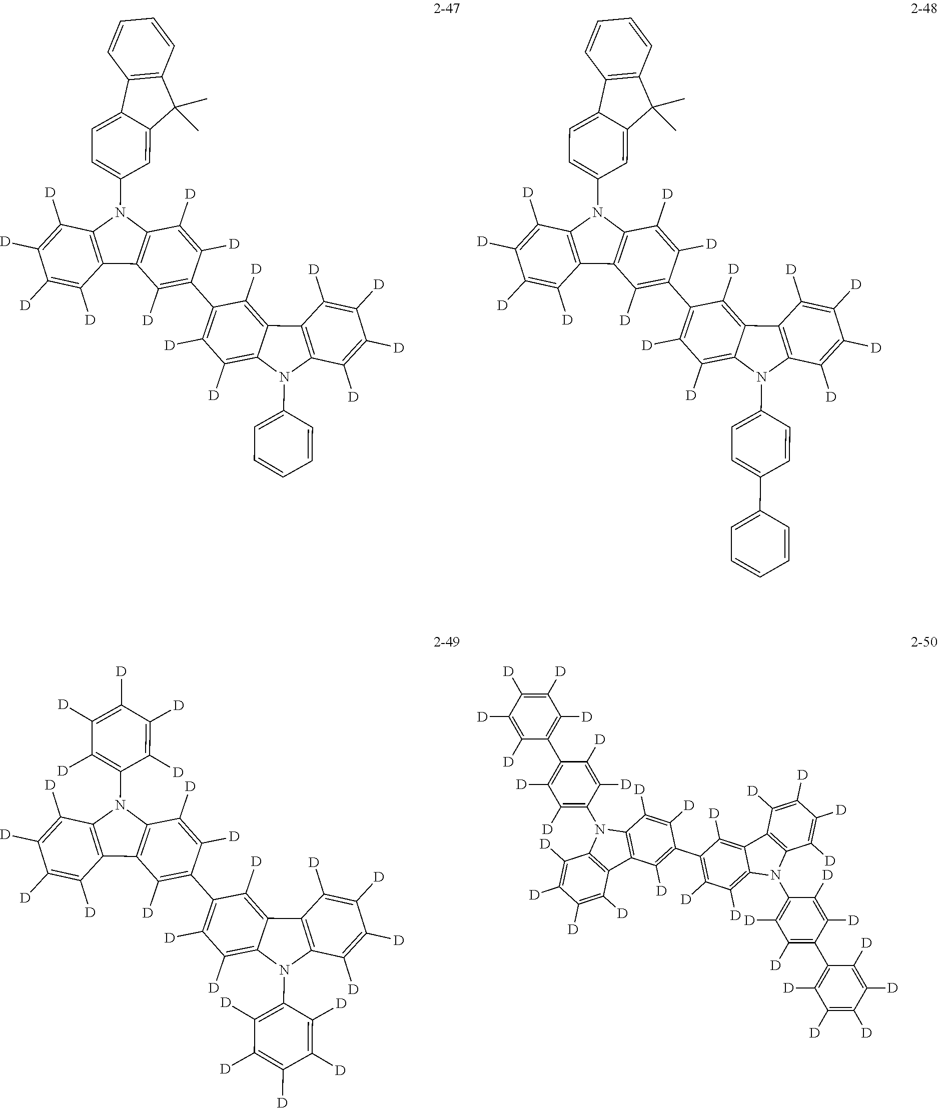

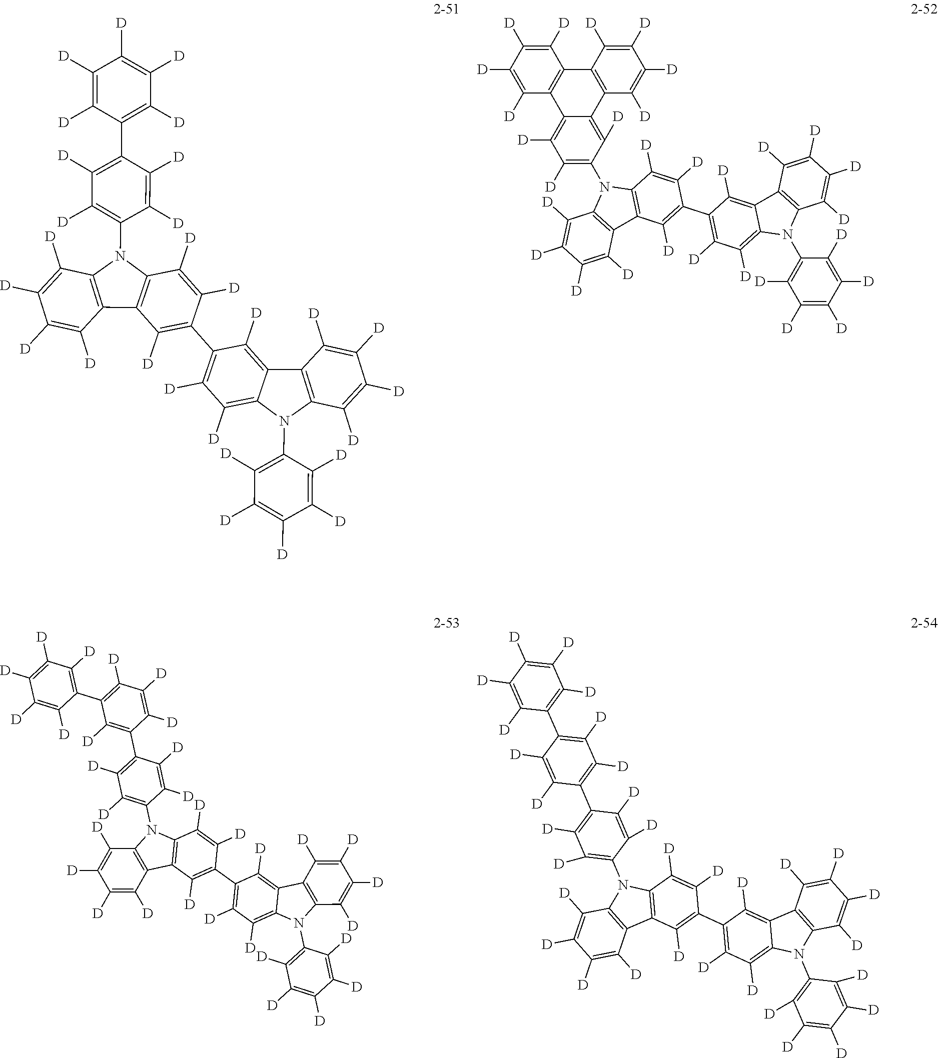

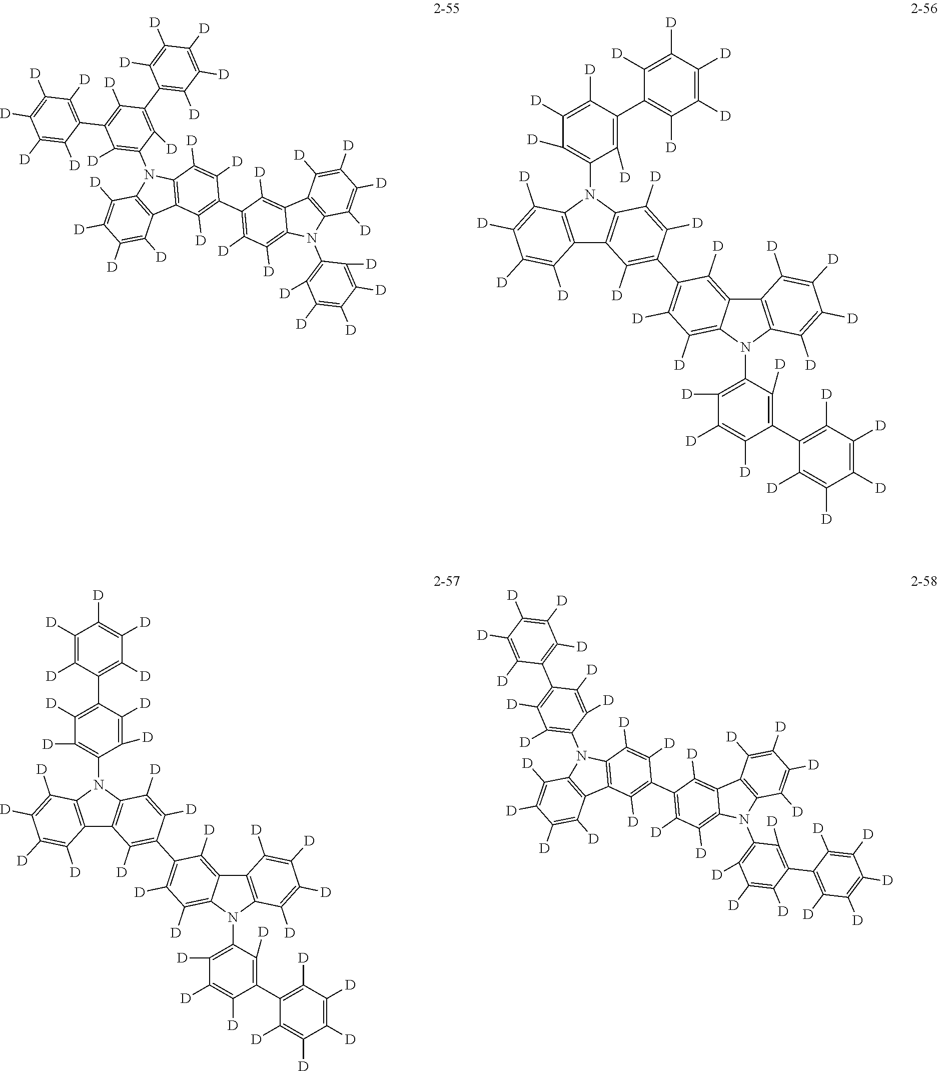

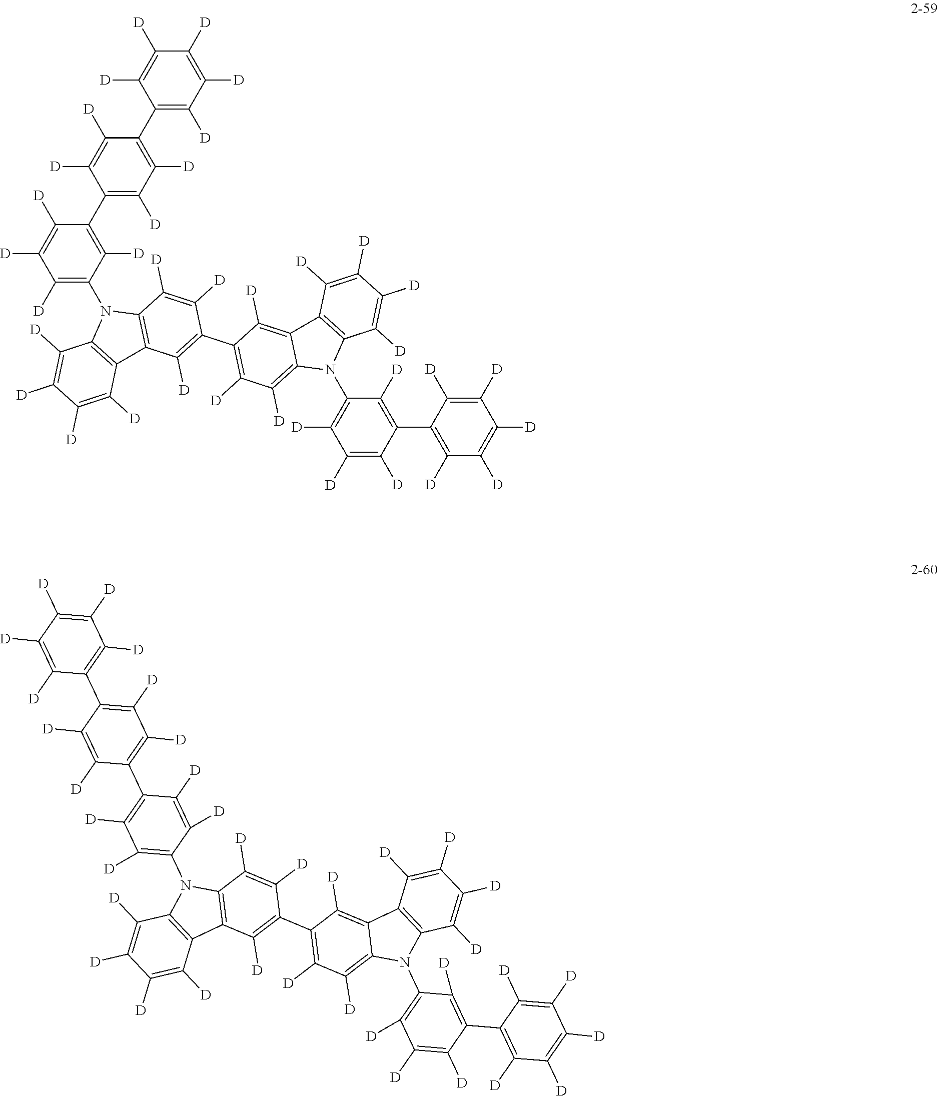

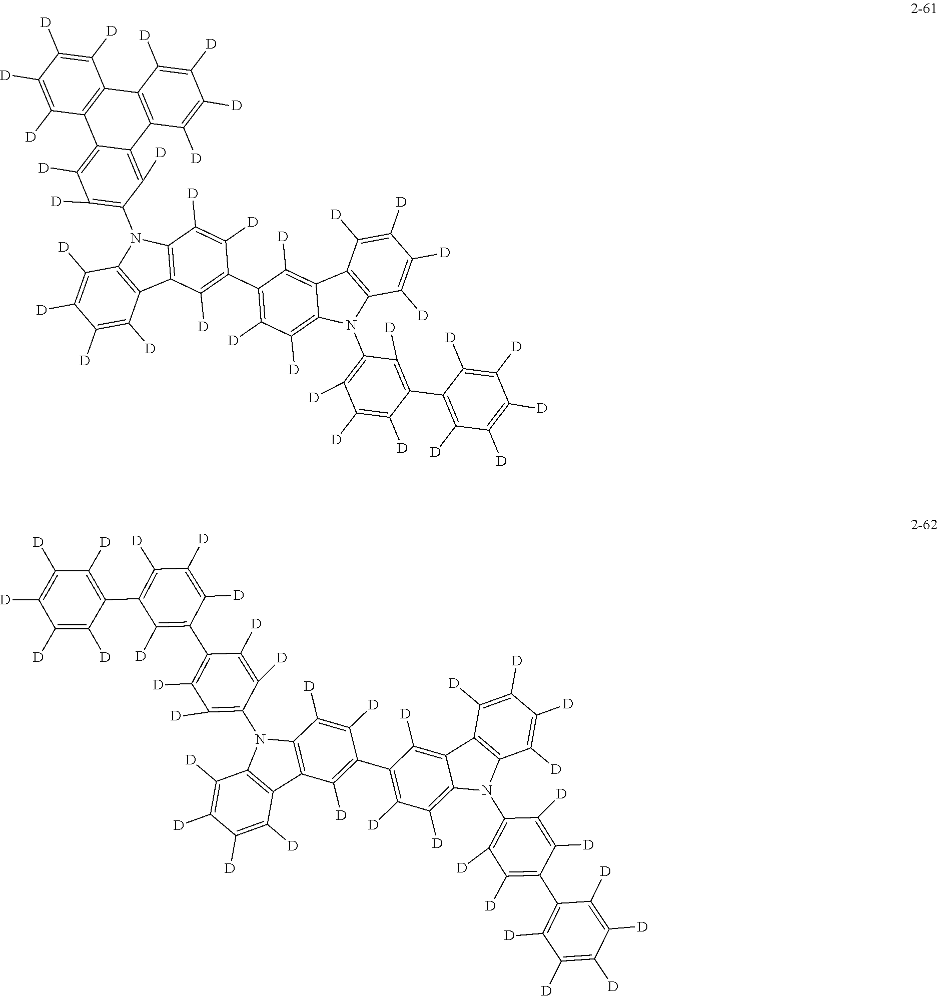

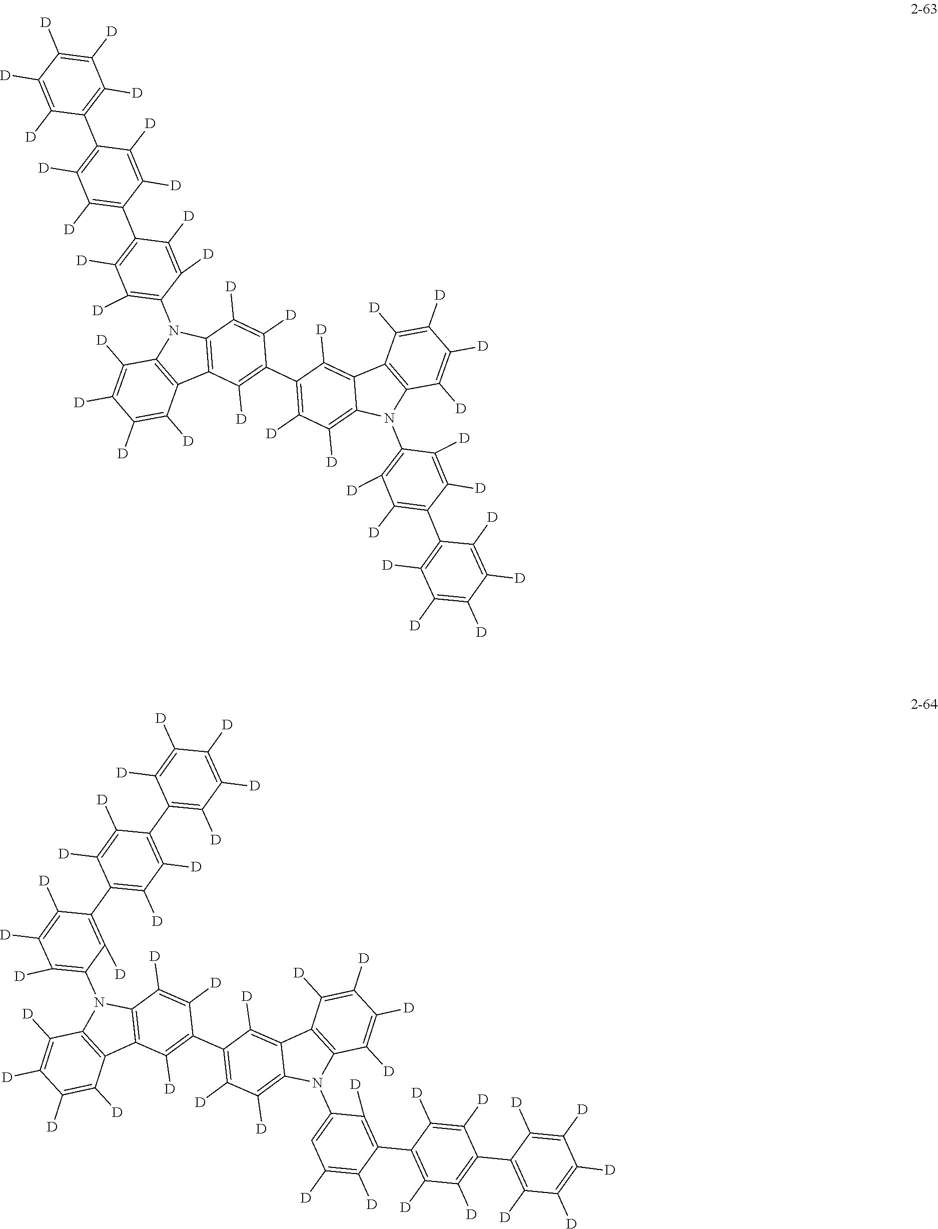

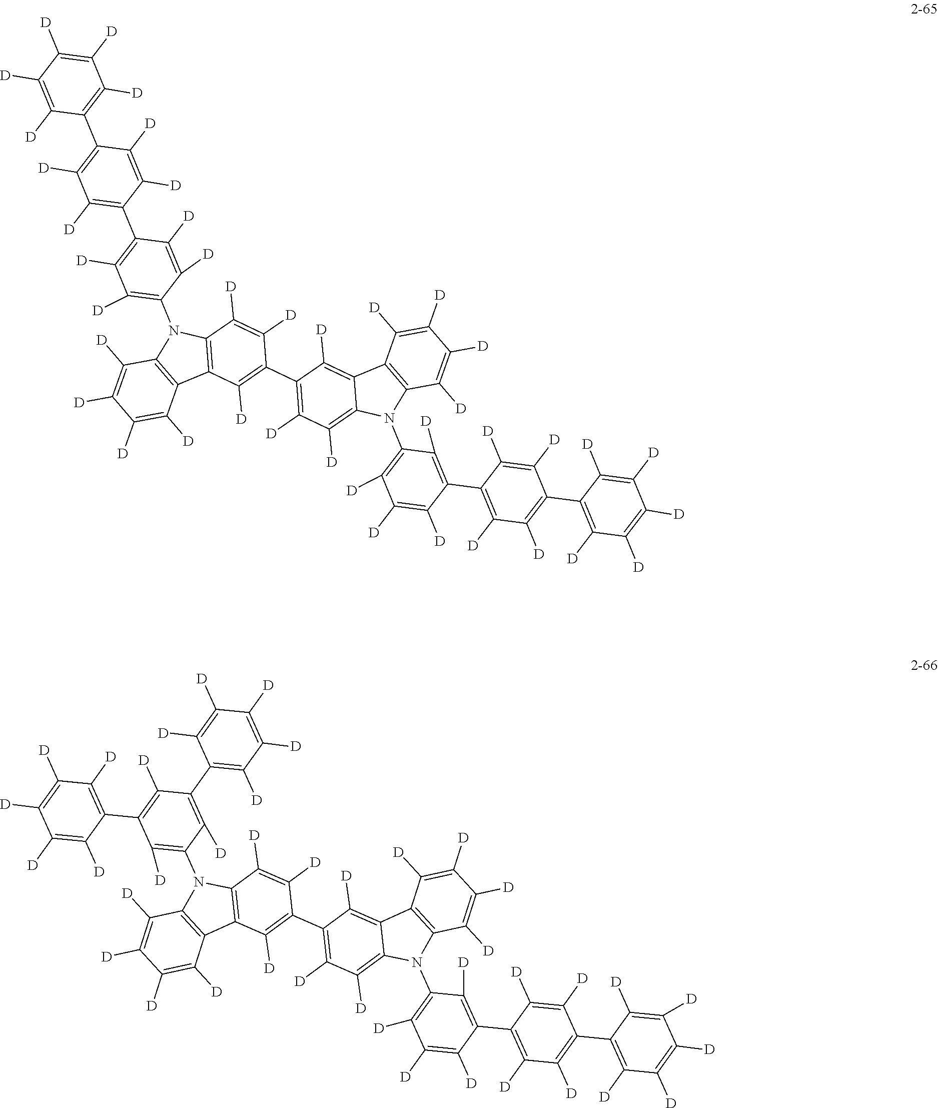

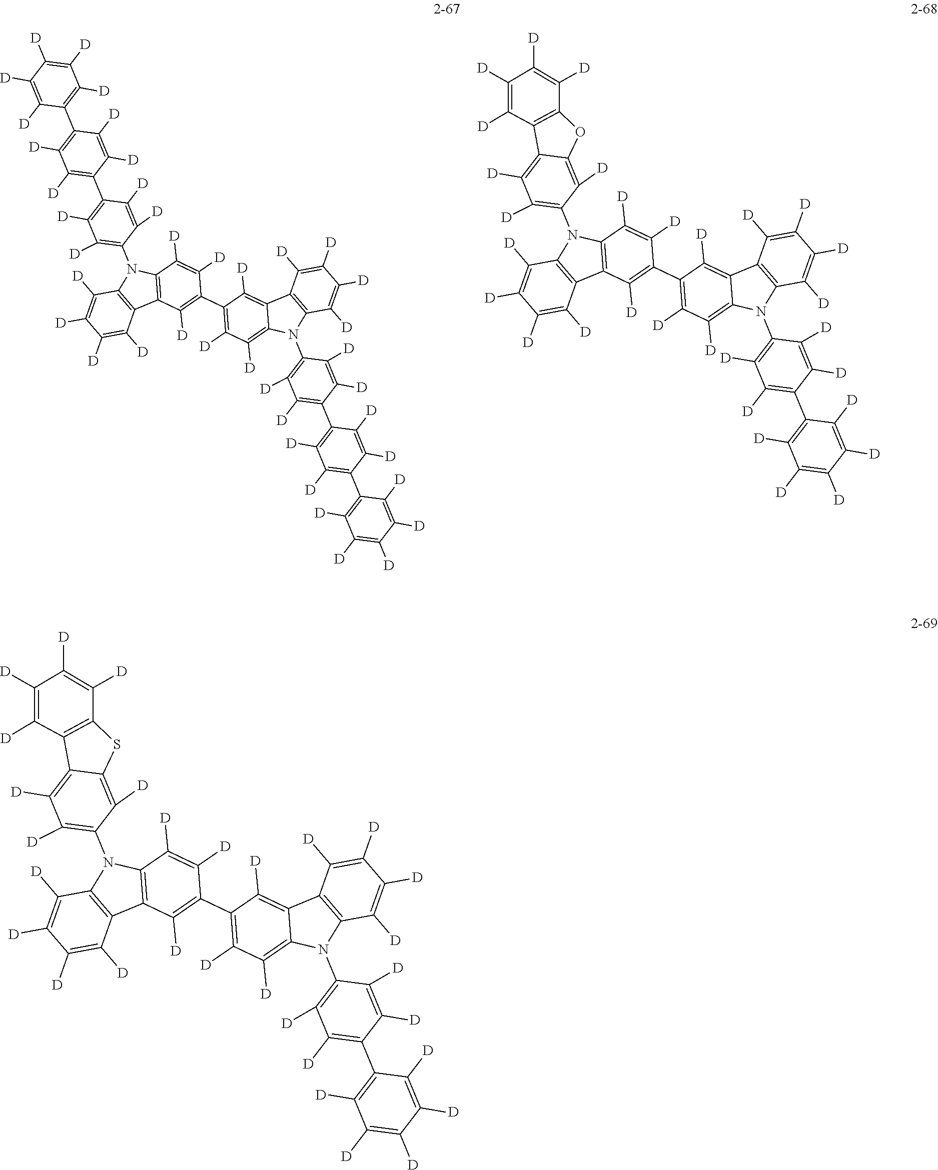

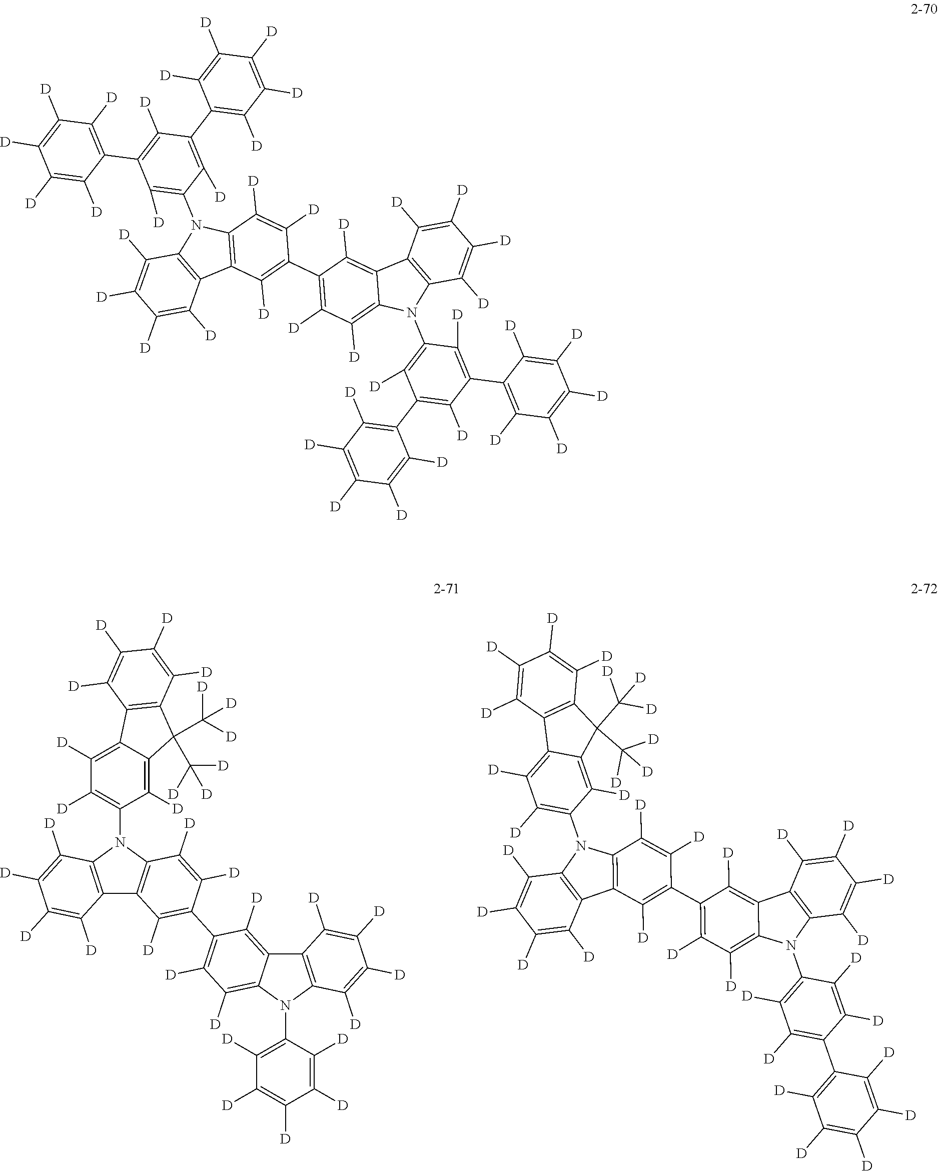

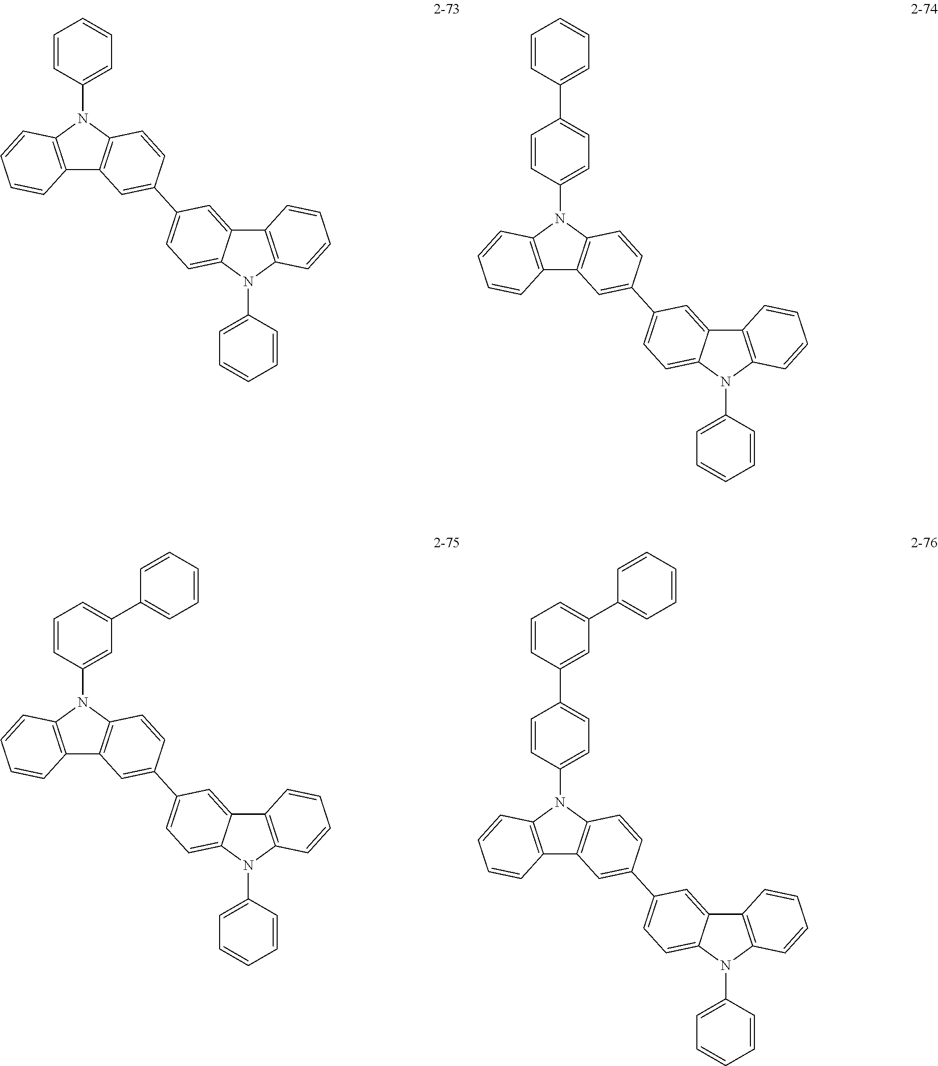 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

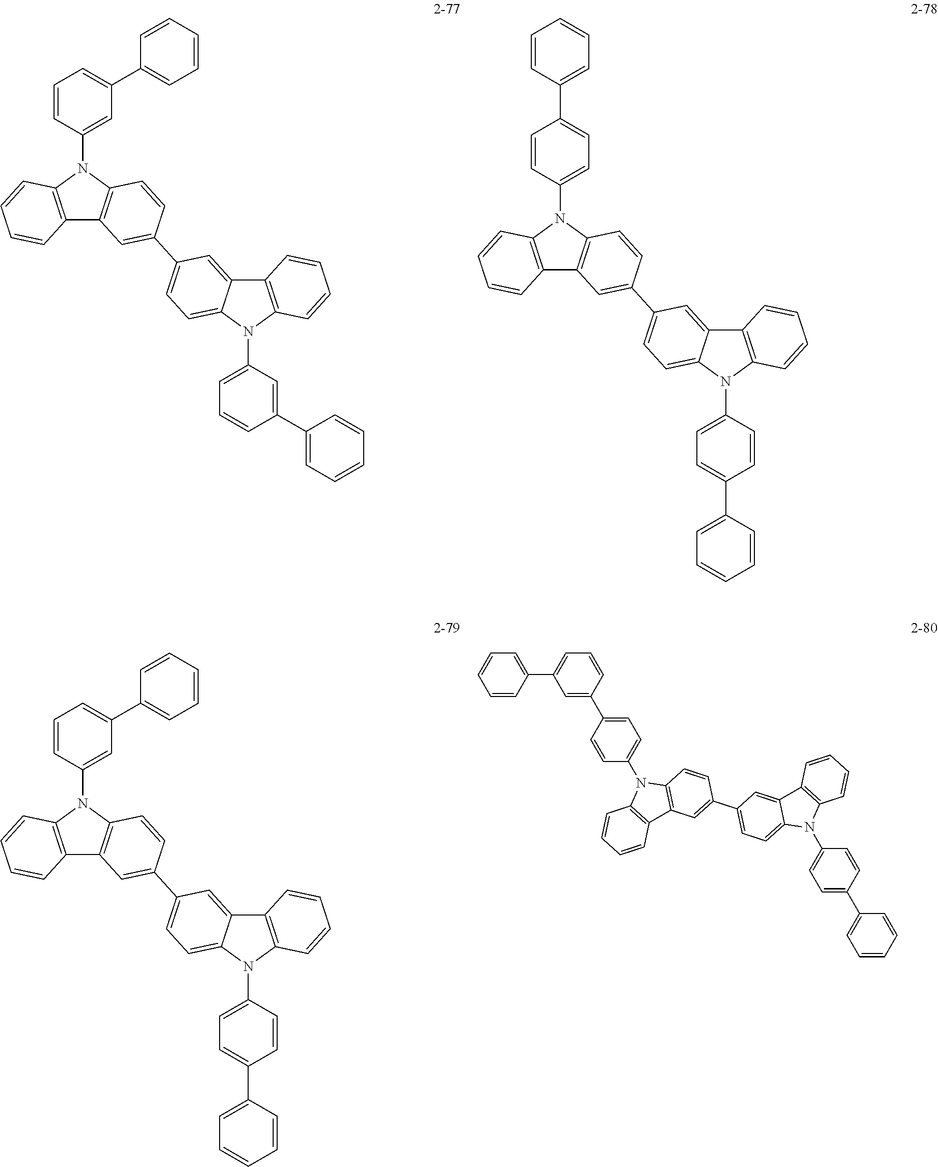 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1]

13. A composition for forming an organic material layer, comprising the heterocyclic compound of claim 1.

14. The composition of claim 13, further comprising a compound of the following Chemical Formula 2: 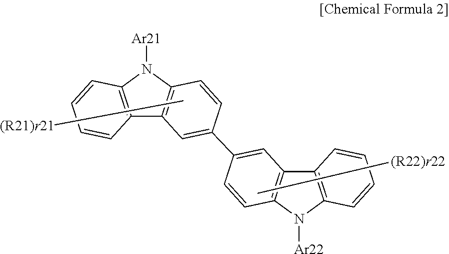
in Chemical Formula 2,
Ar21 and Ar22 are each independently a substituted or unsubstituted C6 to C60 aryl group; or a substituted or unsubstituted C2 to C60 heteroaryl group,
R21 and R22 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
r21 and r22 are each an integer from 0 to 7, and when r21 and r22 are each 2 or higher, substituents in the parenthesis are the same as or different from each other.

15. The composition of claim 14, wherein a weight ratio of the heterocyclic compound and the compound of Chemical Formula 2 is 1:10 to 10:1.
